(19) United States
(12) Patent Application Publication
(10) Pub. No.: US 2018/0118718 A1
(43) Pub. Date: May 3, 2018

(54) Substituted Quinoxaline Derivatives

(71) Applicant: Selvita S.A., Kraków (PL)

(72) Inventors: Charles-Henry Robert Yves Fabritius, Kraków (PL); Mateusz Oktawian Nowak, Kraków (PL); Katarzyna Anna Wiklik, Kraków (PL); Aleksandra Barbara Sabiniarz, Kraków (PL); Marcin Dominik Bien, Suloszowa (PL); Anna Malgorzata Buda, Kraków (PL); Pawel Szczepan Guzik, Kraków (PL); Arkadiusz Kacper Bialas, Wielun (PL); Henryk Edward Pawlik, Kraków (PL); Nicolas Felix Pierre Boutard, Kraków (PL)

(73) Assignee: Selvita S.A., Kraków (PL)

(21) Appl. No.: 15/573,541

(22) PCT Filed: May 12, 2016
(86) PCT No.: PCT/EP2016/000784

§ 371 Date: Nov. 13, 2017

(30) Foreign Application Priority Data
May 13, 2015 (EP) 15 001 444.7
May 13, 2015 (EP) 15 460 016.7

(51) Int. Cl. C07D 401/14; C07D 405/14; A61K 31/498; C07D 471/04; C07D 403/04; C07D 417/14; C07D 401/12; A61K 31/5377; C07D 403/14; A61K 31/506; C07D 498/04; A61K 31/5383; C07D 405/04; C07D 409/14; C07D 413/14; A61K 31/501; A61P 35/00; A61P 35/02

(52) U.S. Cl. CPC C07D 401/14; A61P 35/02; A61K 31/498; C07D 471/04; C07D 403/04; C07D 417/14; C07D 401/12; A61K 31/5377; C07D 403/14; A61K 31/506; C07D 498/04; A61K 31/5383; C07D 405/04; C07D 409/14; C07D 413/14; A61K 31/501; A61P 35/00; C07D 405/14

(57) ABSTRACT

The present invention relates to substituted quinoxaline derivatives. These compounds are useful for the prevention and/or treatment of several medical conditions including hyperproliferative disorders and diseases.

### FIELD OF THE INVENTION

The present invention relates to substituted quinoxaline derivatives. These compounds are useful for inhibiting 6-phosphofructo-2-kinase/fructose-2,6-bisphosphatase (PFKFB) and for the prevention and/or treatment of medical conditions affected by PFKFB activity.

### BACKGROUND OF THE INVENTION

Glycolysis is a non-oxidative metabolic pathway in which glucose is degraded by cells to generate ATP (adenosine triphosphate), i.e. energy. While normal, i.e. healthy cells are usually favoring this pathway for generating ATP—only under anaerobic conditions, many cancer cells generate ATP even in the presence of oxygen—from glucose via glycolysis; the glycolytic rate can be up to 200 times greater in malignant rapidly-growing tumor cells than in healthy cells. This switch of energy metabolism in cancer cells to the process of “aerobic glycolysis” is known as the “Warburg Effect” (D. G. Brooke et al., Biorganic & Medicinal Chemistry 22 (2014) 1029-1039; T. V. Pyrkov et al., ChemMedChem 2013, 8, 1322-1329).

The rate of glycolysis is regulated by several enzymes, including phosphofructokinase, that catalyze irreversible reactions in the course of glycolysis. 6-phosphofructo-1-kinase (PFK-1), the precursor of anaerobic ATP production, which converts fructose-6-phosphate (F6P) to fructose-1,6-bisphosphate (F1,6-BP), is considered to be the rate-limiting enzyme in the process of converting glucose into pyruvate. PFK-1 is allosterically activated by fructose-2,6-bisphosphate (F2,6-BP) which is synthesized from F6P by phosphofructokinase-2 (PFK-2; 6-phosphofructo-2-kinase/fructose-2,6-bisphosphatase, PFKFB). Four isoforms of the PFK-2 family are known, namely PFKFB1, PFKFB2, PFKFB3, and PFKFB4 (D. G. Brooke et al., Biorganic & Medicinal Chemistry 22 (2014) 1029-1039; T. V. Pyrkov et al., ChemMedChem 2013, 8, 1322-1329).

Many different cancer types exhibit an overexpression of PFK-2, particularly its isozymes PFKFB4 and hypoxia-inducible form PFKFB3. PFKFB3 is overexpressed in many cancer types including colon, prostate, pancreatic, breast, thyroid, leukemia, lung, ovarian tumors (D. G. Brooke et al., Biorganic & Medicinal Chemistry 22 (2014) 1029-1039; T. V. Pyrkov et al., ChemMedChem 2013, 8, 1322-1329). Overexpression of PFKFB4 has been associated, inter alia, with glioma, hepatic, bladder, and prostate cancer (T. V. Pyrkov et al., ChemMedChem 2013, 8, 1322-1329). Thus, 6-phosphofructo-2-kinase/fructose-2,6-bisphosphatase and in particular isoforms PFKFB3 and PFKFB4 are promising targets for cancer therapy by utilizing small molecules as inhibitors of these enzymes.

### DESCRIPTION OF THE INVENTION

It is an object of the present invention to provide inhibitors of PFKFB3 and/or PFKFB4 wherein that inhibitors may be useful for the prevention and/or treatment of medical conditions, disorders and/or diseases that are affected by PFKFB3 and/or PFKFB4 activity. It is a particular object of the present invention to provide such inhibitors for the treatment of hyperproliferative disorders, in particular cancer diseases.

The object has surprisingly been solved by compounds of formula (I)

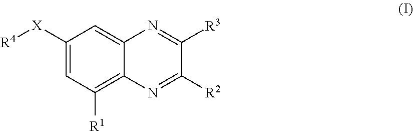

wherein
X denotes N—R⁵ or O;
R¹ denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, CA^{X};
R² and R³ denote independently from each other H, —OH, —SH, straight-chain or branched —C₁₋₆-alkyl, straight-chain or branched —C₂₋₆-alkenyl, straight-chain or branched —O—C₁₋₆-alkyl, straight-chain or branched —S—C₁₋₆-alkyl, HaI, —CN, —NH₂, —NH(C₁₋₄-alkyl), —N(C₁₋₄-alkyl)₂ which C₁₋₄-alkyl substituents may be the same or different and may be straight-chain or branched;
R⁴ denotes Ar^{W} or Hetar^{W}, which Ar^{W} or Hetar^{W} bears in its ortho-position (relative to the attachment of R⁴ to X) one (1) substituent R^{W1} and may or may not bear further substituents;
R⁵ denotes H, Ar^{X}, Hetar^{X}, Hetcyc^{X}, LA^{X}, CA^{X};
Ar^{W} denotes a mono-, bi- or tricyclic aromatic ring system with 5, 6, 7, 8, 9, 10, 11, 12, 13, 14 ring carbon atoms which ring system may bear—besides the ortho-substituent R^{W1}— no further substituent or one (1) further substituent R^{W2} or two (2) further substituents R^{W2}, R^{W3}, that may be the same or different;
Ar^{X} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{X1}, R^{X2}, R^{X3};
Ar^{Y} denotes a mono-, bi- or tricyclic aromatic ring system with 5, 6, 7, 8, 9, 10, 11, 12, 13, 14 ring carbon atoms which ring system may be unsubstituted or mono-, di- or trisubstituted with independently from each other R^{Y1}, R^{Y2}, R^{Y3};
Hetar^{W} denotes a mono-, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that ring system may bear—besides the ortho-substituent R^{W1}— no further substituent or one (1) further substituent R^{W2} or two (2) further substituents R^{W2}, R^{W3}, that may be the same or different;
Hetar^{X} denotes a mono-, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or tri-substituted with independently from each other R^{X1}, R^{X2}, R^{X3};
Hetar^{Y} denotes a mono-, bi- or tricyclic aromatic ring system with 5, 6, 7, 8, 9, 10, 11, 12, 13, 14 ring atoms wherein 1, 2, 3, 4, 5 of said ring atoms is/are a hetero atom(s) selected from N, O and/or S and the remaining are carbon atoms, wherein that aromatic ring system may be unsubstituted or mono-, di- or tri-substituted with independently from each other R^{Y1}, R^{Y2}, R^{Y3};
Hetcyc^{X} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{X4}; R^{X5}, R^{X6};
Hetcyc^{Y} denotes a saturated or partially unsaturated mono-, bi- or tricyclic heterocycle with 3, 4, 5, 6, 7, 8, 9, 10, 11, 12, 13, 14 ring atoms wherein 1, 2, 3, 4, 5 ring atom(s) is/are heteroatom(s) selected from N, O and/or S and the remaining ring atoms are carbon atoms, wherein that heterocycle may be unsubstituted or mono-, di- or trisubstituted with R^{Y4}; R^{Y5}; R^{Y6};
R^{W1} denotes HaI, LA^{X}, CA^{X}, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, —CN, —NO₂, —SO₂NH₂, —SO₂NHR^{W4}, —SO₂NR^{W4}R^{W6}, —NH—SO₂—R^{W6}, —NR^{W4}—SO₂—R^{W6}, —S—R^{W6}, —S(═O)—R^{W6}, —SO₂—R^{W6}, —NH₂, —NHR^{W4}, —NR^{W4}R^{W6}, —OH, —O—R^{W6}, —CHO, —C(═O)—R^{W6}, —COOH, —C(═O)—O—R^{W6}, —C(═O)—NH₂, —C(═O)—NHR^{W4}, —C(═O)—NR^{W4}R^{W6}, —NH—C(═O)—R^{W6}, —NR^{W4}—C(═O)—R^{W6}, —NH—(C₁₋₃-alkylene)-C(═O)—NH₂, —NH—(C₁₋₃-alkylene)-C(═O)—NHR^{W4}, —NH—(C₁₋₃-alkylene)-C(═O)—NR^{W4}R^{W6}, or
R^{W1} and R⁵ form together a divalent alkylene chain with 1, 2, 3, 4, 5 chain carbon atoms wherein 2 adjacent CH₂ groups may together be replaced by a —CH═CH— moiety, which divalent alkylene chain may be straight-chain or branched and may be unsubstituted or mono- or di-substituted with independently from each other straight-chain or branched —C₁₋₆-alkyl or ═O (oxo);
R^{W2}, R^{W3} denote independently from each other H, HaI, LA^{X}, CA^{X}, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, —CN, —NO₂, —SO₂NH₂, —SO₂NHR^{W4}, —SO₂NR^{W4}R^{W6}, —NH—SO₂—R^{W6}, —NR^{W4}—SO₂—R^{W6}, —S—R^{W6}, —S(═O)—R^{W6}, —SO₂—R^{W6}, —NH₂, —NHR^{W4}, —NR^{W4}R^{W6}, —NH—C(═O)—R^{W6}, —NR^{W4}—C(═O)—R^{W6}, —OH, —O—R^{W6}, —CHO, —C(═O)—R^{W6}, —COOH, —C(═O)—O—R^{W6}, —C(═O)—NH₂, —C(═O)—NHR^{W4}, —C(═O)—NR^{W4}R^{W6}, —C(═O)—NH—NH₂, —C(═O)—NH—NHR^{W4}, —NH—(C₁₋₃-alkylene)-C(═O)—NH₂, —NH—(C₁₋₃-alkylene)-C(═O)—NHR^{W4}, —NH—(C₁₋₃-alkylene)-C(═O)—NR^{W4}R^{W5},
   or
two of R^{W1}, R^{W2} and R^{W3} form a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), —O— —wherein that C₁₋₆-alkyl and C₁₋₄-alkyl radicals may be straight-chain or branched — and wherein 2 adjacent CH₂ groups may together be replaced by a —CH═CH— moiety, which divalent alkylene chain may be unsubstituted or mono- or di-substituted with independently from each other straight-chain or branched —C₁₋₆-alkyl or ═O (oxo);
R^{X1}, R^{X2}, R^{X3} denote independently from each other other H, HaI, LA^{X}, CA^{X}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}
   or
two of R^{X1}, R^{X2}, R^{X3} form a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), —O— —wherein that C₁₋₆-alkyl and C₁₋₄-alkyl radicals may be straight-chain or branched — and wherein 2 adjacent CH₂ groups may together be replaced by a —CH═CH— moiety, which divalent alkylene chain may be unsubstituted or mono- or di-substituted with independently from each other straight-chain or branched —C₁₋₆-alkyl or ═O (oxo);

R^{X4}, R^{X5}, R^{X6} denote independently from each other H, HaI, LA^{X}, CA^{X}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O);
R^{Y1}, R^{Y2}, R^{Y3} denote independently from each other H, HaI, LA^{Y}, CA^{Y}, —CN, —NO₂, —SF₅, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, —S(═O)—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}
   or
two of R^{Y1}, R^{Y2}, R^{Y3} form a divalent alkylene chain with 3, 4, 5 chain carbon atoms wherein 1 or 2 non-adjacent CH₂ groups of the divalent alkylene chain may be replaced independently from each other by —N(H)—, —N(—C(═O)—C₁₋₄-alkyl), —O— —wherein that C₁₋₆-alkyl and C₁₋₄-alkyl radicals may be straight-chain or branched — and wherein 2 adjacent CH₂ groups may together be replaced by a —CH═CH— moiety, which divalent alkylene chain may be unsubstituted or mono- or di-substituted with independently from each other straight-chain or branched
   C₁₋₆-alkyl or ═O (oxo);
R^{Y4}, R^{Y5}, R^{Y6} denote independently from each other H, HaI, LA^{Y}, CA^{Y}, —CN, —NO₂, —SF₅, —SO₂NH₂, —SO₂NHR^{Y7}, —SO₂NR^{Y7}R^{Y8}, —NH—SO₂—R^{Y9}, —NR^{Y7}—SO₂—R^{Y9}, —S(═O)—R^{Y9}, —SO₂—R^{Y9}, —NH₂, —NHR^{Y7}, —NR^{Y7}R^{Y8}, OH, O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, oxo (═O);
LA^{X} denotes straight-chain or branched C₁₋₆-alkyl which may be unsubstituted or mono-, di- or trisubstituted with independently from each other HaI, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X1}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O), wherein 1 or 2 non-adjacent CH₂ groups of the C₁₋₆-alkyl radical may independently from each other be replaced by O, S, N(H) or N—R^{X7} and/or 1 or 2 non-adjacent CH groups of the C₁₋₆-alkyl radical may independently from each other be replaced by N;
LA^{Y} denotes straight-chain or branched C₁₋₆-alkyl which may be unsubstituted or mono-, di- or trisubstituted with independently from each other HaI, —CN, —NO₂, —SF₅, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, —S(═O)—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, oxo (═O), wherein 1 or 2 non-adjacent CH₂ groups of the C₁₋₆-alkyl radical may independently from each other be replaced by O, S, N(H) or N—R^{Y7} and/or 1 or 2 non-adjacent CH groups of the C₁₋₆-alkyl radical may independently from each other be replaced by N;
LA^{Z} denotes a divalent straight-chain or branched C₁₋₆-alkylene radical which alkylene radical may be unsubstituted or mono-, di- or trisubstituted with independently from each other HaI, —CN, —NO₂, —SF₅, —SO₂NH₂, —SO₂NHR^{Z7}, —SO₂NR^{Z7}R^{Z5}, —NH—SO₂—R^{Z9}, —NR^{Z7}—SO₂—R^{Z9}, —S—R^{Z9}, —S(═O)—R^{Z9}, —SO₂—R^{Z9}, —NH₂, —NHR^{Z7}, —NR^{Z7}R^{Z8}, —OH, —O—R^{Z9}, —CHO, —C(═O)—R^{Z9}, —COOH, —C(═O)—O—R^{Z9}, —C(═O)—NH₂, —C(═O)—NHR^{Z7}, —C(═O)—NR^{Z7}R^{Z8}, —NH—C(O)—R^{Z9}, —NR^{Z7}—C(═O)—R^{Z9}, —NH—(C₁₋₃-alkylene)-C(═O)—NH₂, —NH—(C₁₋₃-alkylene)-C(═O)—NHR^{Z7}, —NH—(C₁₋₃-alkylene)-C(═O)—NR^{Z7}R^{Z8}, oxo (═O), wherein 1 or 2 non-adjacent CH₂ groups of that divalent alkylene radical may be replaced independently from each other by O, S, —N(H) or N—R^{Z7} and/or 1 or 2 non-adjacent CH groups of that divalent alkylene radical may be replaced by N;
R^{W4}, R^{W5}, R^{W6} denote Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, CA^{X}
   or
R^{W4} and R^{W5} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X7}, R^{X5}, R^{X9}, R^{Y7}, R^{Y8}, R^{Y9}, R^{Z7}, R^{Z8}, R^{Z9} denote independently from each other straight-chain or branched C₁₋₆-alkyl, which may be unsubstituted, which is preferred, or mono-, di- or trisubstituted with independently from each other HaI, —CN, —NO₂, —SF₅, —SO₂NH₂, —SO₂NHR^{X7v}, —SO₂NR^{X7v}R^{X8v}, —NH—SO₂— R^{X9v}, —NR^{X7v}—SO₂R^{X9v}, —S—R^{X9v}, —S(═O)—R^{X9v}, —SO₂—R^{X9v}, —NH₂, —NHR^{X7v}, —NR^{X7v}R^{X8v}, —OH, —O—R^{X9v}, —CHO, —C(═O)—R^{X9v}, —COOH, —C(═O)—O—R^{X9v}, —C(═O)—NH₂, —C(═O)—NHR^{X7v}, —C(═O)—NR^{X7v}R^{X8v}, —NH—C(═O)—R^{X9v}, —NR^{X7v}—C(═O)—R^{X9v}, —NH—(C₁₋₃-alkylene)-C(═O)—NH₂, —NH—(C₁₋₃-alkylene)-C(═O)—NHR^{X7v}, —NH—(C₁₋₃-alkylene)-C(═O)—NR^{X7v}R^{X8v}, oxo (═O), wherein 1 or 2 non-adjacent CH₂ groups of the C₁₋₆-alkyl radical may independently from each other be replaced by O, S, N(H) or N—R^{X7v} and/or 1 or 2 non-adjacent CH groups of the C₁₋₆-alkyl radical may independently from each other be replaced by N, or a saturated monocyclic carbocycle with 3, 4, 5, 6, 7 carbon atoms, which may be unsubstituted, which is preferred, or mono- or disubstituted with independently from each other HaI, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, —CN, —NO₂, —SF₅, —SO₂NH₂, —SO₂NHR^{X7v}, —SO₂NR^{X7v}R^{X8v}, —NH—SO₂—R^{X9v}, —NR^{X7v}—SO₂—R^{X9v}, —S—R^{X9v}, —S(═O)—R^{X9v}, —SO₂—R^{X9v}, —NH₂, —NHR^{X7v}, —NR^{X7v}R^{X8v}, —OH, —O—R^{X9v}, —CHO, —C(═O)—R^{X9v}, —COOH, —C(═O)—O—R^{X9v}, —C(═O)—NH₂, —C(═O)—NHR^{X7v}, —C(═O)—NR^{X7v}R^{X8v}, —NH—C(═O)—R^{X9v}, —NR^{X7v}—C(═O)—R^{X9v}, —NH—(C₁₋₃-alkylene)-C(═O)—NH₂, —NH—(C₁₋₃-alkylene)-C(═O)—NHR^{X7v}, —NH—(C₁₋₃-alkylene)-C(═O)—NR^{X7v}R^{X8v}, oxo (═O), with the proviso that if any of the substituents of that monocyclic carbocycle is Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, then any radical R^{X7}, R^{X8}, R^{X9}, R^{Y7}, R^{Y8}, R^{Y9}, R^{Z7}, R^{Z8}, R^{Z9} of any substituent of Ar^{X}, Ar^{Y}, Hetar^{X}, Hetar^{Y}, Hetcyc^{X}, Hetcyc^{Y}, LA^{X} and LA^{Z} may not denote a mono- or disubstituted monocyclic carbocycle, or a saturated monocyclic heterocycle with 3, 4, 5, 6, 7 ring atoms wherein 1 or 2 ring atom(s) is/are heteroatom(s) selected from N, O and/or S and the remaining ring atoms are carbon atoms, wherein that heterocycle may be unsubstituted or substituted with straight-chain or branched C₁₋₆-alkyl, —C(═O)—C₁₋₆-alkyl (straight-chain or branched) and/or oxo (═O), or a phenyl, —CH₂-phenyl, -naphthyl, —CH₂-naphthyl, heteroaromatic ring system or CH₂-heteroaromatic ring system with 5, 6, 7, 8, 9, 10, 11 ring atoms, wherein 1, 2, 3, 4, 5 of said ring atoms of said heteroaromic ring system is/are hetero atom(s) selected from N, O and/or S and the remaining are carbon atoms, wherein said phenyl, naphthyl or heteroaromatic ring system may be unsubstituted or mono-, di- or trisubstituted with independently from each other straight-chain or branched C₁₋₆-alkyl or O—C₁₋₆-alkyl, HaI or C(═O)—C₁₋₆-alkyl (straight-chain or branched);
   or
each pair R^{X7} and R^{X8}; R^{Y7} and R^{Y8}; R^{Z7} and R^{Z8} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X7v}, R^{X8v}, R^{X9v} denotes independently from each other straight-chain or branched C₁₋₆-alkyl, which may be unsubstituted or mono-, di- or trisubstituted with HaI, or a unsubstituted saturated monocyclic carbocycle with 3, 4, 5, 6, 7 carbon atoms;
   or
R^{X7v} and R^{X8v} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
CA^{X}, CA^{Y} denote independently from each other a saturated monocyclic carbocycle with 3, 4, 5, 6, 7 carbon atoms which carbocycle may be unsubstituted or mono- or disubstituted with independently from each other R^{CA1}, R^{CA2};
R^{CA1}, R^{CA2} denote independently from each other H, HaI, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X8}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, oxo (═O), with the proviso that if R^{CA1} or R^{CA2} denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{Z}-Ar^{Y}, LA^{Z}-Hetar^{Y}, LA²-Hetcyc^{Y}, then Ar^{X}, Ar^{Y}, Hetar^{X}, Hetar^{Y}, Hetcyc^{X}, Hetcyc^{Y} may not be substituted with CA^{X} or CA^{Y};
   HaI denotes F, Cl, Br, I;
   or derivatives, N-oxides, prodrugs, solvates, tautomers or stereoisomers thereof as well as the physiologically acceptable salts of each of the foregoing, including mixtures thereof in all ratios.

In general, all residues which occur more than once may be identical or different, i.e. are independent of one another. Above and below, the residues and parameters have the meanings indicated for formula (I), unless expressly indicated otherwise. Accordingly, the invention relates, in particular, to the compounds of formula (I) in which at least one of the said residues has one of the preferred meanings indicated below.

Any of those preferred or particular embodiments of the present invention as specified below and in the claims do not only refer to the specified compounds of formula (I) but to derivatives, N-oxides, prodrugs, solvates, tautomers or stereoisomers thereof as well as the physiologically acceptable salts of each of the foregoing, including mixtures thereof in all ratios, too, unless indicated otherwise.

In a particular embodiment, PE1, the compounds of the present invention are compounds of formula (I)

wherein
X denotes N—R⁵ or O;
R¹ denotes Ar^{X}, Hetar^{X}, Ar^{X}—Ar^{Y}, Ar^{X}-Hetar^{Y};
R² and R³ both denote H;
R⁴ denotes Ar^{W} or Hetar^{W}, which Ar^{W} or Hetar^{W} has in its ortho-position (relative to the attachment of R⁴ to X) one (1) substituent R^{W1} and may or may not bear further substituents;
R⁵ denotes H or LA^{X}, in particular H or straight-chain or branched C₁₋₆-alkyl, preferably H;
Ar^{W} denotes a monocyclic aromatic ring system with 6 ring carbon atoms which ring system may bear—besides the ortho-substituent R^{W1} —no further substituent or one (1) further substituent R^{W2}, wherein R^{W1} and R^{W2} may be the same or different;
Ar^{X} denotes a monocyclic aromatic ring system with 6 ring carbon atoms which ring system may be unsubstituted or mono- or di-substituted with independently from each other R^{X1}, R^{X2};
Ar^{Y} denotes a monocyclic aromatic ring system with 6 ring carbon atoms which ring system may be unsubstituted or mono- or di-substituted with independently from each other R^{Y1}, R^{Y2};
Hetar^{W} denotes a monocyclic aromatic ring system with 5 or 6 ring atoms wherein 1, 2 or 3 of said ring atoms is/are nitrogen atom(s) and the remaining are carbon atoms, wherein that ring system may bear besides the ortho-substituent R^{W1} no further substituent or one (1) further substituent R^{W2} wherein R^{W1} and R^{W2} may be the same or different;
Hetar^{X} denotes a mono- or bi-cyclic aromatic ring system with 5, 6, 9, 10 ring atoms wherein 1, 2, 3 or 4 of said ring atoms is/are a hetero atom(s) selected from N, O and/or S and the remaining are carbon atoms, wherein that aromatic ring system may be unsubstituted or mono- or di-substituted with independently from each other R^{X1}, R^{X2};
Hetar^{Y} denotes a monocyclic aromatic ring system with 5 or 6 ring atoms wherein 1, 2 or 3 of said ring atoms is/are a nitrogen atom(s) and the remaining are carbon atoms, wherein that aromatic ring system may be unsubstituted or mono-substituted with R^{Y1};
Hetcyc^{X} denotes a saturated mono-cyclic heterocycle with 4, 5, 6, 7, ring atoms wherein 1 or 2 ring atom(s) is/are heteroatom(s) selected from N, O and/or S and the remaining ring atoms are carbon atoms, wherein that heterocycle may be unsubstituted or mono-, di- or trisubstituted with R^{X4}, R^{X5}, R^{X6};
Hetcyc^{Y} denotes a saturated monocyclic heterocycle with 4, 5, 6, 7 ring atoms wherein 1 or 2 ring atom(s) is/are heteroatom(s) selected from N, O and/or S and the remaining ring atoms are carbon atoms, wherein that heterocycle may be unsubstituted or mono-, di- or trisubstituted with R^{Y4}, R^{Y5}, R^{Y6};
R^{W1} denotes LA^{X}, Hetar^{X}, Hetcyc^{X}, HaI, —CN, —OH, —O—R^{W6}, —SO₂NH₂, —SO₂NHR^{W4}, —SO₂NR^{W4}R^{W5}, —NH—SO₂—R^{W6}, —NR^{W4}—SO₂—R^{W6}, —SO₂—R^{W6}, —NH₂, —NHR^{W4}, —NR^{W4}R^{W5}, —C(═O)—OH, —C(═O)—O—R^{W6}, —C(═O)—NH₂, —C(═O)—NHR^{W4}, —C(═O)—NR^{W4}R^{W5}, —NH—C(═O)—R^{W6}, —NR^{W4}—C(═O)—R^{W6};
or R⁵ and R^{W1} form together a divalent alkylene chain with 1, 2, 3 chain carbon atoms;
R^{W2} denotes H, Hetar^{X}, Hetcyc^{X}, HaI, LA^{X}, —CN, —OH, —O—R^{W6}, —NO₂, —NH₂, —NHR^{W4}, —NR^{W4}R^{W5}, —C(═O)—OH, —C(═O)—O—R^{W6}, —C(═O)—NH₂, —C(═O)—NHR^{W4}, —C(═O)—NR^{W4}R^{W5}, —C(═O)—NH—NH₂, —NH—C(═O)—R^{W6}, —NR^{W4}—C(═O)—R^{W6};
or R^{W1} and R^{W2} form together a divalent alkylene chain with 3, 4, 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), —O— —wherein that C₁₋₆-alkyl and C₁₋₄-alkyl radicals may be straight-chain or branched — and wherein 2 adjacent CH₂ groups may together be replaced by a —CH═CH— moiety, which divalent alkylene chain may be unsubstituted or mono- or di-substituted with independently from each other straight-chain or branched —C₁₋₆-alkyl or ═O (oxo);
R^{X1}, R^{X2} denote independently from each other H, LA^{X}, —NH₂, —NHR^{X7}, —NR^{X7}R^{X5}; HaI, —OH, —OR^{X9}, —SR^{X9}, —SF₅, —C(═O)—NH₂, —C(═O)—NHR^{X7}, —C(═O)—NR^{X7}R^{X8}, —NH—C(═O)—R^{X9},
   or form a divalent alkylene chain with 3, 4, 5 chain carbon atoms wherein 1 or 2 non-adjacent CH₂ group(s) of the divalent alkylene chain may be replaced independently from each other by —O—, which divalent alkylene chain may be unsubstituted or mono- or di-substituted with independently from each other straight-chain or branched —C₁₋₆-alkyl;
   R^{Y1}, R^{Y2} denote independently from each other LA^{Y};
   LA^{X} denotes straight-chain or branched C₁₋₆-alkyl which may be unsubstituted or mono-, di- or trisubstituted with independently from each other HaI, —CN, —NH₂, —NHR^{X7}, —NR^{X7}R^{X8};
   LA^{Y} denotes straight-chain or branched C₁₋₆-alkyl;
   LA^{Z} denotes a divalent straight-chain or branched C₁₋₆-alkylene radical;
   R^{X4}, R^{X5}, R^{X6} denote independently from each other H, HaI, LA^{X}, —C(═O)—R^{X9}, oxo (═O);

R^{Y4}, R^{Y5}, R^{Y6} denote independently from each other H, HaI, LA^{Y}, —C(═O)—R^{Y9}, oxo (═O);
R^{W4} denotes straight-chain or branched C₁₋₆-alkyl, saturated monocyclic carbocycle with 3, 4, 5, 6, 7 carbon atoms, Ar^{X}, Hetar^{X}, Hetcyc^{X}, LA^{Z}-Ar^{Y}, LA^{Z}-Hetar^{Y} or LA^{Z}-Hetcyc^{Y};
R^{W5}, R^{W6} denote independently from each other straight-chain or branched C₁₋₆-alkyl or a saturated monocyclic carbocycle with 3, 4, 5, 6, 7 carbon atoms, Ar^{X}, Hetar^{X}, Hetcyc^{X}, LA^{Z}-Ar^{Y}, LA^{Z}-Hetar^{Y} or LA^{Z}-Hetcyc^{Y}
or
R^{W4} and R^{W5} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X7}, R^{X8}, R^{X9}, R^{Y9} denote independently from each other straight-chain or branched C₁₋₆-alkyl, which may be unsubstituted or mono-, di- or trisubstituted with HaI or monosubstituted with NH₂, a saturated monocyclic carbocycle with 3, 4, 5, 6, 7 carbon atoms, or a saturated monocyclic heterocycle with 3, 4, 5, 6, 7 ring atoms wherein 1 or 2 ring atom(s) is/are heteroatom(s) selected from N, O and/or S and the remaining ring atoms are carbon atoms, wherein that heterocycle may be unsubstituted or substituted with straight-chain or branched C₁₋₆-alkyl, —C(═O)—C₁₋₆-alkyl (straight-chain or branched) and/or oxo (═O), or a phenyl, —CH₂-phenyl, -naphthyl, —CH₂-naphthyl, heteroaromatic ring system or CH₂-heteroaromatic ring system with 5, 6, 7, 8, 9, 10, 11 ring atoms, wherein 1, 2, 3, 4, 5 of said ring atoms of said heteroaromic ring system is/are hetero atom(s) selected from N, O and/or S and the remaining are carbon atoms, wherein said phenyl, naphthyl or heteroaromatic ring system may be unsubstituted or mono-, di- or trisubstituted with independently from each other straight-chain or branched C₁₋₆-alkyl or O—C₁₋₆-alkyl, HaI or C(═O)—C₁₋₆-alkyl (straight-chain or branched)
or
R^{X7} and R^{X8} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
HaI denotes F, Cl, Br, I;
or derivatives, N-oxides, prodrugs, solvates, tautomers or stereoisomers thereof as well as the physiologically acceptable salts of each of the foregoing, including mixtures thereof in all ratios.

In another particular embodiment, PE1a, of the present invention which may also be an embodiment of particular embodiment PE1 the substituent R¹, that denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, CA^{X}, is attached to the core quinoxaline ring system of formula (I) via a carbon atom.

A further particular embodiment of the present invention, PE2, which may optionally be part of the above described particular embodiments PE1 and/or PE1a comprises compounds of formula (I) wherein
X denotes N—R⁵ or 0, in particular N—R⁵;
R¹ denotes AR^{X1} or Hetar^{X1};
R⁵ denotes H;
Ar^{X1} denotes phenyl which may be unsubstituted or mono-substituted with R^{X1a} or di-substituted with independently from each other R^{X1a}, R^{X2a};
Hetar^{X1} denotes a bicyclic aromatic ring system with 9 ring atoms wherein (i) 1 of said ring atoms is a nitrogen atom or an oxygen atom or a sulfur atom and the remaining are carbon atoms; or (ii) 1 of said ring atoms is a nitrogen atom and 1 further of said ring atoms is an oxygen atom or a sulfur atom, wherein that further hetero atom may be adjacent or not adjacent to the nitrogen atom, and the remaining are carbon atoms; or (iii) 2 of said ring atoms are nitrogen atoms and the remaining are carbon atoms; or (iv) 2 of said ring atoms are nitrogen atoms and another of said ring atoms is an oxygen atom or a sulfur atom and the remaining are carbon atoms; or (v) 3 of said ring atoms are nitrogen atoms and the remaining are carbon atoms; wherein that aromatic ring system may be unsubstituted or mono-substituted with R^{X1b} or disubstituted with independently from each other R^{X1b}, R^{X2b};
R^{X1a}, R^{X2a} denote independently from each other straight-chain or branched C₁₋₆-alkyl, which C₁₋₆-alkyl may be unsubstituted or mono-, di- or trisubstituted with F and/or Cl, straight-chain or branched O—C₁₋₆-alkyl, which —O—C₁₋₆-alkyl may be unsubstituted or mono-, di- or trisubstituted with F and/or Cl, —OH, —SR^{X9}, —SF₅, F, Cl, Br, —NH₂, —NHR^{X7}, —NR^{X7}R^{X8}, —C(═O)—NH₂, —C(═O)—NHR^{X7}, —C(═O)—NR^{X7}R^{X8} or form together a CH₂—CH₂—O, a —O—CH₂—CH₂—O or a —OCH₂—C(CH₃)₂ chain; if C₁₋₆-alkyl or O—C₁₋₆-alkyl is substituted with one or more F and/or Cl substituents, then it is preferably selected from the group consisting of —CHF₂, —CF₃, —CHF—CHF₂, —OCHF₂, —OCF₃, —OCHF—CHF₂;
R^{X1b}, R^{X2b} denote independently from each other straight-chain or branched C₁₋₆-alkyl, which C₁₋₆-alkyl may be unsubstituted or mono-, di- or trisubstituted with F and/or Cl, Cl, Br, F, —OH, —NH₂, —NHR^{X7}, —NR^{X7}R^{X8}, —NH—C(═O)-methyl, —NH—C(═O)—CH₂—NH₂, —NH—C(═O)-pyrrolidin-2-yl; if C₁₋₆-alkyl is substituted with one or more F and/or Cl substituents, then it is preferably selected from the group consisting of CHF₂, —CF₃, —CHF—CHF₂;
R^{X7}, R^{X8}, R^{X9} denote independently from each other straight-chain or branched C₁₋₆-alkyl or a saturated monocyclic carbocycle with 3, 4, 5, 6, 7 carbon atoms
or
R^{X7} and R^{X8} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl.

In a preferred embodiment, PE2a, of this particular embodiment PE2 the compounds of present invention of formula (I) are those wherein
R² and R³ both denote H (see PE1).

Still another preferred embodiment, PE2b, of this particular embodiment PE2, which may also be part of preferred embodiment PE2a, comprises compounds of formula (I) wherein
R¹ denotes methylphenyl, 3-methylphenyl, ethylphenyl, 3-ethylphenyl, 4-ethylphenyl, trifluoromethyiphenyl, 4-(trifluoromethyl)-phenyl, dimethylphenyl, 2,5-dimethylphenyl, diethylphenyl, 3,5-diethylphenyl, methoxyphenyl, 3-methoxyphenyl, 4-methoxy-phenyl, trifluoromethoxyphenyl, methylsulfanylphenyl, 3-methylsulfanylphenyl, pentafluorosulfanylphenyl, 4-pentafluoro-λ⁶-sulfanylphenyl, 3-trifluoromethoxyphenyl, methoxy-methylphenyl (methoxy-tolyl), 2-methoxy-5-methylphenyl, 5-methoxy-2-methyl-phenyl, fluorophenyl, 4-fluorophenyl, bromophenyl, 3-bromophenyl, 4-bromophenyl, bromo-fluorophenyl, 4-bromo-3-fluoro-phenyl, bromo-methylphenyl, 4-bromo-2-methylphenyl, chloro-methoxyphenyl, 2-chloro-5-methoxy-phenyl, aminophenyl, 3-aminophenyl, 4-aminophenyl, amino-methylphenyl, 2-amino-5-methylphenyl, 3-amino-4-methylphenyl, amino-fluoro-phenyl, 4-amino-3-fluorophenyl, hydroxy-methylphenyl, 2-hydroxy-5-methyl-phenyl, dihydrobenzofuran-5-yl, indolyl, 1H-indol-6-yl, N-methyl-indol-6-yl, 1-ethyl-1H-indol-6-yl(N-ethyl-indol-6-yl), 1-n-propyl-indol-6-yl, N-isopropyl-indol-6-yl, difluoromethyl-indol-6-yl, 2-(difluoromethyl)-1H-indol-6-yl, dimethylindolyl, dimethylindol-6-yl, 1,4-dimethyl-1H-indol-6-yl, 1,5-dimethyl-1H-indol-6-yl, fluoro-methylindolyl, fluoro-1-methylindol-6-yl, 4-fluoro-1-methylindol-6-yl, 5-fluoro-1-methylindol-6-yl, 7-fluoro-1-methyl-indol-6-yl, dimethylaminophenyl, 3-N,N-dimethylaminophenyl, dimethyl-amino-methylphenyl, 2-dimethylamino-5-methylphenyl, benzothiazolyl, benzothiazol-6-yl, benzothiazol-5-yl, dimethyldihydrobenzofuranyl, 3,3-dimethyl-2,3-dihydro-1-benzofuran-5-yl, methylbenzofuranyl, methyl-benzofuran-5-yl, 3-methyl-benzofuran-5-yl, benzothiophenyl, benzothiophen-5-yl, methylbenzothiophenyl, 3-methyl-1-benzothiophen-5-yl, trifluoromethyl-benzothiophenyl, 3-(trifluoromethyl)-1-benzothiophen-5-yl, aminobenzothiophenyl, 2-amino-1-benzothiophen-5-yl, 2-amino-1-benzothiophen-6-yl, 2-(acetylamino)-1-benzothiophen-5-yl, 2-(NH₂—CH₂—C(═O)NH-)-1-benzothiophen-5-yl, 2,3-dihydrobenzo[1,4]dioxin-6-yl, 1-methyl-1H-pyrrolo[2,3-b]pyrdin-6-yl, 1,2-benzothiazol-5-yl, 1,3-benzothiazol-5-yl, 1,3-benzothiazol-6-yl, 2-amino-1,3-benzothiazol-5-yl, 2-methylamino-1,3-benzothiazol-5-yl, 2-dimethylamino-1,3-benzothiazol-5-yl, 2-(acetylamino)-1,3-benzothiazol-5-yl, 2-amino-1,3-benzothiazol-6-yl, 2-(pyrrolidin-2-yl-C(═O)—NH-)-1,3-benzothiazol-5-yl, 2-(pyrrolidin-2-yl-C(═O)—NH-)-1,3-benzothiazol-6-yl, benzothiazololyl (hydroxybenzothiazolyl, dihydro-benzothiazolonyl), 1,3-benzothiazol-2-ol-5-yl (2-hydroxy-1,3-benzothiazol-5-yl, 2,3-dihydro-1,3-benzothiazol-2-on-5-yl), benzoxadiazolyl, 2,1,3-benzoxadiazol-5-yl, benzothiadiazolyl, 2,1,3-benzothiadiazol-5-yl, benzotriazolyl, 1,2,3-benzotriazol-5-yl.

Yet another particular embodiment of the present invention, PE3, comprises compounds of formula (I) wherein
R⁴ denotes Ar^{W4} or Hetar^{W4};
Ar^{W4} denotes phenyl which is substituted with R^{W1a} in the ortho-position (relative to the attachment of Ar^{W4} to X) and may bear no further substituent or one further substituent R^{W2a};
Hetar^{W4} denotes a monocyclic aromatic ring system with 5 or 6 ring atoms wherein 1, 2 or 3 of said ring atoms is/are nitrogen atom(s) and the remaining are carbon atoms, wherein that ring system is substituted with R^{W1b} in the ortho-position (relative to the attachment of Hetar^{W4} to X) and may bear no further substituent or one further substituent R^{W2b};
R^{W1a}, R^{W1b} denote independently from each other LA^{Xa}, Hetar^{X4}, Hetcyc^{X4}, HaI, —CN, —OH, —O—R^{W6a}, —SO₂NH₂, —SO₂NHR^{W4a}, —SO₂NR^{W4a}R^{W6a}, —SO₂—R^{W6a}, —NH₂, —NHR^{W4a}, —NR^{W4a}R^{W6a}, —C(═O)—OH, C(═O)—O—R^{W6a}, —C(═O)—NH₂, —C(═O)—NHR^{W4a}, —C(═O)—NR^{W4a}R^{W5a};
R^{W2a}, R^{W2b} denote independently from each other H, HaI, LA^{Xa}, —CN, —NO₂, —NH₂, —NHR^{W4b}, —NR^{W4b}R^{W6b}, —C(═O)—O—R^{W6b}, —C(═O)—NH₂, —C(═O)—NHR^{W4b}, —C(═O)—NR^{W4b}R^{W5b}, —C(═O)—NH—NH₂, —NH—C(═O)—R^{W6b}, Hetar^{X4}, Hetcyc^{X4};
or R^{W1a} and R^{W2a} or R^{W1b} and R^{W2b} form together a divalent alkylene chain with 3 or 4 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), —O— —wherein that C₁₋₆-alkyl and C₁₋₄-alkyl radicals may be straight-chain or branched —, which divalent alkylene chain may be unsubstituted or mono- or di-substituted with independently from each other straight-chain or branched —C₁₋₆-alkyl;
Ar^{X4} denotes a monocyclic aromatic ring system with 6 ring carbon atoms which ring system may be unsubstituted or monosubstituted with LA^{Xa};
Hetar^{X4} denotes monocyclic aromatic ring system with 5 or 6 ring atoms wherein 1, 2, 3 or 4 of said ring atoms is/are a nitrogen atom(s) and the remaining are carbon atoms, wherein that aromatic ring system may be unsubstituted or mono-substituted with LA^{X4}, —NH₂, —NHR^{X7a}, —NR^{X7a}R^{X8a};
Hetar^{Y4} denotes a monocyclic aromatic ring system with 5 or 6 ring atoms wherein 1, 2 or 3 of said ring atoms is/are a nitrogen atom(s) and the remaining are carbon atoms, wherein that aromatic ring system may be unsubstituted or mono-substituted with LA^{Y4};
Hetcyc^{X4} denotes a saturated mono-cyclic heterocycle with 4, 5 or 6 ring atoms wherein 1 or 2 ring atom(s) is/are heteroatom(s) selected from N, O and/or S and the remaining ring atoms are carbon atoms, wherein that heterocycle may be unsubstituted or monosubstituted with LA^{X4} or C(═O)-LA^{X4} or oxo (═O) or disubstituted with oxo (═O) and LA^{X4} or HaI and LA^{X4} or trisubstituted with one of two HaI and one or two LA^{X4};
Hetcyc^{Y4} denotes a saturated mono-cyclic heterocycle with 4, 5 or 6 ring atoms wherein 1 or 2 ring atom(s) is/are heteroatom(s) selected from N, O and/or S and the remaining ring atoms are carbon atoms, wherein that heterocycle may be unsubstituted or monosubstituted with LA^{Y4} or C(═O)-LA^{Y4} or oxo (═O) or disubstituted with oxo (═O) and LA^{Y4};
LA^{Xa} denotes straight-chain or branched C₁₋₆-alkyl which may be unsubstituted or mono-, di- or trisubstituted with independently from each other HaI, —CN, —NH₂, —NHR^{X7a}, —NR^{X7a}R^{X8a};
LA^{X4} and LA^{Y4} denote independently from each other straight-chain or branched C₁₋₆-alkyl;
LA^{Z4} denotes a straight-chain or branched divalent C₁₋₆-alkylene radical, in particular CH₂;
R^{W4a}, R^{W5a}, R^{W6a}, R^{W4b}, R^{W5b}, R^{W6b} denote independently from each other straight-chain or branched C₁₋₆-alkyl, a saturated monocyclic carbocycle with 3, 4, 5, 6, 7 carbon atoms, Ar^{X4}, Hetar^{X4}, Hetcyc^{X4}, LA^{Z4}-Hetar^{Y4} or LA^{Z4}-Hetcyc^{Y4};
R^{X7a}, R^{X8a} denote independently from each other straight-chain or branched C₁₋₆-alkyl or a saturated monocyclic carbocycle with 3, 4, 5, 6, 7 carbon atoms or a monocyclic aromatic ring system with 5 or 6 ring atoms wherein 1, 2, 3 or 4 of said ring atoms is/are a nitrogen atom(s) and the remaining are carbon atoms, wherein that aromatic ring system may be unsubstituted or mono-substituted with straight-chain or branched C₁₋₆-alkyl;
or
each pair R^{W4a} and R^{W5a}, R^{W4b} and R^{W5b}; R^{X7a} and R^{X8a} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
HaI denotes F, Cl, Br, I.

In a preferred embodiment, PE3a, of said particular embodiment PE3, the compounds of present invention of formula (I) are those with
Ar^{W4} denotes phenyl which is substituted with R^{W1a} in the ortho-position (relative to the attachment of Ar^{W4} to X) and bears no further substituent;
Hetar^{W4} denotes a monocyclic aromatic ring system with 6 ring atoms wherein 1 or 2 of said ring atoms is/are nitrogen atom(s) and the remaining are carbon atoms, wherein that ring system is substituted with R^{W1b} in the ortho-position (relative to the attachment of Hetar^{W4} to X) and bears no further substituent; in particular it denotes a pyridine radical substituted with R^{W1b}.

In still another preferred embodiment, PE3b, of said particular embodiment PE3, the compounds of the present invention of formula (I) are those with
Ar^{W4} denotes phenyl which is substituted with R^{W1a} in the ortho-position (relative to the attachment of Ar^{W4} to X) and bears one further substituent R^{W2a} in para-position relative to R^{W1a};
Hetar^{W4} denotes a monocyclic aromatic ring system with 6 ring atoms wherein 1 or 2 of said ring atoms is/are nitrogen atom(s) and the remaining are carbon atoms, wherein that ring system is substituted with R^{W1b} in the ortho-position (relative to the attachment of Hetar^{W4} to X) and bears one further substituent R^{W2b} in para-position relative to R^{W1b}.

Yet another preferred embodiment, PE3c, of that particular embodiment PE3, comprises compounds of formula (I) with
R^{W1a}, R^{W1b} denote independently from each other methyl, methylaminomethyl, (dimethylamino)methyl, pyrazolyl, methylpyrazolyl, imidazolyl, methylimidazolyl, 1-methyl-1H-imidazol-4-yl, pyrimidinyl, tetrazolyl, 1H-1,2,3,4-tetrazol-5-yl, Cl, —CN, —SO₂NH₂, —SO₂NH(CH₃), —SO₂N(CH₃)₂, —SO₂—N-morpholinyl, —SO₂—N-piperazinyl, —SO₂—CH₃, —SO₂—NH-pyrrolidinyl, —SO₂—NH-pyrrolidin-3-yl, —SO₂—NH-methylpyrrolidinyl, —SO₂—NH-(1-methylpyrrolidin-3-yl), —SO₂—NH-(piperdinyl), —SO₂—NH-(piperdin-3-yl), —SO₂—NH-(methylpiperdinyl), —SO₂—NH-(1-methylpiperdin-3-yl), —SO₂—NH-oxanyl, —SO₂—NH-oxan-3-yl, —SO₂—NH—CH₂-(pyrrolidinyl), —SO₂—NH—CH₂-(pyrrolidin-3-yl), —SO₂—NH—CH₂-(methylpyrrolidinyl), —SO₂—NH—CH₂-(1-methylpyrrolidin-3-yl), —SO₂—NH—CH₂-oxanyl, —SO₂—NH—CH₂-oxan-4-yl, —SO₂—NH—CH₂-pyrazolyl, —SO₂—NH—CH₂-pyrazol-4-yl, —SO₂—NH—CH₂-(methylpyrazolyl), —SO₂—NH—CH₂-(1-methyl-1H-pyrazol-4-yl), —SO₂—NH-(pyrimidin-5-yl), —SO₂—NH—CH₂-(pyrimidin-5-yl), —SO₂—N(CH₃)—CH₂-(pyrimidin-5-yl), —NH₂, —N-piperazinyl, —N-4-methylpiperazinyl, 4-N-acetylpiperazin-1-yl, —OH, —OCH₃, —C(═O)—OH, —C(═O)—O-(n-C₄H₉), —C(═O)—O-pyrimidinyl, —C(═O)—O-pyrimidin-4-yl, —C(═O)—O-(aminopyrimidinyl), —C(═O)—O-(2-aminopyrimidin-4-yl), —C(═O)—NH₂, —C(═O)—NHCH₃, —C(═O)—N(CH₃)₂, —C(═O)—NH-cyclohexyl, —C(═O)—NH-phenyl, —C(═O)—NH-(azetidinyl), —C(═O)—NH-(methylazetidinyl), —C(═O)—NH-(1-methylazetidin-3-yl), —C(═O)—NH-(1-acetylazetidin-3-yl), —C(═O)—NH—CH₂-(azetidinyl), —C(═O)—NH—CH₂-(1-acetylazetidin-3-yl), —C(═O)—NH-(methylpyrrolidinyl), —C(═O)—NH-(1-methyl-pyrrolidin-3-yl), —C(═O)—NH-((3S)-1-methyl-pyrrolidin-3-yl), —C(═O)—NH-((3R)-1-methyl-pyrrolidin-3-yl), —C(═O)—N(CH₃)-(methylpyrrolidinyl), —C(═O)—N(CH₃)-(1-methyl-pyrrolidin-3-yl), —C(═O)—NH—CH₂-(methylpyrrolidinyl), —C(═O)—NH—CH₂-(1-methyl-pyrrolidin-3-yl), —C(═O)—NH-(1-acetylpyrrolidin-3-yl), —C(═O)—NH-(fluoro-methylpyrrolidinyl), —C(═O)—NH-(2-fluoro-1-methyl-pyrrolidin-3-yl), —C(═O)—NH-(5-fluoro-1-methylpyrrolidin-3-yl), —C(═O)—NH-(difluoro-methylpyrrolidinyl), —C(═O)—N H-(5,5-difluoro-1-methylpyrrolidin-3-yl), —C(═O)—NH-(3,3-difluoro-1-methyl-pyrrolidin-3-yl), —C(═O)—NH-oxanyl, —C(═O)—NH-oxan-4-yl, —C(═O)—NH-piperidinyl, —C(═O)—NH-piperidin-4-yl, —C(═O)—NH-piperidin-3-yl, —C(═O)—NH-methylpiperidinyl, —C(═O)—NH-(1-methylpiperidin-4-yl), —C(═O)—NH-(1-methylpiperidin-3-yl), —C(═O)—NH-(acetylpiperdinyl), —C(═O)—NH-(1-acetylpiperidin-3-yl), —C(═O)—NH-(1-acetylpiperidin-4-yl), —C(═O)—NH-(oxopyrrolidinyl), —C(═O)—NH—(N-methyl-oxopyrrolidinyl), —C(═O)—NH-(5-oxopyrrolidin-3-yl), —C(═O)—NH-(2-oxopyrrolidin-3-yl), —C(═O)—NH-(1-methyl-5-oxopyrrolidin-3-yl), —C(═O)—NH-(1-methyl-2-oxopyrrolidin-3-yl), —C(═O)—NH-morpholinyl, —C(═O)—NH—CH₂-morpholinyl, —C(═O)—NH—CH₂-morpholin-2-yl, —C(═O)—NH—CH₂-morpholin-3-yl, —C(═O)—NH—CH₂-(methylmorpholinyl), —C(═O)—NH—CH₂-(4-methylmorpholin-2-yl), —C(═O)—NH—CH₂-(acetylmorpholinyl), —C(═O)—NH—CH₂-(4-acetylmorpholin-2-yl), —C(═O)—NH—CH₂-(4-acetylmorpholin-3-yl), —C(═O)—NH-(oxopiperidinyl), —C(═O)—NH-(2-oxopiperidin-4-yl), —C(═O)—NH-(methyl-oxopiperidinyl), —C(═O)—NH-(1-methyl-2-oxopiperidin-4-yl), —C(═O)—NH-(1-methyl-6-oxopiperidin-3-yl), —C(═O)—NH(pyrimindin-4-yl), —C(═O)—NH(pyrimindin-5-yl), —C(═O)—NHCH₂(pyrimindin-5-yl), —C(═O)—NH-imidazolyl, —C(═O)—NH-imidazol-5-yl, —C(═O)—NH-methylimidazolyl, —C(═O)—NH-(1-methyl-imidazol-5-yl), —C(═O)—NH—CH₂-imidazolyl, —C(═O)—NH—CH₂-imidazol-5-yl, —C(═O)—NH—CH₂-(methylimidazolyl), —C(═O)—NH—CH₂-(1-methyl-1H-imidazol-5-yl), —C(═O)—NH(methyl-pyrazolyl), —C(═O)—NH(1-methyl-1H-pyrazol-4-yl), —C(═O)—NHCH₂(1-methylpyrazol-4-yl), —C(═O)—NH₂-pyridinyl, —C(═O)—NH₂-pyridin-3-yl, —C(═O)—NH-pyridazinyl, —C(═O)—NH-pyridazin-3-yl, —C(═O)—NH—CH₂-pyridazinyl, —C(═O)—NH-pyrimidinyl, —C(═O)—NH-pyrimidin-4-yl, —C(═O)—NH-pyrimidin-5-yl, —C(═O)—NH—CH₂-pyridazin-3-yl, —CH₂—NH-(pyrimidin-5-yl);
R^{W2a}, R^{W2b} denote, if present, independently from each other H, Br, —CH₂NH₂, —CN, —NO₂, —NH₂, —NH—C(═O)—CH₃, —C(═O)—O-methyl, —C(═O)—NH₂, —C(═O)—NH—NH₂, 4-methylpiperazin-1-yl, 4-acetyl-piperazin-1-yl, methylpyrazolyl, 1-methyl-1H-pyrazol-5-yl, 1H-imidazol-1-yl, oxazolyl, 1,3-oxazol-2-yl, 2H-1,2,3,4-tetrazol-5-yl;
or R^{W1b} and R^{W2b} form together a divalent —O—CH₂—CH₂—NH— chain it being understood that the the oxygen atom of that chain is attached to the Hetar^{W4} substituent at the position of R^{W1b} while the —NH— part of that chain is attached to the Hetar^{W4} substituent at the position of R^{W2b} and next to R^{W1b}.

Another preferred embodiment, PE3d, is a combination of preferred embodiment PE3a with preferred embodiment PE3c. Yet another preferred embodiment, PE3e, is a combination of preferred embodiment PE3b with preferred embodiment PE3c.

Still another preferred embodiment, PE3f, of the particular embodiment PE3 and optionally of preferred embodiments PE3a, PE3b, PE3c, PE3d, and PE3e, comprises compounds of formula (I) wherein
Ar^{W4} denotes 2-((dimethylamino)methyl)phenyl, 2-(C(═O)OH)phenyl, 2-methylsulfonylphenyl (2-methanesulfonylphenyl), 2-(morpholine-4-sulfonyl)phenyl, 2-hydroxyphenyl, 2-methoxy-phenyl, 2-cyanophenyl, 2-aminosulfonylphenyl, 2-(N-methyl-aminosulfonyl)phenyl, 2-((1-methylpyrrolidin-3-yl)-NH—SO₂-)phenyl, 2-((1-methylpiperidin-3-yl)-NH—SO₂-)phenyl, 2-((oxan-3-yl)-NH—SO₂-)phenyl, 2-((1-methylpyrrolidin-3-yl)-CH₂—NH—SO₂-)phenyl, 2-(oxan-4-yl-CH₂—NH—SO₂-)phenyl, 2-((1-methyl-1H-pyrazol-4-yl)-CH₂—NH—SO₂-)phenyl, 2-((pyrimidin-5-yl)-CH₂—NH—SO₂-)phenyl, 2-((pyrimidin-5-yl)-CH₂—N(CH₃)—SO₂-)phenyl, 2-(N,N-dimethylaminosulfonyl)phenyl, 2-(NH₂—C(═O)-)phenyl (2-carbarnoylphenyl), 2-((1-methylpyrrolidin-3-yl)-NH—C(═O)-)phenyl, 5-bromo-2-methanesulfonylphenyl, 2-(piperazine-1-sulfonyl)-phenyl, 5-cyano-2-methanesulfonylphenyl, 2-methanesulfonyl-5-amino-phenyl, 2-methanesulfonyl-5-nitro-phenyl, 2-methane-sulfonyl-5-aminomethyl-phenyl, 2-methanesulfonyl-5-carbamoyl-phenyl (2-methanesulfonyl-5-(NH₂—C(═O)-)phenyl), (2-methane-sulfonyl-5-(NH₂—NH—C(═O)-)phenyl), 2-methanesulfonyl-5-(CH₃C(═O)NH)-phenyl, 2-methanesulfonyl-5-(4-acetylpiperazin-1-yl)-phenyl, 2-methanesulfonyl-5-(4-methylpiperazin-1-yl)-phenyl, 2-methanesulfonyl-5-(1,3-oxazol-2-yl)phenyl, methane-sulfonyl-5-(2H-1,2,3,4-tetrazol-5-yl)phenyl, 5-(1H-imidazol-1-yl)-2-methanesulfonylphenyl;
Hetar^{W4} denotes 4-(methylamino)methylpyridin-3-yl, 4-((dimethyl-amino)methyl)pyridin-3-yl, 2-methylsulfonylpyrdin-3-yl, 4-methyl-sulfonylpyridin-3-yl, 2-aminopyridin-3-yl, 4-(NH₂—C(═O))-pyridin-3-yl, 4-chloropyridin-3-yl, 4-cyanopyridin-3-yl, 2-hydroxy-pyridin-3-yl, 2-methoxy-pyridin-3-yl, 3-methanesulfonyl-pyrazin-2-yl, 3-methanesulfonyl-pyridin-2-yl, 4-(C(═O)OH)pyridin-3-yl, 4-(1-methyl-1H-pyrazol-4-yl)-pyridin-3-yl, 4-(4-methylpiperazin-1-yl)-pyridin-3-yl, 4-(4-N-acetylpiperazin-1-yl)pyridin-3-yl, 4-(1-methyl-1H-imidazol-4-yl)pyridin-3-yl, 4-(pyrimidin-5-yl)-pyridin-3-yl, 4-methoxypyridin-3-yl, 4-(CH₃NH—C(═O))-pyridin-3-yl, 4-(1H-1,2,3,4-tetrazol-5-yl)pyridin-3-yl, 4-((2-aminopyrimidin-4-yl)-O—C(═O))-pyridin-3-yl, 4-((CH₃)₂N—C(═O))-pyridin-3-yl, 4-((-(1-methylazetidin-3-yl)-NH—C(═O)-)pyridin-3-yl, 4-((1-acetylazetidin-3-yl)-NH—C(═O)-)pyridin-3-yl, 4-((1-methylpyrrolidin-3-yl)-NH—C(═O)-)pyridin-3-yl (4-(1-methylpyrrolidin-3-ylcarbamoyl)pyridin-3-yl), 4-((1-methylpyrrolidin-3-yl)-N(CH₃)—C(═O)-)pyridin-3-yl, 4-(1-methyl-pyrrolidin-3-yl)-CH₂—NH—C(═O)-pyridin-3-yl (4-(1-methyl-pyrrolidin-3-ylmethylcarbamoyl)pyridin-3-yl), 4-(1-acetyl-pyrrolidin-3-yl)-NH—C(═O)-pyridin-3-yl, 4-(oxan-4-yl-NH—C(═O))pyridin-3-yl, 4-((1-methylpiperidin-4-yl)-NH—C(═O)-)pyridin-3-yl(4-(1-methylpiperidin-4-ylcarbamoyl)pyridin-3-yl), 4-((1-methylpiperidin-3-yl)-NH—C(═O)-)pyridin-3-yl(4-(1-methyl-piperidin-3-ylcarbamoyl)pyridin-3-yl), 4-(((3S)-1-methyl-pyrrolidin-3-yl)-NH—C(═O)-)pyridin-3-yl, 4-(((3R)-1-methyl-pyrrolidin-3-yl)-NH—C(═O)-)pyridin-3-yl, 4-(5-fluoro-1-methylpyrrolidin-3-yl)-NH—C(═O)-pyridin-3-yl, 4-(3-fluoro-1-methylpyrrolidin-3-yl)-NH—C(═O)-pyridin-3-yl, 4-(5,5-difluoro-1-methylpyrrolidin-3-yl)-NH—C(═O)-pyridin-3-yl, 4-(3,3-difluoro-1-methylpyrrolidin-3-yl)-NH—C(═O)-pyridin-3-yl, 4-(1-acetylpiperidin-3-ylcarbamoyl)pyridin-3-yl, 4-(1-acetylpiperidin-4-ylcarbamoyl)pyridin-3-yl, 4-(1-acetylpiperidin-3-ylmethylcarbamoyl)pyridin-3-yl, 4-(1-acetylpiperidin-4-ylmethyl-carbamoyl)pyridin-3-yl, 4-((1-acetylazetidin-3-yl)-CH₂—NH—C(═O)-) pyridin-3-yl(4-(1-acetylazetidin-3-ylmethylcarbamoyl)pyridin-3-yl), 4-(5-oxopyrrolidin-3-yl)-NH—C(═O)-pyridin-3-yl, 4-(2-oxo-pyrrolidin-3-yl)-N H—C(═O)-pyridin-3-yl, 4-(1-methyl-5-oxopyrrolidin-3-yl)-NH—C(═O)-pyridin-3-yl, 4-(1-methyl-2-oxopyrrolidin-3-yl)-NH—C(═O)-pyridin-3-yl, 4-(morpholin-3-yl)-CH₂—NH—C(═O)-pyridin-3-yl, 4-(4-methylmorpholin-2-yl)-CH₂—NH—CO-pyridin-3-yl, (4-acetylmorpholin-3-yl)-CH₂—NH—C(═O)-pyridin-3-yl, 4-acetyl-morpholin-2-yl-CH₂—NH—C(═O)-pyridin-3-yl(4-acetylmorpholin-2-ylmethylcarbamoylpyridin-3-yl), 4-((2-oxopiperidin-4-yl)-NH—C(═O)-)pyridin-3-yl(4-(2-oxopiperidin-4-ylcarbamoyl)pyridin-3-yl), 4-((1-methyl-2-oxopiperidin-4-yl)-NH—C(═O)-)pyridin-3-yl(4-(1-methyl-2-oxopiperidin-4-ylcarbamoyl)pyridin-3-yl), 4-(1-methyl-6-oxopiperidin-3-yl)-NH—C(═O)-)pyridin-3-yl(4-(1-methyl-6-oxo-piperidin-3-ylcarbamoyl)pyridin-3-yl, 4-(phenyl-NH—C(═O)-)-pyridin-3-yl (4-(phenylcarbamoyl)pyridin-3-yl), 4-((1-methyl-1H-pyrazol-4-yl)NH—C(═O))pyridin-3-yl, 4-((1-methylpyrazol-4-yl)-CH₂NH—C(═O))-pyridin-3-yl, 4-(pyridine-3-yl)-NH—C(═O)-pyridin-4-yl, 4-((1-methyl-imidazol-5-yl)-CH₂—NH—C(═O)-)pyridin-3-yl) (4-(1-methyl-imidazol-5-ylmethyl)carbamoylpyridin-3-yl), 4-((pyrimidin-4-yl)-NH—C(═O))pyridin-3-yl, 4-((pyrimidinyl-5-yl)-NHC(═O))-pyridin-3-yl, 4-((pyrimidinyl-5-yl)-CH₂NHC(═O))-pyridin-3-yl, 4-(pyridazin-3-ylmethylcarbamoyl)pyridin-3-yl, 4-methanesulfonyl-pyridin-1-ium-1-olate-3-yl, 2H,3H,4H-pyrido[4,3-b][1,4]oxazin-8-yl, 4-carbamoylpyrimidin-5-yl, 1-methyl-1H-1,2,3-triazol-5-yl, 4-[(pyrimidin-5-yl)amino]methylpyridin-3-yl.

Another particular embodiment, PE4, of the present invention is a combination of particular embodiment PE2 or its preferred embodiments PE2a or PE2b with particular embodiment PE3 or its preferred embodiments PE3a, PE3b, PE3c, PE3d, PE3e, PE3f. A preferred embodiment, PE4a, of said particular embodiment PE4 comprises compounds of formula (I) wherein
R¹ denotes 4-ethylphenyl, 2,5-dimethyiphenyl, 3-methoxyphenyl, 4-fluorophenyl, 3-bromophenyl, 4-bromophenyl, 2-chloro-5-methoxy-phenyl, 3-amino-4-methylphenyl, 4-amino-3-fluoro-phenyl, dihydrobenzofuran-5-yl, N-methyl-indol-6-yl, 1-ethyl-1H-indol-6-yl, 2-(difluoromethyl)-1H-indol-6-yl, 1,4-dimethyl-1H-indol-6-yl, 1,5-dimethyl-1H-indol-6-yl, 4-fluoro-1-methylindol-6-yl, 5-fluoro-1-methylindol-6-yl, 7-fluoro-1-methyl-indol-6-yl, benzothiazol-6-yl, benzothiazol-5-yl, 3-methyl-benzofuran-5-yl, 3-methyl-1-benzothiophen-5-yl, 2,3-dihydrobenzo[1,4]dioxin-6-yl, 1-methyl-1H-pyrrolo[2,3-b]pyrdin-6-yl, 2-amino-1,3-benzothiazol-5-yl, 2-amino-1,3-benzothiazol-6-yl, 2-(pyrrolidin-2-yl-C(═O)—NH-)-1,3-benzothiazol-6-yl, 2,1,3-benzothiadiazol-5-yl; R⁴ denotes 2-methylsulfonylphenyl, 2-((dimethylamino)methyl)-phenyl, 2-(C(═O)OH)phenyl, 2-methylsulfonylphenyl (2-methanesulfonylphenyl), 2-(morpholine-4-sulfonyl)phenyl, 2-hydroxyphenyl, 2-methoxyphenyl, 2-cyanophenyl, 2-amino-sulfonylphenyl, 2-(N-methylaminosulfonyl)phenyl, 2-((1-methyl-pyrrolidin-3-yl)-NH—SO₂-)phenyl, 2-((1-methylpiperidin-3-yl)-NH—SO₂-)phenyl, 2-((oxan-3-yl)-NH—SO₂-)phenyl, 2-((1-methyl-pyrrolidin-3-yl)-CH₂—NH—SO₂-)phenyl, 2-(oxan-4-yl-CH₂—NH—SO₂-) phenyl, 2-((1-methyl-1H-pyrazol-4-yl)-CH₂—NH—SO₂-)phenyl, 2-((pyrimidin-5-yl)-CH₂—NH—SO₂-)phenyl, 2-((pyrimidin-5-yl)-CH₂-2-(N,N-dimethylaminosulfonyl)phenyl, 2-(NH₂—C(═O)-)phenyl (2-carbamoylphenyl), 2-((1-methylpyrrolidin-3-yl)-NH—C(═O)-)phenyl, 5-bromo-2-methanesulfonylphenyl, 2-(piperazine-1-sulfonyl)phenyl, 5-cyano-2-methanesulfonylphenyl, 2-methansulfonyl-5-amino-phenyl, 2-methansulfonyl-5-nitro-phenyl, 2-methansulfonyl-5-aminomethyl-phenyl, 2-methane-sulfonyl-5-carbamoylphenyl (2-methanesulfonyl-5-(NH₂—C(═O)-) phenyl), (2-methanesulfonyl-5-(NH₂—NH—C(═O)-)phenyl), 2-methansulfonyl-5-(CH₃C(═O)NH)-phenyl, 2-methanesulfonyl-5-(4-acetylpiperazin-1-yl)-phenyl, 2-methanesulfonyl-5-(4-methyl-piperazin-1-yl)-phenyl, 2-methanesulfonyl-5-(1,3-oxazol-2-yl)-phenyl, methanesulfonyl-5-(2H-1,2,3,4-tetrazol-5-yl)phenyl, 5-(1H-imidazol-1-yl)-2-methanesulfonylphenyl, 4-(methylamino)-methylpyridin-3-yl, 4-((dimethylamino)methyl)pyridin-3-yl, 2-methylsulfonylpyrdin-3-yl, 4-methylsulfonylpyridin-3-yl, 2-amino-pyridin-3-yl, 4-(NH₂—C(═O))-pyridin-3-yl, 4-chloropyridin-3-yl, 4-cyanopyridin-3-yl, 2-hydroxy-pyridin-3-yl, 2-methoxy-pyridin-3-yl, 3-methanesulfonyl-pyrazin-2-yl, 3-methanesulfonyl-pyridin-2-yl, 4-(C(═O)OH)pyridin-3-yl, 4-(1-methyl-1H-pyrazol-4-yl)-pyridin-3-yl, 4-(4-methylpiperazin-1-yl)-pyridin-3-yl, 4-(4-N-acetylpiperazin-1-yl)pyridin-3-yl, 4-(1-methyl-1H-imidazol-4-yl)pyridin-3-yl, 4-(pyrimidin-5-yl)-pyridin-3-yl, 4-methoxypyridin-3-yl, 4-(1H-1,2,3,4-tetrazol-5-yl)pyridin-3-yl, 4-((2-aminopyrimidin-4-yl)-O—C(═O))-pyridin-3-yl, 4-(CH₃NH—C(═O))-pyridin-3-yl, 4-((CH₃)₂N—C(═O))-pyridin-3-yl, 4-((-(1-methylazetidin-3-yl)-NH—C(═O)-)pyridin-3-yl, 4-((1-acetylazetidin-3-yl)-NH—C(═O)-)pyridin-3-yl, 4-((1-methyl-pyrrolidin-3-yl)-NH—C(═O)-)pyridin-3-yl (4-(1-methylpyrrolidin-3-ylcarbamoyl)pyridin-3-yl), 4-((1-methylpyrrolidin-3-yl)-N(CH₃)—C(═O)-)pyridin-3-yl, 4-(1-methyl-pyrrolidin-3-yl)-CH₂—NH—C(═O)-pyridin-3-yl (4-(1-methyl-pyrrolidin-3-ylmethylcarbamoyl)pyridin-3-yl), 4-(1-acetylpyrrolidin-3-yl)-NH—C(═O)-pyridin-3-yl, 4-(oxan-4-yl-NH—C(═O))pyridin-3-yl, 4-((1-methylpiperidin-4-yl)-NH—C(═O)-) pyridin-3-yl(4-(1-methylpiperidin-4-ylcarbamoyl)pyridin-3-yl), 4-((1-methylpiperidin-3-yl)-NH—C(═O)-)pyridin-3-yl(4-(1-methyl-piperidin-3-ylcarbamoyl)pyridin-3-yl), 4-(((3S)-1-methyl-pyrrolidin-3-yl)-NH—C(═O)-)pyridin-3-yl, 4-(((3R)-1-methyl-pyrrolidin-3-yl)-NH—C(═O)-)pyridin-3-yl, 4-(1-acetylpiperidin-3-ylcarbamoyl)-pyridin-3-yl, 4-(1-acetylpiperidin-4-ylcarbamoyl)pyridin-3-yl, 4-(1-acetylpiperidin-3-ylmethylcarbamoyl)pyridin-3-yl, 4-(1-acetyl-piperidin-4-ylmethylcarbamoyl)pyridin-3-yl, 4-((1-acetylazetidin-3-yl)-CH₂—NH—C(═O)-)pyridin-3-yl(4-(1-acetylazetidin-3-ylmethyl-carbamoyl)pyridin-3-yl), 4-(morpholin-3-yl)-CH₂—NH—C(═O)-pyridin-3-yl, 4-(4-methylmorpholin-2-yl)-CH₂—NH—CO-pyridin-3-yl, (4-acetylmorpholin-3-yl)-CH₂—NH—C(═O)-pyridin-3-yl, 4-acetyl-morpholin-2-yl-CH₂—NH—C(═O)-pyridin-3-yl(4-acetylmorpholin-2-ylmethylcarbamoylpyridin-3-yl), 4-((2-oxopiperidin-4-yl)-NH—C(═O)-)pyridin-3-yl(4-(2-oxopiperidin-4-ylcarbamoyl)pyridin-3-yl), 4-((1-methyl-2-oxopiperidin-4-yl)-NH—C(═O)-)pyridin-3-yl(4-(1-methyl-2-oxopiperidin-4-ylcarbamoyl)pyridin-3-yl), 4-(1-methyl-6-oxopiperidin-3-yl)-NH—C(═O)-)pyridin-3-yl(4-(1-methyl-6-oxo-piperidin-3-ylcarbamoyl)pyridin-3-yl, 4-(phenyl-NH—C(═O)-)-pyridin-3-yl(4-(phenylcarbamoyl)pyridin-3-yl), 4-((1-methyl-1H-pyrazol-4-yl)NH—C(═O))pyridin-3-yl, 4-((1-methylpyrazol-4-yl)-CH₂NH—C(═O))-pyridin-3-yl, 4-((1-methyl-imidazol-5-yl)-CH₂—NH—C(═O)-)pyridin-3-yl) (4-(1-methyl-imidazol-5-ylmethyl)carbamoyl-pyridin-3-yl), 4-((pyrimidinyl-5-yl)-NHC(═O))-pyridin-3-yl, 4-((pyrimidinyl-5-yl)-CH₂N HC(═O))-pyridin-3-yl, 4-(pyridazin-3-ylmethylcarbamoyl)pyridin-3-yl, 4-methanesulfonyl-pyridin-1-ium-1-olate-3-yl, 2H,3H,4H-pyrido[4,3-b][1,4]oxazin-8-yl, 4-carbamoylpyrimidin-5-yl, 1-methyl-1H-1,2,3-triazol-5-yl, 4-[(pyrimidin-5-yl)amino]methylpyridin-3-yl.

In yet another preferred embodiment, PE4b, of that preferred embodiment PE4a, the substituents R² and R³ of the compounds of formula (I) are both hydrogen.

It is still another particular embodiment, PE5, of the present invention, that comprises a compound selected from the following group, N-oxides thereof and physiologically acceptable salts either of the compound or any of its N-oxides, the group consisting of:
8-(2,3-dihydro-1,4-benzodioxin-6-yl)-N-(4-methanesulfonylpyridin-3-yl)quinoxalin-6-amine
5-(1-methyl-1H-indol-6-yl)-7-{1H,2H,3H-pyrrolo[2,3-c]pyridin-1-yl}quinoxaline
N-(2-methanesulfonylphenyl)-8-(1-methyl-1H-indol-6-yl)quinoxalin-6-amine
8-(1,3-benzothiazol-6-yl)-N-(4-methanesulfonylpyridin-3-yl)quinoxalin-6-amine
8-(2-chloro-5-methoxyphenyl)-N-(4-methanesulfonylpyridin-3-yl)quinoxalin-6-amine
N-(2-methanesulfonylpyridin-3-yl)-8-(1-methyl-1H-indol-6-yl)quinoxalin-6-amine
8-(1-methyl-1H-indol-6-yl)-N-[2-(morpholine-4-sulfonyl)phenyl]quinoxalin-6-amine
2-{[8-(1-methyl-1H-indol-6-yl)quinoxalin-6-yl]amino}benzene-1-sulfonamide
8-(1,3-benzothiazol-5-yl)-N-(4-methanesulfonylpyridin-3-yl)quinoxalin-6-amine trifluoroacetate
N-(5-bromo-2-methanesulfonylphenyl)-8-(1-methyl-1H-indol-6-yl)quinoxalin-6-amine
N-(4-methanesulfonylpyridin-3-yl)-8-(1-methyl-1H-indol-6-yl)quinoxalin-6-amine
N-(2-methoxypyridin-3-yl)-8-(1-methyl-1H-indol-6-yl)quinoxalin-6-amine
3-{[8-(1-methyl-1H-indol-6-yl)quinoxalin-6-yl]amino}pyridin-2-ol
8-(1-methyl-1H-indol-6-yl)-N-[2-(piperazine-1-sulfonyl)phenyl]quinoxalin-6-amine
N-methyl-2-{[8-(1-methyl-1H-indol-6-yl)quinoxalin-6-yl]amino}benzene-1-sulfonamide
3-N-[8-(1-methyl-1H-indol-6-yl)quinoxalin-6-yl]pyridine-2,3-diamine
3-{[8-(1-methyl-1H-indol-6-yl)quinoxalin-6-yl]amino}pyridine-4-carbonitrile
3-{[8-(1-methyl-1H-indol-6-yl)quinoxalin-6-yl]amino}pyridine-4-carboxamide
N,N-dimethyl-2-{[8-(1-methyl-1H-indol-6-yl)quinoxalin-6-yl]amino}benzene-1-sulfonamide
N-(2-methanesulfonylphenyl)-8-{1-methyl-1H-pyrrolo[2,3-b]pyridin-6-yl}quinoxalin-6-amine trifluoroacetate
N-(4-methanesulfonylpyridin-3-yl)-8-(3-methyl-1-benzofuran-5-yl)quinoxalin-6-amine
N-(4-methoxypyridin-3-yl)-8-(1-methyl-1H-indol-6-yl)quinoxalin-6-amine
3-{[8-(3-methyl-1-benzofuran-5-yl)quinoxalin-6-yl]amino}pyridine-4-carbonitrile
4-methanesulfonyl-3-{[8-(1-methyl-1H-indol-6-yl)quinoxalin-6-yl]amino}benzonitrile
3-{[8-(3-methyl-1-benzofuran-5-yl)quinoxalin-6-yl]amino}pyridine-4-carboxamide
N-(5-methanesulfonylpyrimidin-4-yl)-8-(1-methyl-1H-indol-6-yl)quinoxalin-6-amine
3-{[8-(1-methyl-1H-indol-5-yl)quinoxalin-6-yl]amino}pyridine-4-carbonitrile
3-{[8-(1-methyl-1H-indol-5-yl)quinoxalin-6-yl]amino}pyridine-4-carboxamide
N-(4-chloropyridin-3-yl)-8-(1-methyl-1H-indol-5-yl)quinoxalin-6-amine
8-(1-methyl-1H-indol-5-yl)-N-[4-(1-methyl-1H-pyrazol-4-yl)pyridin-3-yl]quinoxalin-6-amine
8-(1-methyl-1H-indol-5-yl)-N-[4-(4-methylpiperazin-1-yl)pyridin-3-yl]quinoxalin-6-amine
8-(1-methyl-1H-indol-5-yl)-N-[4-(pyrimidin-5-yl)pyridin-3-yl]quinoxalin-6-amine
5-(1-methyl-1H-indol-5-yl)-7-{1H,2H,3H-pyrrolo[2,3-c]pyridin-1-yl}quinoxaline
N-(2-methanesulfonyl-5-nitrophenyl)-8-(1-methylindol-6-yl)quinoxalin-6-amine
6-methanesulfonyl-N1-[8-(1-methyl-1H-indol-6-yl)quinoxalin-6-yl]benzene-1,3-diamine
8-(2,3-dihydro-1-benzofuran-5-yl)-N-(4-methanesulfonylpyridin-3-yl)quinoxalin-6-amine
N-[5-(aminomethyl)-2-methanesulfonylphenyl]-8-(1-methyl-1H-indol-5-yl)quinoxalin-6-amine
8-(2,5-dimethylphenyl)-N-(4-methanesulfonylpyridin-3-yl)quinoxalin-6-amine
8-(1-methyl-1H-indol-6-yl)-N-[4-(4-methylpiperazin-1-yl)pyridin-3-yl]quinoxalin-6-amine
N-(4-methanesulfonyl-3-{[8-(1-methyl-1H-indol-6-yl) quinoxalin-6-yl]amino}phenyl)acetamide
N-[5-(1H-imidazol-1-yl)-2-methanesulfonylphenyl]-8-(1-methyl-1H-indol-6-yl)quinoxalin-6-amine
N-[2-methanesulfonyl-5-(2H-1,2,3,4-tetrazol-5-yl)phenyl]-8-(1-methyl-1H-indol-6-yl)quinoxalin-6-amine
4-methanesulfonyl-3-{[8-(1-methyl-1H-indol-6-yl)quinoxalin-6-yl]amino}pyridin-1-ium-1-olate
N-[2-methanesulfonyl-5-(4-methylpiperazin-1-yl)phenyl]-8-(1-methyl-1H-indol-6-yl)quinoxalin-6-amine
1-[4-(4-methanesulfonyl-3-{[8-(1-methyl-1H-indol-6-yl)quinoxalin-6-yl]amino}phenyl)piperazin-1-yl]ethan-1-one
3-{[8-(1-methyl-1H-indol-6-yl)quinoxalin-6-yl]oxy}pyridine-4-carbonitrile
N-(4-methanesulfonylpyridin-3-yl)-8-[3-(1H-1,2,3-triazol-4-yl)phenyl]quinoxalin-6-amine
N-(4-methanesulfonylpyridin-3-yl)-8-[1-(propan-2-yl)-1H-indol-6-yl]quinoxalin-6-amine
8-[3-(dimethylamino)phenyl]-N-(4-methanesulfonylpyridin-3-yl)quinoxalin-6-amine
N-(4-methanesulfonylpyridin-3-yl)-8-(3-methylphenyl)quinoxalin-6-amine
N-methyl-3-{[8-(1-methyl-1H-indol-6-yl)quinoxalin-6-yl]amino}pyridine-4-carboxamide
N,N-dimethyl-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-(pyrimidin-5-yl)pyridine-4-carboxamide
3-{[8-(1-methyl-1H-indol-6-yl)quinoxalin-6-yl]amino}-N-(pyrimidin-5-ylmethyl)pyridine-4-carboxamide
3-{[8-(1-methyl-1H-indol-6-yl)quinoxalin-6-yl]amino}-N-[(1-methyl-1H-pyrazol-4-yl)methyl]pyridine-4-carboxamide
4-methanesulfonyl-N1-methyl-N3-[8-(1-methyl-1H-indol-6-yl)quinoxalin-6-yl]benzene-1,3-diamine
8-[3-(chloromethyl)-1-benzofuran-5-yl]-N-(4-methanesulfonylpyridin-3-yl)quinoxalin-6-amine
8-(7-fluoro-1-methyl-1H-indol-6-yl)-N-(4-methanesulfonylpyridin-3-yl)quinoxalin-6-amine
8-(4-ethylphenyl)-N-(4-methanesulfonylpyridin-3-yl)quinoxalin-6-amine
8-(1H-1,3-benzodiazol-5-yl)-N-(4-methanesulfonylpyridin-3-yl)quinoxalin-6-amine
N-(4-methanesulfonylpyridin-3-yl)-8-(3-methoxyphenyl)quinoxalin-6-amine
8-(3,3-dimethyl-2,3-dihydro-1-benzofuran-5-yl)-N-(4-methanesulfonylpyridin-3-yl)quinoxalin-6-amine
8-(3-ethylphenyl)-N-(4-methanesulfonylpyridin-3-yl)quinoxalin-6-amine
8-(2-amino-5-methylphenyl)-N-(4-methanesulfonylpyridin-3-yl)quinoxalin-6-amine
2-{7-[(4-methanesulfonylpyridin-3-yl)amino]quinoxalin-5-yl}-4-methylphenol
8-(1-methyl-1H-indol-6-yl)-N-[4-(1H-1,2,3,4-tetrazol-5-yl)pyridin-3-yl]quinoxalin-6-amine
N-(4-chloropyridin-3-yl)-8-(1-methyl-1H-indol-6-yl)quinoxalin-6-amine
8-(4-fluoro-1-methyl-1H-indol-6-yl)-N-(4-methanesulfonylpyridin-3-yl)quinoxalin-6-amine
4-methanesulfonyl-3-{[8-(3-methyl-1-benzofuran-5-yl)quinoxalin-6-yl]amino}pyridin-1-ium-1-olate
8-(5-fluoro-1-methyl-1H-indol-6-yl)-N-(4-methanesulfonylpyridin-3-yl)quinoxalin-6-amine
N-(4-methanesulfonylpyridin-3-yl)-8-(2-methoxy-5-methylphenyl)quinoxalin-6-amine
8-(3-amino-4-methylphenyl)-N-(4-methanesulfonylpyridin-3-yl)quinoxalin-6-amine
N-(3-methanesulfonylpyridin-2-yl)-8-(1-methyl-1H-indol-6-yl)quinoxalin-6-amine
1-[4-(3-{[8-(1-methyl-1H-indol-6-yl)quinoxalin-6-yl]amino}pyridin-4-yl)piperazin-1-yl]ethan-1-one
N-[4-(1-methyl-1H-imidazol-4-yl)pyridin-3-yl]-8-(1-methyl-1H-indol-6-yl)quinoxalin-6-amine
8-(1-methyl-1H-indol-6-yl)-N-{2H,3H,4H-pyrido[4,3-b][1,4]oxazin-8-yl}quinoxalin-6-amine
2-{[8-(1-methyl-1H-indol-6-yl)quinoxalin-6-yl]amino}-N-[(pyrimidin-5-yl)methyl]benzene-1-sulfonamide
2-{[8-(1-methyl-1H-indol-6-yl)quinoxalin-6-yl]amino}benzonitrile
2-{[8-(1-methyl-1H-indol-6-yl)quinoxalin-6-yl]amino}benzamide
4-cyano-3-{[8-(1-methyl-1H-indol-6-yl)quinoxalin-6-yl]amino}pyridin-1-ium-1-olate
3-{methyl[8-(1-methyl-1H-indol-6-yl)quinoxalin-6-yl]amino}pyridine-4-carbonitrile
3-{[8-(1-methyl-1H-indol-6-yl)quinoxalin-6-yl]amino}-N-(1-methyl-1H-pyrazol-4-yl)pyridine-4-carboxamide
N-[2-methanesulfonyl-5-(1-methyl-1H-pyrazol-5-yl)phenyl]-8-(1-methyl-1H-indol-6-yl)quinoxalin-6-amine
N-[2-methanesulfonyl-5-(1,3-oxazol-2-yl)phenyl]-8-(1-methyl-1H-indol-6-yl)quinoxalin-6-amine
3-{methyl[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-phenylpyridine-4-carboxamide
3-{[8-(1-methyl-1H-indol-6-yl)quinoxalin-6-yl]amino}-N-(1-methyl-2-oxopiperidin-4-yl)pyridine-4-carboxamide
N-(1-acetylazetidin-3-yl)-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-(1-methylpyrrolidin-3-yl)pyridine-4-carboxamide
2-{[8-(1-methyl-1H-indol-6-yl)quinoxalin-6-yl]amino}-N-(pyrimidin-5-yl)benzene-1-sulfonamide
3-{[8-(1-methyl-1H-indol-6-yl)quinoxalin-6-yl]amino}-N-(oxan-4-yl)pyridine-4-carboxamide
6-methanesulfonyl-N1-[8-(3-methyl-1-benzofuran-5-yl)quinoxalin-6-yl]benzene-1,3-diamine
N-(2-methanesulfonyl-5-nitrophenyl)-8-(3-methyl-1-benzofuran-5-yl)quinoxalin-6-amine
N-(4-methanesulfonylpyridin-3-yl)-N-methyl-8-(1-methyl-1H-indol-6-yl)quinoxalin-6-amine
N-(4-methanesulfonylpyridin-3-yl)-8-(3-methyl-1-benzothiophen-5-yl)quinoxalin-6-amine
Methyl 4-methanesulfonyl-3-{[8-(1-methyl-1H-indol-6-yl)quinoxalin-6-yl]amino}benzoate
4-Methanesulfonyl-3-{[8-(1-methyl-1H-indol-6-yl)quinoxalin-6-yl]amino}benzamide
8-(2,1,3-benzothiadiazol-5-yl)-N-(4-methanesulfonylpyridin-3-yl)quinoxalin-6-amine
8-(1H-1,2,3-benzotriazol-5-yl)-N-(4-methanesulfonylpyridin-3-yl)quinoxalin-6-amine
4-methanesulfonyl-3-{[8-(1-methyl-1H-indol-6-yl)quinoxalin-6-yl]amino}benzohydrazide
8-(2,1,3-benzoxadiazol-5-yl)-N-(4-methanesulfonylpyridin-3-yl)quinoxalin-6-amine
N-(1-acetylpyrrolidin-3-yl)-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-(1-methyl-6-oxopiperidin-3-yl)pyridine-4-carboxamide
3-{[8-(1-methyl-1H-indol-6-yl)quinoxalin-6-yl]amino}-N-(1-methylpiperidin-4-yl)pyridine-4-carboxamide
3-{[8-(1-methyl-1H-indol-6-yl)quinoxalin-6-yl]amino}-N-(1-methylpiperidin-3-yl)pyridine-4-carboxamide
3-{methyl[8-(1-methyl-1H-indol-6-yl)quinoxalin-6-yl]amino}-N-(pyrimidin-5-yl)pyridine-4-carboxamide
N-cyclohexyl-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-(2-oxopiperidin-4-yl)pyridine-4-carboxamide
2-{7-[(4-methanesulfonylpyridin-3-yl)amino]quinoxalin-5-yl}-4-methylbenzamide
8-(3-ethoxyphenyl)-N-(4-methanesulfonylpyridin-3-yl)quinoxalin-6-amine
N-(4-methanesulfonylpyridin-3-yl)-8-[3-(propan-2-yloxy)phenyl]quinoxalin-6-amine
8-(4-aminophenyl)-N-(4-methanesulfonylpyridin-3-yl)quinoxalin-6-amine
8-(3-aminophenyl)-N-(4-methanesulfonylpyridin-3-yl)quinoxalin-6-amine butyl 3-{[8-(1-methyl-1H-indol-6-yl)quinoxalin-6-yl]amino}pyridine-4-carboxylate
3{[8-(1-methyl-1H-indol-6-yl)quinoxalin-6-yl]amino}-N-[(morpholin-3-yl)methyl]pyridine-4-carboxamide
N-[(4-acetylmorpholin-3-yl)methyl]-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-[(4-methylmorpholin-2-yl)methyl]pyridine-4-carboxamide
N-[(1-acetylazetidin-3-yl)methyl]-3-{[8-(1-methyl-1H-indol-6-yl)quinoxalin-6-yl]amino}pyridine-4-carboxamide
N-[(4-acetylmorpholin-2-yl)methyl]-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-[(1-methylpyrrolidin-3-yl)methyl]pyridine-4-carboxamide
N-[(1-methyl-1H-imidazol-5-yl)methyl]-3-{[8-(1-methyl-1H-indol-6-yl)quinoxalin-6-yl]amino}pyridine-4-carboxamide
3-{[8-(1-methyl-1H-indol-6-yl)quinoxalin-6-yl]amino}-N-[(pyridazin-3-yl)methyl]pyridine-4-carboxamide
4-{[8-(1-methyl-1H-indol-6-yl)quinoxalin-6-yl]amino}pyridine-3-carbonitrile
N-(1-acetylpiperidin-4-yl)-3-{[8-(1-methyl-1H-indol-6-yl)quinoxalin-6-yl]amino}pyridine-4-carboxamide
N-(1-acetylpiperidin-3-yl)-3-{[8-(1-methyl-1H-indol-6-yl)quinoxalin-6-yl]amino}pyridine-4-carboxamide
5-{[8-(1-methyl-1H-indol-6-yl)quinoxalin-6-yl]amino}pyrimidine-4-carboxamide
3-{[8-(3-methyl-1-benzothiophen-5-yl)quinoxalin-6-yl]amino}pyridine-4-carbonitrile
3-{[8-(3-methyl-1-benzothiophen-5-yl)quinoxalin-6-yl]amino}-N-(1-methylpyrrolidin-3-yl)pyridine-4-carboxamide
N-(4-methanesulfonylpyridin-3-yl)-8-(4-methoxyphenyl)quinoxalin-6-amine
N-(4-methanesulfonylpyridin-3-yl)-8-(5-methoxy-2-methylphenyl)quinoxalin-6-amine
8-[1-(difluoromethyl)-1H-indol-6-yl]-N-(4-methanesulfonylpyridin-3-yl)quinoxalin-6-amine
8-(4-bromophenyl)-N-(4-methanesulfonylpyridin-3-yl)quinoxalin-6-amine
8-(3-bromophenyl)-N-(4-methanesulfonylpyridin-3-yl)quinoxalin-6-amine
2-aminopyrimidin-4-yl3-{[8-(1-methyl-1H-indol-6-yl)quinoxalin-6-yl]amino}pyridine-4-carboxylate
8-(1,2-benzothiazol-5-yl)-N-(4-methanesulfonylpyridin-3-yl)quinoxalin-6-amine
8-(2-amino-1,3-benzothiazol-5-yl)-N-(4-methanesulfonylpyridin-3-yl)quinoxalin-6-amine
N-(4-methanesulfonyl pyridin-3-yl)-8-[3-(trifluoromethoxy)phenyl]quinoxalin-6-amine
N-(4-{7-[(4-methanesulfonylpyridin-3-yl)amino]quinoxalin-5-yl}phenyl)pyrrolidine-2-carboxamide
N-(3-{7-[(4-methanesulfonylpyridin-3-yl)amino]quinoxalin-5-yl}phenyl)pyrrolidine-2-carboxamide
8-(1-ethyl-1H-indol-6-yl)-N-(4-methanesulfonylpyridin-3-yl)quinoxalin-6-amine
N-(4-methanesulfonylpyridin-3-yl)-8-(1-methyl-1H-1,2,3-benzotriazol-5-yl)quinoxalin-6-amine
2-{[8-(1-methyl-1H-indol-6-yl)quinoxalin-6-yl]amino}-N-[(1-methylpyrrolidin-3-yl)methyl]benzene-1-sulfonamide
N-(4-methanesulfonylpyridin-3-yl)-8-(2-methyl-1,3-benzothiazol-5-yl)quinoxalin-6-amine
N-(4-methanesulfonylpyridin-3-yl)-8-(1-methyl-1H-1,2,3-benzotriazol-6-yl)quinoxalin-6-amine
2-{[8-(1-methyl-1H-indol-6-yl)quinoxalin-6-yl]amino}-N-(1-methylpyrrolidin-3-yl)benzene-1-sulfonamide
2-{[8-(1-methyl-1H-indol-6-yl)quinoxalin-6-yl]amino}-N-[(oxan-4-yl)methyl]benzene-1-sulfonamide
2-{[8-(1-methyl-1H-indol-6-yl)quinoxalin-6-yl]amino}-N-[(1-methyl-1H-pyrazol-4-yl)methyl]benzene-1-sulfonamide
8-(2-amino-1,3-benzothiazol-6-yl)-N-(4-methanesulfonylpyridin-3-yl)quinoxalin-6-amine
N-{4-[(dimethylamino)methyl]pyridin-3-yl}-8-(1-methyl-1H-indol-6-yl)quinoxalin-6-amine
N-{2-[(dimethylamino)methyl]phenyl}-8-(1-methyl-1H-indol-6-yl)quinoxalin-6-amine
2-{[8-(1-methyl-1H-indol-6-yl)quinoxalin-6-yl]amino}benzoic acid
3-{[8-(1-methyl-1H-indol-6-yl)quinoxalin-6-yl]amino}pyridine-4-carboxylic acid
3-{[8-(1-methyl-1H-indol-6-yl)quinoxalin-6-yl]amino}-N-(1-methylazetidin-3-yl)pyridine-4-carboxamide
N-methyl-3-{[8-(1-methyl-1H-indol-6-yl)quinoxalin-6-yl]amino}-N-(1-methylpyrrolidin-3-yl)pyridine-4-carboxamide
2-{[8-(1-methyl-1H-indol-6-yl)quinoxalin-6-yl]amino}-N-(1-methylpyrrolidin-3-yl)benzamide
N-(5-{7-[(4-methanesulfonylpyridin-3-yl)amino]quinoxalin-5-yl}-1,3-benzothiazol-2-yl)pyrrolidine-2-carboxamide
N-(4-methanesulfonylpyridin-3-yl)-8-(1-propyl-1H-indol-6-yl)quinoxalin-6-amine
N-(6-{7-[(4-methanesulfonylpyridin-3-yl)amino]quinoxalin-5-yl}-1,3-benzothiazol-2-yl)pyrrolidine-2-carboxamide
N-(4-methanesulfonylpyridin-3-yl)-8-[4-(trifluoromethyl)phenyl]quinoxalin-6-amine
8-(4-amino-3-fluorophenyl)-N-(4-methanesulfonylpyridin-3-yl)quinoxalin-6-amine
N-methyl-2-{[8-(1-methyl-1H-indol-6-yl)quinoxalin-6-yl]amino}-N-[(pyrimidin-5-yl)methyl]benzene-1-sulfonamide
8-(4-fluorophenyl)-N-(4-methanesulfonylpyridin-3-yl)quinoxalin-6-amine
8-(1,4-dimethyl-1H-indol-6-yl)-N-(4-methanesulfonylpyridin-3-yl)quinoxalin-6-amine
8-(2-amino-1,3-benzothiazol-5-yl)-N-(2-methanesulfonylphenyl)quinoxalin-6-amine
N-(2-methanesulfonylphenyl)-8-(3-methyl-1-benzothiophen-5-yl)quinoxalin-6-amine
8-(3,5-diethylphenyl)-N-(4-methanesulfonylpyridin-3-yl)quinoxalin-6-amine
3-{[8-(3-methyl-1-benzothiophen-5-yl)quinoxalin-6-yl]amino}-N-[(3S)-1-methylpyrrolidin-3-yl]pyridine-4-carboxamide
3-{[8-(3-methyl-1-benzothiophen-5-yl)quinoxalin-6-yl]amino}-N-[(3R)-1-methylpyrrolidin-3-yl]pyridine-4-carboxamide
8-[2-(dimethylamino)-5-methylphenyl]-N-(4-methanesulfonylpyridin-3-yl)quinoxalin-6-amine
N-(1-methyl-1H-1,2,3-triazol-5-yl)-8-(1-methyl-1H-indol-6-yl)quinoxalin-6-amine
8-(1,5-dimethyl-1H-indol-6-yl)-N-(4-methanesulfonyl pyridin-3-yl)quinoxalin-6-amine
3-{[8-(4-fluoro-1-methyl-1H-indol-6-yl)quinoxalin-6-yl]amino}-N-(1-methylpyrrolidin-3-yl)pyridine-4-carboxamide
3-{[8-(1-methyl-1H-indol-6-yl)quinoxalin-6-yl]oxy}pyridine-4-carboxylic acid
2-{[8-(3-methyl-1-benzothiophen-5-yl)quinoxalin-6-yl]amino}-N-(1-methylpyrrolidin-3-yl)benzene-1-sulfonamide
N-(5-{7-[(4-methanesulfonylpyridin-3-yl)amino]quinoxalin-5-yl}-1-benzothiophen-2-yl)acetamide
8-[2-(dimethylamino)-1,3-benzothiazol-5-yl]-N-(4-methanesulfonylpyridin-3-yl)quinoxalin-6-amine
3-{[8-(1-methyl-1H-indol-6-yl)quinoxalin-6-yl]amino}-N-(pyrimidin-4-yl)pyridine-4-carboxamide
N-(1-acetylazetidin-3-yl)-3-{[8-(3-methyl-1-benzothiophen-5-yl)quinoxalin-6-yl]amino}pyridine-4-carboxamide
8-(1-methyl-1H-indol-6-yl)-N-(4-{[(pyrimidin-5-yl)amino]methyl}pyridin-3-yl)quinoxalin-6-amine
2-{[8-(1-methyl-1H-indol-6-yl)quinoxalin-6-yl]amino}-N-(1-methylpiperidin-3-yl)benzene-1-sulfonamide
2-{[8-(1-methyl-1H-indol-6-yl)quinoxalin-6-yl]amino}-N-(oxan-3-yl)benzene-1-sulfonamide
N-(4-methanesulfonylpyridin-3-yl)-8-[3-(methylsulfanyl)phenyl]quinoxalin-6-amine
N-(4-methanesulfonylpyridin-3-yl)-8-[3-(trifluoromethyl)-1-benzothiophen-5-yl]quinoxalin-6-amine
8-(4-bromo-3-fluorophenyl)-N-(4-methanesulfonylpyridin-3-yl)quinoxalin-6-amine
8-(4-bromo-2-methylphenyl)-N-(4-methanesulfonylpyridin-3-yl)quinoxalin-6-amine
N-(4-methanesulfonylpyridin-3-yl)-8-[4-(pentafluoro-λ⁶-sulfanyl)phenyl]-quinoxalin-6-amine
3-{[8-(2-amino-1,3-benzothiazol-5-yl)quinoxalin-6-yl]amino}-N-(1-methyl-pyrrolidin-3-yl)pyridine-4-carboxamide
3-{[8-(4-bromophenyl)quinoxalin-6-yl]amino}-N-(1-methylpyrrolidin-3-yl)-pyridine-4-carboxamide
N-(4-methanesulfonylpyridin-3-yl)-8-[2-(methylamino)-1,3-benzothiazol-5-yl]quinoxalin-6-amine
5-{7-[(4-methanesulfonylpyridin-3-yl)amino]quinoxalin-5-yl}-2,3-dihydro-1,3-benzothiazol-2-one (5-{7-[(4-methanesulfonylpyridin-3-yl)amino]quinoxalin-5-yl}-1,3-benzothiazol-2-ol)
8-(2-amino-1-benzothiophen-5-yl)-N-(4-methanesulfonylpyridin-3-yl)-quinoxalin-6-amine
8-(1-methyl-1H-indol-6-yl)-N-{4-[(methylamino)methyl]pyridin-3-yl}quinoxalin-6-amine
8-(3-methyl-1-benzothiophen-5-yl)-N-{4-[(methylamino)methyl]pyridin-3-yl}quinoxalin-6-amine
N-(5-bromopyrimidin-4-yl)-8-(1-methyl-1H-indol-6-yl)quinoxalin-6-amine
3-{[8-(3-methyl-1-benzothiophen-5-yl)quinoxalin-6-yl]amino}pyridine-4-carboxamide
3-{[8-(1-methyl-1H-indol-6-yl)quinoxalin-6-yl]amino}-N-(pyridin-3-yl)pyridine-4-carboxamide
8-(2-amino-1-benzothiophen-6-yl)-N-(4-methanesulfonylpyridin-3-yl)quinoxalin-6-amine
3-{[8-(1-methyl-1H-indol-6-yl)quinoxalin-6-yl]oxy}pyridine-4-carboxamide
3-{[8-(3-methyl-1-benzothiophen-5-yl)quinoxalin-6-yl]amino}-N-(5-oxo-pyrrolidin-3-yl)pyridine-4-carboxamide
3-{[8-(3-methyl-1-benzothiophen-5-yl)quinoxalin-6-yl]amino}-N-(2-oxo-pyrrolidin-3-yl)pyridine-4-carboxamide
3-{[8-(3-methyl-1-benzothiophen-5-yl)quinoxalin-6-yl]amino}-N-(1-methyl-5-oxopyrrolidin-3-yl)pyridine-4-carboxamide
3-{[8-(3-methyl-1-benzothiophen-5-yl)quinoxalin-6-yl]amino}-N-(1-methyl-2-oxopyrrolidin-3-yl)pyridine-4-carboxamide
8-(1-methyl-1H-indol-6-yl)-N-{4-[(methylamino)methyl]pyridin-3-yl}quinoxalin-6-amine
N-methyl-3-{[8-(3-methyl-1-benzothiophen-5-yl)quinoxalin-6-yl]amino}-pyridine-4-carboxamide
3-{[8-(4-bromophenyl)quinoxalin-6-yl]amino}-N-(1-methylpyrrolidin-3-yl)-pyridine-4-carboxamide
3-{[8-(2-amino-1,3-benzothiazol-5-yl)quinoxalin-6-yl]amino}-N-(1-methyl-pyrrolidin-3-yl)pyridine-4-carboxamide
N-(4-methanesulfonylpyridin-3-yl)-8-[4-(pentafluoro-λ⁶-sulfanyl)phenyl]quinoxalin-6-amine
3-{[8-(1-methyl-1H-indol-6-yl)quinoxalin-6-yl]amino}-N-(pyrimidin-4-yl)-pyridine-4-carboxamide
8-(1-methyl-1H-indol-6-yl)-N-(4-{[(pyrimidin-5-yl)amino]methyl}pyridin-3-yl)quinoxalin-6-amine
2-{[8-(1-methyl-1H-indol-6-yl)quinoxalin-6-yl]amino}-N-(1-methyl piperidin-3-yl)benzene-1-sulfonamide
2-{[8-(1-methyl-1H-indol-6-yl)quinoxalin-6-yl]amino}-N-(oxan-3-yl)benzene-1-sulfonamide
N-(4-methanesulfonylpyridin-3-yl)-8-[3-(methylsulfanyl)phenyl]quinoxalin-6-amine
8-(4-bromo-3-fluorophenyl)-N-(4-methanesulfonylpyridin-3-yl)quinoxalin-6-amine
8-(4-bromo-2-methylphenyl)-N-(4-methanesulfonylpyridin-3-yl)quinoxalin-6-amine
2-amino-N-(5-{7-[(4-methanesulfonylpyridin-3-yl)amino]quinoxalin-5-yl}-1-benzothiophen-2-yl)acetamide
N-(5-fluoro-1-methylpyrrolidin-3-yl)-3-{[8-(3-methyl-1-benzothiophen-5-yl)quinoxalin-6-yl]amino}pyridine-4-carboxamide
N-(3-fluoro-1-methylpyrrolidin-3-yl)-3-{[8-(3-methyl-1-benzothiophen-5-yl)quinoxalin-6-yl]amino}pyridine-4-carboxamide
N-(5,5-difluoro-1-methylpyrrolidin-3-yl)-3-{[8-(3-methyl-1-benzothiophen-5-yl)quinoxalin-6-yl]amino}pyridine-4-carboxamide
N-(3,3-difluoro-1-methylpyrrolidin-3-yl)-3-{[8-(3-methyl-1-benzothiophen-5-yl)quinoxalin-6-yl]amino}pyridine-4-carboxamide.

As used herein, the following definitions shall apply unless otherwise indicated or defined specifically elsewhere in the description and/or the claims for specific substituents, radicals, groups or moieties.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, such as one or more C═C double bond(s) and/or C≡C triple bond(s), but which is not aromatic (also referred to herein as “carbocycle”, “cycloaliphatic” or “cycloalkyl”), that has a single point of attachment to the rest of the molecule. Unless otherwise specified, aliphatic groups contain 1-8 or 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₇ hydrocarbon that is completely saturated or that contains one or more units of unsaturation, but which is not aromatic, that has a single point of attachment to the rest of the molecule. The term “alkyl” usually refers to a saturated aliphatic and acyclic moiety, while the term “alkenyl” usually refers to an unsaturated alphatic and acyclic moiety with one or more C═C double bonds and the term “alkynyl” usually refers to an aliphatic and acyclic moiety with one or more C≡C triple bonds. Exemplary aliphatic groups are linear or branched, substituted or unsubstituted C₁₋₈-alkyl, C₁₋₆-alkyl, C₂₋₈-alkenyl, C₂₋₆-alkenyl, C₂₋₈-alkynyl, C₂₋₆-alkynyl groups and hybrids thereof such as (cycloalkyl)alkyl, (cycloalkenyl)alkyl or (cycloalkyl)alkenyl.

In particular, the term “C₁₋₃-alkyl” refers to alkyl groups, i.e. saturated acyclic aliphatic groups, having 1, 2 or 3 carbon atoms. Exemplary C₁₋₃-alkyl groups are methyl, ethyl, propyl and isopropyl. The term “C₁₋₄-alkyl” refers to alkyl groups having 1, 2, 3 or 4 carbon atoms. Exemplary C₁₋₄-alkyl groups are methyl, ethyl, propyl, isopropyl, butyl, isobutyl, and tert-butyl. The term “C₁₋₆-alkyl” refers to alkyl groups having 1, 2, 3, 4, 5 or 6 carbon atoms. Exemplary C₁₋₆-alkyl groups are methyl, ethyl, propyl, isopropyl, butyl, isobutyl, tert-butyl, n-pentyl, 2-pentyl, n-hexyl, and 2-hexyl. The term “C₁₋₈-alkyl” refers to alkyl groups having 1, 2, 3, 4, 5, 6, 7, or 8 carbon atoms. Exemplary C₁₋₈-alkyl groups are methyl, ethyl, propyl, isopropyl, butyl, isobutyl, tert-butyl, n-pentyl, 2-pentyl, n-hexyl, 2-hexyl n-heptyl, 2-heptyl, n-octyl, 2-octyl, and 2,2,4-trimethylpentyl. Each of these alkyl groups may be straight-chain or except for C₁-alkyl and C₂-alkyl branched; they may be unsubstituted. However, in certain instances, which instances are usually specifically indicated in the definition of specific radicals, residues, moieties, groups or substituents elsewhere in this specification and/or the accompanying claims, each of these alkyl groups may be substituted with 1, 2 or 3 substituents that may be the same or different; typical examples of these substituents include but are not limited to halogen, hydroxy, alkoxy, unsubstituted or mono- or di-substituted amino.

In some instances, which instances are usually specifically indicated in the definition of specific radicals, residues, groups or substituents elsewhere in this specification and/or the accompanying claims, the C₁₋₃-alkyl, C₁₋₆-alkyl, C₁₋₈-alkyl groups may also comprise those residues in which 1 or 2 of non-terminal and non-adjacent —CH₂— (methylene) groups are replaced by —O—, —S— and/or 1 or 2 non-terminal and non-adjacent —CH₂— or —CH— groups are replaced by —NH— or —N—. These replacements yield, for instance, alkyl groups like —CH₂—CH₂—O—CH₃, —CH₂—CH₂—CH₂—S—CH₃, CH₂—CH₂—NH—CH₂—CH₃, CH₂—CH₂—O—CH₂—CH₂—O—CH₃, CH₂—CH₂—N(CH₃)—CH₂—CH₃, and the like. Further and/or different replacements of —CH— and —CH₂— groups may be defined for specific alkyl substituents or radicals elsewhere in the description and/or the claims.

The term “C₃₋₇-cycloalkyl” refers to a cycloaliphatic hydrocarbon, as defined above, with 3, 4, 5, 6 or 7 ring carbon atoms. C₃₋₇-cycloalkyl groups may be unsubstituted or substituted with—unless specified differently elsewhere in this specification 1, 2 or 3 substituents that may be the same of different and are unless specified differently elsewhere in this specification selected from the group comprising C₁₋₆-alkyl, O—C₁₋₆-alkyl (alkoxy), halogen, hydroxy unsubstituted or mono- or di-substituted amino. Exemplary C₃₋₇-cycloalkyl groups are cyclopropyl, 2-methyl-cyclopropyl, cyclopropenyl, cyclobutyl, cyclobutenyl, cyclopentyl, cyclopentenyl, cyclohexyl, cyclohexenyl, cycloheptyl, cycloheptenyl.

The term “alkoxy” refers to alkyl substituents and residues that are connected to another structural moiety via an oxygen atom (—O—). Sometimes, it is also referred to as “O-alkyl” and more specifically as “O—C₁₋₄-alkyl”, “O—C₁₋₆-alkyl”, “O—C₁₋₈-alkyl”. Like the similar alkyl groups, it may be straight-chain or except for —O—C₁-alkyl and —O—C₂-alkyl branched and may be unsubstituted or substituted with 1, 2 or 3 substituents that may be the same or different and are, if not specified differently elsewhere in this specification, selected from the group comprising halogen, unsubstituted or mono- or di-substituted amino. Exemplary alkoxy groups are methoxy, trifluoromethoxy, ethoxy, 2,2,2-trifluoroethoxy, n-propoxy, iso-propoxy, n-butoxy, sec-butoxy, tert-butoxy, n-pentoxy.

The term “alkylene” refers to a divalent alkyl group. An “alkylene chain” is a polymethylene group, i.e., —(CH₂)ₙ—, wherein n is a positive integer, preferably 1, 2, 3, 4, 5 or 6. In the context of the present invention “C₁₋₃-alkylene” refers to an alkylene moiety with 1, 2 and 3, respectively, —CH₂— groups; the term “alkylene”, however, not only comprises linear alkylene groups, i.e. “alkylene chains”, but branched alkylene groups as well. The term “C₁₋₆-alkylene” refers to an alkylene moiety that is either linear, i.e. an alkylene chain, or branched and has 1, 2, 3, 4, 5 or 6 carbon atoms. A substituted alkylene chain is a polymethylene group in which one or more methylene hydrogen atoms are replaced by (or with) a substituent. Suitable substituents include those described herein for a substituted alkyl group. In some instances 1 or 2 non-adjacent methylene groups of the alkylene chain may be replaced by, for instance, O, S and/or NH or N—C₁₋₄-alkyl. Exemplary alkylene groups are —CH₂—, —CH₂CH₂—, —CH₂CH₂CH₂CH₂—, —OCH₂—O—, —OCH₂CH₂—O—, —CH₂—NHCH₂CH₂—, —CH₂—N(CH₃)CH₂CH₂—.

The term “halogen” means F, Cl, Br, or I.

The term “heteroatom” means one or more of oxygen (O), sulfur (S), or nitrogen (N), including, any oxidized form of nitrogen or sulfur, e.g. N-oxides, sulfoxides and sulfones; the quaternized form of any basic nitrogen or a substitutable nitrogen of a heterocyclic or heteroaromatic ring, for example N (as in 3,4-dihydro-2H-pyrrolyl), NH (as in pyrrolidinyl) or N-SUB with SUB being a suitable substituent (as in N-substituted pyrrolidinyl).

The term “aryl” used alone or as part of a larger moiety as in “aralkyl”, “aralkoxy”, or “aryloxyalkyl”, refers to monocyclic, bicyclic and tricyclic ring systems having a total of five to fourteen ring members, that ring members being carbon atoms, wherein at least one ring in the system is aromatic, i.e., it has (4n+2)π (pi) electrons (with n being an integer selected from 0, 1, 2, 3), which electrons are delocalized over the system, and wherein each ring in the system contains three to seven ring members. Preferably, all rings in the aryl system or the entire ring system are aromatic. The term “aryl” is used interchangeably with the term “aryl ring”. In certain embodiments of the present invention, “aryl” refers to an “aromatic ring system”. More specifically, those aromatic ring systems may be mono-, bi- or tricyclic with 5, 6, 7, 8, 9, 10, 11, 12, 13, 14 ring carbon atoms. Even more specifically, those aromatic ring systems may be mono- or bicyclic with 6, 7, 8, 9, 10 ring carbon atoms.

Exemplary aryl groups are phenyl, biphenyl, naphthyl, anthracyl and the like, which may be unsubstituted or substituted with one or more identical or different substituents. Also included within the scope of the terms “aryl” or “aromatic ring system”, as they are used herein, is a group in which an aromatic ring is fused to one or more nonaromatic rings, such as indanyl, phthalimidyl, naphthimidyl, phenanthridinyl, or tetrahydronaphthyl, and the like. In the latter case the “aryl” group or substituent is attached to its pendant group via the aromatic part of the ring system.

The terms “heteroaryl” and “heteroar-”, used alone or as part of a larger moiety, e.g., “heteroaralkyl”, or “heteroaralkoxy”, refer to groups having 3, 4, 5, 6, 7, 8, 9, 10, 11, 12, 13, 14 ring atoms (which atoms are carbon and hetero atoms), preferably 5, 6, or 9 ring atoms; having 6, 10, or 14 π (pi) electrons shared in a cyclic array; and having, in addition to carbon atoms, 1, 2, 3, 4 or 5 heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, furazanyl, pyridyl, pyridazinyl, pyrimidinyl, pyrazinyl, indolizinyl, purinyl, naphthyridinyl, pteridinyl, and pyrrolopyridinyl, in particular pyrrolo[2,3-b]pyridinyl. The terms “heteroaryl” and “heteroar-”, as used herein, also include groups in which a heteroaromatic ring is fused to one or more aryl, cycloaliphatic, or heterocyclyl rings, where the radical or point of attachment is preferably on the heteroaromatic or, if present, the aryl ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. For example, an indolyl ring may be attached via one of the ring atoms of the six-membered aryl ring or via one of the ring atoms of the five-membered heteroaryl ring. A heteroaryl group is optionally mono-, bi- or tricyclic. The term “heteroaryl” is used interchangeably with the terms “heteroaryl ring”, “heteroaryl group”, or “heteroaromatic”, any of which terms include rings that are unsubstituted or substituted with one or more identical or different substituents. The term “heteroaralkyl” refers to an alkyl group substituted by a heteroaryl, wherein the alkyl and heteroaryl portions independently are optionally substituted.

A heteroaryl ring can be attached to its pendant group at any of its hetero or carbon ring atoms which attachment results in a stable structure or molecule: any of the ring atoms may be unsubstituted or substituted.

The structures of typical examples of “heteroaryl” substituents as used in the present invention are depicted below:

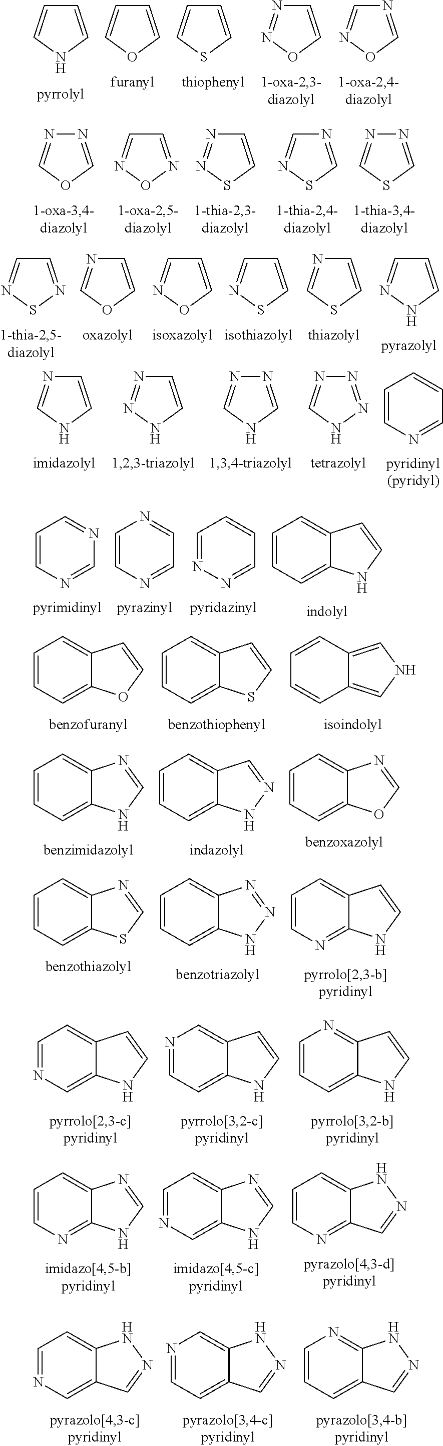

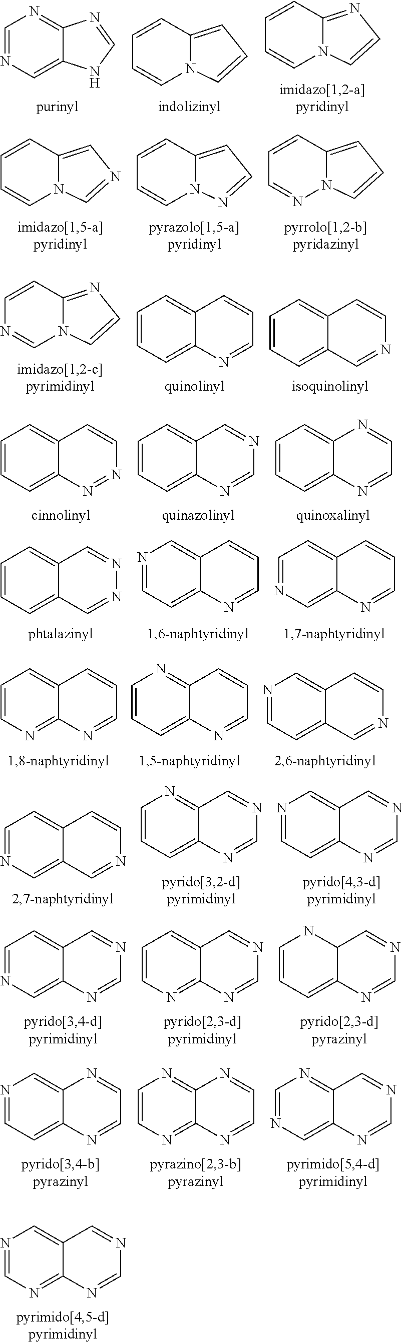

Those heteroaryl substituents can be attached to any pendant group via any of its ring atoms suitable for such an attachment.

As used herein, the terms “heterocycle”, “heterocyclyl”, “heterocyclic radical”, and “heterocyclic ring” are used interchangeably and refer to a stable mono-bi- or tricyclic heterocyclic moiety with 5, 6, 7, 8, 9, 10, 11, 12, 13, 14 ring atoms wherein 1, 2, 3, 4, 5 of said ring atoms are hetero atoms and wherein that heterocyclic moiety is either saturated or partially unsaturated. Preferably, the heterocycle is a stable saturated or partially unsaturated 3-, 4-, 5-, 6-, or 7-membered monocyclic or 7-, 8-, 9-, 10-, or 11-membered bicyclic or 11-, 12-, 13-, or 14-membered tricyclic heterocyclic moiety.

When used in reference to a ring atom of a heterocycle, the term “nitrogen” includes a substituted nitrogen. As an example, in a saturated or partially unsaturated ring having 1-3 heteroatoms selected from oxygen, sulfur or nitrogen, the nitrogen is N (as in 3,4-dihydro-2H-pyrrolyl), NH (as in pyrrolidinyl), or N-SUB with SUB being a suitable substituent (as in N substituted pyrrolidinyl).

In the context of the term “heterocycle” the term “saturated” refers to a completely saturated heterocyclic system, like pyrrolidinyl, piperidinyl, morpholinyl, and piperidinonyl. With regard to the term “heterocycle” the term “partially unsaturated” refers to heterocyclic systems (i) that contain one or more units of unsaturation, e.g. a C═C or a C=Heteroatom bond, but that are not aromatic, for instance, tetrahydropyridinyl; or (ii) in which a (saturated or unsaturated but non-aromatic) heterocyclic ring is fused with an aromatic or heteroaromatic ring system, wherein, however, the “partially unsaturated heterocycle” is attached to the rest of the molecule (its pendant group) via one of the ring atoms of the “heterocyclic” part of the system and not via the aromatic or heteroaromatic part. This first class (i) of “partially unsaturated” heterocycles may also be referred to as “non-aromatic partially unsaturated” heterocycles. This second class (ii) of “partially unsaturated” heterocycles may also be referred to as (bicyclic or tricyclic) “partially aromatic” heterocycles indicating that at least one of the rings of that heterocycle is a saturated or unsaturated but non-aromatic heterocycle that is fused with at least one aromatic or heteroaromatic ring system. Typical examples of these “partially aromatic” heterocycles are 1,2,3,4-tetrahydroquinolinyl and 1,2,3,4-tetrahydroisoquinolinyl.

A heterocyclic ring can be attached to its pendant group at any heteroatom or carbon atom that results in a stable structure and any of the ring atoms may be unsubstituted or substituted. Examples of such saturated or partially unsaturated heterocyclic radicals include, without limitation, tetrahydrofuranyl, tetrahydropyranyl, tetrahydrothiophenyl pyrrolidinyl, piperidinyl, pyrrolinyl, morpholinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group is optionally mono, bi- or tricyclic. The term “heterocyclylalkyl” refers to an alkyl group substituted by a heterocyclyl, wherein the alkyl and heterocyclyl portions independently are unsubstituted or substituted.

The term “unsaturated”, as used herein, means that a moiety has one or more units of unsaturation.

As used herein with reference to any rings, ring systems, ring moieties, and the like, the term “partially unsaturated” refers to a ring moiety that includes at least one double or triple bond. The term “partially unsaturated” is intended to encompass rings having multiple sites of unsaturation. In particular, it encompasses (i) non-saturated (mono-, bi- or tricyclic) ring systems without any aromatic or heteroaromatic moiety or part; and (ii) bi- or tricyclic ring systems in which one of the rings of that system is an aromatic or heteroaromatic ring which is fused with another ring that is neither an aromatic nor a heteroaromatic ring, e.g. tetrahydronaphthyl or tetrahydroquinolinyl. The first class (i) of “partially unsaturated” rings, ring systems, ring moieties may also be referred to as “non-aromatic partially unsaturated” rings, ring systems, ring moieties, while the second class (ii) may be referred to as “partially aromatic” rings, ring systems, ring moieties.

As described herein, certain compounds of the invention contain “substituted” or “optionally substituted” moieties. In general, the term “substituted”, whether preceded by the term “optionally” or not, means that one or more hydrogens of the designated moiety are replaced with a suitable substituent. “Substituted” applies to one or more hydrogens that are either explicit or implicit from the structure. Unless otherwise indicated, a “substituted” or “optionally substituted” group has a suitable substituent at each substitutable position of the group, and when more than one position in any given structure is substituted with more than one substituent selected from a specified group, the substituent is either the same or different at every position. If a certain group, substituent, moiety or radical is “mono-substituted”, it bears one (1) substituent. If it is “di-substituted”, it bears two (2) substituents, being either the same or different; if it is “tri-substituted”, it bears three (3) substituents, wherein all three are the same or two are the same and the third is different or all three are different from each other. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

In the context of the present invention the term “derivative” means any non-toxic salt, ester, salt of an ester or other derivative of a compound of this invention that, upon administration to a recipient, is capable of providing, either directly or indirectly, a compound of this invention or an inhibitory active metabolite or residue thereof.

The compounds of the present invention can be in the form of a prodrug compound. “Prodrugs” and “prodrug compound” mean a derivative that is converted into a biologically active compound according to the present invention under physiological conditions in the living body, e.g., by oxidation, reduction, hydrolysis or the like, each of which is carried out enzymatically, or without enzyme involvement. Examples of prodrugs are compounds, in which the amino group in a compound of the present invention is acylated, alkylated or phosphorylated, e.g., eicosanoylamino, alanylamino, pivaloyloxymethylamino or in which the hydroxyl group is acylated, alkylated, phosphorylated or converted into the borate, e.g. acetyloxy, palmitoyloxy, pivaloyloxy, succinyloxy, fumaryloxy, alanyloxy or in which the carboxyl group is esterified or amidated, or in which a sulfhydryl group forms a disulfide bridge with a carrier molecule, e.g. a peptide, that delivers the drug selectively to a target and/or to the cytosol of a cell. These compounds can be produced from compounds of the present invention according to well-known methods. Other examples of prodrugs are compounds, wherein the carboxylate in a compound of the present invention is for example converted into an alkyl-, aryl-, choline-, amino-, acyloxymethylester, linolenoyl-ester.

The term “solvates” means addition forms of the compounds of the present invention with solvents, preferably pharmaceutically acceptable solvents, that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, e.g. a mono- or dihydrate. If the solvent is alcohol, the solvate formed is an alcoholate, e.g., a methanolate or ethanolate. If the solvent is an ether, the solvate formed is an etherate, e.g., diethyl etherate.

The term “N-oxides” means such compounds of the present invention that contain an amine oxide moiety, i.e. the oxide of a tertiary amine group.

The compounds of formula (I) may have one or more centres of chirality. They may accordingly occur in various enantiomeric and diastereomeric forms, as the case may be, and be in racemic or optically active form. The invention, therefore, also relates to the optically active forms, enantiomers, racemates, diastereomers, mixtures thereof in all ratios, collectively: “stereoisomers” for the purpose of the present invention, of these compounds. Since the pharmaceutical activity of the racemates or stereo-isomers of the compounds according to the invention may differ, it may be desirable to use a specific stereoisomer, e.g. one specific enantiomer or diastereomer. In these cases, a compound according to the present invention obtained as a racemate—or even intermediates thereof—may be separated into the stereoisomeric (enantiomeric, diastereoisomeric) compounds by chemical or physical measures known to the person skilled in the art. Another approach that may be applied to obtain one or more specific stereoisomers of a compound of the present invention in an enriched or pure form makes use of stereoselective synthetic procedures, e.g. applying starting material in a stereoisomerically enriched or pure form (for instance using the pure or enriched (R)- or (S)-enantiomer of a particular starting material bearing a chiral center) or utilizing chiral reagents or catalysts, in particular enzymes. In the context of the present invention the term “pure enantiomer” usually refers to a relative purity of one enantiomer over the other (its antipode) of equal to or greater than 95%, preferably ≥98%, more preferably ≥98.5%, still more preferably 99%.

Thus, for example, the compounds of the invention which have one or more centers of chirality and which occur as racemates or as mixtures of enantiomers or diastereoisomers can be fractionated or resolved by methods known per se into their optically pure or enriched isomers, i.e. enantiomers or diastereomers. The separation of the compounds of the invention can take place by chromatographic methods, e.g. column separation on chiral or nonchiral phases, or by recrystallization from an optionally optically active solvent or by use of an optically active acid or base or by derivatization with an optically active reagent such as, for example, an optically active alcohol, and subsequent elimination of the radical.

In the context of the present invention the term “tautomer” refers to compounds of the present invention that may exist in tautomeric forms and show tautomerism; for instance, carbonyl compounds may be present in their keto and/or their enol form and show keto-enol tautomerism. Those tautomers may occur in their individual forms, e.g., the keto or the enol form, or as mixtures thereof and are claimed separately and together as mixtures in any ratio. The same applies for cis/trans isomers, E/Z isomers, conformers and the like.

The compounds of the present invention can be in the form of a pharmaceutically acceptable salt, a pharmaceutically acceptable solvate, or a pharmaceutically acceptable solvate of a pharmaceutically acceptable salt.

The term “pharmaceutically acceptable salts” refers to salts prepared from pharmaceutically acceptable bases or acids, including inorganic bases or acids and organic bases or acids. In cases where the compounds of the present invention contain one or more acidic or basic groups, the invention also comprises their corresponding pharmaceutically acceptable salts. Thus, the compounds of the present invention which contain acidic groups can be present in salt form, and can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. Compounds of the present invention which contain one or more basic groups, e.g. groups which can be protonated, can be present in salt form, and can be used according to the invention in the form of their addition salts with inorganic or organic acids. Examples of suitable acids include hydrogen chloride, hydrogen bromide, hydrogen iodide, phosphoric acid, sulfuric acid, nitric acid, methanesulfonic acid, p-toluenesulfonic acid, naphthalenedisulfonic acid, sulfoacetic acid, trifluoroacetic acid, oxalic acid, acetic acid, tartaric acid, lactic acid, salicylic acid, benzoic acid, carbonic acid, formic acid, propionic acid, pivalic acid, diethylacetic acid, malonic acid, succinic acid, pimelic acid, fumaric acid, malonic acid, maleic acid, malic acid, embonic acid, mandelic acid, sulfaminic acid, phenylpropionic acid, gluconic acid, ascorbic acid, isonicotinic acid, citric acid, adipic acid, taurocholic acid, glutaric acid, stearic acid, glutamic acid or aspartic acid, and other acids known to the person skilled in the art. The salts which are formed are, inter alia, hydrochlorides, chlorides, hydrobromides, bromides, iodides, sulfates, phosphates, methanesulfonates (mesylates), tosylates, carbonates, bicarbonates, formates, acetates, sulfoacetates, triflates, oxalates, malonates, maleates, succinates, tartrates, malates, embonates, mandelates, fumarates, lactates, citrates, glutarates, stearates, aspartates and glutamates. The stoichiometry of the salts formed from the compounds of the invention may moreover be an integral or non-integral multiple of one.

If the compounds of the present invention simultaneously contain acidic and basic groups in the molecule, the invention also includes, in addition to the salt forms mentioned, inner salts or betaines (zwitterions). The respective salts can be obtained by customary methods which are known to a person skilled in the art, for example by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Therefore, the following items are also in accordance with the invention:

(a) all stereoisomers or tautomers of the compounds, including mixtures thereof in all ratios;
(b) prodrugs of the compounds, or stereoisomers or tautomers of these prodrugs;
(c) pharmaceutically acceptable salts of the compounds and of the items mentioned under (a) and (b);
(d) pharmaceutically acceptable solvates of the compounds and of the items mentioned under (a), (b) and (c);
(e) N-oxides of the compounds and of the items mentioned under (a), (b), (c), and (d).

It should be understood that all references to compounds above and below are meant to include these items, in particular pharmaceutically acceptable solvates of the compounds, or pharmaceutically acceptable solvates of their pharmaceutically acceptable salts.

Furthermore, the present invention relates to pharmaceutical compositions comprising at least one compound of formula (I), or its derivatives, prodrugs, solvates, tautomers or stereoisomers thereof as well as the physiologically acceptable salts of each of the foregoing, including mixtures thereof in all ratios, as active ingredient, together with a pharmaceutically acceptable carrier.

For the purpose of the present invention the term “pharmaceutical composition” refers to a composition or product comprising one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing at least one compound of the present invention and a pharmaceutically acceptable carrier. It may further comprise physiologically acceptable excipients, auxiliaries, adjuvants, diluents and/or additional pharmaceutically active substance other than the compounds of the invention.

The pharmaceutical compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

A pharmaceutical composition of the present invention may additionally comprise one or more other compounds as active ingredients (drugs), such as one or more additional compounds of the present invention. In a particular embodiment the pharmaceutical composition further comprises a second active ingredient or its derivatives, prodrugs, solvates, tautomers or stereoisomers thereof as well as the physiologically acceptable salts of each of the foregoing, including mixtures thereof in all ratios, wherein that second active ingredient is other than a compound of formula (I); preferably, that second active ingredient is a compound that is useful in the treatment, prevention, suppression and/or amelioration of medicinal conditions or pathologies for which the compounds of the present invention are useful as well and which are listed elsewhere hereinbefore or hereinafter. Such combination of two or more active ingredients or drugs may be safer or more effective than either drug or active ingredient alone, or the combination is safer or more effective than it would be expected based on the additive properties of the individual drugs. Such other drug(s) may be administered, by a route and in an amount commonly used contemporaneously or sequentially with a compound of the invention. When a compound of the invention is used contemporaneously with one or more other drugs or active ingredients, a combination product containing such other drug(s) and the compound of the invention—also referred to as “fixed dose combination”—is preferred. However, combination therapy also includes therapies in which the compound of the present invention and one or more other drugs are administered on different overlapping schedules. It is contemplated that when used in combination with other active ingredients, the compound of the present invention or the other active ingredient or both may be used effectively in lower doses than when each is used alone. Accordingly, the pharmaceutical compositions of the present invention include those that contain one or more other active ingredients, in addition to a compound of the invention.

The compounds of the present invention can be used as medicaments. They exhibit pharmacological activity by inhibiting 6-phosphofructo-2-kinase/fructose-2,6-bisphosphatase (PFKFB), in particular its isoforms PFKFB3 and/or PFKFB4, more particular PFKFB3. Even more particular, the compounds of the present invention exhibit inhibition of the kinase enzymatic activity of PFKFB, especially of PFKFB3 and/or PFKFB4, more especially of PFKFB3. Thus, they are useful for the treatment, prevention, suppression and/or amelioration of medicinal conditions or pathologies that are affected by PFKFB activity, in particular by PFKFB3 and/or PFKFB4 activity, more particular by PFKFB3 activity. The compounds of the present invention are thus particularly useful for the treatment of a hyperproliferative disorder. More specifically, they are useful for the treatment of a disorder or disease selected from the group consisting of cancer, in particular adipose cancer, anogenital cancer, astrocytoma cancer, bladder cancer, breast cancer, central nervous system cancer, cervical cancer, colon cancer, connective tissue cancer, glioblastoma, glioma, kidney cancer, leukemia, lung cancer, lymphoid cancer, ovarian cancer, pancreatic cancer, prostate cancer, retinal cancer, skin cancer, stomach cancer, thyroid cancer, uterine cancer.

Furthermore, some of the compounds of formula (I) may not only exhibit inhibiting activity on PFKFB but further exhibit activity by modulating the activity of other pharmacological target molecules than PFKFB, for instance autotaxin, Brk, BTK, cyclophilin, ERK, Gcn2, hexokinase I, hexokinase II, IKK-epsilon, IRAK1, IRAK4, Ire1, JNK, LDHA/B, LPA, PDK-1, TGF-beta or VEGF target molecules which modulating activity may be useful for the treatment of one or more of the hyperproliferative disorders mentioned above. Thus, those compounds of formula (I) exhibiting activity on PFKFB and another pharmacological target may also be described as having a dual mode of action and may allow for targeting two different target molecules involved in the genesis and progression of a hyperproliferative disorder, in particular cancer.

The disclosed compounds of the formula (I) can be administered and/or used in combination with other known therapeutic agents, including anticancer agents. As used herein, the term “anticancer agent” relates to any agent which is administered to a patient with cancer for the purposes of treating the cancer.

The anti-cancer treatment defined above may be applied as a monotherapy or may involve, in addition to the herein disclosed compounds of formula (I), conventional surgery or radiotherapy or medicinal therapy. Such medicinal therapy, e.g. a chemotherapy or a targeted therapy, may include one or more, but preferably one, of the following anti-tumor agents:

alkylating Agents
such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone;
apaziquone, fotemustine, glufosfamide, palifosfamide, pipobroman, trofosfamide, uramustine, TH-302⁴, VAL-083⁴;

### Platinum Compounds

such as carboplatin, cisplatin, eptaplatin, miriplatine hydrate, oxaliplatin, lobaplatin, nedaplatin, picoplatin, satraplatin;

### DNA Altering Agents

such as amrubicin, bisantrene, decitabine, mitoxantrone, procarbazine, trabectedin, clofarabine;
amsacrine, brostallicin, pixantrone, laromustine^{1,3};

### Topoisomerase Inhibitors

such as etoposide, irinotecan, razoxane, sobuzoxane, teniposide, topotecan; amonafide, belotecan, elliptinium acetate, voreloxin;

### Microtubule Modifiers

such as cabazitaxel, docetaxel, eribulin, ixabepilone, paclitaxel, vinblastine, vincristine, vinorelbine, vindesine, vinflunine;
fosbretabulin, tesetaxel;

### Antimetabolites

such as asparaginase³, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur;
doxifluridine, elacytarabine, raltitrexed, sapacitabine, tegafur^{2,3}, trimetrexate;

### Anticancer Antibiotics

such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin;
aclarubicin, peplomycin, pirarubicin;

### Hormones/Antagonists

such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol;
acolbifene, danazol, deslorelin, epitiostanol, orteronel, enzalutamide^{1,3};

### Aromatase Inhibitors

such as aminoglutethimide, anastrozole, exemestane, fadrozole, letrozole, testolactone;
formestane;

### Small Molecule Kinase Inhibitors

such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib;
afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib⁴, cabozantinib S-malate^{1,3}, ibrutinib^{1,3}, icotinib⁴, buparlisib², cipatinib⁴, cobimetinib^{1,3}, fedratinib¹, XL-647⁴;

### Photosensitizers

such as methoxsalen³;
porfimer sodium, talaporfin, temoporfin;

### Antibodies

such as alemtuzumab, besilesomab, brentuximab vedotin, cetuximab, denosumab, ipilimumab, ofatumumab, panitumumab, rituximab, tositumomab,
trastuzumab, bevacizumab, pertuzumab^{2,3};
catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab^{1,2,3}, onartuzumab^{1,3}, racotumomab¹, tabalumab^{1,3}, EMD-525797⁴, nivolumab^{1,3};

### Cytokines

such as aldesleukin, interferon alfa², interferon alfa2a³, interferon alfa2b^{2,3}; celmoleukin, tasonermin, teceleukin, oprelvekin^{1,3}, recombinant interferon beta-1a⁴;

### Drug Conjugates

such as denileukin diftitox, ibritumomab tiuxetan, iobenguane I123, prednimustine, trastuzumab emtansine, estramustine, gemtuzumab, ozogamicin, aflibercept;
cintredekin besudotox, edotreotide, inotuzumab ozogamicin, naptumomab estafenatox, oportuzumab monatox, technetium (99mTc) arcitumomab^{1,3}, vintafolide^{1,3};

### Vaccines

such as sipuleucel³; vitespen³, emepepimut-S³, oncoVAX⁴, rindopepimut³, troVax⁴, MGN-1601⁴, MGN-1703⁴;

### Miscellaneous

alitretinoin, bexarotene, bortezomib, everolimus, ibandronic acid, imiquimod, lenalidomide, lentinan, metirosine, mifamurtide, pamidronic acid, pegaspargase, pentostatin, sipuleuceI³, sizofiran, tamibarotene, temsirolimus, thalidomide, tretinoin, vismodegib, zoledronic acid, vorinostat; celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine⁴,
picibanil⁴, reolysin⁴, retaspimycin hydrochloride^{1,3}, trebananib^{2,3}, virulizin⁴, carfilzomib^{1,3}, endostatin⁴, immucothel⁴, belinostat³, MGN-1703⁴;
¹Prop. INN (Proposed International Nonproprietary Name)
²Rec. INN (Recommended International Nonproprietary Names)

### ³USAN (United States Adopted Name)

### ⁴no INN.

A further embodiment of the present invention is a process for the manufacture of the pharmaceutical compositions of the present invention, characterized in that one or more compounds according to the invention and one or more compounds selected from the group consisting of solid, liquid or semiliquid excipients, auxiliaries, adjuvants, diluents, carriers and pharmaceutically active agents other than the compounds according to the invention, are converted in a suitable dosage form.

In another aspect of the invention, a set or kit is provided comprising a therapeutically effective amount of at least one compound of the invention and/or at least one pharmaceutical composition as described herein and a therapeutically effective amount of at least one further pharmacologically active substance other than the compounds of the invention. It is preferred that this set or kit comprises separate packs of

a) an effective amount of a compound of formula (I), or its derivatives, prodrugs, solvates, tautomers or stereoisomers thereof as well as the physiologically acceptable salts of each of the foregoing, including mixtures thereof in all ratios, and
b) an effective amount of a further active ingredient that further active ingredient not being a compound of formula (I).

The pharmaceutical compositions of the present invention may be administered by any means that achieve their intended purpose. For example, administration may be via oral, parenteral, topical, enteral, intravenous, intramuscular, inhalant, nasal, intraarticular, intraspinal, transtracheal, transocular, subcutaneous, intraperitoneal, transdermal, or buccal routes. Alternatively, or concurrently, administration may be via the oral route. The dosage administered will be dependent upon the age, health, and weight of the recipient, kind of concurrent treatment, if any, frequency of treatment, and the nature of the effect desired. Parenteral administration is preferred. Oral administration is especially preferred.

Suitable dosage forms include, but are not limited to capsules, tablets, pellets, dragees, semi-solids, powders, granules, suppositories, ointments, creams, lotions, inhalants, injections, cataplasms, gels, tapes, eye drops, solution, syrups, aerosols, suspension, emulsion, which can be produced according to methods known in the art, for example as described below:

Tablets: mixing of active ingredient/s and auxiliaries, compression of said mixture into tablets (direct compression), optionally granulation of part of mixture before compression.
Capsules: mixing of active ingredient/s and auxiliaries to obtain a flowable powder, optionally granulating powder, filling powders/granulate into opened capsules, capping of capsules.
Semi-solids (ointments, gels, creams): dissolving/dispersing active ingredient/s in an aqueous or fatty carrier; subsequent mixing of aqueous/fatty phase with complementary fatty/aqueous phase, homogenization (creams only).
Suppositories (rectal and vaginal): dissolving/dispersing active ingredient/s in carrier material liquified by heat (rectal: carrier material normally a wax; vaginal: carrier normally a heated solution of a gelling agent), casting said mixture into suppository forms, annealing and withdrawal suppositories from the forms.
Aerosols: dispersing/dissolving active agent/s in a propellant, bottling said mixture into an atomizer.

In general, non-chemical routes for the production of pharmaceutical compositions and/or pharmaceutical preparations comprise processing steps on suitable mechanical means known in the art that transfer one or more compounds of the invention into a dosage form suitable for administration to a patient in need of such a treatment. Usually, the transfer of one or more compounds of the invention into such a dosage form comprises the addition of one or more compounds, selected from the group consisting of carriers, excipients, auxiliaries and pharmaceutical active ingredients other than the compounds of the invention. Suitable processing steps include, but are not limited to combining, milling, mixing, granulating, dissolving, dispersing, homogenizing, casting and/or compressing the respective active and nonactive ingredients. Mechanical means for performing said processing steps are known in the art, for example from Ullmann's Encyclopedia of Industrial Chemistry, 5th Edition. In this respect, active ingredients are preferably at least one compound of the invention and optionally one or more additional compounds other than the compounds of the invention, which show valuable pharmaceutical properties, preferably those pharmaceutical active agents other than the compounds of the invention, which are disclosed herein.

Particularly suitable for oral use are tablets, pills, coated tablets, capsules, powders, granules, syrups, juices or drops, suitable for rectal use are suppositories, suitable for parenteral use are solutions, preferably oil-based or aqueous solutions, furthermore suspensions, emulsions or implants, and suitable for topical use are ointments, creams or powders. The compounds of the invention may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, dyes, flavours and/or a plurality of further active ingredients, for example one or more vitamins.

Suitable excipients are organic or inorganic substances, which are suitable for enteral (for example oral), parenteral or topical administration and do not react with the compounds of the invention, for example water, vegetable oils, benzyl alcohols, alkylene glycols, polyethylene glycols, glycerol triacetate, gelatine, carbohydrates, such as lactose, sucrose, mannitol, sorbitol or starch (maize starch, wheat starch, rice starch, potato starch), cellulose preparations and/or calcium phosphates, for example tricalcium phosphate or calcium hydrogen phosphate, magnesium stearate, talc, gelatine, tragacanth, methyl cellulose, hydroxypropylmethylcellulose, sodium carboxymethylcellulose, polyvinyl pyrrolidone and/or vaseline.

If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries include, without limitation, flow-regulating agents and lubricants, for example, silica, talc, stearic acid or salts thereof, such as magnesium stearate or calcium stearate, and/or polyethylene glycol. Dragee cores are provided with suitable coatings, which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices or to provide a dosage form affording the advantage of prolonged action, the tablet, dragee or pill can comprise an inner dosage and an outer dosage component the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol, solutions of suitable cellulose preparations such as acetyl-cellulose phthalate, cellulose acetate or hydroxypropylmethyl-cellulose phthalate, are used. Dye stuffs or pigments may be added to the tablets or dragee coatings, for example, for identification or in order to characterize combinations of active compound doses.

Suitable carrier substances are organic or inorganic substances which are suitable for enteral (e.g. oral) or parenteral administration or topical application and do not react with the novel compounds, for example water, vegetable oils, benzyl alcohols, polyethylene glycols, gelatin, carbohydrates such as lactose or starch, magnesium stearate, talc and petroleum jelly. In particular, tablets, coated tablets, capsules, syrups, suspensions, drops or suppositories are used for enteral administration, solutions, preferably oily or aqueous solutions, furthermore suspensions, emulsions or implants, are used for parenteral administration, and ointments, creams or powders are used for topical application. The compounds of the invention can also be lyophilized and the lyophilizates obtained can be used, for example, for the production of injection preparations.

Other pharmaceutical preparations, which can be used orally include push-fit capsules made of gelatine, as well as soft, sealed capsules made of gelatine and a plasticizer such as glycerol or sorbitol. The push-fit capsules can contain the active compounds in the form of granules, which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

The liquid forms in which the novel compositions of the present invention may be incorporated for administration orally include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatine.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400).

Aqueous injection suspensions may contain substances, which increase the viscosity of the suspension, including, for example, sodium carboxymethyl cellulose, sorbitol, and/or dextran, optionally, the suspension may also contain stabilizers.

For administration as an inhalation spray, it is possible to use sprays in which the active ingredient is either dissolved or suspended in a propellant gas or propellant gas mixture (for example CO₂ or chlorofluorocarbons). The active Ingredient is advantageously used here in micronized form, in which case one or more additional physiologically acceptable solvents may be present, for example ethanol. Inhalation solutions can be administered with the aid of conventional inhalers.

Possible pharmaceutical preparations, which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatine rectal capsules, which consist of a combination of the active compounds with a base. Possible base materials include, for example, liquid triglycerides, polyethylene glycols, or paraffin hydrocarbons.

For use in medicine, the compounds of the present invention may be in the form of pharmaceutically acceptable salts. Other salts may, however, be useful in the preparation of the compounds of the invention or of their pharmaceutically acceptable salts. Suitable pharmaceutically acceptable salts of the compounds of this invention are those described hereinbefore and include acid addition salts which may, for example be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic bases, e.g. quaternary ammonium salts.

The pharmaceutical preparations can be employed as medicaments in human and veterinary medicine. As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. Said therapeutic effective amount of one or more of the compounds of the invention is known to the skilled artisan or can be easily determined by standard methods known in the art.

The compounds of the present invention and the optional additional active substances are generally administered analogously to commercial preparations. Usually, suitable doses that are therapeutically effective lie in the range between 0.0005 mg and 1000 mg, preferably between 0.005 mg and 500 mg and especially between 0.5 mg and 100 mg per dose unit. The daily dose is preferably between about 0.001 mg/kg and 10 mg/kg of body weight.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

The specific dose for the individual patient, in particular for the individual human patient, depends, however, on the multitude of factors, for example on the efficacy of the specific compounds employed, on the age, body weight, general state of health, the sex, the kind of diet, on the time and route of administration, on the excretion rate, the kind of administration and the dosage form to be administered, the pharmaceutical combination and severity of the particular disorder to which the therapy relates. The specific therapeutic effective dose for the individual patient can readily be determined by routine experimentation, for example by the doctor or physician, which advises or attends the therapeutic treatment.

The compounds of the present invention can be prepared according to the procedures of the following Schemes and Examples, using appropriate materials, and as further exemplified by the following specific examples. They may also be prepared by methods known per se, as described in the literature (for example in standard works, such as Houben-Weyl, Methoden der Organischen Chemie [Methods of Organic Chemistry], Georg Thieme Verlag, Stuttgart; Organic Reactions, John Wiley & Sons, Inc., New York), to be precise under reaction conditions which are known and suitable for the said reactions. Use can also be made of variants which are known per se, but are not mentioned here in greater detail.

Likewise, the starting materials for the preparation of compounds of the present invention can be prepared by methods as described in the examples or by methods known per se, as described in the literature of synthetic organic chemistry and known to the skilled person, or can be obtained commercially. The starting materials for the processes claimed and/or utilized may, if desired, also be formed in situ by not isolating them from the reaction mixture, but instead immediately converting them further into the compounds of the invention or intermediate compounds. On the other hand, in general it is possible to carry out the reaction stepwise.

Preferably, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, dimethylformamide (DMF) or N-methyl pyrrolidinone (NMP); nitriles, such as acetonitrile; sulfoxides, such as dimethyl sulfoxide (DMSO); nitro compounds, such as nitromethane or nitrobenzene; esters, such as ethyl acetate, or mixtures of the said solvents or mixtures with water.

The reaction temperature is between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present invention claimed herein can be readily prepared. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The present invention also refers to a process for manufacturing a compound according to formula (I), or derivatives, N-oxides, prodrugs, solvates, tautomers or stereoisomers thereof as well as the physiologically acceptable salts of each of the foregoing. This process is characterized in that (a) a compound of formula (II)

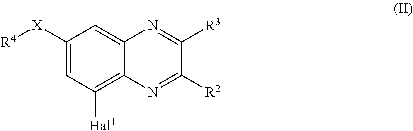

wherein
HaI¹ denotes Cl, Br or I;
R², R³, R⁴, X have the same meaning as defined hereinabove and in claims 1 to 10 for compounds of formula (I);
   is reacted under C—C coupling reaction conditions which conditions may utilize one or more suitable C—C coupling reaction reagents including catalysts
   with a compound R¹-RG^{a}
   wherein
R¹ has the same meaning as defined hereinabove and in claims 1 to 10 for compounds of formula (I);
RG^{a} denotes a chemical moiety being reactive under the particular C—C coupling reaction conditions utilized;
   or
   (b) a compound of formula (III)

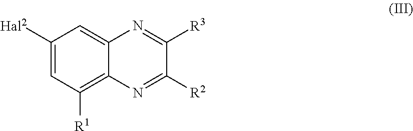

wherein
HaI² denotes Cl, Br or I;
R¹, R², R³ have the same meaning as defined hereinabove and in claims 1 to 10 for compounds of formula (I);
   is reacted under C—N coupling reaction conditions which conditions may utilize one or more suitable C—N coupling reaction reagents including catalysts
   with a compound R⁴—NHR⁵,
   wherein
R⁴, R⁵ have the same meaning as defined hereinabove and in claims 1 to 10 for compounds of formula (I);
   or
   (c) a compound of formula (III)

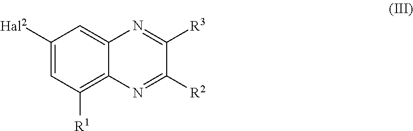

wherein
HaI² denotes Cl, Br or I;
R¹, R², R³ have the same meaning as defined hereinabove and in claims 1 to 10 for compounds of formula (I);
   is reacted under C—O coupling reaction conditions which conditions may utilize one or more suitable C—O coupling reaction reagents including catalysts
   with a compound R⁴—OH,
   wherein
R⁴ has the same meaning as defined hereinabove and in claims 1 to 10 for compounds of formula (I).

As will be understood by the person skilled in the art of organic synthesis compounds of the present invention, in particular compounds of formula (I), are readily accessible by various synthetic routes, some of which are exemplified in the accompanying Experimental Part. The skilled artisan will easily recognize which kind of reagents and reactions conditions are to be used and how they are to be applied and adapted in any particular instance—wherever necessary or useful—in order to obtain the compounds of the present invention. Furthermore, some of the compounds of the present invention can readily be synthesized by reacting other compounds of the present invention under suitable conditions, for instance, by converting one particular functional group being present in a compound of the present invention, or a suitable precursor molecule thereof, into another one by applying standard synthetic methods, like reduction, oxidation, addition or substitution reactions; those methods are well known to the skilled person. Likewise, the skilled artisan will apply whenever necessary or useful synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

A particularly versatile starting point for making compounds of formula (I) are 5-bromo-7-chloroquinoxaline (Int 2) and 7-bromo-5-chloroquinoxaline (Int 3) both of which are readily available by applying in analogy synthetic methods described in WO 2010/20363 A1.

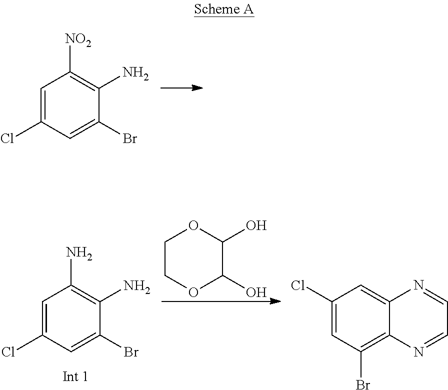

2-Bromo-4-chloro-6-nitrophenyl is converted into 3-bromo-5-chlorobenzene-1,2-diamino (Int 1) by utilizing suitable reduction means, e.g. tin(II)-chloride, which in turn is converted into 5-bromo-7-chloroquinoxaline (Int 2) by reacting it with 2,3-dihydroxy-1,4-dioxane.

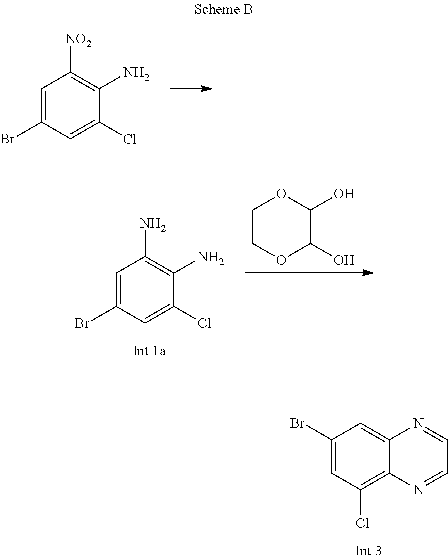 [SMILES: Nc1c(Cl)cc(Br)cc1[N+](=O)[O-] ; Nc1cc(Br)cc(Cl)c1N ; OC1OCCOC1O ; Clc1cc(Br)cc2nccnc12]

Likewise, 7-bromo-5-chloroquinoxaline (Int 3) is available by applying the same methodology under similar conditions (see Scheme B). It is to be noted that compounds of formula (I) in which either one or both substituents R² and R³ do not denote hydrogen, are available from precursor molecules similar to Int 2 and Int 3 by applying similar methods and optional purification/separation from isomers (see Scheme C):

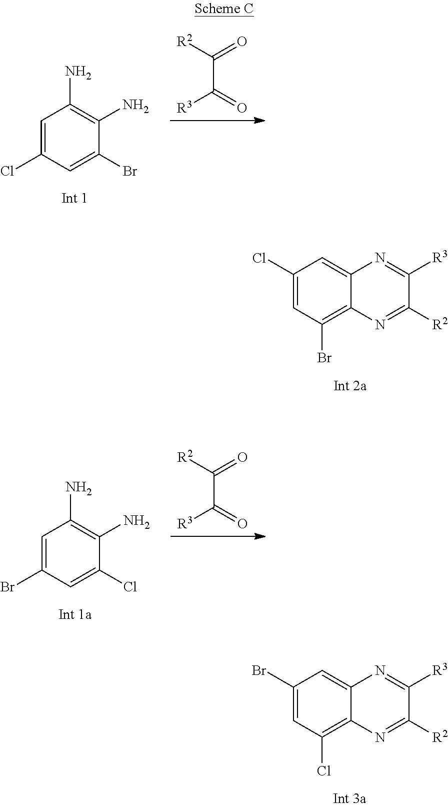

In one particular approach for making compounds of the present invention precursor molecule Int 2 (or Int 2a, as the case may be) is converted into a compound of formula (III) with HaI^{e} being bromine and R¹ being defined as in the description hereinabove and in the claims by applying either C—C coupling reaction conditions (if R¹ is connected to the quinoxaline system via a carbon atom) or C—N coupling reaction conditions (if R¹ is connected to the quinoxaline system via a nitrogen atom).

Typical suitable C—C coupling reactions are, among others, the Heck reaction, the Suzuki coupling, the Stille coupling, the Negishi coupling and coupling reactions utilizing organo cuprates, and well-known variants thereof. Depending on the specific method applied reagents, solvents and reaction conditions are selected accordingly. For instance, in case the introduction of R¹ is performed by utilizing Suzuki coupling conditions, precursor molecule Int 2 (or Int 2a) may be reacted with a suitable borate or boronate ester, (B(OSub)₃ with Sub being a suitable substituent, radical or residue (like trimethylborate or 4,4,5,5-tetramethyl-2-(tetramethyl-1,3,2-dioxaborolan-2-yl)-1,3,2-dioxaborolane) in the presence of an organometallic palladium (II) catalyst (like [1,1′-bis(diphenyl)phosphino)ferrocene]-dichloropalladium(II) dichloromethane complex) and optionally potassium acetate in order to form a derivative of Int 2 (or Int 2a) in which the bromine substituent is replaced by B(OH)₂ or B(OSub)₂, as the case may be; this derivative may then be reacted with a suitable halide R¹-HaI in the presence of a palladium(0) complex (e.g., tetrakis(triphenylphosphine)palladium(0)) and a base (e.g., sodium, potassium or cesium carbonate) to build a compound of formula (III). Similarly, the same compound of formula (III) can be obtained by forming a boron-substituted precursor R¹—B(OH)₂ or R¹—B(OSub)₂ and reacting it with Int 2 (or Int 2a) under similar conditions.

Likewise, C—N coupling reactions may be any suitable C—N coupling reaction of a heterocyclic system or a molecule bearing a reactive amino group with precursor molecule Int 2 (or Int 2a). Depending on the specific coupling reaction applied, it may well be that one or both of the reaction partners are subject to chemical transformation into intermediates before the reaction with the appropriate reaction partner occurs; for instance, the suitably substituted halide may be transformed into a respective boronic acid or boronic acid ester derivative before the reaction with the heterocyclic system or the reactive amine derivative occurs. Preferably, this coupling reaction is performed in the presence of a transition metal catalyst. Well-known examples of such C—N coupling reactions are, among others, the Hartwig-Buchwald reaction, the Ullmann coupling reaction, reactions similar to Suzuki or Heck reaction and coupling reactions utilizing organo cuprates. Depending on the specific method applied reagents, solvents and reaction conditions are selected accordingly.

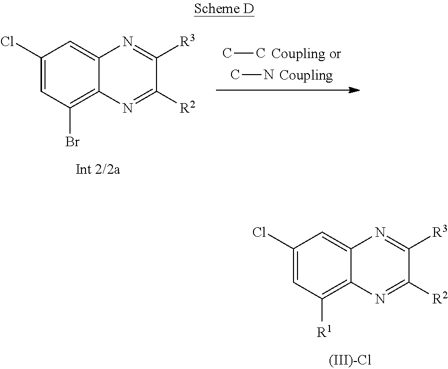

In order to obtain various compounds of formula (I) compounds of formula (III)-Cl obtained as shown in Scheme D may then be subjected to further synthetic modifications for introducing suitable functional groups that allow for, if required, still further modifications. One of these various methods is depicted in Scheme E showing the conversion of a compound of formula (III)-Cl into a compound of formula (IV)-NH₂, i.e., of a chloride into an amine, which may then be subjected to further reactions.

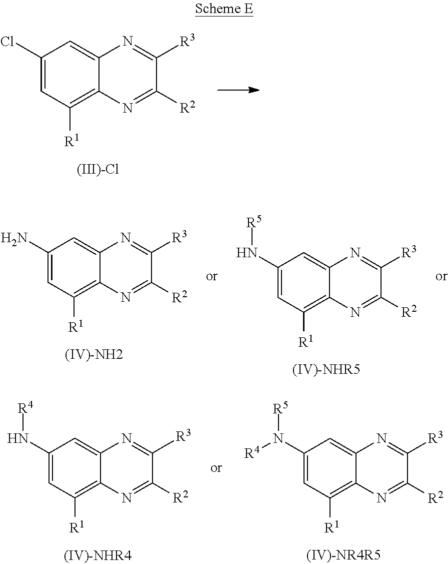

This functional group conversion to the amine (IV)-NH₂ may be achieved by subjecting the chloride (III)-Cl to a Hartwig-Buchwald reaction, i.e., by reacting it with ammonia (or an ammonia solution) in the presence of a palladium(II) catalyst, a suitable phosphine ligand and sodium tert.-butylate (e.g., Pd₂(dba)₃/Me₄tBuXPhos/NaOtBu/NH₃). If an amine R⁵—NH₂ (with R⁵ being as defined in the specification herein or in the claims and not being hydrogen) is used instead of ammonia (which could also be denoted as R⁵—NH₂ with R⁵ being H), compounds of formula (IV)-NHR⁵ may be obtained. Likewise, if amines NH₂R⁴ or NHR⁴R⁵ are utilized instead of ammonia or NH₂R⁵, the respective compounds of (IV)-NHR⁴ (which could also be described as a compound of formula (I) with X being NH) and (IV)-NR⁴R⁵ (which could also be described as a compound of formula (I) with X being NR⁵) are obtained. Other typical C—N coupling reactions, like those described above for Scheme D, may also be applied.

This methodology, i.e. reacting a compound of formula (III)-Cl with an amine NHR⁴R⁵ (with R⁴ and R⁵ being as defined hereinabove for formula (I)) under suitable C—N coupling reaction conditions, may be particular useful for the introduction of functionalized or rather complex substituents R⁴; it can be used to prepare compounds of formula (I) in which R⁴ denotes Ar^{W} or Hetar^{W} which are both substituted with R^{W1} in ortho-position and may be further substituted with R^{W2} and/or R^{W3} which are as defined hereinabove and in the claims. Depending on the very nature of R⁴, it may be introduced directly by reacting a compound of formula (III)-Cl with the amine NHR⁴R⁵; in some instances it may be preferable or even necessary to build up a particular substituent in stepwise manner. This approach is exemplified in Scheme F and can easily be adapted to different substitution pattern, where Ar^{W} is replaced by Hetar^{W}.

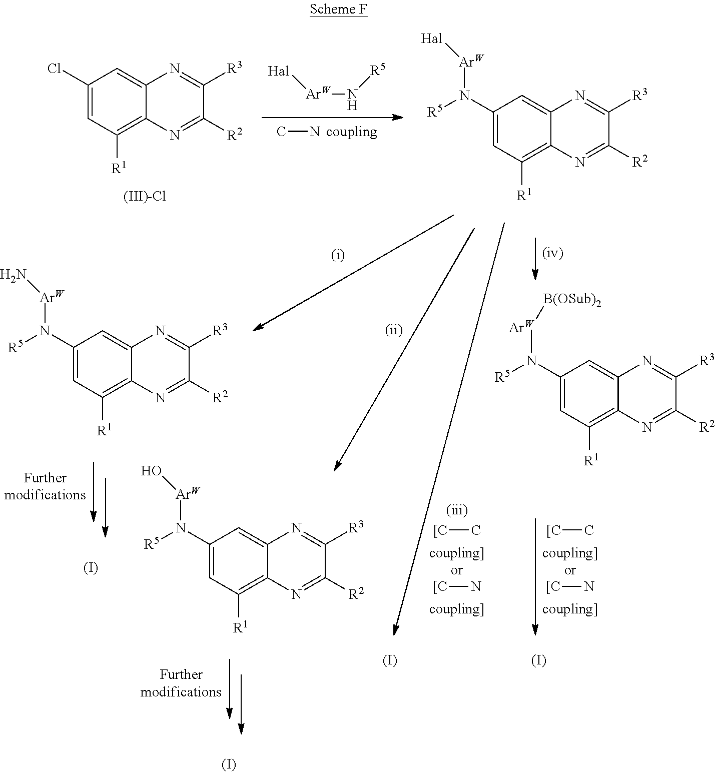

Similar to the conversion depicted in Scheme E, the halogen functional group can be converted to the respective amino group (see route (i)) by subjecting the halogen compound to a Hartwig-Buchwald reaction, i.e., by reacting it with ammonia in the presence of a palladium(II) catalyst, a suitable phosphine ligand and sodium tert-butylate (e.g., Pd₂(dba)₃/Me₄tBuXPhos/NaOtBu/NH₃). The amine thus obtained can subsequently be converted into other compounds of the present invention of formula (I). The conversion of the halogen functional group into a hydroxyl functional group (see route (ii) in Scheme F) can be effected, for instance, by applying a palladium(II) catalyst in the presence of a suitable phosphine and potassium hydroxide. Again, the hydroxyl-substituted compound thus obtained can subsequently be converted into other compounds of the present invention of formula (I).

According to reaction route (iii) of Scheme F, utilizing well-known C—C coupling or C—N coupling reactions yields still further compounds of the present invention. Typical suitable C—C coupling reactions that can be applied are, among others, the Heck reaction, the Suzuki coupling, the Stille coupling, the Negishi coupling and coupling reactions utilizing organo cuprates, and well-known variants thereof. Depending on the specific method applied reagents, solvents and reaction conditions are selected accordingly. For instance, in case the introduction of a Hetar^{Y} residue is performed by utilizing Suzuki coupling conditions, the halogen-substituted compound depicted in Scheme F may be reacted with a suitable Hetar^{Y} boronate (Hetar^{Y}-B(OH)₂ or Hetar^{Y}-B(0Sub)₂ (with Sub being a suitable substituent)) in the presence of an organometallic palladium (II) catalyst (like [1,1′-bis(diphenyl)phosphino)ferrocene]-dichloropalladium(II) dichloromethane complex) and optionally potassium acetate in order to form a compound of formula (I) in which R⁴ denotes Ar^{W}—R^{W1} with R^{W1} being Hetar^{Y}. Likewise, an appropriate C—N coupling reaction may be any suitable C—N coupling reaction of a heterocyclic system or a molecule bearing a reactive amino group with the halogen-substituted compound shown in Scheme F. Depending on the specific coupling reaction applied, it may well be that one or both of the reaction partners are subject to chemical transformation into intermediates before the reaction with the appropriate reaction partner occurs. Preferably, this coupling reaction is performed in the presence of a transition metal catalyst. Well-known examples of such C—N coupling reactions are, among others, the Hartwig-Buchwald reaction, the Ullmann coupling reaction, reactions similar to Suzuki or Heck reaction and coupling reactions utilizing organo cuprates. Depending on the specific method applied reagents, solvents and reaction conditions are selected accordingly.

Similar C—C couplings or C—N couplings, as the case may be, can be utilized, when synthetic approach (iv) of Scheme F is applied: Here the halogen-substituted compound of Scheme F is converted into a suitable boronic acid or boronic acid ester precursor which is then reacted, typically in the presence of a palladium(II) catalyst, an appropriate phosphine ligand and a base, with a bromine or chlorine substituted reaction partner (e.g., Ar^{Y}—Br, Hetar^{Y}-Br, Hetcyc^{Y}-Br) to afford the respective compound of formula (I).

Compounds of formula (IV)-NH₂ or (IV)-NHR₅ or (IV)-NHR₄, obtainable according to Scheme E, may also be the starting point for obtaining compounds of formula (I) with X being N—R⁵ (with R⁵ being as defined in the specification hereinabove or in the claims) by applying suitable C—N coupling reactions with compounds of formula R⁴-HaI or R⁵-HaI, as the case may be.

Another approach for making compounds of the present invention of formula (I) utilizes one of the above-mentioned precursors Int 3 and Int 3a. By applying one of the C—N coupling methodologies already described in some detail hereinabove Int 3 (or Int 3a) can be converted into a compound of formula (II) with HaI¹ being Cl and X being NH (Scheme G):

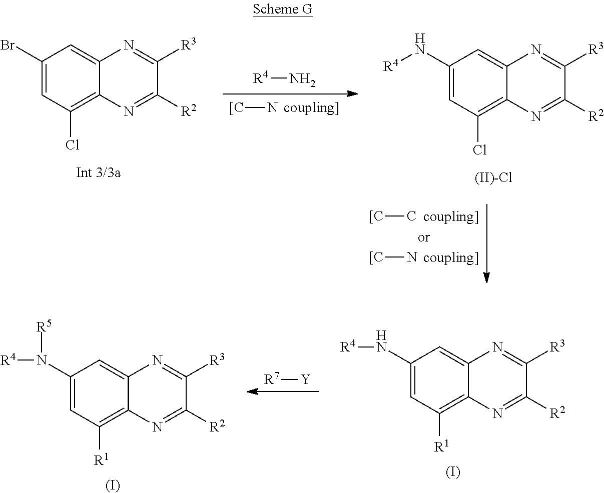

Replacing the chlorine substituent of compound (II)-Cl by substituent R¹ can then be effected by utilizing similar reaction methods already described above for making compounds of formula (III)-Cl (Scheme D), i.e. C—C coupling or C—N coupling reactions described herein. Introduction of a substituent R⁵ not being hydrogen can be effected, e.g., by nucleophilic substitution with a suitable reaction partner R⁵—Y (Y being an appropriate leaving group). Alternatively, the moiety R⁵ not being hydrogen may be introduced by utilizing a suitably substituted amine R⁴NHR⁵ in the C—N coupling reaction with Int 3 or Int 3a.

Compounds of formula (I) with X denoting O (oxygen) are available by the synthetic route depicted in Scheme H:

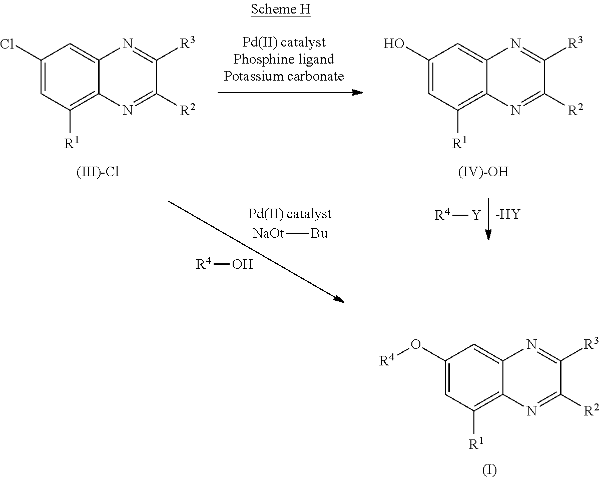

A compound of formula (III)-Cl may be converted into the respective hydroxyl-substituted compound of formula (IV)-OH by utilizing a suitable palladium(II) catalyst in the presence of an appropriate phosphine ligand and K₂CO₃. The hydroxyl compound (IV)-OH can then be reacted with a compound of formula R⁴—Y (with Y being a typical leaving group) under conditions that are usually applied for nucleophilic substitution reactions to afford the compound of formula (I). Alternatively, a compound of formula (III)-Cl may directly converted into the respective compound of formula (I) by reacting it with the alcohol R⁴—OH under palladium(II)/phosphine ligand catalysis in the presence of sodium tert-butylate.

### EXPERIMENTAL PART

### Abbreviations

Some abbreviations that may appear in this application are defined as follows hereinafter:

| Abbreviation | Meaning |
|---|---|
| Aq. | aqueous |
| (Pd(cinnamyl)Cl)₂ | Palladium(TT-cinnamyl) chloride dimer |
| BINAP | (±)-2,2′-Bis(diphenylphosphino)-1,1′- |
| | binaphthalene |
| Boc | tert-butoxycarbonyl |
| BippyPhos | 5-(Di-tert-butylphosphino)-1′,3′,5′- |
| | triphenyl-1′H-[1,4′]bipyrazole |
| BrettPhos | 2-(Dicyclohexylphosphino)3,6-dimethoxy- |
| | 2′,4′,6′-triisopropyl-1,1′-biphenyl |
| BrettPhos precatalyst | Chloro[2-(dicyclohexylphosphino)-3,6- |
| | dimethoxy-2′,4′,6′-triisopropyl-1,1′- |
| | biphenyl][2-(2- |
| | aminoethyl)phenyl]palladium(II) |
| DBU | 1,8-Diazabicyclo[5.4.0]undec-7-ene |
| DCM | Dichloromethane |
| DIPEA | N,N-Diisopropylethylamine |
| DMAP | 4-Dimethylaminopyridine |
| DME | 1,2-Dimethoxyethane |
| DMF | N,N-Dimethylformamide |
| DMSO | Dimethyl sulfoxide |
| DMTMM | 4-(4,6-Dimethoxy-1,3,5-triazin-2-yl)-4- |
| | methylmorpholinium chloride |
| EDC•HCl | (3-Dimethylamino-propyl)- |
| | ethylcarbodiimide hydrochloride |
| EtOAc | Ethyl acetate |
| FCC | Flash column chromatography |
| Hantzsch ester | Diethyl 1,4-dihydro-2,6-dimethyl-3,5- |
| | pyridinedicarboxylate |
| HATU | 1-[Bis(dimethylamino)methylene]-1H-1,2,3- |
| | triazolo[4,5-b]pyridinium 3-oxid |
| | hexafluorophosphate |
| Herrmann's palladacycle | trans-Di(μ-acetato)bis[o-(di-o-tolyl- |
| | phosphino)benzyl]dipalladium(II), trans-Di- |
| | μ-acetatobis[2-[bis(2- |
| | methylphenyl)phosphino]benzyl]dipalladium |
| HOAt | 1-Hydroxy-7-azabenzotriazole |
| HOBt | 1-Hydroxybenzotriazole |
| HPLC | High-performance liquid chromatography |
| tBuOK (KOtBu) | Potassium tert-butoxide |
| KOAc | potassium acetate |
| LiHMDS | Lithium bis(trimethylsilyl)amide solution |
| mCPBA | 3-Chloroperoxybenzoic acid |
| Me4-t-ButylXphos | 2-Di-tert-butylphosphino-3,4,5,6- |
| | tetramethyl-2′,4′,6′-triisopropyl-1,1′- |
| | biphenyl |
| μW | Microwave |
| NaBH(OAc)₃ | Sodium triacetoxyborohydride |
| tBuONa (NaOtBu) | Sodium tert-butoxide |
| NMP | 1-Methyl-2-pyrrolidinone |
| OXONE | Potassium peroxymonosulfate (linear |
| | formula: KHSO₅•0.5KHSO₄•0.5K₂SO₄) |
| Palau′Chlor | 2-Chloro-1,3- |
| | bis(methoxycarbonyl)guanidine |
| Pd(dppf)Cl₂ | [1,1′- |
| | Bis(diphenylphosphino)ferrocene]dichloropalladium(II) |
| Pd(dppf)Cl₂•CH₂Cl₂ | [1,1′- |
| | Bis(diphenylphosphino)ferrocene]dichloropalladium(II), |
| | complex with dichloromethane |
| Pd(OAc)₂ | palladium(II) acetate |
| Pd(PPh₃)₄ | tetrakis(triphenylphosphine)palladium(0) |
| Pd₂(dba)₃ | Tris(dibenzylideneacetone)dipalladium(0) |
| PTSA | p-toluenesulfonic acid monohydrate |
| RM | reaction mixture |
| RT | Room temperature |
| sat. | saturated |
| tBuXPhos Pd G3 | [(2-di-tert-butylphosphino-2′,4′,6′- |
| | triisopropyl-1,1′-biphenyl)-2-(2′-amino-1,1′- |
| | biphenyl)] palladium(II) methanesulfonate |
| t-BuBrettPhos | 2-(Di-tert-butylphosphino)-2′,4′,6′- |
| | triisopropyl-3,6-dimethoxy-1,1′-biphenyl |
| TEA | Triethylamine |
| TFA | Trifluoroacetic acid |
| THF | Tetrahydrofurane |
| TMSCl | Chlorotrimethylsilane |
| Xantphos | 4,5-Bis(diphenylphosphino)-9,9- |
| | dimethylxanthene |
| Xphos | 2-Dicyclohexylphosphino-2′,4′,6′- |
| | triisopropylbiphenyl |

All anhydrous solvents are provided by commercial suppliers, e.g., Sigma-Aldrich®, in appropriate containers, e.g., Sure/Seal™ bottles, and are used without further purification.

The compounds of the present invention can be prepared according to the procedures of the following Schemes and Examples, using appropriate materials and are further exemplified by the following specific examples. Analytical data of compounds made according to the following examples are shown in Table 1.

The invention will be illustrated, but not limited, by reference to the specific embodiments described in the following examples. Unless otherwise indicated in the schemes, the variables have the same meaning as described above and in the claims.

Unless otherwise specified, all starting materials are obtained from commercial suppliers and used without further purifications. Unless otherwise specified, all temperatures are expressed in ° C. and all reactions are conducted at RT. Compounds are purified by either silica chromatography or preparative HPLC. Unless otherwise specified, silica is the stationary phase used for flash column chromatography purifications.

### ¹H NMR:

¹H NMR is recorded on 400 MHz spectrometers. Chemical shifts (6) are reported in ppm relative to the residual solvent signal (δ=2.5 ppm for ¹H NMR in DMSO-d6). ¹H NMR data are reported as follows: chemical shift (multiplicity, coupling constants and number of hydrogens). Multiplicity is abbreviated as follows: s (singlet), d (doublet), t (triplet), q (quartet), m (multiplet), dd (doublet of doublets), tt (triplet of triplets), td (triplet of doublets) br (broad).

NMR, UPLC, HPLC and MS data provided in the examples described below are registered on:

NMR: Bruker Avance III HD 400 MHz, probe BBO

### LC-MS Analyses on Bruker Amazon SL

Method name: Ic-ms1-2-ba

### Equipment:

### MS Bruker Amazon SL

### LC Dionex Ultimate 3000

HPLC with UV-Vis or DAD detector
Column: Waters Acquity UPLC HSS C18, 50 mm×2.1 mm×1.8 μm

### Eluents:

(A) 0.1% formic acid in ACN
(B) 0.1% formic acid in water

### Analytical Method:

Autosampler: injection volume: 1 μL

### Pump:

| Time | Flow | |
|---|---|---|
| [min] | [ml/min] | % B |
| 0.00 | 0.5 | 95 |
| 0.00 | 0.5 | 95 |
| 4.00 | 0.5 | 5 |
| 5.00 | 0.5 | 5 |
| 5.20 | 0.5 | 95 |
| 6.00 | 0.5 | 95 |

Column compartment:
Column temperature: 25° C.
Time of analysis: 6 min

### Detector:

### Wavelength: 254, 230, 270, 280 nm

### Bruker Amazon SL

Method name: BCM-30

### Equipment:

### MS Bruker Amazon SL

### LC Dionex Ultimate 3000

HPLC with UV-Vis or DAD detector

### Column: Waters Symmetry C18 3.9×150 mm 5 μm

### Eluents:

(A) 0.1% formic acid-water solution
(B) 0.1% formic acid-ACN solution

### Analytical Method:

Autosampler: injection volume: 3 μL

### Pump:

Flow: 1.2 ml/min

| Time | |
|---|---|
| [min] | [%] B |
| 0.0 | 20 |
| 20.0 | 80 |
| 22.0 | 80 |
| 22.5 | 95 |
| 25.0 | 95 |
| 25.3 | 20 |
| 30.0 | 20 |

### Column Compartment:

Column temperature: 25° C.
Time of analysis: 30 min

### Detector:

Wave length: 254 nm
Method name: Kinetex-BCM

### Equipment:

HPLC with UV-Vis or DAD detector

### Column: Kinetex XB C18 4.6×50 mm 2.6 μm

### Eluents:

(A) 0.1% formic acid-water solution
(B) 0.1% formic acid-ACN solution

### Analytical Method:

Autosampler: injection volume: 1 μL

### Pump:

Flow: 0.5 mL/min

| Time | |
|---|---|
| [min] | [%] B |
| 0.0 | 20 |
| 6.7 | 80 |
| 7.5 | 80 |
| 7.8 | 95 |
| 9.5 | 95 |
| 10.0 | 20 |
| 12.0 | 20 |

### Column Compartment:

Column temperature: 25° C.
Time of analysis: 12 min

### Detector:

### DAD

### Shimadzu LC-MS:

Method name: lc-ms1-2-ba

### Equipment:

### Shimadzu UPLC-MS 2020

HPLC with UV-Vis or DAD detector
Column: Waters Acquity UPLC HSS C18, 50 mm×2.1 mm×1.8 μm

### Eluents:

(A) 0.1% formic acid in ACN
(B) 0.1% formic acid in water

### Analytical Method:

Autosampler: injection volume: 1 μL

### Pump:

| Time | Flow | |
|---|---|---|
| [min] | [ml/min] | % B |
| 0.00 | 0.5 | 95 |
| 0.00 | 0.5 | 95 |
| 4.00 | 0.5 | 5 |
| 5.00 | 0.5 | 5 |
| 5.20 | 0.5 | 95 |
| 6.00 | 0.5 | 95 |

### Column Compartment

Column temperature: 25° C.
Time of analysis: 6 min

### Detector:

Wave length: 254, 230, 270, 280 nm

### Corona Ultra:

Method name: BCM-30

### Equipment:

Corona ultra

### LC Dionex Ultimate 3000

### Column: Waters Symmetry C18 3.9×150 mm 5 μm

### Eluents:

(A) 0.1% formic acid-water solution
(B) 0.1% formic acid-ACN solution

### Analytical Method:

Autosampler: injection volume: 3μ

### Pump:

Flow: 1.2 ml/min

| Time | |
|---|---|
| [min] | [%] B |
| 0.0 | 20 |
| 20.0 | 80 |
| 22.0 | 80 |
| 22.5 | 95 |
| 25.0 | 95 |
| 25.3 | 20 |
| 30.0 | 20 |

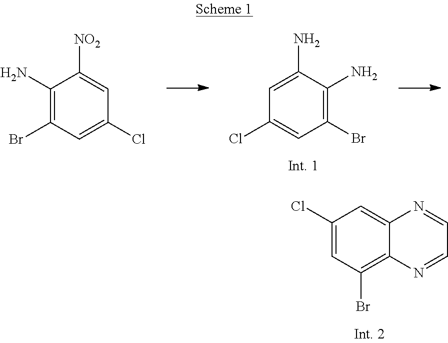 [SMILES: Nc1c(Br)cc(Cl)cc1[N+](=O)[O-] ; Nc1cc(Cl)cc(Br)c1N ; Clc1cc(Br)c2nccnc2c1]

### Intermediate 1 (acc. to: US2013/116262 A1)

### 3-Bromo-5-chlorobenzene-1,2-diamine

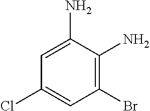

To a stirred solution of tin(I1)chloride dihydrate (53.8 g; 238 mmol; 6 eq.) in EtOAc (400 mL), 2-bromo-4-chloro-6-nitrophenylamine (10 g; 39.8 mmol; 1 eq.) is added in three portions. The reaction mixture is refluxed for 2 h. The dry residue obtained after evaporation of the solvent is suspended in DCM (1 L) and treated with aq. NaOH (˜300 mL, 10 M, >50 eq.). The mixture is stirred for 4 h and the layers are separated. The organic layer is washed with water and brine, dried over anhydrous Na₂SO₄, filtered and concentrated to provide 3-bromo-5-chlorobenzene-1,2-diamine (8.4 g; yield: 95%; beige solid; UPLC purity: 97%).

### Intermediate 2 (acc. to: WO2010/20363 A1)

### 5-Bromo-7-chloroquinoxaline

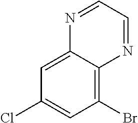 [SMILES: Clc1cc(Br)c2nccnc2c1]

3-Bromo-5-chloro-1,2-diaminobenzene (Intermediate 1, 8.4 g; 37.9 mmol; 1 eq.) is dissolved in EtOH (250 mL) and 2,3-dihydroxy-1,4-dioxane (4.5 g, 37.9 mmol; 1 eq.) is added. The mixture is stirred for 4 h at room temperature and a second portion of 2,3-dihydroxy-1,4-dioxane (2.3 g; 18.9 mmol; 0.5 eq.) is added. After stirring for 24 h at room temperature, the precipitate is filtered off, washed with ethanol and dried in vacuo to give 5-bromo-7-chloroquinoxaline as a beige solid (6.71 g; yield: 74%; UPLC purity: 96%).

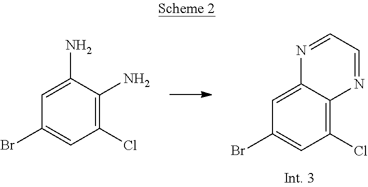 [SMILES: Nc1cc(Br)cc(Cl)c1N ; Clc1cc(Br)cc2nccnc12]

### Intermediate 3 (Acc. to: WO2010/20363 A1)

### 7-Bromo-5-chloroquinoxaline

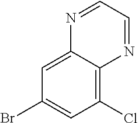 [SMILES: Clc1cc(Br)cc2nccnc12]

5-Bromo-3-chloro-1,2-diaminobenzene (4.6 g; 20 mmol; 1 eq.) is dissolved in EtOH (200 mL) and 2,3-dixydroxy-1,4-dioxane (2.5 g, 20 mmol; 1 eq.) is added. The mixture is stirred for 4 h at room temperature and a second portion of 2,3-dihydroxy-1,4-dioxane (1.3 g; 10 mmol; 0.5 eq.) is added. After stirring for 24 h at room temperature, the reaction mixture is concentrated and the residue is purified by FCC (EtOAc gradient in hexane) to provide 7-bromo-5-chloroquinoxaline as a beige solid (4.7 g; yield: 92%; UPLC purity: 98%).

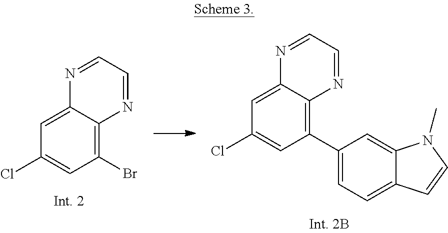 [SMILES: Clc1cc(Br)c2nccnc2c1 ; Cn1ccc2ccc(-c3cc(Cl)cc4nccnc34)cc21]

### Intermediate 2B

### 7-Chloro-5-(1-methyl-1H-indol-6-yl)quinoxaline

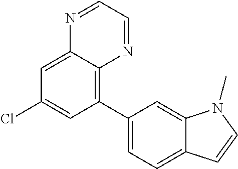 [SMILES: Cn1ccc2ccc(-c3cc(Cl)cc4nccnc34)cc21]

A pressure vessel or sealed tube is charged with 5-bromo-7-chloroquinoxaline (Intermediate 2, 3 g; 12.2 mmol; 1 eq.), 1-methyl-6-(4,4,5,5,-tetramethyl-1,3,2-dioxaborolan-2-yl)-1H-indole (2.5 g; 9.8 mmol; 1 eq.), DIPEA (3.2 g; 24.4 mmol; 2 eq.), dioxane (16 mL) and water (16 mL). The suspension is deoxygenated by bubbling with argon and Pd(dppf)Cl₂ (0.89 g; 1.22 mmol; 0.1 eq.) is added. The reaction tube is sealed and the reaction mixture is stirred at 85° C. for 3 h. The mixture is filtered through a Celite pad and the filtrate is diluted with DCM. The organic phase is washed with water and brine, dried over Na₂SO₄, filtered and concentrated in vacuo. The residue is purified by FCC (EtOAc gradient in hexane) to afford 7-chloro-H-indol-6-yl)quinoxaline (2.2 g; yield: 56%; UPLC purity: 92%) as a yellow solid.

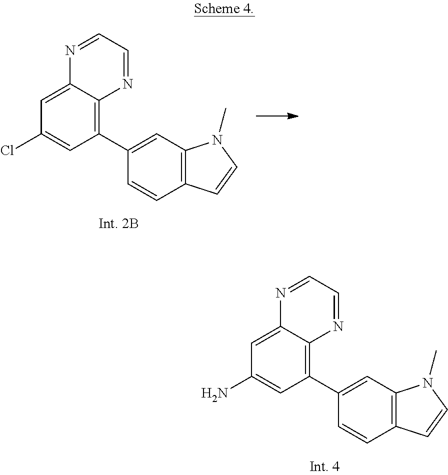 [SMILES: Cn1ccc2ccc(-c3cc(Cl)cc4nccnc34)cc21 ; Cn1ccc2ccc(-c3cc(N)cc4nccnc34)cc21]

### Intermediate 4

### 8-(1-Methyl-1H-indol-6-yl)quinoxalin-6-amine

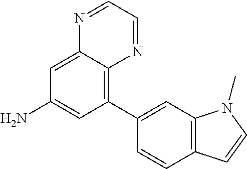 [SMILES: Cn1ccc2ccc(-c3cc(N)cc4nccnc34)cc21]

A pressure vessel is charged with 7-chloro-5-(1-methyl-1H-indol-6-yl)-quinoxaline (Intermediate 2B, 100 mg; 0.31 mmol; 1 eq.), Pd₂(dba)₃ (29 mg; 0.03 mmol; 0.1 eq.), Me₄-tBuXPhos (15 mg; 0.03 mmol; 0.1 eq.) and tBuONa (42 mg; 0.44 mmol; 1.4 eq.). The tube's atmosphere is then evacuated and backfilled with argon (three times). An ammonia solution (0.5 M in dioxane, 12.60 mL; 6.30 mmol; 20 eq.) is added via syringe and the reaction mixture is stirred at 80° C. for 5 h. The crude product is purified by FCC (0-5% MeOH gradient in DCM) to afford 8-(1-methyl-1H-indol-6-yl)quinoxalin-6-amine (70 mg; yield: 78%; yellow powder; UPLC purity: 96%).

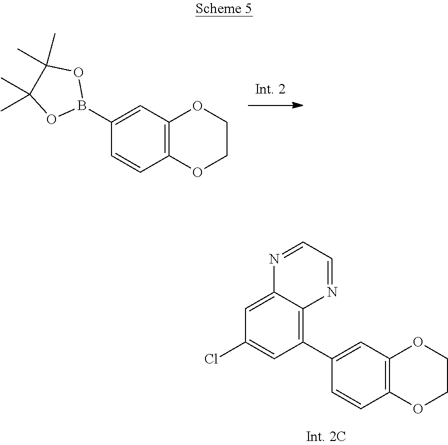 [SMILES: CC1(C)OB(c2ccc3c(c2)OCCO3)OC1(C)C ; Clc1cc(-c2ccc3c(c2)OCCO3)c2nccnc2c1]

### Intermediate 2C

### 7-Chloro-5-(2,3-dihydro-1,4-benzodioxin-6-yl)quinoxaline

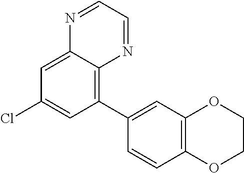 [SMILES: Clc1cc(-c2ccc3c(c2)OCCO3)c2nccnc2c1]

A pressure vessel is charged with 5-bromo-7-chloroquinoxaline (150 mg; 0.59 mmol; 1 eq.), 2-(2,3-dihydro-1,4-benzodioxin-6-yl)-4,4,5,5-tetramethyl-1,3,2-dioxaborolane (158 mg; 0.59 mmol; 1 eq.), cesium carbonate (389 mg; 1.18 mmol; 2 eq.), 1,2-dimethoxyethane (4 mL) and water (2 mL). The reaction mixture is deoxygenated by bubbling argon under sonication. Then Pd(dppf)Cl₂CH₂Cl₂ (74 mg; 0.09 mmol; 0.15 eq.) is added to the reaction mixture. After being flushed with argon, the tube is sealed and the reaction mixture is stirred for 1 h at 100° C., at which point UPLC analysis showed complete conversion of the starting material. The reaction mixture is cooled to room temperature and diluted with EtOAc. Phases are separated and the aq. layer is extracted with EtOAc. The combined organic phases are washed with water and brine, then dried over Na₂SO₄ and filtered through a pad of celite. The filtrate is concentrated in vacuo and the resulting residue is purified by FCC (0-40% EtOAc gradient in hexane) to give 7-chloro-5-(2,3-dihydro-1,4-benzodioxin-6-yl)quinoxaline (100 mg; 0.33 mmol; yield 57%; UPLC purity: 100%).

### Example 1, General Procedure 1

### 8-(2,3-Dihydro-1,4-benzodioxin-6-yl)-N-(4-methanesulfonylpyridin-3-yl)quinoxalin-6-amine

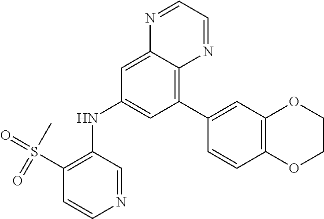 [SMILES: CS(=O)(=O)c1ccncc1Nc1cc(-c2ccc3c(c2)OCCO3)c2nccnc2c1]

A pressure vessel is charged with 7-chloro-5-(2,3-dihydro-1,4-benzodioxin-6-yl)quinoxaline (Intermediate 2C, 50 mg; 0.17 mmol; 1 eq.), 4-methanesulfonylpyridin-3-ylamine hydrochloride (44 mg; 0.20 mmol; 1.2 eq.) and cesium carbonate (5 eq.) in dioxane (1 mL). The reaction mixture is deoxygenated by bubbling argon under sonication. Then BINAP (11 mg; 0.02 mmol; 0.1 eq.) and Pd(OAc)₂ (4 mg; 0.02 mmol; 0.1 eq.) are added to the reaction mixture under argon. The tube is sealed and the reaction mixture is stirred for 60 min at 150° C. to achieve full conversion of starting material (Intermediate C). The reaction mixture is partitioned between EtOAc and water and the aqueous layer is extracted with EtOAc. The combined organic phases are washed with aq. saturated NaHCO₃ and brine, dried over sodium sulfate and filtered through a pad of celite. The filtrate is concentrated in vacuo to yield a yellow residue which is purified by FCC (EtOAc gradient in hexane) to afford 8-(2,3-dihydro-1,4-benzodioxin-6-yl)-N-(4-methanesulfonylpyridin-3-yl) quinoxalin-6-amine (60 mg; 0.14 mmol; yield: 81%; yellow powder; UPLC purity 98.7%).

### Example 2

### 5-(1-Methyl-1H-indol-6-yl)-7-{1H,2H,3H-pyrrolo[2,3-c]pyridin-1-yl}quinoxaline

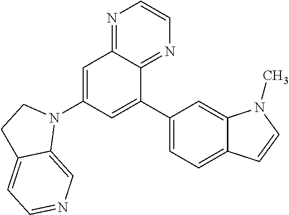 [SMILES: Cn1ccc2ccc(-c3cc(N4CCc5ccncc54)cc4nccnc34)cc21]

The title compound is prepared according to General Procedure 1 described in Example 1, with 7-chloro-5-(1-methyl-1H-indol-6-yl)quinoxaline (Intermediate 2B, 60 mg; 0.20 mmol; 1 eq.), 2,3-dihydro-1H-pyrrolo[2,3-c]pyridine hydrochloride (62 mg, 0.3 mmol, 1.5 eq.), cesium carbonate (399 mg; 1.21 mmol; 5 eq.), BINAP (13 mg; 0.02 mmol; 0.10 eq.) and Pd(OAc)₂ (5 mg; 0.02 mmol; 0.1 eq.) in dioxane (8 mL). Conditions: 150° C., 1 h. Purification by FCC (10% to 100% EtOAc gradient in hexane followed by 0% to 5% MeOH gradient in EtOAc) affords 5-(1-methyl-1H-indol-6-yl)-7-{1H,2H,3H-pyrrolo[2,3-c]pyridin-1-yl}quinoxaline (lot 1: 6 mg; 0.02 mmol; yield 8%; yellow powder; UPLC purity 98%) The rest of the product is isolated as a mixture with 2,3-dihydro-1H-pyrrolo[2,3-c]pyridine. It is triturated first with hexane, then with MeOH to afford 5-(1-methyl-1H-indol-6-yl)-7-{1H,2H,3H-pyrrolo[2,3-c]pyridin-1-yl}quinoxaline (Lot 2: 23 mg; 0.06 mmol; yield 30%; yellow powder; UPLC purity: 99%).

### Example 3 General Procedure 1A

### N-(2-Methanesulfonylphenyl)-8-(1-methyl-1H-indol-6-yl)quinoxalin-6-amine

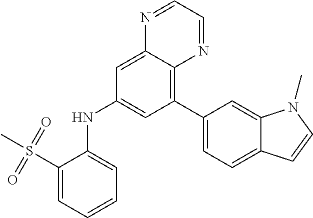 [SMILES: Cn1ccc2ccc(-c3cc(Nc4ccccc4S(C)(=O)=O)cc4nccnc34)cc21]

A pressure vessel is charged with 7-chloro-5-(1-methyl-1H-indol-6-yl)-quinoxaline (Intermediate 2B, 40 mg; 0.13 mmol; 1 eq.), 2-methanesulfonylphenylamine hydrochloride (64 mg; 0.31 mmol; 2.4 eq.), tBuONa (37 mg; 0.39 mmol; 3 eq.), BINAP (16 mg; 0.03 mmol; 0.2 eq.) and toluene (4 mL). The reaction mixture is flushed with argon and Pd₂(dba)₃ (30 mg; 0.01 mmol; 0.1 eq.) is added. The reaction vessel is sealed and the reaction mixture is stirred under microwave irradiation at 160° C. for 1 h. The residue obtained after solvent evaporation is purified by FCC (EtOAc gradient in hexane) to afford N-(2-methanesulfonylphenyl)-8-(1-methyl-1H-indol-6-yl)quinoxalin-6-amine (18 mg; 0.04 mmol; yield: 31%; yellow amorphous powder; HPLC purity: 95.8%).

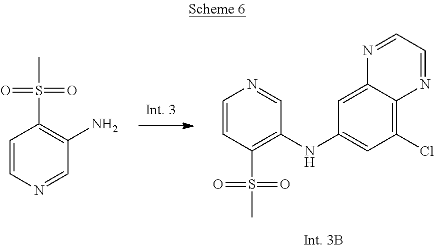 [SMILES: CS(=O)(=O)c1ccncc1N ; CS(=O)(=O)c1ccncc1Nc1cc(Cl)c2nccnc2c1]

### Intermediate 3B

### 8-Chloro-N-(4-methanesulfonylpyridin-3-yl)quinoxalin-6-amine

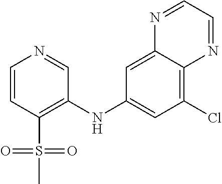 [SMILES: CS(=O)(=O)c1ccncc1Nc1cc(Cl)c2nccnc2c1]

In a pressure tube, 7-bromo-5-chloro-quinoxaline (Intermediate 3, 150 mg; 0.62 mmol; 1 eq.), 4-methanesulfonylpyridin-3-ylamine (127 mg; 0.74 mmol; 1.2 eq.), BINAP (76 mg; 0.12 mmol; 0.20 eq.) and cesium carbonate (802 mg; 2.46 mmol; 4 eq.) are suspended in dioxane (6 mL). The mixture is degassed by argon bubbling and sonication. Pd(OAc)₂ (14 mg; 0.06 mmol; 0.1 eq.) is added, the flask is sealed and the mixture is stirred at 100° C. for 0.5 h, at which point TLC showed complete conversion. The mixture is filtered on a pad of celite, eluting with DCM. The filtrate is washed with brine and concentrated. The residue is combined with another lot of title compound obtained the same way from 7-bromo-5-chloro-quinoxaline (50 mg; 0.21 mmol; 1 eq.), 4-methanesulfonylpyridin-3-ylamine (45 mg; 0.25 mmol; 1.2 eq.), BINAP (26 mg; 0.04 mmol; 0.2 eq.), cesium carbonate (268 mg; 0.82 mmol; 4 eq.) and Pd(OAc)₂ (5 mg; 0.02 mmol; 0.1 eq.) in dioxane (2 mL). The resulting mixture is triturated in acetone (4 mL), and the solid is filtered off. Trituration of this solid in hexane followed by filtration and drying gives 8-chloro-N-(4-methanesulfonylpyridin-3-yl)quinoxalin-6-amine (147 mg; 0.43 mmol; 71%; UPLC purity: 99%).

### Example 4

### 8-(1,3-Benzothiazol-6-yl)-N-(4-methanesulfonylpyridin-3-yl)quinoxalin-6-amine

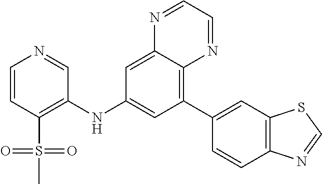 [SMILES: CS(=O)(=O)c1ccncc1Nc1cc(-c2ccc3ncsc3c2)c2nccnc2c1]

A 5-mL microwave vessel is charged with 8-chloro-N-(4-methanesulfonylpyridin-3-yl)quinoxalin-6-amine (Intermediate 3B, 80 mg; 0.24 mmol; 1 eq.), 6-(4,4,5,5-tetramethyl-[1,3,2]dioxaborolan-2-yl)-benzothiazole (75 mg; 0.29 mmol; 1.20 eq.), dioxane (3 mL) and a sodium carbonate 2 M aq. solution (0.24 mL; 0.48 mmol; 2 eq.). The mixture is degassed by sonication and bubbling argon for 10 min. Tetrakis(triphenylphosphine)palladium(0) (29 mg; 0.02 mmol; 0.10 eq.) is added and the vessel is sealed. The reaction mixture is heated under microwave irradiation at 180° C. for 30 minutes. The reaction mixture is filtered through a pad of celite and the filtrate is diluted with DCM. The organic phase is washed with water and brine, dried over Na₂SO₄ and concentrated. The residue is purified by FCC (0-5% MeOH in DCM) and by FCC (0-5% MeOH in EtOAc) to give 8-(1,3-benzothiazol-6-yl)-N-(4-methanesulfonylpyridin-3-yl)quinoxalin-6-amine (16 mg; 0.04 mmol; yield: 15%; bright yellow powder; HPLC purity: 97.6%).

### Example 5

### 8-(2-Chloro-5-methoxyphenyl)-N-(4-methanesulfonylpyridin-3-yl)quinoxalin-6-amine

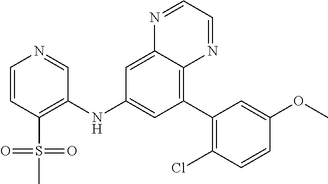 [SMILES: COc1ccc(Cl)c(-c2cc(Nc3cnccc3S(C)(=O)=O)cc3nccnc23)c1]

A microwave vial is charged with dioxane (2 mL), water (0.2 mL), 2-dicyclohexylphosphino-2′,6′-dimethoxybiphenyl (SPhos, 12 mg; 0.03 mmol; 0.20 eq.), 8-chloro-N-(4-methanesulfonylpyridin-3-yl)quinoxalin-6-amine (Intermediate 3B, 50 mg; 0.15 mmol; 1 eq.), potassium phosphate tribasic (127 mg; 0.60 mmol; 4 eq.), Pd(OAc)₂ (7 mg; 0.03 mmol; 0.20 eq.) and 2-chloro-5-methoxyphenylboronic acid pinacol ester (120 mg; 0.45 mmol; 3 eq.). The vial is capped, degassed, flushed with argon and heated under microwave irradiation for 20 minutes at 130° C. The reaction mixture is filtered over a pad of celite, eluting with DCM. The filtrate is washed with water and brine, dried over sodium sulfate, filtered and concentrated. The residue is purified by FCC (0% to 5% MeOH gradient in DCM), and followed by reversed-phase preparative HPLC (column: Gemini NX C18 5u 110A (100×30 mm), ACN gradient in water) to give 8-(2-chloro-5-methoxyphenyl)-N-(4-methanesulfonylpyridin-3-yl)quinoxalin-6-amine (10 mg; 0.02 mmol; yield: 15%; yellow powder; HPLC purity: >98%).

### Example 6, General Procedure 2

### N-(2-Methanesulfonylpyridin-3-yl)-8-(1-methyl-1H-indol-6-yl)quinoxalin-6-amine

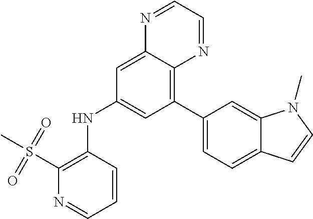 [SMILES: Cn1ccc2ccc(-c3cc(Nc4cccnc4S(C)(=O)=O)cc4nccnc34)cc21]

A pressure vessel is charged with 7-chloro-5-(1-methyl-1H-indol-6-yl)-quinoxaline (Intermediate 2B, 60 mg; 0.19 mmol; 1 eq.), 2-methanesulfonylpyridin-3-ylamine (65 mg; 0.38 mmol; 2 eq.), tBuONa (54 mg; 0.56 mmol; 3 eq.) and toluene (2.5 mL). The reaction mixture is flushed with argon before BINAP (23 mg; 0.04 mmol; 0.2 eq.) and Pd₂(dba)₃ (17 mg; 0.02 mmol; 0.10 eq.) are added. The tube is sealed and the reaction mixture is stirred for 20 h at 110° C. (oil bath temperature). The residue obtained after evaporation of the solvent is purified by FCC (0-5% MeOH gradient in DCM) to afford N-(2-methanesulfonylpyridin-3-yl)-8-(1-methyl-1H-indol-6-yl)quinoxalin-6-amine (57 mg; 0.13 mmol; yield: 69%; yellow solid; HPLC purity: 97.6%).

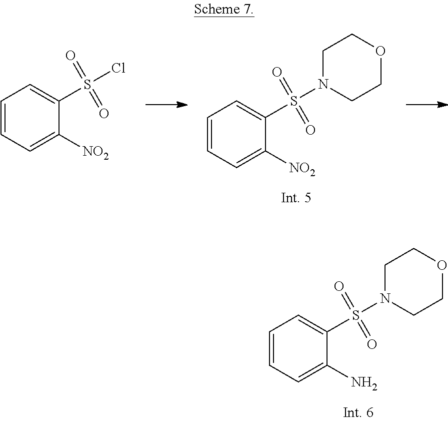 [SMILES: O=[N+]([O-])c1ccccc1S(=O)(=O)Cl ; O=[N+]([O-])c1ccccc1S(=O)(=O)N1CCOCC1 ; Nc1ccccc1S(=O)(=O)N1CCOCC1]

### Intermediate 5, General Procedure 3

### 4-(2-Nitrobenzenesulfonyl)morpholine

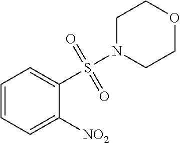 [SMILES: O=[N+]([O-])c1ccccc1S(=O)(=O)N1CCOCC1]

2-Nitrobenzenesulfonyl chloride (305 mg; 1.38 mmol; 1.2 eq.) is added to a stirred and pre-cooled (5° C.) suspension of NaHCO₃ (405 mg; 4.82 mmol; 4.2 eq) in water (0.13 mL) followed by morpholine (0.10 mL; 1.15 mmol; 1 eq.) and acetone (0.08 mL). The reaction mixture is stirred at ambient temperature for 2 h and diluted with water. Stirring is continued for an additional 20 min before extraction with EtOAc. The organic extracts are dried over Na₂SO₄, filtered and concentrated to provide 4-(2-nitro-benzenesulfonyl)morpholine (319 mg; 1.05 mmol; yield: 92%; white crystals; UPLC purity: 90%).

### Intermediate 6, General Procedure 4

### 2-(Morpholine-4-sulfonyl)aniline

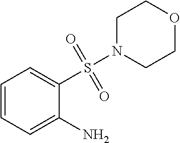 [SMILES: Nc1ccccc1S(=O)(=O)N1CCOCC1]

4-(2-Nitrobenzenesulfonyl)morpholine (Intermediate 5, 0.30 g; 0.99 mmol; 1 eq.) and palladium 10% on carbon (53 mg; 0.05 mmol; 0.05 eq.) are placed in a three-neck round-bottom flask equipped with an hydrogen balloon. Ethanol (5 mL) is added and the air is evacuated from the flask by applying vacuum and backfilling with hydrogen. The reaction is carried out under hydrogen atmosphere (1 atm) at room temperature overnight. The catalyst is filtered off through a pad of celite and the filter cake is washed with MeOH. The oily residue obtained by concentration of the filtrate is purified by FCC (EtOAc gradient in hexane) to afford 2-(morpholine-4-sulfonyl)aniline (254 mg; 1 mmol; yield: 100%; white solid; UPLC purity: 95%).

### Example 7

### 8-(1-Methyl-1H-indol-6-yl)-N-[2-(morpholine-4-sulfonyl)phenyl]quinoxalin-6-amine

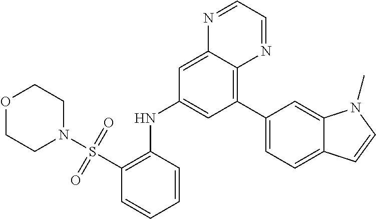 [SMILES: Cn1ccc2ccc(-c3cc(Nc4ccccc4S(=O)(=O)N4CCOCC4)cc4nccnc34)cc21]

The title compound is prepared according to General Procedure 1A described in Example 3, with 7-chloro-5-(1-methyl-1H-indol-6-yl)quinoxaline (Intermediate 2B, 50 mg; 0.16 mmol; 1 eq.), 2-(morpholine-4-sulfonyl)phenylamine (Intermediate 6, 99 mg; 0.39 mmol; 2.4 eq.), BINAP (20 mg; 0.03 mmol; 0.2 eq.), Pd₂(dba)₃ (38 mg; 0.02 mmol; 0.1 eq.) in toluene (4 mL). Conditions: 160° C. under microwave irradiation for 1 hour. Purification by FCC (EtOAc gradient in hexane) affords 8-(1-methyl-1H-indol-6-yl)-N-[2-(morpholine-4-sulfonyl)phenyl]quinoxalin-6-amine (65 mg; 0.13 mmol; yield: 79%; HPLC purity: 98%).

### Example 8, General Procedure 5

### 2-{[8-(1-Methyl-1H-indol-6-yl)quinoxalin-6-yl]amino}benzene-1-sulfonamide

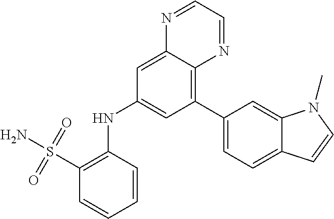 [SMILES: Cn1ccc2ccc(-c3cc(Nc4ccccc4S(N)(=O)=O)cc4nccnc34)cc21]

A pressure vessel is charged with 7-chloro-5-(1-methyl-1H-indol-6-yl)-quinoxaline (Intermediate 2B, 100 mg; 0.34 mmol; 1 eq.), 2-amino-benzenesulfonamide (70 mg; 0.41 mmol; 1.2 eq.), K₂CO₃ (94 mg; 0.68 mmol; 2 eq.), BippyPhos (34 mg; 0.07 mmol; 0.2 eq.) and dioxane (3 mL). The mixture is degassed by sonication and bubbling with argon before (Pd(cinnamyl)Cl)₂ (7 mg; 0.01 mmol; 0.04 eq.) is added. The reaction mixture is flushed with argon and stirred at 120° C. for 12 h. After cooling to room temperature, the reaction mixture is diluted with water and EtOAc. The aq. layer is extracted with EtOAc and the combined organic layers are washed with brine, dried over Na₂SO₄, filtered and concentrated. The residue is purified by FCC (EtOAc gradient in hexane; column neutralized with 1% Et₃N in DCM then washed with DCM before purification) to provide 2-{[8-(1-methyl-1H-indol-6-yl)quinoxalin-6-yl]amino}benzene-1-sulfonamide (18 mg; 0.04 mmol; yield: 11%; yellow powder; HPLC purity: 93.8%).

### Example 9

### 8-(1,3-Benzothiazol-5-yl)-N-(4-methanesulfonylpyridin-3-yl)quinoxalin-6-amine trifluoroacetate

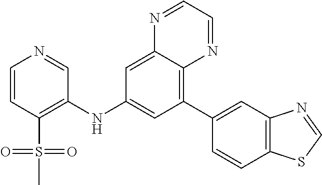 [SMILES: CS(=O)(=O)c1ccncc1Nc1cc(-c2ccc3scnc3c2)c2nccnc2c1]

A pressure vessel is loaded with 8-chloro-N-(4-methanesulfonylpyridin-3-yl)quinoxalin-6-amine (Intermediate 3B, 70 mg; 0.21 mmol; 1 eq.), 5-(4,4,5,5-tetramethyl-[1,3,2]dioxaborolan-2-yl)benzothiazole (109 mg; 0.42 mmol; 2 eq.), sodium carbonate (66 mg; 0.63 mmol; 3 eq.), DME (2 mL) and water (1 mL). The mixture is degassed by argon bubbling and sonication before Pd(dppf)Cl₂ (15 mg; 0.02 mmol; 0.10 eq.) is added. The tube is sealed and heated at 110° C. overnight. After cooling to room temperature the reaction mixture is filtered through a pad of celite and the filtrate is partitioned between EtOAc and water. The combined organic phases are washed with brine, dried over Na₂SO₄, filtered and concentrated. The residue is purified by FCC (0-5% MeOH gradient in DCM) and followed by reversed-phase preparative HPLC (column: Gemini NX C18 5u 110A (100×30 mm), ACN gradient in water) to give 8-(1,3-benzothiazol-5-yl)-N-(4-methanesulfonylpyridin-3-yl)quinoxalin-6-amine as its TFA salt (10 mg; 0.02 mmol; yield: 9%; orange powder; HPLC purity: >99%).

### Example 10, General Procedure 6

### N-(5-Bromo-2-methanesulfonylphenyl)-8-(1-methyl-1H-indol-6-yl)quinoxalin-6-amine

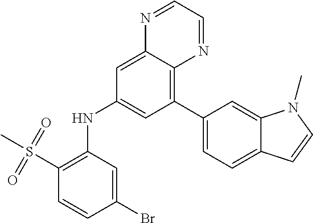 [SMILES: Cn1ccc2ccc(-c3cc(Nc4cc(Br)ccc4S(C)(=O)=O)cc4nccnc34)cc21]

8-(1-methyl-1H-indol-6-yl)-quinoxalin-6-ylamine (Intermediate 4, 50 mg; 0.17 mmol; 1 eq.) is dissolved in anhydrous DMF (0.50 mL) and NaH (60% in mineral oil, 9 mg; 0.36 mmol; 2.3 eq.) is added in one portion. The reaction mixture is stirred at room temperature for 30 min before 4-bromo-2-fluoro-1-methanesulfonylbenzene (55 mg; 0.36 mmol; 2.30 eq.) is added. Stirring is continued at 90° C. overnight. The reaction mixture is partitioned between DCM and water and the aq. phase is extracted with DCM. The combined organic layers are washed with brine, dried over Na₂SO₄ and concentrated. The crude product is purified by FCC (0-10% EtOAc gradient in hexane) and re-purified by reversed-phase preparative HPLC (column: Gemini NX C18 5u 110A (100×30 mm), ACN gradient in water) to afford N-(5-bromo-2-methanesulfonylphenyl)-8-(1-methyl-1H-indol-6-yl)quinoxalin-6-amine (11 mg; 0.02 mmol; yield: 13%; yellow powder; HPLC purity: 99.4%).

### Example 11

### N-(4-Methanesulfonylpyridin-3-yl)-8-(1-methyl-1H-indol-6-yl)quinoxalin-6-amine

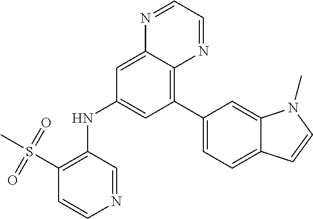 [SMILES: Cn1ccc2ccc(-c3cc(Nc4cnccc4S(C)(=O)=O)cc4nccnc34)cc21]

The title compound is prepared according to General Procedure 2 described in Example 6, with 7-chloro-5-(1-methyl-1H-indol-6-yl)quinoxaline (Intermediate 2B, 50 mg; 0.17 mmol; 1 eq.), 4-methanesulfonylpyridin-3-ylamine hydrochloride (43 mg; 0.20 mmol; 1.2 eq.), tBuONa (49 mg; 0.51 mmol; 3 eq.), BINAP (11 mg; 0.02 mmol; 0.1 eq.), Pd₂(dba)₃ (8 mg; 0.01 mmol; 0.05 eq.) in toluene (2 mL). Purification by FCC (0-100% EtOAc gradient in hexane continued with 0-5% MeOH gradient in EtOAc) provided N-(4-methanesulfonylpyridin-3-yl)-8-(1-methyl-1H-indol-6-yl)quinoxalin-6-amine (50 mg; 0.12 mmol; yield: 68%; yellow powder; HPLC purity: 99.3%).

### Example 13

### N-(2-Methoxypyridin-3-yl)-8-(1-methyl-1H-indol-6-yl)quinoxalin-6-amine

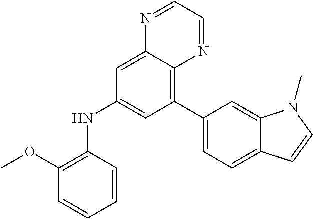 [SMILES: COc1ccccc1Nc1cc(-c2ccc3ccn(C)c3c2)c2nccnc2c1]

The title compound is prepared according to General Procedure 1A described in Example 3 with 7-chloro-5-(1-methyl-1H-indol-6-yl)quinoxaline (Intermediate 2B, 100 mg; 0.34 mmol; 1 eq.), 2-methoxy-pyridin-3-ylamine (47 μL; 0.60 mmol; 1.8 eq.), tBuONa (65 mg; 0.67 mmol; 2 eq.), BINAP (42 mg; 0.1 mmol; 0.30 eq.), Pd₂(dba)_{3 (}31 mg; 0.05 mmol; 0.15 eq.) in toluene (3 mL). Conditions: 100° C. under conventional heating for 6 hours. Purification by FCC (0-5% MeOH gradient in DCM) affords N-(2-methoxypyridin-3-yl)-8-(1-methyl-1H-indol-6-yl)quinoxalin-6-amine (67 mg; 0.17 mmol; yield: 52%; yellow solid; HPLC purity: 99.7%).

### Example 14, General Procedure 7

### 3-{[8-(1-Methyl-1H-indol-6-yl)quinoxalin-6-yl]amino}pyridin-2-ol

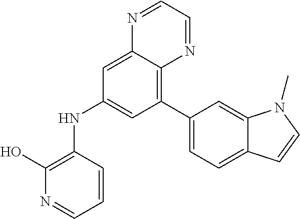 [SMILES: Cn1ccc2ccc(-c3cc(Nc4cccnc4O)cc4nccnc34)cc21]

A pressure vessel is charged with 7-chloro-5-(1-methyl-1H-indol-6-yl)-quinoxaline (Intermediate 2B, 60 mg; 0.20 mmol; 1 eq.), 3-amino-1H-pyridin-2-one (27 mg; 0.24 mmol; 1.2 eq.), BrettPhos (8 mg; 0.01 mmol; 0.07 eq.) and BrettPhos precatalyst (11 mg; 0.01 mmol; 0.07 eq.). The tube is flushed with argon and LiHMDS (1 M in THF, 0.49 mL; 0.49 mmol; 2.40 eq.) is added by syringe. The reaction mixture is stirred at 65° C. for 1.5 h. MeOH is added and stirring is continued for 5 minutes. The reaction mixture is concentrated and the residue is purified by FCC (0-5% MeOH gradient in DCM) to afford 3-{[8-(1-methyl-1H-indol-6-yl)quinoxalin-6-yl]amino}pyridin-2-ol (25 mg; 0.07 mmol; yield: 32%; green solid; HPLC purity: 96.5%).

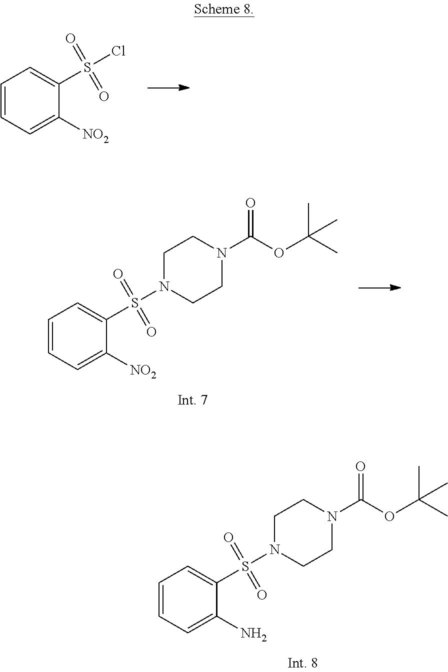 [SMILES: O=[N+]([O-])c1ccccc1S(=O)(=O)Cl ; CC(C)(C)OC(=O)N1CCN(S(=O)(=O)c2ccccc2[N+](=O)[O-])CC1 ; CC(C)(C)OC(=O)N1CCN(S(=O)(=O)c2ccccc2N)CC1]

### Intermediate 7

### tert-Butyl 4-(2-nitrobenzenesulfonyl)piperazine-1-carboxylate

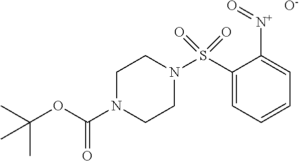

The title compound is prepared according to General Procedure 3 described for Intermediate 5, with 2-nitrobenzenesulfonyl chloride (286 mg; 1.29 mmol; 1.2 eq.), NaHCO₃ (379 mg; 4.51 mmol; 4.2 eq.), piperazine-1-carboxylic acid tert-butyl ester (0.20 mL; 1.07 mmol; 1 eq.) in water (1 mL) and acetone (0.15 mL) for 2 hours at room temperature to afford tert-butyl 4-(2-nitrobenzenesulfonyl)-piperazine-1-carboxylate (0.46 g; 0.83 mmol; beige oil; yield: 77.1%; UPLC purity: 67%).

### Intermediate 8

### tert-Butyl 4-(2-aminobenzenesulfonyl)piperazine-1-carboxylate

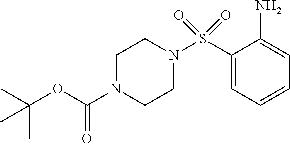 [SMILES: CC(C)(C)OC(=O)N1CCN(S(=O)(=O)c2ccccc2N)CC1]

The title compound is prepared according to General Procedure 4 (synthesis of Intermediate 6) with tert-butyl 4-(2-nitrobenzenesulfonyl)piperazine-1-carboxylate (0.40 g; 0.72 mmol; 1 eq.), palladium 10% on carbon (38 mg; 0.04 mmol; 0.05 eq.) in EtOH (5 mL) for 18 hours at room temperature. Purification by FCC (EtOAc gradient in hexane) affords tert-butyl 4-(2-aminobenzenesulfonyl)-piperazine-1-carboxylate (183 mg; 0.35 mmol; yield: 50%; UPLC purity: 58%).

### Intermediate 9

### tert-Butyl 4-(2-{[8-(1-methyl-1H-indol-6-yl)quinoxalin-6-yl]amino}-benzenesulfonyl)piperazine-1-carboxylate

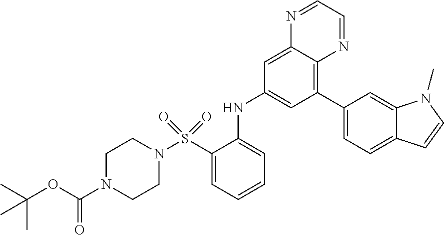 [SMILES: Cn1ccc2ccc(-c3cc(Nc4ccccc4S(=O)(=O)N4CCN(C(=O)OC(C)(C)C)CC4)cc4nccnc34)cc21]

The title compound is prepared according to General Procedure 1A described in Example 3 with 7-chloro-5-(1-methyl-1H-indol-6-yl)quinoxaline (Intermediate 2B, 47 mg; 0.16 mmol; 1 eq.), tert-butyl 4-(2-aminobenzenesulfonyl)piperazine-1-carboxylate (Intermediate 8, 140 mg; 0.24 mmol; 1.5 eq.), BINAP (20 mg; 0.03 mmol; 0.20 eq.), Pd₂(dba)₃ (37 mg; 0.02 mmol; 0.1 eq.) in toluene (4 mL) at 160° C. under microwave irradiation for 1 hour. Purification by FCC (EtOAc gradient in hexane) provided tert-butyl 4-(2-{[8-(1-methyl-1H-indol-6-yl)quinoxalin-6-yl]amino}benzenesulfonyl)piperazine-1-carboxylate (39 mg; 0.05 mmol; yield: 32%; brown oil; UPLC purity: 78%).

### Example 15

### 8-(1-Methyl-1H-indol-6-yl)-N-[2-(piperazine-1-sulfonyl)phenyl]quinoxalin-6-amine trifluoroacetate

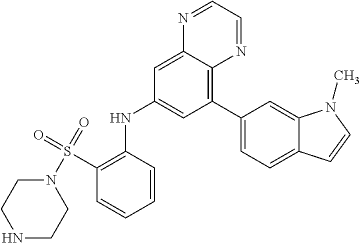 [SMILES: Cn1ccc2ccc(-c3cc(Nc4ccccc4S(=O)(=O)N4CCNCC4)cc4nccnc34)cc21]

4-{2-[8-(1-Methyl-1H-indol-6-yl)-quinoxalin-6-ylamino]-benzenesulfonyl}-piperazine-1-carboxylic acid tert-butyl ester (Intermediate 9, 39 mg; 0.05 mmol; 1 eq.) is dissolved in DCM (3 mL) and treated with TFA (116 mg; 1.02 mmol; 20 eq.). The reaction mixture is stirred overnight at room temperature. The volatiles are evaporated under reduced pressure and the brown residue is purified by FCC (EtOAc gradient in hexane) and re-purified by reversed-phase preparative HPLC (column: Gemini NX C18 5u 110A (100×30 mm), ACN gradient in water) to afford 8-(1-methyl-1H-indol-6-yl)-N-[2-(piperazine-1-sulfonyl)phenyl]quinoxalin-6-amine trifluoroacetate (8 mg; 0.01 mmol; yield: 23%; beige solid; HPLC purity: 89%).

### Example 16

### N-Methyl-2-{[8-(1-methyl-1H-indol-6-yl)quinoxalin-6-yl]amino}benzene-1-sulfonamide

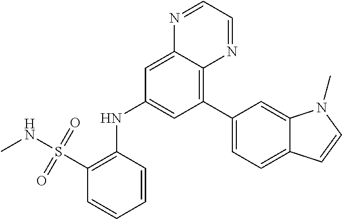 [SMILES: CNS(=O)(=O)c1ccccc1Nc1cc(-c2ccc3ccn(C)c3c2)c2nccnc2c1]

The title compound is prepared according to General Procedure 1A described in Example 3 with 7-chloro-5-(1-methyl-1H-indol-6-yl)quinoxaline (Intermediate 2B, 60 mg; 0.19 mmol; 1 eq.), 2-amino-N-methyl-benzenesulfonamide (87 mg; 0.47 mmol; 2.4 eq.), tBuONa (56 mg; 0.58 mmol; 3 eq.), BINAP (24 mg; 0.04 mmol; 0.2 eq.), Pd₂(dba)₃ (45 mg; 0.02 mmol; 0.1 eq.) in toluene (4 mL) at 160° C. under microwave irradiation for 1 hour. Purification by FCC (EtOAc gradient in hexane, column neutralized with 1% Et₃N in DCM and washed with DCM beforehand) affords N-methyl-2-{[8-(1-methyl-1H-indol-6-yl)quinoxalin-6-yl]amino}benzene-1-sulfonamide (53 mg; 0.11 mmol; yield: 55%; yellow amorphous powder; HPLC purity: 90%).

### Intermediate 10

### 8-(1-Methyl-1H-indol-6-yl)-N-(2-nitropyridin-3-yl)quinoxalin-6-amine

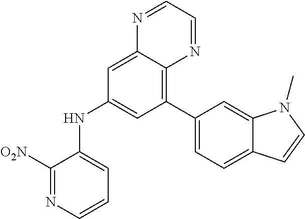 [SMILES: Cn1ccc2ccc(-c3cc(Nc4cccnc4[N+](=O)[O-])cc4nccnc34)cc21]

The title compound is prepared according to General Procedure 5 described in Example 8 with 7-chloro-5-(1-methyl-1H-indol-6-yl)quinoxaline (Intermediate 2B, 60 mg; 0.20 mmol; 1 eq.), 2-nitro-pyridin-3-ylamine (34 mg; 0.25 mmol; 1.2 eq.), K₂CO₃ (56 mg; 0.41 mmol; 2 eq.), BippyPhos (21 mg; 0.04 mmol; 0.2 eq.), (Pd(cinnamyl)Cl)₂ (4 mg; 0.01 mmol; 0.04 eq.) in dioxane (3 mL) at 120° C. under conventional heating for 12 hours. Purification by FCC (EtOAc gradient in hexane, column neutralized with 1% Et₃N in DCM and washed with DCM beforehand) provided 8-(1-methyl-1H-indol-6-yl)-N-(2-nitropyridin-3-yl)quinoxalin-6-amine (62 mg; 0.09 mmol; yield: 52%; yellow powder; HPLC purity: 73%).

### Example 17

### 3-N-[8-(1-methyl-1H-indol-6-yl)quinoxalin-6-yl]pyridine-2,3-diamine

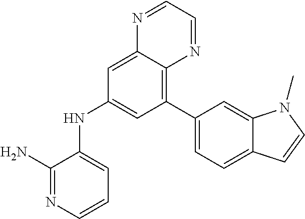 [SMILES: Cn1ccc2ccc(-c3cc(Nc4cccnc4N)cc4nccnc34)cc21]

8-(1-Methyl-1H-indol-6-yl)-N-(2-nitropyridin-3-yl)quinoxalin-6-amine (Intermediate 10, 37 mg; 0.07 mmol; 1 eq.) is dissolved in EtOAc (3 mL), then 10% palladium on carbon (10 mg; 0.01 mmol; 0.14 eq.) is added. The flask is equipped with a hydrogen balloon and the reaction mixture is stirred under hydrogen atmosphere at room temperature for 1 h, at which point TLC showed completion of the reaction. The reaction mixture is filtered off through a pad of celite, and the filter cake is washed with EtOAc. The filtrate is concentrated and the residue is purified by FCC (MeOH gradient in DCM) to afford 3-N-[8-(1-methyl-1H-indol-6-yl)quinoxalin-6-yl]pyridine-2,3-diamine (7 mg; 0.02 mmol; yield: 28%; yellow powder; HPLC purity: 98%).

### Example 18

### 3-{[8-(1-Methyl-1H-indol-6-yl)quinoxalin-6-yl]amino}pyridine-4-carbonitrile

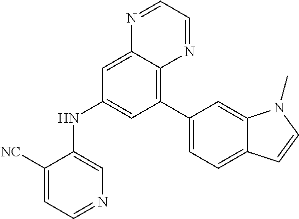 [SMILES: Cn1ccc2ccc(-c3cc(Nc4cnccc4C#N)cc4nccnc34)cc21]

The title compound is prepared according to General Procedure 5 described in Example 8 with 7-chloro-5-(1-methyl-1H-indol-6-yl)quinoxaline (Intermediate 2B, 60 mg; 0.20 mmol; 1 eq.), 3-amino-isonicotinonitrile (29 mg; 0.25 mmol; 1.2 eq.), K₂CO₃ (56 mg; 0.41 mmol; 2 eq.), BippyPhos (21 mg; 0.04 mmol; 0.2 eq.), (Pd(cinnamyl)Cl)₂ (4 mg; 0.01 mmol; 0.04 eq.) in dioxane (3 mL). Conditions: 120° C. for 12 h. Purification by FCC (MeOH gradient in DCM, column neutralized with 1% Et₃N in DCM and washed with DCM beforehand) affords 3-{[8-(1-methyl-1H-indol-6-yl)quinoxalin-6-yl]amino}pyridine-4-carbonitrile (35 mg; 0.09 mmol; yield: 44.9%; yellow powder; HPLC purity: 97%).

### Example 19

### 3-{[8-(1-Methyl-1H-indol-6-yl)quinoxalin-6-yl]amino}pyridine-4-carboxamide

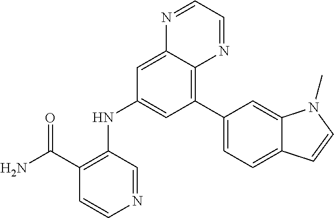 [SMILES: Cn1ccc2ccc(-c3cc(Nc4cnccc4C(N)=O)cc4nccnc34)cc21]

3-{[8-(1-methyl-1H-indol-6-yl)quinoxalin-6-yl]amino}pyridine-4-carbonitrile (Example 18, 15 mg; 0.04 mmol; 1 eq.) is dissolved in a suspension of KOH (7 mg; 0.12 mmol; 3 eq.) in tBuOH (2 mL). The reaction mixture is stirred at 60° C. under argon for 5 h, at which point TLC showed completion of the reaction. Water is added to the reaction mixture and the product is extracted with EtOAc. The organic phase is washed with brine, dried over Na₂SO₄, filtered and concentrated in vacuo to provide 3-{[8-(1-methyl-1H-indol-6-yl)quinoxalin-6-yl]amino}pyridine-4-carboxamide (8 mg; 0.02 mmol; yield: 48%; yellow powder; HPLC purity: >92%).

### Example 20

### N,N-Dimethyl-2-{[8-(1-methyl-1H-indol-6-yl)quinoxalin-6-yl]amino}benzene-1-sulfonamide

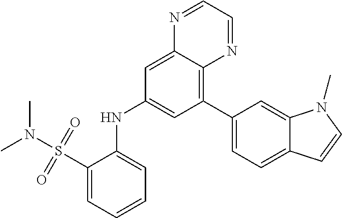 [SMILES: CN(C)S(=O)(=O)c1ccccc1Nc1cc(-c2ccc3ccn(C)c3c2)c2nccnc2c1]

The title compound is prepared according to General Procedure 2 described in Example 6 with 7-Chloro-5-(1-methyl-1H-indol-6-yl)quinoxaline (Intermediate 2B, 60 mg; 0.19 mmol; 1 eq.), 2-amino-N,N-dimethyl-benzenesulfonamide (93 mg; 0.47 mmol; 2.4 eq.), tBuONa (56 mg; 0.58 mmol; 3 eq.), BINAP (24 mg; 0.04 mmol; 0.2 eq.), Pd₂(dba)₃ (45 mg; 0.02 mmol; 0.1 eq.) in toluene (4 mL). Conditions: 160° C. under microwave irradiation for 1 hour. Purification by FCC (EtOAc gradient in hexane) affords N,N-dimethyl-2-{[8-(1-methyl-1H-indol-6-yl)quinoxalin-6-yl]amino}benzene-1-sulfonamide (81 mg; 0.18 mmol; yield: 91%; yellow amorphous powder; HPLC purity: 100%).

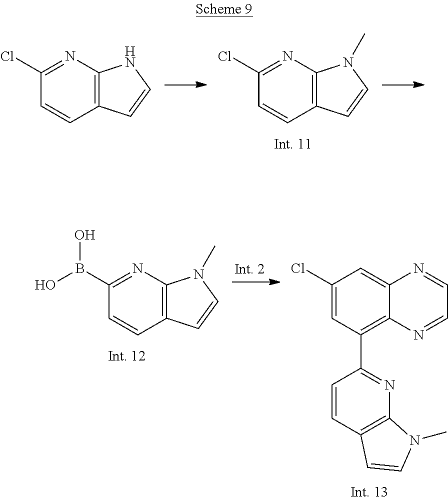 [SMILES: Clc1ccc2cc[nH]c2n1 ; Cn1ccc2ccc(Cl)nc21 ; Cn1ccc2ccc(B(O)O)nc21 ; Cn1ccc2ccc(-c3cc(Cl)cc4nccnc34)nc21]

### Intermediate 11

### 6-Chloro-1-methyl-1H-pyrrolo[2,3-b]pyridine

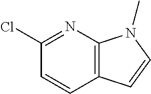

To a stirred solution of 6-chloro-1H-pyrrolo[2,3-b]pyridine (500 mg; 3.28 mmol; 1 eq.) in DMF, sodium hydride solution (60% in mineral oil, 157 mg; 3.93 mmol; 1.2 eq.) is added portionwise at 0-5° C. After 30 minutes iodomethane (0.14 mL; 2.29 mmol; 0.7 eq.) is added dropwise. Stirring is continued for 30 min at 5° C. and at room temperature for 1 h. The reaction is quenched with water and extracted with EtOAc. The combined organic phases are washed with water, dried over Na₂SO₄, filtered and concentrated. The residue is purified by FCC (EtOAc gradient in hexane, column neutralized with 1% Et₃N in DCM and washed DCM beforehand) to afford 6-chloro-1-methyl-1H-pyrrolo[2,3-b]pyridine (499 mg; 2.78 mmol; yield 85%; colorless liquid; UPLC purity 92.9%).

### Intermediate 12

### (1-Methylpyrrolo[2,3-b]pyridin-6-yl)boronic acid

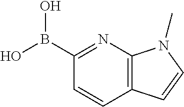

A pressure vessel is charged with 6-chloro-1-methyl-1H-pyrrolo[2,3-b]pyridine (Intermediate 11 (499 mg; 2.78 mmol; 1 eq.) and 4,4,5,5,4′,4′,5′,5′-octamethyl-[2,2′]bis[[1,3,2]dioxaborolanyl] (848 mg; 3.34 mmol; 1.2 eq.) and dioxane (12 mL) under argon. The mixture is sonicated under a stream of argon before potassium acetate (1.36 g; 13.91 mmol; 5 eq.) and Pd(dppf)Cl₂CH₂Cl₂ (227 mg; 0.28 mmol; 0.1 eq.) are added. The tube is sealed and the reaction mixture is stirred at 100° C. (oil bath temperature) for 5 hours. The reaction mixture is concentrated and the residue is dissolved in n-butanol, washed with water (three times), dried over Na₂SO₄, filtered and concentrated to give crude 1-methylpyrrolo[2,3-b]pyridin-6-yl)boronic acid (815 mg) used in the consecutive step without further purification.

### Intermediate 13

### 7-Chloro-5-(1-methyl-1H-pyrrolo[2,3-b]pyridin-6-yl)quinoxaline

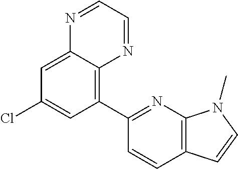 [SMILES: Cn1ccc2ccc(-c3cc(Cl)cc4nccnc34)nc21]

A pressure vessel is loaded with 5-bromo-7-chloro-quinoxaline (Intermediate 2, 80 mg; 0.33 mmol; 1 eq.), (1-methylpyrrolo[2,3-b]pyridin-6-yl)boronic acid (Intermediate 12, 99 mg; 0.39 mmol; 1.2 eq.), 2 M aq. sodium carbonate (0.33 mL; 0.66 mmol; 2 eq.), dioxane (2 mL) and water (1 mL). The reaction mixture is sparged with argon before Pd(PPh₃)₄ (38 mg; 0.03 mmol; 0.1 eq.) is added. The reaction tube is sealed and the reaction mixture is stirred at 100° C. for 4 h. The reaction mixture is diluted with EtOAc and filtered through a pad of celite and the filtrate is concentrated in vacuo. The residue is purified by FCC (EtOAc gradient in hexane, column neutralized with 1% Et₃N in DCM and washed with DCM beforehand) to afford 7-chloro-5-(1-methyl-1H-pyrrolo[2,3-b]pyridin-6-yl)-quinoxaline (25 mg; 0.08 mmol; yield: 24%; white powder; UPLC purity: 92%).

### Example 21

### N-(2-Methanesulfonylphenyl)-8-{1-methyl-1H-pyrrolo[2,3-b]pyridin-6-yl}quinoxalin-6-amine trifluoroacetate

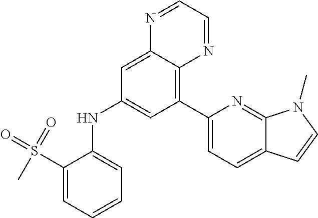 [SMILES: Cn1ccc2ccc(-c3cc(Nc4ccccc4S(C)(=O)=O)cc4nccnc34)nc21]

The title compound is prepared according to General Procedure 1A with 7-chloro-5-(1-methyl-1H-pyrrolo[2,3-b]pyridin-6-yl)quinoxaline (Intermediate 13, 25 mg; 0.08 mmol; 1 eq.), 2-methanesulfonylphenylamine hydrochloride (39 mg; 0.19 mmol; 2.40 eq.), tBuONa (30 mg; 0.32 mmol; 4 eq.), BINAP (10 mg; 0.02 mmol; 0.2 eq.), Pd₂(dba)₃ (18 mg; 0.01 mmol; 0.1 eq.) in toluene (2 mL). Conditions: 160° C. under microwave irradiation for 1 hour. Purification by FCC (MeOH gradient in DCM) and reversed-phase preparative HPLC (column: Gemini NX C18 5u 110A (100×30 mm), ACN gradient in water) affords (2-methanesulfonyl-phenyl)-[8-(1-methyl-1H-pyrrolo[2,3-b]pyridin-6-yl)-quinoxalin-6-yl]-amine trifluoroacetate (5 mg; 0.01 mmol; 12%; red powder; HPLC purity: 100%).

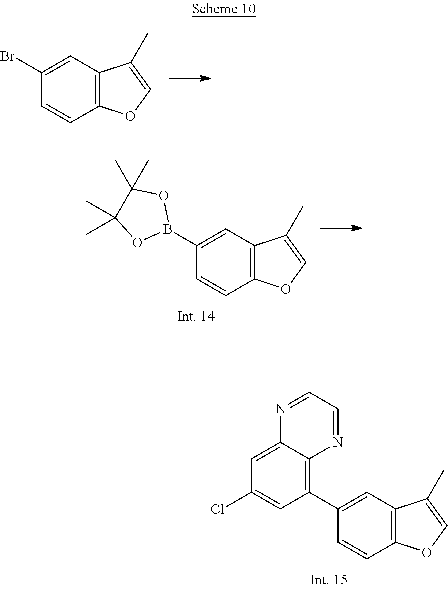

### Intermediate 14, General Procedure 8

### 3-Methyl-5-(4,4,5,5-tetramethyl-[1,3,2]dioxaborolan-2-yl)benzofuran

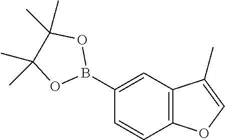 [SMILES: Cc1coc2ccc(B3OC(C)(C)C(C)(C)O3)cc12]

A pressure vessel is charged with a solution of 5-bromo-3-methylbenzofuran (467 mg; 2.21 mmol; 1 eq.) in dioxane (6 mL) under argon. To this solution, KOAc (543 mg; 5.53 mmol; 2.50 eq.) and bis(pinacolato)diboron (674 mg; 2.66 mmol; 1.2 eq.) are added under argon. The solution is additionally sparged with argon with sonication and Pd(dppf)Cl₂ (81 mg; 0.11 mmol; 0.05 eq.) is added. The reaction mixture is stirred for 14 hours at 95° C. then cooled to room temperature and partitioned between water and EtOAc. The aqueous layer is extracted with EtOAc and the combined organic phases are washed with saturated aq. NaHCO₃ and brine, dried over anhydrous sodium sulfate, filtered and concentrated. The residue is purified by FCC (0-10% EtOAc gradient in hexane) to afford 3-methyl-5-(4,4,5,5-tetramethyl-[1,3,2]dioxaborolan-2-yl)benzofuran (377 mg; 1.43 mmol; yield: 65%; brown solid; HPLC purity: 98%).

### Intermediate 15 General Procedure 9

### 7-Chloro-5-(3-methyl-1-benzofuran-5-yl)quinoxaline

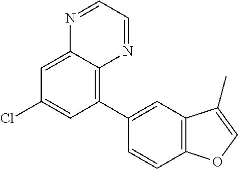 [SMILES: Cc1coc2ccc(-c3cc(Cl)cc4nccnc34)cc12]

A pressure vessel is charged with 5-bromo-7-chloro-quinoxaline (Intermediate 2, 300 mg; 1.18 mmol; 1 eq.), 3-methyl-5-(4,4,5,5-tetramethyl-[1,3,2]dioxaborolan-2-yl)-benzofuran (Intermediate 14, 311 mg; 1.18 mmol; 1 eq.), cesium carbonate (771 mg; 2.37 mmol; 2 eq.), 1,2-dimethoxyethane (15 mL) and water (5 mL). The reaction mixture is sparged with argon under sonication before Pd(dppf)Cl₂CH₂Cl₂ (145 mg; 0.18 mmol; 0.15 eq.) is added. The tube is sealed and the reaction mixture is stirred at 100° C. for 1 h at which point UPLC analysis showed complete conversion. The reaction mixture is cooled to room temperature, diluted with EtOAc and washed with water and brine. The organic layer is dried over Na₂SO₄ and filtered through a pad of celite. The filtrate is concentrated in vacuo and the resulting residue is purified by FCC (20-75% EtOAc gradient in hexane) to give 7-chloro-5-(3-methyl-1-benzofuran-5-yl)-quinoxaline (234 mg; 0.79 mmol; yield: 66%; white powder; HPLC purity: 99%).

### Example 22

### N-(4-Methanesulfonylpyridin-3-yl)-8-(3-methyl-1-benzofuran-5-yl)quinoxalin-6-amine

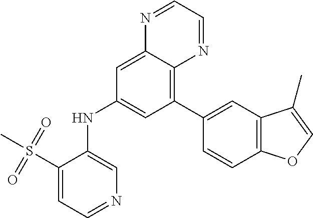 [SMILES: Cc1coc2ccc(-c3cc(Nc4cnccc4S(C)(=O)=O)cc4nccnc34)cc12]

The title compound is prepared according to General Procedure 2 described in Example 6 with 7-chloro-5-(3-methyl-benzofuran-5-yl)-quinoxaline (Intermediate 15, 65 mg; 0.22 mmol; 1 eq.), 4-methanesulfonylpyridin-3-ylamine hydrochloride (55 mg; 0.26 mmol; 1.2 eq.), tBuOK (61 mg; 0.53 mmol; 2.4 eq., tBuONa in original procedure), BINAP (14 mg; 0.02 mmol; 0.1 eq.) and Pd₂(dba)₃ (10 mg; 0.01 mmol; 0.05 eq.) in toluene (2 mL). After 16 h at 130° C., UPLC-MS analysis showed 28% of conversion. Another portion of tBuOK (12 mg; 0.11 mmol; 0.5 eq.) and Pd₂(dba)₃ (10 mg; 0.01 mmol; 0.05 eq.) are added under argon atmosphere. The reaction mixture is stirred at 130° C. for 10 h, diluted with EtOAc, washed with water and brine, then dried over Na₂SO₄ and filtered through a pad of Celite. The filtrate is concentrated in vacuo and the crude product is purified by FCC (10-100% EtOAc gradient in hexane) to afford (4-methanesulfonylpyridin-3-yl)-[8-(3-methyl-benzofuran-5-yl)-quinoxalin-6-yl]-amine (37 mg; 0.08 mmol; yield 36%; pale yellow powder; HPLC purity 93%).

### Example 23

### N-(4-methoxypyridin-3-yl)-8-(1-methyl-1H-indol-6-yl)quinoxalin-6-amine

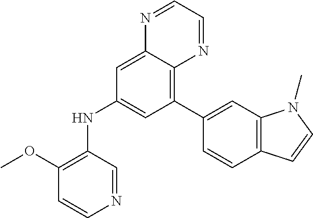 [SMILES: COc1ccncc1Nc1cc(-c2ccc3ccn(C)c3c2)c2nccnc2c1]

The title compound is prepared according to General Procedure 2 described in Example 6 with 7-chloro-5-(1-methyl-1H-indol-6-yl)quinoxaline (60 mg; 0.19 mmol; 1 eq.), 4-methoxypyridin-3-ylamine (60 mg; 0.46 mmol; 2.4 eq.), tBuONa (55 mg; 0.58 mmol; 3 eq.), Pd₂(dba)₃ (18 mg; 0.02 mmol; 0.1 eq.) and BINAP (25 mg; 0.04 mmol; 0.2 eq.) in toluene. Conditions: 110° C., 16 h. Purification by FCC (10-100% EtOAc gradient in hexane) followed by reversed-phase preparative HPLC (column: Gemini NX C18 5u 110A (100×30 mm), ACN gradient in water) affords N-(4-methoxypyridin-3-yl)-[8-(1-methyl-1H-indol-6-yl)quinoxalin-6-yl]amine (20 mg; 0.05 mmol; yield: 27%; HPLC purity 99%).

### Example 24

### 3-{[8-(3-methyl-1-benzofuran-5-yl)quinoxalin-6-yl]amino}pyridine-4-carbonitrile

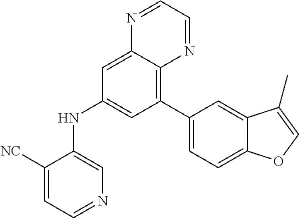 [SMILES: Cc1coc2ccc(-c3cc(Nc4cnccc4C#N)cc4nccnc34)cc12]

The title compound is prepared according to General Procedure 5 described in example 8 with 7-chloro-5-(3-methyl-benzofuran-5-yl)-quinoxaline (50 mg; 0.17 mmol; 1 eq.), 3-amino-isonicotinonitrile (24 mg; 0.20 mmol; 1.2 eq.), K₂CO₃ (47 mg; 0.34 mmol; 2 eq.), BippyPhos (17 mg; 0.03 mmol; 0.2 eq.) and (Pd(cinnamyl)Cl)₂ (4 mg; 0.01 mmol; 0.04 eq.) in dioxane (3 mL). Conditions: 120° C. 12 hours. Purification by FCC (EtOAc gradient in hexane) affords 3-{[8-(3-methyl-1-benzofuran-5-yl)quinoxalin-6-yl]amino}pyridine-4-carbonitrile (13 mg; 0.03 mmol; yield 20%; yellow powder; HPLC purity: 99.7%).

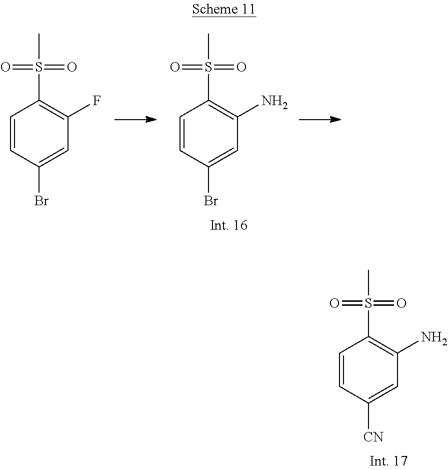

### Intermediate 16

### 5-Bromo-2-methanesulfonylphenylamine

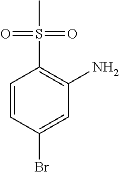

A pressure vessel is charged with 4-bromo-2-fluoro-1-methanesulfonyl-benzene (1 g; 3.75 mmol; 1 eq.) and DMSO (20 mL) followed by aqueous ammonia (25%, 12 mL; 75 mmol; 20 eq.) and the reaction mixture is stirred at 130° C. overnight. After coming back to room temperature, the reaction mixture is poured into water (100 mL) and is vigorously stirred for 30 minutes. The precipitating white solid is filtered, washed with water and dried in vacuo to yield 5-bromo-2-methanesulfonylphenylamine (795 mg; 3.15 mmol; yield: 84%; white powder; UPLC purity 99.5%).

### Intermediate 17

### 3-Amino-4-methanesulfonylbenzonitrile

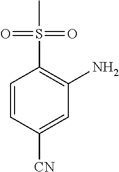

A pressure vessel is charged with 5-bromo-2-methanesulfonyl phenylamine (Intermediate 16, 50 mg; 0.20 mmol; 1 eq.), tetrapotassium hexacyanoiron trihydrate (34 mg; 0.08 mmol; 0.4 eq., freshly grinded), DBU (7 μL; 0.05 mmol; 0.25 eq.), tert-butanol (0.50 mL) and water (0.50 mL). The reaction mixture is bubbled with argon for 5 minutes and Pd(PPh₃)₄ (23 mg; 0.02 mmol; 0.1 eq.) is added. The reaction tube is sealed and the reaction mixture is stirred at 85° C. for 6 hours, at which point TLC showed completion of the reaction. The reaction mixture is diluted with DCM and filtered through a pad of Celite. Water is added to the filtrate and the phases are separated. The aqueous layer is extracted three times with DCM and the combined organic phases are washed with brine, dried over Na₂SO₄ and concentrated in vacuo. The residue is dissolved in DCM and purified by FCC (0-30% EtOAc gradient in hexane) to afford 3-amino-4-methanesulfonyl-benzonitrile (47 mg; 0.24 mmol; 60%; beige powder; UPLC purity 100%).

### Example 25

### 4-Methanesulfonyl-3-{[8-(1-methyl-1H-indol-6-yl)quinoxalin-6-yl]amino}benzonitrile

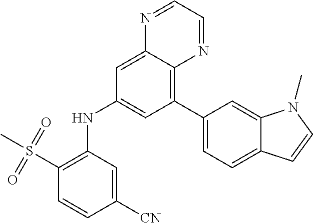 [SMILES: Cn1ccc2ccc(-c3cc(Nc4cc(C#N)ccc4S(C)(=O)=O)cc4nccnc34)cc21]

The title compound is prepared according to General Procedure 5 described in example 8, with 7-chloro-5-(1-methyl-1H-indol-6-yl)quinoxaline (Intermediate 2B, 60 mg; 0.19 mmol; 1 eq.), 3-amino-4-methanesulfonyl-benzonitrile (Intermediate 17, 46 mg; 0.23 mmol; 1.2 eq.), tBuONa (26 mg; 0.27 mmol; 1.4 eq.), BippyPhos (20 mg; 0.04 mmol; 0.20 eq.) and (Pd(cinnamyl)Cl)₂ (5 mg; 0.01 mmol; 0.05 eq.) in toluene (2 mL). Conditions: and 100° C. for 16 hours. Purification by FCC (EtOAc gradient in hexane) and by reversed-phase preparative HPLC (column: Gemini NX C18 5u 110A (100×30 mm), ACN gradient in water) affords 4-methanesulfonyl-3-{[8-(1-methyl-1H-indol-6-yl)quinoxalin-6-yl]amino}benzonitrile (15 mg; 0.03 mmol; yield: 17%; pale yellow powder; HPLC purity 99.8%).

### Example 26

### 3-{[8-(3-Methyl-1-benzofuran-5-yl)quinoxalin-6-yl]amino}pyridine-4-carboxamide

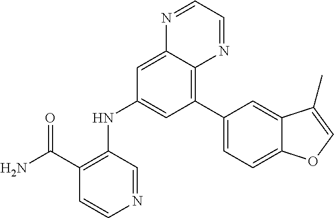 [SMILES: Cc1coc2ccc(-c3cc(Nc4cnccc4C(N)=O)cc4nccnc34)cc12]

The title compound is prepared according to General Procedure 5 described in Example 8 with 7-chloro-5-(3-methyl-benzofuran-5-yl)-quinoxaline (Intermediate 15, 50 mg; 0.17 mmol; 1 eq.), 3-aminoisonicotinamide (28 mg; 0.20 mmol; 1.2 eq.), K₂CO₃ (47 mg; 0.34 mmol; 2 eq.), BippyPhos (17 mg; 0.03 mmol; 0.2 eq.), (Pd(cinnamyl)Cl)₂ (4 mg; 0.01 mmol; 0.04 eq.) in dioxane (3 mL). Conditions: 120° C., 6 h. Purification by FCC (0-5% MeOH gradient in DCM) followed by reversed-phase preparative HPLC (column: Gemini NX C18 5u 110A (100×30 mm), ACN gradient in water) affords 3-{[8-(3-methyl-1-benzofuran-5-yl)quinoxalin-6-yl]amino}pyridine-4-carboxamide (3 mg; 0.01 mmol; yield: 5%; yellow powder; HPLC purity 98.9%).

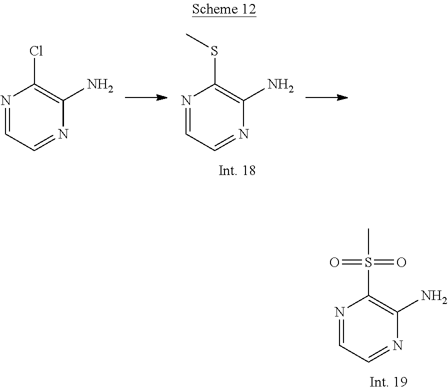

### Intermediate 18, General Procedure 10

### 3-Methylsulfanylpyrazin-2-ylamine

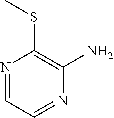

To a solution of 3-chloropyrazin-2-ylamine (250 mg; 1.93 mmol; 1 eq.) in DMF (2 mL) and EtOH (2 mL), sodium methanethiolate (203 mg; 2.89 mmol; 1.5 eq.) is added slowly and the resulting mixture is stirred at 85° C. for 2 h in a pressure vessel. Solvents are evaporated and the residue is partitioned between EtOAc and water. The organic phase is washed with brine, dried over Na₂SO₄ and concentrated in vacuo and co-evaporated a three times with hexane to give 3-methylsulfanyl pyrazin-2-ylamine (238 mg; 1.68 mmol; yield: 87%; beige powder; HPLC purity 99.5%).

### Intermediate 19, General Procedure 11

### 3-Methanesulfonyl-pyrazin-2-ylamine

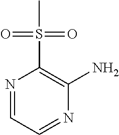

To a solution of 3-methylsulfanyl-pyrazin-2-ylamine (Intermediate 18, 320 mg; 2.27 mmol; 1 eq.) in MeOH (20 mL) at 0° C., an OXONE (1.39 g; 4.53 mmol; 2 eq.), solution in water (20 mL) is added dropwise. After 10 min., the reaction mixture is allowed to come back to room temperature and is left with stirring for 20 h. After evaporation of the volatiles, the aqueous layer is basified with aq. 1 N NaOH and extracted twice with EtOAc. The combined organic layers are washed with brine, dried over Na₂SO₄, filtered and concentrated in vacuo. The residue is purified by FCC (0% to 50% EtOAc gradient in hexane) to afford 3-methanesulfonyl-pyrazin-2-ylamine (196 mg; 1.13 mmol; yield: 50%; beige powder; UPLC purity 100%).

### Example 27

### N-(5-Methanesulfonylpyrimidin-4-yl)-8-(1-methyl-1H-indol-6-yl)quinoxalin-6-amine

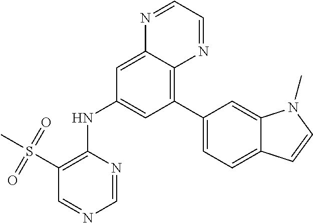 [SMILES: Cn1ccc2ccc(-c3cc(Nc4ncncc4S(C)(=O)=O)cc4nccnc34)cc21]

The title compound is prepared according to General Procedure 5 described in example 8 with 7-chloro-5-(1-methyl-1H-indol-6-yl)quinoxaline (Intermediate 2B, 40 mg; 0.14 mmol; 1 eq.), 3-methanesulfonyl-pyrazin-2-ylamine (Intermediate 19, 35 mg; 0.20 mmol; 1.5 eq.), tBuONa (39 mg; 0.41 mmol; 3 eq.), BippyPhos (28 mg; 0.05 mmol; 0.4 eq.) and (Pd(cinnamyl)Cl)₂ (7 mg; 0.01 mmol; 0.1 eq.) in toluene (2 mL). After 16 h at 110° C., further portions of 3-methanesulfonyl-pyrazin-2-ylamine (24 mg; 0.14 mmol; 1 eq.), (Pd(cinnamyl)Cl)₂ (7. mg; 0.01 mmol; 0.1 eq.) and BippyPhos (28 mg; 0.05 mmol; 0.4 eq.) are added and heating is continued for another 16 h. Purification by FCC (EtOAc gradient in hexane) followed by reversed-phase preparative HPLC (column: Gemini NX C18 5u 110A (100×30 mm), ACN gradient in water with 0.1% ammonia) to afford N-(5-methanesulfonylpyrimidin-4-yl)-8-(1-methyl-1H-indol-6-yl)quinoxalin-6-amine (16 mg; 0.03 mmol; yield: 24%; yellow powder; HPLC purity: 100%).

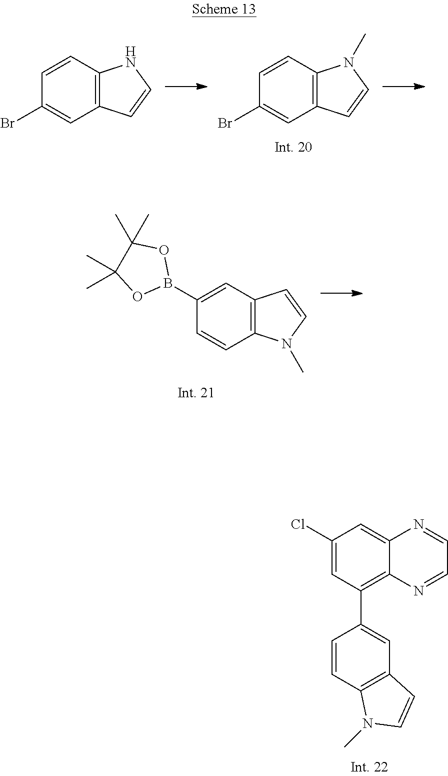 [SMILES: Brc1ccc2[nH]ccc2c1 ; Cn1ccc2cc(Br)ccc21 ; Cn1ccc2cc(B3OC(C)(C)C(C)(C)O3)ccc21 ; Cn1ccc2cc(-c3cc(Cl)cc4nccnc34)ccc21]

### Intermediate 20

### 5-Bromo-1-methyl-1H-indole

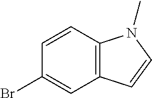

A solution of 5-bromo-1H-indole (20 g; 102 mmol; 1 eq.) in THF (80 mL) is cooled in an ice bath. NaH (60% in mineral oil, 8 g; 204 mmol; 2 eq.) is added portionwise and the reaction mixture is stirred for 30 min. MeI (8.3 mL; 132 mmol; 1.3 eq.) is added dropwise and the reaction mixture is stirred overnight at room temperature. The reaction mixture is poured onto ice and extracted twice with diethyl ether. The combined organic layers are washed with brine, dried over MgSO₄, filtered and evaporated in vacuo to provide 5-bromo-1-methyl-1H-indole (24 g; 102 mmol; yield: 99%; clear oil; UPLC purity 89%).

### Intermediate 21

### 1-Methyl-5-(4,4,5,5-tetramethyl-[1,3,2]dioxaborolan-2-yl)-1H-indole

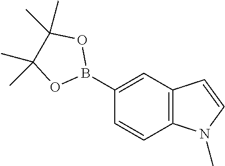 [SMILES: Cn1ccc2cc(B3OC(C)(C)C(C)(C)O3)ccc21]

5-Bromo-1-methyl-1H-indole (Intermediate 20, 24 g; 102 mmol; 1 eq.), 4,4,5,5,4′,4′,5′,5′-octamethyl-[2,2′]bis[[1,3,2]dioxaborolanyl] (34 g; 132 mmol; 1.3 eq.), dioxane (150 mL) and potassium acetate (20 g; 203 mmol; 2 eq.) are placed in a pressure vessel. The reaction mixture is sparged with argon before Pd(dppf)Cl₂ (744 mg; 1.02 mmol; 0.01 eq.) is added. The reaction vessel is sealed and the reaction mixture is stirred at 90° C. overnight. After coming back to room temperature, it is diluted with EtOAc/hexane 1/1 and filtered through a pad of celite. Silica (20 g) is added to the filtrate and the solvents are evaporated. The residue is purified by FCC (0% to 10% EtOAc gradient in hexane) to afford 1-methyl-5-(4,4,5,5-tetramethyl-[1,3,2]dioxaborolan-2-yl)-1H-indole (27 g; 88 mmol; yield 87%; off-white powder; UPLC purity: 84%).

### Intermediate 22

### 7-Chloro-5-(1-methyl-1H-indol-5-yl)quinoxaline

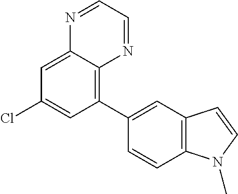 [SMILES: Cn1ccc2cc(-c3cc(Cl)cc4nccnc34)ccc21]

A pressure vessel is charged with 5-bromo-7-chloro-quinoxaline (Intermediate 2, 4 g; 16 mmol; 1 eq.), 1-methyl-5-(4,4,5,5-tetramethyl-[1,3,2]dioxaborolan-2-yl)-1H-indole (Intermediate 21, 5.13 g; 15.77 mmol; 1 eq.), cesium carbonate (10 g; 31.54 mmol; 2 eq.), 1,2-dimethoxyethane (30 mL) and water (15 mL). The reaction mixture is sparged with argon under sonication before Pd(dppf)Cl₂CH₂Cl₂ (0.66 g; 0.79 mmol; 0.05 eq.) is added. The tube is sealed and the reaction mixture is stirred for 1.5 h at 100° C. The reaction mixture is cooled to room temperature, diluted with EtOAc and washed with water and brine. The organic phase is dried over Na₂SO₄ and filtered through a pad of celite. The filtrate is concentrated in vacuo and the residue is purified by FCC (EtOAc gradient in hexane) and triturated with pentane to afford 7-chloro-5-(1-methyl-1H-indol-5-yl)-quinoxaline (2.94 g; 9.31 mmol; yield: 59%; UPLC purity: 93%).

### Example 28

### 3-{[8-(1-Methyl-1H-indol-5-yl)quinoxalin-6-yl]amino}pyridine-4-carbonitrile

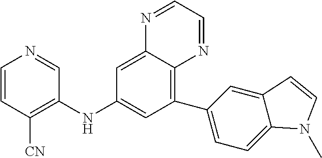 [SMILES: Cn1ccc2cc(-c3cc(Nc4cnccc4C#N)cc4nccnc34)ccc21]

The title compound is prepared according to General Procedure 1 described in Example 1, with 7-chloro-5-(1-methyl-1H-indol-5-yl)-quinoxaline (Intermediate 22, 220 mg; 0.75 mmol; 1 eq.), 3-aminoisonicotinonitrile (138 mg; 1.12 mmol; 1.50 eq.), cesium carbonate (739 mg; 2.25 mmol; 3 eq.), BINAP (48 mg; 0.07 mmol; 0.10 eq.) and Pd(OAc)₂ (18 mg; 0.07 mmol; 0.10 eq.) in dioxane (8 mL). Conditions: stirring at 150° C. for 1 h. Purification by FCC (50-80% EtOAc gradient in hexane) affords 3-{[8-(1-methyl-1H-indol-5-yl)quinoxalin-6-yl]amino}pyridine-4-carbonitrile (276 mg; 0.70 mmol; yield: 93%; yellow powder; HPLC purity: 95%).

### Example 29

### 3-{[8-(1-Methyl-1H-indol-5-yl)quinoxalin-6-yl]amino}pyridine-4-carboxamide

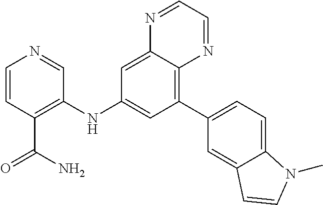 [SMILES: Cn1ccc2cc(-c3cc(Nc4cnccc4C(N)=O)cc4nccnc34)ccc21]

A round-bottom flask is charged with tBuOH (4 mL) and KOH (36 mg; 0.64 mmol; 3 eq.). After complete dissolution of KOH, 3-{[8-(1-methyl-1H-indol-5-yl)quinoxalin-6-yl]amino}pyridine-4-carbonitrile (Example 28, 80 mg; 0.21 mmol; 1 eq.) is added and the reaction mixture is stirred at 80° C. for 3 h, at which point TLC showed complete conversion of the starting material. The reaction mixture is diluted with EtOAc, neutralized with 1 M HCl, washed with water and saturated NH₄Cl. The organic layer is dried over Na₂SO₄ and filtered through a pad of celite and the filtrate is concentrated in vacuo. The crude product is purified by FCC (50-100% EtOAc gradient in hexane) followed by trituration with pentane to afford 3-{[8-(1-methyl-1H-indol-5-yl)quinoxalin-6-yl]amino}pyridine-4-carboxamide (36 mg; 0.09 mmol; yield: 42%; yellow powder; HPLC purity 98%).

### Example 30

### N-(4-Chloropyridin-3-yl)-8-(1-methyl-1H-indol-5-yl)quinoxalin-6-amine

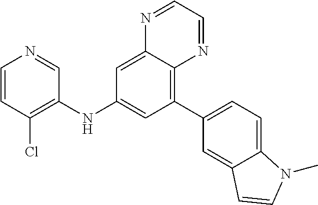 [SMILES: Cn1ccc2cc(-c3cc(Nc4cnccc4Cl)cc4nccnc34)ccc21]

The title compound is prepared according to General Procedure 5 described in Example 8 with 7-chloro-5-(1-methyl-1H-indol-5-yl)-quinoxaline (Intermediate 22, 120 mg; 0.38 mmol; 1 eq.), 4-chloro-pyridin-3-ylamine (51 mg; 0.40 mmol; 1.05 eq.), K₂CO₃ (105 mg; 0.76 mmol; 2 eq.), BippyPhos (38 mg; 0.08 mmol; 0.2 eq.), (Pd(cinnamyl)Cl)₂ (8 mg; 0.02 mmol; 0.04 eq.) in dioxane (3 mL). Conditions: 120° C., 24 h. Purification by FCC (EtOAc gradient in hexane, column neutralized with 1% ammonia in DCM and washed with DCM beforehand) affords N-(4-chloropyridin-3-yl)-8-(1-methyl-1H-indol-5-yl)quinoxalin-6-amine (20 mg; 0.05 mmol; yield: 13%; yellow-green powder; HPLC purity: 97.3%).

### Example 31, General Procedure 12

### 8-(1-Methyl-1H-indol-5-yl)-N-[4-(1-methyl-1H-pyrazol-4-yl)pyridin-3-yl]quinoxalin-6-amine

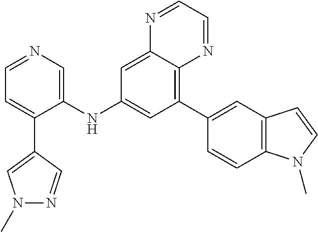 [SMILES: Cn1cc(-c2ccncc2Nc2cc(-c3ccc4c(ccn4C)c3)c3nccnc3c2)cn1]

A microwave tube is charged with N-(4-chloropyridin-3-yl)-8-(1-methyl-1H-indol-5-yl)quinoxalin-6-amine described in Example 30 (22 mg; 0.05 mmol; 1 eq.), 1-methyl-4-(4,4,5,5-tetramethyl-[1,3,2]dioxaborolan-2-yl)-1H-pyrazole (22 mg; 0.11 mmol; 2 eq.), potassium acetate (31 mg; 0.32 mmol; 6 eq.), acetonitrile (1 mL) and water (0.50 mL). The reaction mixture is sparged with argon before Pd(dppf)Cl₂ (10 mg; 0.01 mmol; 0.25 eq.) is added. The tube is sealed and the reaction mixture is heated under microwave irradiation at 140° C. for 1 h. It is then concentrated in vacuo and the residue is purified by FCC (0-10% MeOH gradient in DCM, column neutralized with 1% ammonia in DCM and washed with DCM beforehand) to afford 8-(1-methyl-1H-indol-5-yl)-N-[4-(1-methyl-1H-pyrazol-4-yl)pyridin-3-yl]quinoxalin-6-amine (12 mg; 0.03 mmol; yield 47%; yellow powder; HPLC purity: 90.1%).

### Example 32

### 8-(1-Methyl-1H-indol-5-yl)-N-[4-(4-methylpiperazin-1-yl)pyridin-3-yl]quinoxalin-6-amine

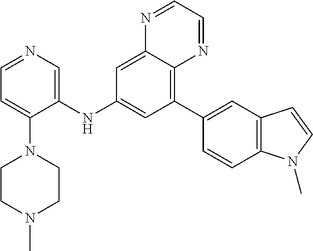 [SMILES: CN1CCN(c2ccncc2Nc2cc(-c3ccc4c(ccn4C)c3)c3nccnc3c2)CC1]

The title compound is prepared according to General Procedure 7 with N-(4-chloropyridin-3-yl)-8-(1-methyl-1H-indol-5-yl)quinoxalin-6-amine (Example 30, 20 mg; 0.05 mmol; 1 eq.), 1-methylpiperazine (7 mg; 0.07 mmol; 1.5 eq.), BrettPhos (2 mg; 3.7 μmol; 0.07 eq.), BrettPhos precatalyst (3 mg; 3.3 μmol; 0.07 eq.) and LiHMDS (1 M in THF, 0.20 mL; 0.20 mmol; 4 eq.). Conditions: 65° C. for 1.5 h. Purification by FCC (0-10% MeOH gradient in DCM) affords 8-(1-methyl-1H-indol-5-yl)-N-[4-(4-methylpiperazin-1-yl)pyridin-3-yl]quinoxalin-6-amine (14 mg; 0.03 mmol; yield 61%; yellow powder; HPLC purity: 97.5%).

### Example 33

### 8-(1-Methyl-1H-indol-5-yl)-N-[4-(pyrimidin-5-yl)pyridin-3-yl]quinoxalin-6-amine

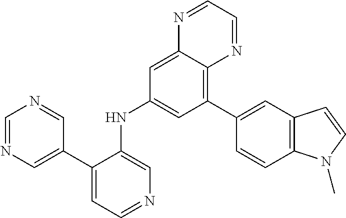 [SMILES: Cn1ccc2cc(-c3cc(Nc4cnccc4-c4cncnc4)cc4nccnc34)ccc21]

The title compound is prepared according to General Procedure 12 described in Example 31, with N-(4-chloropyridin-3-yl)-8-(1-methyl-1H-indol-5-yl)quinoxalin-6-amine (Example 30, 20 mg; 0.05 mmol; 1 eq.), 5-(4,4,5,5-tetramethyl-[1,3,2]dioxaborolan-2-yl)pyrimidine (21 mg; 0.10 mmol; 2 eq.), potassium acetate (30 mg; 0.30 mmol; 6 eq.), Pd(dppf)Cl₂ (10 mg; 0.01 mmol; 0.25 eq.) in acetonitrile (1 mL) and water (0.50 mL). Conditions: microwave irradiation, 140° C., 45 min. Purification by FCC (column: NH₂ 30 UM; MeOH gradient in DCM) affords 8-(1-methyl-1H-indol-5-yl)-N-[4-(pyrimidin-5-yl)pyridin-3-yl]quinoxalin-6-amine (10 mg; 0.02 mmol; yield 41%; yellow powder; HPLC purity: 87.8%).

### Example 34

### 5-(1-Methyl-1H-indol-5-yl)-7-{1H,2H,3H-pyrrolo[2,3-c]pyridin-1-yl}quinoxaline

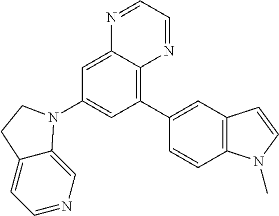 [SMILES: Cn1ccc2cc(-c3cc(N4CCc5ccncc54)cc4nccnc34)ccc21]

The title compound is prepared according to General Procedure 1 described in Example 1, with 7-chloro-5-(1-methyl-1H-indol-5-yl)quinoxaline (Intermediate 22, 60 mg; 0.20 mmol; 1 eq.), 2,3-dihydro-1H-pyrrolo[2,3-c]pyridine hydrochloride (0.06 mL; 0.31 mmol; 1.5 eq.), cesium carbonate (403 mg; 1.23 mmol; 6 eq.), BINAP (13 mg; 0.02 mmol; 0.1 eq.) and Pd(OAc)₂ (5 mg; 0.02 mmol; 0.1 eq.) in dioxane (8 mL). Conditions: 1 h, 150° C. Purification by FCC (0-100% EtOAc gradient in hexane then 0-10% MeOH gradient in EtOAc) affords 5-(1-methyl-1H-indol-5-yl)-7-{1H,2H,3H-pyrrolo[2,3-c]pyridin-1-yl}quinoxaline (20 mg; 0.05 mmol; yield: 25%; yellow solid; HPLC purity 98.1%).

### Example 35

### N-(2-Methanesulfonyl-5-nitrophenyl)-8-(1-methylindol-6-yl)quinoxalin-6-amine

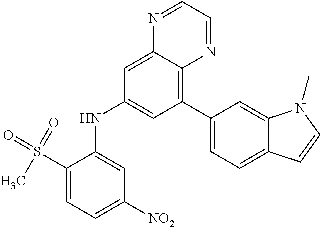 [SMILES: Cn1ccc2ccc(-c3cc(Nc4cc([N+](=O)[O-])ccc4S(C)(=O)=O)cc4nccnc34)cc21]

The title compound is prepared according to General Procedure 12 with 8-(1-methyl-1H-indol-6-yl)-quinoxalin-6-ylamine (Intermediate 4, 70 mg; 0.25 mmol; 1 eq.), 2-bromo-1-methanesulfonyl-4-nitrobenzene (78 mg; 0.27 mmol; 1.1 eq.), cesium carbonate (204 mg; ⁻0.62 mmol; 2.5 eq.), Pd(OAc)₂ (6 mg; 0.02 mmol; 0.1 eq.) and BINAP (16 mg; 0.02 mmol; 0.10 eq.) in dioxane (3 mL). After 1 h at 150° C., Pd(OAc)₂ (6 mg; 0.02 mmol; 0.1 eq.) and BINAP (16 mg; 0.02 mmol; 0.1 eq.) are added and stirring is continued at 125° C. for 3 h. Purification by FCC (EtOAc gradient in hexane) affords N-(2-methanesulfonyl-5-nitrophenyl)-8-(1-methylindol-6-yl)quinoxalin-6-amine (71 mg; 0.14 mmol; yield 57%; orange powder; HPLC purity: 95%).

### Example 36

### 6-Methanesulfonyl-N1-[8-(1-methyl-1H-indol-6-yl)quinoxalin-6-yl]benzene-1,3-diamine

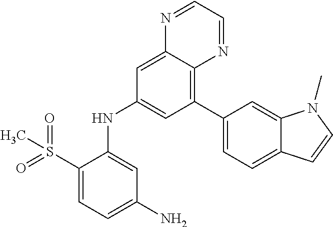 [SMILES: Cn1ccc2ccc(-c3cc(Nc4cc(N)ccc4S(C)(=O)=O)cc4nccnc34)cc21]

To a suspension of Raney Nickel (10 mg) in EtOH 96% (2 mL), hydrazine monohydrate (31 μL; 0.40 mmol; 5 eq.) is added dropwise followed by a suspension of N-(2-methanesulfonyl-5-nitrophenyl)-8-(1-methylindol-6-yl)quinoxalin-6-amine described in Example 35 (40 mg; 0.08 mmol; 1 eq.) in EtOH (96%, 1.50 mL). The reaction mixture is stirred at room temperature for 2 h at which point TLC showed complete conversion. The reaction mixture is diluted with DCM filtered through a pad of celite. Water is added to the filtrate and the aqueous layer is extracted twice with DCM. The combined organic layers are washed with brine, dried over Na₂SO₄ and concentrated in vacuo. Purification by FCC (0-100% EtOAc gradient in hexane, followed by 0-5%

MeOH gradient in EtOAc) affords 6-methanesulfonyl-N1-[8-(1-methyl-1H-indol-6-yl)quinoxalin-6-yl]benzene-1,3-diamine (26 mg; 0.06 mmol; 72%; yellow powder; HPLC purity: >99%).

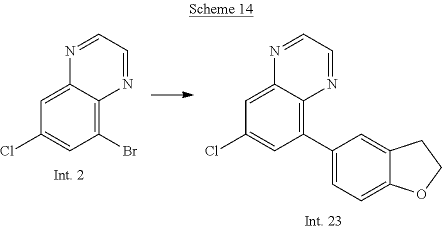 [SMILES: Clc1cc(Br)c2nccnc2c1 ; Clc1cc(-c2ccc3c(c2)CCO3)c2nccnc2c1]

### Intermediate 23

### 7-Chloro-5-(2,3-dihydro-1-benzofuran-5-yl)quinoxaline

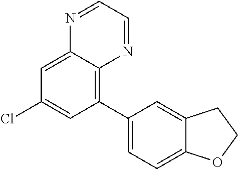 [SMILES: Clc1cc(-c2ccc3c(c2)CCO3)c2nccnc2c1]

A pressure vessel is charged with 5-bromo-7-chloro-quinoxaline (Intermediate 2 (288 mg, 1.18 mmol, 1 eq.), 5-(4,4,5,5-tetramethyl-[1,3,2]dioxaborolan-2-yl)-2,3-dihydrobenzofuran (306 mg; 1.18 mmol; 1 eq.), cesium carbonate (779 mg; 2.37 mmol; 2 eq.), 1,2-dimethoxyethane (10 mL) and water (5 mL). The reaction, mixture is sparged with argon under sonication before Pd(dppf)Cl₂CH₂Cl₂ (148 mg; 0.18 mmol; 0.15 eq.) is added. The tube is sealed and the reaction mixture is stirred at 100° C. for 1 h. After coming back to room temperature, the reaction mixture is partitioned between EtOAc and water. The aq. layer is extracted with EtOAc and the combined organic layers are washed with water and brine, dried over Na₂SO₄ filtered through a pad of celite and concentrated in vacuo. Purification of the residue by FCC (EtOAc gradient in hexane) affords 7-chloro-5-(2,3-dihydro-1-benzofuran-5-yl)quinoxaline (58 mg; 0.20 mmol; yield: 17%; off-white powder; UPLC purity: 97%).

### Example 37

### 8-(2,3-Dihydro-1-benzofuran-5-yl)-N-(4-methanesulfonylpyridin-3-yl)quinoxalin-6-amine

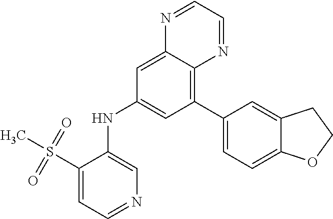 [SMILES: CS(=O)(=O)c1ccncc1Nc1cc(-c2ccc3c(c2)CCO3)c2nccnc2c1]

The title compound is prepared according to General Procedure 1 described in Example 1, with 7-chloro-5-(2,3-dihydro-benzofuran-5-yl)-quinoxaline (Intermediate 23, 40 mg; 0.14 mmol; 1 eq.), 4-methanesulfonylpyridin-3-ylamine hydrochloride (45 mg; 0.20 mmol; 1.5 eq.), cesium carbonate (223 mg; 0.68 mmol; 5 eq.), BINAP (9 mg; 0.01 mmol; 0.1 eq.) and Pd(OAc)₂ (3 mg; 0.01 mmol; 0.1 eq.) in dioxane (2 mL). Conditions: 1 h, 150° C. Purification by FCC (EtOAc gradient in hexane) affords 8-(2,3-dihydro-1-benzofuran-5-yl)-N-(4-methanesulfonylpyridin-3-yl)quinoxalin-6-amine (49 mg; 0.12 mmol; yield: 85%; pale yellow powder; HPLC purity: 98.5%).

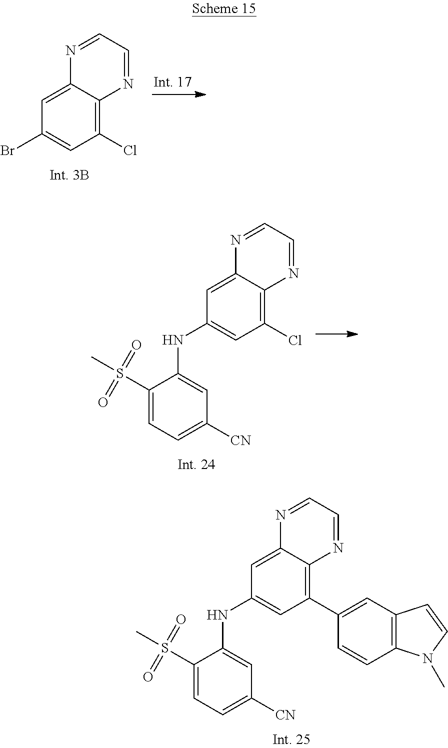 [SMILES: Clc1cc(Br)cc2nccnc12 ; CS(=O)(=O)c1ccc(C#N)cc1Nc1cc(Cl)c2nccnc2c1 ; Cn1ccc2cc(-c3cc(Nc4cc(C#N)ccc4S(C)(=O)=O)cc4nccnc34)ccc21]

### Intermediate 24

### 3-[(8-Chloroquinoxalin-6-yl)amino]-4-methanesulfonylbenzonitrile

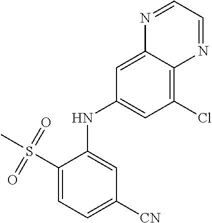 [SMILES: CS(=O)(=O)c1ccc(C#N)cc1Nc1cc(Cl)c2nccnc2c1]

A pressure vessel is charged with 7-bromo-5-chloroquinoxaline (Intermediate 3, 175 mg; 0.71 mmol; 1 eq.), 3-amino-4-methanesulfonylbenzonitrile (Intermediate 17, 153 mg, 0.78 mmol, 1.10 eq.), tBuONa (84 mg; 0.85 mmol; 1.2 eq.) and toluene (4 mL). The reaction mixture is sparged with argon under sonication before BINAP (18 mg; 0.03 mmol; 0.04 eq.) and Pd₂(dba)₃ (14 mg; 0.01 mmol; 0.02 eq.) are added. Tube is sealed and the reaction mixture is stirred at 120° C. for 50 minutes at which point TLC showed completion of the reaction. The reaction mixture is diluted with EtOAc, washed with water and brine, dried over Na₂SO₄, filtered through a pad of celite and concentrated in vacuo. The crude product is purified by FCC (0-4% MeOH gradient in DCM) to provide 3-[(8-chloroquinoxalin-6-yl)amino]-4-methanesulfonylbenzonitrile (168 mg; 0.47 mmol; yield: 66%; yellow powder; UPLC purity 92%).

### Intermediate 25

### 4-Methanesulfonyl-3-{[8-(1-methyl-1H-indol-5-yl)quinoxalin-6-yl]amino}benzonitrile

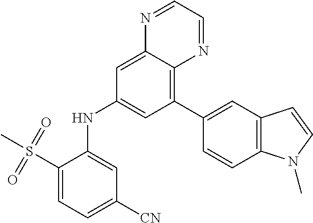 [SMILES: Cn1ccc2cc(-c3cc(Nc4cc(C#N)ccc4S(C)(=O)=O)cc4nccnc34)ccc21]

A microwave tube is charged with 3-[(8-chloroquinoxalin-6-yl)amino]-4-methanesulfonylbenzonitrile (Intermediate 24, 168 mg; 0.47 mmol; 1 eq.), 1-methyl-5-(4,4,5,5-tetramethyl-[1,3,2]dioxaborolan-2-yl)-1H-indole (Intermediate 21, 176 mg; 0.52 mmol; 1.1 eq.), sodium carbonate aq. solution (2 M, 0.47 mL; 0.94 mmol; 2 eq.) and dioxane (5 mL).: The reaction mixture is sparged with argon and Pd(PPh₃)₄ (27 mg; 0.02 mmol; 0.05 eq.) is added. The reaction tube is sealed and heated at 120° C. under microwave irradiation for 45 min. Additional cycles of heating under microwave irradiation at 130° C. for 45 and 30 min insured complete conversion. The reaction mixture is diluted with DCM and filtered through a pad of celite. The filtrate is washed with water and brine, dried over Na₂SO₄, filtered and concentrated in vacuo. The residue is purified by FCC (0-50% EtOAc gradient in hexane) to afford 4-methanesulfonyl-3-{[8-(1-methyl-1H-indol-5-yl)quinoxalin-6-yl]amino}benzonitrile (189 mg; 0.39 mmol; yield: 82%; yellow powder; UPLC purity: 93%).

### Example 38

### N-[5-(Aminomethyl)-2-methanesulfonylphenyl]-8-(1-methyl-1H-indol-5-yl)quinoxalin-6-amine

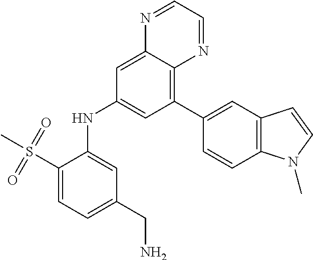 [SMILES: Cn1ccc2cc(-c3cc(Nc4cc(CN)ccc4S(C)(=O)=O)cc4nccnc34)ccc21]

To a cooled solution of 4-methanesulfonyl-3-{[8-(1-methyl-1H-indol-5-yl)quinoxalin-6-yl]amino}benzonitrile (Intermediate 25, 38 mg; 0.08 mmol; 1 eq.) in THF (freshly distilled over sodium/benzophenone, 1 mL) lithium aluminium hydride (2 M in THF, 0.12 mL; 0.23 mmol; 3 eq.) is added. The reaction mixture is stirred at room temperature for 3 h and carefully quenched by addition of water (10 μL) at 0° C. followed by 10 μL of 15% NaOH. The reaction mixture is further diluted with water and allowed to warm to room temperature with stirring for 30 min. It is then filtered through a pad of Celite and the filter cake is washed thoroughly with DCM. The aq. layer of the resulting filtrate is extracted with DCM and the combined organic layers are washed with water and brine, dried over Na₂SO₄ and concentrated in vacuo. Purification by FCC (Puriflash NH₂ 30 UM 20G column, EtOAc gradient in hexane then 0-5% MeOH gradient in EtOAc) affords N-[5-(aminomethyl)-2-methanesulfonylphenyl]-8-(1-methyl-1H-indol-5-yl)quinoxalin-6-amine (8 mg; 0.02 mmol; yield: 20%; yellow solid; HPLC purity: 88%).

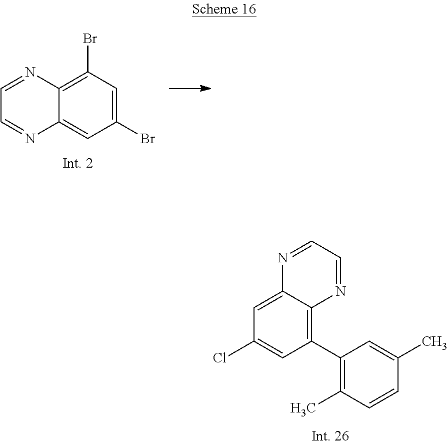 [SMILES: Brc1cc(Br)c2nccnc2c1 ; Cc1ccc(C)c(-c2cc(Cl)cc3nccnc23)c1]

### Intermediate 26

### 7-Chloro-5-(2,5-dimethyl-phenyl)quinoxaline

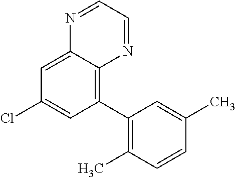 [SMILES: Cc1ccc(C)c(-c2cc(Cl)cc3nccnc23)c1]

A pressure vessel is charged with 5-bromo-7-chloro-quinoxaline (Intermediate 2, 100 mg; 0.39 mmol; 1 eq.), (2,5-dimethylphenyl)boronic acid (59 mg; 0.39 mmol; 1 eq.), cesium carbonate (257 mg; 0.79 mmol; 2 eq.), 1,2-dimethoxyethane (5 mL) and water (1.5 mL). The reaction mixture is sparged with argon under sonication before Pd(dppf)Cl₂CH₂Cl₂ (32 mg; 0.04 mmol; 0.10 eq.) is added. The tube is sealed and the reaction mixture is stirred for 1 h at 100° C. After cooling to room temperature, the reaction mixture is diluted with EtOAc and washed with water and brine. The organic layer is dried over Na₂SO₄ and filtered through a pad of celite, and the filter cake is washed with EtOAc. The filtrate is concentrated in vacuo and the residue is purified by FCC (0-15% EtOAc gradient in hexane) to afford 7-chloro-5-(2,5-dimethyl-phenyl)-quinoxaline (74 mg, 0.27 mmol; yield: 69.1%; UPLC purity: 99%).

### Example 39

### 8-(2,5-Dimethylphenyl)-N-(4-methanesulfonylpyridin-3-yl)quinoxalin-6-amine

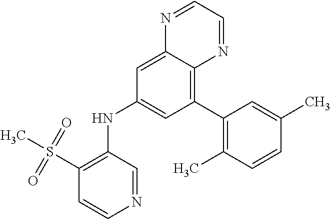

The title compound is prepared according to General Procedure 2 with 7-chloro-5-(2,5-dimethyl-phenyl)quinoxaline (Intermediate 26, 74 mg; 0.28 mmol; 1 eq.), 4-methanesulfonylpyridin-3-ylamine hydrochloride (115 mg; 0.55 mmol; 2 eq.), tBuONa (132 mg; 1.38 mmol; 5 eq.), BINAP (17 mg; 0.03 mmol; 0.10 eq.) and Pd₂(dba)₃ (13 mg; 0.01 mmol; 0.05 eq.) in toluene (2 mL). Conditions: 110° C., 16 hours. Purification by FCC (0-100% EtOAc gradient in hexane then 0-10% MeOH gradient in EtOAc) affords 8-(2,5-dimethylphenyl)-N-(4-methanesulfonylpyridin-3-yl)quinoxalin-6-amine (46 mg; 0.11 mmol; yield: 41%; yellow powder; HPLC purity: 99%).

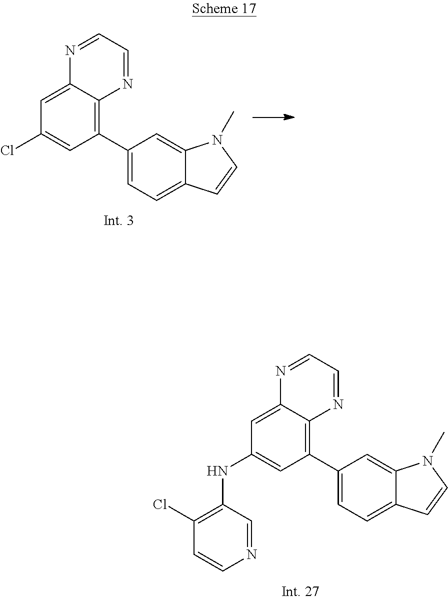 [SMILES: Cn1ccc2ccc(-c3cc(Cl)cc4nccnc34)cc21 ; Cn1ccc2ccc(-c3cc(Nc4cnccc4Cl)cc4nccnc34)cc21]

### Intermediate 27

### N-(4-Chloropyridin-3-yl)-8-(1-methyl-1H-indol-6-yl)quinoxalin-6-amine

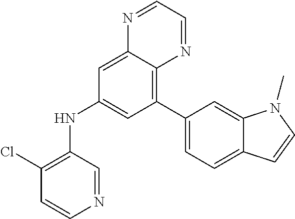 [SMILES: Cn1ccc2ccc(-c3cc(Nc4cnccc4Cl)cc4nccnc34)cc21]

The title compound is prepared according to General Procedure 5 described in Example 8 with 7-chloro-5-(1-methyl-1H-indol-6-yl)quinoxaline (Intermediate 2B, 160 mg; 0.54 mmol; 1 eq.), 4-chloro-pyridin-3-ylamine (83 mg; 0.65 mmol; 1.2 eq.), K₂CO₃ (186 mg; 1.35 mmol; 2.5 eq.), BippyPhos (55 mg; 0.11 mmol; 0.20 eq.) and (Pd(cinnamyl)Cl)₂ (11 mg; 0.02 mmol; 0.04 eq.) in dioxane (2 mL). Conditions: 120° C., 24 h. Purification by FCC (column neutralized with 1% Et₃N in DCM beforehand, 0-100% EtOAc gradient in hexane then 0-30% MeOH gradient in EtOAc) affords N-(4-chloropyridin-3-yl)-8-(1-methyl-1H-indol-6-yl)quinoxalin-6-amine (23 mg; 0.04 mmol; yield: 8%; yellow powder; UPLC purity: 72%).

### Example 40

### 8-(1-Methyl-1H-indol-6-yl)-N-[4-(4-methylpiperazin-1-yl)pyridin-3-yl]quinoxalin-6-amine

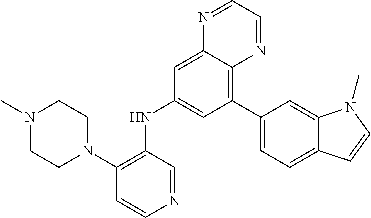 [SMILES: CN1CCN(c2ccncc2Nc2cc(-c3ccc4ccn(C)c4c3)c3nccnc3c2)CC1]

The title compound is prepared according to General Procedure 7 with N-(4-chloropyridin-3-yl)-8-(1-methyl-1H-indol-6-yl)quinoxalin-6-amine (Intermediate 27, 23 mg; 0.04 mmol; 1 eq.), 1-methylpiperazine (7 mg; 0.07 mmol; 1.50 eq.), BrettPhos (2 mg; 3.7 mmol; 0.07 eq.), BrettPhos Pd G1 methyl-t-butyl ether adduct (3 mg; 3.3 μmol; 0.07 eq.) and LiHMDS (1 M THF solution, 0.18 mL; 0.18 mmol; 4 eq.), Conditions: 65° C., 2 h. Purification by FCC (0-10% MeOH gradient in DCM) affords 8-(1-methyl-1H-indol-6-yl)-N-[4-(4-methylpiperazin-1-yl)pyridin-3-yl]quinoxalin-6-amine (12 mg; 0.03 mmol; yield: 58%; brown-yellow powder; HPLC purity: 97.5%).

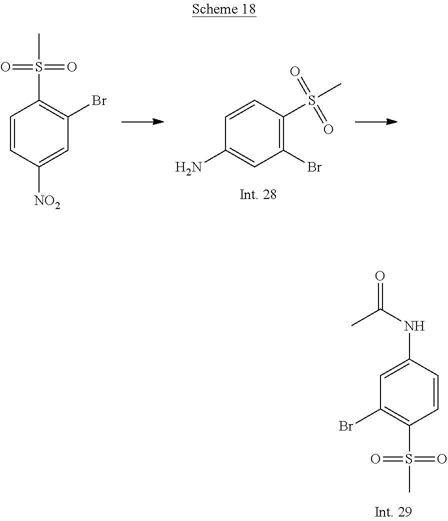

### Intermediate 28

### 3-Bromo-4-methanesulfonyl-phenylamine

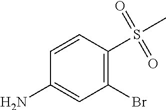

A solution of tin(II) chloride dihydrate (197 mg; 0.87 mmol; 5 eq.) in EtOAc (2 mL) is heated under reflux for 5 min. and 2-bromo-1-methanesulfonyl-4-nitro-benzene (50 mg; 0.17 mmol; 1 eq.) is added in one portion. Heating is continued for 3 h. The reaction mixture is concentrated in vacuo and the residue is suspended in DCM and treated with a solution of NaOH (350 mg; 8.75 mmol; 50 eq.) in water (1 mL). The resulting mixture is stirred at room temperature until complete dissolution of the white tin precipitate. The organic layer is washed with water and brine, dried over Na₂SO₄ and concentrated to give 3-bromo-4-methanesulfonylphenylamine (38 mg; 0.15 mmol; yield: 85%; off-white solid; UPLC purity: 97.5%).

### Intermediate 29

### N-(3-Bromo-4-methanesulfonyl-phenyl)acetamide

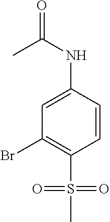

To a cooled solution of 3-bromo-4-methanesulfonylphenylamine (Intermediate 28, 37 mg; 0.14 mmol; 1 eq.) in DCM (2 mL) are added triethylamine (37 μL; 0.29 mmol; 2 eq.) and acetyl chloride (15 μL; 0.20 mmol; 1.4 eq.). The reaction mixture is sonicated at room temperature for 30 min. and a new portion of acetyl chloride (15 μL; 0.20 mmol; 1.4 eq.) is added. The reaction mixture is stirred at room temperature for 2 h before another portion of acetyl chloride (15 μL; 0.20 mmol; 1.4 eq.) is added. After an additional hour, complete conversion of the starting material is confirmed by TLC. Water is added to the reaction mixture and the product is extracted twice with DCM. The combined organic phases are washed with brine, dried over Na₂SO₄, filtered and concentrated in vacuo to give N-(3-Bromo-4-methanesulfonyl-phenyl)acetamide (39 mg; 0.13 mmol; yield: 90%; light brown solid; UPLC purity: 97.5%).

### Example 41

### N-(4-Methanesulfonyl-3-{[8-(1-methyl-1H-indol-6-yl)quinoxalin-6-yl]amino}phenyl)acetamide

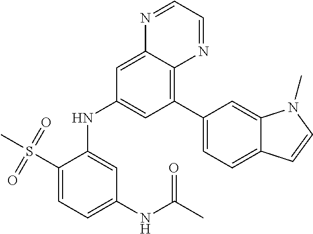 [SMILES: CC(=O)Nc1ccc(S(C)(=O)=O)c(Nc2cc(-c3ccc4ccn(C)c4c3)c3nccnc3c2)c1]

The title compound is prepared according to General Procedure 1 described in Example 1, with 8-(1-methyl-1H-indol-6-yl)-quinoxalin-6-ylamine (Intermediate 4, 40 mg; 0.14 mmol; 1 eq.), N-(3-Bromo-4-methanesulfonyl-phenyl)acetamide (Intermediate 29, 38 mg; 0.13 mmol; 0.9 eq.), cesium carbonate (93 mg; 0.28 mmol; 2 eq.), BINAP (18 mg; 0.03 mmol; 0.20 eq.) and Pd(OAc)₂ (7 mg; 0.03 mmol; 0.20 eq.) in dioxane (2 mL). Conditions: 120° C., 5 h. Purification by FCC (EtOAc gradient in hexane) affords N-(4-methanesulfonyl-3-{[8-(1-methyl-1H-indol-6-yl)quinoxalin-6-yl]amino}phenyl)acetamide (40 mg; 0.08 mmol; yield: 55%; yellow powder; HPLC purity: 94%).

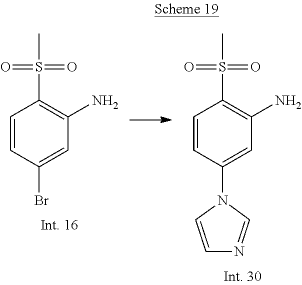 [SMILES: CS(=O)(=O)c1ccc(Br)cc1N ; CS(=O)(=O)c1ccc(-n2ccnc2)cc1N]

### Intermediate 30

### 5-(1H-Imidazol-1-yl)-2-methanesulfonylaniline

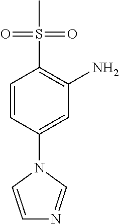 [SMILES: CS(=O)(=O)c1ccc(-n2ccnc2)cc1N]

A glass pressure reactor is charged with 5-bromo-2-methanesulfonyl-phenylamine (Intermediate 16, 90 mg; 0.36 mmol; 1 eq.), 1H-imidazole (124 mg; 1.80 mmol; 5 eq.) and KOH (149 mg; 2.52 mmol; 7 eq.) in DMSO (1.5 mL). The tube is sealed and the reaction mixture is stirred at 130° C. for 16 h. The reaction mixture is diluted with EtOAc, the solution is washed with water and brine, dried over Na₂SO₄ and concentrated in vacuo. The residue is purified by FCC (0-10% MeOH gradient in EtOAc) to yield 5-(1H-imidazol-1-yl)-2-methanesulfonylaniline (60 mg; 0.19 mmol; yield: 53%; light beige oil; UPLC purity: 75%).

### Example 42

### N-[5-(1H-Imidazol-1-yl)-2-methanesulfonylphenyl]-8-(1-methyl-1H-indol-6-yl)quinoxalin-6-amine

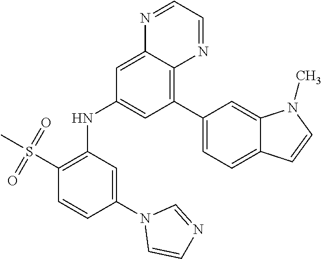 [SMILES: Cn1ccc2ccc(-c3cc(Nc4cc(-n5ccnc5)ccc4S(C)(=O)=O)cc4nccnc34)cc21]

The title compound is synthesized according to general procedure 1 with 7-chloro-5-(1-methyl-1H-indol-6-yl)quinoxaline (55 mg; 0.17 mmol; 1 eq.), 5-(1H-imidazol-1-yl)-2-methanesulfonylaniline (Intermediate 30, 58 mg; 0.18 mmol; 1.1 eq.), cesium carbonate (77 mg; 0.23 mmol; 1.4 eq.), BINAP (11 mg; 0.02 mmol; 0.10 eq.) and Pd(OAc)₂ (4 mg; 0.02 mmol; 0.1 eq.) in dioxane (2 mL). Conditions: 150° C. for 1 hour (pre-heated bath). Purification by FCC (0-20% MeOH gradient in EtOAc) affords N-[5-(1H-imidazol-1-yl)-2-methanesulfonylphenyl]-8-(1-methyl-1H-indol-6-yl)quinoxalin-6-amine (55 mg; 0.10 mmol; yield: 61%; light yellow powder; HPLC purity: 91.5%).

### Example 43

### N-[2-Methanesulfonyl-5-(2H-1,2,3,4-tetrazol-5-yl)phenyl]-8-(1-methyl-1H-indol-6-yl)quinoxalin-6-amine

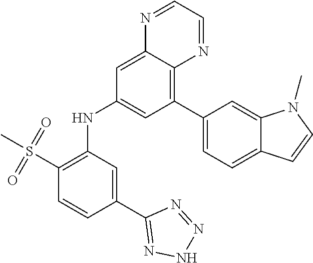 [SMILES: Cn1ccc2ccc(-c3cc(Nc4cc(-c5nn[nH]n5)ccc4S(C)(=O)=O)cc4nccnc34)cc21]

A pressure vessel is loaded with 4-methanesulfonyl-3-{[8-(1-methyl-1H-indol-6-yl)quinoxalin-6-yl]amino}benzonitrile (Example 25, 80 mg; 0.16 mmol; 1 eq.), sodium azide (32 mg; 0.48 mmol; 3 eq.), triethylamine hydrochloride (67 mg; 0.48 mmol; 3 eq.) and anhydrous toluene (3 mL) under Ar. The reactor is sealed and the reaction mixture is stirred at 110° C. for 16 h. The reaction mixture is concentrated and the residue is purified by FCC (0-20% MeOH gradient in DCM). After evaporation of the relevant fractions, the residue is dissolved in EtOAc and the solution is washed with water to remove traces of triethylamine hydrochloride. The organic phase is dried over Na₂SO₄, filtered and concentrated in vacuo to give N-[2-methanesulfonyl-5-(2H-1,2,3,4-tetrazol-5-yl)phenyl]-8-(1-methyl-1H-indol-6-yl)quinoxalin-6-amine (73 mg; 0.13 mmol; yield: 83%; yellow powder; HPLC purity: 91%).

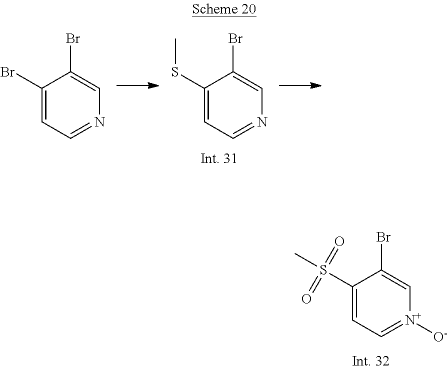

### Intermediate 31

### 3-Bromo-4-(methylsulfanyl)pyridine

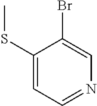

To a solution of 3,4-dibromopyridine (500 mg; 2.11 mmol; 1 eq.) in dry DMF (2 mL) sodium methanethionalate (163 mg; 2.32 mmol; 1.1 eq.) is added portionwise under Ar. After stirring at room temperature for 1 h, the reaction mixture is diluted with EtOAc and the organic layer is washed with water and brine, dried over Na₂SO₄ and concentrated in vacuo. The residue is purified by FCC (0-50% EtOAc gradient in hexane) to give 3-bromo-4-(methylsulfanyl)pyridine (370 mg; 1.81 mmol; yield: 86%; light yellow oil; UPLC purity: 100%).

### Intermediate 32

### 3-Bromo-4-methanesulfonylpyridin-1-ium-1-olate

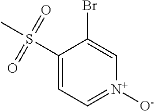

A mixture of 3-bromo-4-(methylsulfanyl)pyridine (Intermediate 31, 370 mg; 1.63 mmol; 1 eq.) and 3-chloroperoxybenzoic acid (1.1 g; 5.06 mmol; 3.1 eq.) in DCM (10 mL) is stirred at room temperature overnight. The mixture is diluted with DCM and washed with saturated aq. NaHCO₃, 1 M NaOH and brine. The organic phase is dried over MgSO₄, filtered and concentrated in vacuo. The residue is purified by FCC (EtOAc gradient in hexane then 0-10% MeOH gradient in EtOAc) to give 3-bromo-4-methanesulfonylpyridine (160 mg; 0.66 mmol; yield 41%; white powder; UPLC purity: 97.8%) and the title compound, 3-bromo-4-methanesulfonylpyridin-1-ium-1-olate (144 mg; 0.57 mmol; yield 35%; white powder; UPLC purity: 99.5%).

### Example 44

### 4-Methanesulfonyl-3-{[8-(1-methyl-1H-indol-6-yl)quinoxalin-6-yl]amino}pyridin-1-ium-1-olate

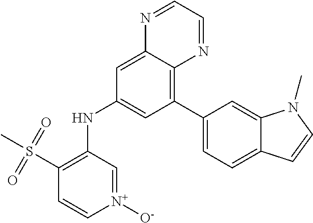 [SMILES: Cn1ccc2ccc(-c3cc(Nc4c[n+]([O-])ccc4S(C)(=O)=O)cc4nccnc34)cc21]

The title compound is prepared according to General Procedure 1 described in Example 1, with 3-bromo-4-methanesulfonylpyridin-1-ium-1-olate (Intermediate 32, 70 mg; 0.28 mmol; 1 eq.), 8-(1-methyl-1H-indol-6-yl)quinoxalin-6-ylamine (Intermediate 4, 94 mg; 0.33 mmol; 1.2 eq.), cesium carbonate (218 mg; 0.66 mmol; 2.4 eq.), BINAP (35 mg; 0.06 mmol; 0.2 eq.) and Pd(OAc)₂ (13 mg; 0.06 mmol; 0.1 eq.) in dioxane (2 mL). Conditions: 120° C., 5 h. Purification by FCC (0-20% MeOH gradient in EtOAc) gives 4-methanesulfonyl-3-{[8-(1-methyl-1H-indol-6-yl)quinoxalin-6-yl]amino}pyridin-1-ium-1-olate (45 mg; 0.10 mmol; yield: 35%; light yellow solid; HPLC purity: 97.2%).

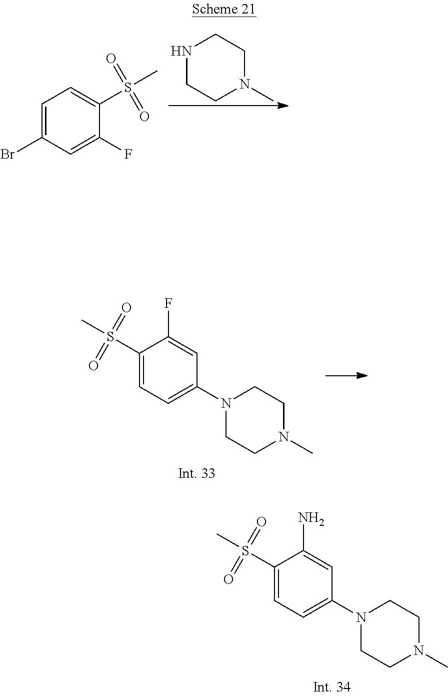

### Intermediate 33

### 1-(3-Fluoro-4-methanesulfonylphenyl)-4-methylpiperazine

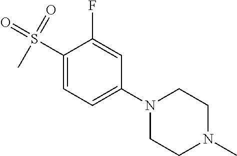 [SMILES: CN1CCN(c2ccc(S(C)(=O)=O)c(F)c2)CC1]

A pressure vessel is charged with 4-bromo-2-fluoro-1-methanesulfonyl-benzene (500 mg; 1.98 mmol; 1 eq.), 1-methylpiperazine (0.26 mL; 2.37 mmol; 1.2 eq.), BINAP (246 mg; 0.40 mmol; 0.20 eq.), cesium carbonate (2.57 g; 7.90 mmol; 4 eq.) and dioxane (25 mL). The resulting suspension is degassed by argon bubbling and sonication before Pd(OAc)₂ (44 mg; 0.20 mmol; 0.10 eq.) is added. The flask is sealed and the mixture is stirred at 100° C. for 1 hour, at which point TLC shows complete conversion of the starting material. The mixture is filtered through a pad of celite and the filter cake is washed with DCM. The filtrate is washed with brine, dried over Na₂SO₄, filtered and evaporated. The crude product is purified by 2 successive FFCs (first: 0-30% EtOAc gradient in hexanes, second: 1-5% MeOH gradient in EtOAc) to give 1-(3-fluoro-4-methanesulfonylphenyl)-4-methylpiperazine (253 mg; 0.83 mmol; yield: 42%; white powder; UPLC purity: 89%).

### Intermediate 34

### 2-Methanesulfonyl-5-(4-methylpiperazin-1-yl)aniline

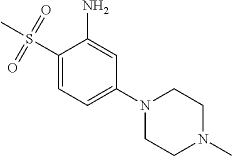

1-(3-Fluoro-4-methanesulfonylphenyl)-4-methylpiperazine (Intermediate 33, 100 mg; 0.33 mmol; 1 eq.) and DMSO (2 mL) are placed in a glass pressure reactor, aqueous ammonia (25%, 2 mL; 13.1 mmol; 40 eq.) is added, the reactor is sealed and the reaction mixture is stirred at 140° C. for 3 days. The reaction mixture is partitioned between EtOAc and water and the organic phase is washed with brine (twice), dried with Na₂SO₄ and concentrated in vacuo. The crude product is purified by FCC (0% to 10% MeOH gradient in DCM) to afford 2-methanesulfonyl-5-(4-methylpiperazin-1-yl)aniline (31 mg; 0.11 mmol; yield 34%; white powder; UPLC purity: 96%).

### Example 45

### N-[2-Methanesulfonyl-5-(4-methylpiperazin-1-yl)phenyl]-8-(1-methyl-1H-indol-6-yl)quinoxalin-6-amine

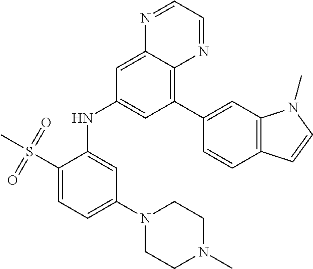 [SMILES: CN1CCN(c2ccc(S(C)(=O)=O)c(Nc3cc(-c4ccc5ccn(C)c5c4)c4nccnc4c3)c2)CC1]

A pressure vessel is loaded with 7-Chloro-5-(1-methyl-1H-indol-6-yl)quinoxaline (Intermediate 2B, 27 mg; 0.09 mmol; 1 eq.), 2-methanesulfonyl-5-(4-methylpiperazin-1-yl)aniline (Intermediate 34, 30 mg; 0.11 mmol; 1.20 eq.), BINAP (11 mg; 0.02 mmol; 0.20 eq.), Cs₂CO₃ (120 mg; 0.37 mmol; 4 eq.) and dioxane (1 mL). The resulting suspension is degassed by argon bubbling and sonication before Pd(OAc)₂ (2 mg; 0.01 mmol; 0.10 eq.) is added. The tube is sealed and the mixture is stirred at 90° C. for 1 hour, at which point TLC shows complete conversion of 7-Chloro-5-(1-methyl-1H-indol-6-yl)quinoxaline. The reaction mixture is filtered through a pad of celite and the filtrate cake is washed with DCM. The filtrate is washed with brine, dried over Na₂SO₄, filtered and concentrated. The crude is purified by FCC (0-10% MeOH in DCM) to yield N-[2-methanesulfonyl-5-(4-methylpiperazin-1-yl)phenyl]-8-(1-methyl-1H-indol-6-yl)quinoxalin-6-amine (28 mg; 0.05 mmol; yield: 56%; light yellow powder; HPLC purity: 97%).

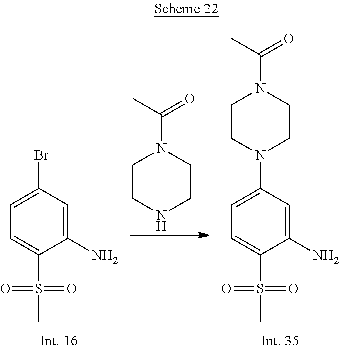

### Intermediate 35

### 1-[4-(3-Amino-4-methanesulfonylphenyl)piperazin-1-yl]ethan-1-one

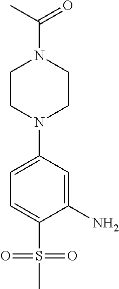 [SMILES: CC(=O)N1CCN(c2ccc(S(C)(=O)=O)c(N)c2)CC1]

A 5-mL microwave vial is charged with 5-bromo-2-methanesulfonyl-phenylamine (150 mg; 0.60 mmol; 1 eq.) and 1-piperazin-1-yl-ethanone (384 mg; 3 mmol; 5 eq.). The atmosphere is replaced by argon, the tube is sealed and the mixture is left with stirring at 130° C. over the weekend. After coming back to room temperature, saturated aq. bicarbonate (5 mL) is added and the mixture is vigorously stirred for 20 min. The resulting white solid is filtered off, washed with water and dried by azeotropic co-evaporation with toluene. The residue is taken up in DCM and purified by FCC (0-5% MeOH gradient in DCM). The fractions containing the pure compound are pooled, evaporated and the resulting solid is triturated in hexane, filtered and dried to afford 1--[4-(3-amino-4-methanesulfonylphenyl)piperazin-1-yl]ethan-1-one (85 mg; 0.29 mmol; yield: 48%; white powder; UPLC purity: 100%).

### Example 46

### 1-[4-(4-Methanesulfonyl-3-{[8-(1-methyl-1H-indol-6-yl)quinoxalin-6-yl]amino}phenyl)piperazin-1-yl]ethan-1-one

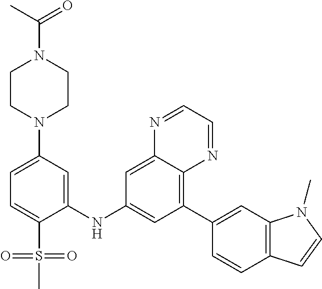 [SMILES: CC(=O)N1CCN(c2ccc(S(C)(=O)=O)c(Nc3cc(-c4ccc5ccn(C)c5c4)c4nccnc4c3)c2)CC1]

The title compound is prepared according to General Procedure 1 described in Example 1, with 7-chloro-5-(1-methyl-1H-indol-6-yl)quinoxaline (Intermediate 2B, 42 mg; 0.14 mmol; 1 eq.), 1-[4-(3-amino-4-methanesulfonylphenyl)piperazin-1-yl]ethan-1-one (Intermediate 35, 51 mg; 0.17 mmol; 1.20 eq.), BINAP (18 mg; 0.03 mmol; 0.20 eq.), Cs₂CO₃ (190 mg; 0.57 mmol; 4 eq.), dioxane (2 mL) and Pd(OAc)₂ (3 mg; 0.01 mmol; 0.10 eq.). Conditions: 90° C. for 1 hour. Purification by FCC (0%-20% MeOH gradient in EtOAc) to afford 1-[4-(4-methanesulfonyl-3-{[8-(1-methyl-1H-indol-6-yl)quinoxalin-6-yl]amino}phenyl)piperazin-1-yl]ethan-1-one (76 mg; 0.13 mmol; yield: 93%; white yellow powder; HPLC purity: 97%).

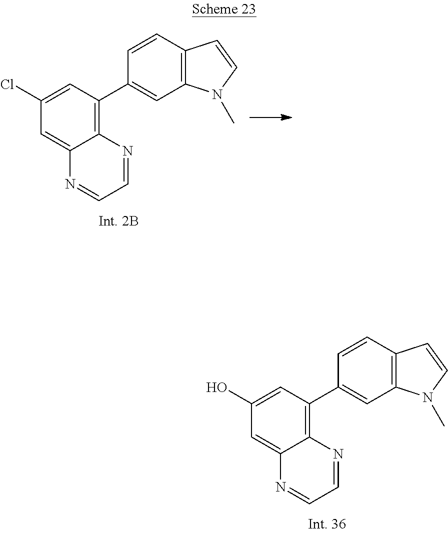 [SMILES: Cn1ccc2ccc(-c3cc(Cl)cc4nccnc34)cc21 ; Cn1ccc2ccc(-c3cc(O)cc4nccnc34)cc21]

### Intermediate 36

### 8-(1-Methyl-1H-indol-6-yl)quinoxalin-6-ol

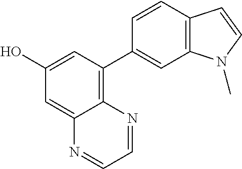 [SMILES: Cn1ccc2ccc(-c3cc(O)cc4nccnc34)cc21]

A pressure vessel is charged with 7-Chloro-5-(1-methyl-1H-indol-6-yl)quinoxaline (Intermediate 2B, 50 mg; 0.16 mmol; 1 eq.), K₂CO₃ (66 mg; 0.48 mmol; 3 eq.), tBuXPhos (11 mg; 0.03 mmol; 0.16 eq.), DMF (1 mL) and water (1 mL) The reaction mixture is sparged with argon, then Herrmann's palladacycle (6 mg; 0.01 mmol; 0.04 eq.) is added. The reaction tube is sealed and the reaction mixture is heated under microwave irradiation at 115° C. for 0.5 hour. The residue obtained after evaporation of volatiles is purified by FCC (5-50% EtOAc gradient in hexane) to obtain 8-(1-methyl-1H-indol-6-yl)quinoxalin-6-ol (38 mg; 0.13 mmol; yield 83%; orange brown solid; UPLC purity: 96%).

### Example 47

### 3-{[8-(1-Methyl-1H-indol-6-yl)quinoxalin-6-yl]oxy}pyridine-4-carbonitrile

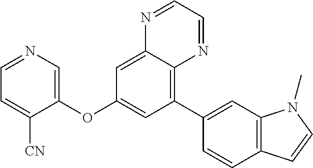 [SMILES: Cn1ccc2ccc(-c3cc(Oc4cnccc4C#N)cc4nccnc34)cc21]

A pressure vessel is loaded with 8-(1-methyl-1H-indol-6-yl)quinoxalin-6-ol (Intermediate 36, 18 mg; 0.06 mmol; 1 eq.), 3-chloroisonicotinonitrile (17 mg; 0.12 mmol; 2 eq.), tBuOK (9 mg; 0.09 mmol; 1.50 eq.) and DMSO (2 mL). The reaction mixture is stirred at 150° C. for 12 h. The solvent is evaporated, the residue is taken up in a small amount of DCM and purified by FCC (EtOAc gradient in hexane) to afford 3-{[8-(1-methyl-1H-indol-6-yl)quinoxalin-6-yl]oxy}pyridine-4-carbonitrile (14 mg; 0.03 mmol; yield: 56%; yellow powder; HPLC purity: 89%).

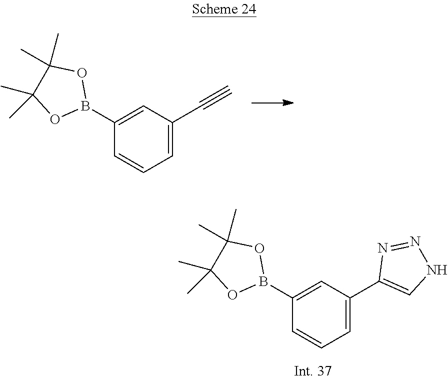 [SMILES: C#Cc1cccc(B2OC(C)(C)C(C)(C)O2)c1 ; CC1(C)OB(c2cccc(-c3c[nH]nn3)c2)OC1(C)C]

### Intermediate 37

### 4-[3-(Tetramethyl-1,3,2-dioxaborolan-2-yl)phenyl]-1H-1,2,3-triazole

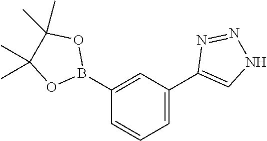 [SMILES: CC1(C)OB(c2cccc(-c3c[nH]nn3)c2)OC1(C)C]

To a stirred solution of 2-(3-ethynylphenyl)-4,4,5,5-tetramethyl-[1,3,2]dioxaborolane (250 mg; 1.10 mmol; 1 eq.) and CuI (11 mg; 0.05 mmol; 0.05 eq.) in DMF (1.80 mL) and MeOH (0.20 mL) under an argon atmosphere, azido(trimethyl)silane (1.9 g; 1.64 mmol; 1.50 eq.) is added. The resulting solution is stirred at 100° C. for 18 h. The reaction mixture is diluted with EtOAc and washed with water. The organic layer is washed with brine, dried over Na₂SO₄, filtered and concentrated to give 4-[3-(tetramethyl-1,3,2-dioxaborolan-2-yl)phenyl]-1H-1,2,3-triazole (300 mg; 1.06 mmol; yield: 97%; green solid; UPLC purity: 96%) which is used in the consecutive step without purification.

### Example 48

### N-(4-Methanesulfonylpyridin-3-yl)-8-[3-(1H-1,2,3-triazol-4-yl)phenyl]quinoxalin-6-amine

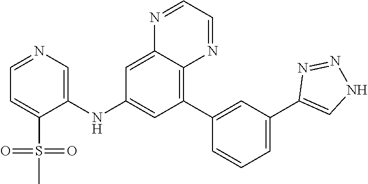 [SMILES: CS(=O)(=O)c1ccncc1Nc1cc(-c2cccc(-c3c[nH]nn3)c2)c2nccnc2c1]

A pressure vessel is charged with 8-chloro-N-(4-methanesulfonylpyridin-3-yl)quinoxalin-6-amine (Intermediate 3B, 80 mg; 0.22 mmol; 1 eq.), 4-[3-(tetramethyl-1,3,2-dioxaborolan-2-yl)phenyl]-1H-1,2,3-triazole (Intermediate 37, 91 mg; 0.32 mmol; 1.50 eq.), potassium carbonate (59 mg; 0.43 mmol; 2 eq.), dioxane (2 mL) and water (0.5 mL). The suspension is sparged with argon and Pd(dppf)Cl₂CH₂Cl₂ (18 mg; 0.02 mmol; 0.10 eq.) is added. The tube is sealed and the reaction mixture is stirred at 120° C. for 4 hours. Additional portions of 4-[3-(tetramethyl-1,3,2-dioxaborolan-2-yl)phenyl]-1H-1,2,3-triazole (5.5 eq.), Pd(dppf)Cl₂CH₂Cl₂ (0.15 eq.) and potassium carbonate (3 eq.) are added at this time and the reaction mixture is stirred at 120° C. an extra 15 hours. The mixture is filtered through a pad of Celite, rinsing the filter cake with EtOAc. Water is added to the filtrate, the layers are separated and the aqueous phase is extracted with EtOAc. The combined organic phases are washed with water, brine, dried over Na₂SO₄, filtered and concentrated. The residue is purified by FCC (EtOAc gradient in hexane) to afford N-(4-methanesulfonylpyridin-3-yl)-8-[3-(1H-1,2,3-triazol-4-yl)phenyl]quinoxalin-6-amine (45 mg; 0.10 mmol; yield 46%; yellow solid; HPLC purity: 97%).

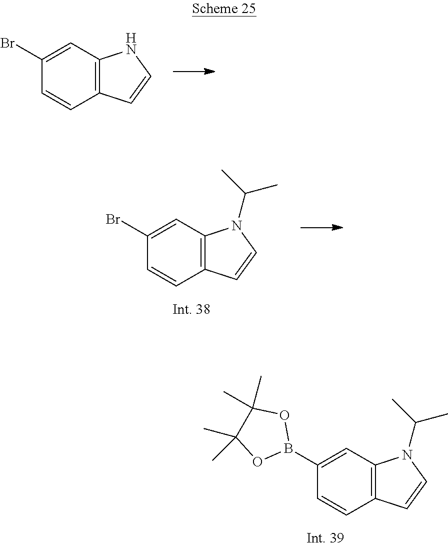 [SMILES: Brc1ccc2cc[nH]c2c1 ; CC(C)n1ccc2ccc(Br)cc21 ; CC(C)n1ccc2ccc(B3OC(C)(C)C(C)(C)O3)cc21]

### Intermediate 38

### 6-Bromo-1-(propan-2-yl)-1H-indole

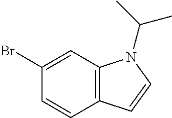

Sodium hydride (60% in mineral oil, 0.24 g; 6.12 mmol; 1.20 eq.) is added to an ice-bath cooled solution of 6-bromo-1H-indole (1 g; 5.10 mmol; 1 eq.) in anhydrous THF (10 mL) under argon. The mixture is left with stirring for 30 minutes and 2-iodopropane (0.66 mL; 6.63 mmol; 1.30 eq.) is added dropwise at 0° C. The mixture is allowed to slowly reach room temperature, the reaction flask is equipped with a condenser and the mixture is stirred at 60° C. under argon overnight. It is then poured onto ice and extracted with Et₂O/Hexane 1/1 (3 times). The combined organic layers are washed with water, brine, dried over Na₂SO₄, filtered and concentrated. The residue is filtered through a pad of silica gel, eluting with 4% EtOAc in hexane. The filtrate is concentrated to dryness to yield 6-bromo-1-(propan-2-yl)-1H-indole (1.1 g; 4.27 mmol; yield: 84%; light yellow oil; UPLC purity: 96%) which is used in the next step without further purification.

### Intermediate 39

### 1-(Propan-2-yl)-6-(tetramethyl-1,3,2-dioxaborolan-2-yl)-1H-indole

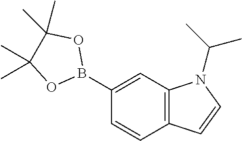 [SMILES: CC(C)n1ccc2ccc(B3OC(C)(C)C(C)(C)O3)cc21]

6-Bromo-1-(propan-2-yl)-1H-indole (Intermediate 38, 1 g; 4.03 mmol; 1 eq.), bis(pinacolato)diboron (1.33 g; 5.24 mmol; 1.30 eq.), dioxane (10 mL) and potassium acetate (0.79 g; 8.06 mmol; 2 eq.) are placed in a pressure vessel, the reaction mixture is sparged with argon and Pd(dppf)Cl₂ (30 mg; 0.04 mmol; 0.01 eq.) is added. The reaction tube is sealed and the mixture is stirred at 100° C. overnight. After cooling to room temperature, it is diluted with DCM and filtered over a pad of celite on top of a 2 cm layer of silica, eluting with DCM. The filtrate is concentrated to dryness, and the residue is purified by FCC (0-50% DCM gradient in hexane) to give 1-(propan-2-yl)-6-(tetramethyl-1,3,2-dioxaborolan-2-yl)-1H-indole (586 mg; 1.89 mmol; yield: 46.9%; white solid; UPLC purity: 92%) as a colorless oil which crystallizes on standing.

### Example 49

### N-(4-Methanesulfonylpyridin-3-yl)-8-[1-(propan-2-yl)-1H-indol-6-yl]quinoxalin-6-amine

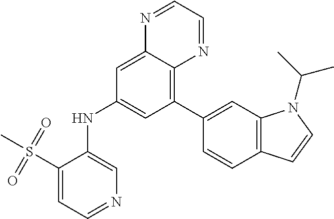 [SMILES: CC(C)n1ccc2ccc(-c3cc(Nc4cnccc4S(C)(=O)=O)cc4nccnc34)cc21]

Tetrakis(triphenylphosphine)palladium(0) (35 mg; 0.03 mmol; 0.1 eq.) is added to a degassed mixture of 8-chloro-N-(4-methanesulfonylpyridin-3-yl)quinoxalin-6-amine (100 mg; 0.30 mmol; 1 eq.), 1-(propan-2-yl)-6-(tetramethyl-1,3,2-dioxaborolan-2-yl)-1H-indole (102 mg; 0.36 mmol; 1.20 eq.), potassium carbonate (124 mg; 0.90 mmol; 3 eq.), dioxane (1 mL) and water (0.50 mL). The reaction mixture is stirred at 100° C. overnight and after coming back to room temperature, it is filtered through a pad of Celite on top of a 2 cm layer of silica, first eluting with DCM, then EtOAc and 10% MeOH in EtOAc to recover the product. The fractions containing the product are pooled and evaporated to dryness. The residue is purified by FCC (5% MeOH in EtOAc) to afford N-(4-methanesulfonylpyridin-3-yl)-8-[1-(propan-2-yl)-1H-indol-6-yl]quinoxalin-6-amine (99 mg; 0.21 mmol; yield: 71%; yellow powder; HPLC purity: 98%).

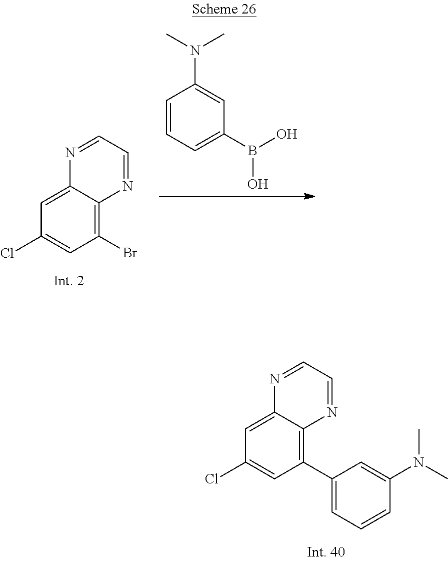

### Intermediate 40

### 3-(7-Chloroquinoxalin-5-yl)-N,N-dimethylaniline

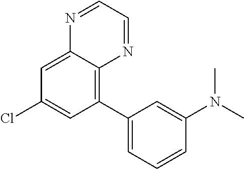 [SMILES: CN(C)c1cccc(-c2cc(Cl)cc3nccnc23)c1]

A pressure vessel is charged with 5-bromo-7-chloroquinoxaline (Intermediate 2, 300 mg; 1.23 mmol; 1 eq.), [3-(dimethylamino)phenyl]boronic acid (224 mg; 1.36 mmol; 1.10 eq.), DIPEA (0.43 mL; 2.46 mmol; 2 eq.), dioxane (3 mL) and water (3 mL). The suspension is sparged with argon and Pd(dppf)Cl₂ (90 mg; 0.12 mmol; 0.10 eq.) is added. The reaction tube is sealed and the mixture is stirred at 85° C. overnight. After coming back to room temperature, the mixture is filtered through a pad of Celite, rinsing the filter cake with DCM. Water is added to the filtrate. The layers are separated and the aqueous phase is extracted with DCM. The combined organic phases are washed with brine, dried over Na₂SO₄, filtered and concentrated in vacuo. The residue is purified by FCC (EtOAc gradient in hexane) to afford 3-(7-chloroquinoxalin-5-yl)-N,N-dimethylaniline (158 mg; 0.55 mmol; yield: 45%; yellow flakes; UPLC purity: 99%).

### Example 50

### 8-[3-(Dimethylamino)phenyl]-N-(4-methanesulfonylpyridin-3-yl)quinoxalin-6-amine

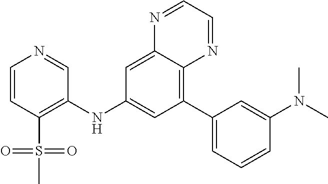 [SMILES: CN(C)c1cccc(-c2cc(Nc3cnccc3S(C)(=O)=O)cc3nccnc23)c1]

The title compound is prepared according to General Procedure 1 described in Example 1, with 3-(7-chloroquinoxalin-5-yl)-N,N-dimethylaniline (Intermediate 40, 120 mg; 0.42 mmol; 1 eq.), 4-methanesulfonylpyridin-3-amine hydrochloride (105 mg; 0.50 mmol; 1.20 eq.), BINAP (52 mg; 0.08 mmol; 0.20 eq.), cesium carbonate (683 mg; 2.10 mmol; 5 eq.), Pd(OAc)₂ (9 mg; 0.04 mmol; 0.10 eq.) in dioxane (4 mL). Conditions: 100° C. for 1 hour. Purification by FCC (EtOAc gradient in hexane, then 0-10% MeOH gradient in EtOAc) affords 8-[3-(dimethylamino)phenyl]-N-(4-methanesulfonylpyridin-3-yl)quinoxalin-6-amine (119 mg; 0.28 mmol; yield 66%; yellow powder; HPLC purity 97%).

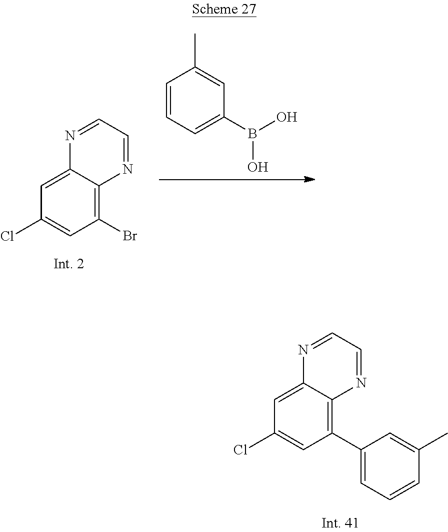

### Intermediate 41

### 7-Chloro-5-(3-methylphenyl)quinoxaline

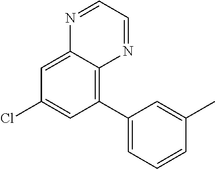 [SMILES: Cc1cccc(-c2cc(Cl)cc3nccnc23)c1]

A pressure vessel is charged with 5-bromo-7-chloroquinoxaline (Intermediate 1, 100 mg; 0.41 mmol; 1 eq.), m-tolylboronic acid (61 mg; 0.45 mmol; 1.10 eq.) and potassium carbonate (113 mg; 0.82 mmol; 2 eq.). Dioxane (2 mL) and water (1 mL) are added and the reaction mixture is sparged with argon for 5 minutes before adding Pd(PPh₃)₄ (24 mg; 0.02 mmol; 0.05 eq.). The reaction mixture is stirred at 80° C. for 6 h. After cooling to room temperature, it is partitioned between hexane and water. The aqueous phase is extracted with DCM and the combined organic phases are dried over anhydrous Na₂SO₄, filtered through a pad of celite and concentrated. The residue is purified by FCC (0-50% EtOAc gradient in hexane) to afford 7-chloro-5-(3-methylphenyl)quinoxaline (85 mg; 0.29 mmol; yield: 70%; white solid; UPLC purity: 86%).

### Example 51

### N-(4-Methanesulfonylpyridin-3-yl)-8-(3-methylphenyl)quinoxalin-6-amine

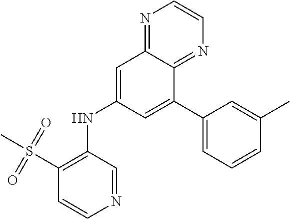

A pressure vessel is charged with 7-chloro-5-(3-methylphenyl)quinoxaline (Intermediate 41, 40 mg; 0.16 mmol; 1 eq.), 4-methanesulfonylpyridin-3-amine hydrochloride (36 mg; 0.17 mmol; 1.10 eq.), cesium carbonate (153 mg; 0.47 mmol; 3 eq.) and dioxane (3 mL). The mixture is sparged with argon for 5 minutes before BINAP (10 mg; 0.02 mmol; 0.10 eq.) and Pd(OAc)₂ (4 mg; 0.02 mmol; 0.10 eq.) are added. The reaction tube is sealed and the mixture is stirred at 150° C. for 1 h. After being cooled to room temperature, it is diluted with EtOAc, washed with water and brine, dried over Na₂SO₄, filtered, and concentrated. The residue is purified by FCC (0-100% EtOAc gradient in hexane, then 0-10% MeOH gradient in EtOAc) to afford N-(4-methanesulfonylpyridin-3-yl)-8-(3-methylphenyl)quinoxalin-6-amine (9.6 mg; 0.02 mmol; yield 16%; light green powder; HPLC purity: 99%).

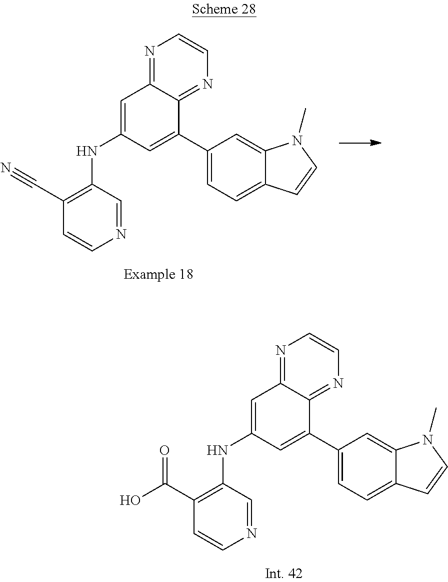 [SMILES: Cn1ccc2ccc(-c3cc(Nc4cnccc4C#N)cc4nccnc34)cc21 ; Cn1ccc2ccc(-c3cc(Nc4cnccc4C(=O)O)cc4nccnc34)cc21]

### Intermediate 42

### 3-{[8-(1-Methyl-1H-indol-6-yl)quinoxalin-6-yl]amino}pyridine-4-carboxylic acid

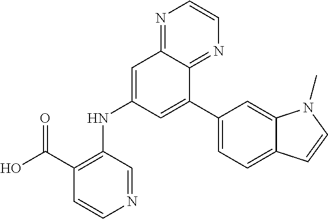 [SMILES: Cn1ccc2ccc(-c3cc(Nc4cnccc4C(=O)O)cc4nccnc34)cc21]

A round-bottom flask is charged with water (12 mL) and KOH (1.23 g; 21.98 mmol; 25 eq.) and the mixture is stirred till the hydroxide is completely dissolved. Then 3-{[8-(1-methyl-1H-indol-6-yl)quinoxalin-6-yl]amino}pyridine-4-carbonitrile (335 mg; 0.88 mmol; 1 eq.) and iPrOH (4 mL) are added and the mixture is stirred at 115° C. for 1 h. After coming back to room temperature; it is diluted with EtOAc, neutralized with 1M HCl and extracted with n-buthanol. The organic layer is dried with Na₂SO₄, filtered and concentrated in vacuo to afford 3-{[8-(1-methyl-1H-indol-6-yl)quinoxalin-6-yl]amino}pyridine-4-carboxylic acid (432 mg; 1.09 mmol; yield >100% UPLC purity: 99.7%).

### Example 52, General Procedure 13

### N-Methyl-3-{[8-(1-methyl-1H-indol-6-yl)quinoxalin-6-yl]amino}pyridine-4-carboxamide

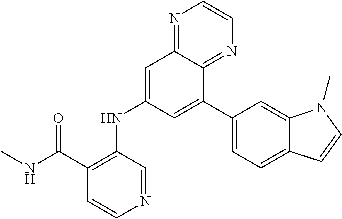 [SMILES: CNC(=O)c1ccncc1Nc1cc(-c2ccc3ccn(C)c3c2)c2nccnc2c1]

3-{[8-(1-Methyl-1H-indol-6-yl)quinoxalin-6-yl]amino}pyridine-4-carboxylic acid (70 mg; 0.18 mmol; 1 eq.), EDC.HCl (41 mg; 0.21 mmol; 1.20 eq.) and HOBt hydrate (33 mg; 0.21 mmol; 1.20 eq.) are dissolved in dioxane (7 mL) at room temperature. Triethylamine (0.11 mL; 0.88 mmol; 5 eq.) is added followed by methylamine hydrochloride (18 mg; 0.26 mmol; 1.50 eq.), after 30 minutes of stirring. After 24 hours at room temperature, UPLC-MS analysis shows 75% conversion and extra portions of N-(3-dimethylaminopropyl)-N′-ethylcarbodiimide hydrochloride (21 mg; 0.11 mmol; 0.60 eq.) and HOBt hydrate (16.5 mg; 0.11 mmol; 0.60 eq.) are added and the reaction is continued for another 24 h. Dioxane is then evaporated and the residue is vigorously stirred in a mixture of water and EtOAc. The medium is neutralized with 1M HCl and the phases are separated. The aqueous phase is extracted twice with EtOAc. The combined organic layers are washed with water and brine, dried over anhydrous Na₂SO₄, filtered, concentrated and purified by FCC (0-100% EtOAc gradient in hexane and continued with 0-10% MeOH gradient in EtOAc) to afford N-methyl-3-{[8-(1-methyl-1H-indol-6-yl)quinoxalin-6-yl]amino}pyridine-4-carboxamide (62 mg; 0.15 mmol; yield: 83%; yellow powder; HPLC purity: 97%).

### Example 53

### N,N-Dimethyl-3-{[8-(1-methyl-1H-indol-6-yl)quinoxalin-6-yl]amino}pyridine-4-carboxamide

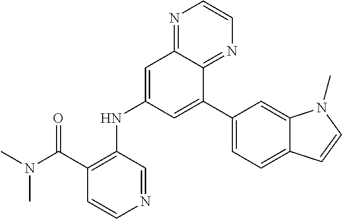 [SMILES: CN(C)C(=O)c1ccncc1Nc1cc(-c2ccc3ccn(C)c3c2)c2nccnc2c1]

Example 53 is prepared according to General Procedure 13 described in example 52, with 3-{[8-(1-methyl-1H-indol-6-yl)quinoxalin-6-yl]amino}pyridine-4-carboxylic acid (45 mg; 0.11 mmol; 1 eq.), EDC.HCl (27 mg; 0.14 mmol; 1.20 eq.), HOBt hydrate (21 mg; 0.14 mmol; 1.20 eq.), triethylamine (0.07 mL; 0.57 mmol; 5 eq.) and dimethylamine hydrochloride (14 mg; 0.17 mmol; 1.50 eq.) in dioxane (5 mL) at room temperature. Stirring is continued overnight at room temperature at which time new portions of N-(3-dimethylaminopropyl)-N′-ethylcarbodiimide hydrochloride (13 mg; 0.07 mmol; 0.60 eq.) and HOBt hydrate (11 mg; 0.07 mmol; 0.60 eq.) are added. Stirring at room temperature is continued for 24 h. Purification by FCC (column: PF—NH2/30 um/6G, 0-100% EtOAc gradient: in hexane) affords N,N-dimethyl-3-{[8-(1-methyl-1H-indol-6-yl)quinoxalin-6-yl]amino}pyridine-4-carboxamide (31 mg; 0.07 mmol; yield: 62%; yellow powder; HPLC purity: 96.8%).

### Example 54

### 3-{[8-(1-Methyl-1H-indol-6-yl)quinoxalin-6-yl]amino}-N-(pyrimidin-5-yl)pyridine-4-carboxamide

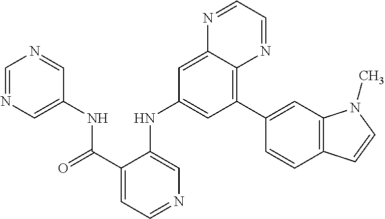 [SMILES: Cn1ccc2ccc(-c3cc(Nc4cnccc4C(=O)Nc4cncnc4)cc4nccnc34)cc21]

A pressure vessel is charged with 3-{[8-(1-methyl-1H-indol-6-yl)quinoxalin-6-yl]amino}pyridine-4-carboxamide (Example 19, 13 mg; 0.03 mmol; 1 eq.), potassium phosphate tribasic (8 mg; 0.04 mmol; 1.20 eq.), a solution of 5-bromo-pyrimidine (8 mg; 0.05 mmol; 1.50 eq.) in t-butanol (1 mL) and Me₄tBuXPhos (4 mg; 0.01 mmol; 0.25 eq.). The mixture is sparged with argon for 5 minutes and Pd₂(dba)₃ (2 mg; 1.5 μmol; 0.05 eq.) is added. The vessel is sealed and reaction mixture is stirred at 110° C. for 24 h. After coming back to room temperature, it is filtered through a Celite pad, rinsing the filter cake with EtOAc. Water is added to the filtrate under stirring, the phases are separated and the aqueous layer is extracted with EtOAc. The combined organic phases are washed with brine, dried over Na₂SO₄. The solvent is evaporated and the residue is purified by FCC (column: PF—NH₂/30 μm/6G, 30-100% EtOAc gradient in hexane followed by 0-10% MeOH gradient in EtOAc) to yield 3-{[8-(1-methyl-1H-indol-6-yl)quinoxalin-6-yl]amino}-N-(pyrimidin-5-yl)pyridine-4-carboxamide (12 mg; 0.03 mmol; yield: 77%; yellow powder; HPLC purity 99.5%).

### Example 55

### 3-{[8-(1-Methyl-1H-indol-6-yl)quinoxalin-6-yl]amino}-N-(pyrimidin-5-ylmethyl)pyridine-4-carboxamide

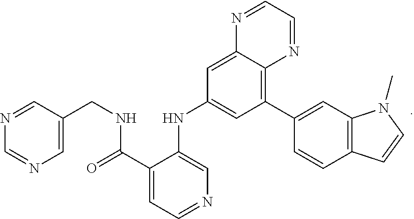 [SMILES: Cn1ccc2ccc(-c3cc(Nc4cnccc4C(=O)NCc4cncnc4)cc4nccnc34)cc21]

The title compound is prepared according to General Procedure 13, described in example 52, with 3-{[8-(1-methyl-1H-indol-6-yl)quinoxalin-6-yl]amino}pyridine-4-carboxylic acid (70 mg; 0.18 mmol; 1 eq.), EDC.HCl (41 mg; 0.21 mmol; 1.20 eq.), HOBt hydrate (33 mg; 0.21 mmol; 1.20 eq.), triethylamine (0.11 mL; 0.88 mmol; 5 eq.) and pyrimidin-5-ylmethanamine (30 mg; 0.26 mmol; 1.5 eq.) in dioxane (7 mL) at room temperature. After 24 h at room temperature, extra portions of EDC.HCl (21 mg; 0.11 mmol; 0.60 eq.) and HOBt hydrate (16 mg; 0.11 mmol; 0.60 eq.) are added. Stirring is continued for 24 h at room temperature. Workup as in example 52. Purification by FCC (column: PF—NH2/30 um/6G, 0-100% EtOAc gradient in hexane followed by 0-10% MeOH gradient in EtOAc) affords 3-{[8-(1-methyl-1H-indol-6-yl)quinoxalin-6-yl]amino}-N-(pyrimidin-5-ylmethyl)pyridine-4-carboxamide (74 mg; 0.15 mmol; yield: 84%; yellow powder; HPLC purify: 98.1%).

### Example 56

### 3-{[8-(1-Methyl-1H-indol-6-yl)quinoxalin-6-yl]amino}-N-[(1-methyl-1H-pyrazol-4-yl)methyl]pyridine-4-carboxamide

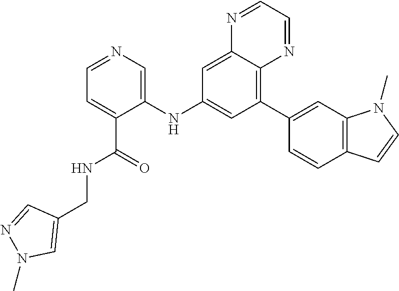 [SMILES: Cn1cc(CNC(=O)c2ccncc2Nc2cc(-c3ccc4ccn(C)c4c3)c3nccnc3c2)cn1]

Example 56 is prepared according to General Procedure 13, described in example 52, with 3-{[8-(1-methyl-1H-indol-6-yl)quinoxalin-6-yl]amino}pyridine-4-carboxylic acid (70 mg; 0.18 mmol; 1 eq.), EDC.HCl (41 mg; 0.21 mmol; 1.20 eq.), HOBt hydrate (33 mg; 0.21 mmol; 1.20 eq.), triethylamine (0.11 mL; 0.88 mmol; 5 eq.) and (1-methyl-1H-pyrazol-4-yl)methylamine (29 mg; 0.26 mmol; 1.5 eq.) in dioxane (7 mL) at room temperature. After 24 h of stirring at room temperature, extra portions of EDC.HCl (21 mg; 0.11 mmol; 0.60 eq.) and HOBt hydrate (16.5 mg; 0.11 mmol; 0.60 eq.) are added. Stirring is continued for 24 h at room temperature. Workup as in example 52. Purification by FCC (column: PF—NH2/30 um/6G, 0-100% EtOAc gradient in hexane followed by 0-10% MeOH gradient in EtOAc) afforded 3-{[8-(1-methyl-1H-indol-6-yl)quinoxalin-6-yl]amino}-N-[(1-methyl-1H-pyrazol-4-yl)methyl]pyridine-4-carboxamide (77 mg; 0.15 mmol; yield: 85%; yellow powder; HPLC purity: 95%).

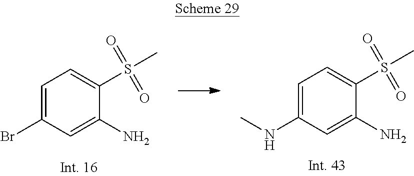

### Intermediate 43

### 4-Methanesulfonyl-N1-methylbenzene-1,3-diamine

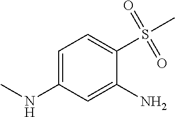

A pressure vessel is charged with 5-bromo-2-methanesulfonylaniline (140 mg; 0.56 mmol; 1 eq.), methylamine (40% solution in water, 0.48 mL; 5.60 mmol; 10 eq.) and DMSO (1 mL). The tube is sealed and the mixture is stirred at 130° C. overnight. Another portion of methylamine (40% solution in water, 0.48 mL; 5.60 mmol; 10 eq.) is added and stirring at 130° C. is continued for 48 hrs. The reaction mixture is cooled to room temperature and partitioned between EtOAc and water. The organic layer is washed with brine, dried over Na₂SO₄ and concentrated. The residue is purified by FCC (5-40% EtOAc gradient in hexane) to afford 4-methanesulfonyl-N1-methylbenzene-1,3-diamine (20 mg; 0.10 mmol; yield: 17%; light yellow oil; UPLC purity: 97%).

### Example 57

### 4-Methanesulfonyl-N1-methyl-N3-[8-(1-methyl-1H-indol-6-yl)quinoxalin-6-yl]benzene-1,3-diamine

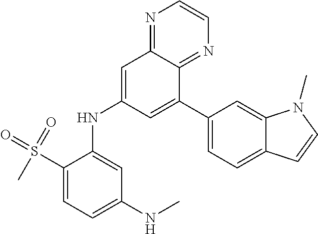 [SMILES: CNc1ccc(S(C)(=O)=O)c(Nc2cc(-c3ccc4ccn(C)c4c3)c3nccnc3c2)c1]

The title compound is prepared according to General Procedure 1 described in Example 1, with 7-chloro-5-(1-methyl-1H-indol-6-yl)quinoxaline (25 mg; 0.08 mmol; 1 eq.), 4-methanesulfonyl-N1-methylbenzene-1,3-diamine (21 mg; 0.10 mmol; 1.2 eq.), cesium carbonate (39 mg; 0.12 mmol; 1.40 eq.), BINAP (11 mg; 0.02 mmol; 0.20 eq.) and Pd(OAc)₂ (4 mg; 0.02 mmol; 0.20 eq.) in dioxane (1 mL). Conditions: 130° C. for 3 hours. Purification by FCC (0-20% MeOH gradient in EtOAc) to afford 4-methanesulfonyl-N1-methyl-N3-[8-(1-methyl-1H-indol-6-yl)quinoxalin-6-yl]benzene-1,3-diamine (23 mg; 0.05 mmol; yield: 57%; yellow powder; HPLC purity: 94.8%).

### Example 58

### 8-[3-(Chloromethyl)-1-benzofuran-5-yl]-N-(4-methanesulfonylpyridin-3-yl)quinoxalin-6-amine

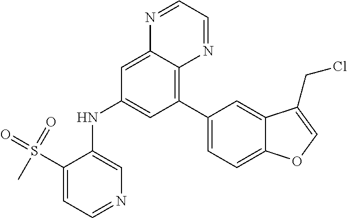 [SMILES: CS(=O)(=O)c1ccncc1Nc1cc(-c2ccc3occ(CCl)c3c2)c2nccnc2c1]

(4-Methanesulfonylpyridin-3-yl)-[8-(3-methyl-benzofuran-5-yl)-quinoxalin-6-yl]-amine (Example 22, 23 mg; 0.05 mmol; 1 eq.) is dissolved in CHCl₃ (690 μl) and by Palau'chlor (13 mg; 62 μmol; 1.2 eq.) is added in one portion. The reaction mixture is stirred at room temperature overnight. The solvent is then evaporated, the residue is dissolved in DCM and the resulting solution is washed with water and brine. The organic phase is dried over sodium sulfate filtered and evaporated in vacuo. The crude product is purified by FCC (EtOAc gradient in hexane) to afford [8-(3-Chloromethyl-benzofuran-5-yl)-quinoxalin-6-yl]-(4-methanesulfonylpyridin-3-yl)-amine (10 mg; 0.02 mmol; 35%; light yellow fine powder; HPLC purity: 85.6%).

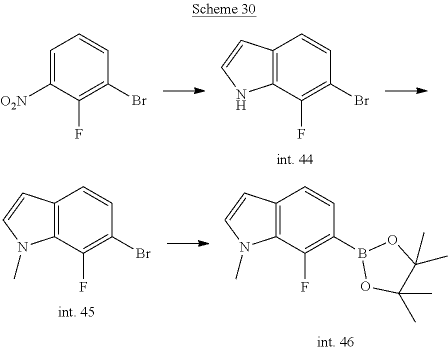 [SMILES: O=[N+]([O-])c1cccc(Br)c1F ; Fc1c(Br)ccc2cc[nH]c12 ; Cn1ccc2ccc(Br)c(F)c21 ; Cn1ccc2ccc(B3OC(C)(C)C(C)(C)O3)c(F)c21]

### Intermediate 44

### 6-Bromo-7-fluoro-1H-indole

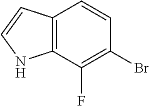 [SMILES: Fc1c(Br)ccc2cc[nH]c12]

Vinyl magnesium bromide (29 mL; 29.09 mmol; 3.2 eq.) is rapidly added to a solution of 1-bromo-2-fluoro-3-nitrobenzene (2 g; 9.09 mmol; 1 eq.) in anhydrous THF (20 mL) at −70° C. and the mixture is stirred at −40° C. for 1 h. The reaction is quenched with sat. aqueous ammonium chloride and extracted twice with EtOAc. The combined organic layers are washed with brine, dried over sodium sulfate and evaporated. The residue is purified by FCC (10% DCM in hexane) to afford 6-bromo-7-fluoro-1H-indole (222 mg;

0.93 mmol; 10%; yellow wax; UPLC purity: 90%).

### Intermediate 45, General Procedure 14

### 6-Bromo-7-fluoro-1-methyl-1H-indole

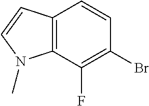

Sodium hydride (60% in mineral oil, 0.08 g; 2.06 mmol; 2 eq.) is added to an ice-bath cooled solution of 6-bromo-7-fluoro-1H-indole (Intermediate 44, 220 mg; 1.03 mmol; 1 eq.) in anhydrous THF (2.5 mL) under argon. The mixture is left with stirring for 30 minutes at 0° C. and iodomethane (0.08 mL; 1.34 mmol; 1.3 eq.) is added dropwise. The mixture is left with stirring at room temperature under argon overnight. It is then partitioned between water and EtOAc and the aqueous phase is extracted with EtOAc. The combined organic layers are washed with brine, dried over sodium sulfate, filtered and evaporated to dryness. The residue is purified by FCC (10% DCM in hexane) to afford 6-bromo-7-fluoro-1-methyl-1H-indole (180 mg; 0.73 mmol; 71%; in color oil; UPLC purity: 92%).

### Intermediate 46

### 7-Fluoro-1-methyl-6-(tetramethyl-1,3,2-dioxaborolan-2-yl)-1H-indole

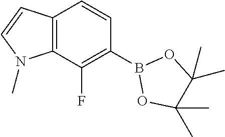 [SMILES: Cn1ccc2ccc(B3OC(C)(C)C(C)(C)O3)c(F)c21]

The title compound is prepared according to General Procedure 8 described for Intermediate 14, using 6-bromo-7-fluoro-1-methyl-1H-indole (Intermediate 44, 160 mg; 0.70 mmol; 1 eq.), bis(pinacolato)diboron (230 mg; 0.91 mmol; 1.3 eq.), potassium acetate (0.14 g; 1.40 mmol; 2 eq.) and Pd(dppf)Cl₂ (5 mg; 0.01 mmol; 0.01 eq.). Purification by FCC (0% to 30% DCM gradient in hexane) affords 7-fluoro-1-methyl-6-(tetramethyl-1,3,2-dioxaborolan-2-yl)-1H-indole (105 mg; 0.36 mmol; 52%; off-white solid; UPLC purity: 95%).

### Example 59

### 8-(7-Fluoro-1-methyl-1H-indol-6-yl)-N-(4-methanesulfonylpyridin-3-yl)quinoxalin-6-amine

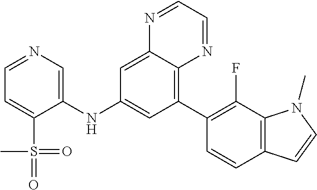 [SMILES: Cn1ccc2ccc(-c3cc(Nc4cnccc4S(C)(=O)=O)cc4nccnc34)c(F)c21]

Tetrakis(triphenylphosphine)palladium(0) (26 mg; 0.02 mmol; 0.1 eq.) is added to a deaerated mixture of (8-Chloro-quinoxalin-6-yl)-(4-methanesulfonylpyridin-3-yl)-amine (Intermediate 3B, 75 mg; 0.22 mmol; 1 eq.), 7-fluoro-1-methyl-6-(4,4,5,5-tetramethyl-[1,3,2]dioxaborolan-2-yl)-1H-indole (Intermediate 45, 78 mg; 0.27 mmol; 1.2 eq.), potassium carbonate (93 mg; 0.67 mmol; 3 eq.) in dioxane (2 mL) and water (1 mL) in a microwave vial. The vial is sealed and the reaction mixture is stirred at 100° C. overnight. After coming back to room temperature, it is filtered through a pad of celite, eluting with DCM. The resulting solution is washed with water, dried over sodium sulfate and evaporated under reduced pressure. The title compound is purified by three consecutive FCCs, the first using 2% MeOH in DCM, the second using 2% MeOH in EtOAc, and the third using 10% acetone in DCM as eluents, to afford 8-(7-fluoro-1-methyl-1H-indol-6-yl)-N-(4-methanesulfonylpyridin-3-yl)quinoxalin-6-amine (9 mg; 0.02 mmol; 9%; yellow powder; HPLC purity: 96.4%).

### Example 60, General Procedure 15

### 8-(4-Ethylphenyl)-N-(4-methanesulfonylpyridin-3-yl)quinoxalin-6-amine

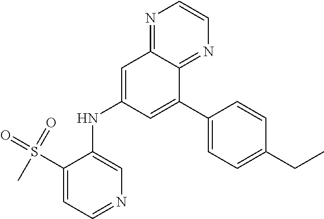 [SMILES: CCc1ccc(-c2cc(Nc3cnccc3S(C)(=O)=O)cc3nccnc23)cc1]

Tetrakis(triphenylphosphine)palladium(0) (26 mg; 0.02 mmol; 0.1 eq.) is added to a deaerated mixture of 8-chloro-N-(4-methanesulfonylpyridin-3-yl)quinoxalin-6-amine (Intermediate 3B, 75 mg; 0.22 mmol; 1 eq.), (4-ethylphenyl)boronic acid (40 mg; 0.27 mmol; 1.2 eq.), potassium carbonate (93 mg; 0.67 mmol; 3 eq.) in dioxane (2 mL) and water (1 mL) in a microwave vial. The vial is sealed and the reaction mixture is stirred at 100° C. overnight. After coming back to room temperature, it is filtered through a pad of celite, eluting with DCM. The resulting solution is washed with water, dried over sodium sulfate, and evaporated under reduced pressure. The residue is purified by two consecutive FCC, the first using 1% MeOH in DCM, the second using 10% acetone in DCM, to afford 8-(4-ethylphenyl)-N-(4-methanesulfonylpyridin-3-yl)quinoxalin-6-amine (11 mg; 0.03 mmol; 12%; off-white solid; HPLC purity: 99.9%).

### Example 61

### 8-(1H-1,3-Benzodiazol-5-yl)-N-(4-methanesulfonylpyridin-3-yl)quinoxalin-6-amine

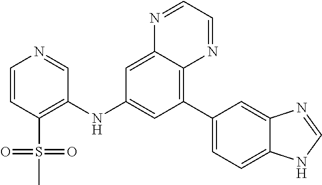 [SMILES: CS(=O)(=O)c1ccncc1Nc1cc(-c2ccc3[nH]cnc3c2)c2nccnc2c1]

The title compound is prepared according to General Procedure 15 described in Example 60, using 8-chloro-N-(4-methanesulfonylpyridin-3-yl)quinoxalin-6-amine (Intermediate 3B, 75 mg; 0.22 mmol; 1 eq.), 5-(4,4,5,5-tetramethyl-[1,3,2]dioxaborolan-2-yl)-1H-benzoimidazole (66 mg; 0.27 mmol; 1.2 eq.), potassium carbonate (93 mg; 0.67 mmol; 3 eq.), tetrakis(triphenylphosphine)-palladium(0) (26 mg; 0.02 mmol; 0.10 eq.) and THF as extraction solvent. Conditions: 100° C., overnight. Purification by FCC (0% to 40% MeOH gradient in EtOAc) affords 8-(1H-1,3-benzodiazol-5-yl)-N-(4-methanesulfonylpyridin-3-yl)quinoxalin-6-amine (5 mg; 0.01 mmol; 5%; yellow powder; HPLC purity: 97.1%).

### Example 62, General Procedure 16

### N-(4-Methanesulfonylpyridin-3-yl)-8-(3-methoxyphenyl)quinoxalin-6-amine

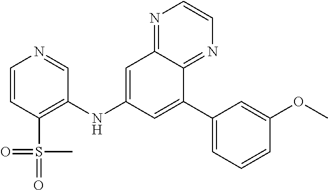 [SMILES: COc1cccc(-c2cc(Nc3cnccc3S(C)(=O)=O)cc3nccnc23)c1]

A pressure vessel is charged with chloro-N-(4-methanesulfonylpyridin-3-yl)quinoxalin-6-amine (Intermediate 3B, 52 mg; 0.16 mmol; 1 eq.), (3-methoxyphenyl)boronic acid (50 mg; 0.31 mmol; 2 eq.), cesium carbonate (153 mg; 0.47 mmol; 3 eq.) in dioxane (1 mL) and water (0.5 mL). The reaction mixture is sparged with argon under sonication and tetrakis(triphenylphosphine)palladium(0) (27 mg; 0.02 mmol; 0.15 eq.) is added. The reaction mixture is stirred at 100° C. overnight before being cooled to room temperature and partitioned between EtOAc and water. The aqueous phase is extracted with EtOAc and the combined organic layers are washed with water and brine, dried over sodium sulfate, filtered and concentrated in vacuo. The residue is purified by FCC (0% to 100% EtOAc gradient in hexane followed by 0% to 3% MeOH gradient in EtOAc) to afford N-(4-methanesulfonylpyridin-3-yl)-8-(3-methoxyphenyl)quinoxalin-6-amine (54 mg; 0.13 mmol; 85%; pale yellow powder; HPLC purity: 98.9%).

### Example 63

### 8-(3,3-Dimethyl-2,3-dihydro-1-benzofuran-5-yl)-N-(4-methanesulfonylpyridin-3-yl)quinoxalin-6-amine

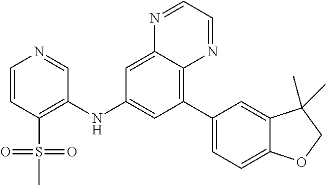 [SMILES: CC1(C)COc2ccc(-c3cc(Nc4cnccc4S(C)(=O)=O)cc4nccnc34)cc21]

The title compound is prepared according to General Procedure 16 described in Example 62, using chloro-N-(4-methanesulfonylpyridin-3-yl)quinoxalin-6-amine (Intermediate 3B, 45 mg; 0.13 mmol; 1 eq.), 3,3-dimethyl-5-(4,4,5,5-tetramethyl-[1,3,2]dioxaborolan-2-yl)-2,3-dihydrobenzofuran (60 mg; 0.22 mmol; 1.6 eq.), cesium carbonate (133 mg; 0.40 mmol; 3 eq.) and tetrakis(triphenylphosphine)palladium(0) (23 mg; 0.02 mmol; 0.15 eq.). Purification by FCC (0% to 100% EtOAc gradient in hexane) affords 8-(3,3-dimethyl-2,3-dihydro-1-benzofuran-5-yl)-N-(4-methanesulfonylpyridin-3-yl)quinoxalin-6-amine (59 mg; 0.13 mmol; 95%; pale yellow powder; HPLC purity: 96.6%).

### Example 65

### 8-(3-Ethylphenyl)-N-(4-methanesulfonylpyridin-3-yl)quinoxalin-6-amine

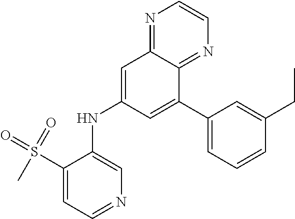 [SMILES: CCc1cccc(-c2cc(Nc3cnccc3S(C)(=O)=O)cc3nccnc23)c1]

The title compound is prepared according to General Procedure 15 described in Example 60, using chloro-N-(4-methanesulfonylpyridin-3-yl)quinoxalin-6-amine (Intermediate 3B, 75 mg; 0.22 mmol; 1 eq.), (3-ethylphenyl)boronic acid (40 mg; 0.27 mmol; 1.2 eq.), cesium carbonate (212 mg; 0.67 mmol; 3 eq.) and tetrakis(triphenylphosphine)palladium(0) (26 mg; 0.02 mmol; 0.1 eq.). Conditions: 100° C., overnight. Purification by FCC (50% to 75% EtOAc gradient in hexane) affords 8-(3-ethylphenyl)-N-(4-methanesulfonylpyridin-3-yl)quinoxalin-6-amine (84 mg; 0.21 mmol; 93%; white powder; HPLC purity: 99.9%).

### Example 66, General Procedure 17

### 8-(2-Amino-5-methylphenyl)-N-(4-methanesulfonylpyridin-3-yl)quinoxalin-6-amine

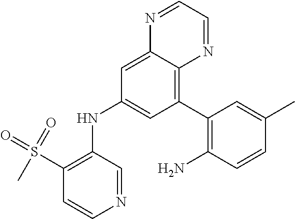 [SMILES: Cc1ccc(N)c(-c2cc(Nc3cnccc3S(C)(=O)=O)cc3nccnc23)c1]

A 5-mL microwave vial is charged with sodium carbonate (115 mg; 1.09 mmol; 5 eq.), 4-methyl-2-(4,4,5,5-tetramethyl-[1,3,2]dioxaborolan-2-yl)-phenylamine (56 mg; 0.24 mmol; 1.10 eq.), chloro-N-(4-methanesulfonylpyridin-3-yl)quinoxalin-6-amine (Intermediate 3B, 75 mg; 0.22 mmol; 1 eq.), followed by water (0.5 mL), ethanol (0.50 mL) and toluene (1 mL). The mixture is sparged with argon under sonication, and tetrakis(triphenylphosphine)palladium(0) (13 mg; 0.01 mmol; 0.05 eq.) is added. The vial is sealed and the mixture is stirred at 110° C. overnight. After coming back to room temperature, the reaction mixture is filtered through a pad of celite, eluting with DCM. The filtrate is washed with water, dried over sodium sulfate, filtered through a 1-cm high pad of neutral alumina (rinsing the filter cake with EtOAc), and evaporated in vacuo. The residue is purified by FCC (100% EtOAc) to afford 8-(2-amino-5-methylphenyl)-N-(4-methanesulfonylpyridin-3-yl)quinoxalin-6-amine (38 mg; 0.09 mmol; 43%; yellow powder; HPLC purity: 99.7%).

### Example 67

### 2-{7-[(4-Methanesulfonylpyridin-3-yl)amino]quinoxalin-5-yl}-4-methylphenol

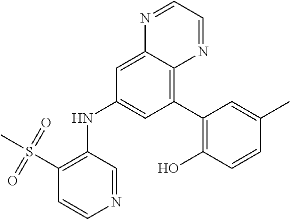

The title compound is prepared according to General Procedure 17 described in Example 66, using sodium carbonate (115 mg; 1.09 mmol; 5 eq.), (2-hydroxy-5-methyl-phenyl)boronic acid (36 mg; 0.24 mmol; 1.1 eq.), chloro-N-(4-methanesulfonylpyridin-3-yl)quinoxalin-6-amine (Intermediate 3B, 75 mg; 0.22 mmol; 1 eq.) and tetrakis(triphenylphosphine)palladium(0) (13 mg; 0.01 mmol; 0.05 eq.). Conditions: 100° C., overnight. Purification by FCC (100% EtOAc) to afford 2-{7-[(4-methanesulfonylpyridin-3-yl)amino]quinoxalin-5-yl}-4-methylphenol (48 mg; 0.12 mmol; 53%; yellow powder; HPLC purity: 98.4%).

### Example 68

### 8-(1-Methyl-1H-indol-6-yl)-N-[4-(1H-1,2,3,4-tetrazol-5-yl)pyridin-3-yl]quinoxalin-6-amine

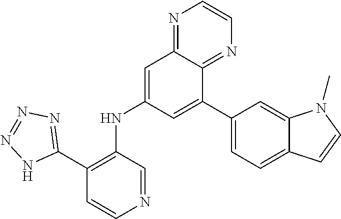 [SMILES: Cn1ccc2ccc(-c3cc(Nc4cnccc4-c4nnn[nH]4)cc4nccnc34)cc21]

A mixture of 3-{[8-(1-methyl-1H-indol-6-yl)quinoxalin-6-yl]amino}pyridine-4-carbonitrile (Example 18, 45 mg; 0.12 mmol; 1 eq.), sodium azide (23 mg; 0.35 mmol; 3 eq.) and triethylamine hydrochloride (49 mg; 0.35 mmol; 3 eq.), in anhydrous toluene (5 mL) and a few drops of DMF, is heated in a pressure vessel at 110° C. under argon for 20 h. Additional portions of sodium azide (11 mg; 0.18 mmol; 1.5 eq.) and triethylamine hydrochloride (24 mg; 0.18 mmol; 1.5 eq.) are added and the reaction is further heated for 12 h. The reaction mixture is cooled to room temperature, the toluene is evaporated off and the residue is triturated in toluene (1 mL). It is dissolved in EtOAc, water is added and the mixture is neutralized with 1M HCl. The organic layer is washed with water and brine, dried over anhydrous Na₂SO₄ and filtered through a pad of celite. The filtrate is concentrated in vacuo to give 8-(1-methyl-1H-indol-6-yl)-N-[4-(1H-1,2,3,4-tetrazol-5-yl)pyridin-3-yl]quinoxalin-6-amine (37 mg; 0.09 mmol; 74%; olive green powder; HPLC purity: 98.1%).

### Example 69

### N-(4-Chloropyridin-3-yl)-8-(1-methyl-1H-indol-6-yl)quinoxalin-6-amine

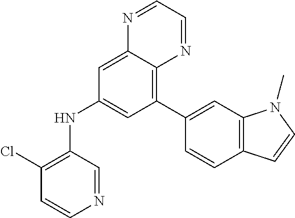 [SMILES: Cn1ccc2ccc(-c3cc(Nc4cnccc4Cl)cc4nccnc34)cc21]

The title compound is prepared according to General Procedure 5 described in Example 8, using 7-chloro-5-(1-methyl-1H-indol-6-yl)quinoxaline (Intermediate 2B, 100 mg; 0.34 mmol; 1 eq.), 4-chloropyridin-3-ylamine (87 mg; 0.67 mmol; 2 eq.), K₂CO₃ (186 mg; 1.35 mmol; 4 eq.), BippyPhos (34 mg; 0.07 mmol; 0.2 eq.) and bis[cinnamyl palladium(II) (7 mg; 0.01 mmol; 0.04 eq.). Conditions: 120° C. 12 hours. Purification by FCC (SiO₂ column washed with 1% Et₃N/DCM before purification, EtOAc gradient in hexane), to afford N-(4-chloropyridin-3-yl)-8-(1-methyl-1H-indol-6-yl)quinoxalin-6-amine (6 mg; 0.01 mmol; 4%; yellow powder; HPLC purity: 91.9%).

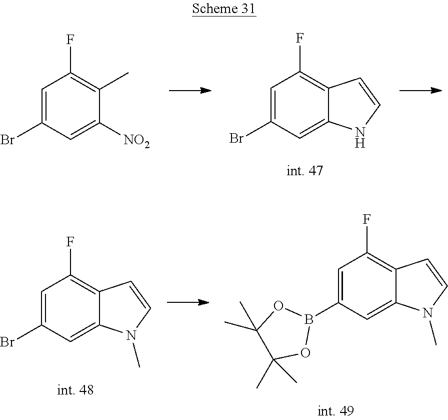 [SMILES: Cc1c(F)cc(Br)cc1[N+](=O)[O-] ; Fc1cc(Br)cc2[nH]ccc12 ; Cn1ccc2c(F)cc(Br)cc21 ; Cn1ccc2c(F)cc(B3OC(C)(C)C(C)(C)O3)cc21]

### Intermediate 47

### 6-Bromo-4-fluoro-1H-indole

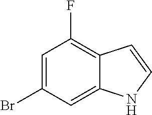 [SMILES: Fc1cc(Br)cc2[nH]ccc12]

A dry 100-mL round bottom flask is charged with 4-bromo-2-fluoro-6-nitrotoluene (1.3 g; 5.56 mmol; 1 eq.), N,N-dimethylformamide diisopropyl acetal (2.6 mL; 12.22 mmol; 2.2 eq.), triethylamine (0.85 mL; 6.11 mmol; 1.1 eq.), anhydrous DMF (5 mL) and the mixture is stirred at 130° C. for 2 h. After removal of the solvent, the residue is dissolved in a mixture of toluene (30 mL) and acetic acid (40 mL), followed by the addition of iron (6.2 g; 111.10 mmol; 20 eq.) and silica (6 g). The dark red mixture is heated to 100° C. with vigorous stirring. for 30 min. The mixture is then cooled to room temperature, diluted with EtOAc, filtered and the solids are thoroughly washed with EtOAc. The combined filtrates are washed with sat. aq. Na₂S₂O₅, sat. aq. NaHCO₃ and brine, dried over Na₂SO₄ and concentrated in vacuo. The residue is purified by FCC (20% DCM in hexanes) to afford 6-bromo-4-fluoro-1H-indole (814 mg; 3.75 mmol; 68%; UPLC purity: 99%).

### Intermediate 48

### 6-Bromo-4-fluoro-1-methyl-1H-indole

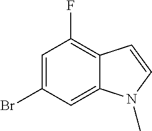

The title compound is prepared according to General Procedure 14 described for Intermediate 45, using 6-bromo-4-fluoro-1H-indole (Intermediate 47, 300 mg; 1.39 mmol; 1 eq.), sodium hydride (60% in mineral oil, 111 mg; 2.78 mmol; 2 eq.) and iodomethane (0.11 mL; 1.80 mmol; 1.3 eq.) in anhydrous THF (3 mL). The obtained crude 6-bromo-4-fluoro-1-methyl-1H-indole (315 mg; 1.35 mmol; 97%; UPLC purity: 98%) is used in the next step without further purification.

### Intermediate 49

### 4-Fluoro-1-methyl-6-(tetramethyl-1,3,2-dioxaborolan-2-yl)-1H-indole

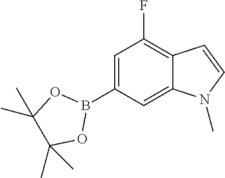 [SMILES: Cn1ccc2c(F)cc(B3OC(C)(C)C(C)(C)O3)cc21]

The title compound is prepared according to General Procedure 8 described for Intermediate 14, using 6-bromo-4-fluoro-1-methyl-1H-indole (Intermediate 48, 310 mg; 1.36 mmol; 1 eq.), bis(pinacolato)diboron (449 mg; 1.77 mmol; 1.3 eq.), potassium acetate (267 mg; 2.72 mmol; 2 eq) and Pd(dppf)Cl₂ (10 mg; 0.01 mmol; 0.01 eq.). Purification by FCC (0% to 1% MeOH gradient in DCM) to afford 4-fluoro-1-methyl-6-(tetramethyl-1,3,2-dioxaborolan-2-yl)-1H-indole (300 mg; 0.86 mmol; 63%; beige solid; UPLC purity: 79%).

### Example 71

### 8-(4-Fluoro-1-methyl-1H-indol-6-yl)-N-(4-methanesulfonylpyridin-3-yl)quinoxalin-6-amine (71)

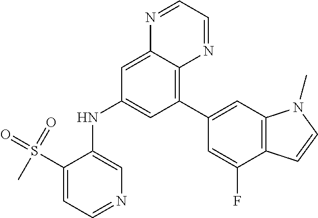 [SMILES: Cn1ccc2c(F)cc(-c3cc(Nc4cnccc4S(C)(=O)=O)cc4nccnc34)cc21]

The title compounds are prepared according to General Procedure 16 described in Example 62, using chloro-N-(4-methanesulfonylpyridin-3-yl)quinoxalin-6-amine (Intermediate 3B, 75 mg; 0.22 mmol; 1 eq.), 4-fluoro-1-methyl-6-(4,4,5,5-tetramethyl-[1,3,2]dioxaborolan-2-yl)-1H-indole (94 mg; 0.27 mmol; 1.2 eq.), cesium carbonate (221 mg; 0.67 mmol; 3 eq.) and tetrakis(triphenylphosphine)palladium(0) (26 mg; 0.02 mmol; 0.1 eq.) in dioxane (2 mL) and water (1 mL). Conditions: 130° C. under microwave irradiation for 45 min. Purification by FCC (0% to 50% EtOAc gradient in hexane) affords 8-(4-fluoro-1-methyl-1H-indol-6-yl)-N-(4-methanesulfonyl-pyridin-3-yl)quinoxalin-6-amine (52 mg; 0.11 mmol; 25%; yellow powder; HPLC purity: 96.8%).

### Example 72

### 4-Methanesulfonyl-3-{[8-(3-methyl-1-benzofuran-5-yl)quinoxalin-6-yl]amino}pyridin-1-ium-1-olate

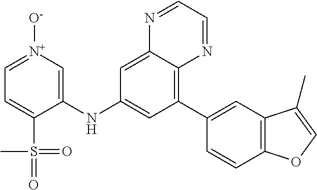 [SMILES: Cc1coc2ccc(-c3cc(Nc4c[n+]([O-])ccc4S(C)(=O)=O)cc4nccnc34)cc12]

The title compound is prepared according to General Procedure 1 described in Example 1, using 3-bromo-4-methanesulfonylpyridine 1-oxide (Intermediate 32, 64 mg; 0.23 mmol; 1.1 eq.), 8-(3-methyl-benzofuran-5-yl)-quinoxalin-6-ylamine (Example 80, 60 mg; 0.21 mmol; 1 eq.), cesium carbonate (170 mg; 0.52 mmol; 2.5 eq.), BINAP (26 mg; 0.04 mmol; 0.2 eq.) and palladium(II) acetate (10 mg; 0.04 mmol; 0.2 eq.). Purification by FCC (0% to 20% MeOH gradient in EtOAc) to afford 4-methanesulfonyl-3-{[8-(3-methyl-1-benzofuran-5-yl)quinoxalin-6-yl]amino}pyridin-1-ium-1-olate (48 mg; 0.10 mmol; 50%; yellow powder; HPLC purity: 95.8%).

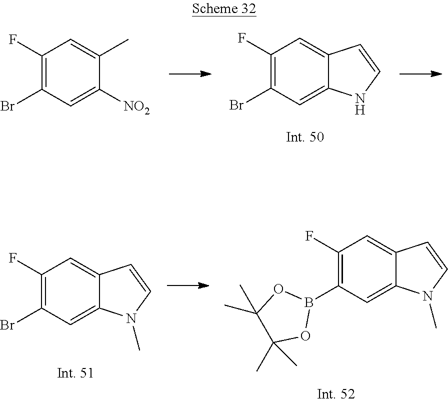 [SMILES: Cc1cc(F)c(Br)cc1[N+](=O)[O-] ; Fc1cc2cc[nH]c2cc1Br ; Cn1ccc2cc(F)c(Br)cc21 ; Cn1ccc2cc(F)c(B3OC(C)(C)C(C)(C)O3)cc21]

### Intermediate 50

### 6-Bromo-5-fluoro-1H-indole

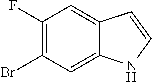 [SMILES: Fc1cc2cc[nH]c2cc1Br]

The title compound is prepared according to a procedure identical to the one described for intermediate 47, using 1-bromo-2-fluoro-4-methyl-5-nitrobenzene (1.3 g; 5.56 mmol; 1 eq.), N,N-dimethylformamide diisopropyl acetal (2.6 mL; 12.22 mmol; 2.2 eq.), DMF (5 mL), triethylamine (0.85 mL; 6.11 mmol; 1.1 eq.), acetic acid (40 mL), iron (6.2 g; 111.10 mmol; 20 eq.) and silica (6 g). Purification by FCC (20% DCM in hexane) affords 6-bromo-5-fluoro-1H-indole (693 mg; 3.24 mmol; 58%; white solid; UPLC purity: 100%).

### Intermediate 51

### 6-Bromo-5-fluoro-1-methyl-1H-indole

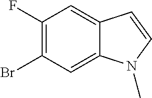

The title compound is prepared according to General Procedure 14 described for Intermediate 45, using 6-bromo-5-fluoro-1H-indole (Intermediate 50, 220 mg; 1.03 mmol; 1 eq.), sodium hydride (60% in mineral oil, 0.08 g; 2.06 mmol; 2 eq.) and iodomethane (0.08 mL; 1.34 mmol; 1.3 eq.). Purification by FCC (10% DCM in hexane) affords 6-bromo-5-fluoro-1-methyl-1H-indole (199 mg; 0.83 mmol; 81%; white solid; UPLC purity: 95%).

### Intermediate 52

### 5-Fluoro-1-methyl-6-(4,4,5,5-tetramethyl-[1,3,2]dioxaborolan-2-yl)-1H-indole

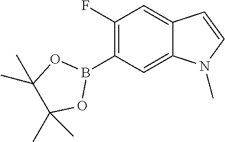 [SMILES: Cn1ccc2cc(F)c(B3OC(C)(C)C(C)(C)O3)cc21]

The title compound is prepared according to General Procedure 8 described for Intermediate 14, using 6-bromo-5-fluoro-1-methyl-1H-indole (Intermediate 51, 190 mg; 0.83 mmol; 1 eq.), bis(pinacolato)diboron (0.28 g; 1.08 mmol; 1.3 eq.), potassium acetate (0.16 g; 1.67 mmol; 2 eq.) and Pd(dppf)Cl₂ (6 mg; 0.01 mmol; 0.01 eq.) in dioxane (5 mL). Crude 5-fluoro-1-methyl-6-(4,4,5,5-tetramethyl-[1,3,2]dioxaborolan-2-yl)-1H-indole (146 mg; 0.48 mmol; 57%; UPLC purity: 90%) is used in the next step without further purification.

### Example 73

### 8-(5-Fluoro-1-methyl-1H-indol-6-yl)-N-(4-methanesulfonylpyridin-3-yl)quinoxalin-6-amine

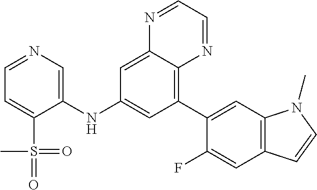 [SMILES: Cn1ccc2cc(F)c(-c3cc(Nc4cnccc4S(C)(=O)=O)cc4nccnc34)cc21]

The title compound is prepared according to General Procedure 17 described in Example 66, using sodium carbonate (154 mg; 1.45 mmol; 5 eq.), 5-fluoro-1-methyl-6-(4,4,5,5-tetramethyl-[1,3,2]dioxaborolan-2-yl)-1H-indole (Intermediate 52, 88 mg; 0.32 mmol; 1.1 eq.), 8-chloro-N-(4-methanesulfonylpyridin-3-yl)quinoxalin-6-amine (Intermediate 3B, 100 mg; 0.29 mmol; 1 eq.) in ethanol (1 mL), water (1 mL) and toluene (2 mL). Conditions: 100° C., overnight. Purification by FCC (4% iPrOH in chloroform) affords 8-(5-fluoro-1-methyl-1H-indol-6-yl)-N-(4-methanesulfonylpyridin-3-yl)quinoxalin-6-amine (25 mg; 0.05 mmol; 18%; yellow powder; HPLC purity: 95.8%).

### Example 74

### N-(4-Methanesulfonylpyridin-3-yl)-8-(2-methoxy-5-methylphenyl)quinoxalin-6-amine

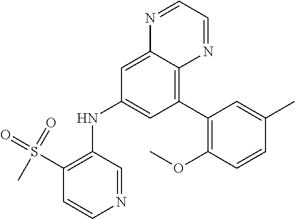 [SMILES: COc1ccc(C)cc1-c1cc(Nc2cnccc2S(C)(=O)=O)cc2nccnc12]

The title compound is prepared according to General Procedure 17 described in Example 66, using sodium carbonate (115 mg; 1.09 mmol; 5 eq.), (2-methoxy-5-methyl-phenyl)boronic acid (40 mg; 0.24 mmol; 1.1 eq.), 8-chloro-N-(4-methanesulfonylpyridin-3-yl)quinoxalin-6-amine (Intermediate 3B, 75 mg; 0.22 mmol; 1 eq.) and tetrakis(triphenylphosphine)palladium(0) in ethanol (1 mL), water (1 mL) and toluene (2 mL). Conditions: 100° C., overnight. Purification by FCC (4% iPrOH in chloroform) affords N-(4-methanesulfonylpyridin-3-yl)-8-(2-methoxy-5-methylphenyl)quinoxalin-6-amine (18 mg; 0.04 mmol; 19%; yellow powder; HPLC purity: 98.5%).

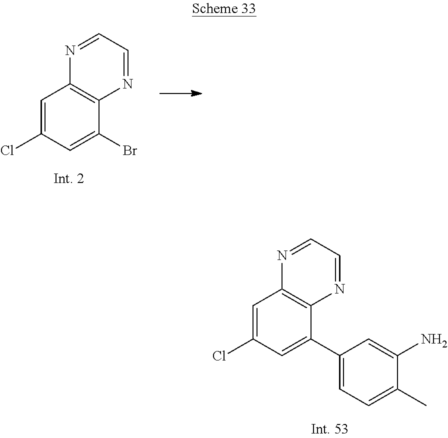 [SMILES: Clc1cc(Br)c2nccnc2c1 ; Cc1ccc(-c2cc(Cl)cc3nccnc23)cc1N]

### Intermediate 53

### 5-(7-Chloroquinoxalin-5-yl)-2-methylaniline

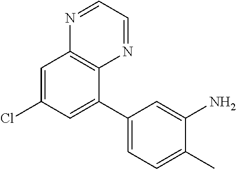 [SMILES: Cc1ccc(-c2cc(Cl)cc3nccnc23)cc1N]

A pressure vessel is charged with 5-bromo-7-chloroquinoxaline (Intermediate 2, 400 mg; 1.64 mmol; 1 eq.), 3-amino-4-methylphenylboronic acid (273 mg; 1.81 mmol; 1.10 eq.), DIPEA (0.57 mL; 3.29 mmol; 2 eq.), dioxane (3 mL) and water (3 mL). The suspension is sparged with argon and Pd(dppf)Cl₂ (120 mg; 0.16 mmol; 0.10 eq.) is added. The reaction mixture is sealed and heated at 85° C. for 3 hours. After coming back to room temperature, the mixture is filtered through a pad of celite, the filtrate is diluted with DCM and washed with water. The organic phase is washed with brine, dried over Na₂SO₄ and the solvent is evaporated. The crude product is purified by FCC (EtOAc gradient in hexane) to afford 5-(7-chloroquinoxalin-5-yl)-2-methylaniline (349 mg; 1.25 mmol; 76%; yellow solid; UPLC purity: 96%).

### Example 75

### 8-(3-Amino-4-methylphenyl)-N-(4-methanesulfonylpyridin-3-yl)quinoxalin-6-amine

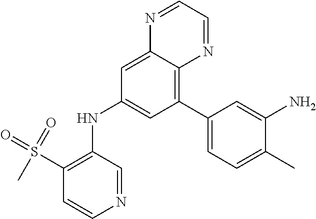 [SMILES: Cc1ccc(-c2cc(Nc3cnccc3S(C)(=O)=O)cc3nccnc23)cc1N]

The title compound is prepared according to General Procedure 1 described in Example 1, using 5-(7-chloroquinoxalin-5-yl)-2-methylaniline (Intermediate 53, 140 mg; 0.52 mmol; 1 eq.), 4-methanesulfonylpyridin-3-ylamine hydrochloride (162 mg; 0.78 mmol; 1.5 eq.), cesium carbonate (507 mg; 1.56 mmol; 3 eq.), BINAP (65 mg; 0.10 mmol; 0.2 eq.) and Pd(OAc)₂ (12 mg; 0.05 mmol; 0.1 eq.) in dioxane (3 mL). Conditions: 130° C., 2 hours. Purification by FCC (EtOAc gradient in hexane followed by MeOH gradient in EtOAc) affords 8-(3-Amino-4-methylphenyl)-N-(4-methanesulfonylpyridin-3-yl)quinoxalin-6-amine (85 mg; 0.20 mmol; 38%; yellow-brown solid; HPLC purity: 96.9%).

### Example 76

### 8-[2-(Dimethylamino)-5-methylphenyl]-N-(4-methanesulfonylpyridin-3-yl)quinoxalin-6-amine

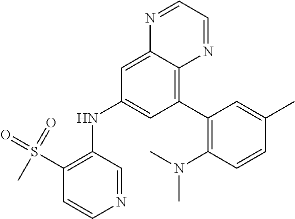 [SMILES: Cc1ccc(N(C)C)c(-c2cc(Nc3cnccc3S(C)(=O)=O)cc3nccnc23)c1]

The title compound is prepared according to General Procedure 17 described in Example 66, using sodium carbonate (115 mg; 1.09 mmol; 5 eq.), [2-(dimethylamino)-5-methyl-phenyl]boronic acid (43 mg; 0.24 mmol; 1.10 eq.), 8-chloro-N-(4-methanesulfonylpyridin-3-yl)quinoxalin-6-amine (Intermediate 3B, 75 mg; 0.22 mmol; 1 eq.) and tetrakis(triphenylphosphine)palladium(0) (13 mg; 0.01 mmol; 0.05 eq.), in ethanol (0.5 mL), water (0.5 mL) and toluene (1 mL). Conditions: 100° C., overnight. The crude compound is purified by recrystallization from MeOH overnight at room temperature to give 8-[2-(dimethylamino)-5-methylphenyl]-N-(4-methanesulfonylpyridin-3-yl)quinoxalin-6-amine (63 mg; 0.15 mmol; 67%; yellow crystals; HPLC purity: 99.9%).

### Example 81

### N-(3-Methanesulfonylpyridin-2-yl)-8-(1-methyl-1H-indol-6-yl)quinoxalin-6-amine

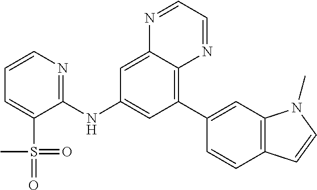 [SMILES: Cn1ccc2ccc(-c3cc(Nc4ncccc4S(C)(=O)=O)cc4nccnc34)cc21]

The title compound is prepared according to General Procedure 1, described in Example 1, using 8-(1-methyl-1H-indol-6-yl)quinoxalin-6-amine (Intermediate 4, 100 mg; 0.34 mmol; 1 eq.), 2-chloro-3-methanesulfonyl-pyridine (82 mg; 0.41 mmol; 1.20 eq.), cesium carbonate (279 mg; 0.85 mmol; 2.50 eq.) BINAP (22 mg; 0.03 mmol; 0.10 eq.) and palladium(II) acetate (8 mg; 0.03 mmol; 0.10 eq.) in anhydrous dioxane (2 mL). Conditions: 120° C., overnight. Purification by FCC (0% to 100% EtOAc gradient in hexane) gives N-(3-methanesulfonylpyridin-2-yl)-8-(1-methyl-1H-indol-6-yl)quinoxalin-6-amine (95 mg, 0.21 mmol, 61.7%; yellow powder; HPLC purity: 94.6%).

### Example 82

### 1-[4-(3-{[8-(1-Methyl-1H-indol-6-yl)quinoxalin-6-yl]amino}pyridin-4-yl)piperazin-1-yl]ethan-1-one

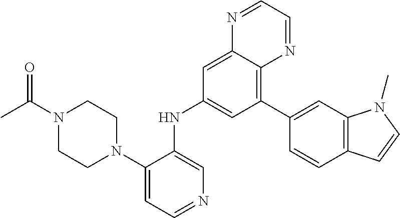 [SMILES: CC(=O)N1CCN(c2ccncc2Nc2cc(-c3ccc4ccn(C)c4c3)c3nccnc3c2)CC1]

The title compound is prepared according to General Procedure 1 described in Example 1, using 7-chloro-5-(1-methyl-1H-indol-6-yl)quinoxaline (Intermediate 2B, 60 mg; 0.20 mmol; 1 eq.), I-[4-(3-aminopyridin-4-yl)piperazin-1-yl]ethan-1-one (67 mg; 0.30 mmol; 1.50 eq.), cesium carbonate (198 mg; 0.60 mmol; 3 eq.), BINAP (13 mg; 0.02 mmol; 0.10 eq.) and palladium(II) acetate (5 mg; 0.02 mmol; 0.10 eq.). Conditions: 150° C., 2 hours. Purification by reversed-phase preparative HPLC (column: Gemini NX C18 5u 110A (100×30 mm), ACN gradient in water) affords 1-[4-(3-{[8-(1-methyl-1H-indol-6-yl)quinoxalin-6-yl]amino}pyridin-4-yl)piperazin-1-yl]ethan-1-one (40 mg; 0.08 mmol; 39%; yellow powder; HPLC purity: 93.1%).

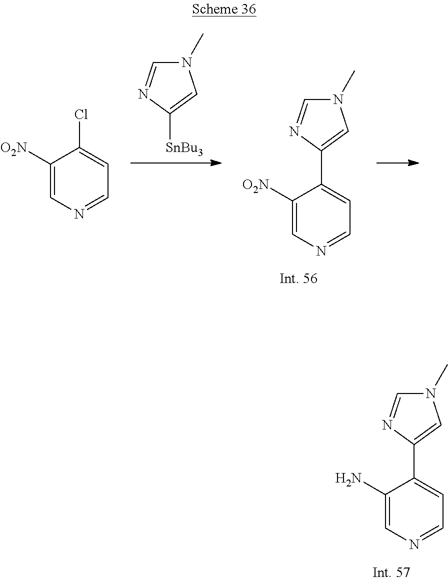

### Intermediate 56

### 4-(1-methyl-1H-imidazol-4-yl)-3-nitropyridine

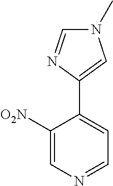

In a microwave vial under argon, tetrakis(triphenylphosphine)palladium(0) (18 mg; 0.02 mmol; 0.05 eq.) is added to a deaerated mixture of 4-chloro-3-nitropyridine (50 mg; 0.32 mmol; 1 eq.), 1-methyl-4-tributylstannanyl-1H-imidazole (176 mg; 0.47 mmol; 1.50 eq.) in anhydrous DMF (2 mL). The reaction mixture is flushed with argon, the vial is sealed an heated under microwave irradiation at 140° C. for 1 h. The residue obtained after solvent evaporation is purified by FCC (0% to 5% MeOH gradient in DCM) to afford 4-(1-methyl-1H-imidazol-4-yl)-3-nitropyridine (65 mg; 0.30 mmol; 94%; yellow-brown powder; UPLC purity: 93%).

### Intermediate 57

### 4-(1-Methyl-1H-imidazol-4-yl)pyridin-3-amine

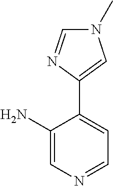

The title compound is prepared according to General Procedure 4 described for Intermediate 6, using 4-(1-methyl-1H-imidazol-4-yl)-3-nitropyridine (Intermediate 56, 40 mg, 0.18 mmol, 1 eq.), 10% Pd/C (10 mg) in EtOAc (3 mL). The crude 4-(1-methyl-1H-imidazol-4-yl)pyridin-3-amine obtained (28 mg, 89%, white solid; UPLC purity: 100%) is used in the next step without further purification.

### Example 83

### N-[4-(1-Methyl-1H-imidazol-4-yl)pyridin-3-yl]-8-(1-methyl-1H-indol-6-yl)quinoxalin-6-amine

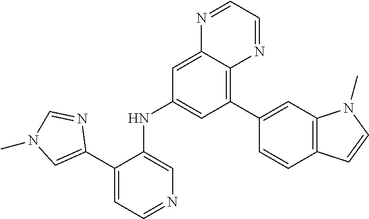 [SMILES: Cn1cnc(-c2ccncc2Nc2cc(-c3ccc4ccn(C)c4c3)c3nccnc3c2)c1]

The title compound is prepared according to General Procedure 1 described in Example 1, using 7-chloro-5-(1-methyl-1H-indol-6-yl)quinoxaline (Intermediate 2B, 32 mg; 0.11 mmol; 1 eq.), 4-(1-methyl-1H-imidazol-4-yl)pyridin-3-amine (Intermediate 57, 37 mg; 0.16 mmol; 1.50 eq.), cesium carbonate (105 mg; 0.32 mmol; 3 eq.), BINAP (27 mg; 0.04 mmol; 0.40 eq.) and palladium(II) acetate (10 mg; 0.04 mmol; 0.40 eq.) in dioxane (2 mL). Conditions: 150° C., 1 hour. Purification by FCC (0% to 5% MeOH gradient in DCM) affords N-[4-(1-methyl-1H-imidazol-4-yl)pyridin-3-yl]-8-(1-methyl-1H-indol-6-yl)quinoxalin-6-amine (15 mg; 0.03 mmol; 31%; yellow powder; HPLC purity: 95.7%).

### Example 84

### 8-(1-Methyl-1H-indol-6-yl)-N-{2H,3H,4H-pyrido[4,3-b][1,4]oxazin-8-yl}quinoxalin-6-amine

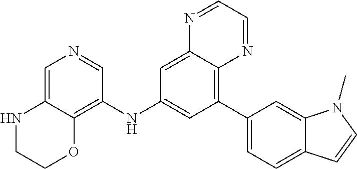 [SMILES: Cn1ccc2ccc(-c3cc(Nc4cncc5c4OCCN5)cc4nccnc34)cc21]

A pressure vessel is charged with 8-bromo-3,4-dihydro-2H-pyrido[4,3-b][1,4]oxazine (50 mg; 0.23 mmol; 1 eq.), tBuONa (67 mg; 0.70 mmol; 3 eq.), 8-(1-methyl-1H-indol-6-yl)quinoxalin-6-amine (Intermediate 4, 96 mg; 0.35 mmol; 1.50 eq.), Brettphos (12 mg; 0.02 mmol; 0.10 eq.) and anhydrous dioxane (2 mL). The reaction mixture is sparged with argon for 15 minutes and BrettPhos precatalyst (10 mg; 0.01 mmol; 0.05 eq.) is added. The vessel is sealed and the reaction mixture is stirred at 110° C. for 18 h. The reaction mixture is then diluted with EtOAc/MeOH and filtered through a pad of celite. The filtrate is evaporated and the residue is dissolved in EtOAc. The resulting solution is washed with brine, dried over MgSO₄ and evaporated. The crude product is purified by FCC (0% to 60% of EtOAc gradient in hexane) to 8-(1-methyl-1H-indol-6-yl)-N-{2H,3H,4H-pyrido[4,3-b][1,4]oxazin-8-yl}quinoxalin-6-amine (42 mg; 0.10 mmol; 42%; brown orange powder; HPLC purity: 96%).

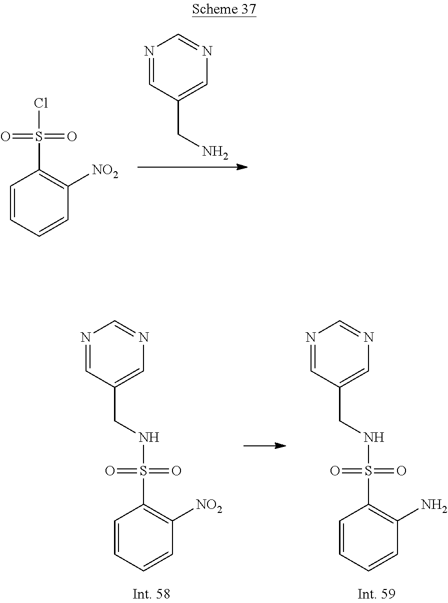 [SMILES: O=[N+]([O-])c1ccccc1S(=O)(=O)Cl ; NCc1cncnc1 ; O=[N+]([O-])c1ccccc1S(=O)(=O)NCc1cncnc1 ; Nc1ccccc1S(=O)(=O)NCc1cncnc1]

### Intermediate 58

### 2-Nitro-N-(pyrimidin-5-ylmethyl)benzene-1-sulfonamide

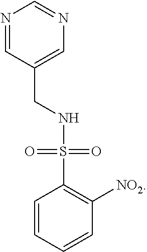 [SMILES: O=[N+]([O-])c1ccccc1S(=O)(=O)NCc1cncnc1]

Pyrimidin-5-ylmethanamine (100 mg; 0.92 mmol; 1 eq.), 2-nitro-benzene-sulfonyl chloride (203 mg; 0.92 mmol; 1 eq.) and triethylamine (0.13 mL; 0.92 mmol; 1 eq.) are stirred for 1.5 h at room temperature in DCM (5 mL). The reaction mixture then is diluted with EtOAc and washed with water and brine. The organic layer is dried over Na₂SO₄ and evaporated to obtain crude 2-nitro-N-(pyrimidin-5-ylmethyl)benzene-1-sulfonamide (0.25 g; 0.78 mmol; 84.7%; UPLC purity: 91%) which is used in the next step without further purification.

### Intermediate 59

### 2-Amino-N-(pyrimidin-5-ylmethyl)benzene-1-sulfonamide

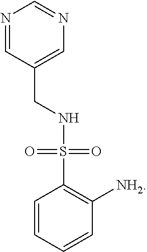 [SMILES: Nc1ccccc1S(=O)(=O)NCc1cncnc1]

Hydrazine monohydrate (0.05 mL; 0.68 mmol; 5 eq.) is added dropwise to a suspension of Raney Nickel (around 50 mg) and 2-nitro-N-(pyrimidin-5-ylmethyl)benzene-1-sulfonamide (Intermediate 58, 50 mg; 0.14 mmol; 1 eq.) in iPrOH (3 mL). The reaction mixture is stirred at room temperature for 1 h, filtered through celite and evaporated to afford crude 2-amino-N-(pyrimidin-5-ylmethyl)benzene-1-sulfonamide (38 mg; 0.14 mmol; >100%; white solid; UPLC purity: 99.8%).

### Example 85

### 2-{[8-(1-Methyl-1H-indol-6-yl)quinoxalin-6-yl]amino}-N-[(pyrimidin-5-yl)methyl]benzene-1-sulfonamide

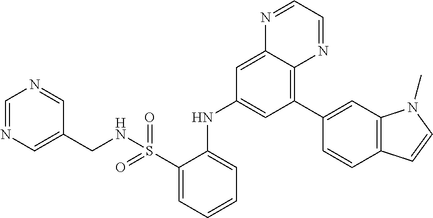 [SMILES: Cn1ccc2ccc(-c3cc(Nc4ccccc4S(=O)(=O)NCc4cncnc4)cc4nccnc34)cc21]

The title compound is prepared according to General Procedure 7 described in Example 14, using 7-chloro-5-(1-methyl-1H-indol-6-yl)quinoxaline (Intermediate 2B, 33 mg; 0.11 mmol; 0.75 eq.), BrettPhos (8 mg; 0.01 mmol; 0.10 eq.) and BrettPhos precatalyst (11 mg; 0.01 mmol; 0.10 eq.), 2-amino-N-(pyrimidin-5-ylmethyl)benzene-1-sulfonamide (Intermediate 59, 38 mg; 0.14 mmol; 1 eq.) and LiHMDS (1M THF solution, 430 μl; 0.43 mmol; 3 eq.). Conditions: 60° C., overnight. Purification by FCC (0% to 100% EtOAc gradient in hexane, followed by 0% to 10% MeOH gradient in EtOAc) and trituration in DCM/hexane, affords 2-{[8-(1-methyl-1H-indol-6-yl)quinoxalin-6-yl]amino}-N-[(pyrimidin-5-yl)methyl]benzene-1-sulfonamide (24 mg; 0.04 mmol; 31%; yellow powder; HPLC purity: 96.1%).

### Example 88

### 2-{[8-(1-Methyl-1H-indol-6-yl)quinoxalin-6-yl]amino}benzonitrile

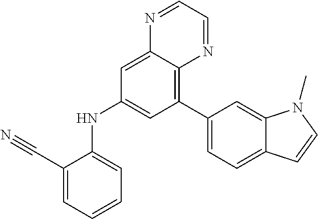 [SMILES: Cn1ccc2ccc(-c3cc(Nc4ccccc4C#N)cc4nccnc34)cc21]

The title compound is prepared according to General Procedure 1 described in Example 1, using 7-chloro-5-(1-methyl-1H-indol-6-yl)quinoxaline (Intermediate 2B, 250 mg; 0.82 mmol; 1 eq.), 2-aminobenzonitrile (122 mg; 0.98 mmol; 1.20 eq.), cesium carbonate (808 mg; 2.46 mmol; 3 eq.), BINAP (52 mg; 0.08 mmol; 0.10 eq.), palladium(II) acetate (19 mg; 0.08 mmol; 0.10 eq.) and dioxane (10 mL). Conditions: 150° C., 1.5 hours. Purification by FCC (0% to 40% EtOAc gradient in hexane) affords 2-{[8-(1-methyl-1H-indol-6-yl)quinoxalin-6-yl]amino}benzonitrile (270 mg; 0.71 mmol; 86%; yellow powder; HPLC purity: 95.4%).

### Example 89, General Procedure 20

### 2-{[8-(1-methyl-1H-indol-6-yl)quinoxalin-6-yl]amino}benzamide

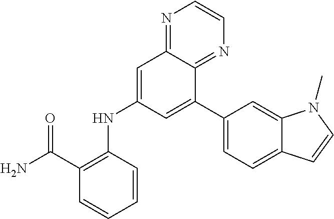 [SMILES: Cn1ccc2ccc(-c3cc(Nc4ccccc4C(N)=O)cc4nccnc34)cc21]

A round-bottom flask is charged with tBuOH (10 mL), KOH (66 mg; 1.18 mmol; 9 eq.) and 2-{[8-(1-methyl-1H-indol-6-yl)quinoxalin-6-yl]amino}-benzonitrile (50 mg; 0.13 mmol; 1 eq.), and the mixture is refluxed 85° C. for 40 h under argon. After coming back to room temperature, the reaction mixture is diluted with EtOAc and water, and neutralized with 1 M HCl. The aqueous layer is extracted with EtOAc and the combined organic layers are washed with water, dried over Na₂SO₄ and filtered through a pad of celite. The filtrate is concentrated in vacuo and the crude product is purified by FCC (30% to 70% EtOAc gradient in hexane) to afford 2-{[8-(1-methyl-1H-indol-6-yl)quinoxalin-6-yl]amino}benzamide (18 mg; 0.04 mmol; 33%; yellow orange powder; HPLC purity: 95.3%).

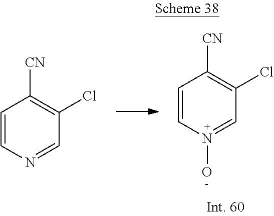

### Intermediate 60

### 3-Chloro-4-cyanopyridin-1-ium-1-olate

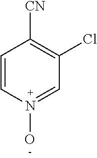

A mixture of 3-chloro-isonicotinonitrile (110 mg; 0.75 mmol; 1 eq.) and mCPBA (338 mg; 1.51 mmol; 2 eq.) in anhydrous DCM (5 mL) is stirred at room temperature overnight. A new portion of mCPBA (39 mg; 0.23 mmol; 0.30 eq.) is added and the reaction mixture is further stirred for 16 h at room temperature. It is then diluted with DCM, washed with saturated aqueous NaHCO₃, 1 M aqueous NaOH and brine, dried over Na₂SO₄ and concentrated in vacuo to afford crude 3-chloro-4-cyanopyridin-1-ium-1-olate (116 mg; 0.72 mmol; 95%; white powder; UPLC purity: 96%).

### Example 90

### 4-Cyano-3-{[8-(1-methyl-1H-indol-6-yl)quinoxalin-6-yl]amino}pyridin-1-ium-1-olate

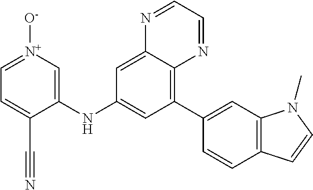 [SMILES: Cn1ccc2ccc(-c3cc(Nc4c[n+]([O-])ccc4C#N)cc4nccnc34)cc21]

The title compound is prepared according to General Procedure 1 described in Example 1, using 8-(1-methyl-1H-indol-6-yl)quinoxalin-6-amine (Intermediate 4, 243 mg; 0.86 mmol; 1.20 eq.), 3-chloro-4-cyanopyridin-1-ium-1-olate (Intermediate 60, 116 mg; 0.72 mmol; 1 eq.), cesium carbonate (711 mg; 2.16 mmol; 3 eq.), BINAP (46 mg; 0.07 mmol; 0.10 eq.), palladium(II) acetate (17 mg; 0.07 mmol; 0.10 eq.) in dioxane (6 mL). Conditions: 150° C., 1.5 hours. Purification by FCC (0% to 5% MeOH gradient in DCM) affords 4-cyano-3-{[8-(1-methyl-1H-indol-6-yl)quinoxalin-6-yl]amino}pyridin-1-ium-1-olate (185 mg; 0.47 mmol; 65%; yellow powder; HPLC purity: 99.1%).

### Example 91

### 3-{Methyl[8-(1-methyl-1H-indol-6-yl)quinoxalin-6-yl]amino}pyridine-4-carbonitrile

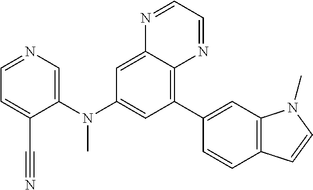 [SMILES: CN(c1cc(-c2ccc3ccn(C)c3c2)c2nccnc2c1)c1cnccc1C#N]

A solution of 2-{[8-(1-Methyl-1H-indol-6-yl)quinoxalin-6-yl]amino}benzonitrile (Example 88, 20 mg; 0.05 mmol; 1 eq.) in anhydrous THF (3 mL) is cooled in an ice bath. NaH (60% in mineral oil, 4 mg; 0.10 mmol; 2 eq.) is added and the mixture is stirred for 10 min. Iodomethane (4 μl; 0.06 mmol; 1.20 eq.) is added and the reaction mixture is stirred for another 2 h at room temperature. It is then poured onto ice and extracted twice with EtOAc. The combined organic layers are washed with brine, dried over Na₂SO₄, filtered through a pad of celite and concentrated in vacuo. The crude product is purified by FCC (50% to 80% EtOAc gradient in hexane) to afford 3-{methyl[8-(1-methyl-1H-indol-6-yl)quinoxalin-6-yl]amino}pyridine-4-carbonitrile (13 mg; 0.03 mmol; 63%; yellow powder; HPLC purity: 98.7%).

### Example 92

### 3-{[8-(1-Methyl-1H-indol-6-yl)quinoxalin-6-yl]amino}-N-(1-methyl-1H-pyrazol-4-yl)pyridine-4-carboxamide

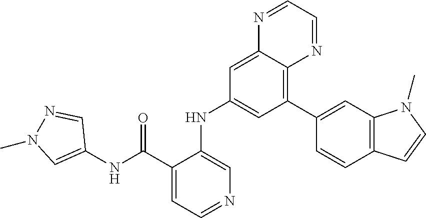 [SMILES: Cn1cc(NC(=O)c2ccncc2Nc2cc(-c3ccc4ccn(C)c4c3)c3nccnc3c2)cn1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 50 mg; 0.13 mmol; 1 eq.), 1-methyl-1H-pyrazol-4-ylamine (19 mg; 0.19 mmol; 1.50 eq.), EDC.HCl (59 mg; 0.3 mmol; 2.40 eq.), HOBt hydrate (47 mg; 0.3 mmol; 2.40 eq.), triethylamine (0.08 mL; 0.63 mmol; 5 eq.) and dioxane (7 mL). Purification by FCC (0% to 10% MeOH gradient in DCM) affords 3-{[8-(1-methyl-1H-indol-6-yl)quinoxalin-6-yl]amino}-N-(1-methyl-1H-pyrazol-4-yl)pyridine-4-carboxamide (37 mg; 0.08 mmol; 61%; yellow powder; HPLC purity: 99.1%).

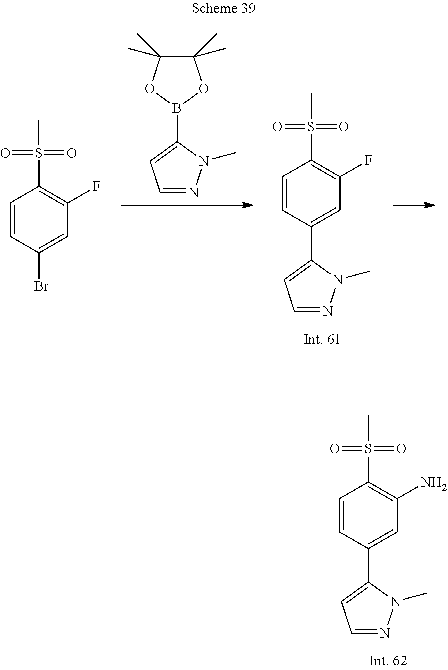

### Intermediate 61

### 5-(3-Fluoro-4-methanesulfonylphenyl)-1-methyl-1H-pyrazole

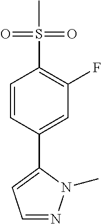 [SMILES: Cn1nccc1-c1ccc(S(C)(=O)=O)c(F)c1]

The title compound is prepared according to General Procedure 16 described in Example 62, using 4-bromo-2-fluoro-1-methanesulfonylbenzene (100 mg; 0.40 mmol; 1 eq.), 1-methyl-5-(4,4,5,5-tetramethyl-[1,3,2]dioxaborolan-2-yl)-1H-pyrazole (164 mg; 0.79 mmol; 2 eq.), cesium carbonate (407 mg; 1.19 mmol; 3 eq.), Pd(dppf)₂CH₂Cl₂ (17 mg; 0.02 mmol; 0.05 eq.), dioxane (2 mL) and water (1 mL). Conditions: 100° C., overnight. Purification by FCC (0% to 50% EtOAc gradient in hexane) affords 5-(3-fluoro-4-methanesulfonyl-phenyl)-1-methyl-1H-pyrazole (95 mg; 0.37 mmol; 94%; UPLC purity: 99.5%).

### Intermediate 62, General Procedure 19

### 2-Methanesulfonyl-5-(1-methyl-1H-pyrazol-5-yl)aniline

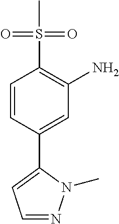 [SMILES: Cn1nccc1-c1ccc(S(C)(=O)=O)c(N)c1]

A pressure vessel is charged with 5-(3-fluoro-4-methanesulfonylphenyl)-1-methyl-1H-pyrazole (Intermediate 61, 95 mg; 0.37 mmol; 1 eq.), DMSO (1 mL) and 25% aqueous ammonia (1.2 mL; 7.43 mmol; 20 eq.). The vessel is sealed and the reaction mixture is stirred at 140° C. overnight. After coming back to room temperature, ethyl acetate and water are added and the organic phase is washed twice with brine, dried (Na₂SO₄) and concentrated in vacuo. The crude product is purified by FCC (0% to 50% MeOH gradient in EtOAc) to afford 2-methanesulfonyl-5-(1-methyl-1H-pyrazol-5-yl)aniline (96 mg; 0.37 mmol; 99%; white powder; UPLC purity: 97%).

### Example 93

### N-[2-Methanesulfonyl-5-(1-methyl-1H-pyrazol-5-yl)phenyl]-8-(1-methyl-1H-indol-6-yl)quinoxalin-6-amine

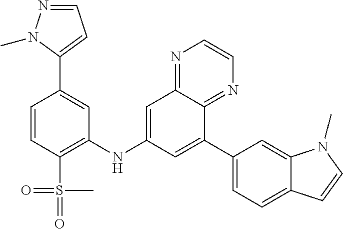 [SMILES: Cn1nccc1-c1ccc(S(C)(=O)=O)c(Nc2cc(-c3ccc4ccn(C)c4c3)c3nccnc3c2)c1]

The title compound is prepared according to General Procedure 1 described in Example 1, using 7-chloro-5-(1-methyl-1H-indol-6-yl)quinoxaline (Intermediate 2B, 60 mg; 0.19 mmol; 1 eq.), 2-methanesulfonyl-5-(1-methyl-1H-pyrazol-5-yl)aniline (Intermediate 62, 76 mg; 0.29 mmol; 1.50 eq.), cesium carbonate (319 mg; 0.97 mmol; 5 eq.), BINAP (12 mg; 0.02 mmol; 0.10 eq.), palladium(II) acetate (5 mg; 0.02 mmol; 0.10 eq.) and dioxane (2 mL). Conditions: 150° C., 1 hour. Purification by FCC (0% to 50% EtOAc gradient in hexane) affords N-[2-methanesulfonyl-5-(1-methyl-1H-pyrazol-5-yl)phenyl]-8-(1-methyl-1H-indol-6-yl)quinoxalin-6-amine (87 mg; 0.16 mmol; 84%; pale yellow powder; HPLC purity: 94.8%).

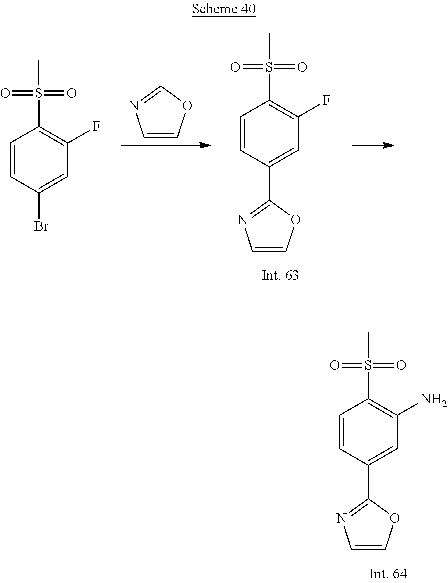

### Intermediate 63

### 2-(3-Fluoro-4-methanesulfonylphenyl)-1,3-oxazole

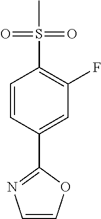 [SMILES: CS(=O)(=O)c1ccc(-c2ncco2)cc1F]

A microwave vial is charged with 4-bromo-2-fluoro-1-methanesulfonyl-benzene (200 mg; 0.79 mmol; 1 eq.), potassium carbonate (328 mg; 2.37 mmol; 3 eq.), pivalic acid (3 mg; 0.03 mmol; 0.04 eq.), palladium(II) acetate (12 mg; 0.05 mmol; 0.06 eq.), RuPhos (47 mg; 0.10 mmol; 0.13 eq.), oxazole (0.10 mL; 1.58 mmol; 2 eq.) and anhydrous toluene (4 mL). The mixture is sparged with argon, the vial is sealed and the reaction mixture is stirred at 110° C. overnight. After coming back to room temperature, the reaction mixture is evaporated to a volume of around 2 mL, and the crude product is purified by FCC (0% to 50% EtOAc gradient in hexane) to give 2-(3-fluoro-4-methanesulfonylphenyl)-1,3-oxazole (83 mg; 0.33 mmol; 42%; white powder; UPLC purity: 97%).

### Intermediate 64

### 2-Methanesulfonyl-5-(1,3-oxazol-2-yl)aniline

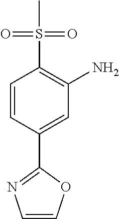 [SMILES: CS(=O)(=O)c1ccc(-c2ncco2)cc1N]

The title compound is prepared according to General Procedure 19 described for Intermediate 62, using 2-methanesulfonyl-5-(1,3-oxazol-2-yl)aniline (Intermediate 64, 80 mg; 0.32 mmol; 1 eq.), 28% aqueous ammonia (0.87 mL; 6.30 mmol; 20 eq.) and DMSO (2 mL). Conditions: 120° C., 12 hours. The crude 2-methanesulfonyl-5-(1,3-oxazol-2-yl)aniline (73 mg; 0.29 mmol; 93%; yellow oil; UPLC purity: 96%) is used in the next step without further purification.

### Example 94

### N-[2-Methanesulfonyl-5-(1,3-oxazol-2-yl)phenyl]-8-(1-methyl-1H-indol-6-yl)quinoxalin-6-amine

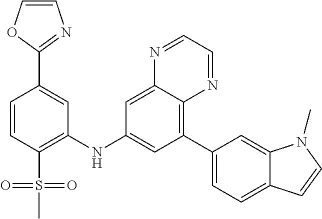 [SMILES: Cn1ccc2ccc(-c3cc(Nc4cc(-c5ncco5)ccc4S(C)(=O)=O)cc4nccnc34)cc21]

The title compound is prepared according to General Procedure 1 described in Example 1, using 7-chloro-5-(1-methyl-1H-indol-6-yl)quinoxaline (Intermediate 2B, 30 mg; 0.10 mmol; 1 eq.), 2-methanesulfonyl-5-(1,3-oxazol-2-yl)aniline (Intermediate 64, 36 mg; 0.15 mmol; 1.50 eq.), BINAP (6 mg; 0.01 mmol; 0.10 eq.), palladium(II) acetate (2 mg; 0.01 mmol; 0.10 eq.), cesium carbonate (160 mg; 0.49 mmol; 5 eq.) and dioxane (2 mL). Conditions: 150° C., 2 hours. Purification FCC (0% to 50% EtOAc gradient in hexane) affords N-[2-methanesulfonyl-5-(1,3-oxazol-2-yl)phenyl]-8-(1-methyl-1H-indol-6-yl)quinoxalin-6-amine (37 mg; 0.07 mmol; 73%; pale yellow powder; HPLC purity: 94.8%).

### Example 95

### 3-{Methyl[8-(1-methyl-1H-indol-6-yl)quinoxalin-6-yl]amino}pyridine-4-carboxamide

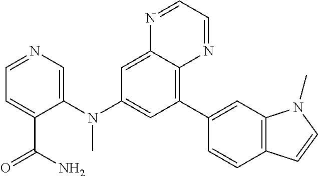 [SMILES: CN(c1cc(-c2ccc3ccn(C)c3c2)c2nccnc2c1)c1cnccc1C(N)=O]

The title compound is prepared according to General Procedure 20 described in Example 89, using 3-{methyl[8-(1-methyl-1H-indol-6-yl)quinoxalin-6-yl]amino}pyridine-4-carbonitrile (Example 91, 46 mg; 0.11 mmol; 1 eq.), KOH (19 mg; 0.34 mmol; 3 eq.) and tBuOH (3 mL). Conditions: 130° C., 2 hours. Purification by FCC (30% to 100% EtOAc gradient in hexane followed by 0% to 5% MeOH gradient in EtOAc) affords 3-{methyl[8-(1-methyl-1H-indol-6-yl)quinoxalin-6-yl]amino}pyridine-4-carboxamide (26 mg; 0.06 mmol; 56%; yellow solid; HPLC purity: 97.8%).

### Example 96

### 3-{[8-(1-Methyl-1H-indol-6-yl)quinoxalin-6-yl]amino}-N-phenylpyridine-4-carboxamide

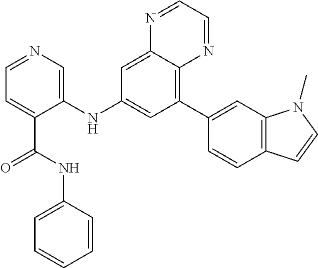 [SMILES: Cn1ccc2ccc(-c3cc(Nc4cnccc4C(=O)Nc4ccccc4)cc4nccnc34)cc21]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 50 mg; 0.12 mmol; 1 eq.), aniline (14 μl; 0.15 mmol; 1.25 eq.), EDC.HCl (44 mg; 0.22 mmol; 1.80 eq.), HOBt hydrate (35 mg; 0.22 mmol; 1.80 eq.), triethylamine (0.08 mL; 0.62 mmol; 5 eq.) and dioxane (5 mL). Conditions: room temperature for 24 h. Purification by FCC (0% to 100% EtOAc gradient in hexane followed by 0% to 5% MeOH gradient in EtOAc) affords 3-{[8-(1-methyl-1H-indol-6-yl)quinoxalin-6-yl]amino}-N-phenylpyridine-4-carboxamide (28 mg; 0.06 mmol; 45%; yellow powder; HPLC purity: 92.9%).

### Example 97

### 3-{[8-(1-Methyl-1H-indol-6-yl)quinoxalin-6-yl]amino}-N-(1-methyl-2-oxopiperidin-4-yl)pyridine-4-carboxamide

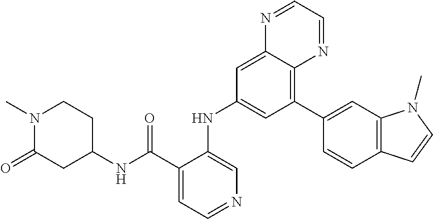 [SMILES: CN1CCC(NC(=O)c2ccncc2Nc2cc(-c3ccc4ccn(C)c4c3)c3nccnc3c2)CC1=O]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 50 mg; 0.12 mmol; 1 eq.), 4-amino-1-methylpiperidin-2-one hydrochloride (27 mg; 0.15 mmol; 1.25 eq.), EDC.HCl (44 mg; 0.22 mmol; 1.80 eq.), HOBt hydrate (35 mg; 0.22 mmol; 1.80 eq.), triethylamine (0.08 mL; 0.62 mmol; 5 eq.) and dioxane (5 mL). Conditions: room temperature for 24 h. Purification by FCC (column: PF—NH2/30 um/6G, 0% to 2% MeOH gradient in DCM) affords 3-{[8-(1-methyl-1H-indol-6-yl)quinoxalin-6-yl]amino}-N-(1-methyl-2-oxopiperidin-4-yl)pyridine-4-carboxamide (53 mg; 0.10 mmol; 82%; yellow powder; HPLC purity: 97.5%).

### Example 98

### N-(1-Acetylazetidin-3-yl)-3-{[8-(1-methyl-1H-indol-6-yl)quinoxalin-6-yl]amino}pyridine-4-carboxamide

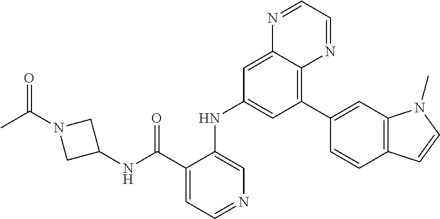 [SMILES: CC(=O)N1CC(NC(=O)c2ccncc2Nc2cc(-c3ccc4ccn(C)c4c3)c3nccnc3c2)C1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 50 mg; 0.12 mmol; 1 eq.), 1-(3-aminoazetidin-1-yl)-ethanone hydrochloride (24 mg; 0.15 mmol; 1.25 eq.), EDC.HCl (44 mg; 0.22 mmol; 1.80 eq.), HOBt hydrate (35 mg; 0.22 mmol; 1.80 eq.), triethylamine (0.08 mL; 0.62 mmol; 5 eq.) and dioxane (5 mL). Conditions: room temperature for 24 h. Purification by FCC (column: PF—NH2/30 um/6G, 0% to 2% MeOH gradient in DCM) affords N-(1-acetylazetidin-3-yl)-3-{[8-(1-methyl-1H-indol-6-yl)quinoxalin-6-yl]amino}pyridine-4-carboxamide (56 mg; 0.11 mmol; 88%; yellow powder; HPLC purity: 95.7%).

### Example 99

### 3-{[8-(1-Methyl-1H-indol-6-yl)quinoxalin-6-yl]amino}-N-(1-methylpyrrolidin-3-yl)pyridine-4-carboxamide

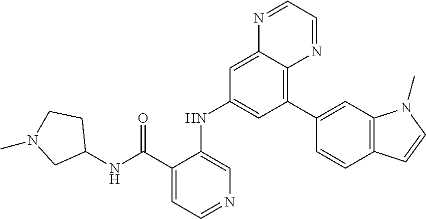 [SMILES: CN1CCC(NC(=O)c2ccncc2Nc2cc(-c3ccc4ccn(C)c4c3)c3nccnc3c2)C1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 50 mg; 0.12 mmol; 1 eq.), 1-methylpyrrolidin-3-ylamine hydrochloride (28 mg; 0.15 mmol; 1.25 eq.), EDC.HCl (44 mg; 0.22 mmol; 1.80 eq.), HOBt hydrate (35 mg; 0.22 mmol; 1.80 eq.), triethylamine (0.08 mL; 0.62 mmol; 5 eq.) and dioxane (5 mL). Conditions: room temperature for 24 h. Purification by FCC (column: PF—NH2/30 um/6G, 0% to 1% MeOH gradient in DCM) affords 3-{[8-(1-methyl-1H-indol-6-yl)quinoxalin-6-yl]amino}-N-(1-methylpyrrolidin-3-yl)pyridine-4-carboxamide (42 mg; 0.08 mmol; 66%; yellow powder; HPLC purity: 93.7%).

### Example 100

### 2-{[8-(1-Methyl-1H-indol-6-yl)quinoxalin-6-yl]amino}-N-(pyrimidin-5-yl)benzene-1-sulfonamide

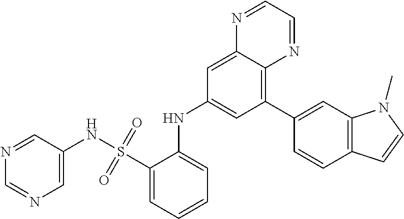 [SMILES: Cn1ccc2ccc(-c3cc(Nc4ccccc4S(=O)(=O)Nc4cncnc4)cc4nccnc34)cc21]

An oven dried microwave vial is charged with copper iodide (3 mg; 0.02 mmol; 0.15 eq.), potassium carbonate (32 mg; 0.23 mmol; 2 eq.) and 2-{[8-(1-methyl-1H-indol-6-yl)quinoxalin-6-yl]amino}benzene-1-sulfonamide (Example 8, 50 mg; 0.12 mmol; 1 eq.), dry DMF (10 mL) (1 S,2S)—N,N′-dimethylcyclohexane-1,2-diamine (5 mg; 0.03 mmol; 0.30 eq.) and 5-bromopyrimidine (22 mg; 0.14 mmol; 1.20 eq.). The resulting blue suspension is stirred at room temperature for 5 min, then heated to 100° C. for 16 h. After coming back to room temperature, the reaction mixture is diluted with EtOAc, washed with water and brine, dried over sodium sulfate and concentrated. The residue is purified by FCC (silica neutralized with ammonia in DCM beforehand, 0% to 100% EtOAc gradient in hexane followed by 0% to 5% MeOH gradient in EtOAc) to afford 2-{[8-(1-methyl-1H-indol-6-yl)quinoxalin-6-yl]amino}-N-(pyrimidin-5-yl)benzene-1-sulfonamide (9 mg; 0.02 mmol; 15%; yellow powder; HPLC purity: 93.8%).

### Example 101

### 3-{[8-(1-Methyl-1H-indol-6-yl)quinoxalin-6-yl]amino}-N-(oxan-4-yl)pyridine-4-carboxamide

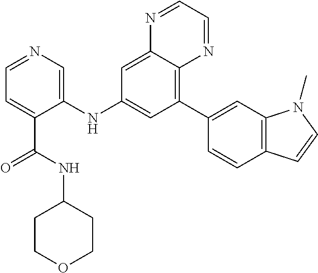 [SMILES: Cn1ccc2ccc(-c3cc(Nc4cnccc4C(=O)NC4CCOCC4)cc4nccnc34)cc21]

3-{[8-(1-methyl-1H-indol-6-yl)quinoxalin-6-yl]amino}pyridine-4-carboxylic acid (Intermediate 42, 50 mg; 0.12 mmol; 1 eq.) is suspended in anhydrous DMF (5 mL) and HATU (68 mg; 0.18 mmol; 1.50 eq.) is added as a solid, and the reaction mixture is stirred at room temperature for 10 min. Tetrahydropyran-4-ylamine (12 mg; 0.12 mmol; 1 eq.) is added and the reaction mixture is stirred at room temperature for 30 min, then N-methylmorpholine (0.04 mL; 0.36 mmol; 3 eq.) is injected by syringe. The reaction mixture is stirred at 70° C. for 16 h. It is then evaporated in vacuo, diluted with DCM (75 mL), filtered through celite, washed with water (4×50 mL), and brine (4×50 mL). The combined aqueous layers are extracted with DCM (3×30 mL) and the combined organic layers are washed with brine (2×30 mL), dried over Na₂SO₄, filtered and concentrated in vacuo. The crude product is purified by FCC (0% to 100% DCM gradient in hexane followed by 0% to 10% MeOH gradient in DCM), to afford 3-{[8-(1-methyl-1H-indol-6-yl)quinoxalin-6-yl]amino}-N-(oxan-4-yl)pyridine-4-carboxamide (44 mg; 0.09 mmol; 72%; yellow solid; HPLC purity: 93.4%).

### Example 102

### 6-Methanesulfonyl-N1-[8-(3-methyl-1-benzofuran-5-yl)quinoxalin-6-yl]benzene-1,3-diamine

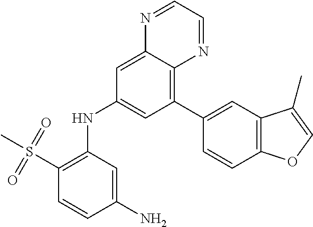 [SMILES: Cc1coc2ccc(-c3cc(Nc4cc(N)ccc4S(C)(=O)=O)cc4nccnc34)cc12]

Raney Nickel (around 20 mg) and hydrazine monohydrate (70 μl; 0.91 mmol; 5 eq) are added to a suspension of N-(2-methanesulfonyl-5-nitrophenyl)-8-(3-methyl-1-benzofuran-5-yl)quinoxalin-6-amine (Example 103, 108 mg; 0.18 mmol; 1 eq.) in EtOH 96% (5 mL). The reaction mixture is left stirring at room temperature for 1 h, diluted with DCM and filtered through a pad of celite. Water is added and the product is extracted with DCM. The organic layer is washed with brine, dried (Na₂SO₄) and concentrated in vacuo. The product is purified by FCC (0% to 100% EtOAc gradient in hexane followed by 0% to 5% MeOH gradient in EtOAc) to 6-methanesulfonyl-N1-[8-(3-methyl-1-benzofuran-5-yl)quinoxalin-6-yl]benzene-1,3-diamine (58 mg; 0.12 mmol; 66%; yellow powder; HPLC purity: 92.2%).

### Example 103

### N-(2-methanesulfonyl-5-nitrophenyl)-8-(3-methyl-1-benzofuran-5-yl)quinoxalin-6-amine

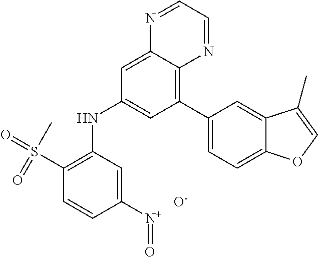

The title compound is prepared according to General Procedure 1 described in Example 1, using 8-(3-methyl-1-benzofuran-5-yl)quinoxalin-6-amine (Example 80, 83 mg; 0.29 mmol; 1 eq.), 2-bromo-1-methanesulfonyl-4-nitrobenzene (90 mg; 0.32 mmol; 1.1 eq.), cesium carbonate (236 mg; 0.72 mmol; 2.5 eq.), BINAP (18 mg; 0.03 mmol; 0.1 eq.), palladium(II) acetate (6.77 mg; 0.03 mmol; 0.1 eq.) and anhydrous dioxane (4 mL). Conditions: 120° C. for 5 hours. Purification by FCC (0% to 100% EtOAc gradient in hexane) affords N-(2-methanesulfonyl-5-nitrophenyl)-8-(3-methyl-1-benzofuran-5-yl)quinoxalin-6-amine (21 mg; 0.04 mmol; 14%; orange powder; HPLC purity: 93.2%).

### Example 104, General Procedure 21

### N-(4-Methanesulfonylpyridin-3-yl)-N-methyl-8-(1-methyl-1H-indol-6-yl)quinoxalin-6-amine

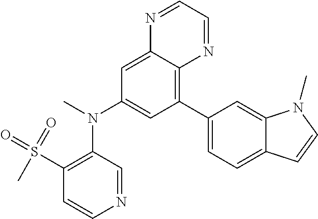 [SMILES: CN(c1cc(-c2ccc3ccn(C)c3c2)c2nccnc2c1)c1cnccc1S(C)(=O)=O]

A solution of N-(4-methanesulfonylpyridin-3-yl)-8-(1-methyl-1H-indol-6-yl)quinoxalin-6-amine (Example 11, 85 mg; 0.19 mmol; 1 eq.) in anhydrous THF (2 mL) is cooled in an ice bath and NaH (60% in mineral oil, 44 mg; 1.12 mmol; 6 eq.) is added. The reaction mixture is stirred at room temperature for 15 min. and iodomethane (40 μl; 0.80 mmol; 4.30 eq.) is added. The reaction mixture is stirred at room temperature overnight, then poured onto ice and extracted twice with EtOAc. The combined organic layers are washed with brine, dried over sodium sulfate and filtered through a pad of celite. The filtrate is concentrated in vacuo and the crude product is purified by FCC (0% to 100% EtOAc gradient in hexane followed by 0% to 2% MeOH gradient in EtOAc) to give N-(4-methanesulfonylpyridin-3-yl)-N-methyl-8-(1-methyl-1H-indol-6-yl)quinoxalin-6-amine (24 mg; 0.05 mmol; 28%; yellow powder; HPLC purity: 95%).

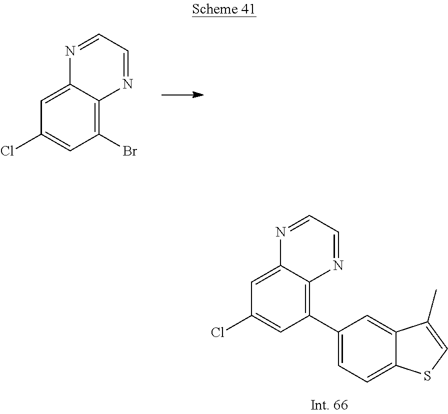 [SMILES: Clc1cc(Br)c2nccnc2c1 ; Cc1csc2ccc(-c3cc(Cl)cc4nccnc34)cc12]

### Intermediate 66

### 7-Chloro-5-(3-methyl-1-benzothiophen-5-yl)quinoxaline

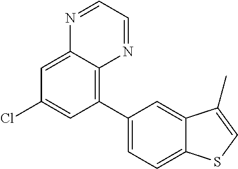 [SMILES: Cc1csc2ccc(-c3cc(Cl)cc4nccnc34)cc12]

The title compound is prepared according to General Procedure 9 described for Intermediate 15, using 5-bromo-7-chloroquinoxaline (Intermediate 2, 350 mg; 1.35 mmol; 1 eq.), 4,4,5,5-tetramethyl-2-(3-methyl-benzo[b]thiophen-5-yl)-[1,3,2]dioxaborolane (399 mg; 1.35 mmol; 1 eq.), cesium carbonate (889 mg; 2.70 mmol; 2 eq.), Pd(dppf)Cl₂.DCM (169 mg; 0.20 mmol; 0.15 eq.) in 1,2-dimethoxyethane (10 mL) and water (5 mL). Conditions: 1 h at 100° C. Purification by FCC (0% to 10% EtOAc gradient in hexane) affords 7-chloro-5-(3-methyl-1-benzothiophen-5-yl)quinoxaline (345 mg; 1.08 mmol; 80%; off-white foam; UPLC purity: 98%).

### Example 110

### N-(4-Methanesulfonylpyridin-3-yl)-8-(3-methyl-1-benzothiophen-5-yl)quinoxalin-6-amine

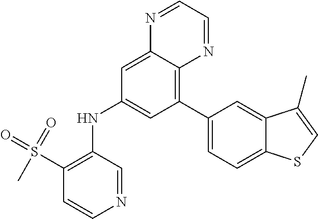 [SMILES: Cc1csc2ccc(-c3cc(Nc4cnccc4S(C)(=O)=O)cc4nccnc34)cc12]

The title compound is prepared according to General Procedure 1 described in Example 1, using 7-chloro-5-(3-methyl-1-benzothiophen-5-yl)quinoxaline (Intermediate 66, 100 mg; 0.31 mmol; 1 eq.), 4-methanesulfonylpyridin-3-ylamine hydrochloride (85 mg; 0.39 mmol; 1.25 eq.), cesium carbonate (512 mg; 1.56 mmol; 5 eq.), BINAP (20 mg; 0.03 mmol; 0.10 eq.) and palladium(II) acetate (7 mg; 0.03 mmol; 0.10 eq.) in dioxane (5 mL). Conditions: 1.5 h at 150° C. Purification by FCC (0% to 100% EtOAc gradient in hexane) affords N-(4-methanesulfonylpyridin-3-yl)-8-(3-methyl-1-benzothiophen-5-yl)quinoxalin-6-amine (108 mg; 0.23 mmol; 73%; pale yellow powder; HPLC purity: 94%).

### Example 113

### N-(1-Methyl-1H-1,2,3-triazol-5-yl)-8-(1-methyl-1H-indol-6-yl)quinoxalin-6-amine

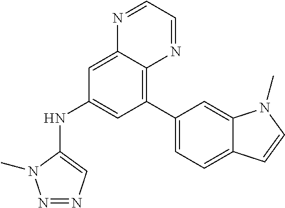 [SMILES: Cn1nncc1Nc1cc(-c2ccc3ccn(C)c3c2)c2nccnc2c1]

The title compound is prepared according to General Procedure 1 described in Example 1, using 7-7-chloro-5-(1-methyl-1H-indol-6-yl)quinoxaline (Intermediate 2B, 103 mg; 0.34 mmol; 1.10 eq.), 3-methyl-3H-[1,2,3]triazol-4-ylamine (30 mg; 0.31 mmol; 1 eq.), cesium carbonate (252 mg; 0.76 mmol; 2.50 eq.), BINAP (20 mg; 0.03 mmol; 0.10 eq.) and palladium(II) acetate (7 mg; 0.03 mmol; 0.10 eq.) in dioxane anhydrous (2 mL). Conditions: 120° C. overnight. Purification by FCC (50% to 100% EtOAc gradient in hexane followed by 0% to 10% MeOH gradient in EtOAc) affords N-(1-methyl-1H-1,2,3-triazol-5-yl)-8-(1-methyl-1H-indol-6-yl)quinoxalin-6-amine (24 mg, 0.06 mmol, 21%; yellow powder; HPLC purity: 93.1%).

### Example 115

### Methyl 4-methanesulfonyl-3-{[8-(1-methyl-1H-indol-6-yl)quinoxalin-6-yl]amino}benzoate

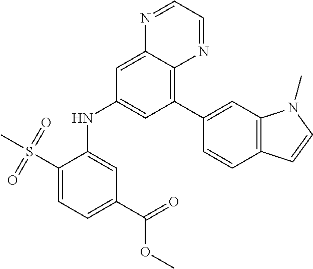 [SMILES: COC(=O)c1ccc(S(C)(=O)=O)c(Nc2cc(-c3ccc4ccn(C)c4c3)c3nccnc3c2)c1]

The title compound is prepared according to General Procedure 1 described in Example 1, using 8-(1-methyl-1H-indol-6-yl)quinoxalin-6-amine (Intermediate 4, 118 mg; 0.41 mmol; 1.20 eq.), 3-bromo-4-methanesulfonylbenzoic acid methyl ester (100 mg; 0.34 mmol; 1 eq.), cesium carbonate (157 mg; 0.48 mmol; 1.40 eq.), BINAP (17 mg; 0.03 mmol; 0.08 eq.) and palladium(II) acetate (4 mg; 0.02 mmol; 0.05 eq.) in toluene (3 mL). Conditions: 1 h at 120° C. Purification by FCC (30% to 70% EtOAc gradient in hexane) gives methyl 4-methanesulfonyl-3-{[8-(1-methyl-1H-indol-6-yl)quinoxalin-6-yl]amino}benzoate (122 mg; 0.25 mmol; 73%; pale yellow powder; HPLC purity: 99.4%).

### Example 116

### 4-Methanesulfonyl-3-{[8-(1-methyl-1H-indol-6-yl)quinoxalin-6-yl]amino}benzamide

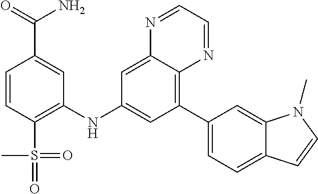 [SMILES: Cn1ccc2ccc(-c3cc(Nc4cc(C(N)=O)ccc4S(C)(=O)=O)cc4nccnc34)cc21]

In a pressure vessel, 25% aqueous ammonia (1 mL; 6.49 mmol; 67 eq.) is added to a solution of methyl 4-methanesulfonyl-3-{[8-(1-methyl-1H-indol-6-yl)quinoxalin-6-yl]amino}benzoate (Example 115, 50 mg; 0.10 mmol; 1 eq.) in anhydrous ethanol (2 mL). The vessel is sealed and the reaction mixture is stirred overnight at 120° C. After coming back to room temperature, it is evaporated to dryness and the residue is purified by FCC (0% to 10% MeOH gradient in DCM) to afford 4-methanesulfonyl-3-{[8-(1-methyl-1H-indol-6-yl)quinoxalin-6-yl]amino}benzamide (17 mg; 0.04 mmol; 37%; yellow solid; HPLC purity: 99%).

### Example 117

### 8-(2,1,3-Benzothiadiazol-5-yl)-N-(4-methanesulfonylpyridin-3-yl)quinoxalin-6-amine

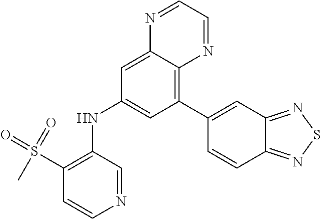 [SMILES: CS(=O)(=O)c1ccncc1Nc1cc(-c2ccc3nsnc3c2)c2nccnc2c1]

The title compound is prepared according to General Procedure 15 described in Example 60, using 5-(4,4,5,5-tetramethyl-[1,3,2]dioxaborolan-2-yl)-benzo[1,2,5]thiadiazole (30 mg; 0.12 mmol; 1.20 eq.), 8-chloro-N-(4-methanesulfonylpyridin-3-yl)quinoxalin-6-amine (Intermediate 3B, 33 mg; 0.10 mmol; 1 eq.), sodium carbonate (15 mg; 0.15 mmol; 1.50 eq.), tetrakis(triphenylphosphine)palladium(0) (11 mg, 0.01 mmol, 0.1 eq.) in dioxane (1.3 mL) and water (1.3 mL). Conditions: 140° C. under microwave irradiation for 90 min. Purification by reversed-phase preparative HPLC (column: Gemini NX C18 5u 110A (100×30 mm), ACN gradient in water) yielded 8-(2,1,3-benzothiadiazol-5-yl)-N-(4-methanesulfonylpyridin-3-yl)quinoxalin-6-amine formate salt (5 mg, 0.01 mmol, 11%; yellow powder; HPLC purity: 100%).

### Example 118

### 8-(1H-1,2,3-Benzotriazol-5-yl)-N-(4-methanesulfonylpyridin-3-yl)quinoxalin-6-amine

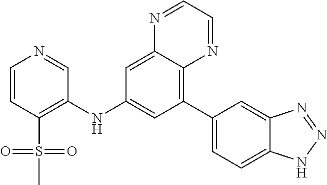 [SMILES: CS(=O)(=O)c1ccncc1Nc1cc(-c2ccc3[nH]nnc3c2)c2nccnc2c1]

The title compound is prepared according to General Procedure 17 described in Example 66, using 8-chloro-N-(4-methanesulfonylpyridin-3-yl)quinoxalin-6-amine (Intermediate 3B, 40 mg; 0.12 mmol; 1 eq.), 5-(4,4,5,5-tetramethyl-[1,3,2]dioxaborolan-2-yl)-1H-benzotriazole (96 mg; 0.28 mmol; 2.4 eq.), sodium carbonate (34 mg; 0.32 mmol; 2.6 eq.), tetrakis(triphenylphosphine)-palladium(0) (12 mg; 0.01 mmol; 0.09 eq.) in 1.4-dioxane (1 mL) and water (2 mL). Conditions: 130° C. under microwave irradiation for 2 h. Purification by FCC (0% to 100% EtOAc gradient in hexane) and preparative HPLC yielded 8-(1H-1,2,3-benzotriazol-5-yl)-N-(4-methanesulfonylpyridin-3-yl)quinoxalin-6-amine (5 mg, 0.01 mmol, 9%; yellow powder; HPLC purity: 100%).

### Example 119

### 4-Methanesulfonyl-3-{[8-(1-methyl-1H-indol-6-yl)quinoxalin-6-yl]amino}benzohydrazide

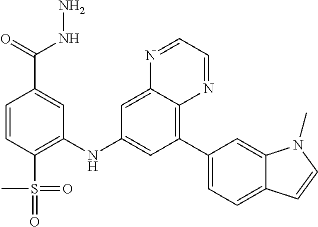 [SMILES: Cn1ccc2ccc(-c3cc(Nc4cc(C(=O)NN)ccc4S(C)(=O)=O)cc4nccnc34)cc21]

In a pressure vessel, hydrazine hydrate (28.35 μl; 0.35 mmol; 3 eq.) is added to a solution of methyl 4-methanesulfonyl-3-{[8-(1-methyl-1H-indol-6-yl)quinoxalin-6-yl]amino}benzoate (Example 115, 60 mg; 0.12 mmol; 1 eq.) in anhydrous ethanol (2 mL). The vessel is sealed and the reaction mixture is stirred overnight at 80° C. After coming back to room temperature, it is evaporated to dryness and the residue is purified by FCC (0% to 10% MeOH gradient in DCM) to afford 4-methanesulfonyl-3-{[8-(1-methyl-1H-indol-6-yl)quinoxalin-6-yl]amino}benzohydrazide (4 mg; 0.01 mmol; 7%; yellow amorphous powder; HPLC purity: 95.2%).

### Example 120

### 8-(2,1,3-Benzoxadiazol-5-yl)-N-(4-methanesulfonylpyridin-3-yl)quinoxalin-6-amine

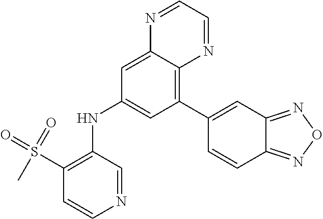 [SMILES: CS(=O)(=O)c1ccncc1Nc1cc(-c2ccc3nonc3c2)c2nccnc2c1]

The title compound is prepared according to General Procedure 17 described in Example 66, using 8-chloro-N-(4-methanesulfonylpyridin-3-yl)quinoxalin-6-amine (Intermediate 3B, 50 mg; 0.15 mmol; 1 eq.), 5-(4,4,5,5-tetramethyl-[1,3,2]dioxaborolan-2-yl)-benzo[1,2,5]oxadiazole (43 mg; 0.18 mmol; 1.20 eq.), sodium carbonate (23 mg; 0.22 mmol; 1.50 eq.) and tetrakis(triphenyl-phosphine)palladium(0) (17 mg; 0.01 mmol; 0.10 eq.) in dioxane (1.4 mL) and water (1.4 mL). Conditions: 140° C. under microwave irradiation for 105 min. Purification by FCC (0-75% EtOAc gradient in hexane) and preparative HPLC yielded 8-(2,1,3-benzoxadiazol-5-yl)-N-(4-methanesulfonylpyridin-3-yl)quinoxalin-6-amine (10 mg, 0.02 mmol, 16%; yellow powder; HPLC purity: 100%).

### Example 121

### N-(1-Acetylpyrrolidin-3-yl)-3-{[8-(1-methyl-1H-indol-6-yl)quinoxalin-6-yl]amino}pyridine-4-carboxamide

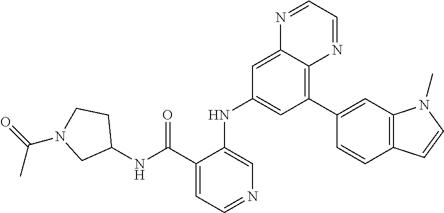 [SMILES: CC(=O)N1CCC(NC(=O)c2ccncc2Nc2cc(-c3ccc4ccn(C)c4c3)c3nccnc3c2)C1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 50 mg; 0.12 mmol; 1 eq.), 1-(3-Amino-pyrrolidin-1-yl)-ethanone (20 mg; 0.15 mmol; 1.25 eq.), EDC.HCl (44 mg; 0.22 mmol; 1.80 eq.), HOBt hydrate (35 mg; 0.22 mmol; 1.80 eq.), triethylamine (0.08 mL; 0.62 mmol; 5 eq.) and dioxane (5 mL). Conditions: room temperature for 24 h. Purification by FCC (column: PF—NH2/30 um/6G, 0% to 2% MeOH gradient in DCM) affords N-(1-acetylpyrrolidin-3-yl)-3-{[8-(1-methyl-1H-indol-6-yl)quinoxalin-6-yl]amino}pyridine-4-carboxamide (56 mg; 0.11 mmol; 86%; yellow powder; HPLC purity: 96.8%).

### Example 122

### 3-{[8-(1-Methyl-1H-indol-6-yl)quinoxalin-6-yl]amino}-N-(1-methyl-6-oxopiperidin-3-yl)pyridine-4-carboxamide

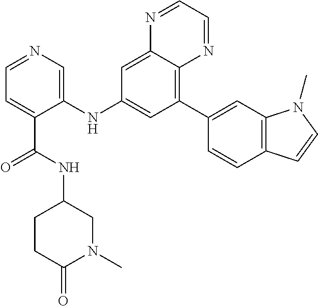 [SMILES: CN1CC(NC(=O)c2ccncc2Nc2cc(-c3ccc4ccn(C)c4c3)c3nccnc3c2)CCC1=O]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 45 mg; 0.11 mmol; 1 eq.), 5-Amino-1-methylpiperidin-2-one (19 mg; 0.14 mmol; 1.25 eq.), EDC.HCl (39 mg; 0.20 mmol; 1.80 eq.), HOBt hydrate (31 mg; 0.20 mmol; 1.80 eq.), triethylamine (0.07 mL; 0.56 mmol; 5 eq.) and dioxane (5 mL). Conditions: room temperature for 24 h. Purification by FCC (column: PF—NH2/30 um/6G, 0% to 2% MeOH gradient in DCM) affords 3-{[8-(1-methyl-1H-indol-6-yl)quinoxalin-6-yl]amino}-N-(1-methyl-6-oxopiperidin-3-yl)pyridine-4-carboxamide (48 mg; 0.09 mmol; 83%; yellow powder; HPLC purity: 98.1%).

### Example 123

### 3-{[8-(1-Methyl-1H-indol-6-yl)quinoxalin-6-yl]amino}-N-(1-methylpiperidin-4-yl)pyridine-4-carboxamide

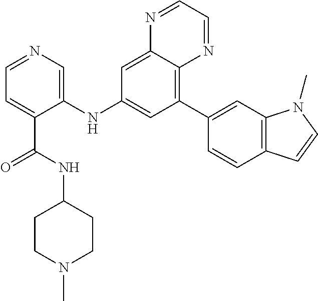 [SMILES: CN1CCC(NC(=O)c2ccncc2Nc2cc(-c3ccc4ccn(C)c4c3)c3nccnc3c2)CC1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 45 mg; 0.11 mmol; 1 eq.), 1-methylpiperidin-4-ylamine (16 mg; 0.14 mmol; 1.25 eq.), EDC.HCl (39 mg; 0.20 mmol; 1.80 eq.), HOBt hydrate (31 mg; 0.20 mmol; 1.80 eq.), triethylamine (0.07 mL; 0.56 mmol; 5 eq.) and dioxane (5 mL). Conditions: room temperature for 24 h. Purification by FCC (0% to 20% MeOH gradient in DCM) affords 3-{[8-(1-methyl-1H-indol-6-yl)quinoxalin-6-yl]amino}-N-(1-methylpiperidin-4-yl)pyridine-4-carboxamide (52 mg; 0.10 mmol; 92%; yellow powder; HPLC purity: 97.2%).

### Example 124

### 3-{[8-(1-Methyl-1H-indol-6-yl)quinoxalin-6-yl]amino}-N-(1-methylpiperidin-3-yl)pyridine-4-carboxamide

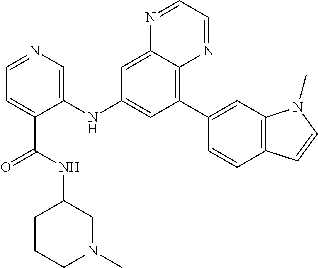 [SMILES: CN1CCCC(NC(=O)c2ccncc2Nc2cc(-c3ccc4ccn(C)c4c3)c3nccnc3c2)C1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 45 mg; 0.11 mmol; 1 eq.), 1-methylpiperidin-3-ylamine hydrochloride (26.89 mg; 0.14 mmol; 1.25 eq.), EDC.HCl (39 mg; 0.20 mmol; 1.80 eq.), HOBt hydrate (31 mg; 0.20 mmol; 1.80 eq.), triethylamine (0.07 mL; 0.56 mmol; 5 eq.) and dioxane (5 mL). Conditions: room temperature for 24 h. Purification by FCC (0% to 10% MeOH gradient in DCM) affords 3-{[8-(1-methyl-1H-indol-6-yl)quinoxalin-6-yl]amino}-N-(1-methylpiperidin-3-yl)pyridine-4-carboxamide (51 mg; 0.10 mmol; 89%; yellow powder; HPLC purity: 95.7%).

### Example 125

### 3-{Methyl[8-(1-methyl-1H-indol-6-yl)quinoxalin-6-yl]amino}-N-(pyrimidin-5-yl)pyridine-4-carboxamide

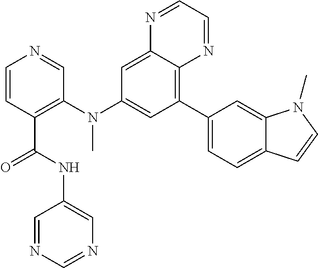 [SMILES: CN(c1cc(-c2ccc3ccn(C)c3c2)c2nccnc2c1)c1cnccc1C(=O)Nc1cncnc1]

A microwave vial is charged with 3-{methyl[8-(1-methyl-1H-indol-6-yl)quinoxalin-6-yl]amino}pyridine-4-carboxamide (Example 95, 18 mg; 0.04 mmol; 1 eq.), potassium phosphate tribasic (11 mg; 0.05 mmol; 1.20 eq.), Pd₂(dba)₃ (2 mg; 2 μmol; 0.05 eq.) and Me₄tBuXPhos (5 mg; 0.01 mmol; 0.25 eq.). The tube is sealed with a septum cap, evacuated and backfilled with argon (three times) and a solution of 5-bromopyrimidine (10 mg; 0.06 mmol; 1.50 eq.) in tert-butanol (1 mL) is added. The vial is sealed and the reaction mixture is stirred at 110° C. for 48 h. After coming back to room temperature, it is filtered through a celite pad and the filtrate is diluted with EtOAc. The solution is washed with water, brine, dried over Na₂SO₄ and concentrated in vacuo. The crude product is purified by FCC (0% to 100% EtOAc gradient in hexane followed by 0% to 10% MeOH gradient in EtOAc) to yield 3-{methyl[8-(1-methyl-1H-indol-6-yl)quinoxalin-6-yl]amino}-N-(pyrimidin-5-yl)pyridine-4-carboxamide (19 mg; 0.04 mmol; 89%; yellow powder; HPLC purity: 95.2%).

### Example 126, General Procedure 22

### N-Cyclohexyl-3-{[8-(1-methyl-1H-indol-6-yl)quinoxalin-6-yl]amino}pyridine-4-carboxamide

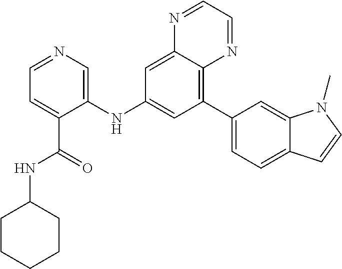 [SMILES: Cn1ccc2ccc(-c3cc(Nc4cnccc4C(=O)NC4CCCCC4)cc4nccnc34)cc21]

3-{[8-(1-Methyl-1H-indol-6-yl)quinoxalin-6-yl]amino}pyridine-4-carboxylic acid (Intermediate 42, 75 mg; 0.18 mmol; 1 eq.) is suspended in anhydrous DMF (5 mL) with stirring and HATU (87 mg; 0.23 mmol; 1.50 eq.) is added as a solid followed by DIPEA (0.13 mL; 0.92 mmol; 5 eq.). The reaction mixture is stirred at room temperature for 10 min and cyclohexylamine (18 mg; 0.18 mmol; 1 eq.) is added. The reaction mixture is stirred at 50° C. for 72 h, evaporated to dryness, diluted with DCM (100 mL), and filtered through Celite. The filtrate is washed with water (6×20 mL) and brine (3×30 mL). The combined aqueous layers are extracted with DCM (3×30 mL) and the combined organic layers are washed with brine (3×30 mL), dried over anhydrous Na₂SO₄, filtered and concentrated under reduced pressure. The crude product is purified by FCC (0% to 5% MeOH gradient in DCM) to afford N-cyclohexyl-3-{[8-(1-methyl-1H-indol-6-yl)quinoxalin-6-yl]amino}pyridine-4-carboxamide (69 mg; 0.14 mmol; 77%; yellow solid; HPLC purity: 98.3%).

### Example 127

### 3-{[8-(1-methyl-1H-indol-6-yl)quinoxalin-6-yl]amino}-N-(2-oxopiperidin-4-yl)pyridine-4-carboxamide

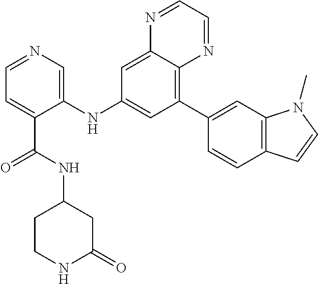 [SMILES: Cn1ccc2ccc(-c3cc(Nc4cnccc4C(=O)NC4CCNC(=O)C4)cc4nccnc34)cc21]

The title compound is prepared according to General Procedure 22 described in Example 126, using 3-{[8-(1-methyl-1H-indol-6-yl)quinoxalin-6-yl]amino}pyridine-4-carboxylic acid (Intermediate 42, 75 mg; 0.18 mmol; 1 eq.), HATU (105 mg; 0.28 mmol; 1.50 eq.), DIPEA (0.21 mL; 1.47 mmol; 8 eq.), 4-aminopiperidin-2-one trifluoroacetate (42 mg; 0.18 mmol; 1 eq.) in anhydrous DMF (5 mL). Conditions: 50° C. for 72 h. Purification by FCC (0% to 5% MeOH gradient in DCM) affords 3-{[8-(1-methyl-1H-indol-6-yl)quinoxalin-6-yl]amino}-N-(2-oxopiperidin-4-yl)pyridine-4-carboxamide (75 mg; 0.15 mmol; 81%; yellow powder; HPLC purity: 98.3%).

### Example 128

### 2-{7-[(4-methanesulfonylpyridin-3-yl)amino]quinoxalin-5-yl}-4-methylbenzamide

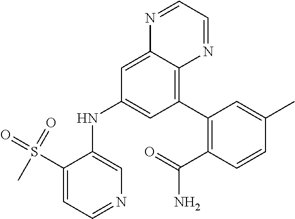 [SMILES: Cc1ccc(C(N)=O)c(-c2cc(Nc3cnccc3S(C)(=O)=O)cc3nccnc23)c1]

The title compound is prepared according to General Procedure 16 described in Example 62, using 8-chloro-N-(4-methanesulfonylpyridin-3-yl)quinoxalin-6-amine (Intermediate 3B, 60 mg; 0.18 mmol; 1 eq.), 4-methyl-2-(4,4,5,5-tetramethyl-[1,3,2]dioxaborolan-2-yl)-benzonitrile (52 mg; 0.22 mmol; 1.20 eq.), cesium carbonate (175 mg; 0.54 mmol; 3 eq.), tetrakis(triphenyl-phosphine)palladium(0) (21 mg; 0.02 mmol; 0.10 eq.) in dioxane (2 mL) and water (1 mL). Conditions: 100° C. for 4 h. Purification by FCC (0% to 5% MeOH gradient in DCM) yields 2-{7-[(4-methanesulfonylpyridin-3-yl)amino]quinoxalin-5-yl}-4-methylbenzamide (42 mg; 0.09 mmol; 51%; yellow powder; HPLC purity: 94.1%).

### Example 129

### 8-(3-Ethoxyphenyl)-N-(4-methanesulfonylpyridin-3-yl)quinoxalin-6-amine

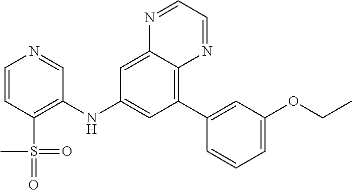 [SMILES: CCOc1cccc(-c2cc(Nc3cnccc3S(C)(=O)=O)cc3nccnc23)c1]

The title compound is prepared according to General Procedure 17 described in Example 66, using chloro-N-(4-methanesulfonylpyridin-3-yl)quinoxalin-6-amine (Intermediate 3B, 75 mg; 0.22 mmol; 1 eq.), sodium carbonate (115 mg; 1.09 mmol; 5 eq.), (3-ethoxyphenyl)boronic acid (40 mg; 0.24 mmol; 1.10 eq.), tetrakis(triphenylphosphine)palladium(0) (13 mg; 0.01 mmol; 0.05 eq.) in water (0.50 mL), ethanol (0.50 mL) and toluene (1 mL). Conditions: 110° C. overnight. Purification by FCC (50% to 75% EtOAc gradient in hexane) yields 8-(3-ethoxyphenyl)-N-(4-methanesulfonylpyridin-3-yl)quinoxalin-6-amine (38 mg; 0.09 mmol; 41%; yellow powder; HPLC purity: 99.5%).

### Example 130

### N-(4-Methanesulfonylpyridin-3-yl)-8-[3-(propan-2-yloxy)phenyl]quinoxalin-6-amine

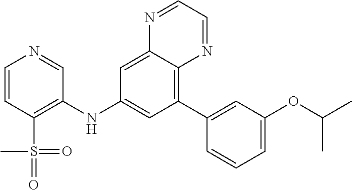 [SMILES: CC(C)Oc1cccc(-c2cc(Nc3cnccc3S(C)(=O)=O)cc3nccnc23)c1]

The title compound is prepared according to General Procedure 17 described in Example 66, using chloro-N-(4-methanesulfonylpyridin-3-yl)quinoxalin-6-amine (Intermediate 3B, 75 mg; 0.22 mmol; 1 eq.), sodium carbonate (115 mg; 1.09 mmol; 5 eq.), (3-isopropoxyphenyl)boronic acid (43 mg; 0.24 mmol; 1.10 eq.), tetrakis(triphenylphosphine)palladium(0) (13 mg; 0.01 mmol; 0.05 eq.) in water (0.50 mL), ethanol (0.50 mL) and toluene (1 mL). Conditions: 110° C. overnight. Purification by FCC (50% to 75% EtOAc gradient in hexane) yields N-(4-methanesulfonylpyridin-3-yl)-8-[3-(propan-2-yloxy)phenyl]quinoxalin-6-amine (24 mg; 0.05 mmol; 25%; yellow powder; HPLC purity: 99.2%).

### Example 131

### 8-(4-Aminophenyl)-N-(4-methanesulfonylpyridin-3-yl)quinoxalin-6-amine

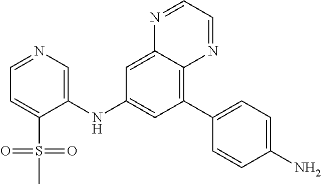 [SMILES: CS(=O)(=O)c1ccncc1Nc1cc(-c2ccc(N)cc2)c2nccnc2c1]

The title compound is prepared according to General Procedure 17 described in Example 66, using chloro-N-(4-methanesulfonylpyridin-3-yl)quinoxalin-6-amine (Intermediate 3B, 150 mg; 0.43 mmol; 1 eq.), sodium carbonate (230 mg; 2.17 mmol; 5 eq.), tetramethyl-[1,3,2]dioxaborolan-2-yl)-phenylamine (105 mg; 0.48 mmol; 1.10 eq.), tetrakis(triphenylphosphine)palladium(0) (26 mg; 0.02 mmol; 0.05 eq.) in water (1 mL), ethanol (1 mL) and toluene (2 mL). Conditions: 110° C. overnight. Purification by FCC (40% to 60% acetone gradient in hexane) affords 8-(4-aminophenyl)-N-(4-methanesulfonylpyridin-3-yl)quinoxalin-6-amine (110 mg; 0.28 mmol; 64%; yellow powder; HPLC purity: 99.6%).

### Example 132

### 8-(3-Aminophenyl)-N-(4-methanesulfonylpyridin-3-yl)quinoxalin-6-amine

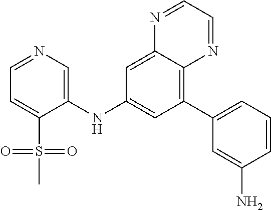 [SMILES: CS(=O)(=O)c1ccncc1Nc1cc(-c2cccc(N)c2)c2nccnc2c1]

The title compound is prepared according to General Procedure 17 described in Example 66, using chloro-N-(4-methanesulfonylpyridin-3-yl)quinoxalin-6-amine (Intermediate 3B, 150 mg; 0.43 mmol; 1 eq.), sodium carbonate (230 mg; 2.17 mmol; 5 eq.), 3-aminophenyl boronic acid (105 mg; 0.48 mmol; 1.10 eq.), tetrakis(triphenylphosphine)palladium(0) (26 mg; 0.02 mmol; 0.05 eq.) in water (1 mL), ethanol (1 mL) and toluene (2 mL). Conditions: 110° C. overnight. Purification by FCC (40% to 60% acetone gradient in hexane) affords 8-(3-aminophenyl)-N-(4-methanesulfonylpyridin-3-yl)quinoxalin-6-amine (150 mg; 0.38 mmol; 87%; yellow powder; HPLC purity: 98.8%).

### Example 135

### 3-{[8-(1-Methyl-1H-indol-6-yl)quinoxalin-6-yl]amino}-N-[(morpholin-3-yl)methyl]pyridine-4-carboxamide

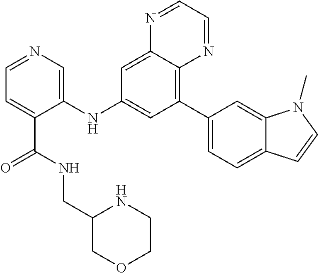 [SMILES: Cn1ccc2ccc(-c3cc(Nc4cnccc4C(=O)NCC4COCCN4)cc4nccnc34)cc21]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 70 mg; 0.18 mmol; 1 eq.), 3-aminomethylmorpholine-4-carboxylic acid tert-butyl ester (44 μl; 0.22 mmol; 1.25 eq.), EDC.HCl (68 mg; 0.3 mmol; 1.70 eq.), HOBt hydrate (46 mg; 0.3 mmol; 1.70 eq.), triethylamine (0.11 mL; 0.89 mmol; 5 eq.) in dioxane (5 mL). Conditions: 2 days at room temperature. Purification by FCC (column: PF—NH2/30 um/6G, 0% to 10% MeOH gradient in DCM) yields tea-butyl 3-{[(3-{[8-(1-methyl-1H-indol-6-yl)quinoxalin-6-yl]amino}pyridin-4-yl)formamido]methyl}morpholine-4-carboxylate which is subsequently deprotected in DCM/TFA (4 mL) for 30 minutes at room temperature. After evaporation of the solvent, the crude material is purified by reversed-phase preparative HPLC (column: Gemini NX C18 5u 110A (100×30 mm), ACN gradient in water) to yield 3-{[8-(1-methyl-1H-indol-6-yl)quinoxalin-6-yl]amino}-N-[(morpholin-3-yl)methyl]pyridine-4-carboxamide formate salt (53 mg; 0.08 mmol; 46%; yellow powder; HPLC purity: 96%).

### Example 136

### N-[(4-acetylmorpholin-3-yl)methyl]-3-{[8-(1-methyl-1H-indol-6-yl)quinoxalin-6-yl]amino}pyridine-4-carboxamide

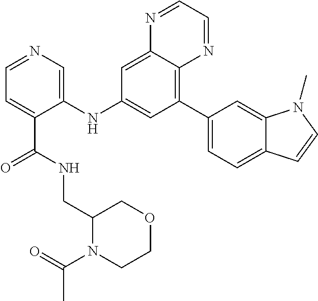 [SMILES: CC(=O)N1CCOCC1CNC(=O)c1ccncc1Nc1cc(-c2ccc3ccn(C)c3c2)c2nccnc2c1]

Acetic anhydride (16 μl; 0.17 mmol; 1.10 eq.) is added to a solution of 3-{[8-(1-methyl-1H-indol-6-yl)quinoxalin-6-yl]amino}-N-[(morpholin-3-yl)methyl]pyridine-4-carboxamide formate salt (Example 135, 75 mg; 0.15 mmol; 1 eq.) and triethylamine (49 μl; 0.38 mmol; 2.50 eq.) in anhydrous DCM (10 mL). The reaction mixture is stirred at room temperature for 1 h, quenched with saturated aqueous NaHCO₃, and extracted with n-butanol. The solvent is evaporated in vacuo and the residue is purified by reversed-phase preparative HPLC (column: Gemini NX C18 5u 110A (100×30 mm), ACN gradient in water) to yield N-[(4-acetylmorpholin-3-yl)methyl]-3-{[8-(1-methyl-1H-indol-6-yl)quinoxalin-6-yl]amino}pyridine-4-carboxamide formate salt (10 mg; 0.02 mmol; 11.4%; yellow powder; HPLC purity: 96.3%).

### Example 137

### 3-{[8-(1-methyl-1H-indol-6-yl)quinoxalin-6-yl]amino}-N-[(4-methylmorpholin-2-yl)methyl]pyridine-4-carboxamide

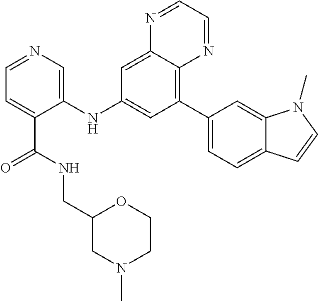 [SMILES: CN1CCOC(CNC(=O)c2ccncc2Nc2cc(-c3ccc4ccn(C)c4c3)c3nccnc3c2)C1]

The title compound is prepared according to General Procedure 22 using 3-{[8-(1-methyl-1H-indol-6-yl)quinoxalin-6-yl]amino}pyridine-4-carboxylic acid (Intermediate 42, 75 mg; 0.18 mmol; 1 eq.), HATU (105 mg; 0.28 mmol; 1.50 eq.), (4-methylmorpholin-2-yl)methanamine (24 mg; 0.18 mmol; 1 eq.) and DIPEA (0.08 mL; 0.55 mmol; 3 eq.) in anhydrous DMF (5 mL). Conditions: room temperature for 48 h. Purification by FCC (0% to 100% DCM gradient in hexane followed by 0% to 10% MeOH gradient in DCM) affords 3-{[8-(1-methyl-1H-indol-6-yl)quinoxalin-6-yl]amino}-N-[(4-methylmorpholin-2-yl)methyl]pyridine-4-carboxamide (59 mg; 0.11 mmol; 62%; yellow solid; HPLC purity: 98.2%).

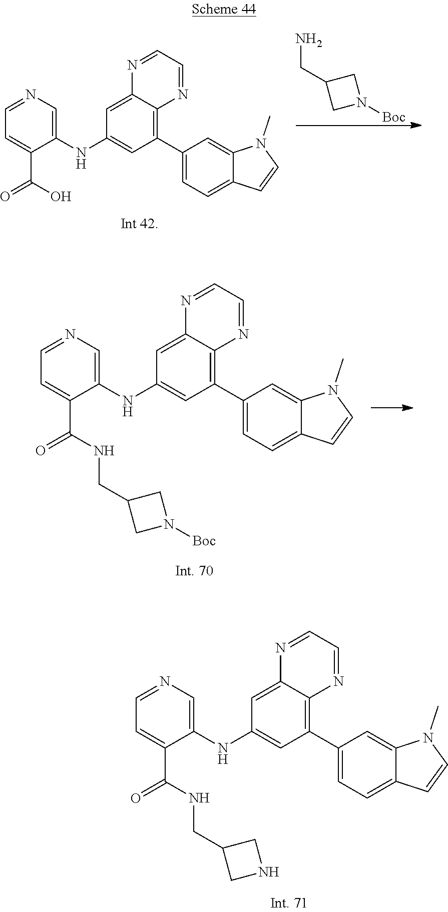 [SMILES: Cn1ccc2ccc(-c3cc(Nc4cnccc4C(=O)O)cc4nccnc34)cc21 ; CC(C)(C)OC(=O)N1CC(CN)C1 ; Cn1ccc2ccc(-c3cc(Nc4cnccc4C(=O)NCC4CN(C(=O)OC(C)(C)C)C4)cc4nccnc34)cc21 ; Cn1ccc2ccc(-c3cc(Nc4cnccc4C(=O)NCC4CNC4)cc4nccnc34)cc21]

### Intermediate 70

### tert-Butyl 3-{[(3-{[8-(1-methyl-1H-indol-6-yl)quinoxalin-6-yl]amino}pyridin-4-yl)formamido]methyl}azetidine-1-carboxylate

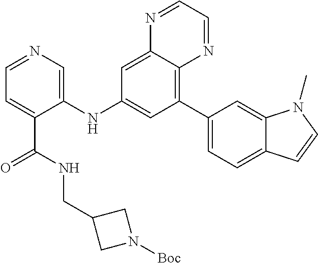 [SMILES: Cn1ccc2ccc(-c3cc(Nc4cnccc4C(=O)NCC4CN(C(=O)OC(C)(C)C)C4)cc4nccnc34)cc21]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 100 mg; 0.25 mmol; 1 eq), EDC.HCl (82 mg; 0.43 mmol; 1.70 eq.), HOBt hydrate (66 mg; 0.43 mmol; 1.70 eq.), triethylamine (0.16 mL; 1.26 mmol; 5 eq.), aminomethylazetidine-1-carboxylic acid tert-butyl ester (55 μl; 0.32 mmol; 1.25 eq.) in dioxane (5 mL). Conditions: room temperature for 2 days. Purification by FCC (column: PF—NH2/30 um/6G, 0% to 10% MeOH gradient in DCM) affords tert-butyl 3-{[(3-{[8-(1-methyl-1H-indol-6-yl)quinoxalin-6-yl]amino}pyridin-4-yl)formamido]methyl}azetidine-1-carboxylate (94 mg; 0.16 mmol; 65%; yellow powder; UPLC purity: 98%).

### Intermediate 71

### N-(Azetidin-3-ylmethyl)-3-{[8-(1-methyl-1H-indol-6-yl)quinoxalin-6-yl]amino}pyridine-4-carboxamide

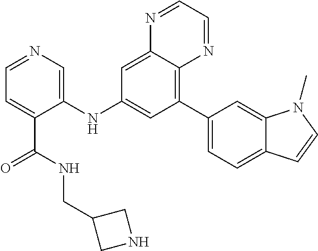 [SMILES: Cn1ccc2ccc(-c3cc(Nc4cnccc4C(=O)NCC4CNC4)cc4nccnc34)cc21]

3-{[(3-{[8-(1-Methyl-1H-indol-6-yl)quinoxalin-6-yl]amino}pyridin-4-yl)formamido]methyl}azetidine-1-carboxylate (Intermediate 70, 94 mg; 0.17 mmol; 1 eq.) is dissolved in DCM (3 mL) and TFA (1 mL) is added. The reaction mixture is stirred 30 min at room temperature and evaporated. The residue is dissolved in DCM and vigorously stirred with aqueous saturated NaHCO₃ for 5 minutes. The layers are separated and the aqueous layer is extracted with DCM. The combined organic layers are dried over sodium sulfate, filtered and evaporated in vacuo. The crude N-(azetidin-3-ylmethyl)-3-{[8-(1-methyl-1H-indol-6-yl)quinoxalin-6-yl]amino}pyridine-4-carboxamide (75 mg; 0.16 mmol; 97%; yellow powder; UPLC purity: 99%) is used in the next step without further purification.

### Example 140

### N-[(1-Acetylazetidin-3-yl)methyl]-3-{[8-(1-methyl-1H-indol-6-yl)quinoxalin-6-yl]amino}pyridine-4-carboxamide

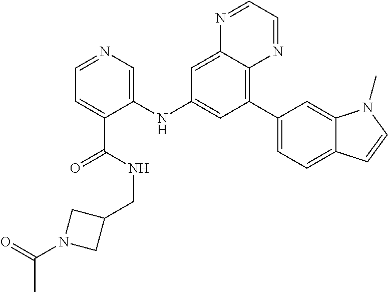 [SMILES: CC(=O)N1CC(CNC(=O)c2ccncc2Nc2cc(-c3ccc4ccn(C)c4c3)c3nccnc3c2)C1]

Acetic anhydride (17 μL; 0.18 mmol; 1.10 eq.) is added to a solution of N-(azetidin-3-ylmethyl)-3-{[8-(1-methyl-1H-indol-6-yl)quinoxalin-6-yl]amino}pyridine-4-carboxamide (Intermediate 71, 75 mg; 0.16 mmol; 1 eq.) and triethylamine (52 μL; 0.40 mmol; 2.50 eq.) in anhydrous DCM (10 mL) and the reaction mixture is stirred at room temperature for 1 h. The reaction is quenched with saturated aqueous NaHCO₃, and extracted with t-butanol. The solvent is evaporated in vacuo and the residue is purified by reversed-phase preparative HPLC (column: Gemini NX C18 5u 110A (100×30 mm), ACN gradient in water) to yield N-[(1-acetylazetidin-3-yl)methyl]-3-{[8-(1-methyl-1H-indol-6-yl)quinoxalin-6-yl]amino}pyridine-4-carboxamide (20 mg; 0.04 mmol; 24%; yellow powder; HPLC purity: 99.6%).

### Example 141

### N-[(4-Acetylmorpholin-2-yl)methyl]-3-{[8-(1-methyl-1H-indol-6-yl)quinoxalin-6-yl]amino}pyridine-4-carboxamide

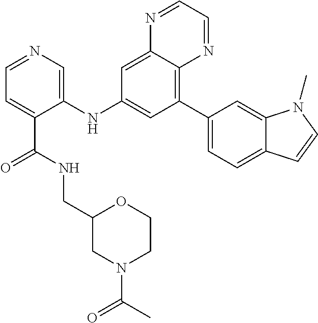 [SMILES: CC(=O)N1CCOC(CNC(=O)c2ccncc2Nc2cc(-c3ccc4ccn(C)c4c3)c3nccnc3c2)C1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 50 mg; 0.13 mmol; 1 eq.), EDC.HCl (29 mg; 0.15 mmol; 1.20 eq.), HOBt hydrate (23 mg; 0.15 mmol; 1.20 eq.), 1-(2-aminomethylmorpholin-4-yl)-ethanone hydrochloride (33 μl; 0.16 mmol; 1.25 eq.), triethylamine (0.08 mL; 0.63 mmol; 5 eq.) in dioxane (5 mL). Conditions: overnight at room temperature. Purification by reversed-phase preparative HPLC (column: Gemini NX C18 5u 110A (100×30 mm), ACN gradient in water) yielded N-[(4-acetylmorpholin-2-yl)methyl]-3-{[8-(1-methyl-1H-indol-6-yl)quinoxalin-6-yl]amino}pyridine-4-carboxamide (15 mg; 0.03 mmol; 22%; yellow powder; HPLC purity: 100%).

### Example 142

### 3-{[8-(1-Methyl-1H-indol-6-yl)quinoxalin-6-yl]amino}-N-[(1-methylpyrrolidin-3-yl)methyl]pyridine-4-carboxamide

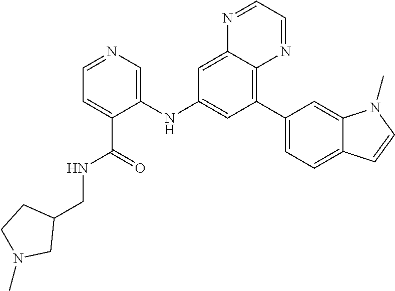 [SMILES: CN1CCC(CNC(=O)c2ccncc2Nc2cc(-c3ccc4ccn(C)c4c3)c3nccnc3c2)C1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 45 mg; 0.11 mmol; 1 eq.), (1-methylpyrrolidin-3-yl)methanamine (17 mg; 0.14 mmol; 1.25 eq.), EDC.HCl (39 mg; 0.20 mmol; 1.80 eq.), HOBt hydrate (31 mg; 0.20 mmol; 1.80 eq.), triethylamine (0.07 mL; 0.56 mmol; 5 eq.) and dioxane (5 mL). Conditions: room temperature for 48 h. Purification by FCC (column: PF—NH2/30 um/6G, 0% to 2% MeOH gradient in DCM) affords 3-{[8-(1-methyl-1H-indol-6-yl)quinoxalin-6-yl]amino}-N-[(1-methylpyrrolidin-3-yl)methyl]pyridine-4-carboxamide (27 mg; 0.05 mmol; 45%; yellow powder; HPLC purity: 91.1%).

### Example 143

### N-[(1-methyl-1H-imidazol-5-yl)methyl]-3-{[8-(1-methyl-1H-indol-6-yl)quinoxalin-6-yl]amino}pyridine-4-carboxamide

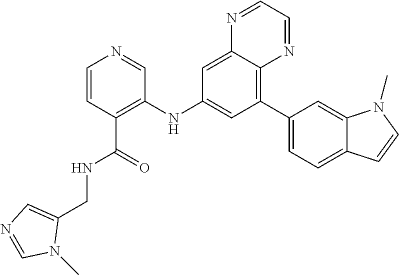 [SMILES: Cn1cncc1CNC(=O)c1ccncc1Nc1cc(-c2ccc3ccn(C)c3c2)c2nccnc2c1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 80 mg; 0.20 mmol; 1 eq.), (3-methyl-3H-imidazol-4-yl)-methylamine (29 mg; 0.25 mmol; 1.25 eq.), EDC.HCl (69 mg; 0.35 mmol; 1.80 eq.), HOBt hydrate (55 mg; 0.35 mmol; 1.80 eq.), triethylamine (0.13 mL; 0.98 mmol; 5 eq.) and dioxane (5 mL). Conditions: room temperature for 24 h. Purification by FCC (0% to 10% MeOH gradient in DCM) yields N-[(1-methyl-1H-imidazol-5-yl)methyl]-3-{[8-(1-methyl-1H-indol-6-yl)quinoxalin-6-yl]amino}pyridine-4-carboxamide (74 mg; 0.14 mmol; 73%; yellow powder; HPLC purity: 95.1%).

### Example 144

### 3-{[8-(1-Methyl-1H-indol-6-yl)quinoxalin-6-yl]amino}-N-[(pyridazin-3-yl)methyl]pyridine-4-carboxamide

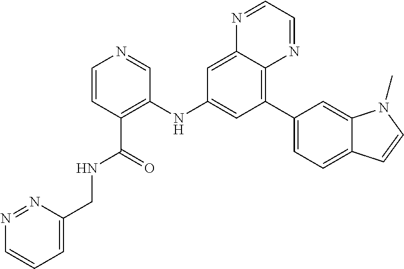 [SMILES: Cn1ccc2ccc(-c3cc(Nc4cnccc4C(=O)NCc4cccnn4)cc4nccnc34)cc21]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 80 mg; 0.20 mmol; 1 eq.), pyridazin-3-yl-methylamine (28 mg; 0.25 mmol; 1.25 eq.), EDC.HCl (69 mg; 0.35 mmol; 1.80 eq.), HOBt hydrate (55 mg; 0.35 mmol; 1.80 eq.), triethylamine (0.13 mL; 0.98 mmol; 5 eq.) and dioxane (5 mL). Conditions: room temperature for 24 h. Purification by FCC (0% to 10% MeOH gradient in DCM) yields 3-{[8-(1-methyl-1H-indol-6-yl)quinoxalin-6-yl]amino}-N-[(pyridazin-3-yl)methyl]pyridine-4-carboxamide (40 mg; 0.08 mmol; 41%; yellow powder; HPLC purity: 97%).

### Example 145

### 4-{[8-(1-Methyl-1H-indol-6-yl)quinoxalin-6-yl]amino}pyridine-3-carbonitrile

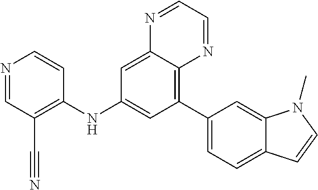 [SMILES: Cn1ccc2ccc(-c3cc(Nc4ccncc4C#N)cc4nccnc34)cc21]

A microwave vial is charged with 4-[(8-chloroquinoxalin-6-yl)amino]pyridine-3-carbonitrile (Example 146, 25 mg; 0.09 mmol; 1 eq.), 1-methyl-6-(4,4,5,5-tetramethyl-[1,3,2]dioxaborolan-2-yl)-1H-indole (30 mg; 0.10 mmol; 1.10 eq.), 2 M aqueous sodium carbonate (0.09 mL; 0.17 mmol; 2 eq.) and dioxane (1 mL). The reaction mixture is sparged with argon and tetrakis(triphenyl-phosphine)palladium(0) (5 mg; 4.3 μmol; 0.05 eq.) is added. The reaction mixture is stirred at 130° C. under microwave irradiation for 90 min. After coming back to room temperature, the reaction mixture is diluted with DCM and filtered through a pad of celite. The filtrate is washed with water and brine, dried over Na₂SO₄ and concentrated in vacuo. The crude product is purified by FCC (0% to 100% EtOAc gradient in hexane) to yield 4-{[8-(1-methyl-1H-indol-6-yl)quinoxalin-6-yl]amino}pyridine-3-carbonitrile (24 mg; 0.06 mmol; 68%; yellow powder; HPLC purity: 92.8%).

### Example 146

### 4-[(8-chloroquinoxalin-6-yl)amino]pyridine-3-carbonitrile

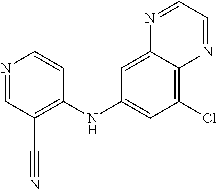 [SMILES: N#Cc1cnccc1Nc1cc(Cl)c2nccnc2c1]

The title compound is prepared according to General Procedure 1 described in Example 1, using 7-bromo-5-chloroquinoxaline (Intermediate 3, 100 mg; 0.40 mmol; 1 eq.), 4-aminopyridine-3-carbonitrile (65 mg; 0.52 mmol; 1.30 eq.), cesium carbonate (342 mg; 1.04 mmol; 2.60 eq.), BINAP (26 mg; 0.04 mmol; 0.10 eq.) and palladium(II) acetate (9 mg; 0.04 mmol; 0.10 eq.) in anhydrous dioxane (3 mL). Conditions: 3 hours at 120° C. Purification by FCC (0% to 100% EtOAc gradient in hexane followed by 0% to 10% MeOH gradient in EtOAc) yields 4-[(8-chloroquinoxalin-6-yl)amino]pyridine-3-carbonitrile (45 mg; 0.16 mmol; 39%; off-white powder; HPLC purity: 97.4%).

### Example 147

### N-(1-Acetylpiperidin-4-yl)-3-{[8-(1-methyl-1H-indol-6-yl)quinoxalin-6-yl]amino}pyridine-4-carboxamide

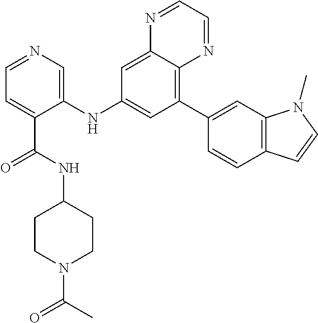 [SMILES: CC(=O)N1CCC(NC(=O)c2ccncc2Nc2cc(-c3ccc4ccn(C)c4c3)c3nccnc3c2)CC1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 50 mg; 0.13 mmol; 1 eq.), 1-(4-aminopiperidin-1-yl)-ethanone (22 μl; 0.16 mmol; 1.25 eq.), EDC.HCl (65 mg; 0.34 mmol; 2.70 eq.), HOBt hydrate (52 mg; 0.34 mmol; 2.70 eq.), triethylamine (0.08 mL; 0.63 mmol; 5 eq.) and dioxane (7 mL). Conditions: room temperature for 24 h. Purification by reversed-phase preparative HPLC (column: Gemini NX C18 5u 110A (100×30 mm), ACN gradient in water) affords N-(1-acetylpiperidin-4-yl)-3-[{3-(1-methyl-1H-indol-6-yl)quinoxalin-6-yl]amino}pyridine-4-carboxamide (20 mg; 0.04 mmol; 30%; yellow powder; HPLC purity: 100%).

### Example 148

### N-(1-Acetylpiperidin-3-yl)-3-{[8-(1-methyl-1H-indol-6-yl)quinoxalin-6-yl]amino}pyridine-4-carboxamide

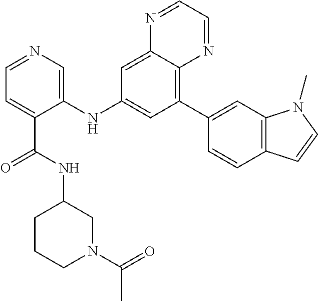 [SMILES: CC(=O)N1CCCC(NC(=O)c2ccncc2Nc2cc(-c3ccc4ccn(C)c4c3)c3nccnc3c2)C1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 50 mg; 0.13 mmol; 1 eq.), 1-(3-aminopiperidin-1-yl)-ethanone hydrochloride (28 mg; 0.16 mmol; 1.25 eq.), EDC.HCl (59 mg; 0.3 mmol; 2.40 eq.), HOBt hydrate (47 mg; 0.3 mmol; 2.40 eq.), triethylamine (0.08 mL; 0.63 mmol; 5 eq.) and dioxane (7 mL). Conditions: room temperature for 24 h. Purification by FCC (0% to 10% MeOH gradient in DCM) followed by preparative HPLC (column: Gemini NX C18 5u 110A (100×30 mm), ACN gradient in water) yields N-(1-acetylpiperidin-3-yl)-3-{[8-(1-methyl-1H-indol-6-yl)quinoxalin-6-yl]amino}pyridine-4-carboxamide formate salt (20 mg; 0.04 mmol; 30%; yellow powder; HPLC purity: 99.9%).

### Example 149

### 5-{[8-(1-methyl-1H-indol-6-yl)quinoxalin-6-yl]amino}pyrimidine-4-carboxamide

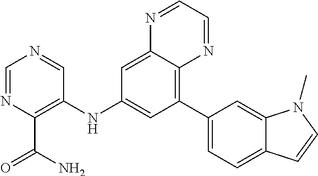 [SMILES: Cn1ccc2ccc(-c3cc(Nc4cncnc4C(N)=O)cc4nccnc34)cc21]

The title compound is prepared according to General Procedure 1 described in Example 1, using 5-bromopyrimidine-4-carbonitrile (40 mg; 0.22 mmol; 1 eq.), 7-chloro-5-(1-methyl-1H-indol-6-yl)quinoxaline (Intermediate 2B, 75 mg; 0.26 mmol; 1.20 eq.), cesium carbonate (170 mg; 0.52 mmol; 2.40 eq.), BINAP (21 mg; 0.03 mmol; 0.15 eq.) and palladium(II) acetate (8 mg; 0.03 mmol; 0.15 eq.) in anhydrous dioxane (2 mL). Conditions: 130° C. for 5 hours. Purification by FCC (0% to 100% EtOAc gradient in hexane) and preparative HPLC. The fractions containing the pure product are pooled and MeCN is evaporated. The resulting aqueous solution is basified with sodium bicarbonate and extracted with EtOAc. The organic phase is washed with brine, dried over sodium sulfate and evaporated to give 5-{[8-(1-methyl-1H-indol-6-yl)quinoxalin-6-yl]amino}pyrimidine-4-carboxamide (23 mg; 0.06 mmol; 27%; yellow powder; HPLC purity: 99.8%).

### Example 150

### 3-{[8-(3-Methyl-1-benzothiophen-5-yl)quinoxalin-6-yl]amino}pyridine-4-carbonitrile

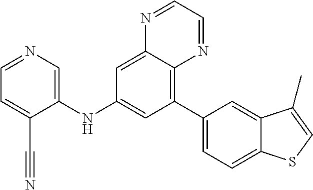 [SMILES: Cc1csc2ccc(-c3cc(Nc4cnccc4C#N)cc4nccnc34)cc12]

The title compound is prepared according to General Procedure 1 described in Example 1, using 7-chloro-5-(3-methyl-1-benzothiophen-5-yl)quinoxaline (Intermediate 66, 100 mg; 0.29 mmol; 1 eq.), 3-aminoisonicotinonitrile (45 mg; 0.37 mmol; 1.30 eq.), cesium carbonate (0.28 g; 0.86 mmol; 3 eq.), BINAP (18 mg; 0.03 mmol; 0.10 eq.) and palladium(II) acetate (7 mg; 0.03 mmol; 0.10 eq.) in dioxane (4 mL). Conditions: 1.5 h at 150° C. Purification by trituration in DCM affords 3-{[8-(3-methyl-1-benzothiophen-5-yl)quinoxalin-6-yl]amino}pyridine-4-carbonitrile (74 mg; 0.18 mmol; 64%; yellow powder; HPLC purity: 97.6%).

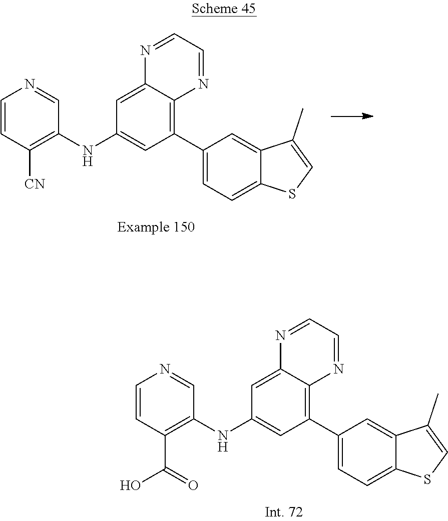 [SMILES: Cc1csc2ccc(-c3cc(Nc4cnccc4C#N)cc4nccnc34)cc12 ; Cc1csc2ccc(-c3cc(Nc4cnccc4C(=O)O)cc4nccnc34)cc12]

### Intermediate 72

### 3-{[8-(3-Methyl-1-benzothiophen-5-yl)quinoxalin-6-yl]amino}pyridine-4-carboxylic acid

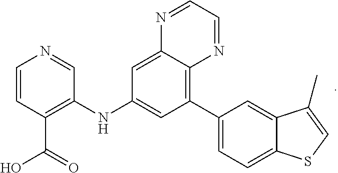 [SMILES: Cc1csc2ccc(-c3cc(Nc4cnccc4C(=O)O)cc4nccnc34)cc12]

A round-bottom flask is charged with water (2 mL) and KOH (209 mg; 3.72 mmol; 25 eq.) and the mixture is stirred until complete dissolution. Then 3-{[8-(3-methyl-1-benzothiophen-5-yl)quinoxalin-6-yl]amino}pyridine-4-carbonitrile (Example 150, 60 mg; 0.15 mmol; 1 eq.) and iPrOH (0.50 mL) are added, and the reaction mixture is stirred at 115° C. for 2 h. After coming back to room temperature, it is diluted with n-BuOH, neutralized with 1 M HCl and extracted with n-BuOH. The combined organic layers are dried over Na₂SO₄, filtered and concentrated in vacuo to afford crude 3-{[8-(3-methyl-1-benzothiophen-5-yl)quinoxalin-6-yl]amino}pyridine-4-carboxylic acid (90 mg; 0.21 mmol; >100%; yellow powder; UPLC purity: 98%) which is used in the next step without further purification.

### Example 151

### 3-{[8-(3-Methyl-1-benzothiophen-5-yl)quinoxalin-6-yl]amino}-N-(1-methylpyrrolidin-3-yl)pyridine-4-carboxamide

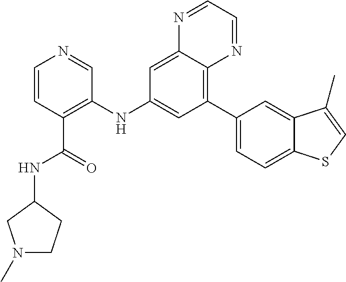 [SMILES: Cc1csc2ccc(-c3cc(Nc4cnccc4C(=O)NC4CCN(C)C4)cc4nccnc34)cc12]

The title compound is prepared according to General Procedure 13 described in Example 52, using 3-{[8-(3-methyl-1-benzothiophen-5-yl)quinoxalin-6-yl]amino}pyridine-4-carboxylic acid (Intermediate 72, 30 mg; 0.07 mmol; 1 eq.), 1-methylpyrrolidin-3-ylamine dihydrochloride (16 mg; 0.09 mmol; 1.25 eq.), EDC.HCl (25 mg; 0.13 mmol; 1.80 eq.), HOBt hydrate (20 mg; 0.13 mmol; 1.80 eq.), triethylamine (0.05 mL; 0.36 mmol; 5 eq.) in dioxane (5 mL). Conditions: room temperature for 24 h. Purification by FCC (0% to 15% MeOH gradient in DCM) affords 3-{[8-(3-methyl-1-benzothiophen-5-yl)quinoxalin-6-yl]amino}-N-(1-methylpyrrolidin-3-yl)pyridine-4-carboxamide (30 mg; 0.06 mmol; 81%; yellow powder; HPLC purity: 96.2%).

### Example 152

### N-(4-Methanesulfonylpyridin-3-yl)-8-(4-methoxyphenyl)quinoxalin-6-amine

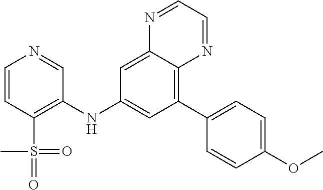

The title compound is prepared according to General Procedure 17 described in Example 66, using chloro-N-(4-methanesulfonylpyridin-3-yl)quinoxalin-6-amine (Intermediate 3B, 75 mg; 0.22 mmol; 1 eq.), sodium carbonate (115 mg; 1.09 mmol; 5 eq.), (4-methoxyphenyl)boronic acid (36 mg; 0.24 mmol; 1.10 eq.), tetrakis(triphenylphosphine)palladium(0) (13 mg; 0.01 mmol; 0.05 eq.) in water (0.50 mL), ethanol (0.50 mL) and toluene (1 mL). Conditions: 110° C. overnight. Purification by FCC (50% to 75% EtOAc gradient in hexane) yields N-(4-methanesulfonylpyridin-3-yl)-8-(4-methoxyphenyl)quinoxalin-6-amine (41 mg; 0.10 mmol; 46%; yellow powder; HPLC purity: 98.9%).

### Example 153

### N-(4-Methanesulfonylpyridin-3-yl)-8-(5-methoxy-2-methylphenyl)quinoxalin-6-amine

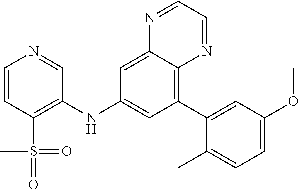

The title compound is prepared according to General Procedure 17 described in Example 66, using chloro-N-(4-methanesulfonylpyridin-3-yl)quinoxalin-6-amine (Intermediate 3B, 75 mg; 0.22 mmol; 1 eq.), sodium carbonate (115 mg; 1.09 mmol; 5 eq.), (5-methoxy-2-methyl-phenyl)boronic acid (40 mg; 0.24 mmol; 1.10 eq.), tetrakis(triphenylphosphine)palladium(0) (13 mg; 0.01 mmol; 0.05 eq.) in water (0.50 mL), ethanol (0.50 mL) and toluene (1 mL). Conditions: 110° C. overnight. Purification by FCC (50% to 75% EtOAc gradient in hexane) yields N-(4-methanesulfonylpyridin-3-yl)-8-(5-methoxy-2-methylphenyl)quinoxalin-6-amine (26 mg; 0.06 mmol; 28%; yellow powder; HPLC purity: 98.6%).

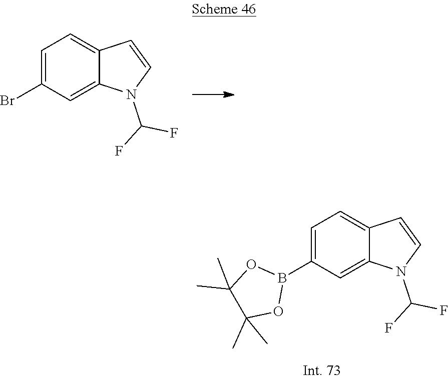 [SMILES: FC(F)n1ccc2ccc(Br)cc21 ; CC1(C)OB(c2ccc3ccn(C(F)F)c3c2)OC1(C)C]

### Intermediate 73

### 1-(Difluoromethyl)-6-(tetramethyl-1,3,2-dioxaborolan-2-yl)-1H-indole

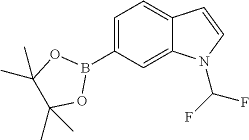 [SMILES: CC1(C)OB(c2ccc3ccn(C(F)F)c3c2)OC1(C)C]

The title compound is prepared according to General Procedure 8 described for Intermediate 14, using 6-bromo-1-difluoromethyl-1H-indole (670 mg; 1.58 mmol; 1 eq.), bis(pinacolato)diboron (520 mg; 2.05 mmol; 1.30 eq.), potassium acetate (309 mg; 3.15 mmol; 2 eq.) and Pd(dppf)Cl₂ (115 mg; 0.16 mmol; 0.10 eq.), dioxane (7 mL). Conditions: 100° C. overnight. Purification by FCC (0% to 20% EtOAc gradient in hexane) yields 1-(difluoromethyl)-6-(tetramethyl-1,3,2-dioxaborolan-2-yl)-1H-indole (392 mg; 1.18 mmol; 75%; white waxy solid; UPLC purity: 91%).

### Example 154

### 8-[1-(Difluoromethyl)-1H-indol-6-yl]-N-(4-methanesulfonylpyridin-3-yl)quinoxalin-6-amine

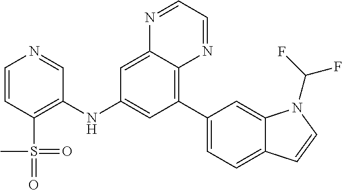 [SMILES: CS(=O)(=O)c1ccncc1Nc1cc(-c2ccc3ccn(C(F)F)c3c2)c2nccnc2c1]

The title compound is prepared according to General Procedure 17 described in Example 66, using chloro-N-(4-methanesulfonylpyridin-3-yl)quinoxalin-6-amine (Intermediate 3B, 75 mg; 0.22 mmol; 1 eq.), sodium carbonate (115 mg; 1.09 mmol; 5 eq.), 1-(difluoromethyl)-6-(tetramethyl-1,3,2-dioxaborolan-2-yl)-1H-indole (Intermediate 73, 77 mg; 0.24 mmol; 1.10 eq.), tetrakis(triphenylphosphine)palladium(0) (13 mg; 0.01 mmol; 0.05 eq.) in water (0.50 mL), ethanol (0.50 mL) and toluene (1 mL). Conditions: 110° C. overnight. Purification by FCC (100% EtOAc) yields N-(4-methanesulfonylpyridin-3-yl)-8-(5-methoxy-2-methylphenyl)quinoxalin-6-amine (26 mg; 0.06 mmol; 25%; yellow powder; HPLC purity: 99.1%).

### Example 157, General Procedure 23

### 8-(4-Bromophenyl)-N-(4-methanesulfonylpyridin-3-yl)quinoxalin-6-amine

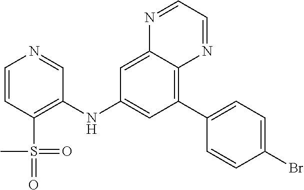 [SMILES: CS(=O)(=O)c1ccncc1Nc1cc(-c2ccc(Br)cc2)c2nccnc2c1]

Anhydrous copper(II) bromide (240 mg; 1.07 mmol; 1.20 eq.), tert-butylnitrite (160 μl; 1.34 mmol; 1.50 eq.) and degassed anhydrous acetonitrile (5 mL) are placed in a 10-mL round bottom flask under argon. The resulting mixture is cooled to 0° C. with rapid stirring. 8-(4-Aminophenyl)-N-(4-methanesulfonylpyridin-3-yl)quinoxalin-6-amine (Example 131, 350 mg; 0.89 mmol; 1 eq.) is slowly added as a solution in degassed anhydrous DMF (5 mL) over a period of 5 min. The reaction is allowed to come back to room temperature and kept with stirring under Ar for 2 hours. The mixture is poured onto ice/water (50 mL) and extracted with DCM. The organic phase is washed with brine, dried (sodium sulfate) and evaporated. The crude is purified by FCC (70% acetone in hexane) and reversed-phase preparative HPLC (column: Gemini NX C18 5u 110A (100×30 mm), ACN gradient in water). The pure fractions are pooled, MeCN is evaporated and the resulting solution is basified with NaHCO₃. It is then extracted with DCM (twice) and the combined organic layers are dried (sodium sulfate) and evaporated to dryness to yield 8-(4-bromophenyl)-N-(4-methanesulfonylpyridin-3-yl)quinoxalin-6-amine (60 mg; 0.13 mmol; 15%; pale yellow powder; HPLC purity: 98.6%).

### Example 158

### 8-(3-Bromophenyl)-N-(4-methanesulfonylpyridin-3-yl)quinoxalin-6-amine

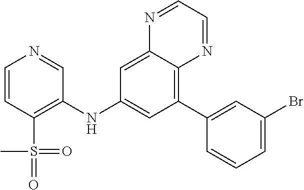 [SMILES: CS(=O)(=O)c1ccncc1Nc1cc(-c2cccc(Br)c2)c2nccnc2c1]

The title compound is prepared according to General Procedure 23 described in Example 157, using anhydrous copper(II) bromide (240 mg; 1.07 mmol; 1.20 eq.), tert-butylnitrite (160 μl; 1.34 mmol; 1.50 eq.), 8-(3-aminophenyl)-N-(4-methanesulfonylpyridin-3-yl)quinoxalin-6-amine (350 mg; 0.89 mmol; 1 eq.) in degassed anhydrous acetonitrile (5 mL) and DMF (5 mL). Conditions: 0° C. to room temperature for 2 hours. Purification by FCC (30% acetone in hexane), and by reversed-phase preparative HPLC (column: Gemini NX C18 5u 110A (100×30 mm), ACN gradient in water). The pure fractions are pooled, MeCN is evaporated and the resulting solution is basified with NaHCO₃. It is then extracted with DCM (twice) and the combined organic layers are dried (sodium sulfate) and evaporated to dryness to yield 8-(3-bromophenyl)-N-(4-methanesulfonylpyridin-3-yl)quinoxalin-6-amine (97 mg; 0.21 mmol; 24%; yellow powder; HPLC purity: 100%).

### Example 160

### 2-Aminopyrimidin-4-yl3-{[8-(1-methyl-1H-indol-6-yl)quinoxalin-6-yl]amino}pyridine-4-carboxylate

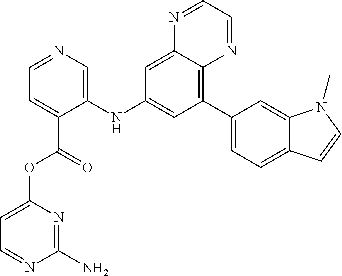 [SMILES: Cn1ccc2ccc(-c3cc(Nc4cnccc4C(=O)Oc4ccnc(N)n4)cc4nccnc34)cc21]

The title compound is prepared according to General Procedure 13 described in Example 52, using 3-{[8-(3-methyl-1-benzothiophen-5-yl)quinoxalin-6-yl]amino}pyridine-4-carboxylic acid (Intermediate 72, 45 mg; 0.11 mmol; 1 eq.), 2-amino-1H-pyrimidin-4-one (20 mg; 0.17 mmol; 1.50 eq.), EDC.HCl (39 mg; 0.2 mmol; 1.80 eq.), HOBt hydrate (31 mg; 0.2 mmol; 1.80 eq.), triethylamine (0.07 mL; 0.59 mmol; 5 eq.) in dioxane (5 mL) and DMF (2 mL). Conditions: 75° C. for 6 h. Purification by FCC (0% to 10% MeOH gradient in DCM) affords 2-aminopyrimidin-4-yl-3-{[8-(1-methyl-1H-indol-6-yl)quinoxalin-6-yl]amino}pyridine-4-carboxylate (36 mg; 0.07 mmol; 60%; yellow powder; HPLC purity: 91.3%).

### Example 161

### 8-(1,2-Benzothiazol-5-yl)-N-(4-methanesulfonylpyridin-3-yl)quinoxalin-6-amine

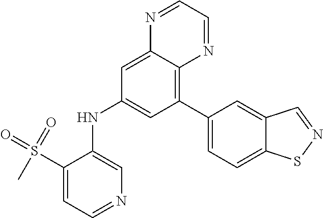 [SMILES: CS(=O)(=O)c1ccncc1Nc1cc(-c2ccc3sncc3c2)c2nccnc2c1]

The title compound is prepared according to General Procedure 17 described in Example 66, using chloro-N-(4-methanesulfonylpyridin-3-yl)quinoxalin-6-amine (Intermediate 3B, 60 mg; 0.17 mmol; 1 eq.), sodium carbonate (92 mg; 0.87 mmol; 5 eq.), 5-(4,4,5,5-tetramethyl-[1,3,2]dioxaborolan-2-yl)-benzo[d]isothiazole (69 mg; 0.19 mmol; 1.10 eq.), tetrakis(triphenyl-phosphine)palladium(0) (11 mg; 0.01 mmol; 0.05 eq.) in water (0.50 mL), ethanol (0.50 mL) and toluene (1 mL). Conditions: 110° C. overnight. Purification by FCC (100% EtOAc) affords 8-(1,2-benzothiazol-5-yl)-N-(4-methanesulfonylpyridin-3-yl)quinoxalin-6-amine (53 mg; 0.12 mmol; 69%; yellow powder; HPLC purity: 97.6%).

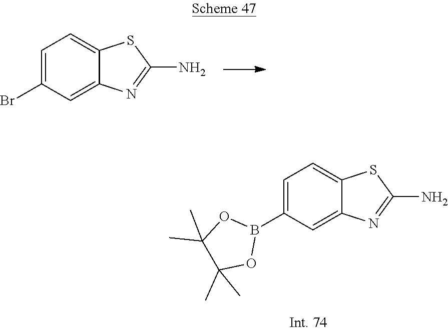 [SMILES: Nc1nc2cc(Br)ccc2s1 ; CC1(C)OB(c2ccc3sc(N)nc3c2)OC1(C)C]

### Intermediate 74

### 5-(Tetramethyl-1,3,2-dioxaborolan-2-yl)-1,3-benzothiazol-2-amine

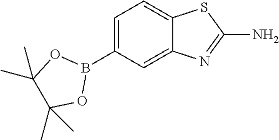 [SMILES: CC1(C)OB(c2ccc3sc(N)nc3c2)OC1(C)C]

The title compound is prepared according to General Procedure 8 described for Intermediate 14, using 5-bromobenzothiazol-2-ylamine (1.70 g; 7.42 mmol; 1 eq.), bis(pinacolato)diboron (2.83 g; 11.13 mmol; 1.50 eq.), potassium acetate (1.60 g; 16.32 mmol; 2.20 eq.) and Pd(dppf)Cl₂ (618 mg; 0.817 mmol; 0.10 eq.), in dioxane (7 mL). Conditions: 100° C. overnight. Purification by FCC (0% to 30% EtOAc gradient in hexane) yields 5-(tetramethyl-1,3,2-dioxaborolan-2-yl)-1,3-benzothiazol-2-amine (1.16 g; 3.27 mmol; 44%; white waxy solid; UPLC purity: 78%).

### Example 162

### 8-(2-Amino-1,3-benzothiazol-5-yl)-N-(4-methanesulfonylpyridin-3-yl)quinoxalin-6-amine

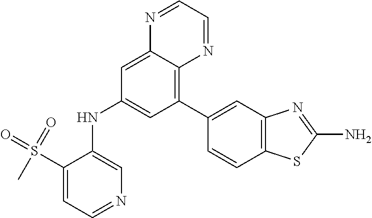 [SMILES: CS(=O)(=O)c1ccncc1Nc1cc(-c2ccc3sc(N)nc3c2)c2nccnc2c1]

The title compound is prepared according to General Procedure 17 described in Example 66, using chloro-N-(4-methanesulfonylpyridin-3-yl)quinoxalin-6-amine (Intermediate 3B, 120 mg; 0.35 mmol; 1 eq.), sodium carbonate (184 mg; 1.74 mmol; 5 eq.), 5-(tetramethyl-1,3,2-dioxaborolan-2-yl)-1,3-benzothiazol-2-amine (Intermediaire 74, 264 mg; 0.38 mmol; 1.10 eq.), tetrakis(triphenylphosphine)palladium(0) (21 mg; 0.02 mmol; 0.05 eq.) in water (0.50 mL), ethanol (0.50 mL) and toluene (1 mL). THF was used for extractions. Conditions: 110° C. overnight. Purification by FCC (50-70% acetone gradient in hexane) affords 8-(2-amino-1,3-benzothiazol-5-yl)-N-(4-methanesulfonylpyridin-3-yl)quinoxalin-6-amine (96 mg; 0.21 mmol; 61%; yellow powder; HPLC purity: 99.6%).

### Example 163

### N-(4-Methanesulfonylpyridin-3-yl)-8-[3-(trifluoromethoxy)phenyl]quinoxalin-6-amine

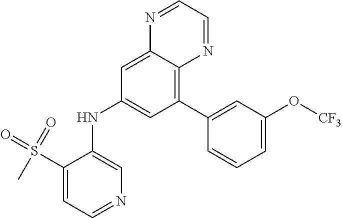 [SMILES: CS(=O)(=O)c1ccncc1Nc1cc(-c2cccc(OC(F)(F)F)c2)c2nccnc2c1]

The title compound is prepared according to General Procedure 17 described in Example 66, using chloro-N-(4-methanesulfonylpyridin-3-yl)quinoxalin-6-amine (Intermediate 3B, 100 mg; 0.29 mmol; 1 eq.), sodium carbonate (154 mg; 1.45 mmol; 5 eq.), 3-(trifluoromethoxy)phenyl]boronic acid (72 mg; 0.35 mmol; 1.20 eq.), tetrakis(triphenylphosphine)palladium(0) (18 mg; 0.01 mmol; 0.05 eq.) in water (0.50 mL), ethanol (0.50 mL) and toluene (1 mL). Conditions: 110° C. overnight. Purification by FCC (25% acetone in hexane) followed by reversed-phase preparative HPLC (column: Gemini NX C18 5u 110A (100×30 mm), ACN gradient in water). The pure fractions are pooled, MeCN is evaporated and the resulting solution is basified with NaHCO₃. It is then extracted with DCM (twice) and the combined organic layers are dried (sodium sulfate) and evaporated to dryness to yield N-(4-methanesulfonylpyridin-3-yl)-8-[3-(trifluoromethoxy)phenyl]quinoxalin-6-amine (112 mg; 0.24 mmol; 84%; yellow powder; HPLC purity: 99.9%).

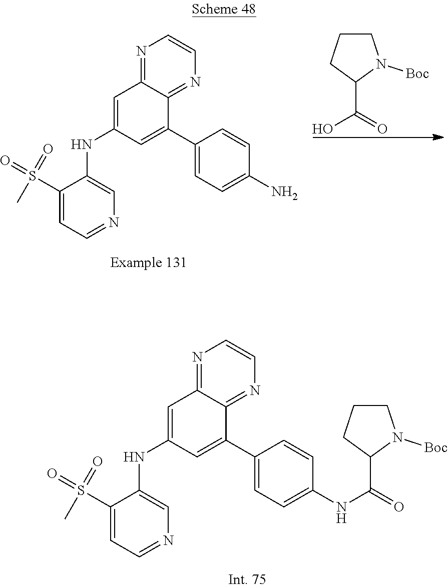 [SMILES: CS(=O)(=O)c1ccncc1Nc1cc(-c2ccc(N)cc2)c2nccnc2c1 ; CC(C)(C)OC(=O)N1CCCC1C(=O)O ; CC(C)(C)OC(=O)N1CCCC1C(=O)Nc1ccc(-c2cc(Nc3cnccc3S(C)(=O)=O)cc3nccnc23)cc1]

### Intermediate 75

### tert-Butyl 2-[(4-{7-[(4-methanesulfonylpyridin-3-yl)amino]quinoxalin-5-yl}phenyl)carbamoyl]pyrrolidine-1-carboxylate

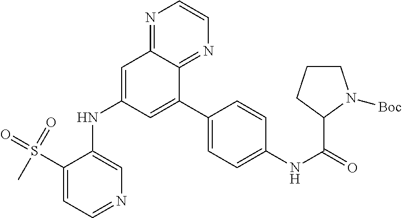 [SMILES: CC(C)(C)OC(=O)N1CCCC1C(=O)Nc1ccc(-c2cc(Nc3cnccc3S(C)(=O)=O)cc3nccnc23)cc1]

The title compound is prepared according to General Procedure 22 described in Example 126, using 8-(4-aminophenyl)-N-(4-methanesulfonylpyridin-3-yl)quinoxalin-6-amine (Example 131, 250 mg; 0.64 mmol; 1 eq.), Boc-DL-Pro-OH (192 mg; 0.89 mmol; 1.40 eq.), HATU (316 mg; 0.83 mmol; 1.30 eq.) and DIPEA (0.22 mL; 1.28 mmol; 2 eq.) in anhydrous DMF (10 mL). Conditions: overnight at room temperature. Purification by FCC (50% acetone in hexane) affords tert-butyl 2-[(4-{7-[(4-methanesulfonylpyridin-3-yl)amino]quinoxalin-5-yl}phenyl)carbamoyl]pyrrolidine-1-carboxylate (226 mg; 0.38 mmol; 58%; beige powder; UPLC purity: 97%).

### Example 164

### N-(4-{7-[(4-methanesulfonylpyridin-3-yl)amino]quinoxalin-5-yl}phenyl)pyrrolidine-2-carboxamide

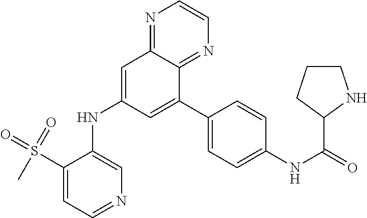 [SMILES: CS(=O)(=O)c1ccncc1Nc1cc(-c2ccc(NC(=O)C3CCCN3)cc2)c2nccnc2c1]

tert-Butyl 2-[(4-{7-[(4-methanesulfonylpyridin-3-yl)amino]quinoxalin-5-yl}phenyl)carbamoyl]pyrrolidine-1-carboxylate (Intermediate 75, 226 mg; 0.38 mmol; 1 eq.) is dissolved in DCM (5 mL) and trifluoroacetic acid (3 mL) is added. The mixture is left with stirring at room temperature for 1 hour. The solvents are evaporated, and the residue is coevaporated twice with toluene, dissolved in a minimum amount of DCM and the solution is added dropwise to rapidly stirring saturated aq. NaHCO₃. The phases are separated and the aqueous phase is extracted twice with DCM. The combined organic layers are dried (sodium sulfate), filtered and evaporated to a residue which is purified by FCC (2 to 15% MeOH gradient in DCM) to afford N-(4-{7-[(4-methanesulfonylpyridin-3-yl)amino]quinoxalin-5-yl}phenyl)pyrrolidine-2-carboxamide (104 mg; 0.21 mmol; 55%; yellow powder; HPLC purity: 98.6%).

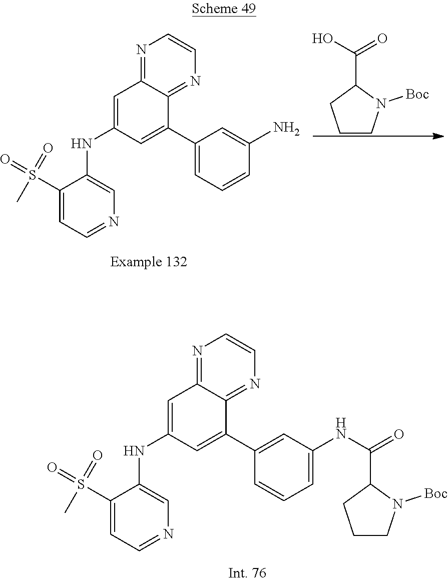 [SMILES: CS(=O)(=O)c1ccncc1Nc1cc(-c2cccc(N)c2)c2nccnc2c1 ; CC(C)(C)OC(=O)N1CCCC1C(=O)O ; CC(C)(C)OC(=O)N1CCCC1C(=O)Nc1cccc(-c2cc(Nc3cnccc3S(C)(=O)=O)cc3nccnc23)c1]

### Intermediate 76

### tert-Butyl 2-[(3-{7-[(4-methanesulfonylpyridin-3-yl)amino]quinoxalin-5-yl}phenyl)carbamoyl]pyrrolidine-1-carboxylate

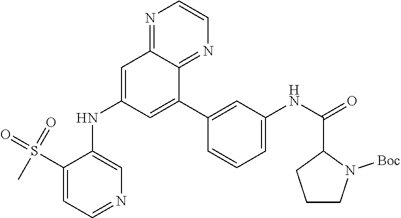 [SMILES: CC(C)(C)OC(=O)N1CCCC1C(=O)Nc1cccc(-c2cc(Nc3cnccc3S(C)(=O)=O)cc3nccnc23)c1]

The title compound is prepared according to General Procedure 22 described in Example 126, using 8-(3-aminophenyl)-N-(4-methanesulfonylpyridin-3-yl)quinoxalin-6-amine (Example 132, 127 mg; 0.32 mmol; 1 eq.), Boc-DL-Pro-OH (98 mg; 0.45 mmol; 1.40 eq.) and HATU (160 mg; 0.42 mmol; 1.30 eq.) and DIPEA (0.11 mL; 0.65 mmol; 2 eq.) in anhydrous DMF (6 mL). Conditions: overnight at room temperature. Purification by FCC (50% acetone in hexane) affords tert-butyl 2-[(3-{7-[(4-methanesulfonylpyridin-3-yl)amino]quinoxalin-5-yl}phenyl)carbamoyl]pyrrolidine-1-carboxylate (116 mg; 0.20 mmol; 55%; beige powder; UPLC purity: 90%).

### Example 165

### N-(3-{7-[(4-Methanesulfonylpyridin-3-yl)amino]quinoxalin-5-yl}phenyl)pyrrolidine-2-carboxamide

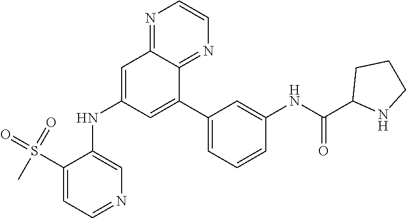 [SMILES: CS(=O)(=O)c1ccncc1Nc1cc(-c2cccc(NC(=O)C3CCCN3)c2)c2nccnc2c1]

tert-Butyl 2-[(3-{7-[(4-methanesulfonylpyridin-3-yl)amino]quinoxalin-5-yl}phenyl)carbamoyl]pyrrolidine-1-carboxylate (Intermediate 76, 116 mg; 0.20 mmol; 1 eq.) is dissolved in DCM (5 mL) and trifluoroacetic acid (3 mL) is added. The mixture is left with stirring at room temperature for 1 hour. The solvents are evaporated, and the residue is coevaporated twice with toluene, dissolved in a minimum amount of DCM and the solution is added dropwise to rapidly stirring saturated aq. NaHCO₃. The phases are separated and the aqueous phase is extracted twice with DCM. The combined organic layers are dried (sodium sulfate), filtered and evaporated to a residue which is purified by FCC (2 to 15% MeOH gradient in DCM) to afford N-(3-{7-[(4-methanesulfonylpyridin-3-yl)amino]quinoxalin-5-yl}phenyl)pyrrolidine-2-carboxamide (35 mg; 0.07 mmol; 35%; yellow powder; HPLC purity: 97.6%).

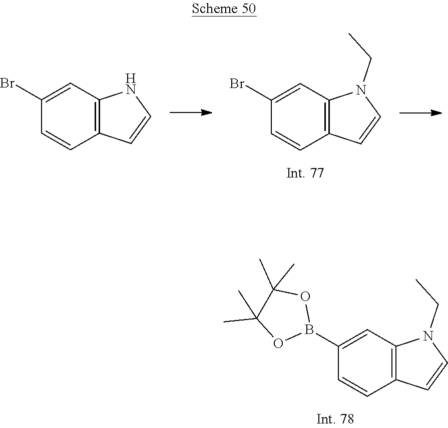 [SMILES: Brc1ccc2cc[nH]c2c1 ; CCn1ccc2ccc(Br)cc21 ; CCn1ccc2ccc(B3OC(C)(C)C(C)(C)O3)cc21]

### Intermediate 77

### 6-Bromo-1-ethyl-1H-indole

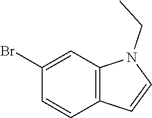

The title compound is prepared according to General Procedure 14 described for Intermediate 45, using 6-bromo-1H-indole (0.50 g; 2.55 mmol; 1 eq.), NaH (60% in mineral oil, 0.20 g; 5.10 mmol; 2 eq.), iodoethane (0.27 mL; 3.32 mmol; 1.30 eq.) in dry THF (10 mL). Conditions: 0° C. to room temperature for 2 h. The crude 6-bromo-1-ethyl-1H-indole (594 mg; 2.35 mmol; 92%; brown oil; UPLC purity: 89%) is used in the next step without further purification.

### Intermediate 78

### 1-Ethyl-6-(tetramethyl-1,3,2-dioxaborolan-2-yl)-1H-indole

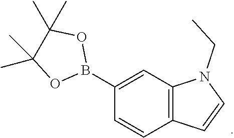 [SMILES: CCn1ccc2ccc(B3OC(C)(C)C(C)(C)O3)cc21]

The title compound is prepared according to General Procedure 8 described for Intermediate 14, using 6-bromo-1-ethyl-1H-indole (Intermediate 76, 226 mg; 0.99 mmol; 1 eq.), bis(pinacolato)diboron (326 mg; 1.28 mmol; 1.30 eq.), potassium acetate (194 mg; 1.98 mmol; 2 eq.), Pd(dppf)Cl₂ (7 mg; 0.01 mmol; 0.01 eq.) in dioxane (3 mL). Conditions: 100° C. overnight. Purification by FCC (5% EtOAc in hexane) affords 1-ethyl-6-(tetramethyl-1,3,2-dioxaborolan-2-yl)-1H-indole (195 mg; 0.58 mmol; 59%; white powder; UPLC purity: 81%).

### Example 166

### 8-(1-Ethyl-1H-indol-6-yl)-N-(4-methanesulfonylpyridin-3-yl)quinoxalin-6-amine

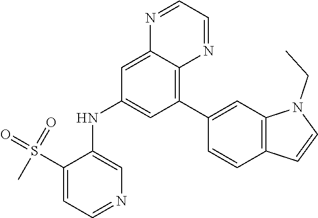 [SMILES: CCn1ccc2ccc(-c3cc(Nc4cnccc4S(C)(=O)=O)cc4nccnc34)cc21]

The title compound is prepared according to General Procedure 17 described in Example 66, using sodium carbonate (302 mg; 2.85 mmol; 5 eq.), 1-1-ethyl-6-(tetramethyl-1,3,2-dioxaborolan-2-yl)-1H-indole (Intermediate 78, 186 mg; 0.68 mmol; 1.20 eq.), chloro-N-(4-methanesulfonylpyridin-3-yl)quinoxalin-6-amine (Intermediate 3B, 191 mg; 0.57 mmol; 1 eq.) and tetrakis(triphenylphosphine)palladium(0) (35 mg; 0.03 mmol; 0.05 eq.) in toluene (3 mL), ethanol (1.50 mL) and water (1.50 mL). Conditions: 110° C. overnight. Purification by FCC (80% EtOAc) affords 8-(1-ethyl-1H-indol-6-yl)-N-(4-methanesulfonylpyridin-3-yl)quinoxalin-6-amine (175 mg; 0.37 mmol; 64%; yellow powder; HPLC purity: 92.7%).

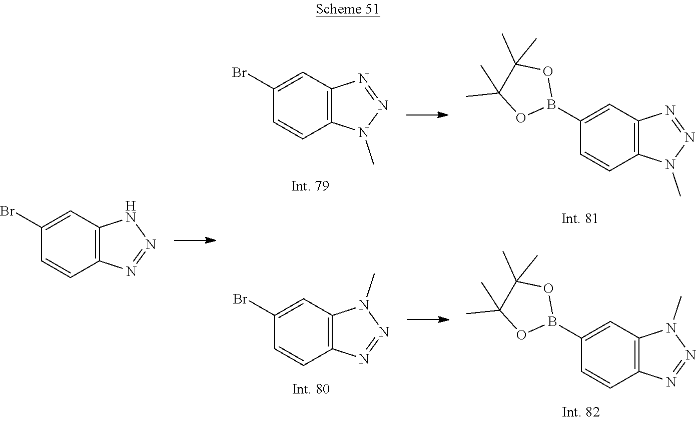 [SMILES: Cn1nnc2cc(Br)ccc21 ; Cn1nnc2cc(B3OC(C)(C)C(C)(C)O3)ccc21 ; Brc1ccc2nn[nH]c2c1 ; Cn1nnc2ccc(Br)cc21 ; Cn1nnc2ccc(B3OC(C)(C)C(C)(C)O3)cc21]

### Intermediate 79 and Intermediate 80

### 5-Bromo-1-methyl-1H-1,2,3-benzotriazole (79)

### 6-Bromo-1-methyl-1H-1,2,3-benzotriazole (80)

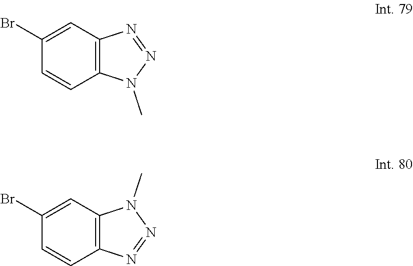

A flask equipped with a stirring bar and flushed with Ar is loaded with 5-bromo-1H-benzotriazole (200 mg; 1.01 mmol; 1 eq.), potassium iodide (17 mg; 0.10 mmol; 0.10 eq.), potassium carbonate (698 mg; 5.05 mmol; 5 eq.) and acetone (20 mL). The mixture is cooled in an ice bath and iodomethane (0.08 mL; 1.11 mmol; 1.10 eq.) is added via syringe. After 15 minutes, the bath is removed and the mixture is stirred for 48 h at room temperature. The reaction mixture is then filtered, and the filtrate is evaporated in vacuo. The residue is purified by FCC (0-30% EtOAc gradient in hexane) to provide two regioisomers:

5-bromo-1-methyl-1H-1,2,3-benzotriazole (79, 63 mg, 0.30 mmol, 29%; UPLC purity: 100%).

6-bromo-1-methyl-1H-1,2,3-benzotriazole (80, 57 mg, 0.27 mmol, 27%; UPLC purity: 100%).

### Intermediate 81

### 1-Methyl-5-(tetramethyl-1,3,2-dioxaborolan-2-yl)-1H-1,2,3-benzotriazole

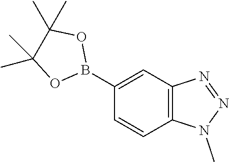 [SMILES: Cn1nnc2cc(B3OC(C)(C)C(C)(C)O3)ccc21]

The title compound is prepared according to General Procedure 8 described for Intermediate 14, using 5-bromo-1-methyl-1H-1,2,3-benzotriazole (Intermediate 79, 50 mg; 0.24 mmol; 1 eq.), bis(pinacolato)diboron (120 mg; 0.47 mmol; 2 eq.), potassium acetate (139 mg; 1.41 mmol; 6 eq.), Pd(dppf)Cl₂ (30 mg; 0.04 mmol; 0.15 eq.) in dioxane (3 mL). Conditions: 90° C. overnight. The crude 1-methyl-5-(tetramethyl-1,3,2-dioxaborolan-2-yl)-1H-1,2,3-benzotriazole (80 mg; 0.14 mmol; 61%; brown solid; UPLC purity: 65%) is used in the next step without further purification.

### Intermediate 82

### 1-Methyl-6-(tetramethyl-1,3,2-dioxaborolan-2-yl)-1H-1,2,3-benzotriazole

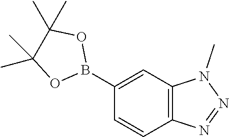 [SMILES: Cn1nnc2ccc(B3OC(C)(C)C(C)(C)O3)cc21]

The title compound is prepared according to General Procedure 8 described for Intermediate 14, using 6-bromo-1-methyl-1H-1,2,3-benzotriazole (Intermediate 80, 45 mg; 0.21 mmol; 1 eq.), bis(pinacolato)diboron (108 mg; 0.42 mmol; 2 eq.), potassium acetate (125 mg; 1.27 mmol; 6 eq.), Pd(dppf)Cl₂ (27 mg; 0.03 mmol; 0.15 eq.) in DMSO (2 mL). Conditions: 90° C. overnight. The crude 1-methyl-6-(tetramethyl-1,3,2-dioxaborolan-2-yl)-1H-1,2,3-benzotriazole (80 mg; 0.10 mmol; 45%; brown solid; UPLC purity: 85%) is used in the next step without further purification.

### Example 167

### N-(4-Methanesulfonylpyridin-3-yl)-8-(1-methyl-1H-1,2,3-benzotriazol-5-yl)quinoxalin-6-amine

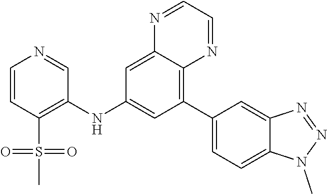 [SMILES: Cn1nnc2cc(-c3cc(Nc4cnccc4S(C)(=O)=O)cc4nccnc34)ccc21]

The title compound is prepared according to General Procedure 17 described in Example 66, using sodium carbonate (61 mg; 0.58 mmol; 5 eq.), 1-methyl-5-(tetramethyl-1,3,2-dioxaborolan-2-yl)-1H-1,2,3-benzotriazole (Intermediate 81, 71 mg; 0.13 mmol; 1.10 eq.), chloro-N-(4-methanesulfonylpyridin-3-yl)quinoxalin-6-amine (Intermediate 3B, 40 mg; 0.12 mmol; 1 eq.) and tetrakis(triphenylphosphine)palladium(0) (7 mg; 0.01 mmol; 0.05 eq.) in toluene (2 mL), ethanol (1 mL) and water (1 mL). Conditions: 100° C. overnight. Purification by reversed-phase preparative HPLC (column: Gemini NX C18 5u 110A (100×30 mm), ACN gradient in water). The pure fractions are pooled, MeCN is evaporated and the resulting solution is basified with aq. 2M NaOH. It is then extracted with DCM (twice) and the combined organic layers are dried (sodium sulfate) and evaporated to dryness to yield affords N-(4-methanesulfonylpyridin-3-yl)-8-(1-methyl-1H-1,2,3-benzotriazol-5-yl)quinoxalin-6-amine (11 mg; 0.02 mmol; 21%; yellow powder; HPLC purity:

99.5%).

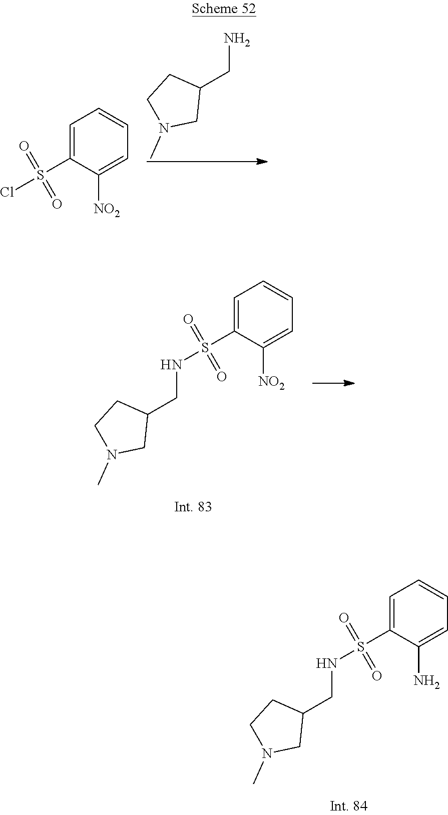

### Intermediate 83, General Procedure 24

### N-[(1-methylpyrrolidin-3-yl)methyl]-2-nitrobenzene-1-sulfonamide

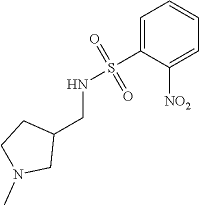 [SMILES: CN1CCC(CNS(=O)(=O)c2ccccc2[N+](=O)[O-])C1]

Triethylamine (175 μL; 1.35 mmol; 3 eq.) and 2-nitrobenzenesulfonyl chloride (100 mg; 0.45 mmol; 1 eq.) are added to a solution of 1-methylpyrrolidin-3-yl-methylamine (59 μL; 0.54 mmol; 1.20 eq.) in DCM (4 mL). The reaction mixture is stirred at room temperature for two days. It is then evaporated under reduced pressure and the residue is partitioned between water and a mixture of DCM:isopropanol (4:1). The aqueous layer is extracted with DCM:isopropanol (4:1) and the combined organic layers are dried over Na₂SO₄, filtered and concentrated. The residue is purified by FCC (silica deactivated with ammonia, 0% to 10% MeOH gradient in DCM) to afford N-[(1-methylpyrrolidin-3-yl)methyl]-2-nitrobenzene-1-sulfonamide (110 mg; 0.36 mmol; 80%; colorless gel; UPLC purity: 80%).

### Intermediate 84, General Procedure 25

### 2-amino-N-[(1-methylpyrrolidin-3-yl)methyl]benzene-1-sulfonamide

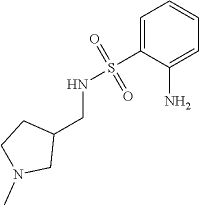 [SMILES: CN1CCC(CNS(=O)(=O)c2ccccc2N)C1]

Iron powder (80 mg; 1.42 mmol; 4 eq.) is added to a solution of N-[(1-methylpyrrolidin-3-yl)methyl]-2-nitrobenzene-1-sulfonamide (Intermediate 83, 109 mg; 0.36 mmol; 1 eq.) in acetic acid (3 mL) and the resulting mixture is stirred at 85° C. for 2 hours. The reaction mixture filtered through celite, evaporated under reduced pressure and the residue is partitioned between aq. 1M NaOH and a mixture of DCM:isopropanol (4:1). The aqueous layer is extracted with DCM:isopropanol (4:1) and the combined organic layers are dried over Na₂SO₄, filtered and concentrated to afford crude 2-amino-N-[(1-methylpyrrolidin-3-yl)methyl]benzene-1-sulfonamide (37 mg; 0.09 mmol; 24%; white semi-solid; UPLC purity: 62%) which is used in the next step without further purification.

### Example 168

### 2-{[8-(1-Methyl-1H-indol-6-yl)quinoxalin-6-yl]amino}-N-[(1-methylpyrrolidin-3-yl)methyl]benzene-1-sulfonamide

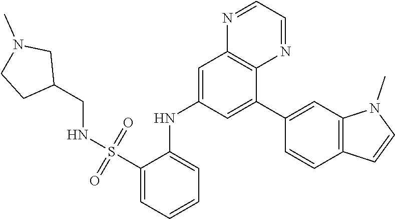 [SMILES: CN1CCC(CNS(=O)(=O)c2ccccc2Nc2cc(-c3ccc4ccn(C)c4c3)c3nccnc3c2)C1]

The title compound is prepared according to General Procedure 1 described in Example 1, using 7-7-chloro-5-(1-methyl-1H-indol-6-yl)quinoxaline (Intermediate 2B, 19 mg; 0.06 mmol; 0.75 eq.), cesium carbonate (139 mg; 0.43 mmol; 5 eq.), 2-amino-N-[(1-methylpyrrolidin-3-yl)methyl]benzene-1-sulfonamide (Intermediate 84, 37 mg; 0.09 mmol; 1 eq.), palladium(II) acetate (2 mg; 0.01 mmol; 0.10 eq.) and BINAP (5 mg; 0.01 mmol; 0.10 eq.) in dioxane (20 mL). Conditions: 1.5 h at 150° C. Purification by FCC (silica deactivated with ammonia, 0% to 10% MeOH gradient in DCM) affords 2-{[8-(1-methyl-1H-indol-6-yl)quinoxalin-6-yl]amino}-N-[(1-methylpyrrolidin-3-yl)methyl]benzene-1-sulfonamide (18 mg; 0.03 mmol; 40%; yellow powder; HPLC purity: 98.4%).

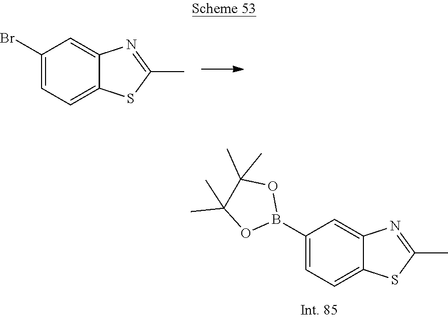

### Intermediate 85

### 2-Methyl-5-(tetramethyl-1,3,2-dioxaborolan-2-yl)-1,3-benzothiazole

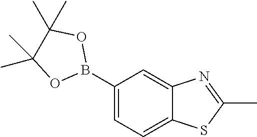

The title compound is prepared according to General Procedure 8 described for Intermediate 14, using 5-bromo-2-methylbenzothiazole (200 mg; 0.84 mmol; 1 eq.), bis(pinacolato)diboron (427 mg; 1.68 mmol; 2 eq.), potassium acetate (496 mg; 5.05 mmol; 6 eq.) and Pd(dppf)Cl₂ (105 mg; 0.13 mmol; 0.15 eq.) in DMSO (2 mL). The crude 2-methyl-5-(tetramethyl-1,3,2-dioxaborolan-2-yl)-1,3-benzothiazole (260 mg; 0.66 mmol; 78%; brown solid; UPLC purity: 87%).

### Example 169

### N-(4-methanesulfonylpyridin-3-yl)-8-(2-methyl-1,3-benzothiazol-5-yl)quinoxalin-6-amine

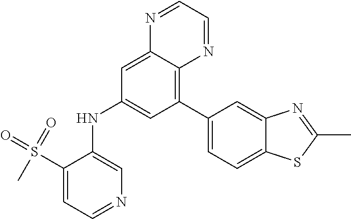 [SMILES: Cc1nc2cc(-c3cc(Nc4cnccc4S(C)(=O)=O)cc4nccnc34)ccc2s1]

The title compound is prepared according to General Procedure 17 described in Example 66, using chloro-N-(4-methanesulfonylpyridin-3-yl)quinoxalin-6-amine (Intermediate 3B, 40 mg; 0.12 mmol; 1 eq.), sodium carbonate (61 mg; 0.58 mmol; 5 eq.), 2-methyl-5-(tetramethyl-1,3,2-dioxaborolan-2-yl)-1,3-benzothiazole (Intermediate 85, 35 mg; 0.13 mmol; 1.10 eq.), tetrakis(triphenylphosphine)palladium(0) (7.05 mg; 0.01 mmol; 0.05 eq.) in water (0.50 mL), ethanol (0.50 mL) and toluene (1 mL). Conditions: 110° C. overnight. Purification by reversed-phase preparative HPLC (column: Gemini NX C18 5u 110A (100×30 mm), ACN gradient in water). The pure fractions are pooled, MeCN is evaporated and the resulting solution is basified with NaHCO₃. It is then extracted with DCM (twice) and the combined organic layers are dried (sodium sulfate) and evaporated to dryness to yield N-(4-methanesulfonylpyridin-3-yl)-8-(2-methyl-1,3-benzothiazol-5-yl)quinoxalin-6-amine (18 mg; 0.04 mmol; 34%; yellow powder; HPLC purity: 99.8%).

### Example 170

### N-(4-Methanesulfonylpyridin-3-yl)-8-(1-methyl-1H-1,2,3-benzotriazol-6-yl)quinoxalin-6-amine

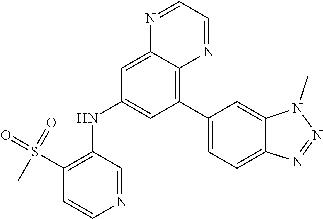 [SMILES: Cn1nnc2ccc(-c3cc(Nc4cnccc4S(C)(=O)=O)cc4nccnc34)cc21]

The title compound is prepared according to General Procedure 17 described in Example 66, using chloro-N-(4-methanesulfonylpyridin-3-yl)quinoxalin-6-amine (Intermediate 3B, 40 mg; 0.12 mmol; 1 eq.), sodium carbonate (61 mg; 0.58 mmol; 5 eq.), 1-methyl-6-(tetramethyl-1,3,2-dioxaborolan-2-yl)-1H-1,2,3-benzotriazole (Intermediate 82, 33 mg; 0.13 mmol; 1.10 eq.), tetrakis-(triphenylphosphine)palladium(0) (7 mg; 0.01 mmol; 0.05 eq) in water (0.40 mL), ethanol (0.40 mL) and toluene (0.8 mL). Conditions: 100° C. overnight. Purification by FCC (0% to 20% EtOAc gradient in hexane) followed by reversed-phase preparative HPLC (column: Gemini NX C18 5u 110A (100×30 mm), ACN gradient in water). The pure fractions are pooled, MeCN is evaporated and the resulting solution is basified with NaHCO₃. It is then extracted with DCM (twice) and the combined organic layers are dried (sodium sulfate) and evaporated to dryness to yield N-(4-methanesulfonyl-pyridin-3-yl)-8-(1-methyl-1H-1,2,3-benzotriazol-6-yl)quinoxalin-6-amine (22 mg; 0.05 mmol; 45%; brown yellow powder; HPLC purity: 100%).

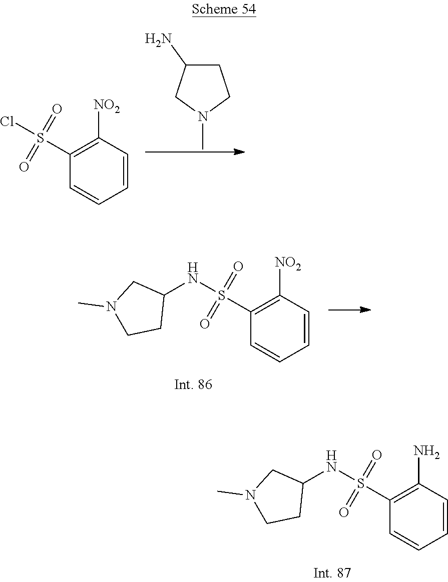

### Intermediate 86

### N-(1-Methylpyrrolidin-3-yl)-2-nitrobenzene-1-sulfonamide

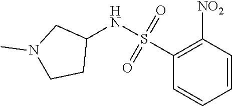 [SMILES: CN1CCC(NS(=O)(=O)c2ccccc2[N+](=O)[O-])C1]

The title compound is prepared according to General Procedure 24 described for Intermediate 83, using DIPEA (0.47 mL; 2.71 mmol; 3 eq.), 2-nitrobenzenesulfonyl chloride (200 mg; 0.90 mmol; 1 eq.) and 1-methylpyrrolidin-3-amine (0.15 mL; 1.35 mmol; 1.50 eq.) in anhydrous THF (10 mL). Conditions: room temperature for 16 h. The crude N-(1-methylpyrrolidin-3-yl)-2-nitrobenzene-1-sulfonamide (250 mg; 0.84 mmol;

93%; pale yellow oil; UPLC purity: 96%) is used in the next step without further purification.

### Intermediate 87

### 2-Amino-N-(1-methylpyrrolidin-3-yl)benzene-1-sulfonamide

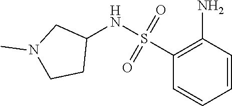 [SMILES: CN1CCC(NS(=O)(=O)c2ccccc2N)C1]

The title compound is prepared according to General Procedure 25 described for Intermediate 84, using iron powder (180 mg; 3.22 mmol; 4 eq.), N-(1-methylpyrrolidin-3-yl)-2-nitrobenzene-1-sulfonamide (Intermediate 86, 0.25 g; 0.81 mmol; 1 eq.) in acetic acid (2 mL), ethanol (2 mL) and water (1 mL). Conditions: 30° C. under sonication for 1 h. The crude 2-amino-N-(1-methylpyrrolidin-3-yl)benzene-1-sulfonamide (200 mg; 0.75 mmol; 93%; pale brown oil; UPLC purity: 95%) is used in the next step without further purification.

### Example 171

### 2-{[8-(1-Methyl-1H-indol-6-yl)quinoxalin-6-yl]amino}-N-(1-methylpyrrolidin-3-yl)benzene-1-sulfonamide

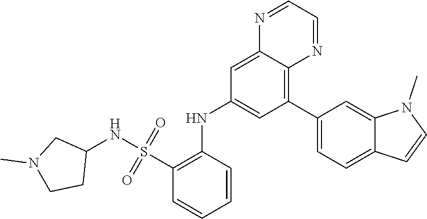 [SMILES: CN1CCC(NS(=O)(=O)c2ccccc2Nc2cc(-c3ccc4ccn(C)c4c3)c3nccnc3c2)C1]

The title compound is prepared according to General Procedure 1 described in Example 1, using 7-chloro-5-(1-methyl-1H-indol-6-yl)quinoxaline (Intermediate 2B, 150 mg; 0.50 mmol; 0.75 eq.), 2-amino-N-(1-methylpyrrolidin-3-yl)benzene-1-sulfonamide (Intermediate 87, 188 mg; 0.66 mmol; 1 eq.), cesium carbonate (1.08 g; 3.32 mmol; 5 eq.), BINAP (42 mg; 0.06 mmol; 0.10 eq.), palladium(II) acetate (14 mg; 0.06 mmol; 0.10 eq.) and anhydrous dioxane (2 mL). Conditions: 150° C. for 1.5 hours. Purification by FCC (0% to 5% MeOH gradient in DCM) affords 2-{[8-(1-methyl-1H-indol-6-yl)quinoxalin-6-yl]amino}-N-(1-methylpyrrolidin-3-yl)benzene-1-sulfonamide (37 mg; 0.07 mmol; 20%; brown solid; HPLC purity: 93.8%).

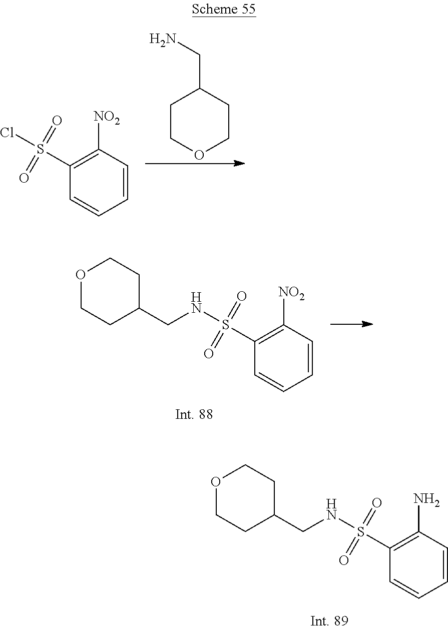

### Intermediate 88

### 2-Nitro-N-(oxan-4-ylmethyl)benzene-1-sulfonamide

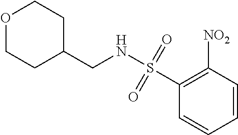 [SMILES: O=[N+]([O-])c1ccccc1S(=O)(=O)NCC1CCOCC1]

The title compound is prepared according to General Procedure 24 described for Intermediate 83, using TEA (0.25 mL; 1.80 mmol; 2 eq.), 2-nitrobenzenesulfonyl chloride (200 mg; 0.90 mmol; 1 eq.) and tetrahydro-pyran-4-yl-methylamine (125 mg; 1.08 mmol; 1.20 eq.) in DCM (2 mL). Conditions: room temperature for 24 h. The crude 2-nitro-N-(oxan-4-ylmethyl)benzene-1-sulfonamide (269 mg; 0.89 mmol; 99%; yellow oil; UPLC purity: 99%) is used in the next step without further purification.

### Intermediate 89

### 2-Amino-N-(oxan-4-ylmethyl)benzene-1-sulfonamide

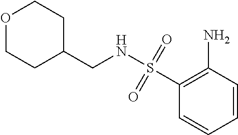 [SMILES: Nc1ccccc1S(=O)(=O)NCC1CCOCC1]

The title compound is prepared according to General Procedure 25 described for Intermediate 84, using iron powder (152 mg; 2.69 mmol; 3 eq.), 2-nitro-N-(oxan-4-ylmethyl)benzene-1-sulfonamide (Intermediate 88, 269 mg, 0.9 mmol, 1 eq.) in 35% aq. HCl (160 μl; 1.79 mmol; 2 eq.), ethanol (5 mL) and water (270 μl). Conditions: 40° C. under sonication for 2 h. The crude 2-amino-N-(oxan-4-ylmethyl)benzene-1-sulfonamide (194 mg; 0.68 mmol; 76%; UPLC purity: 95%) is used in the next step without further purification.

### Example 172

### 2-{[8-(1-Methyl-1H-indol-6-yl)quinoxalin-6-yl]amino}-N-[(oxan-4-yl)methyl]benzene-1-sulfonamide

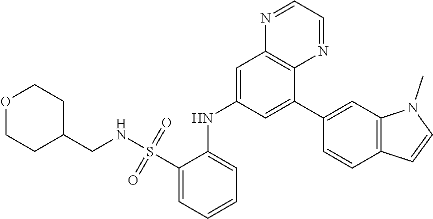 [SMILES: Cn1ccc2ccc(-c3cc(Nc4ccccc4S(=O)(=O)NCC4CCOCC4)cc4nccnc34)cc21]

The title compound is prepared according to General Procedure 1 described in Example 1, using 7-chloro-5-(1-methyl-1H-indol-6-yl)quinoxaline (Intermediate 2B, 127 mg; 0.42 mmol; 0.75 eq.), 2-amino-N-(oxan-4-ylmethyl)benzene-1-sulfonamide (Intermediate 89, 160 mg; 0.56 mmol; 1 eq.), cesium carbonate (917 mg; 2.82 mmol; 5 eq.), BINAP (35 mg; 0.06 mmol; 0.10 eq.), palladium(II) acetate (13 mg; 0.06 mmol; 0.10 eq.) and anhydrous dioxane (2 mL). Conditions: 150° C. for 1.5 hours. Purification by FCC (0% to 5% MeOH gradient in DCM) affords 2-{[8-(1-methyl-1H-indol-6-yl)quinoxalin-6-yl]amino}-N-[(oxan-4-yl)methyl]benzene-1-sulfonamide (38 mg; 0.07 mmol; 25%; yellow powder; HPLC purity: 96.9%).

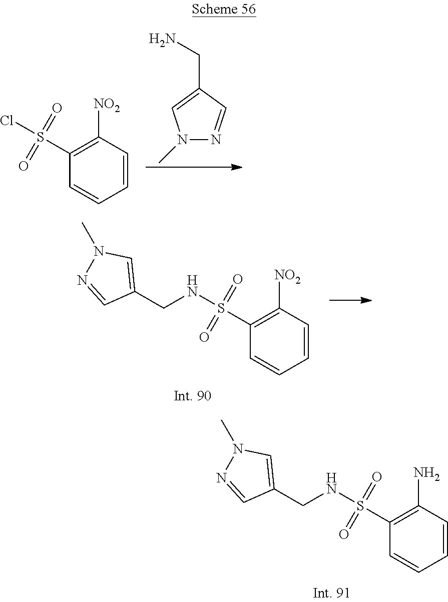

### Intermediate 90

### N-[(1-Methyl-1H-pyrazol-4-yl)methyl]-2-nitrobenzene-1-sulfonamide

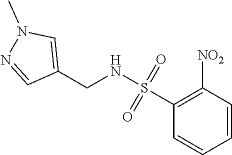 [SMILES: Cn1cc(CNS(=O)(=O)c2ccccc2[N+](=O)[O-])cn1]

The title compound is prepared according to General Procedure 24 described for Intermediate 83, using TEA (176 μl; 1.35 mmol; 3 eq.), 2-nitrobenzenesulfonyl chloride (100 mg; 0.45 mmol; 1 eq.) and 1-methyl-1H-pyrazol-4-yl-methylamine (60 mg; 0.54 mmol; 1.20 eq.) in DCM (6 mL). Purification by FCC (0% to 3% MeOH gradient in DCM) affords N-[(1-methyl-1H-pyrazol-4-yl)methyl]-2-nitrobenzene-1-sulfonamide (108 mg; 0.36 mmol; 80%; colorless gel; UPLC purity: 99%).

### Intermediate 91

### 2-Amino-N-[(1-methyl-1H-pyrazol-4-yl)methyl]benzene-1-sulfonamide

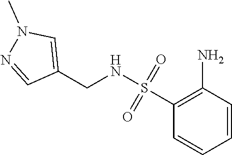 [SMILES: Cn1cc(CNS(=O)(=O)c2ccccc2N)cn1]

The title compound is prepared according to General Procedure 25 described for Intermediate 84, using iron powder (80 mg; 1.42 mmol; 4 eq.), N-[(1-methyl-1H-pyrazol-4-yl)methyl]-2-nitrobenzene-1-sulfonamide (Intermediate 90, 106 mg; 0.36 mmol; 1 eq.) in acetic acid (5 mL). Conditions: 80° C. for 2 h. Purification by FCC (0% to 3% MeOH gradient in DCM) affords 2-amino-N-[(1-methyl-1H-pyrazol-4-yl)methyl]benzene-1-sulfonamide (73 mg; 0.27 mmol; 77%; white semi-solid; UPLC purity: 90%).

### Example 173

### 2-{[8-(1-Methyl-1H-indol-6-yl)quinoxalin-6-yl]amino}-N-[(1-methyl-1H-pyrazol-4-yl)methyl]benzene-1-sulfonamide

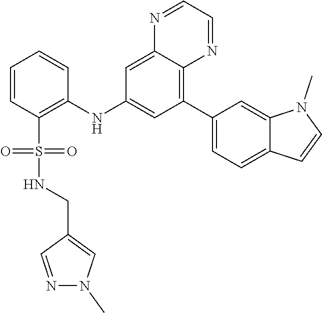 [SMILES: Cn1cc(CNS(=O)(=O)c2ccccc2Nc2cc(-c3ccc4ccn(C)c4c3)c3nccnc3c2)cn1]

The title compound is prepared according to General Procedure 1 described in Example 1, using 7-chloro-5-(1-methyl-1H-indol-6-yl)quinoxaline (Intermediate 2B, 62 mg; 0.21 mmol; 0.75 eq.), 2-amino-N-[(1-methyl-1H-pyrazol-4-yl)methyl]benzene-1-sulfonamide (Intermediate 91, 73 mg; 0.27 mmol; 1 eq.), cesium carbonate (446 mg; 1.37 mmol; 5 eq.), BINAP (17 mg; 0.03 mmol; 0.10 eq.), palladium(II) acetate (6 mg; 0.03 mmol; 0.10 eq.) and anhydrous dioxane (2 mL). Conditions: 150° C. for 1.5 hours. Purification by FCC (0% to 5% MeOH gradient in DCM) affords 2-{[8-(1-methyl-1H-indol-6-yl)quinoxalin-6-yl]amino}-N-[(1-methyl-1H-pyrazol-4-yl)methyl]benzene-1-sulfonamide (40 mg; 0.07 mmol; 27%; yellow powder; HPLC purity: 97.7%).

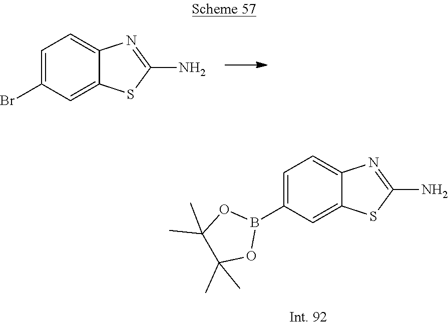 [SMILES: Nc1nc2ccc(Br)cc2s1 ; CC1(C)OB(c2ccc3nc(N)sc3c2)OC1(C)C]

### Intermediate 92

### 6-(Tetramethyl-1,3,2-dioxaborolan-2-yl)-1,3-benzothiazol-2-amine

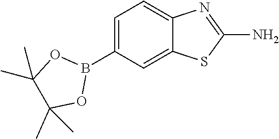 [SMILES: CC1(C)OB(c2ccc3nc(N)sc3c2)OC1(C)C]

The title compound is prepared according to General Procedure 8 described for Intermediate 14, using 6-bromo-benzothiazol-2-ylamine (1 g; 4.36 mmol; 1 eq.), bis(pinacolato)diboron (1.66 g; 6.55 mmol; 1.50 eq.), potassium acetate (0.94 g; 9.60 mmol; 2.20 eq.) and Pd(dppf)Cl₂ (363 mg; 0.44 mmol; 0.10 eq.) in dioxane (10 mL). Purification by FCC (30% EtOAc in hexane) affords 6-(tetramethyl-1,3,2-dioxaborolan-2-yl)-1,3-benzothiazol-2-amine (339 mg; 0.92 mmol; 21%; white solid; UPLC purity: 75%).

### Example 174

### 8-(2-Amino-1,3-benzothiazol-6-yl)-N-(4-methanesulfonylpyridin-3-yl)quinoxalin-6-amine

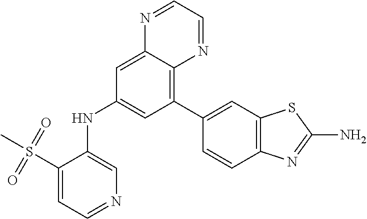 [SMILES: CS(=O)(=O)c1ccncc1Nc1cc(-c2ccc3nc(N)sc3c2)c2nccnc2c1]

The title compound is prepared according to General Procedure 17 described in Example 66, using chloro-N-(4-methanesulfonylpyridin-3-yl)quinoxalin-6-amine (Intermediate 3B, 150 mg; 0.43 mmol; 1 eq.), sodium carbonate (230 mg; 2.17 mmol; 5 eq.), 6-(tetramethyl-1,3,2-dioxaborolan-2-yl)-1,3-benzothiazol-2-amine (Intermediate 92, 176 mg; 0.48 mmol; 1.10 eq.), tetrakis(triphenylphosphine)palladium(0) (26 mg; 0.02 mmol; 0.05 eq.) in water (1 mL), ethanol (1 mL) and toluene (2 mL). Conditions: 110° C. overnight. Purification by trituration in DCM followed by FCC (0% to 10% MeOH gradient in EtOAc) yields 8-(2-amino-1,3-benzothiazol-6-yl)-N-(4-methanesulfonylpyridin-3-yl)quinoxalin-6-amine (163 mg; 0.36 mmol; 84%; yellow powder; HPLC purity: 100%).

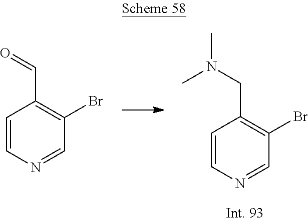

### Intermediate 93, General Procedure 26

### [(3-Bromopyridin-4-yl)methyl]dimethylamine

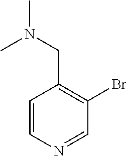

Hantzsch ester (351 mg; 1.32 mmol; 1.25 eq.) and chlorotrimethylsilane (55 μl; 0.42 mmol; 0.40 eq.) are added to a stirred solution of 3-bromopyridine-4-carbaldehyde (200 mg; 1.05 mmol; 1 eq.) in DCE (10 mL). The reaction mixture is stirred for 6 h at room temperature, poured in stirring saturated aqueous NaHCO₃ and extracted with DCM. The organic layer is washed with water, dried over Na₂SO₄ and filtered through a pad of celite. The filtrate is concentrated in vacuo to a residue which is purified by FCC (0% to 50% EtOAc gradient in hexane) to afford [(3-bromopyridin-4-yl)methyl]-dimethylamine (95 mg; 0.42 mmol; 40%; yellow oil; UPLC purity: 96%).

### Example 175

### N-{4-[(dimethylamino)methyl]pyridin-3-yl}-8-(1-methyl-1H-indol-6-yl)quinoxalin-6-amine

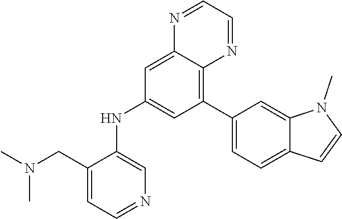 [SMILES: CN(C)Cc1ccncc1Nc1cc(-c2ccc3ccn(C)c3c2)c2nccnc2c1]

The title compound is prepared according to General Procedure 1 described in Example 1, using 8-(1-methyl-1H-indol-6-yl)quinoxalin-6-amine (Intermediate 4, 60 mg; 0.21 mmol; 1 eq.), [(3-bromopyridin-4-yl)methyl]dimethylamine (Intermediate 93, 53 mg; 0.23 mmol; 1.10 eq.), cesium carbonate (175 mg; 0.53 mmol; 2.50 eq.), BINAP (27 mg; 0.04 mmol; 0.20 eq.), palladium(II) acetate (10 mg; 0.04 mmol; 0.20 eq.) and anhydrous dioxane (2 mL). Conditions: 150° C. for 1.5 hours. Purification by FCC (0% to 100% EtOAc gradient in hexane followed by 0% to 5% MeOH gradient in EtOAc) affords N-{4-[(dimethylamino)methyl]pyridin-3-yl}-8-(1-methyl-1H-indol-6-yl)quinoxalin-6-amine (55 mg; 0.13 mmol; 61%; yellow powder; HPLC purity: 97.1%).

### Example 176

### N-{2-[(Dimethylamino)methyl]phenyl}-8-(1-methyl-1H-indol-6-yl)quinoxalin-6-amine

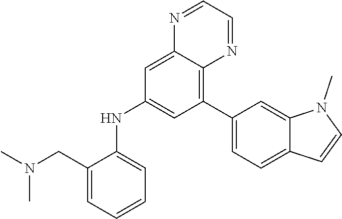 [SMILES: CN(C)Cc1ccccc1Nc1cc(-c2ccc3ccn(C)c3c2)c2nccnc2c1]

The title compound is prepared according to General Procedure 2 described in Example 6, using 7-chloro-5-(1-methyl-1H-indol-6-yl)-quinoxaline (Intermediate 2B, 50 mg; 0.17 mmol; 1 eq.), 2-dimethylaminomethyl-phenylamine (38 mg; 0.26 mmol; 1.50 eq.), tBuONa (65 mg; 0.68 mmol; 4 eq.), Pd₂(dba)₃ (16 mg; 0.02 mmol; 0.10 eq.), BINAP (21 mg; 0.03 mmol; 0.20 eq.) and toluene (1 mL). Purification by FCC (0% to 5% MeOH gradient in DCM) affords N-{2-[(dimethylamino)methyl]phenyl}-8-(1-methyl-1H-indol-6-yl)quinoxalin-6-amine (15 mg; 0.04 mmol; 22%; yellow powder; HPLC purity: 99.9%).

### Example 178

### 2-{[8-(1-Methyl-1H-indol-6-yl)quinoxalin-6-yl]amino}benzoic acid

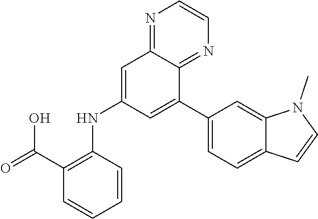 [SMILES: Cn1ccc2ccc(-c3cc(Nc4ccccc4C(=O)O)cc4nccnc34)cc21]

2-{[8-(1-Methyl-1H-indol-6-yl)quinoxalin-6-yl]amino}benzonitrile (Example 88, 80 mg; 0.21 mmol; 1 eq.) and iPrOH (0.50 mL) are added to a solution of KOH (295 mg; 5.26 mmol; 25 eq.) in water (2 mL) and the mixture is refluxed for 32 h. After coming back to room temperature, the reaction mixture is diluted with n-ButOH and neutralized with 1M HCl. The phases are separated and the aqueous phase is extracted with n-ButOH. The combined organic layers are dried over Na₂SO₄, filtered and evaporated in vacuo. The residue is dissolved in absolute EtOH (5 mL). To this solution, incrementing amounts of DCM (around 30 mL in total) are added under stirring. The resulting precipitate, mostly containing inorganic impurities, is filtered off over a pad of celite and the filtrate is evaporated to dryness to yield 2-{[8-(1-methyl-1H-indol-6-yl)quinoxalin-6-yl]amino}benzoic acid (80 mg; 0.19 mmol; 91%; yellow powder; HPLC purity: 94.2%).

### Example 179

### 3-{[8-(1-Methyl-1H-indol-6-yl)quinoxalin-6-yl]amino}pyridine-4-carboxylic acid

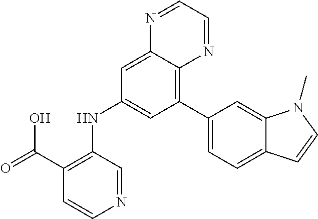 [SMILES: Cn1ccc2ccc(-c3cc(Nc4cnccc4C(=O)O)cc4nccnc34)cc21]

Crude 3-{[8-(1-Methyl-1H-indol-6-yl)quinoxalin-6-yl]amino}pyridine-4-carboxylic acid (Intermediate 42, 1.50 g; 3.80 mmol; 1 eq.) is dissolved in absolute EtOH (10 mL). To this solution, incrementing amounts of DCM (50 mL in total) are added under stirring. The resulting precipitate, mostly containing inorganic impurities, is filtered off over a pad of celite and the filtrate is evaporated to dryness to yield 3-{[8-(1-methyl-1H-indol-6-yl)quinoxalin-6-yl]amino}pyridine-4-carboxylic acid (1.05 g; 2.66 mmol; 70%; yellow powder; HPLC purity: 96%).

### Example 181

### 3-{[8-(1-Methyl-1H-indol-6-yl)quinoxalin-6-yl]amino}-N-(1-methylazetidin-3-yl)pyridine-4-carboxamide

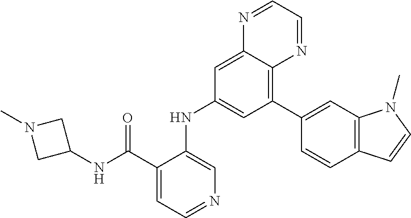 [SMILES: CN1CC(NC(=O)c2ccncc2Nc2cc(-c3ccc4ccn(C)c4c3)c3nccnc3c2)C1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 80 mg; 0.20 mmol; 1 eq.), 1-methylazetidin-3-ylamine hydrochloride (41 mg; 0.25 mmol; 1.25 eq.), EDC.HCl (69 mg; 0.35 mmol; 1.80 eq.), HOBt hydrate (55 mg; 0.35 mmol; 1.80 eq.), triethylamine (0.13 mL; 0.98 mmol; 5 eq.) and dioxane (5 mL). Conditions: room temperature for 24 h. Purification by FCC (60% to 100% DCM gradient in hexane followed by 0% to 10% MeOH gradient in DCM) followed by preparative HPLC (column: Gemini NX C18 5u 110A (100×30 mm), ACN gradient in water) yields 3-{[8-(1-methyl-1H-indol-6-yl)quinoxalin-6-yl]amino}-N-(1-methylazetidin-3-yl)pyridine-4-carboxamide as its TFA salt (15 mg; 0.02 mmol; 13%; yellow orange powder; HPLC purity: 84.6%).

### Example 182

### N-Methyl-3-{[8-(1-methyl-1H-indol-6-yl)quinoxalin-6-yl]amino}-N-(1-methylpyrrolidin-3-yl)pyridine-4-carboxamide

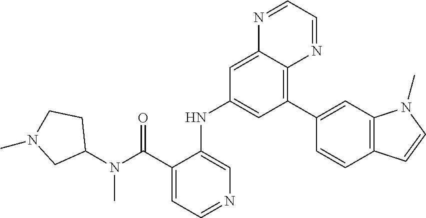 [SMILES: CN1CCC(N(C)C(=O)c2ccncc2Nc2cc(-c3ccc4ccn(C)c4c3)c3nccnc3c2)C1]

The title compound is prepared according to General Procedure 13 described in Example 52, using 3-{[8-(1-methyl-1H-indol-6-yl)quinoxalin-6-yl]amino}pyridine-4-carboxylic acid (Intermediate 42, 60 mg; 0.15 mmol; 1 eq.), methyl-(1-methylpyrrolidin-3-yl)-amine (22 mg; 0.18 mmol; 1.25 eq.), EDC.HCl (52 mg; 0.26 mmol; 1.80 eq.), HOBt hydrate (35 mg; 0.26 mmol; 1.80 eq.), triethylamine (0.10 mL; 0.74 mmol; 5 eq.) and dioxane (5 mL). Conditions: room temperature for 24 h. Purification by FCC (silica deactivated with ammonia in DCM, 0% to 10% MeOH gradient in DCM) yields N-methyl-3-{[8-(1-methyl-1H-indol-6-yl)quinoxalin-6-yl]amino}-N-(1-methylpyrrolidin-3-yl)pyridine-4-carboxamide (64 mg; 0.12 mmol; 84.9%; yellow powder; HPLC purity: 96%).

### Example 183

### 2-{[8-(1-Methyl-1H-indol-6-yl)quinoxalin-6-yl]amino}-N-(1-methylpyrrolidin-3-yl)benzamide

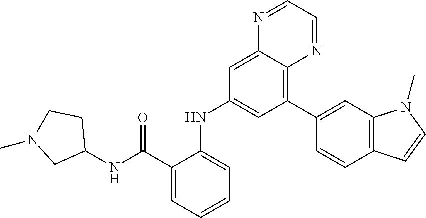 [SMILES: CN1CCC(NC(=O)c2ccccc2Nc2cc(-c3ccc4ccn(C)c4c3)c3nccnc3c2)C1]

The title compound is prepared according to General Procedure 13 described in Example 52, 2-{[8-(1-methyl-1H-indol-6-yl)quinoxalin-6-yl]amino}benzoic acid (Example 178, 60 mg; 0.15 mmol; 1 eq.), 1-methylpyrrolidin-3-ylamine hydrochloride (22 mg; 0.12 mmol; 1.25 eq.), EDC.HCl (34 mg; 0.17 mmol; 1.80 eq.), HOBt hydrate (27 mg; 0.17 mmol; 1.80 eq.), triethylamine (0.06 mL; 0.48 mmol; 5 eq.) and dioxane (5 mL). Conditions: room temperature for 24 h. Purification by FCC (silica deactivated with ammonia in DCM, 0% to 10% MeOH gradient in DCM) yields 2-{[8-(1-methyl-1H-indol-6-yl)quinoxalin-6-yl]amino}-N-(1-methylpyrrolidin-3-yl)benzamide (36 mg; 0.07 mmol; 73%; yellow powder; HPLC purity: 92.8%).

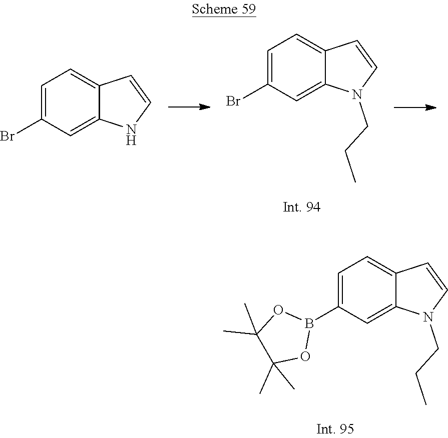 [SMILES: Brc1ccc2cc[nH]c2c1 ; CCCn1ccc2ccc(Br)cc21 ; CCCn1ccc2ccc(B3OC(C)(C)C(C)(C)O3)cc21]

### Intermediate 94

### 6-Bromo-1-propyl-1H-indole

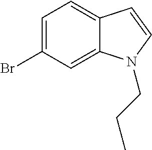

The title compound is prepared according to General Procedure 14 described for Intermediate 45, using 6-bromo-1H-indole (300 mg; 1.53 mmol; 1 eq.), NaH (60% in mineral oil, 92 mg; 2.30 mmol; 1.50 eq.), 1-iodopropane (312 mg; 1.84 mmol; 1.20 eq.) in dry THF (5 mL). Conditions: 0° C. to room temperature for 15 h. The crude 6-bromo-1-propyl-1H-indole (338 mg; 1.21 mmol; 79%; yellow oil; UPLC purity: 85%) is used in the next step without further purification.

### Intermediate 95

### 1-Propyl-6-(tetramethyl-1,3,2-dioxaborolan-2-yl)-1H-indole

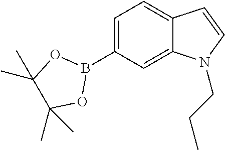 [SMILES: CCCn1ccc2ccc(B3OC(C)(C)C(C)(C)O3)cc21]

The title compound is prepared according to General Procedure 8 described for Intermediate 14, using 6-bromo-1-propyl-1H-indole (Intermediate 94, 338 mg; 1.42 mmol; 1 eq.), bis(pinacolato)diboron (469 mg; 1.85 mmol; 1.30 eq.), potassium acetate (279 mg; 2.84 mmol; 2 eq.) and Pd(dppf)Cl₂ (52 mg; 0.07 mmol; 0.05 eq.) in dioxane (3 mL). Conditions: 100° C. overnight. Purification by FCC (0% to 5% EtOAc gradient in hexane) affords 1-propyl-6-(tetramethyl-1,3,2-dioxaborolan-2-yl)-1H-indole (242 mg; 0.63 mmol; 44%; white solid; UPLC purity: 74%).

### Example 184

### N-(4-Methanesulfonylpyridin-3-yl)-8-(1-propyl-1H-indol-6-yl)quinoxalin-6-amine

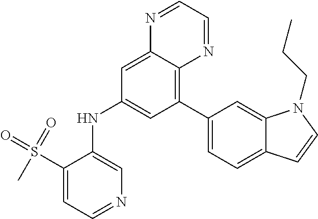 [SMILES: CCCn1ccc2ccc(-c3cc(Nc4cnccc4S(C)(=O)=O)cc4nccnc34)cc21]

The title compound is prepared according to General Procedure 17 described in Example 66, using chloro-N-(4-methanesulfonylpyridin-3-yl)quinoxalin-6-amine (Intermediate 3B, 100 mg; 0.29 mmol; 1 eq.), sodium carbonate (154 mg; 1.45 mmol; 5 eq.), 1-propyl-6-(tetramethyl-1,3,2-dioxaborolan-2-yl)-1H-indole (Intermediate 95, 121 mg; 0.32 mmol; 1.10 eq.), tetrakis(triphenylphosphine)palladium(0) (18 mg; 0.01 mmol; 0.05 eq.) in water (1 mL), ethanol (1 mL) and toluene (2 mL). Conditions: 110° C. overnight. Purification by FCC (50% to 100% EtOAc gradient in hexane) yields N-(4-methanesulfonylpyridin-3-yl)-8-(1-propyl-1H-indol-6-yl)quinoxalin-6-amine (105 mg; 0.21 mmol; 71%; yellow powder; HPLC purity: 90.2%).

### Example 185

### N-(4-Methanesulfonylpyridin-3-yl)-8-[4-(trifluoromethyl)phenyl]quinoxalin-6-amine

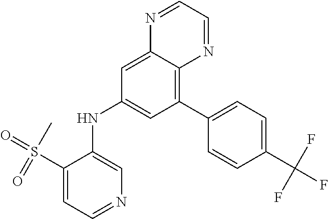 [SMILES: CS(=O)(=O)c1ccncc1Nc1cc(-c2ccc(C(F)(F)F)cc2)c2nccnc2c1]

The title compound is prepared according to General Procedure 17 described in Example 66, using chloro-N-(4-methanesulfonylpyridin-3-yl)quinoxalin-6-amine (Intermediate 3B, 40 mg; 0.12 mmol; 1 eq.), sodium carbonate (61 mg; 0.58 mmol; 5 eq.), [4-(trifluoromethyl)phenyl]boronic acid (42 mg; 0.22 mmol; 1.9 eq.), tetrakis(triphenylphosphine)palladium(0) (7 mg; 0.01 mmol; 0.05 eq.) in water (0.50 mL), ethanol (0.50 mL) and toluene (1 mL). Conditions: 110° C. for 6.5 hours. Purification by FCC (0% to 20% acetone gradient in DCM) followed by reversed-phase preparative HPLC (column: Gemini NX C18 5u 110A (100×30 mm), ACN gradient in water The pure fractions are pooled, MeCN is evaporated and the resulting solution is basified with 2M NaOH. It is then extracted with DCM (twice) and the combined organic layers are dried (sodium sulfate) and evaporated to dryness to yield N-(4-methanesulfonylpyridin-3-yl)-8-[4-(trifluoromethyl)phenyl]quinoxalin-6-amine (11 mg; 0.02 mmol; 21%; yellow powder; HPLC purity: 99.9%).

### Example 186

### 8-(4-Amino-3-fluorophenyl)-N-(4-methanesulfonylpyridin-3-yl)quinoxalin-6-amine

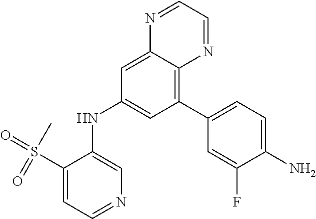 [SMILES: CS(=O)(=O)c1ccncc1Nc1cc(-c2ccc(N)c(F)c2)c2nccnc2c1]

The title compound is prepared according to General Procedure 17 described in Example 66, using chloro-N-(4-methanesulfonylpyridin-3-yl)quinoxalin-6-amine (Intermediate 3B, 100 mg; 0.26 mmol; 1 eq.), sodium carbonate (140 mg; 1.32 mmol; 5 eq.), 2-fluoro-4-(tetramethyl-1,3,2-dioxaborolan-2-yl)aniline (94 mg; 0.40 mmol; 1.50 eq.), tetrakis(triphenylphosphine)palladium(0) (16 mg; 0.01 mmol; 0.05 eq.) in water (0.50 mL), ethanol (0.50 mL) and toluene (1 mL). Conditions: 110° C. overnight. Purification by FCC (30% acetone in DCM) yields 8-(4-amino-3-fluorophenyl)-N-(4-methanesulfonylpyridin-3-yl)quinoxalin-6-amine (106 mg; 0.26 mmol; 98%; yellow solid; HPLC purity: 99.9%).

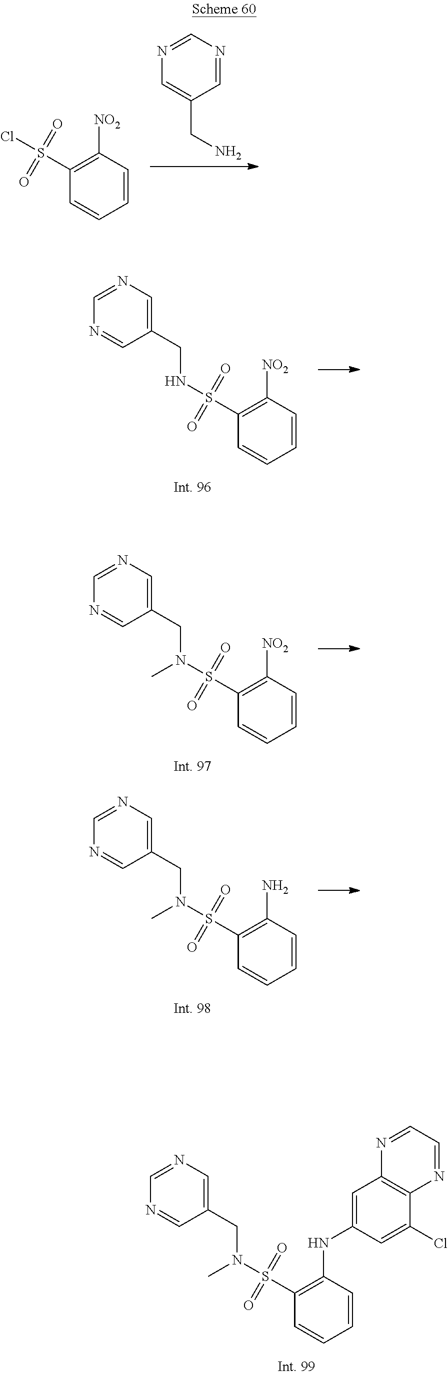 [SMILES: O=[N+]([O-])c1ccccc1S(=O)(=O)Cl ; NCc1cncnc1 ; O=[N+]([O-])c1ccccc1S(=O)(=O)NCc1cncnc1 ; CN(Cc1cncnc1)S(=O)(=O)c1ccccc1[N+](=O)[O-] ; CN(Cc1cncnc1)S(=O)(=O)c1ccccc1N ; CN(Cc1cncnc1)S(=O)(=O)c1ccccc1Nc1cc(Cl)c2nccnc2c1]

### Intermediate 96

### 2-Nitro-N-(pyrimidin-5-ylmethyl)benzene-1-sulfonamide

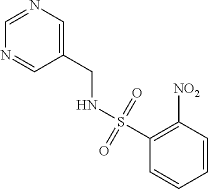 [SMILES: O=[N+]([O-])c1ccccc1S(=O)(=O)NCc1cncnc1]

The title compound is prepared according to General Procedure 24 described for Intermediate 83, using TEA (0.13 mL; 0.92 mmol; 1 eq.), 2-nitrobenzenesulfonyl chloride (203 mg; 0.92 mmol; 1 eq.) and pyrimidin-5-yl-methylamine (100 mg; 0.92 mmol; 1 eq.) in DCM (5 mL). Conditions: room temperature for 1.5 hours. The crude 2-nitro-N-(pyrimidin-5-ylmethyl)benzene-1-sulfonamide (0.25 g; 0.78 mmol; 85%; UPLC purity: 91%) is used in the next step without further purification.

### Intermediate 97

### N-Methyl-2-nitro-N-(pyrimidin-5-ylmethyl)benzene-1-sulfonamide

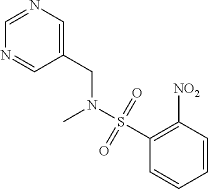 [SMILES: CN(Cc1cncnc1)S(=O)(=O)c1ccccc1[N+](=O)[O-]]

The title compound is prepared according to General Procedure 21 described in Example 104, using 2-nitro-N-(pyrimidin-5-ylmethyl)benzene-1-sulfonamide (Intermediate 96, 0.25 g; 0.78 mmol; 1 eq.), NaH (60% in mineral oil, 62 mg; 1.55 mmol; 2 eq.), iodomethane (0.11 mL; 1.55 mmol; 2 eq.), in anhydrous DMF (5 mL). Conditions: room temperature for 1 h. The crude N-methyl-2-nitro-N-(pyrimidin-5-ylmethyl)benzene-1-sulfonamide (0.15 g; 0.46 mmol; 59%; yellow oil; UPLC purity: 96%) is used in the next step without further purification.

### Intermediate 98

### 2-Amino-N-methyl-N-(pyrimidin-5-ylmethyl)benzene-1-sulfonamide

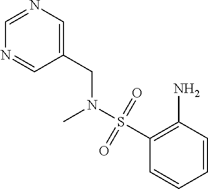 [SMILES: CN(Cc1cncnc1)S(=O)(=O)c1ccccc1N]

The title compound is prepared according to General Procedure 25 described for Intermediate 84, using iron powder (153 mg; 2.75 mmol; 6 eq.), N-methyl-2-nitro-N-(pyrimidin-5-ylmethyl)benzene-1-sulfonamide (Intermediate 97, 0.15 g; 0.46 mmol; 1 eq.), ammonium chloride (245 mg; 4.58 mmol; 10 eq.) in ethanol (15 mL). Conditions: reflux for 1.5 h. The crude 2-amino-N-methyl-N-(pyrimidin-5-ylmethyl)benzene-1-sulfonamide (111 mg; 0.39 mmol; 85%; beige oil; UPLC purity: 97%) is used in the next step without further purification.

### Intermediate 99

### 2-[(8-Chloroquinoxalin-6-yl)amino]-N-methyl-N-(pyrimidin-5-ylmethyl)benzene-1-sulfonamide

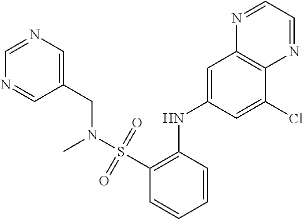 [SMILES: CN(Cc1cncnc1)S(=O)(=O)c1ccccc1Nc1cc(Cl)c2nccnc2c1]

The title compound is prepared according to General Procedure 2 described in Example 6, using 7-bromo-5-chloroquinoxaline (Intermediate 3, 32 mg; 0.13 mmol; 1 eq.), 2-amino-N-methyl-N-(pyrimidin-5-ylmethyl)benzene-1-sulfonamide (Intermediate 98, 38 mg; 0.13 mmol; 1 eq.), tBuONa (25 mg; 0.26 mmol; 2 eq.), Pd₂(dba)₃ (6 mg; 0.01 mmol; 0.05 eq.), BINAP (8 mg; 0.01 mmol; 0.10 eq.) and anhydrous dioxane (1 mL). Purification by FCC (0% to 5% MeOH gradient in DCM) yields 2-[(8-chloroquinoxalin-6-yl)amino]-N-methyl-N-(pyrimidin-5-ylmethyl)benzene-1-sulfonamide (29 mg; 0.05 mmol; 39%; UPLC purity: 79%).

### Example 187

### N-Methyl-2-{[8-(1-methyl-1H-indol-6-yl)quinoxalin-6-yl]amino}-N-[(pyrimidin-5-yl)methyl]benzene-1-sulfonamide

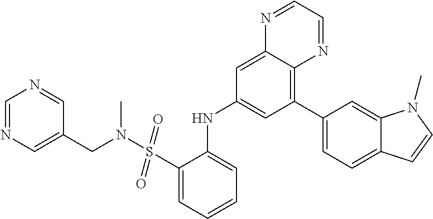 [SMILES: CN(Cc1cncnc1)S(=O)(=O)c1ccccc1Nc1cc(-c2ccc3ccn(C)c3c2)c2nccnc2c1]

2-[(8-Chloroquinoxalin-6-yl)amino]-N-methyl-N-(pyrimidin-5-ylmethyl)benzene-1-sulfonamide (Intermediate 99, 29 mg; 0.07 mmol; 1 eq.), 1-methyl-6-(tetramethyl-1,3,2-dioxaborolan-2-yl)-1H-indole (22 mg; 0.08 mmol; 1.20 eq.) and sodium carbonate (10 mg; 0.10 mmol; 1.50 eq.) are placed in a microwave vessel containing dioxane (1 mL) and water (1 mL). The resulting mixture is sparged with argon for 10 min and Pd(dppf)Cl₂ (5 mg; 0.01 mmol; 0.10 eq.) is added. The vessel is sealed and the reaction mixture is stirred at 140° C. under microwave irradiation for 90 min. After coming back to room temperature, it is then diluted with EtAOc and flitered through celite. The filtrate is washed with water and brine, dried over Na₂SO₄ and evaporated. The residue is purified by FCC (0% to 100% EtOAc gradient in hexane) and preparative HPLC. The pure fractions are pooled, MeCN is evaporated and the resulting solution is basified with NaHCO₃. It is then extracted with DCM (twice) and the combined organic layers are dried (sodium sulfate) and evaporated to dryness to yield N-methyl-2-{[8-(1-methyl-1H-indol-6-yl)quinoxalin-6-yl]amino}-N-[(pyrimidin-5-yl)methyl]benzene-1-sulfonamide formate salt (7 mg; 0.01 mmol; 17%; light yellow solid; HPLC purity: 99.5%).

### Example 188

### 8-(4-Fluorophenyl)-N-(4-methanesulfonylpyridin-3-yl)quinoxalin-6-amine

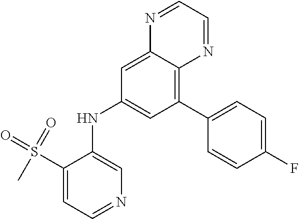 [SMILES: CS(=O)(=O)c1ccncc1Nc1cc(-c2ccc(F)cc2)c2nccnc2c1]

The title compound is prepared according to General Procedure 17 described in Example 66, using chloro-N-(4-methanesulfonylpyridin-3-yl)quinoxalin-6-amine (Intermediate 3B, 100 mg; 0.29 mmol; 1 eq.), sodium carbonate (154 mg; 1.45 mmol; 5 eq.), (4-fluorophenyl)boronic acid (65 mg; 0.35 mmol; 1.20 eq.), tetrakis(triphenylphosphine)palladium(0) (18 mg; 0.01 mmol; 0.05 eq.) in water (1 mL), ethanol (1 mL) and toluene (2 mL). Conditions: 110° C. overnight. Purification by FCC (50% to 100% EtOAc gradient in hexane) yields 8-(4-fluorophenyl)-N-(4-methanesulfonylpyridin-3-yl)quinoxalin-6-amine (85 mg; 0.21 mmol; 72%; yellow powder; HPLC purity: 97.4%).

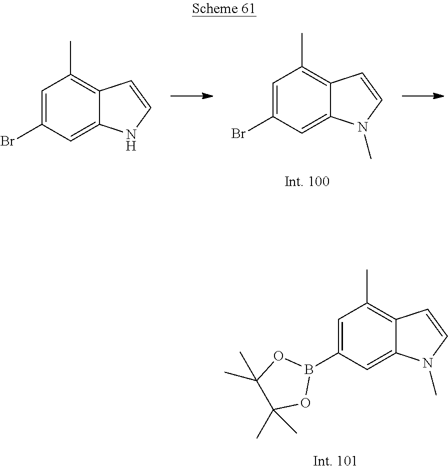 [SMILES: Cc1cc(Br)cc2[nH]ccc12 ; Cc1cc(Br)cc2c1ccn2C ; Cc1cc(B2OC(C)(C)C(C)(C)O2)cc2c1ccn2C]

### Intermediate 100

### 6-Bromo-1,4-dimethyl-1H-indole

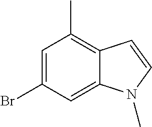

The title compound is prepared according to General Procedure 14 described for Intermediate 45, using 6-bromo-4-methyl-1H-indole (250 mg; 1.19 mmol; 1 eq.), NaH (60% in mineral oil, 71 mg; 1.79 mmol; 1.50 eq.), iodomethane (203 mg; 1.43 mmol; 1.20 eq.) in dry THF (5 mL). Conditions: 0° C. to room temperature for 15 h. The crude 6-bromo-1,4-dimethyl-1H-indole (227 mg; 1 mmol; 91%; yellow oil; UPLC purity: 87%) is used in the next step without further purification.

### Intermediate 101

### 1,4-Dimethyl-6-(tetramethyl-1,3,2-dioxaborolan-2-yl)-1H-indole

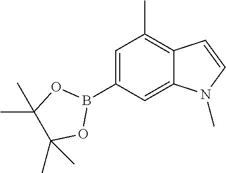 [SMILES: Cc1cc(B2OC(C)(C)C(C)(C)O2)cc2c1ccn2C]

The title compound is prepared according to General Procedure 8 described for Intermediate 14, using 6-bromo-1,4-dimethyl-1H-indole (Intermediate 100, 227 mg; 1.01 mmol; 1 eq.), bis(pinacolato)diboron (334 mg; 1.32 mmol; 1.30 eq.), potassium acetate (199 mg; 2.03 mmol; 2 eq.) and Pd(dppf)Cl₂ (37 mg; 0.05 mmol; 0.05 eq.) in dioxane (3 mL). Conditions: 100° C. overnight. Purification by FCC (gradient: 0% to 5% EtOAc in hexane) yields 1,4-dimethyl-6-(tetramethyl-1,3,2-dioxaborolan-2-yl)-1H-indole (223 mg; 0.82 mmol; 82%; white solid; UPLC purity: 74%).

### Example 189

### 8-(1,4-Dimethyl-1H-indol-6-yl)-N-(4-methanesulfonylpyridin-3-yl)quinoxalin-6-amine

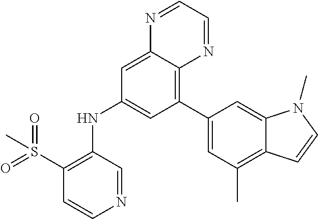 [SMILES: Cc1cc(-c2cc(Nc3cnccc3S(C)(=O)=O)cc3nccnc23)cc2c1ccn2C]

The title compound is prepared according to General Procedure 17 described in Example 66, using chloro-N-(4-methanesulfonylpyridin-3-yl)quinoxalin-6-amine (Intermediate 3B, 86 mg; 0.25 mmol; 1 eq.), sodium carbonate (132 mg; 1.24 mmol; 5 eq.), 1,4-dimethyl-6-(tetramethyl-1,3,2-dioxaborolan-2-yl)-1H-indole (Intermediate 101, 90 mg; 0.25 mmol; 1 eq.), tetrakis-(triphenylphosphine)palladium(0) (15 mg; 0.01 mmol; 0.05 eq.) in water (1 mL), ethanol (1 mL) and toluene (2 mL). Conditions: 110° C. overnight. Purification by FCC (50% to 100% EtOAc gradient in hexane) yields 8-(1,4-dimethyl-1H-indol-6-yl)-N-(4-methanesulfonylpyridin-3-yl)quinoxalin-6-amine (46 mg; 0.10 mmol; 40%; yellow powder; HPLC purity: 95.5%).

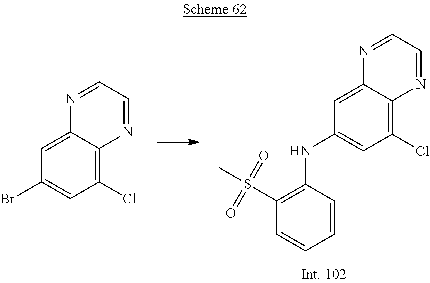 [SMILES: Clc1cc(Br)cc2nccnc12 ; CS(=O)(=O)c1ccccc1Nc1cc(Cl)c2nccnc2c1]

### Intermediate 102

### 8-Chloro-N-(2-methanesulfonylphenyl)quinoxalin-6-amine

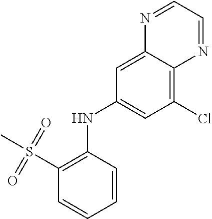 [SMILES: CS(=O)(=O)c1ccccc1Nc1cc(Cl)c2nccnc2c1]

The title compound is prepared according to General Procedure 1 described in Example 1, 7-bromo-5-chloroquinoxaline (Intermediate 3, 150 mg; 0.62 mmol; 1 eq.), 2-methanesulfonyl-phenylamine (127 mg; 0.74 mmol; 1.20 eq.), cesium carbonate (803 mg; 2.46 mmol; 4 eq.), BINAP (77 mg; 0.12 mmol; 0.20 eq.), palladium(II) acetate (14 mg; 0.06 mmol; 0.10 eq.) and anhydrous dioxane (10 mL). Conditions: 100° C. for 2 hours. Purification by FCC (0% to 10% MeOH gradient in DCM) affords 8-chloro-N-(2-methanesulfonylphenyl)quinoxalin-6-amine (160 mg; 0.44 mmol; 72%; yellow powder; UPLC purity: 63%).

### Example 190

### 8-(2-Amino-1,3-benzothiazol-5-yl)-N-(2-methanesulfonylphenyl)quinoxalin-6-amine

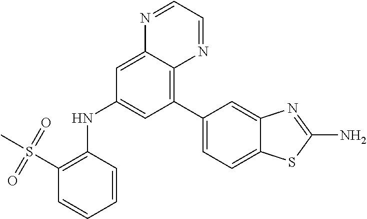 [SMILES: CS(=O)(=O)c1ccccc1Nc1cc(-c2ccc3sc(N)nc3c2)c2nccnc2c1]

The title compound is prepared according to General Procedure 17 described in Example 66, 8-chloro-N-(2-methanesulfonylphenyl)quinoxalin-6-amine (Intermediate 102, 80 mg; 0.24 mmol; 1 eq.), sodium carbonate (127 mg; 1.20 mmol; 5 eq.), 5-(tetramethyl-1,3,2-dioxaborolan-2-yl)-1,3-benzothiazol-2-amine (Intermediate 74, 73 mg; 0.26 mmol; 1.10 eq.), tetrakis(triphenylphosphine)palladium(0) (14 mg; 0.01 mmol; 0.05 eq.) in water (1 mL), ethanol (1 mL) and toluene (2 mL). Conditions: 100° C. overnight. Purification by FCC (0% to 10% MeOH gradient in DCM) and reversed-phase preparative HPLC (column: Gemini NX C18 5u 110A (100×30 mm), ACN gradient in water). The pure fractions are pooled, MeCN is evaporated and the resulting solution is basified with NaHCO₃. It is then extracted with DCM (twice) and the combined organic layers are dried (sodium sulfate) and evaporated to dryness to yield 8-(2-amino-1,3-benzothiazol-5-yl)-N-(2-methanesulfonylphenyl)quinoxalin-6-amine (40 mg; 0.09 mmol; 37%; yellow powder; HPLC purity: 99.2%).

### Example 191

### N-(2-Methanesulfonylphenyl)-8-(3-methyl-1-benzothiophen-5-yl)quinoxalin-6-amine

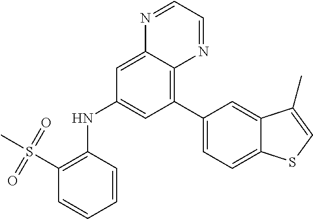 [SMILES: Cc1csc2ccc(-c3cc(Nc4ccccc4S(C)(=O)=O)cc4nccnc34)cc12]

A microwave vial is charged with 4,4,5,5-tetramethyl-2-(3-methyl-1-benzothiophen-5-yl)-1,3,2-dioxaborolane (69 mg; 0.25 mmol; 1.20 eq.), 8-chloro-N-(2-methanesulfonylphenyl)quinoxalin-6-amine (Intermediate 102, 70 mg; 0.21 mmol; 1 eq.) and sodium carbonate (33 mg; 0.31 mmol; 1.50 eq.). Dioxane (1.3 mL) and water (1.3 mL) are added, the mixture is sparged with argon for 10 min and the vial is sealed. The mixture is stirred at 140° C. under microwave irradiation for 90 min. After coming back to room temperature, it is filtered through celite, rinsing the filter cake with EtOAc and MeOH. The filtrate is concentrated in vacuo and the residue is partitioned between EtOAc and water. The aqueous phase is extracted with EtOAc (twice) and the combined organic layers are washed with brine, dried over Na₂SO₄, filtered and concentrated. The residue is purified by FCC (10% to 100% EtOAc gradient in hexane) to yield N-(2-methanesulfonylphenyl)-8-(3-methyl-1-benzothiophen-5-yl)quinoxalin-6-amine (18 mg; 0.04 mmol; 19%; pale brown solid; HPLC purity: 96.1%).

### Example 192

### 8-(3,5-Diethylphenyl)-N-(4-methanesulfonylpyridin-3-yl)quinoxalin-6-amine

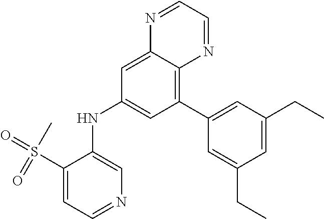 [SMILES: CCc1cc(CC)cc(-c2cc(Nc3cnccc3S(C)(=O)=O)cc3nccnc23)c1]

The title compound is prepared according to General Procedure 17 described in Example 66, using chloro-N-(4-methanesulfonylpyridin-3-yl)quinoxalin-6-amine (Intermediate 3B, 70 mg; 0.19 mmol; 1 eq.), sodium carbonate (98 mg; 0.93 mmol; 5 eq.), 2-(3,5-diethylphenyl)-4,4,5,5-tetramethyl-1,3,2-dioxaborolane (106 mg; 0.28 mmol; 1.50 eq.), tetrakis(triphenylphosphine)palladium(0) (11 mg; 0.01 mmol; 0.05 eq.) in water (0.50 mL), ethanol (0.50 mL) and toluene (1 mL). Conditions: 100° C. for 6 hours. Purification by FCC (50% to 100% EtOAc gradient in hexane) yields 8-(3,5-diethylphenyl)-N-(4-methanesulfonylpyridin-3-yl)quinoxalin-6-amine (19 mg; 0.04 mmol; 23%; white powder; HPLC purity: 99%).

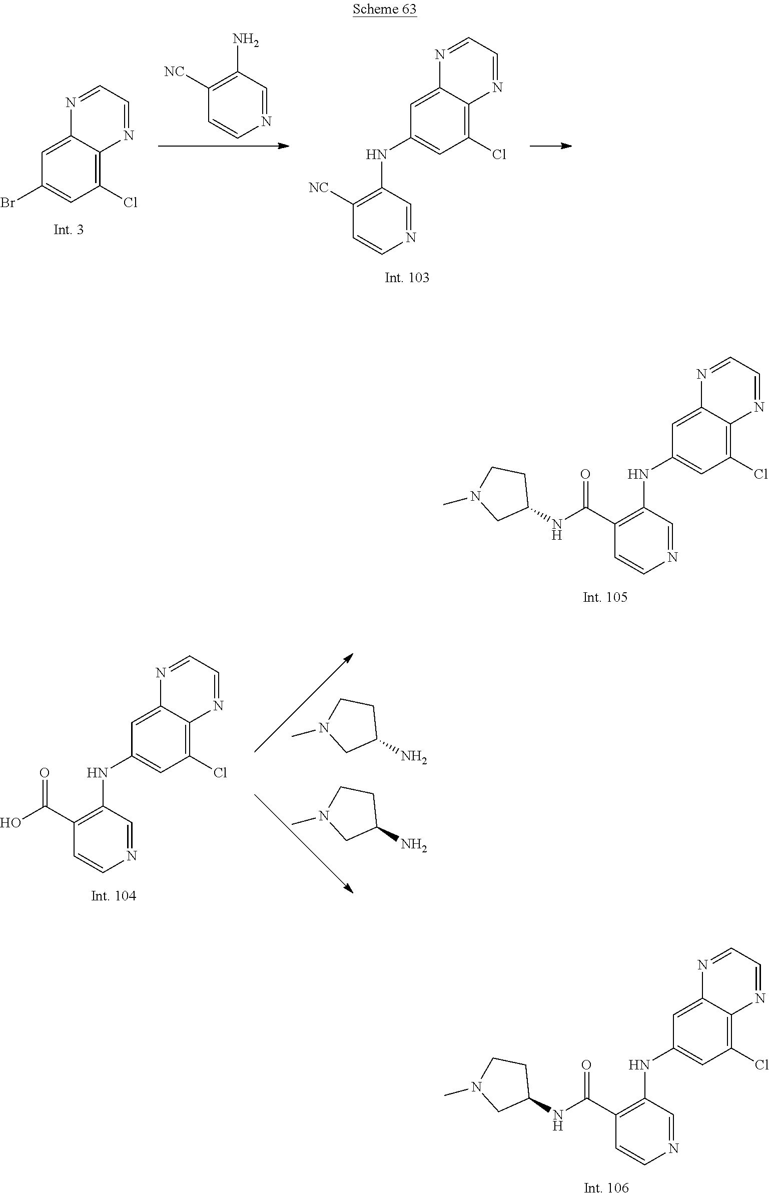

### Intermediate 103

### 3-[(8-Chloroquinoxalin-6-yl)amino]pyridine-4-carbonitrile

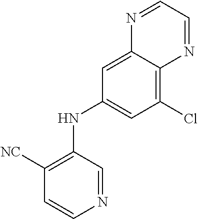 [SMILES: N#Cc1ccncc1Nc1cc(Cl)c2nccnc2c1]

The title compound is prepared according to General Procedure 1 described in Example 1, using 7-bromo-5-chloroquinoxaline (Intermediate 3, 1 g; 4.11 mmol; 1 eq.), 3-aminoisonicotinonitrile (0.49 g; 4.11 mmol; 1 eq.), cesium carbonate (5 g; 16.43 mmol; 4 eq.), BINAP (0.51 g; 0.82 mmol; 0.20 eq.), palladium(II) acetate (92 mg; 0.41 mmol; 0.10 eq.) and anhydrous dioxane (20 mL). Conditions: 110° C. for 3 hours. Purification by filtration through a pad of neutral allumina (20 g). The impolar impurities are eluted using DCM, and the expected compound is recovered using EtOAc as eluent. The EtOAc filtrate is evaporated to a residue which is triturated in the minimum amount of acetone (around 10 mL), filtered and dried under vacuum to give 3-[(8-chloroquinoxalin-6-yl)amino]pyridine-4-carbonitrile (540 mg; 1.80 mmol; 44%; yellow powder; UPLC purity: 94%).

### Intermediate 104

### 3-[(8-chloroquinoxalin-6-yl)amino]pyridine-4-carboxylic acid

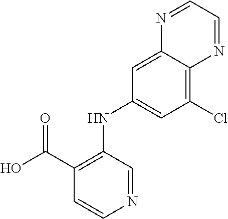 [SMILES: O=C(O)c1ccncc1Nc1cc(Cl)c2nccnc2c1]

In a pressure vessel, a solution of KOH (5.34 g; 76.12 mmol; 25 eq.) in water (60 mL) is added to a suspension 3-[(8-chloroquinoxalin-6-yl)amino]pyridine-4-carbonitrile (Intermediate 103, 0.90 g; 3.04 mmol; 1 eq.) in iPrOH (20 mL). The vessel is sealed, and the mixture is stirred at 115° C. for 1.5 h. After coming back to room temperature, the reaction mixture is acidified to pH 5 with 12N HCl. The obtained precipitate is filtered-off, washed with water, MeOH and Et₂O, and dried in vacuo. The crude 3-[(8-chloroquinoxalin-6-yl)amino]pyridine-4-carboxylic acid (914 mg; 2.64 mmol; 87%; yellow solid; UPLC purity: 87%) is used in next step without further purification.

### Intermediate 105

### 3-[(8-Chloroquinoxalin-6-yl)amino]-N-[(3S)-1-methylpyrrolidin-3-yl]pyridine-4-carboxamide

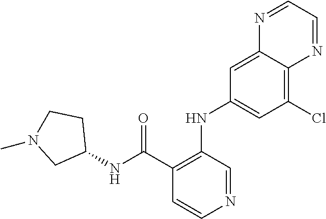 [SMILES: CN1CC[C@H](NC(=O)c2ccncc2Nc2cc(Cl)c3nccnc3c2)C1]

The title compound is prepared according to General Procedure 22 described in Example 126, using 3-[(8-chloroquinoxalin-6-yl)amino]pyridine-4-carboxylic acid (Intermediate 104, 100 mg; 0.32 mmol; 1 eq.), HATU (184 mg; 0.48 mmol; 1.50 eq.), DIPEA (0.18 mL; 1.29 mmol; 4 eq.), (S)-1-methylpyrrolidin-3-ylamine (0.07 mL; 0.65 mmol; 2 eq.) in anhydrous DMF (2.50 mL). Conditions: 50° C. for 2 h. Purification by FCC (30% to 40% MeOH gradient in DCM). The fractions containing the pure product are evaporated, the residue is redissolved in 3 mL of DCM and the solution is filtered on a 0.45 μm syringe filter and evaporated to dryness to yield 3-[(8-chloroquinoxalin-6-yl)amino]-N-[(3S)-1-methylpyrrolidin-3-yl]pyridine-4-carboxamide (113 mg; 0.28 mmol; 87%; yellow solid; UPLC purity: 95%).

### Intermediate 106

### 3-[(8-Chloroquinoxalin-6-yl)amino]-N-[(3R)-1-methylpyrrolidin-3-yl]pyridine-4-carboxamide

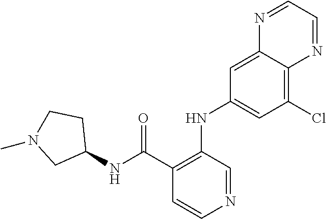 [SMILES: CN1CC[C@@H](NC(=O)c2ccncc2Nc2cc(Cl)c3nccnc3c2)C1]

The title compound is prepared according to General Procedure 22 described in Example 126, using 3-[(8-chloroquinoxalin-6-yl)amino]pyridine-4-carboxylic acid (Intermediate 104, 100 mg; 0.32 mmol; 1 eq.), HATU (184 mg; 0.48 mmol; 1.50 eq.), DIPEA (0.18 mL; 1.29 mmol; 4 eq.), (R)-1-methylpyrrolidin-3-ylamine (0.07 mL; 0.65 mmol; 2 eq.) in anhydrous DMF (2.50 mL).

Conditions: 50° C. for 2 h. Purification by FCC (30% to 40% MeOH gradient in DCM). The fractions containing the pure product are evaporated, the residue is redissolved in 3 mL of DCM and the solution is filtered on a 0.45 μm syringe filter and evaporated to dryness to yield 3-[(8-chloroquinoxalin-6-yl)amino]-N-[(3R)-1-methylpyrrolidin-3-yl]pyridine-4-carboxamide (105 mg; 0.27 mmol; 84%; yellow solid; UPLC purity: 99%).

### Example 193

### 3-{[8-(3-Methyl-1-benzothiophen-5-yl)quinoxalin-6-yl]amino}-N-[(3S)-1-methylpyrrolidin-3-yl]pyridine-4-carboxamide

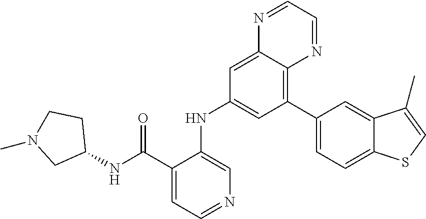 [SMILES: Cc1csc2ccc(-c3cc(Nc4cnccc4C(=O)N[C@H]4CCN(C)C4)cc4nccnc34)cc12]

The title compound is prepared according to General Procedure 17 described in Example 66, 3-[(8-chloroquinoxalin-6-yl)amino]-N-[(3S)-1-methylpyrrolidin-3-yl]pyridine-4-carboxamide (Intermediate 106, 100 mg; 0.26 mmol; 1 eq.), sodium carbonate (138 mg; 1.31 mmol; 5 eq.), 4,4,5,5-tetramethyl-2-(3-methyl-1-benzothiophen-5-yl)-1,3,2-dioxaborolane (86 mg; 0.31 mmol; 1.20 eq.), tetrakis(triphenylphosphine)palladium(0) (16 mg; 0.01 mmol; 0.05 eq.) in water (1 mL), ethanol (1 mL) and toluene (2 mL). Conditions: 100° C. overnight. Purification by FCC (30% MeOH in DCM). The fractions containing the pure product are evaporated, the residue is redissolved in 3 mL of DCM and the solution is filtered on a 0.45 μm syringe filter and evaporated to dryness to yield 3-{[8-(3-methyl-1-benzothiophen-5-yl)quinoxalin-6-yl]amino}-N-[(3S)-1-methylpyrrolidin-3-yl]pyridine-4-carboxamide (14 mg; 0.03 mmol; 11%; yellow powder; HPLC purity: 98.3%).

### Example 194

### 3-{[8-(3-Methyl-1-benzothiophen-5-yl)quinoxalin-6-yl]amino}-N-[(3R)-1-methylpyrrolidin-3-yl]pyridine-4-carboxamide

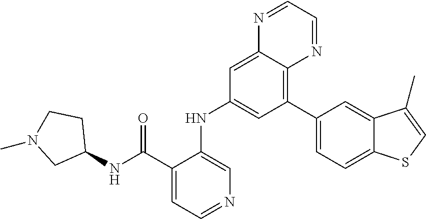 [SMILES: Cc1csc2ccc(-c3cc(Nc4cnccc4C(=O)N[C@@H]4CCN(C)C4)cc4nccnc34)cc12]

The title compound is prepared according to General Procedure 17 described in Example 66, 3-[(8-chloroquinoxalin-6-yl)amino]-N-[(3S)-1-methylpyrrolidin-3-yl]pyridine-4-carboxamide (Intermediate 105, 100 mg; 0.26 mmol; 1 eq.), sodium carbonate (138 mg; 1.31 mmol; 5 eq.), 4,4,5,5-tetramethyl-2-(3-methyl-1-benzothiophen-5-yl)-1,3,2-dioxaborolane (86 mg; 0.31 mmol; 1.20 eq.), tetrakis(triphenylphosphine)palladium(0) (16 mg; 0.01 mmol; 0.05 eq.) in water (1 mL), ethanol (1 mL) and toluene (2 mL). Conditions: 100° C. overnight. Purification by FCC (30% MeOH in DCM). The fractions containing the pure product are evaporated, the residue is redissolved in 3 mL of DCM and the solution is filtered on a 0.45 μm syringe filter and evaporated to dryness to yield 3-{[8-(3-methyl-1-benzothiophen-5-yl)quinoxalin-6-yl]amino}-N-[(3R)-1-methylpyrrolidin-3-yl]pyridine-4-carboxamide (15 mg; 0.03 mmol; 11%; yellow powder; HPLC purity: 97.4%).

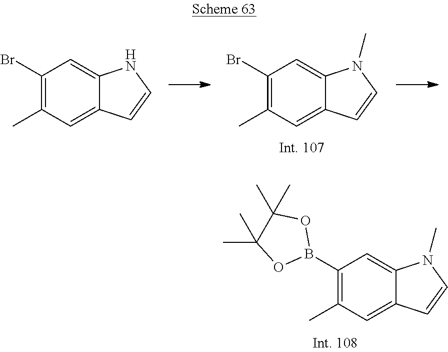 [SMILES: Cc1cc2cc[nH]c2cc1Br ; Cc1cc2ccn(C)c2cc1Br ; Cc1cc2ccn(C)c2cc1B1OC(C)(C)C(C)(C)O1]

### Intermediate 107

### 6-Bromo-1,5-dimethyl-1H-indole

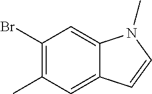

The title compound is prepared according to General Procedure 14 described for Intermediate 45, using 6-bromo-5-methyl-1H-indole (250 mg; 1.19 mmol; 1 eq.), NaH (60% in mineral oil, 95 mg; 2.38 mmol; 2 eq.), iodomethane (338 mg; 2.38 mmol; 2 eq.) in dry THF (5 mL). Conditions: 0° C. to room temperature for 15 h. The crude 6-bromo-1,5-dimethyl-1H-indole (269 mg; 0.9 mmol; 76%; UPLC purity: 75%) is used in the next step without further purification.

### Intermediate 108

### 1,5-Dimethyl-6-(tetramethyl-1,3,2-dioxaborolan-2-yl)-1H-indole

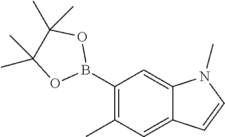 [SMILES: Cc1cc2ccn(C)c2cc1B1OC(C)(C)C(C)(C)O1]

The title compound is prepared according to General Procedure 8 described for Intermediate 14, using 6-bromo-1,5-dimethyl-1H-indole (Intermediate 107, 500 mg; 1.61 mmol; 1 eq.), bis(pinacolato)diboron (532 mg; 2.09 mmol; 1.30 eq.), potassium acetate (316 mg; 3.22 mmol; 2 eq.) and Pd(dppf)Cl₂ (12 mg; 0.02 mmol; 0.01 eq.) in dioxane (5 mL). Conditions: 100° C. overnight. Purification by FCC (0% to 5% EtOAc in hexane) gives 1,5-dimethyl-6-(tetramethyl-1,3,2-dioxaborolan-2-yl)-1H-indole (189 mg; 0.57 mmol; 35%; UPLC purity: 81%).

### Example 195

### 8-(1,5-Dimethyl-1H-indol-6-yl)-N-(4-methanesulfonylpyridin-3-yl)quinoxalin-6-amine

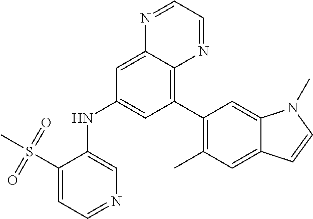 [SMILES: Cc1cc2ccn(C)c2cc1-c1cc(Nc2cnccc2S(C)(=O)=O)cc2nccnc12]

The title compound is prepared according to General Procedure 17 described in Example 66, using chloro-N-(4-methanesulfonylpyridin-3-yl)quinoxalin-6-amine (Intermediate 3B, 78 mg; 0.22 mmol; 1 eq.), sodium carbonate (119 mg; 1.12 mmol; 5 eq.), 1,5-dimethyl-6-(tetramethyl-1,3,2-dioxaborolan-2-yl)-1H-indole (Intermediate 108, 115 mg; 0.22 mmol; 1 eq.), tetrakis(triphenylphosphine)palladium(0) (14 mg; 0.01 mmol; 0.05 eq.) in water (0.50 mL), ethanol (0.50 mL) and toluene (1 mL). Conditions: 100° C. overnight. Purification by FCC (50% to 100% EtOAc gradient in hexane) yields 8-(1,5-dimethyl-1H-indol-6-yl)-N-(4-methanesulfonylpyridin-3-yl)quinoxalin-6-amine (16 mg; 0.04 mmol; 16%; yellow powder; HPLC purity: 99.6%).

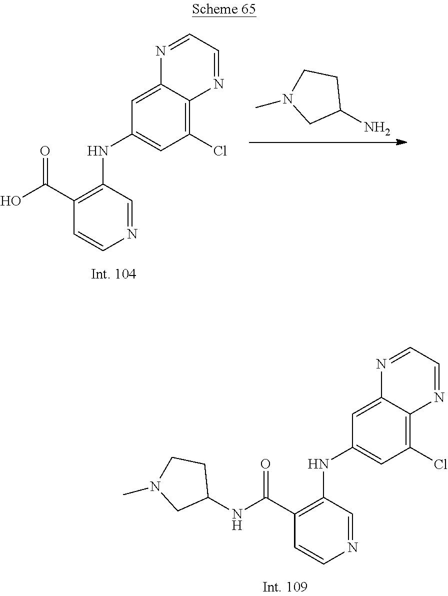

### Intermediate 109

### 3-[(8-chloroquinoxalin-6-yl)amino]-N-(1-methylpyrrolidin-3-yl)pyridine-4-carboxamide

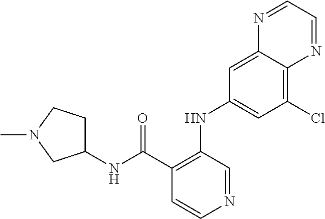 [SMILES: CN1CCC(NC(=O)c2ccncc2Nc2cc(Cl)c3nccnc3c2)C1]

The title compound is prepared according to General Procedure 22 described in Example 126, using 3-[(8-chloroquinoxalin-6-yl)amino]pyridine-4-carboxylic acid (Intermediate 104, 200 mg; 0.64 mmol; 1 eq.), HATU (368 mg; 0.96 mmol; 1.50 eq.), DIPEA (0.36 mL; 2.58 mmol; 4 eq.), 1-methylpyrrolidin-3-ylamine (0.14 mL; 1.3 mmol; 2 eq.) in anhydrous DMF (5 mL). Conditions: 50° C. for 2 h. Purification by FCC (30% to 40% MeOH gradient in DCM). The fractions containing the pure product are evaporated, the residue is redissolved in 3 mL of DCM and the solution is filtered on a 0.45 μm syringe filter and evaporated to dryness to yield 3-[(8-chloroquinoxalin-6-yl)amino]-N-(1-methylpyrrolidin-3-yl)pyridine-4-carboxamide (195 mg; 0.49 mmol; 76%; yellow solid; UPLC purity: 96%).

### Example 197

### 3-{[8-(4-fluoro-1-methyl-1H-indol-6-yl)quinoxalin-6-yl]amino}-N-(1-methylpyrrolidin-3-yl)pyridine-4-carboxamide

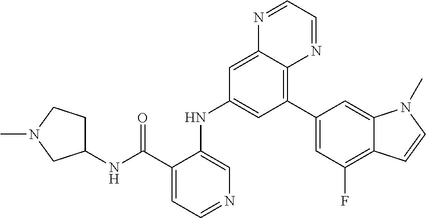 [SMILES: CN1CCC(NC(=O)c2ccncc2Nc2cc(-c3cc(F)c4ccn(C)c4c3)c3nccnc3c2)C1]

The title compound is prepared according to General Procedure 17 described in Example 66, using 3-[(8-chloroquinoxalin-6-yl)amino]-N-(1-methylpyrrolidin-3-yl)pyridine-4-carboxamide (Intermediate 109, 80 mg; 0.21 mmol; 1 eq.), sodium carbonate (111 mg; 1.04 mmol; 5 eq.), 4-fluoro-1-methyl-6-(tetramethyl-1,3,2-dioxaborolan-2-yl)-1H-indole (Intermediate 49, 69 mg; 0.25 mmol; 1.20 eq.), tetrakis(triphenylphosphine)palladium(0) (13 mg; 0.01 mmol; 0.05 eq.) in water (1 mL), ethanol (1 mL) and toluene (2 mL). Conditions: 110° C. overnight. Purification by FCC (30% to 40% MeOH in DCM). The fractions containing the pure product are evaporated, the residue is redissolved in 3 mL of DCM and the solution is filtered on a 0.45 μm syringe filter and evaporated to dryness to yield 3-{[8-(4-fluoro-1-methyl-1H-indol-6-yl)quinoxalin-6-yl]amino}-N-(1-methylpyrrolidin-3-yl)pyridine-4-carboxamide (59 mg; 0.12 mmol; 55%; yellow powder; HPLC purity: 96.9%).

### Example 198

### 3-{[8-O-Methyl-1H-indol-6-yl)quinoxalin-6-yl]oxy}pyridine-4-carboxylic acid

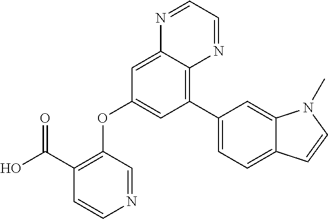 [SMILES: Cn1ccc2ccc(-c3cc(Oc4cnccc4C(=O)O)cc4nccnc34)cc21]

A pressure vessel is loaded with 8-(1-methyl-1H-indol-6-yl)quinoxalin-6-ol (Intermediate 36, 50 mg; 0.18 mmol; 1 eq.), 3-fluoroisonicotinic acid (52 mg; 0.35 mmol; 2 eq.), tBuOK (26 mg; 0.26 mmol; 1.5 eq.) and DMSO (3 mL). The vessel is sealed and the reaction mixture is heated at 150° C. for 32 h. The reaction mixture is then cooled to room temperature and partitioned between DCM and water. The aqueous layer is extracted with iPrOH/DCM (1/4) and the combined organic phases are washed with brine, dried over sodium sulfate, filtered and concentrated in vacuo. The resulting residue is purified by FCC (Puriflash CN 30 μM; 0 to 10% MeOH gradient in DCM) to afford 3-{[8-(1-methyl-1H-indol-6-yl)quinoxalin-6-yl]oxy}pyridine-4-carboxylic acid (12 mg; 0.03 mmol; 15%; yellow-brown powder; HPLC purity: 86.8%).

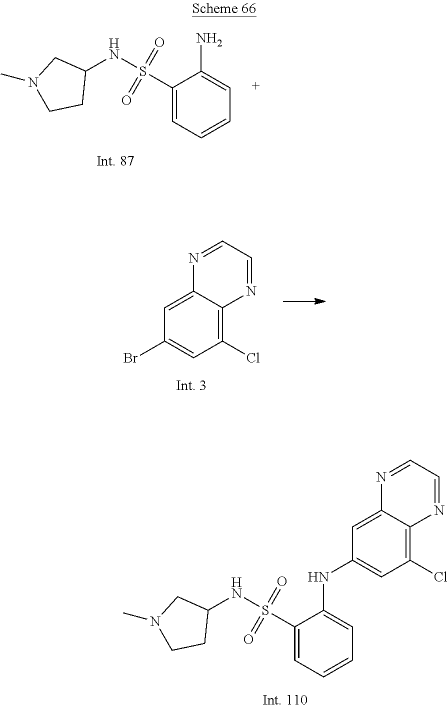 [SMILES: CN1CCC(NS(=O)(=O)c2ccccc2N)C1 ; Clc1cc(Br)cc2nccnc12 ; CN1CCC(NS(=O)(=O)c2ccccc2Nc2cc(Cl)c3nccnc3c2)C1]

### Intermediate 110

### 2-[(8-Chloroquinoxalin-6-yl)amino]-N-(1-methylpyrrolidin-3-yl)benzene-1-sulfonamide

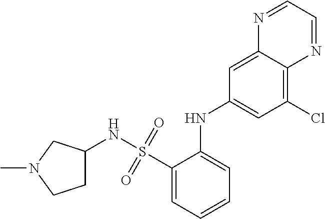 [SMILES: CN1CCC(NS(=O)(=O)c2ccccc2Nc2cc(Cl)c3nccnc3c2)C1]

The title compound is prepared according to General Procedure 1 described in Example 1, using 7-bromo-5-chloroquinoxaline (Intermediate 3, 80 mg; 0.33 mmol; 1 eq.), 2-amino-N-(1-methylpyrrolidin-3-yl)benzene-1-sulfonamide (Intermediate 87, 101 mg; 0.39 mmol; 1.2 eq.), palladium(II) acetate (7 mg; 0.03 mmol; 0.10 eq.), BINAP (41 mg; 0.07 mmol; 0.2 eq.), cesium carbonate (428 mg; 1.31 mmol; 4 eq.) and anhydrous dioxane, (5 mL). Conditions: 100° C. for 2 h. Purification by FCC (Puriflash NH2; EtOAc gradient in hexane) affords 2-[(8-chloroquinoxalin-6-yl)amino]-N-(1-methylpyrrolidin-3-yl)benzene-1-sulfonamide (80 mg; 0.15 mmol; 47%; yellow powder; UPLC purity: 80%).

### Example 199

### 2-{[8-(3-Methyl-1-benzothiophen-5-yl)quinoxalin-6-yl]amino}-N-(1-methylpyrrolidin-3-yl)benzene-1-sulfonamide

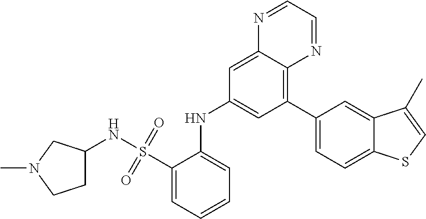 [SMILES: Cc1csc2ccc(-c3cc(Nc4ccccc4S(=O)(=O)NC4CCN(C)C4)cc4nccnc34)cc12]

A microwave vial is charged with 4,4,5,5-tetramethyl-2-(3-methyl-1-benzothiophen-5-yl)-1,3,2-dioxaborolane (47 mg; 0.17 mmol; 1.2 eq.), 2-[(8-chloroquinoxalin-6-yl)amino]-N-(1-methylpyrrolidin-3-yl)-benzene-1-sulfonamide (Intermediate 110, 60 mg; 0.14 mmol; 1 eq.), sodium carbonate (22 mg; 0.21 mmol; 1.5 eq.), dioxane (1.3 mL) and water (1.3 mL). The mixture is sparged with argon for 10 min and Pd(dppf)Cl₂ (10 mg; 0.01 mmol; 0.10 eq.) is added. The vial is sealed and the mixture is heated at 120° C. overnight. After coming back to room temperature, the reaction mixture is filtered through a pad of celite, washing the filter cake with ethyl acetate and methanol. The filtrate is concentrated in vacuo and the residue is partitioned between water and ethyl acetate. The aqueous phase is extracted with EtOAc (twice) and the combined organic layers are washed with brine, dried over Na₂SO₄, filtered and concentrated. The residue is purified by FCC (10-100% EtOAc gradient in hexane) to yield 2-{[8-(3-methyl-1-benzothiophen-5-yl)quinoxalin-6-yl]amino}-N-(1-methylpyrrolidin-3-Abenzene-1-sulfonamide (12 mg; 0.02 mmol; 15%; pale brown solid; HPLC purity: 95.1%).

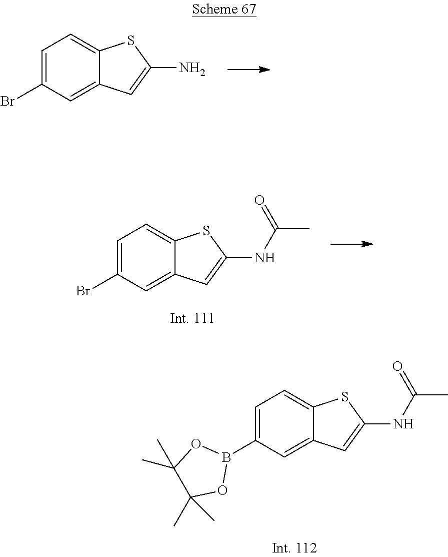 [SMILES: Nc1cc2cc(Br)ccc2s1 ; CC(=O)Nc1cc2cc(Br)ccc2s1 ; CC(=O)Nc1cc2cc(B3OC(C)(C)C(C)(C)O3)ccc2s1]

### Intermediate 111

### N-(5-Bromo-1-benzothiophen-2-yl)acetamide

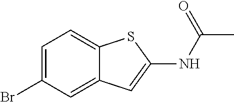

Acetyl chloride (0.03 ml; 0.39 mmol; 1.1 eq.) is added to a mixture of 5-bromo-benzothiophen-2-ylamine (80 mg; 0.35 mmol; 1 eq.), pyridine (0.08 ml; 1.05 mmol; 3 eq.), DMAP (0.43 mg; 3.5 μmol; 0.01 eq.) and anhydrous THF (5 mL) at 0° C. The resulting mixture is stirred overnight at room temperature and partitioned between water and EtOAc. The organic phase is dried over anhydrous sodium sulfate, filtered and evaporated under reduced pressure. The resulting solid is triturated in refluxing DCM for 30 minutes, filtered, washed with DCM and dried under vacuum to yield N-(5-bromo-1-benzothiophen-2-yl)acetamide (94 mg; 0.35 mmol; 99%; light beige powder, UPLC purity: 100%).

### Intermediate 112

### N-[5-(Tetramethyl-1,3,2-dioxaborolan-2-yl)-1-benzothiophen-2-yl]acetamide

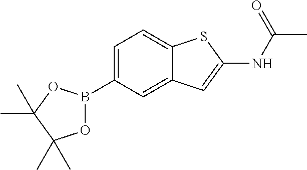 [SMILES: CC(=O)Nc1cc2cc(B3OC(C)(C)C(C)(C)O3)ccc2s1]

The title compound is prepared according to General procedure 8 using

N-(5-bromo-1-benzothiophen-2-yl)acetamide (Intermediate 111, 94 mg; 0.35 mmol; 1 eq.), bis(pinacolato)diboron (115 mg; 0.45 mmol; 1.3 eq.), potassium acetate (68 mg; 0.70 mmol; 2 eq.) and Pd(dppf)Cl₂ (25 mg; 0.03 mmol; 0.1 eq.) in dioxane (8 mL). Conditions: 100° C. overnight. Purification by FCC (0% to 10% EtOAc gradient in hexane) yields N-[5-(tetramethyl-1,3,2-dioxaborolan-2-yl)-1-benzothiophen-2-yl]acetamide (84 mg; 0.26 mmol; 76%; off-white solid; UPLC purity: 90%).

### Example 200

### N-(5-{7-[(4-Methanesulfonylpyridin-3-yl)amino]quinoxalin-5-yl}-1-benzothiophen-2-yl)acetamide

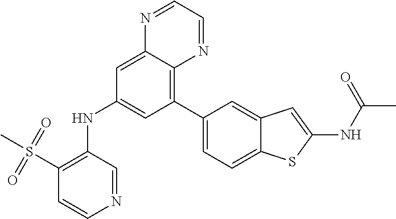 [SMILES: CC(=O)Nc1cc2cc(-c3cc(Nc4cnccc4S(C)(=O)=O)cc4nccnc34)ccc2s1]

The title compound is prepared according to General Procedure 17 described in Example 66, using 8-chloro-N-(4-methanesulfonylpyridin-3-yl)quinoxalin-6-amine (Intermediate 3B, 70 mg; 0.21 mmol; 1.00 eq.), N-[5-(tetramethyl-1,3,2-dioxaborolan-2-yl)-1-benzothiophen-2-yl]acetamide (Intermediate 112, 73 mg; 0.23 mmol; 1.1 eq.), sodium carbonate (111 mg; 1.05 mmol; 5 eq.), Tetrakis(triphenylphosphine)palladium(0) (12 mg; 0.01 mmol; 0.05 eq.) in toluene (2 mL), ethanol (1 mL) and water (1.00 ml). Conditions: 100° C., overnight. Purification FCC (0% to 10% MeOH gradient in DCM) affords N-(5-{7-[(4-methanesulfonylpyridin-3-yl)amino]quinoxalin-5-yl}-1-benzothiophen-2-yl)acetamide (45 mg; 0.08 mmol; 38%; yellow powder; HPLC purity: 90.3%).

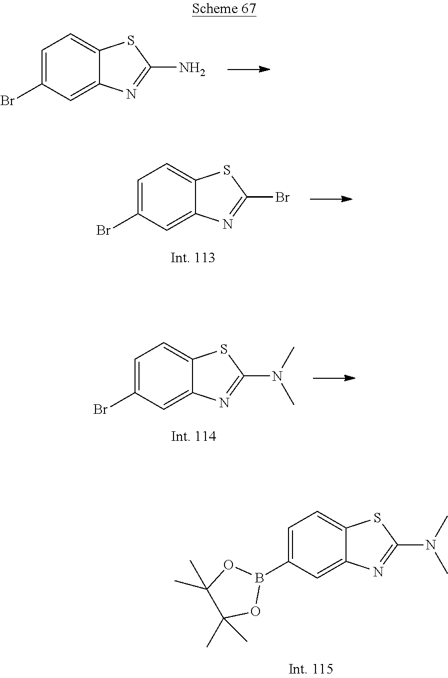

### Intermediate 113

### 2,5-Dibromo-1,3-benzothiazole

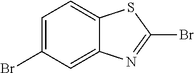 [SMILES: Brc1ccc2sc(Br)nc2c1]

To a solution of tert-butyl nitrite (0.52 ml; 4.36 mmol; 2 eq.) in anhydrous acetonitrile (15 mL), 5-bromo-benzothiazol-2-ylamine (500 mg; 2.18 mmol; 1 eq.) is added and the resulting mixture is stirred for 0.5 h at room temperature. The mixture is then warmed up to 60° C. and copper(II) bromide (731 mg; 3.27 mmol; 1.5 eq.) is added. The reaction mixture is kept with stirring for 1 hour at 60° C. and partitioned between water and ethyl acetate. The organic phase was washed with water and brine, dried over sodium sulfate and filtered through short pad of neutral alumina covered with Celite. The filtrate was evaporated to dryness to give 2,5-dibromo-1,3-benzothiazole (528 mg; 1.74 mmol; 80%; yellow solid; UPLC purity: 97%) which was used in the next step without further purification.

### Intermediate 114

### 5-Bromo-N,N-dimethyl-1,3-benzothiazol-2-amine

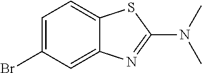

A pressure vessel is charged with 2,5-dibromo-1,3-benzothiazole (Intermediate 113, 120 mg; 0.41 mmol; 1 eq.), dimethylamine (2M in THF, 0.52 mL; 1.04 mmol; 2.5 eq.) and DMF (2 mL). The vessel is sealed and the reaction mixture is stirred at 80° C. overnight. After coming back to room temperature, the reaction mixture is evaporated to dryness and the residue is dissolved in EtOAc. The solution is washed with water and brine, dried over sodium sulfate, filtered and evaporated to dryness to give 5-bromo-N,N-dimethyl-1,3-benzothiazol-2-amine (101 mg; 0.38 mmol; 92%; UPLC purity: 96%).

### Intermediate 115

### N, N-Di methyl-5-(tetra methyl-1,3,2-dioxaborolan-2-yl)-1,3-benzothiazol-2-amine

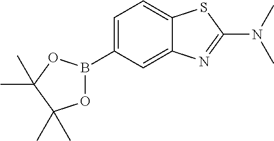

The title compound is prepared according to General procedure 8 using 5-bromo-N,N-dimethyl-1,3-benzothiazol-2-amine (Intermediate 114, 99 mg; 0.37 mmol; 1 eq.)), bis(pinacolato)diboron (142 mg; 0.56 mmol; 1.5 eq.), potassium acetate (70 mg; 0.74 mmol; 2 eq.) and Pd(dppf)Cl₂ (30 mg; 0.04 mmol; 0.1 eq.) in dioxane (1 mL). Conditions: 100° C. overnight. Purification by FCC (0 to 20% EtOAc gradient in hexane) affords N,N-dimethyl-5-(tetramethyl-1,3,2-dioxaborolan-2-yl)-1,3-benzothiazol-2-amine (134 mg; 0.36 mmol; 98%; off-white solid; UPLC purity: 83%).

### Example 201

### 8-[2-(Dimethylamino)-1,3-benzothiazol-5-yl]-N-(4-methanesulfonylpyridin-3-yl)quinoxalin-6-amine

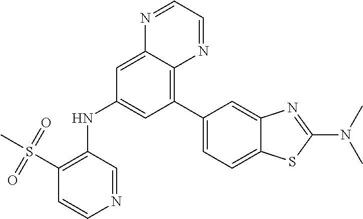

The title compound was prepared according to General Procedure 17, using chloro-N-(4-methanesulfonylpyridin-3-yl)quinoxalin-6-amine (Intermediate 3B, 50 mg; 0.15 mmol; 1 eq.), sodium carbonate (78 mg; 0.73 mmol; 5 eq.), N,N-dimethyl-5-(tetramethyl-1,3,2-dioxaborolan-2-yl)-1,3-benzothiazol-2-amine (Intermediate 115, 67 mg; 0.22 mmol; 1.5 eq.), tetrakis(triphenylphosphine)palladium(0) (9 mg; 0.01 mmol; 0.05 eq.) in water (0.50 ml), ethanol (0.50 ml) and toluene (1.00 ml). Conditions: 110° C. overnight. Purification by reversed-phase preparative HPLC (column: Gemini NX C18 5u 110A (100×30 mm), ACN gradient in water). The pure fractions are pooled, MeCN is evaporated and the resulting solution is basified with NaHCO₃. It is then extracted with EtOAc (twice) and the combined organic layers are dried (sodium sulfate) and evaporated to dryness to yield 8-[2-(dimethylamino)-1,3-benzothiazol-5-yl]-N-(4-methanesulfonylpyridin-3-yl)quinoxalin-6-amine (25 mg; 0.05 mmol; 35%; yellow powder; HPLC purity: 97.8%).

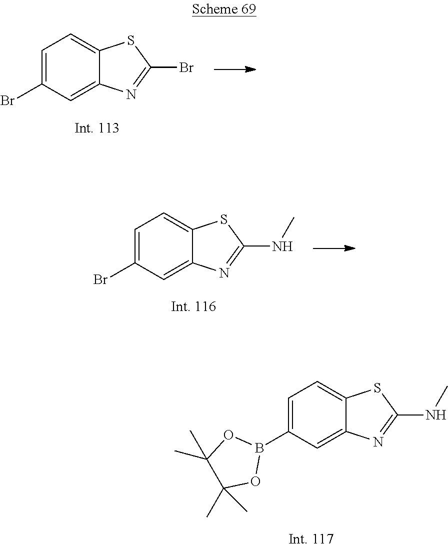

### Intermediate 116

### 5-Bromo-N-methyl-1,3-benzothiazol-2-amine

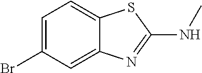

In a pressure vessel, 2,5-dibromobenzothiazole (Intermediate 113, 120 mg; 0.41 mmol; 1 eq.) was dissolved in a 2M solution of methylamine in THF (0.52 mL; 1.04 mmol; 2.5 eq.). The vessel was sealed and the reaction mixture was stirred at 60° C. overnight. After coming back to room temperature, it was diluted with EtOAc, washed with water and brine, dried over sodium sulfate, filtered and evaporated to dryness to yield 5-bromo-N-methyl-1,3-benzothiazol-2-amine (98 mg; 0.36 mmol; 87%; UPLC purity: 97%)_{.}

### Intermediate 117

### N-Methyl-5-(tetramethyl-1,3,2-dioxaborolan-2-yl)-1,3-benzothiazol-2-amine

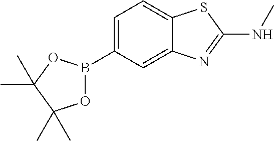

The title compound is prepared according to General procedure 8 described for intermediate 14, using 5-bromo-N-methyl-1,3-benzothiazol-2-amine (Intermediate 116, 72 mg; 0.29 mmol; 1 eq.), bis(pinacolato)diboron (110 mg; 0.43 mmol; 1.5 eq.), potassium acetate (56 mg; 0.58 mmol; 2 eq.)) and Pd(dppf)Cl₂ (23 mg; 0.03 mmol; 0.1 eq.) in dioxane (1.5 mL). Conditions: 100° C. overnight. Purification by FCC (0 to 20° A, EtOAc gradient in hexane) affords N-methyl-5-(tetramethyl-1,3,2-dioxaborolan-2-yl)-1,3-benzothiazol-2-amine (58 mg; 0.16 mmol; 56%; UPLC purity: 80%).

### Example 202

### N-(4-Methanesulfonylpyridin-3-yl)-8-[2-(methylamino)-1,3-benzothiazol-5-yl]quinoxalin-6-amine

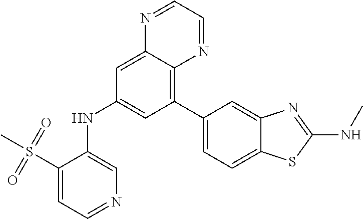

The title compound was prepared according to General Procedure 17, using chloro-N-(4-methanesulfonylpyridin-3-yl)quinoxalin-6-amine (Intermediate 3B, 46 mg; 0.13 mmol; 1 eq.), sodium carbonate (71 mg; 0.67 mmol; 5 eq.), affords N-methyl-5-(tetramethyl-1,3,2-dioxaborolan-2-yl)-1,3-benzothiazol-2-amine (Intermediate 117, 59 mg; 0.2 mmol; 1.5 eq.), Tetrakis(triphenylphosphine)palladium(0) (8 mg; 0.01 mmol; 0.05 eq.) in water (0.50 ml), ethanol (0.50 ml) and toluene (1.00 ml). Conditions: 110° C. overnight. The title compound was purified by reversed-phase preparative HPLC (column: Gemini NX C18 5u 110A (100×30 mm), ACN gradient in water, 0.1% TFA). The pure fractions are pooled, MeCN is evaporated and the resulting solution is basified with NaHCO₃. It is then extracted with EtOAc (twice) and the combined organic layers are dried (sodium sulfate) and evaporated to dryness to yield N-(4-methanesulfonylpyridin-3-yl)-8-[2-(methylamino)-1,3-benzothiazol-5-yl]quinoxalin-6-amine (16 mg; 0.03 mmol; 26%; yellow powder; HPLC purity: 96.6%).

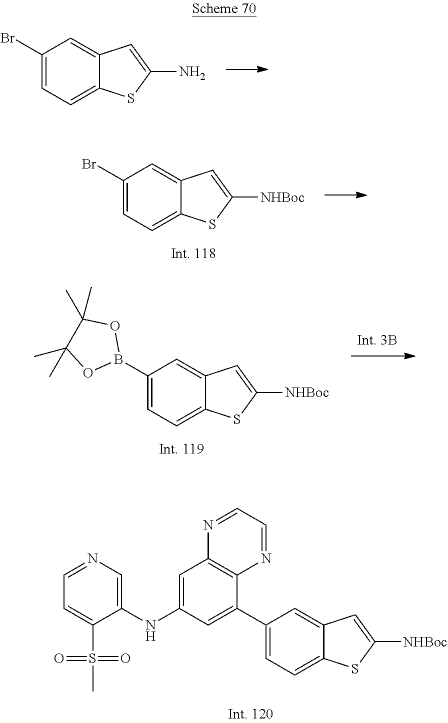 [SMILES: Nc1cc2cc(Br)ccc2s1 ; CC(C)(C)OC(=O)Nc1cc2cc(Br)ccc2s1 ; CC(C)(C)OC(=O)Nc1cc2cc(B3OC(C)(C)C(C)(C)O3)ccc2s1 ; CC(C)(C)OC(=O)Nc1cc2cc(-c3cc(Nc4cnccc4S(C)(=O)=O)cc4nccnc34)ccc2s1]

### Intermediate 118

### tert-Butyl N-(5-bromo-1-benzothiophen-2-yl)carbamate

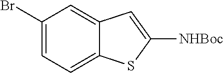 [SMILES: CC(C)(C)OC(=O)Nc1cc2cc(Br)ccc2s1]

DIPEA (0.25 ml; 1.45 mmol; 2.2 eq.), DMAP (8 mg; 0.07 mmol; 0.1 eq.) and Boc₂O (172 mg; 0.79 mmol; 1.20 eq.) were added to a solution of 5-bromo-benzothiophen-2-ylamine (150 mg; 0.66 mmol; 1 eq.) in dry THF (5 mL). The reaction was stirred at room temperature overnight and partitioned between

EtOAc and water. The aqueous phase was extracted with ethyl acetate and the combined organic layers were washed with brine, dried over anhydrous Na₂SO₄, filtered, and concentrated to dryness. The residue was purified by FCC (0% to 20% EtOAc gradient in hexane) to afford tert-butyl N-(5-bromo-1-benzothiophen-2-yl)carbamate (65 mg; 0.2 mmol; 30%; beige powder; UPLC purity: 93%).

### Intermediate 119

### tert-Butyl N-[5-(tetramethyl-1,3,2-dioxaborolan-2-yl)-1-benzothiophen-2-yl]carbamate

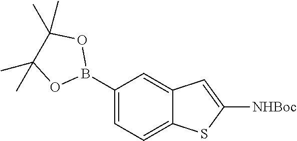 [SMILES: CC(C)(C)OC(=O)Nc1cc2cc(B3OC(C)(C)C(C)(C)O3)ccc2s1]

The title compound is prepared according to General procedure 8 described for intermediate 14, using tert-butyl N-(5-bromo-1-benzothiophen-2-yl)carbamate (Intermediate 118, 65 mg; 0.2 mmol; 1 eq.), bis(pinacolato)diboron (65 mg; 0.26 mmol; 1.3 eq.), potassium acetate (39 mg; 0.40 mmol; 2 eq.) and Pd(dppf)Cl₂ (14 mg; 0.02 mmol; 0.1 eq.) in dry dioxane (8 mL). Conditions: 100° C. overnight. Purification by FCC (0% to 10% EtOAc gradient in hexane) yields tert-butyl N-[5-(tetramethyl-1,3,2-dioxaborolan-2-yl)-1-benzothiophen-2-yl]carbamate (60 mg; 0.14 mmol; 73%; off-white crystals; UPLC purity: 90%).

### Intermediate 120

### tert-Butyl N-(5-{7-[(4-methanesulfonylpyridin-3-yl)amino]quinoxalin-5-yl}-1-benzothiophen-2-yl)carbamate

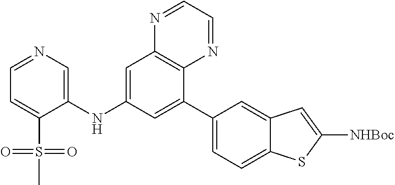 [SMILES: CC(C)(C)OC(=O)Nc1cc2cc(-c3cc(Nc4cnccc4S(C)(=O)=O)cc4nccnc34)ccc2s1]

The title compound was prepared according to General Procedure 17 described in Example 66, using chloro-N-(4-methanesulfonylpyridin-3-yl)quinoxalin-6-amine (Intermediate 3B, 50 mg; 0.15 mmol; 1 eq.), sodium carbonate (79 mg; 0.75 mmol; 5 eq.), tert-butyl N-[5-(tetramethyl-1,3,2-dioxaborolan-2-yl)-1-benzothiophen-2-yl]carbamate (Intermediate 119, 62 mg; 0.16 mmol; 1.1 eq.), Tetrakis(triphenylphosphine)palladium(0) (9 mg; 0.01 mmol; 0.05 eq.) in water (1 mL), ethanol (1 mL) and toluene (2 mL). Conditions: 110° C. overnight. Purification by FCC (0% to 10% MeOH gradient in DCM) yields tert-butyl N-(5-{7-[(4-methanesulfonylpyridin-3-yl)amino]quinoxalin-5-yl}-1-benzothiophen-2-yl)carbamate (60 mg; 0.11 mmol; 73%; yellow powder; UPLC purity: 92%).

### Example 203

### 8-(2-Amino-1-benzothiophen-5-yl)-N-(4-methanesulfonylpyridin-3-yl)quinoxalin-6-amine

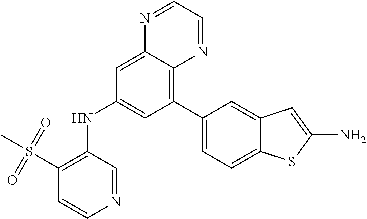 [SMILES: CS(=O)(=O)c1ccncc1Nc1cc(-c2ccc3sc(N)cc3c2)c2nccnc2c1]

TFA (2.00 mL) was added to a solution of tert-butyl N-(5-{7-[(4-methanesulfonylpyridin-3-yl)amino]quinoxalin-5-yl}-1-benzothiophen-2-yl)carbamate (Intermediate 120, 60 mg; 0.11 mmol; 1 eq.) and the mixture was stirred at room temperature for 3 h. It was then quenched with aqueous 1N NaOH, and aqueous sat. NaHCO₃ and extracted with n-butanol. The organic layer was dried over sodium sulfate, filtered and evaporated to a residue which was purified by FCC (0% to 10% MeOH gradient in DCM) to yield 8-(2-amino-1-benzothiophen-5-yl)-N-(4-methanesulfonylpyridin-3-yl)quinoxalin-6-amine (20 mg; 0.04 mmol; 37%; dark yellow powder; HPLC purity: 89.8%).

### Example 204

### N-(5-Bromopyrimidin-4-yl)-8-(1-methyl-1H-indol-6-yl)quinoxalin-6-amine

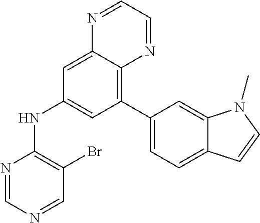 [SMILES: Cn1ccc2ccc(-c3cc(Nc4ncncc4Br)cc4nccnc34)cc21]

The title compound was prepared according to General Procedure 1 described in Example 1, using 8-(1-methyl-1H-indol-6-yl)quinoxalin-6-amine (Intermediate 4, 419 mg; 1.45 mmol; 1 eq.), 5-bromopyrimidine-4-carbonitrile (440 mg; 2.39 mmol; 1.65 eq.), cesium carbonate (1.9 g; 5.8 mmol; 4.00 eq.), BINAP (180 mg; 0.29 mmol; 0.2 eq), palladium(II) acetate (33 mg; 0.14 mmol; 0.10 eq.) in dry NMP (10 mL). Conditions: 200° C. for 2 hours. Purification by FCC (50% to 100% EtOAc gradient in hexane) followed by purified by reversed-phase preparative HPLC (column: Gemini NX C18 5u 110A (100×30 mm), ACN gradient in water, 0.1% TFA). The pure fractions are pooled, MeCN is evaporated and the resulting solution is basified with NaHCO₃. It is then extracted with EtOAc (twice) and the combined organic layers are dried (sodium sulfate) and evaporated to dryness to yield N-(5-bromopyrimidin-4-yl)-8-(1-methyl-1H-indol-6-yl)quinoxalin-6-amine (138 mg; 0.30 mmol; 21%; yellow powder; HPLC purity: 95.0%).

### Example 205

### 3-{[8-(3-Methyl-1-benzothiophen-5-yl)quinoxalin-6-yl]amino}pyridine-4-carboxamide

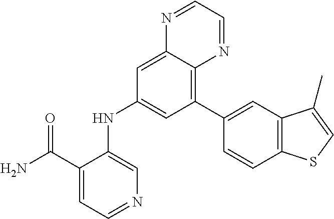 [SMILES: Cc1csc2ccc(-c3cc(Nc4cnccc4C(N)=O)cc4nccnc34)cc12]

The title compound was prepared according to General Procedure 13 described in Example 52, using 3-{[8-(3-methyl-1-benzothiophen-5-yl)quinoxalin-6-yl]amino}pyridine-4-carboxylic acid (Intermediate 72, 100 mg; 0.22 mmol; 1 eq.), ammonium chloride (77 mg; 1.37 mmol; 6.3 eq.), EDC x HCl (68 mg; 0.35 mmol; 1.6 eq.), HOAt (52 mg; 0.38 mmol; 1.7 eq.), DIPEA (0.09 mL; 0.50 mmol; 2.3 eq.) and dry DMF (5 mL). Conditions: overnight at rt. Purification by FCC (0% to 4% MeOH gradient in DCM) yields 3-{[8-(3-methyl-1-benzothiophen-5-yl)quinoxalin-6-yl]amino}pyridine-4-carboxamide (15 mg; 0.03 mmol; 16%; yellow powder; HPLC purity: 96.0%).

### Example 206

### N-(1-Acetylazetidin-3-yl)-3-{[8-(3-methyl-1-benzothiophen-5-yl)quinoxalin-6-yl]amino}pyridine-4-carboxamide

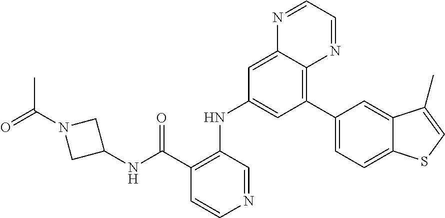 [SMILES: CC(=O)N1CC(NC(=O)c2ccncc2Nc2cc(-c3ccc4scc(C)c4c3)c3nccnc3c2)C1]

1-(3-Aminoazetidin-1-yl)ethan-1-one hydrochloride (46 mg; 0.31 mmol; 3 eq.), DMTMM (113 mg; 0.41 mmol; 4 eq.) and DIPEA (0.09 ml; 0.51 mmol; 5 eq.) were sequentially added at rt to a solution of 3-{[8-(3-methyl-1-benzothiophen-5-yl)quinoxalin-6-yl]amino}pyridine-4-carboxylic acid (Intermediate 72, 70 mg; 0.10 mmol; 1 eq.) in anhydrous DMF (1 mL) and the reaction mixture was stirred overnight at rt under argon. It was then diluted with EtOAc and the solution was washed with water and brine. The organic layer was dried over sodium sulfate, filtered and evaporated to a residue which was purified by FCC (0% to 5% MeOH gradient in DCM) to yield N-(1-acetylazetidin-3-yl)-3-{[8-(3-methyl-1-benzothiophen-5-yl)quinoxalin-6-yl]amino}pyridine-4-carboxamide (8 mg; 0.01 mmol; 14%; yellow crystals; HPLC purity: 93.8%).

### Example 207

### 3-{[8-(1-Methyl-1H-indol-6-yl)quinoxalin-6-yl]amino}-N-(pyridin-3-yl)pyridine-4-carboxamide

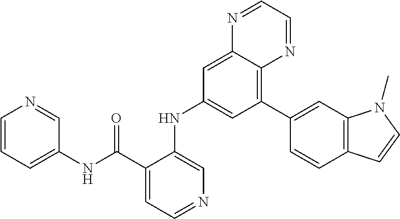 [SMILES: Cn1ccc2ccc(-c3cc(Nc4cnccc4C(=O)Nc4cccnc4)cc4nccnc34)cc21]

The title compound is prepared according to General Procedure 22 described in example 126, using 3-{[8-(1-methyl-1H-indol-6-yl)quinoxalin-6-yl]amino}pyridine-4-carboxylic acid (Intermediate 42, 50 mg; 0.13 mmol; 1 eq.), HATU (72 mg; 0.19 mmol; 1.5 eq.), DIPEA (0.14 mL; 1.01 mmol; 8 eq.), pyridin-3-ylamine (12 mg; 0.13 mmol; 1 eq.) in anhydrous DMF (5 mL). Conditions: 50° C. for 12 h. Purification by FCC (0 to 20% MeOH gradient in DCM) followed by reversed-phase preparative HPLC (column: Gemini NX C18 5u 110A (100×30 mm), ACN gradient in water, 0.1% TFA). The pure fractions are pooled, MeCN is evaporated and the resulting solution is basified with NaHCO₃. It is then extracted with DCM (twice) and the combined organic layers are dried (sodium sulfate) and evaporated to dryness to yield 3-{[8-(1-methyl-1H-indol-6-yl)quinoxalin-6-yl]amino}-N-(pyridin-3-yl)pyridine-4-carboxamide (10 mg; 0.02 mmol; 15%; yellow powder; HPLC purity: 89.9%).

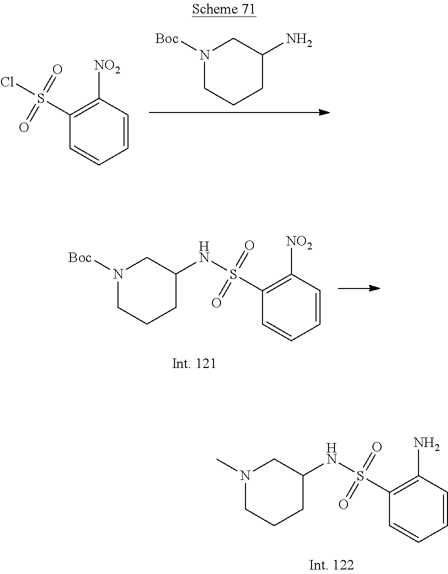 [SMILES: O=[N+]([O-])c1ccccc1S(=O)(=O)Cl ; CC(C)(C)OC(=O)N1CCCC(N)C1 ; CC(C)(C)OC(=O)N1CCCC(NS(=O)(=O)c2ccccc2[N+](=O)[O-])C1 ; CN1CCCC(NS(=O)(=O)c2ccccc2N)C1]

### Intermediate 121

### tert-Butyl 3-(2-nitrobenzenesulfonamido)piperidine-1-carboxylate

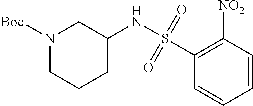 [SMILES: CC(C)(C)OC(=O)N1CCCC(NS(=O)(=O)c2ccccc2[N+](=O)[O-])C1]

2-Nitrobenzenesulfonyl chloride (200 mg; 0.90 mmol; 1 eq.), 3-aminopiperidine-1-carboxylic acid tert-butyl ester hydrochloride (320 mg; 1.35 mmol; 1.5 eq.), DIPEA (0.47 mL; 2.71 mmol; 3 eq.), THF anhydrous (10 mL) are stirred at room temperature for 16 h. The reaction mixture is evaporated under reduced pressure, dissolved in EtOAc (50 mL), washed with water (3×20 mL) and brine (2×10 mL). The organic layer is dried over Na₂SO₄, filtered, and evaporated to dryness. The crude tert-butyl 3-(2-nitrobenzenesulfonamido)piperidine-1-carboxylate (354 mg; 0.90 mmol; 99%; yellow oil; UPLC purity: 98%) is used in the next step without further purification.

### Intermediate 122

### 2-Amino-N-(1-methylpiperidin-3-yl)benzene-1-sulfonamide

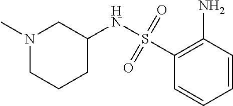 [SMILES: CN1CCCC(NS(=O)(=O)c2ccccc2N)C1]

Lithium aluminum hydride (1.0 M in THF, 1.74 mL; 1.74 mmol; 2 eq.) is added to a solution tert-butyl 3-(2-nitrobenzenesulfonamido)-piperidine-1-carboxylate (Intermediate 121, 350 mg; 0.87 mmol; 1.00 eq.) in anhydrous THF (12 mL) at 0° C. The reaction is stirred for 20 hours while gradually warming to room temperature and at 60° C. for another 6 extra hours. The reaction is then quenched with 1.0 M sodium hydroxide and partitioned between water and EtOAc. The organic layer is washed with water and the combined aqueous layers are extracted with DCM:isopropanol (4:1). The combined organic layers are dried over Na₂SO₄, filtered and concentrated to dryness. The crude 2-amino-N-(1-methylpiperidin-3-yl)benzene-1-sulfonamide (135 mg; 0.50 mmol; 57%; UPLC purity: 96%) is used in the next step without further purification.

### Example 208

### 2-{[8-(1-Methyl-1H-indol-6-yl)quinoxalin-6-yl]amino}-N-(1-methylpiperidin-3-yl)benzene-1-sulfonamide

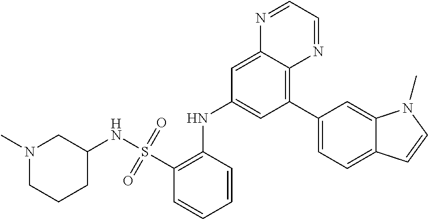 [SMILES: CN1CCCC(NS(=O)(=O)c2ccccc2Nc2cc(-c3ccc4ccn(C)c4c3)c3nccnc3c2)C1]

The title compound is prepared according to General Procedure 1 described in Example 1, using 7-chloro-5-(1-methyl-1H-indol-6-yl)quinoxaline (Intermediate 2B, 60 mg; 0.20 mmol; 1 eq.), 2-amino-N-(1-methylpiperidin-3-yl)benzene-1-sulfonamide (Intermediate 122, 106 mg; 0.30 mmol; 1.5 eq.), cesium carbonate (325 mg; 1.00 mmol; 5 eq.), BINAP (12 mg; 0.02 mmol; 0.1 eq.), palladium(II) acetate (5 mg; 0.02 mmol; 0.1 eq.) and anhydrous dioxane (2 mL). Conditions: 150° C. for 1.5 hours. Purification by FCC (0% to 10% MeOH gradient in DCM) affords 2-{[8-(1-methyl-1H-indol-6-yl)quinoxalin-6-yl]amino}-N-(1-methylpiperidin-3-yl)benzene-1-sulfonamide (34 mg; 0.06 mmol; 32%; yellow powder; HPLC purity: 97.7%).

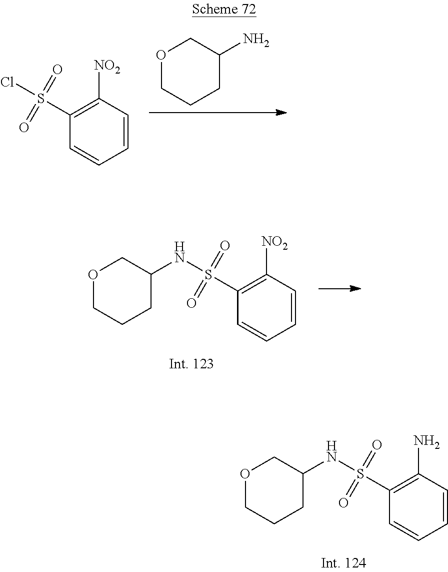

### Intermediate 123

### 2-Nitro-N-(oxan-3-yl)benzene-1-sulfonamide

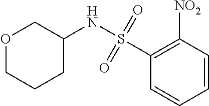 [SMILES: O=[N+]([O-])c1ccccc1S(=O)(=O)NC1CCCOC1]

2-Nitrobenzenesulfonyl chloride (200 mg; 0.90 mmol; 1.00 eq.), tetrahydropyran-3-ylamine (183 mg; 1.80 mmol; 2 eq.), DIPEA (0.47 mL; 2.71 mmol; 3 eq.), THF anhydrous (10 mL) are stirred at room temperature for 16 h. The reaction mixture is evaporated under reduced pressure and the residue is dissolved in EtOAc (50 mL). The solution is washed with water and brine, and the organic layer is dried over Na₂SO₄, filtered, and evaporated to dryness. The crude 2-nitro-N-(oxan-3-yl)benzene-1-sulfonamide (220 mg; 0.75 mmol; 83%; yellow oil; UPLC purity: 97%) is used in next step without further purification.

### Intermediate 124

### 2-Amino-N-(oxan-3-yl)benzene-1-sulfonamide

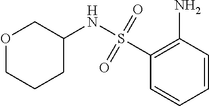 [SMILES: Nc1ccccc1S(=O)(=O)NC1CCCOC1]

A round-bottomed flask is charged with 2-nitro-N-(oxan-3-yl)-benzene-1-sulfonamide (Intermediate 123, 0.22 g; 0.71 mmol; 1 eq.), ethanol (2 mL), water (1 mL), acetic acid (2 mL; 34.94 mmol; 50 eq.) and iron (158 mg; 2.83 mmol; 4 eq.) and the reaction mixture is sonicated at 30° C. for 1 hour. It is then filtered over celite and concentrated under vacuum. The residue is partitioned between water and DCM:isopropanol (4:1) and the aqueous layer is extracted twice with DCM:isopropanol (4:1). The combined organic layers are dried over Na₂SO₄, filtered and concentrated to dryness. The crude 2-amino-N-(oxan-3-yl)benzene-1-sulfonamide (187 mg; 0.69 mmol; 98%; UPLC purity: 99%) is used in the next step without further purification.

### Example 209

### 2-{[8-(1-Methyl-1H-indol-6-yl)quinoxalin-6-yl]amino}-N-(oxan-3-yl)benzene-1-sulfonamide

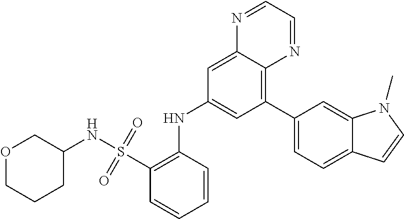 [SMILES: Cn1ccc2ccc(-c3cc(Nc4ccccc4S(=O)(=O)NC4CCCOC4)cc4nccnc34)cc21]

The title compound is prepared according to General Procedure 1 described in Example 1, using 7-chloro-5-(1-methyl-1H-indol-6-yl)quinoxaline (Intermediate 2B, 90 mg; 0.30 mmol; 1 eq.), 2-amino-N-(oxan-3-yl)benzene-1-sulfonamide (Intermediate 124, 186 mg; 0.45 mmol; 1.5 eq.), cesium carbonate (488 mg; 1.50 mmol; 5 eq.), BINAP (19 mg; 0.03 mmol; 0.1 eq.), palladium(II) acetate (7 mg; 0.03 mmol; 0.1 eq.) and anhydrous dioxane (2 mL). Conditions: 150° C. for 1.5 hours. Purification by FCC (Puriflash NH2, 0% to 100% EtOAc gradient in hexane) followed by reversed-phase preparative HPLC (column: Gemini NX C18 5u 110A (100×30 mm), ACN gradient in water, 0.1% TFA). The pure fractions are pooled, MeCN is evaporated and the resulting solution is basified with aq. 1M NaOH. It is then extracted with DCM (twice) and the combined organic layers are dried (sodium sulfate) and evaporated to dryness to yield 2-{[8-(1-methyl-1H-indol-6-yl)quinoxalin-6-yl]amino}-N-(oxan-3-yl)benzene-1-sulfonamide (8 mg; 0.01 mmol; 5%; yellow powder; HPLC purity: 86.6%).

### Example 210

### N-(4-Methanesulfonylpyridin-3-yl)-8-[3-(methylsulfanyl)phenyl]quinoxalin-6-amine

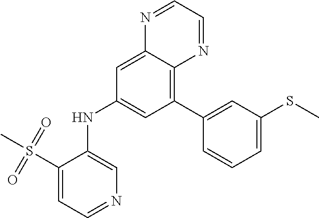

The title compound is prepared according to General Procedure 17 described in Example 66, using chloro-N-(4-methanesulfonylpyridin-3-yl)quinoxalin-6-amine (Intermediate 3B, 75 mg; 0.22 mmol; 1.00 eq.), sodium carbonate (115 mg; 1.09 mmol; 5 eq.), 4,4,5,5-tetramethyl-2-(3-methylsulfanylphenyl)-[1,3,2]dioxaborolane (80 mg; 0.24 mmol; 1.1 eq.), tetrakis(triphenylphosphine)palladium(0) (13 mg; 0.01 mmol; 0.05 eq.) in water (0.5 mL), ethanol (0.5 mL) and toluene (1 mL). Conditions: 110° C. overnight. Purification by FCC (0% to 5% MeOH gradient in DCM) yields N-(4-methanesulfonylpyridin-3-yl)-8-[3-(methylsulfanyl)phenyl]quinoxalin-6-amine (64 mg; 0.15 mmol; 69%; yellow powder; HPLC purity: 98.5%).

### Example 211

### 8-(4-Bromo-3-fluorophenyl)-N-(4-methanesulfonylpyridin-3-yl)quinoxalin-6-amine

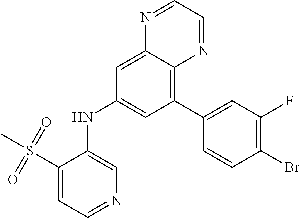 [SMILES: CS(=O)(=O)c1ccncc1Nc1cc(-c2ccc(Br)c(F)c2)c2nccnc2c1]

Anhydrous copper(II) bromide (134 mg; 0.60 mmol; 1.25 eq.), tert-butyl nitrite (100 μl; 0.84 mmol; 1.75 eq.), and anhydrous acetonitrile (4 mL) are added to a 10-mL round-bottom flask under argon and the solution is heated to 65° C. with stirring under argon. In a separate 25-mL round-bottom flask, 8-(4-amino-3-fluorophenyl)-N-(4-methanesulfonylpyridin-3-yl)quinoxalin-6-amine (Example 186, 200 mg; 0.48 mmol; 1 eq.) is suspended in anhydrous acetonitrile (4 mL) and heated to 65° C. for 10 min. The solution from the first flask is then added dropwise over 5 min to the second solution using syringe techniques. The reaction mixture is left with stirring at 65° C. under argon for 1 h. After coming back to room temperature, the reaction is quenched with water and extracted with EtOAc. The organic phase is dried over sodium sulfate, filtered and concentrated to dryness. The residue is dissolved in DCM and the solution is filtered over a short pad of neutral allumina, eluting with EtOAc. The filtrate is evaporated to dryness and the resulting residue is purified by reversed-phase preparative HPLC (column: Gemini NX C18 5u 110A (100×30 mm), ACN gradient in water, 0.1% TFA). The pure fractions are pooled, MeCN is evaporated and the resulting solution is basified with NaHCO₃. It is then extracted with DCM (twice) and the combined organic layers are dried (sodium sulfate) and evaporated to dryness to yield give 8-(4-bromo-3-fluorophenyl)-N-(4-methanesulfonylpyridin-3-yl)quinoxalin-6-amine (19 mg; 0.04 mmol; 8%; yellow powder; HPLC purity: 97.4%).

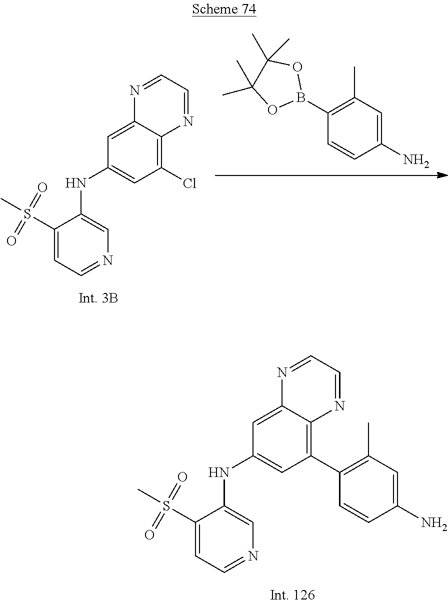

### Intermediate 126

### 8-(4-Amino-2-methylphenyl)-N-(4-methanesulfonylpyridin-3-yl)-quinoxalin-6-amine

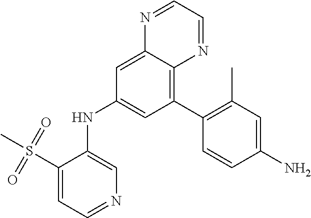 [SMILES: Cc1cc(N)ccc1-c1cc(Nc2cnccc2S(C)(=O)=O)cc2nccnc12]

The title compound is prepared according to General Procedure 17 described in Example 66, using chloro-N-(4-methanesulfonylpyridin-3-yl)quinoxalin-6-amine (Intermediate 3B, 310 mg; 0.89 mmol; 1 eq.), sodium carbonate (471 mg; 4.44 mmol; 5 eq.), 3-methyl-4-(4,4,5,5-tetramethyl-[1,3,2]dioxaborolan-2-yl)-phenylamine (249 mg; 1.07 mmol; 1.2 eq.), tetrakis(triphenylphosphine)palladium(0) (54 mg; 0.04 mmol; 0.05 eq.) in water (3 mL), ethanol (3 mL) and toluene (6 mL). Conditions: 100° C. overnight. Purification by FCC (50% to 100% EtOAc gradient in hexane) yields 8-(4-amino-2-methylphenyl)-N-(4-methanesulfonyl-pyridin-3-yl)quinoxalin-6-amine (243 mg; 0.49 mmol; 55%; yellow solid; UPLC purity: 81%).

### Example 212

### 8-(4-Bromo-2-methylphenyl)-N-(4-methanesulfonylpyridin-3-yl)quinoxalin-6-amine

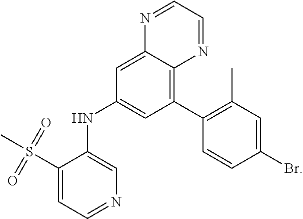 [SMILES: Cc1cc(Br)ccc1-c1cc(Nc2cnccc2S(C)(=O)=O)cc2nccnc12]

Anhydrous copper(II) bromide (136 mg; 0.61 mmol; 1.25 eq.), tert-butyl nitrite (101 μl; 0.85 mmol; 1.75 eq.), and anhydrous acetonitrile (4 mL) are added to a 10-mL round-bottom flask under argon and the solution is heated to 65° C. with stirring under argon. In a separate 25-mL round-bottom flask, 8-(4-amino-2-methylphenyl)-N-(4-methanesulfonyl-pyridin-3-yl)quinoxalin-6-amine (Intermediate 126, 243 mg; 0.49 mmol; 1 eq.) is suspended in anhydrous acetonitrile (4 mL) and heated to 65° C. for 10 min. The solution from the first flask is then added dropwise over 5 min to the second solution using syringe techniques. The reaction mixture is left with stirring at 65° C. under argon for 1 h. After coming back to room temperature, the reaction is quenched with water and extracted with EtOAc. The organic phase is dried over sodium sulfate, filtered and concentrated to dryness. The residue is dissolved in DCM and the solution is filtered over a short pad of neutral allumina, eluting with EtOAc. The filtrate is evaporated to dryness and the resulting residue is purified by reversed-phase preparative HPLC (column: Gemini NX C18 5u 110A (100×30 mm), ACN gradient in water, 0.1% TFA). The pure fractions are pooled, MeCN is evaporated and the resulting solution is basified with NaHCO₃. It is then extracted with DCM (twice) and the combined organic layers are dried (sodium sulfate) and evaporated to dryness to yield give 8-(4-bromo-2-methylphenyl)-N-(4-methanesulfonylpyridin-3-yl)quinoxalin-6-amine (30 mg; 0.06 mmol; 13%; yellow powder; HPLC purity: 87.8%).

### Example 213

### N-(4-Methanesulfonylpyridin-3-yl)-8-[4-(pentafluoro-λ⁶-sulfanyl)phenyl]quinoxalin-6-amine

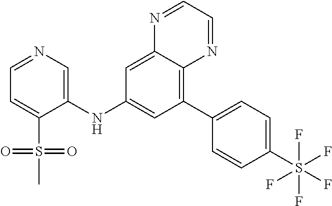 [SMILES: CS(=O)(=O)c1ccncc1Nc1cc(-c2ccc(S(F)(F)(F)(F)F)cc2)c2nccnc2c1]

A pressure vessel is charged with 8-chloro-N-(4-methanesulfonylpyridin-3-yl)-quinoxalin-6-amine (Intermediate 3B, 150 mg; 0.45 mmol; 1 eq.), [4-(4,4,5,5-tetramethyl-1,3,2-dioxaborolan-2-yl)phenyl]sulfur pentafluoride (444 mg; 1.34 mmol; 3 eq.), sodium carbonate (142 mg; 1.34 mmol; 3 eq.) and toluene (3 mL). The reaction mixture is sparged with argon for 15 min and Pd₂(dba)₃ (41 mg; 0.04 mmol; 0.1 eq.) is added followed by Xantphos (52 mg; 0.09 mmol; 0.2 eq.). The vessel is sealed and the reaction mixture is stirred at 100° C. for 4 h. After coming back to room temperature, the volatiles are evaporated in vacuo and the residue is partitioned between EtOAc and water. The aqueous phase is extracted with EtOAc and the combined organic phases are washed with brine, dried (sodium sulfate) and filtered. The filtrate is concentrated in vacuo to a volume of −10 mL and filtered on pad of allumina (5 mm) covered with celite (2 cm), eluting with EtOAc. The filtrate is concentrated in vacuo to a residue which is purified by FCC (50% to 100% EtOAc gradient in hexane) followed by reversed-phase preparative HPLC (column: Gemini NX C18 5u 110A (100×30 mm), ACN gradient in water, 0.1% TFA). The pure fractions are pooled, MeCN is evaporated and the resulting solution is basified with NaHCO₃. It is then extracted with DCM (twice) and the combined organic layers are dried (sodium sulfate) and evaporated to dryness to yield N-(4-methanesulfonylpyridin-3-yl)-8-[4-(pentafluoro-λ⁶-sulfanyl)phenyl]quinoxalin-6-amine (29 mg; 0.06 mmol; 13%; yellow powder; HPLC purity: 98.9%).

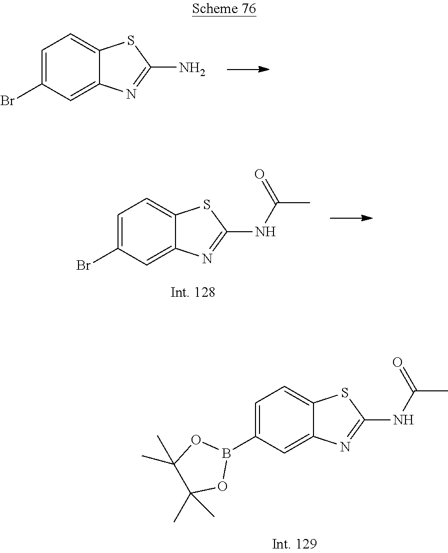 [SMILES: Nc1nc2cc(Br)ccc2s1 ; CC(=O)Nc1nc2cc(Br)ccc2s1 ; CC(=O)Nc1nc2cc(B3OC(C)(C)C(C)(C)O3)ccc2s1]

### Intermediate 128

### N-(5-Bromo-1,3-benzothiazol-2-yl)acetamide

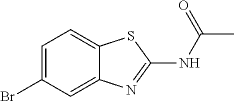

Acetyl chloride (1.7 ml; 24.01 mmol; 1.1 eq.) is added at 0° C. to a solution of 5-bromobenzothiazol-2-ylamine (5 g; 21.82 mmol; 1 eq.) and DMAP (21 mg; 0.17 mmol; 0.01 eq.) in anhydrous pyridine (5.3 mL; 65.47 mmol; 3 eq.) and anhydrous THF (50 mL). The resulting mixture is stirred overnight under, being allowed to slowly come back to room temperature. It is then poured onto ice/water and the resulting mixture is extracted with EtOAc. The organic phase is dried over sodium sulfate, filtered and evaporated under reduced pressure. The resulting solid is recrystallized from DCM to give N-(5-bromo-1,3-benzothiazol-2-yl)acetamide (4.43 g; 16.34 mmol; 75%; light beige powder; UPLC purity: 100%).

### Intermediate 129

### N-[5-(Tetramethyl-1,3,2-dioxaborolan-2-yl)-1,3-benzothiazol-2-yl]acetamide

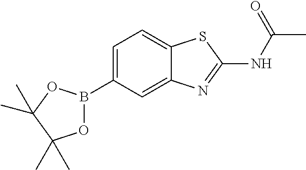 [SMILES: CC(=O)Nc1nc2cc(B3OC(C)(C)C(C)(C)O3)ccc2s1]

The title compound is prepared according to General procedure 8 described for intermediate 14, using N-(5-bromo-1,3-benzothiazol-2-yl)acetamide (Intermediate 128, 2 g; 7.38 mmol; 1 eq.), bis(pinacolato)diboron (2.4 g; 9.59 mmol; 1.3 eq.), potassium acetate (1.45 g; 14.75 mmol; 2 eq.) and Pd(dppf)Cl₂ (602 mg; 0.74 mmol; 0.10 eq.) in dioxane (20 mL). Conditions: 100° C. overnight. Purification by FCC (0% to 50% EtOAc gradient in DCM). After evaporation of the relevant fractions, the residue is triturated in hexane, filtered and dried under vacuum to yield N-[5-(tetramethyl-1,3,2-dioxaborolan-2-yl)-1,3-benzothiazol-2-yl]acetamide (1.38 g; 3.97 mmol; 54%; beige powder; UPLC purity: 92%).

### Example 214

### 3-{[8-(2-Amino-1,3-benzothiazol-5-yl)quinoxalin-6-yl]amino}-N-(1-methylpyrrolidin-3-yl)pyridine-4-carboxamide

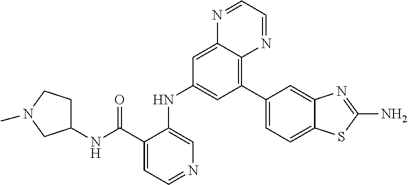 [SMILES: CN1CCC(NC(=O)c2ccncc2Nc2cc(-c3ccc4sc(N)nc4c3)c3nccnc3c2)C1]

The title compound is prepared according to General Procedure 17 described in Example 66, using 3-[(8-chloroquinoxalin-6-yl)-amino]N-(1-methylpyrrolidin-3-yl)pyridine-4-carboxamide (Intermediate 109, 100 mg; 0.26 mmol; 1 eq.), sodium carbonate (138 mg; 1.31 mmol; 5 eq.), N-[5-(tetramethyl-1,3,2-dioxaborolan-2-yl)-1,3-benzothiazol-2-yl]acetamide (Intermediate 129, 100 mg; 0.31 mmol; 1.2 eq.), tetrakis-(triphenylphosphine)palladium(0) (16 mg; 0.01 mmol; 0.05 eq.) in water (1 mL), ethanol (1 mL) and toluene (2 mL). Conditions: 110° C. overnight. Purification by reversed-phase preparative HPLC (column: Gemini NX C18 5U 110A (100×30 mm), ACN gradient in water, 0.1% TFA). The pure fractions are pooled, MeCN is evaporated and the resulting solution is basified with NaHCO₃. It is then extracted with DCM (twice) and the combined organic layers are dried (sodium sulfate) and evaporated to dryness to yield 3-{[8-(2-amino-1,3-benzothiazol-5-yl)quinoxalin-6-yl]amino}-N-(1-methylpyrrolidin-3-yl)pyridine-4-carboxamide (22 mg; 0.04 mmol; 17%; yellow powder; HPLC purity: 99.4%).

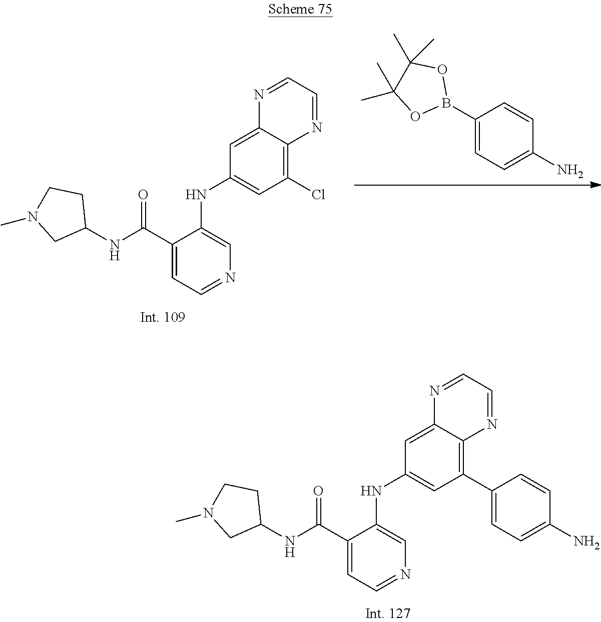

### Intermediate 127

### 3-{[8-(4-Aminophenyl)quinoxalin-6-yl]amino}-N-(1-methylpyrrolidin-3-yl)pyridine-4-carboxamide

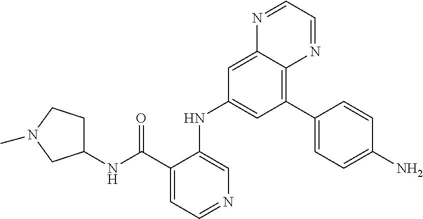 [SMILES: CN1CCC(NC(=O)c2ccncc2Nc2cc(-c3ccc(N)cc3)c3nccnc3c2)C1]

The title compound is prepared according to General Procedure 17 described in Example 66, using 3-[(8-chloroquinoxalin-6-yl)amino]-N-(1-methyl-pyrrolidin-3-yl)pyridine-4-carboxamide (Intermediate 109, 200 mg; 0.52 mmol; 1 eq.), sodium carbonate (277 mg; 2.61 mmol; 5 eq.), 4-(4,4,5,5-tetramethyl-[1,3,2]dioxaborolan-2-yl)-phenylamine (137 mg; 0.63 mmol; 1.2 eq.), tetrakis(triphenylphosphine)palladium(0) (32 mg; 0.03 mmol; 0.05 eq.) in water (2 mL), ethanol (2 mL) and toluene (4 mL). Conditions: 110° C. overnight. Purification by FCC (0% to 10% MeOH gradient in DCM) yields 3-{[8-(4-aminophenyl)quinoxalin-6-yl]-amino}-N-(1-methylpyrrolidin-3-yl)pyridine-4-carboxamide (160 mg; 0.36 mmol; 69%, UPLC purity: 99%).

### Example 215

### 3-{[8-(4-Bromophenyl)quinoxalin-6-yl]amino}-N-(1-methylpyrrolidin-3-yl)pyridine-4-carboxamide

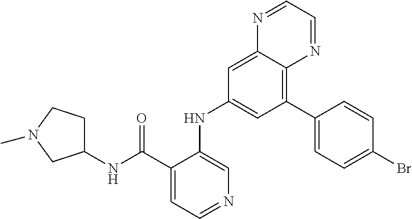 [SMILES: CN1CCC(NC(=O)c2ccncc2Nc2cc(-c3ccc(Br)cc3)c3nccnc3c2)C1]

Anhydrous copper(II) bromide (72 mg; 0.32 mmol; 1.25 eq.), tert-butyl nitrite (0.05 mL; 0.45 mmol; 1.75 eq.), and anhydrous acetonitrile (3 mL) are added to a 10-mL round-bottom flask under argon and the solution is heated to 65° C. with stirring under argon. In a separate 25-mL round-bottom flask, 3-{[8-(4-Aminophenyl)quinoxalin-6-yl]amino}-N-(1-methyl-pyrrolidin-3-yl)pyridine-4-carboxamide (Intermediate 127, 113 mg; 0.26 mmol; 1 eq.) is suspended in anhydrous acetonitrile (3 mL) and heated to 65° C. for 10 min. The solution from the first flask is then added dropwise over 5 min to the second solution using syringe techniques. The reaction mixture is left with stirring at 65° C. under argon for 1 h. After coming back to room temperature, the reaction is quenched with water and extracted with EtOAc. The organic phase is dried over sodium sulfate, filtered and concentrated to dryness. The residue is dissolved in DCM and the solution is filtered over a short pad of neutral allumina, eluting with EtOAc. The filtrate is evaporated to dryness and the resulting residue is purified by reversed-phase preparative HPLC (column: Gemini NX C18 5u 110A (100×30 mm), ACN gradient in water, 0.1% TFA). The pure fractions are pooled, MeCN is evaporated and the resulting solution is basified with NaHCO₃. It is then extracted with DCM (twice) and the combined organic layers are dried (sodium sulfate) and evaporated to dryness to yield give 3-{[8-(4-bromophenyl)quinoxalin-6-yl]amino}-N-(1-methylpyrrolidin-3-yl)pyridine-4-carboxamide (9 mg; 0.02 mmol; 7%; yellow powder; HPLC purity: 98.3%).

The following compounds can be synthesized by adapting synthetic procedures described hereinabove and utilizing appropriate starting material in a manner that is readily comprehensible to the skilled person:
3-{[8-(4-Fluoro-1-methyl-1H-indol-6-yl)quinoxalin-6-yl]amino}-N-(1-methyl-pyrrolidin-3-yl)pyridine-4-carboxamide
8-(2-amino-1-benzothiophen-6-yl)-N-(4-methanesulfonylpyridin-3-yl)quinoxalin-6-amine
3-{[8-(1-methyl-1H-indol-6-yl)quinoxalin-6-yl]oxy}pyridine-4-carboxamide
3-{[8-(3-methyl-1-benzothiophen-5-yl)quinoxalin-6-yl]amino}-N-(5-oxo-pyrrolidin-3-yl)pyridine-4-carboxamide
3-{[8-(3-methyl-1-benzothiophen-5-yl)quinoxalin-6-yl]amino}-N-(2-oxo-pyrrolidin-3-yl)pyridine-4-carboxamide
3-{[8-(3-methyl-1-benzothiophen-5-yl)quinoxalin-6-yl]amino}-N-(1-methyl-5-oxopyrrolidin-3-yl)pyridine-4-carboxamide
3-{[8-(3-methyl-1-benzothiophen-5-yl)quinoxalin-6-yl]amino}-N-(1-methyl-2-oxopyrrolidin-3-yl)pyridine-4-carboxamide
8-(1-methyl-1H-indol-6-yl)-N-{4-[(methylamino)methyl]pyridin-3-yl}quinoxalin-6-amine
N-methyl-3-{[8-(3-methyl-1-benzothiophen-5-yl)quinoxalin-6-yl]amino}-pyridine-4-carboxamide
3-{[8-(1-methyl-1H-indol-6-yl)quinoxalin-6-yl]amino}-N-(pyrimidin-4-0)-pyridine-4-carboxamide
8-(1-methyl-1H-indol-6-yl)-N-(4-{[(pyrimidin-5-yl)amino]methyl}pyridin-3-yl)quinoxalin-6-amine
2-amino-N-(5-{7-[(4-methanesulfonylpyridin-3-yl)amino]quinoxalin-5-yl}-1-benzothiophen-2-yl)acetamide
N-(5-fluoro-1-methylpyrrolidin-3-yl)-3-{[8-(3-methyl-1-benzothiophen-5-yl)quinoxalin-6-yl]amino}pyridine-4-carboxamide
N-(3-fluoro-1-methylpyrrolidin-3-yl)-3-{[8-(3-methyl-1-benzothiophen-5-yl)quinoxalin-6-yl]amino}pyridine-4-carboxamide
N-(5,5-difluoro-1-methylpyrrolidin-3-yl)-3-{[8-(3-methyl-1-benzothiophen-5-yl)quinoxalin-6-yl]amino}pyridine-4-carboxamide
N-(3,3-difluoro-1-methylpyrrolidin-3-yl)-3-{[8-(3-methyl-1-benzothiophen-5-yl)quinoxalin-6-yl]amino}pyridine-4-carboxamide.

TABLE 1
Analytical data of compounds according to the examples described
hereinabove.

| Cpd. | Ex. | | | | |
|---|---|---|---|---|---|
| No. | No. | MW | IUPAC name | LC-MS | ¹H-NMR |
| 1 | 1 | 434.47 | 8-(2,3-dihydro- | 98.7% | 1H NMR (400 MHz, DMSO): δ |
| | | | 1,4-benzodioxin- | [M + H]⁺ = 435.0 | 8.97 (s, 1H), 8.81 (d, J = 1.9 Hz, |
| | | | 6-yl)- | | 1H), 8.72 (d, J = 1.9 Hz, 1H), |
| | | | N-(4- | | 8.56 (d, J = 5.1 Hz, 1H), 8.42 (s, |
| | | | methanesulfonylpyridin- | | 1H), 7.84 (dd, J = 5.1, 0.5 Hz, |
| | | | 3- | | 1H), 7.76 (d, J = 2.6 Hz, 1H), |
| | | | yl)quinoxalin-6- | | 7.55 (d, J = 2.6 Hz, 1H), |
| | | | amine | | 7.20 (d, J = 2.1 Hz, 1H), 7.13 (dd, J = 8.4, |
| | | | | | 2.1 Hz, 1H), 6.97 (d, J = 8.3 Hz, |
| | | | | | 1H), 4.30 (s, 4H), 3.36 (s, |
| | | | | | 3H). |
| 2 | 2 | 377.45 | 5-(1-methyl-1H- | 99.4% | ¹H NMR (400 MHz, DMSO): δ |
| | | | indol-6-yl)-7- | [M + H]⁺ = 378.3 | 8.86 (d, J = 1.7 Hz, 1H), |
| | | | {1H,2H,3H- | | 8.74 (d, J = 1.8 Hz, 1H), 8.71 (s, 1H), |
| | | | pyrrolo[2,3- | | 8.10 (d, J = 4.6 Hz, 1H), |
| | | | c]pyridin-1- | | 7.93 (d, J = 2.7 Hz, 1H), 7.77 (s, 1H), |
| | | | yl}quinoxaline | | 7.66 (d, J = 2.3 Hz, 1H), |
| | | | | | 7.64 (d, J = 3.1 Hz, 1H), 7.41 (d, J = 3.0 Hz, |
| | | | | | 1H), 7.39 (dd, J = 8.2, |
| | | | | | 1.4 Hz, 1H), 7.34 (d, J = 4.3 Hz, |
| | | | | | 1H), 6.50 (d, J = 3.0 Hz, 1H), |
| | | | | | 4.25 (t, J = 8.6 Hz, 2H), 3.84 (s, |
| | | | | | 3H), 3.27 (t, J = 7.5 Hz, 2H). |
| 3 | 3 | 428.51 | N-(2- | 95.8%. | ¹H NMR (400 MHz, DMSO): δ |
| | | | methanesulfonylphenyl)- | [M + H]⁺ = 429.2 | 8.80 (d, J = 1.9 Hz, 1H), |
| | | | 8-(1- | | 8.71 (d, J = 1.8 Hz, 1H), 8.37 (s, 1H), |
| | | | methyl-1H-indol- | | 7.95 (dd, J = 8.0, 1.2 Hz, 1H), |
| | | | 6-yl)quinoxalin-6- | | 7.81 (d, J = 2.6 Hz, 1H), |
| | | | amine | | 7.73 (dd, J = 9.9, 1.6 Hz, 3H), |
| | | | | | 7.63 (dd, J = 8.2, 0.5 Hz, 1H), |
| | | | | | 7.54 (d, J = 2.6 Hz, 1H), 7.41 (d, J = 3.0 Hz, |
| | | | | | 1H), 7.34 (dt, J = 13.0, |
| | | | | | 3.7 Hz, 2H), 6.49 (dd, J = 3.1, |
| | | | | | 0.8 Hz, 1H), 3.83 (s, 3H), |
| | | | | | 3.28 (s, 3H). |
| 4 | 4 | 433.50 | 8-(1,3- | 97.6% | ¹H NMR (400 MHz, DMSO): δ |
| | | | benzothiazol-6-yl)- | [M + H]⁺ = 434.1 | 9.46 (s, 1H), 9.01 (s, 1H), |
| | | | N-(4- | | 8.85 (d, J = 1.8 Hz, 1H), 8.75 (d, J = 1.8 Hz, |
| | | | methanesulfonylpyridin- | | 1H), 8.58 (d, J = 5.1 Hz, |
| | | | 3- | | 1H), 8.49 (s, 1H), 8.46 (d, J = 1.5 Hz, |
| | | | yl)quinoxalin-6- | | 1H), 8.20 (d, J = 8.4 Hz, |
| | | | amine | | 1H), 7.91 (d, J = 2.6 Hz, 1H), |
| | | | | | 7.85 (d, J = 4.8 Hz, 1H), |
| | | | | | 7.84 (dd, J = 8.3, 1.8 Hz, 1H), |
| | | | | | 7.63 (d, J = 2.6 Hz, 1H), 3.38 (s, 3H). |
| 5 | 5 | 440.90 | 8-(2-chloro-5- | 98.1% | ¹H NMR (400 MHz, DMSO): δ |
| | | | methoxyphenyl)- | [M + H]⁺ = 440.95 | 8.95 (s, 1H), 8.82 (d, J = 1.9 Hz, |
| | | | N-(4- | | 1H), 8.68 (d, J = 1.9 Hz, 1H), |
| | | | methanesulfonylpyridin- | | 8.56 (d, J = 5.1 Hz, 1H), 8.50 (s, |
| | | | 3- | | 1H), 7.84 (d, J = 5.1 Hz, 1H), |
| | | | yl)quinoxalin-6- | | 7.70 (d, J = 2.6 Hz, 1H), |
| | | | amine | | 7.65 (d, J = 2.5 Hz, 1H), 7.48 (dd, J = 9.7, |
| | | | | | 0.5 Hz, 1H), |
| | | | | | 7.07-7.04 (m, 2H), 3.78 (s, 3H), 3.36 (s, |
| | | | | | 3H). |
| 6 | 6 | 429.49 | N-(2- | 97.6%. | ¹H NMR (400 MHz, DMSO): δ |
| | | | methanesulfonylpyridin- | [M + H]⁺ = 430.4 | 8.83 (d, J = 1.8 Hz, 1H), |
| | | | 3-yl)-8-(1- | | 8.75 (d, J = 1.8 Hz, 1H), 8.66 (s, 1H), |
| | | | methyl-1H- | | 8.41 8.30 (m, 1H), 8.22 (d, J = 9.4 Hz, |
| | | | indol-6- | | 1H), 7.81 (d, J = 2.5 Hz, |
| | | | yl)quinoxalin-6- | | 1H), 7.73 (s, 1H), |
| | | | amine | | 7.73 7.63 (m, 1H), 7.63 (dd, J = 5.3, 2.8 Hz, |
| | | | | | 2H), 7.40 (d, J = 3.0 Hz, |
| | | | | | 1H), 7.35 (d, J = 8.2 Hz, 1H), |
| | | | | | 6.48 (d, J = 3.0 Hz, 1H), 3.83 (s, |
| | | | | | 3H), 3.42 (s, 3H). |
| 7 | 7 | 499.58 | 8-(1-methyl-1H- | 98.0%. | ¹H NMR (400 MHz, DMSO): δ |
| | | | indol-6-yl)-N-[2- | [M + H]⁺ = 500.3 | 8.81 (d, J = 1.8 Hz, 1H), |
| | | | (morpholine-4- | | 8.71 (d, J = 1.8 Hz, 1H), 8.34 (s, 1H), |
| | | | sulfonyl)phenyl]quinoxalin- | | 7.85-7.82 (m, 1H), |
| | | | 6-amine | | 7.82-7.81 (m, 1H), 7.79 (d, J = 7.5 Hz, |
| | | | | | 1H), 7.74-7.67 (m, 2H), |
| | | | | | 7.63 (d, J = 8.2 Hz, 1H), |
| | | | | | 7.61 (d, J = 2.6 Hz, 1H), 7.41 (d, J = 3.1 Hz, |
| | | | | | 1H), 7.35 (dd, J = 8.2, |
| | | | | | 1.5 Hz, 1H), 7.29-7.23 (m, |
| | | | | | 1H), 6.49 (dd, J = 3.0, 0.8 Hz, |
| | | | | | 1H), 3.84 (s, 3H), |
| | | | | | 3.56-3.47 (m, 4H), 3.07-2.98 (m, 4H). |
| 8 | 8 | 429.49 | 2-{[8-(1-methyl- | 93.8%. | ¹H NMR (400 MHz, DMSO): δ |
| | | | 1H-indol-6- | [M + H] = 430.3 | 8.80 (d, J = 1.8 Hz, 1H), |
| | | | yl)quinoxalin-6- | | 8.70 (d, J = 1.9 Hz, 1H), 8.15 (s, 1H), |
| | | | yl]amino}benzene- | | 7.91 (dd, J = 8.0, 1.5 Hz, 1H), |
| | | | 1-sulfonamide | | 7.77-7.74 (m, 1H), 7.73 (d, J = 2.7 Hz, |
| | | | | | 1H), 7.72 (d, J = 0.7 Hz, |
| | | | | | 1H), 7.64 (dd, J = 8.3, 0.4 Hz, |
| | | | | | 1H), 7.62 (s, 1H), 7.60 (s, |
| | | | | | 2H), 7.56 (d, J = 2.6 Hz, 1H), |
| | | | | | 7.41 (d, J = 3.1 Hz, 1H), |
| | | | | | 7.35 (dd, J = 8.2, 1.5 Hz, 1H), |
| | | | | | 7.24-7.16 (m, 1H), 6.49 (dd, J = 3.1, |
| | | | | | 0.8 Hz, 1H), 3.84 (s, 3H). |
| 9 | 9 | 547.52 | 8-(1,3- | 99.4% | ¹H NMR (400 MHz, DMSO): δ |
| | | | benzothiazol-5-yl)- | [M + H]⁺ = 434.2 | 9.46 (s, 1H), 9.02 (s, 1H), |
| | | | N-(4- | | 8.85 (d, J = 1.7 Hz, 1H), 8.74 (d, J = 1.8 Hz, |
| | | | methanesulfonylpyridin- | | 1H), 8.57 (d, J = 5.1 Hz, |
| | | | 3- | | 1H), 8.49 (s, 1H), 8.37 (d, J = 1.2 Hz, |
| | | | yl)quinoxalin-6- | | 1H), 8.28 (d, J = 8.4 Hz, |
| | | | amine | | 1H), 7.94 (d, J = 2.5 Hz, 1H), |
| | | | trifluoroacetate | | 7.85 (d, J = 5.1 Hz, 1H), |
| | | | | | 7.80 (dd, J = 8.3, 1.5 Hz, 1H), |
| | | | | | 7.63 (d, J = 2.5 Hz, 1H), 3.38 (s, 3H). |
| 10 | 10 | 507.40 | N-(5-bromo-2- | 99.4%. | ¹H NMR (400 MHz, DMSO): δ |
| | | | methanesulfonylphenyl)- | [M + H]⁺ = 507.4 | 8.85 (d, J = 1.8 Hz, 1H), |
| | | | 8-(1- | | 8.77 (d, J = 1.8 Hz, 1H), 8.45 (s, 1H), |
| | | | methyl-1H- | | 7.86 (d, J = 2.6 Hz, 1H), 7.83 (t, |
| | | | indol-6- | | J = 5.2 Hz, 2H), 7.71 (s, 1H), |
| | | | yl)quinoxalin-6- | | 7.68 (d, J = 2.5 Hz, 1H), |
| | | | amine | | 7.64 (d, J = 8.2 Hz, 1H), 7.47 (dd, J = 8.5, |
| | | | | | 1.9 Hz, 1H), 7.41 (d, J = 3.1 Hz, |
| | | | | | 1H), 7.36 (dd, J = 8.2, 1.4 Hz, |
| | | | | | 1H), 6.49 (dd, J = 3.0, 0.6 Hz, |
| | | | | | 1H), 3.84 (s, 3H), 3.30 (s, 3H). |
| 11 | 11 | 429.49 | N-(4- | >99.0%. | ¹H NMR (400 MHz, DMSO): δ |
| | | | methanesulfonylpyridin- | [M + H]⁺ = 430.2 | 9.01 (s, 1H), 8.82 (d, J = 1.8 Hz, |
| | | | 3-yl)-8-(1- | | 1H), 8.73 (d, J = 1.8 Hz, 1H), |
| | | | methyl-1H- | | 8.57 (d, J = 5.1 Hz, 1H), 8.46 (s, |
| | | | indol-6- | | 1H), 7.87-7.83 (m, 2H), |
| | | | yl)quinoxalin-6- | | 7.72 (s, 1H), 7.63 (d, J = 8.2 Hz, 1H), |
| | | | amine | | 7.55 (d, J = 2.6 Hz, 1H), |
| | | | | | 7.41 (d, J = 3.0 Hz, 1H), 7.34 (dd, J = 8.2, |
| | | | | | 1.4 Hz, 1H), 6.49 (dd, J = 3.0, |
| | | | | | 0.6 Hz, 1H), 3.83 (s, 3H), |
| | | | | | 3.39 (s, 3H). |
| 13 | 13 | 381.43 | N-(2- | 99.7%. | ¹H NMR (400 MHz, DMSO): δ |
| | | | methoxypyridin-3- | [M + H]⁺ = 382.1 | 8.73 (d, J = 1.8 Hz, 1H), |
| | | | yl)-8-(1-methyl- | | 8.62 (d, J = 1.8 Hz, 1H), 8.46 (s, 1H), |
| | | | 1H-indol-6- | | 7.89 (dd, J = 4.9, 1.6 Hz, 1H), |
| | | | yl)quinoxalin-6- | | 7.85 (dd, J = 7.6, 1.6 Hz, 1H), |
| | | | amine | | 7.75 (d, J = 2.6 Hz, 1H), 7.68 (s, |
| | | | | | 1H), 7.63 (d, J = 8.2 Hz, 1H), |
| | | | | | 7.40 (d, J = 3.1 Hz, 1H), |
| | | | | | 7.34 (d, J = 2.7 Hz, 1H), |
| | | | | | 7.33-7.30 (m, 1H), 7.04 (dd, J = 7.6, 4.9 Hz, |
| | | | | | 1H), 6.49 (d, J = 3.0 Hz, |
| | | | | | 1H), 3.97 (s, 3H), 3.83 (s, 3H). |
| 14 | 14 | 367.40 | 3-{[8-(1-methyl- | 96.5%. | ¹H NMR (400 MHz, DMSO): δ |
| | | | 1H-indol-6- | [M + H]⁺ = 368.0 | 11.88 (s, 1H), 8.77 (d, J = 1.8 Hz, |
| | | | yl)quinoxalin-6- | | 1H), 8.65 (d, J = 1.8 Hz, |
| | | | yl]amino}pyridin- | | 1H), 8.42 (s, 1H), 7.92 (d, J = 2.6 Hz, |
| | | | 2-ol | | 1H), 7.71 (s, 1H), |
| | | | | | 7.64 (s, 1H), 7.62 (d, J = 7.2 Hz, 1H), |
| | | | | | 7.55 (dd, J = 7.2, 1.7 Hz, 1H), |
| | | | | | 7.40 (d, J = 3.1 Hz, 1H), |
| | | | | | 7.36 (dd, J = 8.2, 1.4 Hz, 1H), |
| | | | | | 7.03 (s, 1H), 6.49 (dd, J = 3.1, 0.7 Hz, |
| | | | | | 1H), 6.28 (t, J = 6.9 Hz, 1H), |
| | | | | | 3.84 (s, 3H). |
| 15 | 15 | 612.62 | 8-(1-methyl-1H- | 89.4%. | |
| | | | indol-6-yl)-N-[2- | [M + H]⁺ = 499.3 | |
| | | | (piperazine-1- | | |
| | | | sulfonyl)phenyl]quinoxalin- | | |
| | | | 6-amine | | |
| | | | trifluotroacetate | | |
| 16 | 16 | 443.52 | N-methyl-2-{[8-(1- | 90.0%. | ¹H NMR (400 MHz, DMSO): δ |
| | | | methyl-1H-indol- | [M + H]⁺ = 444.3 | 8.80 (d, J = 1.8 Hz, 1H), |
| | | | 6-yl)quinoxalin-6- | | 8.71 (d, J = 1.9 Hz, 1H), 8.15 (s, 1H), |
| | | | yl]amino}benzene- | | 7.86 (dd, J = 8.0, 1.6 Hz, 1H), |
| | | | 1-sulfonamide | | 7.77 (dd, J = 8.3, 0.9 Hz, 1H), |
| | | | | | 7.75 (d, J = 2.6 Hz, 1H), |
| | | | | | 7.73-7.72 (m, 1H), 7.69 (s, 1H), |
| | | | | | 7.67-7.62 (m, 2H), 7.57 (d, J = 2.6 Hz, |
| | | | | | 1H), 7.41 (d, J = 3.1 Hz, |
| | | | | | 1H), 7.35 (dd, J = 8.2, 1.5 Hz, |
| | | | | | 1H), 7.25-7.19 (m, 1H), |
| | | | | | 6.49 (dd, J = 3.1, 0.8 Hz, 1H), |
| | | | | | 3.84 (s, 3H), 2.47 (s, 3H). |
| 17 | 17 | 366.42 | 3-N-[-(1-methyl- | 97.9%. | ¹H NMR (400 MHz, DMSO): δ |
| | | | 1H-indol-6- | [M + H]⁺ = 367.3 | 8.68 (d, J = 1.9 Hz, 1H), |
| | | | yl)quinoxalin-6- | | 8.55 (d, J = 1.9 Hz, 1H), 8.10 (s, 1H), |
| | | | yl]pyridine-2,3- | | 7.86 (dd, J = 4.9, 1.5 Hz, 1H), |
| | | | diamine | | 7.66 (s, 1H), 7.62 (d, J = 8.2 Hz, |
| | | | | | 1H), 7.56-7.49 (m, 2H), |
| | | | | | 7.40 (d, J = 3.1 Hz, 1H), 7.30 (dd, J = 8.2, |
| | | | | | 1.4 Hz, 1H), 6.89 (d, J = 2.7 Hz, |
| | | | | | 1H), 6.65 (dd, J = 7.5, 4.9 Hz, |
| | | | | | 1H), 6.49 (d, J = 3.1 Hz, |
| | | | | | 1H), 5.82 (s, 2H), 3.83 (s, 3H). |
| 18 | 18 | 376.41 | 3-{[8-(1-methyl- | 97.3%. | ¹H NMR (400 MHz, DMSO): δ |
| | | | 1H-indol-6- | [M + H]⁺ = 377.3 | 9.47 (s, 1H), 8.93 (s, 1H), |
| | | | yl)quinoxalin-6- | | 8.82 (d, J = 1.8 Hz, 1H), 8.73 (d, J = 1.8 Hz, |
| | | | yl]amino}pyridine- | | 1H), 8.42 (d, J = 5.0 Hz, |
| | | | 4-carbonitrile | | 1H), 7.84 (dd, J = 5.0, 0.6 Hz, |
| | | | | | 1H), 7.78-7.69 (m, 2H), |
| | | | | | 7.64 (d, J = 8.2 Hz, 1H), 7.50 (d, J = 2.6 Hz, |
| | | | | | 1H), 7.41 (d, J = 3.1 Hz, |
| | | | | | 1H), 7.34 (dd, J = 8.2, 1.5 Hz, |
| | | | | | 1H), 6.49 (dd, J = 3.0, 0.8 Hz, |
| | | | | | 1H), 3.83 (s, 3H). |
| 19 | 19 | 394.43 | 3-{[8-(1-methyl- | >92.0%. | ¹H NMR (400 MHz, DMSO) δ |
| | | | 1H-indol-6- | [M + H]⁺ = 395.3 | 9.63 (s, 1H), 8.93 (s, 1H), |
| | | | yl)quinoxalin-6- | | 8.79 (d, J = 1.6 Hz, 1H), 8.69 (d, J = 1.7 Hz, |
| | | | yl]amino}pyridine- | | 1H), 8.30 (d + s, J = 5.1 Hz, |
| | | | 4-carboxamide | | 2H), 7.86 (s, 1H), 7.72 (s, |
| | | | | | 1H), 7.71 (d, J = 2.5 Hz, 1H), |
| | | | | | 7.63 (d + d, J = 7.7, 4.0 Hz, 2H), |
| | | | | | 7.55 (d, J = 2.5 Hz, 1H), |
| | | | | | 7.40 (d, J = 3.0 Hz, 1H), 7.34 (dd, J = 8.2, |
| | | | | | 1.1 Hz, 1H), 6.48 (d, J = 2.9 Hz, |
| | | | | | 1H), 3.83 (s, 3H). |
| 20 | 20 | 457.55 | N,N-dimethyl-2- | >99.5%. | ¹H NMR (400 MHz, DMSO): δ |
| | | | {[8-(1-methyl-1H- | [M + H]⁺ = 458.1 | 8.79 (d, J = 1.8 Hz, 1H), |
| | | | indol-6- | | 8.70 (d, J = 1.8 Hz, 1H), 8.40 (s, 1H), |
| | | | yl)quinoxalin-6- | | 7.84-7.82 (m, 1H), 7.81 (d, J = 1.2 Hz, |
| | | | yl]amino}benzene- | | 1H), 7.79 (s, 1H), |
| | | | 1-sulfonamide | | 7.72 (s, 1H), 7.72-7.66 (m, 1H), |
| | | | | | 7.63 (d, J = 7.8 Hz, 1H), |
| | | | | | 7.58 (d, J = 2.6 Hz, 1H), 7.41 (d, J = 3.1 Hz, |
| | | | | | 1H), 7.34 (dd, J = 8.2, |
| | | | | | 1.5 Hz, 1H), 7.30-7.24 (m, |
| | | | | | 1H), 6.49 (dd, J = 3.1, 0.8 Hz, |
| | | | | | 1H), 3.84 (s, 3H), 2.67 (s, 6H). |
| 21 | 21 | 429.49 | N-(2- | >99.5%. | |
| | | | methanesulfonylphenyl)- | [M + H]⁺ = 430.3 | |
| | | | 8-{1- | | |
| | | | methyl-1H- | | |
| | | | pyrrolo[2,3- | | |
| | | | b]pyridin-6- | | |
| | | | yl}quinoxalin-6- | | |
| | | | amine | | |
| | | | trifluoroacetate | | |
| 22 | 22 | 430.48 | N-(4- | 93.5%. | ¹H NMR (400 MHz, DMSO): δ |
| | | | methanesulfonylpyridin- | [M + H]⁺ = 431.1 | 9.00 (s, 1H), 8.82 (d, J = 1.9 Hz, |
| | | | 3-yl)-8-(3- | | 1H), 8.73 (d, J = 1.9 Hz, 1H), |
| | | | methyl-1- | | 8.57 (d, J = 5.1 Hz, 1H), 8.46 (s, |
| | | | benzofuran-5- | | 1H), 7.86 (d, J = 2.6 Hz, 1H), |
| | | | yl)quinoxalin-6- | | 7.85-7.82 (m, 3H), 7.64 (dd, J = 8.5, |
| | | | amine | | 0.6 Hz, 1H), |
| | | | | | 7.61-7.57 (m, J = 8.6, 2.6, 1.7 Hz, 2H), |
| | | | | | 3.38 (s, 3H), 2.25 (d, J = 1.3 Hz, |
| | | | | | 3H). |
| 23 | 23 | 381.43 | N-(4- | 99.3%. | ¹H NMR (400 MHz, DMSO): δ |
| | | | methoxypyridin-3- | [M + H]⁺ = 382.3 | 8.70 (d, J = 1.8 Hz, 1H), |
| | | | yl)-8-(1-methyl- | | 8.57 (d, J = 1.9 Hz, 1H), 8.54 (s, 1H), |
| | | | 1H-indol-6- | | 8.46 (s, 1H), 8.31 (d, J = 5.6 Hz, |
| | | | yl)quinoxalin-6- | | 1H), 7.66 (s, 1H), |
| | | | amine | | 7.64-7.59 (m, 2H), 7.40 (d, J = 3.0 Hz, |
| | | | | | 1H), 7.29 (dd, J = 8.2, 1.5 Hz, |
| | | | | | 1H), 7.21 (d, J = 5.6 Hz, 1H), |
| | | | | | 7.05 (d, J = 2.6 Hz, 1H), |
| | | | | | 6.48 (dd, J = 3.1, 0.8 Hz, 1H), |
| | | | | | 3.92 (s, 3H), 3.82 (s, 3H). |
| 24 | 24 | 377.41 | 3-{[8-(3-methyl-1- | >99.5% | ¹H NMR (400 MHz, DMSO): δ |
| | | | benzofuran-5-yl)quinoxalin- | [M + H]⁺ = 378.2 | 9.47 (s, 1H), 8.93 (s, 1H), |
| | | | 6-yl]amino}pyridine- | | 8.83 (d, J = 1.8 Hz, 1H), 8.74 (d, J = 1.8 Hz, |
| | | | 4-carbonitrile | | 1H), 8.42 (d, J = 5.0 Hz, |
| | | | | | 1H), 7.84 (dd, J = 3.4, 1.6 Hz, |
| | | | | | 3H), 7.76 (d, J = 2.6 Hz, 1H), |
| | | | | | 7.65 (dd, J = 8.5, 0.5 Hz, 1H), |
| | | | | | 7.59 (dd, J = 8.5, 1.7 Hz, 1H), |
| | | | | | 7.53 (d, J = 2.6 Hz, 1H), |
| | | | | | 2.25 (d, J = 1.3 Hz, 3H). |
| 25 | 25 | 453.52 | 4- | >99.5% | ¹H NMR (400 MHz, DMSO): δ |
| | | | methanesulfonyl- | [M + H]⁺ = 454.2 | 8.86 (d, J = 1.8 Hz, 1H), |
| | | | 3-{[8-(1-methyl- | | 8.78 (d, J = 1.8 Hz, 1H), 8.52 (s, 1H), |
| | | | 1H-indol-6- | | 8.16 (d, J = 1.4 Hz, 1H), |
| | | | yl)quinoxalin-6- | | 8.05 (d, J = 8.2 Hz, 1H), 7.88 (d, J = 2.5 Hz, |
| | | | yl]amino}benzonitrile | | 1H), 7.73 (d, J = 2.6 Hz, |
| | | | | | 1H), 7.72 (s, 1H), 7.67 (dd, J = 8.2, |
| | | | | | 1.5 Hz, 1H), 7.64 (d, J = 8.2 Hz, |
| | | | | | 1H), 7.41 (d, J = 3.1 Hz, |
| | | | | | 1H), 7.37 (dd, J = 8.2, 1.4 Hz, |
| | | | | | 1H), 6.49 (dd, J = 3.1, 0.7 Hz, |
| | | | | | 1H), 3.84 (s, 3H), 3.37 (s, 3H). |
| 26 | 26 | 395.42 | 3-{[8-(3-methyl-1- | 98.9% | |
| | | | benzofuran-5- | [M + H]⁺ = 396.2 | |
| | | | yl)quinoxalin-6- | | |
| | | | yl]amino}pyridine- | | |
| | | | 4-carboxamide | | |
| 27 | 27 | 430.49 | N-(5-methanesulfonylpyrimidin- | >99.5% | ¹H NMR (400 MHz, DMSO): δ |
| | | | 4-yl)- | [M + H]⁺ = 431.2 | 9.57 (s, 1H), 8.91 (d, J = 1.8 Hz, |
| | | | 8-(1-methyl-1H-indol- | | 1H), 8.82 (d, J = 1.8 Hz, 1H), |
| | | | 6-yl)quinoxalin- | | 8.69 (d, J = 2.4 Hz, 1H), |
| | | | 6-amine | | 8.62 (d, J = 2.5 Hz, 1H), 8.33 (d, J = 2.4 Hz, |
| | | | | | 1H), 7.94 (d, J = 2.5 Hz, |
| | | | | | 1H), 7.75 (s, 1H), 7.64 (d, J = 8.2 Hz, |
| | | | | | 1H), 7.41 (d, J = 3.1 Hz, |
| | | | | | 1H), 7.37 (dd, J = 8.2, 1.5 Hz, |
| | | | | | 1H), 6.50 (dd, J = 3.0, 0.8 Hz, |
| | | | | | 1H), 3.85 (s, 3H), 3.50 (s, 3H). |
| 28 | 28 | 376.42 | 3-{[8-(1-methyl- | 95.4% | ¹H NMR (400 MHz, DMSO): δ |
| | | | 1H-indol-5- | [M + H]⁺ = 337.2 | 9.44 (s, 1H), 8.93 (s, 1H), |
| | | | yl)quinoxalin-6- | | 8.81 (d, J = 1.8 Hz, 1H), 8.73 (d, J = 1.8 Hz, |
| | | | yl]amino}pyridine- | | 1H), 8.41 (d, J = 5.0 Hz, |
| | | | 4-carbonitrile | | 1H), 7.85 (dd, J = 1.6, 0.6 Hz, |
| | | | | | 1H), 7.84 (dd, J = 5.1, 0.7 Hz, |
| | | | | | 1H), 7.72 (d, J = 2.6 Hz, 1H), |
| | | | | | 7.54 (d, J = 8.5 Hz, 1H), |
| | | | | | 7.49 (d, J = 2.6 Hz, 1H), 7.47 (dd, J = 8.5, |
| | | | | | 1.6 Hz, 1H), 7.39 (d, J = 3.1 Hz, |
| | | | | | 1H), 6.51 (dd, J = 3.1, 0.8 Hz, |
| | | | | | 1H), 3.85 (s, 3H). |
| 29 | 29 | 394.44 | 3-{[8-(1-methyl- | 97.7% | ¹H NMR (400 MHz, DMSO) δ |
| | | | 1H-indol-5- | [M + H]⁺ = 395.2 | 9.61 (s, 1H), 8.93 (s, 1H), |
| | | | yl)quinoxalin-6- | | 8.78 (d, J = 1.9 Hz, 1H), 8.68 (d, J = 1.9 Hz, |
| | | | yl]amino}pyridine- | | 1H), 8.30 (d + s, J = 5.0 Hz, |
| | | | 4-carboxamide | | 2H), 7.87-7.83 (m, 2H), |
| | | | | | 7.67 (d, J = 2.6 Hz, 1H), |
| | | | | | 7.63 (d, J = 5.0 Hz, 1H), 7.53 (d + dt, J = 8.5, |
| | | | | | 2.6 Hz, 2H), 7.47 (dd, J = 8.5, |
| | | | | | 1.6 Hz, 1H), 7.38 (d, J = 3.1 Hz, |
| | | | | | 1H), 6.50 (dd, J = 3.1, 0.7 Hz, |
| | | | | | 1H), 3.85 (s, 3H). |
| 30 | 30 | 385.85 | N-(4- | 97.3% | ¹H NMR (400 MHz, DMSO): δ |
| | | | chloropyridin-3- | [M + H]⁺ = 386.4 | 8.80 (s, 1H), 8.79 (s, 1H), |
| | | | yl)-8-(1-methyl- | | 8.75 (d, J = 1.9 Hz, 1H), 8.65 (d, J = 1.8 Hz, |
| | | | 1H-indol-5- | | 1H), 8.33 (d, J = 5.2 Hz, |
| | | | yl)quinoxalin-6- | | 1H), 7.84-7.81 (m, 1H), |
| | | | amine | | 7.68 (d, J = 5.3 Hz, 1H), 7.64 (d, J = 2.6 Hz, |
| | | | | | 1H), 7.54 (d, J = 8.5 Hz, |
| | | | | | 1H), 7.44 (dd, J = 8.5, 1.6 Hz, |
| | | | | | 1H), 7.39 (d, J = 3.1 Hz, 1H), |
| | | | | | 7.16 (d, J = 2.6 Hz, 1H), |
| | | | | | 6.51 (dd, J = 3.1, 0.8 Hz, 1H), |
| | | | | | 3.85 (s, 3H). |
| 31 | 31 | 431.5 | 8-(1-methyl-1H- | 90.1% | ¹H NMR (400 MHz, DMSO): δ |
| | | | indol-5-yl)-N-[4- | [M + H]⁺ = 432.2 | 8.65 (d, J = 1.9 Hz, 1H), |
| | | | (1-methyl-1H- | | 8.60 (d, J = 4.9 Hz, 2H), 8.55 (d, J = 1.9 Hz, |
| | | | pyrazol-4- | | 1H), 8.43 (d, J = 5.2 Hz, |
| | | | yl)pyridin-3- | | 1H), 8.27 (s, 1H), 7.98 (d, J = 0.7 Hz, |
| | | | yl]quinoxalin-6- | | 1H), 7.79 (d, J = 1.0 Hz, |
| | | | amine | | 1H), 7.71 (d, J = 5.2 Hz, 1H), |
| | | | | | 7.52 (t, J = 5.5 Hz, 2H), |
| | | | | | 7.41 (dd, J = 8.5, 1.6 Hz, 1H), |
| | | | | | 7.39 (d, J = 3.1 Hz, 1H), 6.76 (d, J = 2.6 Hz, |
| | | | | | 1H), 6.51 (dd, J = 3.0, |
| | | | | | 0.7 Hz, 1H), 3.85 (s, 3H), |
| | | | | | 3.82 (s, 3H). |
| 32 | 32 | 449.56 | 8-(1-methyl-1H- | 97.5% | ¹H NMR (400 MHz, DMSO): δ |
| | | | indol-5-yl)-N-[4- | [M + H]⁺ = 450.1 | 8.70 (d, J = 1.9 Hz, 1H), |
| | | | (4- | | 8.57 (d, J = 1.8 Hz, 1H), 8.50 (s, 1H), |
| | | | methylpiperazin- | | 8.36 (s, 1H), 8.21 (d, J = 5.5 Hz, |
| | | | 1-yl)pyridin-3- | | 1H), 7.79 (d, J = 1.1 Hz, 1H), |
| | | | yl]quinoxalin-6- | | 7.52 (d, J = 8.5 Hz, 1H), |
| | | | amine | | 7.47 (d, J = 2.6 Hz, 1H), 7.42 (dd, J = 8.5, |
| | | | | | 1.6 Hz, 1H), 7.38 (d, J = 3.1 Hz, |
| | | | | | 1H), 6.99 (d, J = 5.6 Hz, |
| | | | | | 1H), 6.94 (d, J = 2.6 Hz, 1H), |
| | | | | | 6.50 (dd, J = 3.1, 0.8 Hz, 1H), |
| | | | | | 3.85 (s, 3H), 3.20-3.08 (m, |
| | | | | | 4H), 2.18 (s, 4H), 2.01 (s, 3H). |
| 33 | 33 | 429.49 | 8-(1-methyl-1H- | 87.8% | ¹H NMR (400 MHz, DMSO): δ |
| | | | indol-5-yl)-N-[4- | [M + H]⁺ = 430.2 | 9.15 (s, 1H), 9.02 (s, 2H), |
| | | | (pyrimidin-5- | | 8.83 (s, 1H), 8.71 (s, 1H), 8.70 (d, J = 1.9 Hz, |
| | | | yl)pyridin-3- | | 1H), 8.59 (d, J = 1.9 Hz, |
| | | | yl]quinoxalin-6- | | 1H), 8.55 (d, J = 5.0 Hz, 1H), |
| | | | amine | | 7.76 (d, J = 1.1 Hz, 1H), |
| | | | | | 7.64 (d, J = 5.0 Hz, 1H), 7.51 (d, J = 8.6 Hz, |
| | | | | | 1H), 7.46 (d, J = 2.6 fHz, |
| | | | | | 1H), 7.39 (d, J = 2.7 Hz, |
| | | | | | 1H), 7.38-7.35 (m, 1H), |
| | | | | | 7.00 (d, J = 2.6 Hz, 1H), 6.49 (dd, J = 3.1, |
| | | | | | 0.7 Hz, 1H), 3.84 (s, 3H). |
| 34 | 34 | 377.45 | 5-(1-methyl-1H- | 98.1% | ¹H NMR (400 MHz, DMSO): δ |
| | | | indol-5-yl)-7- | [M + H]⁺ = 378.3 | 8.85 (d, J = 1.8 Hz, 1H), |
| | | | {1H,2H,3H- | | 8.73 (d, J = 1.8 Hz, 1H), 8.71 (s, 1H), |
| | | | pyrrolo[2,3- | | 8.10 (d, J = 4.6 Hz, 1H), |
| | | | c]pyridin-1- | | 7.90 (dd, J = 4.3, 1.8 Hz, 2H), |
| | | | yl}quinoxaline | | 7.62 (d, J = 2.7 Hz, 1H), 7.56 (d, J = 8.5 Hz, |
| | | | | | 1H), 7.52 (dd, J = 8.5, |
| | | | | | 1.6 Hz, 1H), 7.40 (d, J = 3.1 Hz, |
| | | | | | 1H), 7.34 (d, J = 3.9 Hz, 1H), |
| | | | | | 6.53 (dd, J = 3.1, 0.6 Hz, 1H), |
| | | | | | 4.24 (t, J = 8.6 Hz, 2H), 3.86 (s, |
| | | | | | 3H), 3.26 (t, J = 8.3 Hz, 2H). |
| 35 | 35 | 473.51 | N-(2- | 94.7% | ¹H NMR (400 MHz, DMSO): δ |
| | | | methanesulfonyl- | [M + H]⁺ = 444.1 | 8.88 (d, J = 1.8 Hz, 1H), |
| | | | 5-nitrophenyl)-8- | | 8.81 (d, J = 1.8 Hz, 1H), 8.66 (s, 1H), |
| | | | (1-methylindol-6- | | 8.35 (d, J = 2.2 Hz, 1H), |
| | | | yl)quinoxalin-6- | | 8.15 (d, J = 8.7 Hz, 1H), |
| | | | amine | | 7.98-7.93 (m, 2H), 7.86 (d, J = 2.5 Hz, |
| | | | | | 1H), 7.73 (s, 1H), 7.64 (dd, J = 8.2, |
| | | | | | 0.4 Hz, 1H), 7.41 (d, J = 3.1 Hz, |
| | | | | | 1H), 7.38 (dd, J = 8.2, 1.5 Hz, |
| | | | | | 1H), 6.49 (dd, J = 3.1, 0.8 Hz, |
| | | | | | 1H), 3.83 (s, 3H), 3.41 (s, |
| | | | | | 3H). |
| 36 | 36 | 443.53 | 6- | 99.0% | ¹H NMR (400 MHz, DMSO): δ |
| | | | methanesulfonyl- | [M + H]⁺ = 444.1 | 8.80 (d, J = 1.8 Hz, 1H), |
| | | | N-1-[8-(1-methyl- | | 8.70 (d, J = 1.8 Hz, 1H), 8.18 (s, 1H), |
| | | | 1H-indol-6- | | 7.76 (d, J = 2.6 Hz, 1H), |
| | | | yl)quinoxalin-6- | | 7.75-7.73 (m, 1H), 7.63 (dd, J = 8.2, |
| | | | yl]benzene-1,3- | | 0.5 Hz, 1H), 7.59 (d, J = 2.6 Hz, |
| | | | diamine | | 1H), 7.53 (d, J = 8.7 Hz, 1H), |
| | | | | | 7.40 (d, J = 3.1 Hz, 1H), |
| | | | | | 7.36 (dd, J = 8.2, 1.5 Hz, 1H), |
| | | | | | 6.84 (d, J = 2.1 Hz, 1H), 6.49 (dd, J = 3.1, |
| | | | | | 0.8 Hz, 1H), 6.38 (dd, J = 8.7, |
| | | | | | 2.1 Hz, 1H), 6.19 (s, 2H), |
| | | | | | 3.84 (s, 3H), 3.10 (s, 3H). |
| 37 | 37 | 418.47 | 8-(2,3-dihydro-1- | 98.5% | ¹H NMR (400 MHz, DMSO): δ |
| | | | benzofuran-5-yl)- | [M + H]⁺ = 419.2 | 8.97 (s, 1H), 8.81 (d, J = 1.8 Hz, |
| | | | N-(4-methane | | 1H), 8.72 (d, J = 1.8 Hz, 1H), |
| | | | sulfonylpyridin-3- | | 8.55 (d, J = 5.1 Hz, 1H), 8.41 (s, |
| | | | yl)quinoxalin-6- | | 1H), 7.83 (d, J = 5.1 Hz, 1H), |
| | | | amine | | 7.75 (d, J = 2.6 Hz, 1H), |
| | | | | | 7.54 (overlapping s and d, J = 2.4 Hz, |
| | | | | | 1H and 1H), 7.39 (dd, J = 8.2, |
| | | | | | 1.9 Hz, 1H), 6.88 (d, J = 8.2 Hz, |
| | | | | | 1H), 4.59 (t, J = 8.7 Hz, 2H), |
| | | | | | 3.37 (s, 3H), 3.25 (t, J = 8.7 Hz, |
| | | | | | 2H). |
| 38 | 38 | 457.55 | N-[5- | 87.8% | ¹H NMR (400 MHz, DMSO): δ |
| | | | (aminomethyl)-2- | [M + H]⁺ = 458.2 | 8.79 (d, J = 1.8 Hz, 1H), |
| | | | methane | | 8.70 (d, J = 1.8 Hz, 1H), 8.33 (s, 1H), |
| | | | sulfonylphenyl]-8- | | 7.88-7.84 (m, 2H), 7.77 (d, J = 2.5 Hz, |
| | | | (1-methyl-1H- | | 2H), 7.54 (d, J = 3.9 Hz, |
| | | | indol-5- | | 1H), 7.52 (d, J = 1.8 Hz, 1H), |
| | | | yl)quinoxalin-6- | | 7.49 (dd, J = 8.5, 1.5 Hz, 1H), |
| | | | amine | | 7.39 (d, J = 3.0 Hz, 1H), |
| | | | | | 7.27 (d, J = 8.7 Hz, 1H), 6.50 (d, J = 3.1 Hz, |
| | | | | | 1H), 3.85 (s, 3H), |
| | | | | | 3.80 (s, 2H), 3.23 (s, 3H). |
| 39 | 39 | 404.49 | 8-(2,5- | 98.8% | ¹H NMR (400 MHz, DMSO): δ |
| | | | dimethylphenyl)- | [M + H]⁺ = 405.2 | 8.95 (s, 1H), 8.80 (d, J = 1.7 Hz, |
| | | | N-(4-methane | | 1H), 8.65 (d, J = 1.8 Hz, 1H), |
| | | | sulfonylpyridin-3- | | 8.55 (d, J = 5.0 Hz, 1H), 8.44 (s, |
| | | | yl)quinoxalin-6- | | 1H), 7.83 (d, J = 5.1 Hz, 1H), |
| | | | amine | | 7.60 (dd, J = 9.1, 2.5 Hz, 2H), |
| | | | | | 7.17 (dd, J = 20.9, 7.9 Hz, 2H), |
| | | | | | 7.06 (s, 1H), 3.36 (s, 3H), |
| | | | | | 2.31 (s, 3H), 1.95 (s, 3H). |
| 40 | 40 | 449.56 | 8-(1-methyl-1H- | 97.5% | ¹H NMR (400 MHz, DMSO): δ |
| | | | indol-6-yl)-N-[4- | [M + H]⁺ = 450.2 | 8.70 (d, J = 1.8 Hz, 1H), |
| | | | (4- | | 8.57 (d, J = 1.8 Hz, 1H), 8.53 (s, 1H), |
| | | | methylpiperazin- | | 8.35 (s, 1H), 8.20 (d, J = 5.5 Hz, |
| | | | 1-yl)pyridin-3- | | 1H), 7.64 (s, 1H), 7.61 (d, J = 8.2 Hz, |
| | | | yl]quinoxalin-6- | | 1H), 7.48 (d, J = 2.6 Hz, |
| | | | amine | | 1H), 7.39 (d, J = 3.1 Hz, 1H), |
| | | | | | 7.28 (dd, J = 8.2, 1.4 Hz, 1H), |
| | | | | | 6.99 (d, J = 5.6 Hz, 1H), |
| | | | | | 6.95 (d, J = 2.6 Hz, 1H), 6.47 (d, J = 2.4 Hz, |
| | | | | | 1H), 3.81 (s, 3H), |
| | | | | | 3.15 (s, 4H), 2.18 (s, 4H), 2.00 (s, |
| | | | | | 3H). |
| 41 | 41 | 485.56 | N-(4- | 93.6% | ¹H NMR (400 MHz, DMSO): δ |
| | | | methanesulfonyl- | [M + H]⁺ = 486.0 | 10.38 (s, 1H), 8.83 (d, J = 1.8 Hz, |
| | | | 3-{[8-(1-methyl- | | 1H), 8.74 (d, J = 1.8 Hz, |
| | | | 1H-indol-6-yl)quinoxalin- | | 1H), 8.36 (s, 1H), 8.17 (d, J = 1.9 Hz, |
| | | | 6- | | 1H), 7.84 (s, 1H), |
| | | | yl]amino}phenyl)acetamide | | 7.82 (d, J = 5.7 Hz, 1H), 7.80 (s, 1H), |
| | | | | | 7.67 (d, J = 2.6 Hz, 1H), |
| | | | | | 7.62 (d, J = 8.2 Hz, 1H), 7.40 (d, J = 3.1 Hz, |
| | | | | | 1H), 7.37 (ddd, J = 8.8, |
| | | | | | 4.3, 1.7 Hz, 2H), 6.48 (dd, J = 3.0, |
| | | | | | 0.8 Hz, 1H), 3.84 (s, 3H), |
| | | | | | 3.22 (s, 3H), 2.08 (s, 3H). |
| 42 | 42 | 494.57 | N-[5-(1H-imidazol- | 91.5% | ¹H NMR (400 MHz, DMSO): δ |
| | | | 1-yl)-2- | [M + H]⁺ = 495.1 | 8.81 (d, J = 1.8 Hz, 1H), |
| | | | methanesulfonylphenyl]- | | 8.73 (d, J = 1.8 Hz, 1H), 8.52 (s, 1H), |
| | | | 8-(1- | | 8.44 (s, 1H), 8.05 (d, J = 2.1 Hz, |
| | | | methyl-1H-indol- | | 1H), 8.01 (d, J = 8.7 Hz, 1H), |
| | | | 6-yl)quinoxalin-6- | | 7.89 (dd, J = 3.8, 2.1 Hz, 2H), |
| | | | amine | | 7.70-7.59 (m, 4H), 7.40 (d, J = 3.0 Hz, |
| | | | | | 1H), 7.35 (dd, J = 8.2, |
| | | | | | 1.4 Hz, 1H), 7.13 (s, 1H), |
| | | | | | 6.48 (dd, J = 3.1, 0.8 Hz, 1H), |
| | | | | | 3.79 (s, 3H), 3.31 (s, 3H). |
| 43 | 43 | 496.55 | N-[2- | 90.75% | ¹H NMR (400 MHz, DMSO): |
| | | | methanesulfonyl- | [M + H]⁺ = 497.2 | 8.85 (d, J = 1.8 Hz, 1H), |
| | | | 5-(2H-1,2,3,4- | | 8.77 (d, J = 1.8 Hz, 1H), 8.53 (s, 1H), |
| | | | tetrazol-5- | | 8.39 (d, J = 1.5 Hz, 1H), |
| | | | yl)phenyl]-8-(1- | | 8.13 (d, J = 8.3 Hz, 1H), |
| | | | methyl-1H-indol- | | 7.94-7.90 (m, 2H), 7.75 (d, J = 2.6 Hz, |
| | | | 6-yl)quinoxalin-6- | | 1H), 7.72 (s, 1H), |
| | | | amine | | 7.64-7.60 (m, 1H), 7.41-7.37 (m, 2H), |
| | | | | | 6.48 (dd, J = 3.1, 0.8 Hz, 1H), |
| | | | | | 3.78 (s, 3H), 3.36 (s, 3H). |
| 44 | 44 | 445.49 | 4- | 97.20% | ¹H NMR (400 MHz, DMSO): δ |
| | | | methanesulfonyl- | [M + H]⁺ = 446.3 | 8.88 (d, J = 1.8 Hz, 1H), |
| | | | 3-{[8-(1-methyl- | | 8.80 (d, J = 1.8 Hz, 1H), 8.45 (s, 1H), |
| | | | 1H-indol-6- | | 8.37 (d, J = 1.6 Hz, 1H), |
| | | | yl)quinoxalin-6- | | 8.02 (dd, J = 6.8, 1.7 Hz, 1H), |
| | | | yl]amino}pyridin- | | 7.92 (d, J = 2.5 Hz, 1H), 7.83 (d, J = 2.5 Hz, |
| | | | 1-ium-1-olate | | 1H), 7.77 (d, J = 6.8 Hz, |
| | | | | | 1H), 7.72 (d, J = 0.5 Hz, 1H), |
| | | | | | 7.64 (d, J = 8.2 Hz, 1H), |
| | | | | | 7.41 (d, J = 3.0 Hz, 1H), 7.35 (dd, J = 8.2, |
| | | | | | 1.5 Hz, 1H), 6.49 (dd, J = 3.0, |
| | | | | | 0.7 Hz, 1H), 3.83 (s, 3H), |
| | | | | | 3.38 (s, 3H). |
| 45 | 45 | 526.66 | N-[2- | 96.7% | ¹H NMR (400 MHz, DMSO) δ |
| | | | methanesulfonyl- | [M + H]⁺ = 527.25 | 8.77 (s, 1H), 8.66 (s, 1H), |
| | | | 5-(4- | | 8.25 (s, 1H), 7.76 (s, 1H), 7.68 (d, J = 10.2 Hz, |
| | | | methylpiperazin- | | 1H), 7.67 (s, 1H), |
| | | | 1-yl)phenyl]-8-(1- | | 7.61 (d, J = 8.2 Hz, 1H), 7.51 (d, J = 1.1 Hz, |
| | | | methyl-1H-indol- | | 1H), 7.39 (d, J = 2.5 Hz, |
| | | | 6-yl)quinoxalin-6- | | 1H), 7.30 (d, J = 7.7 Hz, 1H), |
| | | | amine | | 7.09 (s, 1H), 6.84 (d, J = 8.8 Hz, |
| | | | | | 1H), 6.47 (d, J = 2.0 Hz, 1H), |
| | | | | | 3.82 (s, 3H), 3.31-3.25 (m, |
| | | | | | 4H), 3.12 (s, 3H), |
| | | | | | 2.44-2.36 (m, 4H), 2.18 (s, 3H). |
| 46 | 46 | 554.67 | 1-[4-(4- | 97.0% | ¹H NMR (400 MHz, DMSO) δ |
| | | | methanesulfonyl- | [M + H]⁺ = 555.15 | 8.78 (d, J = 1.8 Hz, 1H), |
| | | | 3-{[8-(1-methyl- | | 8.68 (d, J = 1.8 Hz, 1H), 8.28 (s, 1H), |
| | | | 1H-indol-6- | | 7.78 (d, J = 2.6 Hz, 1H), |
| | | | yl)quinoxalin-6- | | 7.71 (d, J = 9.0 Hz, 1H), 7.67 (s, 1H), |
| | | | yl]amino}phenyl)piperazin- | | 7.62 (d, J = 8.2 Hz, 1H), |
| | | | 1- | | 7.53 (d, J = 2.5 Hz, 1H), 7.40 (d, J = 3.1 Hz, |
| | | | yl]ethan-1-one | | 1H), 7.31 (dd, J = 8.2, |
| | | | | | 1.4 Hz, 1H), 7.11 (d, J = 2.3 Hz, |
| | | | | | 1H), 6.85 (dd, J = 9.1, 2.2 Hz, |
| | | | | | 1H), 6.48 (d, J = 3.0 Hz, 1H), |
| | | | | | 3.82 (s, 3H), 3.58-3.53 (m, |
| | | | | | 4H), 3.41-3.30 (m, 4H), |
| | | | | | 3.13 (s, 3H), 2.01 (s, 3H). |
| 47 | 47 | 377.40 | 3-{[8-(1-methyl- | 88.8% | ¹H NMR (400 MHz, DMSO) δ |
| | | | 1H-indol-6- | [M + H]⁺ = 378.2 | 8.97 (d, J = 1.8 Hz, 1H), |
| | | | yl)quinoxalin-6- | | 8.94 (d, J = 1.8 Hz, 1H), 8.74 (s, 1H), |
| | | | yl]oxy}pyridine-4- | | 8.68 (d, J = 4.9 Hz, 1H), |
| | | | carbonitrile | | 8.06 (dd, J = 4.9, 0.7 Hz, 1H), |
| | | | | | 7.91 (d, J = 2.8 Hz, 1H), 7.78 (s, 1H), |
| | | | | | 7.68 (d, J = 2.8 Hz, 1H), |
| | | | | | 7.64 (dd, J = 8.2, 0.5 Hz, 1H), |
| | | | | | 7.42 (d, J = 3.0 Hz, 1H), |
| | | | | | 7.40-7.37 (m, 1H), 6.49 (dd, J = 3.1, 0.8 Hz, |
| | | | | | 1H), 3.84 (s, 3H). |
| 48 | 48 | 443.49 | N-(4- | 96.8% | ¹H NMR (400 MHz, DMSO) δ |
| | | | methanesulfonylpyridin- | [M + H]⁺ = 444.1 | 9.01 (s, 1H), 8.84 (d, J = 1.8 Hz, |
| | | | 3-yl)-8-[3- | | 1H), 8.74 (d, J = 1.8 Hz, 1H), |
| | | | (1H-1,2,3-triazol- | | 8.58 (d, J = 5.1 Hz, 1H), 8.49 (s, |
| | | | 4- | | 1H), 8.43-8.27 (m, 1H), 8.14 (s, |
| | | | yl)phenyl]quinoxalin- | | 1H), 7.94 (d, J = 7.5 Hz, 1H), |
| | | | 6-amine | | 7.89 (d, J = 2.5 Hz, 1H), |
| | | | | | 7.85 (d, J = 5.1 Hz, 1H), |
| | | | | | 7.67-7.53 (m, 3H), 3.38 (s, 3H). |
| 49 | 49 | 457.55 | N-(4- | 97.7% | ¹H NMR (400 MHz, DMSO) δ |
| | | | methanesulfonylpyridin- | [M + H]⁺ = 458.4 | 9.02 (s, 1H), 8.81 (d, J = 1.8 Hz, |
| | | | 3-yl)-8-[1- | | 1H), 8.72 (d, J = 1.8 Hz, 1H), |
| | | | (propan-2-yl)-1H- | | 8.57 (d, J = 5.1 Hz, 1H), 8.46 (s, |
| | | | indol-6- | | 1H), 7.86 (d, J = 2.6 Hz, 1H), |
| | | | yl]quinoxalin-6- | | 7.85 (d, J = 5.1 Hz, 1H), 7.78 (s, |
| | | | amine | | 1H), 7.63 (d, J = 8.3 Hz, 1H), |
| | | | | | 7.57 (d, J = 3.2 Hz, 1H), |
| | | | | | 7.54 (d, J = 2.5 Hz, 1H), 7.32 (dd, J = 8.2, |
| | | | | | 1.3 Hz, 1H), 6.52 (d, J = 3.0 Hz, |
| | | | | | 1H), 4.80 (dt, J = 13.3, 6.7 Hz, |
| | | | | | 1H), 3.38 (s, 3H), 1.49 (d, J = 6.7 Hz, |
| | | | | | 6H). |
| 50 | 50 | 419.5 | 8-[3- | 97.3% | ¹H NMR (400 MHz, DMSO) δ |
| | | | (dimethylamino)phenyl]- | [M + H]⁺ = 420.2 | 8.99 (s, 1H), 8.80 (d, J = 1.8 Hz, |
| | | | N-(4- | | 1H), 8.71 (d, J = 1.8 Hz, 1H), |
| | | | methanesulfonylpyridin- | | 8.57 (d, J = 5.1 Hz, 1H), 8.45 (s, |
| | | | 3- | | 1H), 7.84 (d, J = 5.1 Hz, 1H), |
| | | | yl)quinoxalin-6- | | 7.78 (d, J = 2.6 Hz, 1H), |
| | | | amine | | 7.54 (d, J = 2.6 Hz, 1H), |
| | | | | | 7.31-7.26 (m, 1H), 6.98-6.95 (m, 1H), |
| | | | | | 6.91 (d, J = 7.6 Hz, 1H), |
| | | | | | 6.80 (dd, J = 8.6, 2.3 Hz, 1H), |
| | | | | | 3.37 (s, 3H), 2.93 (s, 6H). |
| 51 | 51 | 390.46 | N-(4- | 99.6% | ¹H NMR (400 MHz, DMSO) δ |
| | | | methanesulfonylpyridin- | [M + H]⁺ = 391.2 | 8.98 (s, 1H), 8.82 (d, J = 1.8 Hz, |
| | | | 3-yl)-8-(3- | | 1H), 8.72 (d, J = 1.8 Hz, 1H), |
| | | | methylphenyl)quinoxalin- | | 8.57 (d, J = 5.1 Hz, 1H), 8.45 (s, |
| | | | 6-amine | | 1H), 7.84 (d, J = 5.1 Hz, 1H), |
| | | | | | 7.78 (d, J = 2.5 Hz, 1H), |
| | | | | | 7.57 (d, J = 2.5 Hz, 1H), 7.44 (d, J = 9.1 Hz, |
| | | | | | 2H), 7.38 (t, J = 7.5 Hz, |
| | | | | | 1H), 7.26 (d, J = 7.5 Hz, 1H), |
| | | | | | 3.37 (s, 3H), 2.39 (s, 3H). |
| 52 | 52 | 408.46 | N-methyl-3-{[8-(1- | 96.8% | ¹H NMR (400 MHz, DMSO) δ |
| | | | methyl-1H-indol- | [M + H]⁺ = 409.4 | 9.45 (s, 1H), 8.92 (s, 1H), |
| | | | 6-yl)quinoxalin-6- | | 8.79 (d + q, J = 4.5, 1.8 Hz, 2H), |
| | | | yl]amino}pyridine- | | 8.68 (d, J = 1.8 Hz, 1H), 8.31 (d, J = 5.0 Hz, |
| | | | 4-carboxamide | | 1H), 7.72-7.71 (m, |
| | | | | | 1H), 7.70 (d, J = 2.6 Hz, 1H), |
| | | | | | 7.62 (d, J = 8.2 Hz, 1H), |
| | | | | | 7.56 (d, J = 5.0 Hz, 1H), 7.53 (d, J = 2.6 Hz, |
| | | | | | 1H), 7.40 (d, J = 3.0 Hz, |
| | | | | | 1H), 7.34 (dd, J = 8.2, 1.4 Hz, |
| | | | | | 1H), 6.48 (d, J = 3.0 Hz, 1H), |
| | | | | | 3.83 (s, 3H), 2.75 (d, J = 4.6 Hz, |
| | | | | | 3H). |
| 53 | 53 | 422.48 | N,N-dimethyl-3- | 96.8% | ¹H NMR (400 MHz, DMSO) δ |
| | | | {[8-(1-methyl-1H- | [M + H]⁺ = 423.3 | 8.77 (s, 1H), 8.74 (d, J = 1.8 Hz, |
| | | | indol-6- | | 1H), 8.68 (s, 1H), 8.63 (d, J = 1.8 Hz, |
| | | | yl)quinoxalin-6- | | 1H), 8.38 (d, J = 4.9 Hz, |
| | | | yl]amino}pyridine- | | 1H), 7.71-7.69 (m, 1H), |
| | | | 4-carboxamide | | 7.63 (d, J = 2.6 Hz, 1H), 7.62 (d, J = 8.3 Hz, |
| | | | | | 1H), 7.40 (d, J = 3.0 Hz, |
| | | | | | 1H), 7.37 (d, J = 4.9 Hz, 1H), |
| | | | | | 7.30 (dd + d, J = 8.2, 2.72, 1.4 Hz, |
| | | | | | 2H), 6.48 (d, J = 3.0 Hz, |
| | | | | | 1H), 3.82 (s, 3H), 2.83 (d, J = 1.1 Hz, |
| | | | | | 6H). |
| 54 | 54 | 472.51 | 3-{[8-(1-methyl- | 99.5% | ¹H NMR (400 MHz, DMSO) δ |
| | | | 1H-indol-6- | [M + H]⁺ = 473.4 | 11.01 (s, 1H), 9.13 (s, 1H), |
| | | | yl)quinoxalin-6- | | 8.95 (s, 1H), 8.90 (s, 1H), 8.86 (s, |
| | | | yl]amino}-N- | | 1H), 8.75 (d, J = 1.8 Hz, 1H), |
| | | | (pyrimidin-5- | | 8.64 (d, J = 1.8 Hz, 1H), |
| | | | yl)pyridine-4- | | 8.42 (d, J = 4.9 Hz, 1H), 7.69 (d, J = 4.9 Hz, |
| | | | carboxamide | | 1H), 7.66 (d, J = 2.5 Hz, |
| | | | | | 1H), 7.64-7.63 (m, 1H), |
| | | | | | 7.60 (d, J = 8.1 Hz, 1H), 7.46 (d, J = 2.4 Hz, |
| | | | | | 1H), 7.39 (d, J = 3.0 Hz, |
| | | | | | 1H), 7.26 (dd, J = 8.2, 1.4 Hz, |
| | | | | | 1H), 6.47 (d, J = 3.0 Hz, 1H), |
| | | | | | 3.81 (s, 3H). |
| 55 | 55 | 486.53 | 3-{[8-(1-methyl- | 98.1% | ¹H NMR (400 MHz, DMSO) δ |
| | | | 1H-indol-6- | [M + H]⁺ = 487.5 | 9.35 (t, J = 5.0 Hz, 1H), 9.26 (s, |
| | | | yl)quinoxalin-6- | | 1H), 8.94 (s, 1H), 8.85 (s, 1H), |
| | | | yl]amino}-N- | | 8.75 (d, J = 1.6 Hz, 1H), |
| | | | (pyrimidin-5- | | 8.66 (d + s, J = 1.6 Hz, 3H), 8.37 (d, J = 5.0 Hz, |
| | | | ylmethyl)pyridine- | | 1H), 7.71-7.68 (m, |
| | | | 4-carboxamide | | 1H), 7.63 (d + d + d, J = 8.2, 5.0, |
| | | | | | 2.5 Hz, 3H), 7.40 (d, J = 3.0 Hz, |
| | | | | | 1H), 7.35 (d, J = 2.5 Hz, 1H), |
| | | | | | 7.33 (d, J = 8.2 Hz, 1H), |
| | | | | | 6.48 (d, J = 3.0 Hz, 1H), 4.42 (d, J = 5.2 Hz, |
| | | | | | 2H), 3.84 (s, 3H). |
| 56 | 56 | 488.55 | 3-{[8-(1-methyl- | 95.3% | ¹H NMR (400 MHz, DMSO) δ |
| | | | 1H-indol-6- | [M + H]⁺ = 489.5 | 9.39 (s, 1H), 9.14 (t, J = 5.5 Hz, |
| | | | yl)quinoxalin-6- | | 1H), 8.89 (s, 1H), 8.78 (d, J = 1.8 Hz, |
| | | | yl]amino}-N-[(1- | | 1H), 8.67 (d, J = 1.8 Hz, |
| | | | methyl-1H- | | 1H), 8.33 (d, J = 5.0 Hz, 1H), |
| | | | pyrazol-4- | | 7.73-7.70 (m, 1H), 7.69 (d, J = 2.6 Hz, |
| | | | yl)methyl]pyridine- | | 1H), 7.62 (d, J = 8.2 Hz, |
| | | | 4-carboxamide | | 1H), 7.58 (d, J = 5.0 Hz, |
| | | | | | 1H), 7.51 (s, 1H), 7.47 (d, J = 2.6 Hz, |
| | | | | | 1H), 7.40 (d, J = 3.0 Hz, |
| | | | | | 1H), 7.34 (dd, J = 8.1, 1.3 Hz, |
| | | | | | 1H), 7.30 (s, 1H), 6.48 (d, J = 3.0 Hz, |
| | | | | | 1H), 4.24 (d, J = 5.6 Hz, |
| | | | | | 2H), 3.84 (s, 3H), 3.67 (s, 3H). |
| 57 | 57 | 457.55 | 4- | 94.8% | ¹H NMR (400 MHz, DMSO): δ |
| | | | methanesulfonyl- | [M + H]⁺ = 458.2 | 8.80 (d, J = 1.8 Hz, 1H), |
| | | | N1-methyl-N3-[8- | | 8.69 (d, J = 1.8 Hz, 1H), 8.22 (s, 1H), |
| | | | (1-methyl-1H- | | 7.77 (d, J = 2.5 Hz, 1H), 7.71 (s, |
| | | | indol-6- | | 1H), 7.65-7.56 (m, 3H), |
| | | | yl)quinoxalin-6- | | 7.40 (d, J = 3.1 Hz, 1H), 7.34 (dd, J = 8.2, |
| | | | yl]benzene-1,3- | | 1.4 Hz, 1H), 6.77 (dd, J = 9.1, |
| | | | diamine | | 3.6 Hz, 2H), 6.49 (d, J = 3.0 Hz, |
| | | | | | 1H), 6.40 (dd, J = 8.9, 2.1 Hz, |
| | | | | | 1H), 3.83 (s, 3H), 3.10 (s, |
| | | | | | 3H), 2.73 (d, J = 4.9 Hz, 3H). |
| 58 | 58 | 464.92 | 8-[3- | 85.6% | ¹H NMR (400 MHz, |
| | | | (chloromethyl)-1- | [M + H]⁺ = 465.30 | DMSO) δ 10.80 (s, 1H), |
| | | | benzofuran-5-yl]- | | 9.03 (d, J = 1.7 Hz, 1H), 8.91 (d, J = 1.8 Hz, |
| | | | N-(4- | | 1H), 8.89 (s, 1H), |
| | | | methanesulfonylpyridin- | | 8.65 (s, 2H), 8.51 (d, J = 5.1 Hz, 1H), |
| | | | 3- | | 8.43 (s, 1H), 8.01 (s, 1H), |
| | | | yl)quinoxalin-6- | | 7.81 (d, J = 5.0 Hz, 1H), 7.61 (dd, J = 32.2, |
| | | | amine | | 8.5 Hz, 2H), 3.41 (s, 3H), |
| | | | | | 2.23 (s, 2H). |
| 59 | 59 | 447.49 | 8-(7-fluoro-1- | 96.4% | ¹H NMR (400 MHz, |
| | | | methyl-1H-indol- | [M + H]⁺ = 448.2 | DMSO) δ 8.99 (s, 1H), 8.82 (d, |
| | | | 6-yl)-N-(4- | | J = 1.9 Hz, 1H), 8.69 (d, J = 1.9 Hz, |
| | | | methanesulfonylpyridin- | | 1H), 8.58 (d, J = 5.1 Hz, |
| | | | 3- | | 1H), 8.50 (s, 1H), 7.85 (d, J = 5.1 Hz, |
| | | | yl)quinoxalin-6- | | 1H), 7.80 (d, J = 2.6 Hz, |
| | | | amine | | 1H), 7.60 (d, J = 2.6 Hz, 1H), |
| | | | | | 7.43 (d, J = 8.1 Hz, 1H), |
| | | | | | 7.41 (d, J = 3.1 Hz, 1H), 7.06 (dd, J = 8.1, |
| | | | | | 6.1 Hz, 1H), 6.54 (t, J = 2.8 Hz, |
| | | | | | 1H), 3.97 (d, J = 2.0 Hz, |
| | | | | | 3H), 3.38 (s, 3H). |
| 60 | 60 | 404.49 | 8-(4-ethylphenyl)- | 99.9% | ¹H NMR (400 MHz, |
| | | | N-(4- | [M + H]⁺ = 405.2 | DMSO) δ 8.98 (s, 1H), 8.82 (d, |
| | | | methanesulfonylpyridin- | | J = 1.8 Hz, 1H), 8.72 (d, J = 1.8 Hz, |
| | | | 3- | | 1H), 8.57 (d, J = 5.1 Hz, |
| | | | yl)quinoxalin-6- | | 1H), 8.45 (s, 1H), 7.84 (d, J = 5.1 Hz, |
| | | | amine | | 1H), 7.78 (d, J = 2.6 Hz, |
| | | | | | 1H), 7.58 (d, J = 4.8 Hz, 1H), |
| | | | | | 7.58 (d, J = 8.2 Hz, 1H), |
| | | | | | 7.56 (d, J = 2.6 Hz, 1H), 7.34 (d, J = 8.1 Hz, |
| | | | | | 2H), 3.37 (s, 3H), |
| | | | | | 2.69 (q, J = 7.6 Hz, 2H), 1.25 (t, J = 7.6 Hz, |
| | | | | | 3H). |
| 61 | 61 | 416.46 | 8-(1H-1,3- | 97.1% | ¹H NMR (400 MHz, |
| | | | benzodiazol-5-yl)- | [M + H]⁺ = 417.2 | DMSO) δ 12.59 (s, 1H), 9.02 (s, |
| | | | N-(4- | | 1H), 8.83 (d, J = 1.8 Hz, 1H), |
| | | | methanesulfonylpyridin- | | 8.73 (d, J = 1.8 Hz, 1H), |
| | | | 3- | | 8.57 (d, J = 5.1 Hz, 1H), 8.47 (s, 1H), |
| | | | yl)quinoxalin-6- | | 8.30 (s, 1H), 7.90 (s, 1H), |
| | | | amine | | 7.85 (s, 1H), 7.85 (d, J = 7.0 Hz, 1H), |
| | | | | | 7.69 (d, J = 7.5 Hz, 1H), |
| | | | | | 7.58 (d, J = 2.6 Hz, 1H), 7.51 (d, J = 9.0 Hz, |
| | | | | | 1H), 3.38 (s, 3H). |
| 62 | 62 | 406.46 | N-(4- | 98.9% | ¹H NMR (400 MHz, |
| | | | methanesulfonylpyridin- | [M + H]⁺ = 407.2 | DMSO) δ 8.99 (s, 1H), 8.82 (d, |
| | | | 3-yl)-8-(3- | | J = 1.8 Hz, 1H), 8.72 (d, J = 1.8 Hz, |
| | | | methoxyphenyl)quinoxalin- | | 1H), 8.57 (d, J = 5.1 Hz, |
| | | | 6-amine | | 1H), 8.45 (s, 1H), 7.84 (d, J = 5.1 Hz, |
| | | | | | 1H), 7.81 (d, J = 2.6 Hz, |
| | | | | | 1H), 7.57 (d, J = 2.5 Hz, 1H), |
| | | | | | 7.41 (t, J = 8.1 Hz, 1H), |
| | | | | | 7.21 (m, 2H), 7.02 (dd, J = 8.0, 2.3 Hz, |
| | | | | | 1H), 3.81 (s, 3H), 3.37 (s, |
| | | | | | 3H). |
| 63 | 63 | 446.53 | 8-(3,3-dimethyl- | 96.6% | ¹H NMR (400 MHz, |
| | | | 2,3-dihydro-1- | [M + H]⁺ = 447.2 | DMSO) δ 8.99 (s, 1H), 8.80 (d, |
| | | | benzofuran-5-yl)- | | J = 1.8 Hz, 1H), 8.71 (d, J = 1.8 Hz, |
| | | | N-(4- | | 1H), 8.57 (d, J = 5.1 Hz, |
| | | | methanesulfonylpyridin- | | 1H), 8.42 (s, 1H), 7.84 (d, J = 5.1 Hz, |
| | | | 3- | | 1H), 7.78 (d, J = 2.6 Hz, |
| | | | yl)quinoxalin-6- | | 1H), 7.50 (d, J = 2.6 Hz, 1H), |
| | | | amine | | 7.49 (d, J = 1.8 Hz, 1H), |
| | | | | | 7.43 (dd, J = 8.2, 1.9 Hz, 1H), |
| | | | | | 6.89 (d, J = 8.2 Hz, 1H), 4.29 (s, 2H), |
| | | | | | 3.37 (s, 3H), 1.35 (s, 6H). |
| 65 | 65 | 404.49 | 8-(3-ethylphenyl)- | 99.9% | ¹H NMR (400 MHz, |
| | | | N-(4- | [M + H]⁺ = 405.2 | DMSO) δ 9.00 (s, 1H), 8.82 (d, |
| | | | methanesulfonylpyridin- | | J = 1.8 Hz, 1H), 8.72 (d, J = 1.8 Hz, |
| | | | 3- | | 1H), 8.58 (d, J = 5.1 Hz, |
| | | | yl)quinoxalin-6- | | 1H), 8.46 (s, 1H), 7.85 (dd, J = 5.1, |
| | | | amine | | 0.4 Hz, 1H), 7.80 (d, J = 2.6 Hz, |
| | | | | | 1H), 7.56 (d, J = 2.6 Hz, |
| | | | | | 1H), 7.47 (ddd, J = 9.0, 2.0, 1.1 Hz, |
| | | | | | 2H), 7.40 (td, J = 7.5, 0.6 Hz, |
| | | | | | 1H), 7.29 (dt, J = 7.4, 1.4 Hz, |
| | | | | | 1H), 3.38 (s, 3H), 2.69 (q, J = 7.6 Hz, |
| | | | | | 2H), 1.24 (t, J = 7.6 Hz, |
| | | | | | 3H). |
| 66 | 66 | 405.48 | 8-(2-amino-5- | 99.7% | ¹H NMR (400 MHz, |
| | | | methylphenyl)-N- | [M + H]⁺ = 406.2 | DMSO) δ 8.99 (s, 1H), 8.80 (d, |
| | | | (4- | | J = 1.8 Hz, 1H), 8.67 (d, J = 1.9 Hz, |
| | | | methanesulfonylpyridin- | | 1H), 8.53 (d, J = 5.1 Hz, |
| | | | 3- | | 1H), 8.40 (s, 1H), 7.82 (d, J = 5.1 Hz, |
| | | | yl)quinoxalin-6- | | 1H), 7.61 (dd, J = 6.2, |
| | | | amine | | 2.6 Hz, 2H), 6.92 (dd, J = 8.1, |
| | | | | | 1.6 Hz, 1H), 6.83 (d, J = 1.5 Hz, |
| | | | | | 1H), 6.67 (d, J = 8.1 Hz, 1H), |
| | | | | | 4.47 (s, 2H), 3.37 (s, 3H), |
| | | | | | 2.18 (s, 3H). |
| 67 | 67 | 406.46 | 2-{7-[(4- | 98.4% | ¹H NMR (400 MHz, |
| | | | methanesulfonylpyridin- | [M + H]⁺ = 407.2 | DMSO) δ 9.04 (s, 1H), 8.94 (s, |
| | | | 3- | | 1H), 8.78 (d, J = 1.8 Hz, 1H), |
| | | | yl)amino]quinoxalin- | | 8.66 (d, J = 1.8 Hz, 1H), |
| | | | 5-yl}-4- | | 8.54 (d, J = 5.1 Hz, 1H), 8.42 (s, 1H), |
| | | | methylphenol | | 7.83 (d, J = 5.1 Hz, 1H), |
| | | | | | 7.66 (d, J = 2.6 Hz, 1H), 7.57 (d, J = 2.6 Hz, |
| | | | | | 1H), 7.07-7.01 (m, |
| | | | | | 2H), 6.82 (d, J = 8.8 Hz, 1H), |
| | | | | | 3.37 (s, 3H), 2.24 (s, 3H). |
| 68 | 68 | 419.45 | 8-(1-methyl-1H- | 98.1% | ¹H NMR (400 MHz, |
| | | | indol-6-yl)-N-[4- | [M + H]⁺ = 420.4 | DMSO) δ 10.79 (s, 1H), 9.03 (s, |
| | | | (1H-1,2,3,4- | | 1H), 8.81 (d, J = 1.8 Hz, 1H), |
| | | | tetrazol-5- | | 8.69 (d, J = 1.8 Hz, 1H), |
| | | | yl)pyridin-3- | | 8.23 (d, J = 5.0 Hz, 1H), 8.10 (d, J = 5.0 Hz, |
| | | | yl]quinoxalin-6- | | 1H), 7.76 (d + m, J = 2.7 Hz, |
| | | | amine | | 2H), 7.74 (d, J = 2.6 Hz, |
| | | | | | 1H), 7.64 (d, J = 8.1 Hz, 1H), |
| | | | | | 7.40 (d, J = 3.1 Hz, 1H), |
| | | | | | 7.36 (dd, J = 8.2, 1.4 Hz, 1H), |
| | | | | | 6.49 (dd, J = 3.1, 0.7 Hz, 1H), |
| | | | | | 3.85 (s, 3H). |
| 69 | 69 | 385.86 | N-(4- | 91.9% | ¹H NMR (400 MHz, |
| | | | chloropyridin-3- | [M + H]⁺ = 386.6 | DMSO) δ 8.83 (s, 1H), 8.78 (s, |
| | | | yl)-8-(1-methyl- | | 1H), 8.75 (d, J = 1.8 Hz, 1H), |
| | | | 1H-indol-6- | | 8.65 (d, J = 1.9 Hz, 1H), |
| | | | yl)quinoxalin-6- | | 8.33 (d, J = 5.2 Hz, 1H), 7.69 (s, 1H), |
| | | | amine | | 7.68 (s, 1H), 7.67 (d, J = 2.6 Hz, |
| | | | | | 1H), 7.63 (d, J = 8.1 Hz, 1H), |
| | | | | | 7.40 (d, J = 3.1 Hz, 1H), |
| | | | | | 7.31 (dd, J = 8.2, 1.5 Hz, 1H), |
| | | | | | 7.15 (d, J = 2.6 Hz, 1H), 6.48 (dd, J = 3.1, |
| | | | | | 0.8 Hz, 1H), 3.82 (s, 3H). |
| 71 | 71 | 447.49 | 8-(4-fluoro-1- | 96.8% | ¹H NMR (400 MHz, |
| | | | methyl-1H-indol- | [M + H]⁺ = 448.2 | DMSO) δ 9.01 (s, 1H), 8.83 (d, |
| | | | 6-yl)-N-(4- | | J = 1.8 Hz, 1H), 8.75 (d, J = 1.9 Hz, |
| | | | methanesulfonylpyridin- | | 1H), 8.57 (d, J = 5.1 Hz, |
| | | | 3- | | 1H), 8.47 (s, 1H), 7.90 (d, J = 2.6 Hz, |
| | | | yl)quinoxalin-6- | | 1H), 7.85 (d, J = 5.1 Hz, |
| | | | amine | | 1H), 7.60 (s, 1H), 7.58 (d, J = 2.5 Hz, |
| | | | | | 1H), 7.47 (d, J = 3.1 Hz, |
| | | | | | 1H), 7.17 (7.17 (dd, J = 11.8, |
| | | | | | 1.2 Hz, 1H)), 6.56 (6.56 (dd, J = 3.1, |
| | | | | | 0.8 Hz, 1H)), 3.86 (s, 3H), |
| | | | | | 3.39 (s, 3H). |
| 72 | 72 | 446.48 | 4- | 95.8% | ¹H NMR (400 MHz, |
| | | | methanesulfonyl- | [M + H]⁺ = 447.1 | DMSO) δ 8.89 (d, J = 1.8 Hz, |
| | | | 3-{[8-(3-methyl-1- | | 1H), 8.81 (d, J = 1.8 Hz, 1H), |
| | | | benzofuran-5- | | 8.45 (s, 1H), 8.38 (d, J = 1.5 Hz, |
| | | | yl)quinoxalin-6- | | 1H), 8.03 (dd, J = 6.8, 1.7 Hz, |
| | | | yl]amino}pyridin- | | 1H), 7.94 (d, J = 2.5 Hz, 1H), |
| | | | 1-ium-1-olate | | 7.87 (d, J = 2.5 Hz, 1H), |
| | | | | | 7.86 (dd, J = 1.8, 0.7 Hz, 1H), |
| | | | | | 7.85 (d, J = 1.4 Hz, 1H),, 7.77 (d, J = 6.8 Hz, |
| | | | | | 1H), 7.66 (d, J = 8.5 Hz, |
| | | | | | 1H), 7.61 (dd, J = 8.5, 1.6 Hz, |
| | | | | | 1H), 3.38 (s, 3H), 2.26 (d, J = 1.1 Hz, |
| | | | | | 3H). |
| 73 | 73 | 447.49 | 8-(5-fluoro-1- | 95.8% | ¹H NMR (400 MHz, |
| | | | methyl-1H-indol- | [M + H]⁺ = 448.2 | DMSO) δ 9.01 (s, 1H), 8.82 (d, |
| | | | 6-yl)-N-(4- | | J = 1.8 Hz, 1H), 8.68 (d, J = 1.8 Hz, |
| | | | methanesulfonylpyridin-3- | | 1H), 8.58 (d, J = 5.1 Hz, |
| | | | yl)quinoxalin-6- | | 1H), 8.50 (s, 1H), 7.85 (dd, J = 5.1, |
| | | | amine | | 0.4 Hz, 1H), 7.83 (d, J = 2.6 Hz, |
| | | | | | 1H), 7.60 (d, J = 2.6 Hz, |
| | | | | | 1H), 7.56 (d, J = 6.1 Hz, 1H), |
| | | | | | 7.46 (d, J = 3.1 Hz, 1H), |
| | | | | | 7.42 (d, J = 10.6 Hz, 1H), 6.49 (dd, J = 3.0, |
| | | | | | 0.8 Hz, 1H), 3.82 (s, 3H), |
| | | | | | 3.39 (s, 3H). |
| 74 | 74 | 420.49 | N-(4- | 98.5% | ¹H NMR (400 MHz, |
| | | | methanesulfonylpyridin- | [M + H]⁺ = 421.2 | DMSO) δ 8.95 (s, 1H), 8.78 (d, |
| | | | 3-yl)-8-(2- | | J = 1.9 Hz, 1H), 8.65 (d, J = 1.8 Hz, |
| | | | methoxy-5- | | 1H), 8.55 (d, J = 5.1 Hz, |
| | | | methylphenyl)quinoxalin- | | 1H), 8.42 (s, 1H), 7.83 (dd, J = 5.1, |
| | | | 6-amine | | 0.6 Hz, 1H), 7.65 (d, J = 2.6 Hz, |
| | | | | | 1H), 7.58 (d, J = 2.6 Hz, |
| | | | | | 1H), 7.22 (ddd, J = 8.5, 2.2, 0.5 Hz, |
| | | | | | 1H), 7.11 (d, J = 2.1 Hz, |
| | | | | | 1H), 7.02 (d, J = 8.4 Hz, 1H), |
| | | | | | 3.62 (s, 3H), 3.37 (s, 3H), |
| | | | | | 2.29 (s, 3H). |
| 75 | 75 | 405.48 | 8-(3-amino-4- | 96.9% | ¹H NMR (400 MHz, |
| | | | methylphenyl)-N- | [M + H]⁺ = 406.1 | DMSO) δ 8.97 (s, 1H), 8.79 (s, |
| | | | (4- | | 1H), 8.71 (s, 1H), 8.57 (d, J = 4.7 Hz, |
| | | | methanesulfonylpyridin- | | 1H), 8.45 (s, 1H), |
| | | | 3- | | 7.85 (d, J = 4.5 Hz, 1H), 7.69 (s, 1H), |
| | | | yl)quinoxalin-6- | | 7.51 (s, 1H), 7.02 (d, J = 7.3 Hz, |
| | | | amine | | 1H), 6.90 (s, 1H), 6.73 (d, J = 7.5 Hz, |
| | | | | | 1H), 4.91 (s, 2H), |
| | | | | | 3.37 (s, 3H), 2.13 (s, 3H). |
| 76 | 76 | 433.53 | 8-[2- | 99.9% | ¹H NMR (400 MHz, |
| | | | (dimethylamino)- | [M + H]⁺ = 434.2 | DMSO) δ 8.85 (s, 1H), 8.80 (d, |
| | | | 5-methylphenyl]- | | J = 1.8 Hz, 1H), 8.68 (d, J = 1.8 Hz, |
| | | | N-(4- | | 1H), 8.52 (d, J = 5.1 Hz, |
| | | | methanesulfonylpyridin- | | 1H), 8.44 (s, 1H), 7.81 (dd, J = 5.1, |
| | | | 3- | | 0.4 Hz, 1H), 7.70 (d, J = 2.6 Hz, |
| | | | yl)quinoxalin-6- | | 1H), 7.65 (d, J = 2.6 Hz, |
| | | | amine | | 1H), 7.14 (ddd, J = 8.2, 2.1, 0.7 Hz, |
| | | | | | 1H), 7.06 (d, J = 8.2 Hz, |
| | | | | | 1H), 7.01 (d, J = 1.8 Hz, 1H), |
| | | | | | 3.36 (s, 3H), 2.37 (s, 6H), |
| | | | | | 2.26 (s, 3H). |
| 81 | 81 | 429.50 | N-(3- | 94.6% | ¹H NMR (400 MHz, |
| | | | methanesulfonylpyridin- | [M + H]⁺ = 430.1 | DMSO) δ 9.24 (s, 1H), 8.88 (d, |
| | | | 2-yl)-8-(1- | | J = 1.8 Hz, 1H), 8.79 (d, J = 1.8 Hz, |
| | | | methyl-1H-indol- | | 1H), 8.66 (d, J = 2.5 Hz, |
| | | | 6-yl)quinoxalin-6- | | 1H), 8.62 (dd, J = 4.8, 1.8 Hz, |
| | | | amine | | 1H), 8.24 (dd, J = 7.8, 1.9 Hz, |
| | | | | | 1H), 7.95 (d, J = 2.5 Hz, 1H), |
| | | | | | 7.74 (s, 1H), 7.65 (dd, J = 8.2, |
| | | | | | 0.7 Hz, 1H), 7.41 (d, J = 3.1 Hz, |
| | | | | | 1H), 7.36 (dd, J = 8.2, 1.4 Hz, |
| | | | | | 1H), 7.19 (dd, J = 7.8, 4.8 Hz, |
| | | | | | 1H), 6.50 (dd, J = 3.1, 0.7 Hz, |
| | | | | | 1H), 3.84 (s, 3H), 3.44 (s, 3H). |
| 82 | 82 | 477.57 | 1-[4-(3-{[8-(1- | 93.1% | ¹H NMR (400 MHz, |
| | | | methyl-1H-indol- | [M + H]⁺ = 478.2 | DMSO) δ 8.71 (d, J = 1.8 Hz, |
| | | | 6-yl)quinoxalin-6- | | 1H), 8.59 (d, J = 1.9 Hz, 2H), |
| | | | yl]amino}pyridin- | | 8.41 (s, 1H), 8.24 (d, J = 5.5 Hz, |
| | | | 4-yl)piperazin-1- | | 1H), 7.67 (s, 1H), 7.63 (d, J = 8.2 Hz, |
| | | | yl]ethan-1-one | | 1H), 7.55 (d, J = 2.6 Hz, |
| | | | | | 1H), 7.40 (d, J = 3.1 Hz, 1H), |
| | | | | | 7.29 (dd, J = 8.1, 1.4 Hz, 1H), |
| | | | | | 7.02 (d, J = 5.5 Hz, 1H), |
| | | | | | 6.99 (d, J = 2.6 Hz, 1H), 6.49 (d, J = 3.1 Hz, |
| | | | | | 1H), 3.83 (s, 3H), |
| | | | | | 3.29 (d, J = 11.3 Hz, 4H), 3.14 (d, J = 24.2 Hz, |
| | | | | | 4H), 1.93 (s, 3H). |
| 83 | 83 | 431.50 | N-[4-(1-methyl- | 95.7% | ¹H NMR (400 MHz, |
| | | | 1H-imidazol-4- | [M + H]⁺ = 432.2 | DMSO) δ 9.86 (s, 1H), 8.80 (s, |
| | | | yl)pyridin-3-yl]-8- | | 1H), 8.74 (d, J = 1.8 Hz, 1H), |
| | | | (1-methyl-1H- | | 8.62 (d, J = 1.8 Hz, 1H), |
| | | | indol-6- | | 8.30 (d, J = 5.1 Hz, 1H), 7.86 (s, 2H), |
| | | | yl)quinoxalin-6- | | 7.85 (s, 1H), 7.71 (s, 1H), |
| | | | amine | | 7.66-7.64 (m, 1H), 7.63 (d, J = 3.9 Hz, |
| | | | | | 1H), 7.41 (d, J = 3.0 Hz, |
| | | | | | 1H), 7.33 (dd, J = 8.2, 1.3 Hz, |
| | | | | | 1H), 7.28 (d, J = 2.5 Hz, 1H), |
| | | | | | 6.50 (dd, J = 3.1, 0.9 Hz, 1H), |
| | | | | | 3.84 (s, 3H), 3.71 (s, 3H). |
| 84 | 84 | 408.47 | 8-(1-methyl-1H- | 96.2% | ¹H NMR (400 MHz, |
| | | | indol-6-yl)-N- | [M + H]⁺ = 409.2 | DMSO) δ 8.69 (d, J = 1.8 Hz, |
| | | | {2H,3H,4H- | | 1H), 8.56 (d, J = 1.8 Hz, 1H), |
| | | | pyrido[4,3- | | 8.35 (s, 1H), 7.86 (s, 1H), |
| | | | b][1,4]oxazin-8- | | 7.72 (s, 1H), 7.66 (s, 1H), 7.62 (dd, J = 8.2, |
| | | | yl}quinoxalin-6- | | 0.7 Hz, 1H), 7.59 (d, J = 2.6 Hz, |
| | | | amine | | 1H), 7.40 (d, J = 3.1 Hz, |
| | | | | | 1H), 7.29 (dd, J = 8.2, 1.4 Hz, |
| | | | | | 1H), 7.07 (d, J = 2.6 Hz, 1H), |
| | | | | | 6.49 (dd, J = 3.0, 0.6 Hz, 1H), |
| | | | | | 6.11 (s, 1H), 4.27 (t, J = 4.3 Hz, |
| | | | | | 2H), 3.83 (s, 3H), |
| | | | | | 3.39-3.34 (m, 2H). |
| 85 | 85 | 521.60 | 2-{[8-(1-methyl- | 96.1% | ¹H NMR (400 MHz, |
| | | | 1H-indol-6- | [M + H]⁺ = 522.2 | DMSO) δ 8.92 (s, 1H), 8.83 (d, |
| | | | yl)quinoxalin-6- | | J = 1.8 Hz, 1H), 8.73 (d, J = 1.8 Hz, |
| | | | yl]amino}-N- | | 1H), 8.64 (s, 1H), 8.54 (s, |
| | | | [(pyrimidin-5- | | 2H), 8.04 (s, 1H), 7.81 (dd, J = 7.9, |
| | | | yl)methyl]benzene- | | 1.4 Hz, 1H), 7.74 (s, 1H), |
| | | | 1-sulfonamide | | 7.72 (d, J = 2.6 Hz, 1H), 7.66 (s, |
| | | | | | 1H), 7.64 (s, 1H), 7.57 (d, J = 2.4 Hz, |
| | | | | | 1H), 7.55-7.52 (m, |
| | | | | | 1H), 7.41 (d, J = 3.1 Hz, 1H), |
| | | | | | 7.38 (dd, J = 8.2, 1.4 Hz, 1H), |
| | | | | | 7.13-7.08 (m, 1H), 6.50 (d, J = 3.0 Hz, |
| | | | | | 1H), 4.20 (s, 2H), |
| | | | | | 3.85 (s, 3H). |
| 88 | 88 | 375.44 | 2-{[8-(1-methyl- | 95.4% | ¹H NMR (400 MHz, |
| | | | 1H-indol-6- | [M + H]⁺ = 376.2 | DMSO) δ 9.24 (s, 1H), 8.78 (d, |
| | | | yl)quinoxalin-6- | | J = 1.8 Hz, 1H), 8.69 (d, J = 1.8 Hz, |
| | | | yl]amino}benzonitrile | | 1H), 7.85 (ddd, J = 7.8, 1.6, |
| | | | | | 0.5 Hz, 1H), 7.74 (d, J = 2.6 Hz, |
| | | | | | 1H), 7.72 (dt, J = 1.5, 0.8 Hz, |
| | | | | | 1H), 7.68 (dd, J = 7.0, 1.5 Hz, |
| | | | | | 1H), 7.65 (dd, J = 8.4, 1.3 Hz, |
| | | | | | 1H), 7.64 (dd, J = 8.2, 0.6 Hz, |
| | | | | | 1H), 7.43 (d, J = 2.6 Hz, 1H), |
| | | | | | 7.40 (d, J = 3.1 Hz, 1H), |
| | | | | | 7.33 (dd, J = 8.2, 1.4 Hz, 1H), |
| | | | | | 7.23 (ddd, J = 7.7, 7.1, 1.3 Hz, 1H), |
| | | | | | 6.49 (dd, J = 3.1, 0.7 Hz, 1H), |
| | | | | | 3.83 (s, 3H). |
| 89 | 89 | 393.45 | 2-{[8-(1-methyl- | 95.3% | ¹H NMR (400 MHz, |
| | | | 1H-indol-6- | [M + H]⁺ = 394.1 | DMSO) δ 10.29 (s, 1H), |
| | | | yl)quinoxalin-6- | | 8.78 (d, J = 1.8 Hz, 1H), 8.67 (d, J = 1.8 Hz, |
| | | | yl]amino}benzamide | | 1H), 8.14 (s, 1H), |
| | | | | | 7.77 (dd, J = 7.9, 1.4 Hz, 1H), |
| | | | | | 7.73 (dt, J = 1.5, 0.8 Hz, 1H), |
| | | | | | 7.68 (dd, J = 7.7 Hz, 1H), 7.66 (d, J = 2.6 Hz, |
| | | | | | 1H), 7.62 (dd, J = 8.2, |
| | | | | | 0.6 Hz, 1H), 7.62 (d, J = 2.6 Hz, |
| | | | | | 1H), 7.60 (s, 1H), 7.50 (ddd, J = 8.4, |
| | | | | | 7.3, 1.5 Hz, 1H), 7.39 (d, J = 3.1 Hz, |
| | | | | | 1H), 7.35 (dd, J = 8.2, |
| | | | | | 1.4 Hz, 1H), 7.04 (ddd, J = 8.3, |
| | | | | | 7.6, 1.1 Hz, 1H), 6.48 (dd, J = 3.0, 0.7 Hz, |
| | | | | | 1H), 3.84 (s, 3H). |
| 90 | 90 | 392.42 | 4-cyano-3-{[8-(1- | 99.1% | ¹H NMR (400 MHz, |
| | | | methyl-1H-indol- | [M + H]⁺ = 393.1 | DMSO) δ 9.50 (s, 1H), 8.86 (d, |
| | | | 6-yl)quinoxalin-6- | | J = 1.8 Hz, 1H), 8.78 (d, J = 1.8 Hz, |
| | | | yl]amino}pyridin- | | 1H), 8.33 (d, J = 1.6 Hz, |
| | | | 1-ium-1-olate | | 1H), 7.98 (dd, J = 6.8, 1.7 Hz, |
| | | | | | 1H), 7.87 (dd, J = 6.8, 0.5 Hz, |
| | | | | | 1H), 7.82 (d, J = 2.5 Hz, 1H), |
| | | | | | 7.72 (p, J = 0.7 Hz, 1H), |
| | | | | | 7.69 (d, J = 2.5 Hz, 1H), 7.64 (dd, J = 8.2, |
| | | | | | 0.6 Hz, 1H), 7.41 (d, J = 3.1 Hz, |
| | | | | | 1H), 7.34 (dd, J = 8.2, 1.4 Hz, |
| | | | | | 1H), 6.50 (dd, J = 3.1, 0.9 Hz, |
| | | | | | 1H), 3.83 (s, 3H). |
| 91 | 91 | 390.45 | 3-{methyl[8-(1- | 98.7% | ¹H NMR (400 MHz, |
| | | | methyl-1H-indol- | [M + H]⁺ = 391.2 | DMSO) δ 8.92 (s, 1H), 8.82 (d, |
| | | | 6-yl)quinoxalin-6- | | J = 1.8 Hz, 1H), 8.71 (d, J = 1.8 Hz, |
| | | | yl]amino}pyridine- | | 1H), 8.66 (d, J = 5.0 Hz, |
| | | | 4-carbonitrile | | 1H), 7.97 (dd, J = 5.0, 0.7 Hz, |
| | | | | | 1H), 7.63-7.60 (m, 1H), |
| | | | | | 7.59 (d, J = 8.1 Hz, 1H), 7.38 (d + d, J = 2.9, |
| | | | | | 2.5 Hz, 2H), 7.32 (d, J = 2.8 Hz, |
| | | | | | 1H), 7.22 (dd, J = 8.2, |
| | | | | | 1.4 Hz, 1H), 6.47 (dd, J = 3.1, |
| | | | | | 0.8 Hz, 1H), 3.79 (s, 3H), |
| | | | | | 3.62 (s, 3H). |
| 92 | 92 | 474.53 | 3-{[8-(1-methyl- | 99.1% | ¹H NMR (400 MHz, |
| | | | 1H-indol-6- | [M + H]⁺ = 475.2 | DMSO) δ 10.77 (s, 1H), 9.28 (s, |
| | | | yl)quinoxalin-6- | | 1H), 8.94 (s, 1H), 8.77 (d, J = 1.8 Hz, |
| | | | yl]amino}-N-(1- | | 1H), 8.67 (d, J = 1.9 Hz, |
| | | | methyl-1H- | | 1H), 8.38 (d, J = 5.0 Hz, 1H), |
| | | | pyrazol-4- | | 7.96 (s, 1H), 7.71 (d, J = 2.6 Hz, |
| | | | yl)pyridine-4- | | 1H), 7.70-7.69 (m, 1H), |
| | | | carboxamide | | 7.67 (d, J = 4.9 Hz, 1H), 7.62 (d, J = 8.1 Hz, |
| | | | | | 1H), 7.52 (m, 2H), |
| | | | | | 7.40 (d, J = 3.0 Hz, 1H), 7.31 (dd, J = 8.2, |
| | | | | | 1.4 Hz, 1H), 6.48 (dd, J = 3.0, |
| | | | | | 0.7 Hz, 1H), 3.83 (s, 3H), |
| | | | | | 3.78 (s, 3H). |
| 93 | 93 | 508.60 | N-[2- | 94.8% | ¹H NMR (400 MHz, |
| | | | methanesulfonyl- | [M + H]⁺ = 509.2 | DMSO) δ 8.81 (d, J = 1.8 Hz, |
| | | | 5-(1-methyl-1H- | | 1H), 8.72 (d, J = 1.8 Hz, 1H), |
| | | | pyrazol-5- | | 8.48 (s, 1H), 8.02 (d, J = 8.3 Hz, |
| | | | yl)phenyl]-8-(1- | | 1H), 7.89 (d, J = 2.6 Hz, 1H), |
| | | | methyl-1H-indol- | | 7.83 (d, J = 1.5 Hz, 1H), |
| | | | 6-yl)quinoxalin-6- | | 7.69 (dt, J = 1.6, 0.7 Hz, 1H), |
| | | | amine | | 7.66 (d, J = 2.6 Hz, 1H), 7.63 (dd, J = 8.2, |
| | | | | | 0.7 Hz, 1H), 7.49 (d, J = 2.0 Hz, |
| | | | | | 1H), 7.47 (dd, J = 8.3, 1.7 Hz, |
| | | | | | 1H), 7.41 (d, J = 3.0 Hz, |
| | | | | | 1H), 7.35 (dd, J = 8.2, 1.4 Hz, |
| | | | | | 1H), 6.53 (d, J = 1.9 Hz, 1H), |
| | | | | | 6.49 (dd, J = 3.0, 0.6 Hz, 1H), |
| | | | | | 3.88 (s, 3H), 3.81 (s, 3H), |
| | | | | | 3.34 (s, 3H). |
| 94 | 94 | 495.56 | N-[2- | 94.8% | ¹H NMR (400 MHz, |
| | | | methanesulfonyl- | [M + H]⁺ = 496.3 | DMSO) δ 8.84 (d, J = 1.7 Hz, |
| | | | 5-(1,3-oxazol-2- | | 1H), 8.77 (d, J = 1.7 Hz, 1H), |
| | | | yl)phenyl]-8-(1- | | 8.50 (s, 1H), 8.32 (d, J = 0.8 Hz, |
| | | | methyl-1H-indol- | | 1H), 8.26 (d, J = 1.1 Hz, 1H), |
| | | | 6-yl)quinoxalin-6- | | 8.07 (d, J = 8.3 Hz, 1H), |
| | | | amine | | 7.92 (d, J = 2.5 Hz, 1H), 7.83 (dd, J = 8.3, |
| | | | | | 1.3 Hz, 1H), 7.74 (d, J = 2.5 Hz, |
| | | | | | 1H), 7.71 (dd, J = 1.6, 0.8 Hz, |
| | | | | | 1H), 7.62 (d, J = 8.2 Hz, |
| | | | | | 1H), 7.46 (d, J = 0.8 Hz, 1H), |
| | | | | | 7.39 (dd, J = 9.3, 2.1 Hz, 2H), |
| | | | | | 6.48 (d, J = 3.0 Hz, 1H), 3.79 (s, |
| | | | | | 3H), 3.35 (s, 3H). |
| 95 | 95 | 408.47 | 3-{methyl[8-(1- | 97.8% | ¹H NMR (400 MHz, |
| | | | methyl-1H-indol- | [M + H]⁺ = 409.2 | DMSO) δ 8.75 (d, J = 1.8 Hz, |
| | | | 6-yl)quinoxalin-6- | | 1H), 8.66 (s, 1H), 8.61 (d, J = 4.9 Hz, |
| | | | yl]amino}pyridine- | | 1H), 8.59 (d, J = 1.9 Hz, |
| | | | 4-carboxamide | | 1H), 7.99 (s, 1H), 7.63 (s, 1H), |
| | | | | | 7.58-7.57 (m, 1H), 7.57 (dd, J = 3.2, |
| | | | | | 0.7 Hz, 1H), 7.55 (d, J = 0.7 Hz, |
| | | | | | 1H), 7.36 (d, J = 3.0 Hz, |
| | | | | | 1H), 7.19 (dd, J = 8.1, 1.4 Hz, |
| | | | | | 1H), 7.13 (d, J = 2.8 Hz, 1H), |
| | | | | | 7.10 (d, J = 2.8 Hz, 1H), |
| | | | | | 6.44 (dd, J = 3.0, 0.7 Hz, 1H), |
| | | | | | 3.78 (s, 3H), 3.46 (s, 3H). |
| 96 | 96 | 470.54 | 3-{[8-(1-methyl- | 92.9% | ¹H NMR (400 MHz, |
| | | | 1H-indol-6- | [M + H]⁺ = 471.2 | DMSO) δ 10.59 (s, 1H), 9.03 (s, |
| | | | yl)quinoxalin-6- | | 1H), 8.89 (s, 1H), 8.75 (d, J = 1.8 Hz, |
| | | | yl]amino}-N- | | 1H), 8.63 (d, J = 1.9 Hz, |
| | | | phenylpyridine-4- | | 1H), 8.42 (d, J = 4.9 Hz, 1H), |
| | | | carboxamide | | 7.70 (d, J = 2.6 Hz, 1H), |
| | | | | | 7.68 (d, J = 4.9 Hz, 1H), |
| | | | | | 7.66-7.64 (m, 1H), 7.61 (dd, J = 8.2, 0.7 Hz, |
| | | | | | 1H), 7.63-7.60 (m, 2H), |
| | | | | | 7.45 (d, J = 2.5 Hz, 1H), |
| | | | | | 7.39 (d, J = 3.0 Hz, 1H), |
| | | | | | 7.28 (d + d + dd, J = 8.1, 7.4, 1.4 Hz, |
| | | | | | 3H), 7.07 (t, J = 7.4 Hz, 1H), |
| | | | | | 6.47 (d, J = 3.0 Hz, 1H), 3.81 (s, |
| | | | | | 3H). |
| 97 | 97 | 505.58 | 3-{[8-(1-methyl- | 97.5% | ¹H NMR (400 MHz, |
| | | | 1H-indol-6- | [M + H]⁺ = 506.2 | DMSO) δ 9.22 (s, 1H), 8.85 (s, |
| | | | yl)quinoxalin-6- | | 1H), 8.80 (d, J = 7.2 Hz, 1H), |
| | | | yl]amino}-N-(1- | | 8.77 (d, J = 1.8 Hz, 1H), |
| | | | methyl-2- | | 8.66 (d, J = 1.8 Hz, 1H), 8.36 (d, J = 5.0 Hz, |
| | | | oxopiperidin-4- | | 1H), 7.72-7.70 (m, |
| | | | yl)pyridine-4- | | 1H), 7.66 (d, J = 2.6 Hz, 1H), |
| | | | carboxamide | | 7.63 (dd, J = 8.2, 0.7 Hz, 1H), |
| | | | | | 7.58 (dd, J = 5.0, 0.6 Hz, 1H), |
| | | | | | 7.42 (d, J = 2.6 Hz, 1H), |
| | | | | | 7.40 (d, J = 3.0 Hz, 1H), 7.33 (dd, J = 8.2, |
| | | | | | 1.4 Hz, 1H), 6.48 (dd, J = 3.0, |
| | | | | | 0.6 Hz, 1H), 4.11 (m, 1H), |
| | | | | | 3.83 (s, 3H), 3.19 (m, 2H), |
| | | | | | 2.74 (s, 3H), 2.41 (dd, J = 17.0, 5.3 Hz, |
| | | | | | 1H), 2.17 (dd, J = 17.2, 8.6 Hz, |
| | | | | | 1H), 1.93-1.84 (m, 1H), |
| | | | | | 1.74-1.63 (m, Hz, 1H). |
| 98 | 98 | 491.56 | N-(1- | 95.7% | ¹H NMR (400 MHz, |
| | | | acetylazetidin-3- | [M + H]⁺ = 492.2 | DMSO) δ 9.29 (d, J = 6.7 Hz, |
| | | | yl)-3-{[8-(1- | | 1H), 9.27 (s, 1H), 8.86 (s, 1H), |
| | | | methyl-1H-indol- | | 8.77 (d, J = 1.8 Hz, 1H), |
| | | | 6-yl)quinoxalin-6- | | 8.67 (d, J = 1.8 Hz, 1H), 8.36 (d, J = 5.0 Hz, |
| | | | yl]amino}pyridine- | | 1H), 7.71-7.69 (m, |
| | | | 4-carboxamide | | 1H), 7.66 (d, J = 2.6 Hz, 1H), |
| | | | | | 7.61 (d + d, J = 8.1, 4.9 Hz, 2H), |
| | | | | | 7.44 (d, J = 2.6 Hz, 1H), |
| | | | | | 7.40 (d, J = 3.0 Hz, 1H), 7.32 (dd, J = 8.2, |
| | | | | | 1.4 Hz, 1H), 6.49 (dd, J = 3.1, |
| | | | | | 0.8 Hz, 1H), |
| | | | | | 4.61-4.48 (m, 2H), 4.25 (t, J = 8.3 Hz, |
| | | | | | 1H), 3.99 (t, J = 8.7 Hz, 1H), |
| | | | | | 3.83 (s + dd, J = 9.8, 5.3 Hz, 4H), |
| | | | | | 3.72 (dd, J = 9.8, 5.3 Hz, 1H), |
| | | | | | 1.64 (s, 3H). |
| 99 | 99 | 477.57 | 3-{[8-(1-methyl- | 93.7% | ¹H NMR (400 MHz, |
| | | | 1H-indol-6- | [M + H]⁺ = 478.2 | DMSO) δ 9.27 (s, 1H), 8.84 (s, |
| | | | yl)quinoxalin-6- | | 1H), 8.78 (d, J = 8.3 Hz, 1H), |
| | | | yl]amino}-N-(1- | | 8.77 (d, J = 1.9 Hz, 1H), |
| | | | methylpyrrolidin- | | 8.66 (d, J = 1.8 Hz, 1H), 8.34 (d, J = 5.0 Hz, |
| | | | 3-yl)pyridine-4- | | 1H), 7.72-7.69 (m, |
| | | | carboxamide | | 1H), 7.65 (d, J = 2.6 Hz, 1H), |
| | | | | | 7.62 (d, J = 8.2 Hz, 1H), |
| | | | | | 7.58 (d, J = 5.0 Hz, 1H), 7.41 (d, J = 2.6 Hz, |
| | | | | | 1H), 7.40 (d, J = 3.1 Hz, |
| | | | | | 1H), 7.33 (dd, J = 8.2, 1.4 Hz, |
| | | | | | 1H), 6.49 (dd, J = 3.1, 0.8 Hz, |
| | | | | | 1H), 4.27 (dddd, J = 11.7, 9.3, |
| | | | | | 7.0, 4.8 Hz, 1H), 3.83 (s, 3H), |
| | | | | | 2.53 (dd, J = 8.3, 5.9 Hz, 1H), |
| | | | | | 2.44 (td, J = 8.4, 5.6 Hz, 1H), |
| | | | | | 2.29 (td, J = 8.3, 6.1 Hz, 1H), |
| | | | | | 2.21 (dd, J = 9.4, 4.8 Hz, 1H), |
| | | | | | 2.12 (s, 3H), 2.08-1.97 (m, |
| | | | | | 1H), 1.61 (ddt, J = 13.0, 8.2, |
| | | | | | 5.7 Hz, 1H). |
| 100 | 100 | 507.57 | 2-{[8-(1-methyl- | 93.8% | ¹H NMR (400 MHz, DMSO-d6) |
| | | | 1H-indol-6- | [M + H]⁺ = 508.1 | δ 10.99-10.93 (m, 1H), |
| | | | yl)quinoxalin-6- | | 8.79 (d, J = 1.8 Hz, 1H), 8.70 (d, J = 1.8 Hz, |
| | | | yl]amino}-N- | | 1H), 8.66 (s, 1H), |
| | | | (pyrimidin-5- | | 8.45 (s, 2H), 8.07 (s, 1H), 7.98 (dd, J = 7.9, |
| | | | yl)benzene-1- | | 1.3 Hz, 1H), |
| | | | sulfonamide | | 7.70-7.68 (m, 1H), 7.66 (d, J = 1.2 Hz, |
| | | | | | 1H), 7.65 (s, 1H), 7.63 (s, 1H), |
| | | | | | 7.55 (d, J = 2.5 Hz, 1H), |
| | | | | | 7.42 (d, J = 3.1 Hz, 1H), |
| | | | | | 7.35-7.31 (m, 2H), 7.25 (ddd, J = 8.3, 7.1, |
| | | | | | 1.5 Hz, 1H), 6.50 (dd, J = 3.1, |
| | | | | | 0.8 Hz, 1H), 3.86 (s, 3H). |
| 101 | 101 | 478.56 | 3-{[8-(1-methyl- | 93.4% | ¹H NMR (400 MHz, |
| | | | 1H-indol-6- | [M + H]⁺ = 479.2 | DMSO) δ 9.29 (s, 1H), 8.87 (s, |
| | | | yl)quinoxalin-6- | | 1H), 8.78 (d, J = 1.7 Hz, 1H), |
| | | | yl]amino}-N- | | 8.73-8.61 (m, 2H), 8.36 (d, J = 4.9 Hz, |
| | | | (oxan-4- | | 1H), 7.71 (s, 1H), |
| | | | yl)pyridine-4- | | 7.67 (d, J = 2.5 Hz, 1H), 7.63 (d, J = 8.2 Hz, |
| | | | carboxamide | | 1H), 7.58 (d, J = 4.9 Hz, |
| | | | | | 1H), 7.44 (d, J = 2.5 Hz, 1H), |
| | | | | | 7.41 (d, J = 3.0 Hz, 1H), |
| | | | | | 7.33 (dd, J = 8.2, 1.5 Hz, 1H), |
| | | | | | 6.49 (d, J = 2.8 Hz, 1H), |
| | | | | | 3.95-3.86 (m, 1H), 3.84 (s, 3H), |
| | | | | | 3.82-3.71 (m, 2H), 3.31-3.25 (m, |
| | | | | | 2H), 1.73-1.58 (m, 2H), |
| | | | | | 1.44 (qd, J = 12.4, 11.9, 4.3 Hz, 2H). |
| 102 | 102 | 444.51 | 6- | 92.2% | ¹H NMR (400 MHz, |
| | | | methanesulfonyl- | [M + H]⁺ = 445.2 | DMSO) δ 8.82 (d, J = 1.8 Hz, |
| | | | N1-[8-(3-methyl- | | 1H), 8.71 (d, J = 1.8 Hz, 1H), |
| | | | 1-benzofuran-5- | | 8.19 (s, 1H), 7.87 (d, J = 0.9 Hz, |
| | | | yl)quinoxalin-6- | | 1H), 7.84 (d, J = 1.3 Hz, 1H), |
| | | | yl]benzene-1,3- | | 7.77 (d, J = 2.5 Hz, 1H), |
| | | | diamine | | 7.66-7.63 (m, 1H), 7.63 (d, J = 1.7 Hz, |
| | | | | | 1H), 7.61 (d, J = 2.7 Hz, |
| | | | | | 1H), 7.53 (d, J = 8.7 Hz, 1H), |
| | | | | | 6.83 (d, J = 2.0 Hz, 1H), |
| | | | | | 6.39 (dd, J = 8.7, 2.0 Hz, 1H), |
| | | | | | 6.20 (s, 2H), 3.10 (s, 3H), 2.26 (d, J = 1.0 Hz, |
| | | | | | 3H). |
| 103 | 103 | 474.49 | N-(2- | 93.2% | ¹H NMR (400 MHz, |
| | | | methanesulfonyl- | [M + H]⁺ = 475.1 | DMSO) δ 8.89 (d, J = 1.8 Hz, |
| | | | 5-nitrophenyl)-8- | | 1H), 8.82 (dd, J = 1.9, 0.5 Hz, |
| | | | (3-methyl-1- | | 1H), 8.66 (s, 1H), 8.35 (d, J = 2.2 Hz, |
| | | | benzofuran-5- | | 1H), 8.16 (d, J = 8.7 Hz, |
| | | | yl)quinoxalin-6- | | 1H), 7.97 (d, J = 2.5 Hz, 2H), |
| | | | amine | | 7.97 (dd, J = 8.7, 2.2 Hz, 1H), |
| | | | | | 7.89 (d, J = 2.5 Hz, 1H), |
| | | | | | 7.87 (dd, J = 1.6, 0.8 Hz, 1H), |
| | | | | | 7.85 (d, J = 1.3 Hz, 1H), 7.65 (d, J = 0.9 Hz, |
| | | | | | 1H), 7.65 (d, J = 1.7 Hz, |
| | | | | | 1H), 3.41 (s, 3H), 2.25 (d, J = 1.2 Hz, |
| | | | | | 3H). |
| 104 | 104 | 443.53 | N-(4- | 95.0% | ¹H NMR (400 MHz, |
| | | | methanesulfonylpyridin- | [M + H]⁺ = 444.1 | DMSO) δ 8.94 (d, J = 5.1 Hz, |
| | | | 3-yl)-N- | | 1H), 8.85 (s, 1H), 8.80 (d, J = 1.8 Hz, |
| | | | methyl-8-(1- | | 1H), 8.67 (d, J = 1.8 Hz, |
| | | | methyl-1H-indol- | | 1H), 8.04 (dd, J = 5.1, 0.6 Hz, |
| | | | 6-yl)quinoxalin-6- | | 1H), 7.59-7.54 (m, J = 8.0 Hz, |
| | | | amine | | 2H), 7.38 (d, J = 3.0 Hz, 1H), |
| | | | | | 7.22-7.16 (m, 3H), 6.45 (dd, J = 3.0, |
| | | | | | 0.7 Hz, 1H), 3.78 (s, 3H), |
| | | | | | 3.48 (s, 3H), 3.38 (s, 3H). |
| 110 | 110 | 446.54 | N-(4- | 94.0% | ¹H NMR (400 MHz, |
| | | | methanesulfonylpyridin- | [M + H]⁺ = 447.1 | DMSO) δ 9.01 (s, 1H), 8.83 (d, |
| | | | 3-yl)-8-(3- | | J = 1.8 Hz, 1H), 8.72 (d, J = 1.8 Hz, |
| | | | methyl-1- | | 1H), 8.57 (d, J = 5.1 Hz, |
| | | | benzothiophen-5- | | 1H), 8.48 (s, 1H), 8.07 (d, J = 8.3 Hz, |
| | | | yl)quinoxalin-6- | | 1H), 8.01 (d, J = 1.4 Hz, |
| | | | amine | | 1H), 7.90 (d, J = 2.6 Hz, 1H), |
| | | | | | 7.85 (d, J = 5.1 Hz, 1H), |
| | | | | | 7.66 (dd, J = 8.3, 1.6 Hz, 1H), |
| | | | | | 7.58 (d, J = 2.6 Hz, 1H), 7.47 (d, J = 1.1 Hz, |
| | | | | | 1H), 3.38 (s, 3H), |
| | | | | | 2.44 (d, J = 1.1 Hz, 3H). |
| 113 | 113 | 355.41 | N-(1-methyl-1H- | 93.1% | ¹H NMR (400 MHz, |
| | | | 1,2,3-triazol-5-yl)- | [M + H]⁺ = 356.1 | DMSO) δ 9.02 (s, 1H), 8.79 (d, |
| | | | 8-(1-methyl-1H- | | J = 1.8 Hz, 1H), 8.68 (d, J = 1.9 Hz, |
| | | | indol-6- | | 1H), 7.85 (s, 1H), 7.71 (dt, J = 1.6, |
| | | | yl)quinoxalin-6- | | 0.8 Hz, 1H), 7.65 (d, J = 2.6 Hz, |
| | | | amine | | 1H), 7.64 (dd, J = 8.2, |
| | | | | | 0.7 Hz, 1H), 7.41 (d, J = 3.1 Hz, |
| | | | | | 1H), 7.33 (dd, J = 8.2, 1.4 Hz, |
| | | | | | 1H), 7.20 (d, J = 2.6 Hz, 1H), |
| | | | | | 6.50 (dd, J = 3.0, 0.7 Hz, 1H), |
| | | | | | 3.96 (s, 3H), 3.84 (s, 3H). |
| 115 | 115 | 486.55 | methyl 4- | 99.4% | ¹H NMR (400 MHz, |
| | | | methanesulfonyl- | [M + H]⁺ = 487.1 | DMSO) δ 8.84 (d, J = 1.8 Hz, |
| | | | 3-{[8-(1-methyl- | | 1H), 8.76 (d, J = 1.8 Hz, 1H), |
| | | | 1H-indol-6- | | 8.49 (s, 1H), 8.19 (d, J = 1.4 Hz, |
| | | | yl)quinoxalin-6- | | 1H), 8.07 (d, J = 8.3 Hz, 1H), |
| | | | yl]amino}benzoate | | 7.88 (d, J = 2.6 Hz, 1H), |
| | | | | | 7.80 (dd, J = 8.3, 1.5 Hz, 1H), |
| | | | | | 7.71 (s, 1H), 7.66 (d, J = 2.5 Hz, 1H), |
| | | | | | 7.64 (d, J = 8.2 Hz, 1H), |
| | | | | | 7.41 (d, J = 3.1 Hz, 1H), 7.37 (dd, J = 8.2, |
| | | | | | 1.4 Hz, 1H), 6.49 (dd, J = 3.0, |
| | | | | | 0.8 Hz, 1H), 3.87 (s, 3H), |
| | | | | | 3.84 (s, 3H), 3.35 (s, 3H). |
| 116 | 116 | 471.54 | 4- | 99.0% | ¹NMR (400 MHz, |
| | | | methanesulfonyl- | [M + H]⁺ = 472.1 | DMSO) δ 8.82 (d, J = 1.8 Hz, |
| | | | 3-{[8-(1-methyl- | | 1H), 8.74 (d, J = 1.8 Hz, 1H), |
| | | | 1H-indol-6- | | 8.44 (s, 1H), 8.23 (s, 1H), |
| | | | yl)quinoxalin-6- | | 8.21 (d, J = 1.4 Hz, 1H), 8.00 (d, J = 8.3 Hz, |
| | | | yl]amino}benzamide | | 1H), 7.85 (d, J = 2.6 Hz, |
| | | | | | 1H), 7.75 (dd, J = 8.3, 1.5 Hz, |
| | | | | | 1H), 7.71 (m, 1H), 7.66 (s, 1H), |
| | | | | | 7.63 (d, J = 10.5 Hz, 1H), |
| | | | | | 7.62 (s, 1H), 7.41 (d, J = 3.1 Hz, 1H), |
| | | | | | 7.37 (dd, J = 8.2, 1.4 Hz, 1H), |
| | | | | | 6.49 (dd, J = 3.1, 0.8 Hz, 1H), |
| | | | | | 3.84 (s, 3H), 3.32 (s, 3H). |
| 117 | 117 | 434.49 | 8-(2,1,3- | 100.0% | ¹H NMR (400 MHz, |
| | | | benzothiadiazol-5- | [M + H]⁺ = 435.1 | DMSO) δ 9.04 (s, 1H), 8.89 (d, |
| | | | yl)-N-(4- | | J = 1.8 Hz, 1H), 8.76 (d, J = 1.8 Hz, |
| | | | methanesulfonylpyridin- | | 1H), 8.59 (d, J = 5.0 Hz, |
| | | | 3- | | 1H), 8.53 (s, 1H), 8.36 (dd, J = 1.6, |
| | | | yl)quinoxalin-6- | | 0.8 Hz, 1H), 8.19 (dd, J = 9.1, |
| | | | amine | | 0.7 Hz, 1H), 8.06 (dd, J = 9.1, |
| | | | | | 1.6 Hz, 1H), 8.02 (d, J = 2.6 Hz, |
| | | | | | 1H), 7.86 (dd, J = 5.1, 0.6 Hz, |
| | | | | | 1H), 7.69 (d, J = 2.6 Hz, |
| | | | | | 1H), 3.39 (s, 3H), 2.08 (s, 1H). |
| 118 | 118 | 417.45 | 8-(1H-1,2,3- | 100.0% | ¹H NMR (400 MHz, |
| | | | benzotriazol-5-yl)- | [M + H]⁺ = 418.1 | DMSO) δ 9.02 (s, 1H), 8.85 (d, |
| | | | N-(4- | | J = 1.8 Hz, 1H), 8.74 (d, J = 1.8 Hz, |
| | | | methanesulfonylpyridin- | | 1H), 8.57 (d, J = 5.1 Hz, |
| | | | 3- | | 1H), 8.49 (s, 1H), 8.17 (s, 1H), |
| | | | yl)quinoxalin-6- | | 8.00 (d, J = 8.5 Hz, 1H), |
| | | | amine | | 7.92 (d, J = 2.6 Hz, 1H), 7.85 (d, J = 5.1 Hz, |
| | | | | | 1H), 7.75 (dd, J = 8.6, |
| | | | | | 1.4 Hz, 1H), 7.63 (d, J = 2.6 Hz, |
| | | | | | 1H), 3.38 (s, 3H), 2.54 (s, 1H). |
| 119 | 119 | 486.55 | 4- | 95.2% | ¹H NMR (400 MHz, |
| | | | methanesulfonyl- | [M + H]⁺ = 487.1 | DMSO) δ 10.06 (s, 1H), |
| | | | 3-{[8-(1-methyl- | | 8.83 (d, J = 1.8 Hz, 1H), 8.74 (d, J = 1.8 Hz, |
| | | | 1H-indol-6- | | 1H), 8.43 (s, 1H), |
| | | | yl)quinoxalin-6- | | 8.15 (d, J = 1.3 Hz, 1H), 7.99 (d, J = 8.3 Hz, |
| | | | yl]amino}benzohydrazide | | 1H), 7.85 (d, J = 2.6 Hz, |
| | | | | | 1H), 7.72 (m, 1H), 7.68 (dd, J = 8.3, |
| | | | | | 1.5 Hz, 1H), 7.63 (dd, J = 8.1, |
| | | | | | 0.7 Hz, 1H), 7.63 (d, J = 2.8 Hz, |
| | | | | | 1H), 7.41 (d, J = 3.0 Hz, |
| | | | | | 1H), 7.37 (dd, J = 8.2, 1.4 Hz, |
| | | | | | 1H), 6.49 (dd, J = 3.1, 0.9 Hz, |
| | | | | | 2H), 4.57 (s, 2H), 3.84 (s, 3H), |
| | | | | | 3.32 (s, 3H). |
| 120 | 120 | 418.43 | 8-(2,1,3- | 100.0% | ¹H NMR (400 MHz, |
| | | | benzoxadiazol-5- | [M + H]⁺ = 419.1 | DMSO) δ 9.01 (s, 1H), 8.89 (d, |
| | | | yl)-N-(4- | | J = 1.8 Hz, 1H), 8.76 (d, J = 1.8 Hz, |
| | | | methanesulfonylpyridin- | | 1H), 8.58 (d, J = 5.1 Hz, |
| | | | 3- | | 1H), 8.53 (s, 1H), 8.29 (t, J = 1.1 Hz, |
| | | | yl)quinoxalin-6- | | 1H), 8.13 (dd, J = 9.3, |
| | | | amine | | 0.9 Hz, 1H), 8.01 (d, J = 2.5 Hz, |
| | | | | | 1H), 7.93 (dd, J = 9.3, 1.3 Hz, |
| | | | | | 1H), 7.86 (dd, J = 5.0, 0.5 Hz, |
| | | | | | 1H), 7.70 (d, J = 2.5 Hz, 1H), |
| | | | | | 3.38 (s, 3H), 2.07 (s, 0.25H—HCOOH). |
| 121 | 121 | 505.58 | N-(1- | 96.8% | ¹H NMR (400 MHz, |
| | | | acetylpyrrolidin-3- | [M + H]⁺ = 506.2 | DMSO) δ 9.20 (s, 1H), |
| | | | yl)-3-{[8-(1- | | 8.90 (dd, J = 17.0, 6.7 Hz, 1H), |
| | | | methyl-1H-indol- | | 8.84 (d, J = 6.8 Hz, 1H), 8.76 (d, J = 1.8 Hz, |
| | | | 6-yl)quinoxalin-6- | | 1H), 8.66 (d, J = 1.7 Hz, |
| | | | yl]amino}pyridine- | | 1H), 8.37 (dd, J = 4.9, 4.1 Hz, |
| | | | 4-carboxamide | | 1H), 7.72 (ddt, J = 2.8, 1.3, 0.7 Hz, |
| | | | | | 1H), 7.66 (dd, J = 6.4, 2.6 Hz, |
| | | | | | 1H), 7.62 (d, J = 8.2 Hz, |
| | | | | | 1H), 7.58 (dd, J = 4.7, 3.8 Hz, |
| | | | | | 1H), 7.41 (d, J = 3.2 Hz, 1H), |
| | | | | | 7.40 (dd, J = 8.0, 2.6 Hz, 1H), |
| | | | | | 7.34 (dt, J = 8.1, 1.7 Hz, 1H), |
| | | | | | 6.48 (d, J = 3.1 Hz, 1H), |
| | | | | | 4.34 (dq, J = 23.5, 5.8 Hz, 1H), |
| | | | | | 3.84 (s, 3H), 3.57-3.36 (m, 2H), |
| | | | | | 3.30-3.14 (m, 2H), |
| | | | | | 2.05-1.72 (m, 3H), 1.86 (s, 1.5H), |
| | | | | | 1.76 (s, 1.5H). |
| 122 | 122 | 505.58 | 3-{[8-(1-methyl- | 98.1% | ¹H NMR (400 MHz, |
| | | | 1H-indol-6- | [M + H]⁺ = 506.2 | DMSO) δ 9.17 (s, 1H), 8.86 (d, |
| | | | yl)quinoxalin-6- | | J = 7.3 Hz, 1H), 8.82 (s, 1H), |
| | | | yl]amino}-N-(1- | | 8.77 (d, J = 1.8 Hz, 1H), |
| | | | methyl-6- | | 8.66 (d, J = 1.8 Hz, 1H), 8.37 (d, J = 4.9 Hz, |
| | | | oxopiperidin-3- | | 1H), 7.72-7.69 (m, |
| | | | yl)pyridine-4- | | 1H), 7.64 (d, J = 2.6 Hz, 1H), |
| | | | carboxamide | | 7.63 (dd, J = 8.2, 0.6 Hz, 1H), |
| | | | | | 7.57 (dd, J = 5.0, 0.6 Hz, 1H), |
| | | | | | 7.40 (d, J = 3.1 Hz, 1H), |
| | | | | | 7.38 (d, J = 2.6 Hz, 1H), 7.33 (dd, J = 8.2, |
| | | | | | 1.4 Hz, 1H), 6.48 (dd, J = 3.0, |
| | | | | | 0.6 Hz, 1H), |
| | | | | | 4.18-4.10 (m, 1H), 3.83 (s, 3H), 3.24 (dd, |
| | | | | | J = 11.9, 5.0 Hz, 1H), 2.99 (dd, |
| | | | | | J = 12.1, 7.6 Hz, 1H), 2.62 (s, |
| | | | | | 3H), 2.31-2.13 (m, 2H), |
| | | | | | 1.86-1.68 (m, 2H). |
| 123 | 123 | 491.60 | 3-{[8-(1-methyl- | 97.2% | ¹H NMR (400 MHz, |
| | | | 1H-indol-6- | [M + H]⁺ = 492.2 | DMSO) δ 9.28 (s, 1H), 8.86 (s, |
| | | | yl)quinoxalin-6- | | 1H), 8.77 (d, J = 1.8 Hz, 1H), |
| | | | yl]amino}-N-(1- | | 8.66 (d, J = 1.9 Hz, 1H), |
| | | | methylpiperidin-4- | | 8.60 (d, J = 7.6 Hz, 1H), 8.34 (d, J = 4.9 Hz, |
| | | | yl)pyridine-4- | | 1H), 7.71-7.68 (m, |
| | | | carboxamide | | 1H), 7.66 (d, J = 2.6 Hz, 1H), |
| | | | | | 7.62 (d, J = 8.2 Hz, 1H), |
| | | | | | 7.57 (d, J = 5.0 Hz, 1H), 7.43 (d, J = 2.6 Hz, |
| | | | | | 1H), 7.40 (d, J = 3.1 Hz, |
| | | | | | 1H), 7.32 (dd, J = 8.2, 1.4 Hz, |
| | | | | | 1H), 6.48 (dd, J = 3.0, 0.7 Hz, |
| | | | | | 1H), 3.83 (s, 3H), |
| | | | | | 3.69-3.58 (m, 1H), 2.72-2.61 (m, 2H), |
| | | | | | 2.11 (s, 3H), 1.97-1.84 (m, |
| | | | | | 2H), 1.69-1.60 (m, 2H), |
| | | | | | 1.51-1.39 (m, 2H). |
| 124 | 124 | 491.60 | 3-{[8-(1-methyl- | 95.7% | ¹H NMR (400 MHz, |
| | | | 1H-indol-6- | [M + H]⁺ = 492.2 | DMSO) δ 9.19 (s, 1H), 8.80 (s, |
| | | | yl)quinoxalin-6- | | 1H), 8.76 (d, J = 1.8 Hz, 1H), |
| | | | yl]amino}-N-(1- | | 8.65 (d, J = 1.8 Hz, 1H), |
| | | | methylpiperidin-3- | | 8.48 (d, J = 8.0 Hz, 1H), 8.37 (d, J = 5.0 Hz, |
| | | | yl)pyridine-4- | | 1H), 7.71-7.68 (m, 1H), |
| | | | carboxamide | | 7.63 (d, J = 8.1 Hz, 1H), |
| | | | | | 7.63 (d, J = 2.9 Hz, 1H), 7.56 (d, J = 5.0 Hz, |
| | | | | | 1H), 7.40 (d, J = 3.1 Hz, |
| | | | | | 1H), 7.35 (d, J = 2.8 Hz, 1H), |
| | | | | | 7.34 (dd, J = 8.2, 1.5 Hz, 1H), |
| | | | | | 6.48 (dd, J = 3.0, 0.6 Hz, 1H), |
| | | | | | 3.83 (s, 3H), 3.81-3.73 (m, |
| | | | | | 1H), 2.49-2.39 (m, 2H), |
| | | | | | 1.94 (s, 3H), 1.80-1.69 (m, |
| | | | | | 1H), 1.66-1.49 (m, 3H), |
| | | | | | 1.44-1.30 (m, 1H), 1.22-1.09 (m, |
| | | | | | 1H). |
| 125 | 125 | 486.54 | 3-{methyl[8-(1- | 95.2% | ¹H NMR (400 MHz, |
| | | | methyl-1H-indol- | [M + H]⁺ = 487.2 | DMSO) δ 10.94 (s, 1H), 8.84 (s, |
| | | | 6-yl)quinoxalin-6- | | 1H), 8.80 (s, 1H), 8.72 (d, J = 1.8 Hz, |
| | | | yl]amino}-N- | | 1H), 8.69 (d, J = 5.0 Hz, |
| | | | (pyrimidin-5- | | 1H), 8.68 (s, 1H), 8.56 (d, J = 1.8 Hz, |
| | | | yl)pyridine-4- | | 1H), 7.67 (d, J = 4.9 Hz, |
| | | | carboxamide | | 1H), 7.54 (d, J = 8.2 Hz, 1H), |
| | | | | | 7.44-7.41 (m, 1H), 7.36 (d, J = 3.1 Hz, |
| | | | | | 1H), 7.20 (d, J = 2.8 Hz, |
| | | | | | 1H), 7.18 (d, J = 2.8 Hz, |
| | | | | | 1H), 7.06 (dd, J = 8.2, 1.4 Hz, |
| | | | | | 1H), 6.44 (dd, J = 3.0, 0.6 Hz, |
| | | | | | 1H), 3.77 (s, 3H), 3.53 (s, 3H). |
| 126 | 126 | 476.58 | N-cyclohexyl-3- | 98.3% | ¹H NMR (400 MHz, DMSO-d₆) |
| | | | {[8-(1-methyl-1H- | [M + H]⁺ = 477.2 | δ 9.30 (s, 1H), 8.87 (s, 1H), |
| | | | indol-6- | | 8.78 (d, J = 1.8 Hz, 1H), |
| | | | yl)quinoxalin-6- | | 8.67 (d, J = 1.8 Hz, 1H), 8.55 (d, J = 7.7 Hz, |
| | | | yl]amino}pyridine- | | 1H), 8.35 (d, J = 5.0 Hz, |
| | | | 4-carboxamide | | 1H), 7.71 (dt, J = 1.5, 0.8 Hz, |
| | | | | | 1H), 7.67 (d, J = 2.6 Hz, 1H), |
| | | | | | 7.63 (d, J = 8.2 Hz, 1H), |
| | | | | | 7.57 (d, J = 5.0 Hz, 1H), 7.45 (d, J = 2.6 Hz, |
| | | | | | 1H), 7.40 (d, J = 3.0 Hz, |
| | | | | | 1H), 7.33 (dd, J = 8.2, 1.5 Hz, |
| | | | | | 1H), 6.49 (dd, J = 3.1, 0.8 Hz, |
| | | | | | 1H), 3.84 (s, 3H), |
| | | | | | 3.73-3.61 (m, 1H), 1.75-1.49 (m, 5H), |
| | | | | | 1.30-1.00 (m, 5H). |
| 127 | 127 | 491.56 | 3-{[8-(1-methyl- | 98.3% | ¹H NMR (400 MHz, DMSO-d₆) |
| | | | 1H-indol-6- | [M + H]⁺ = 492.2 | δ 9.25 (s, 1H), 8.87 (s, 1H), |
| | | | yl)quinoxalin-6- | | 8.80 (d, J = 7.4 Hz, 1H), |
| | | | yl]amino}-N-(2- | | 8.78 (d, J = 1.9 Hz, 1H), 8.67 (dd, J = 1.9, |
| | | | oxopiperidin-4- | | 0.6 Hz, 1H), 8.36 (d, J = 5.0 Hz, |
| | | | yl)pyridine-4- | | 1H), 7.73-7.71 (m, 1H), |
| | | | carboxamide | | 7.67 (d, J = 2.6 Hz, 1H), |
| | | | | | 7.63 (d, J = 8.2 Hz, 1H), 7.59 (d, J = 5.0 Hz, |
| | | | | | 1H), 7.52 (s, 1H), |
| | | | | | 7.44 (d, J = 2.6 Hz, 1H), 7.40 (d, J = 3.1 Hz, |
| | | | | | 1H), 7.34 (dd, J = 8.2, |
| | | | | | 1.0 Hz, 1H), 6.49 (d, J = 3.0 Hz, |
| | | | | | 1H), 4.16-4.05 (m, 1H), |
| | | | | | 3.84 (s, 3H), 3.18-3.01 (m, 2H), |
| | | | | | 2.35 (dd, J = 17.3, 5.8 Hz, 1H), |
| | | | | | 2.13 (dd, J = 17.3, 9.0 Hz, 1H), |
| | | | | | 1.83 (dd, J = 13.0, 4.5 Hz, 1H), |
| | | | | | 1.58 (dtd, J = 14.5, 9.6, 5.3 Hz, |
| | | | | | 1H). |
| 128 | 128 | 433.49 | 2-{7-[(4- | 94.1% | ¹H NMR (400 MHz, |
| | | | methanesulfonylpyridin- | [M + H]⁺ = 434.2 | DMSO) δ 8.94 (s, 1H), 8.77 (d, |
| | | | 3- | | J = 1.8 Hz, 1H), 8.60 (d, J = 1.9 Hz, |
| | | | yl)amino]quinoxalin- | | 1H), 8.54 (d, J = 5.1 Hz, |
| | | | 5-yl}-4- | | 1H), 8.43 (s, 1H), 7.83 (d, J = 5.0 Hz, |
| | | | methylbenzamide | | 1H), 7.63 (d, J = 2.5 Hz, |
| | | | | | 1H), 7.60 (d, J = 2.5 Hz, 1H), |
| | | | | | 7.55 (d, J = 7.8 Hz, 1H), |
| | | | | | 7.46 (m, 1H), 7.31-7.28 (m, 1H), |
| | | | | | 7.23 (m, 1H), 6.90 (s, 1H), |
| | | | | | 3.37 (s, 3H), 2.38 (s, 3H). |
| 129 | 129 | 420.49 | 8-(3- | 99.5% | ¹H NMR (400 MHz, |
| | | | ethoxyphenyl)-N- | [M + H]⁺ = 421.1 | DMSO) δ 8.99 (s, 1H), 8.83 (d, |
| | | | (4- | | J = 1.9 Hz, 1H), 8.73 (d, J = 1.9 Hz, |
| | | | methanesulfonylpyridin- | | 1H), 8.57 (d, J = 5.1 Hz, |
| | | | 3- | | 1H), 8.45 (s, 1H), 7.85 (dd, J = 5.1, |
| | | | yl)quinoxalin-6- | | 0.5 Hz, 1H), 7.82 (d, J = 2.6 Hz, |
| | | | amine | | 1H), 7.59 (d, J = 2.6 Hz, |
| | | | | | 1H), 7.40 (t, J = 8.1 Hz, 1H), |
| | | | | | 7.23-7.19 (m, 2H), 7.01 (ddd, |
| | | | | | J = 8.3, 2.5, 1.0 Hz, 1H), |
| | | | | | 4.08 (q, J = 7.0 Hz, 2H), 3.37 (s, 3H), |
| | | | | | 1.36 (t, J = 7.0 Hz, 3H). |
| 130 | 130 | 434.51 | N-(4- | 99.2% | ¹H NMR (400 MHz, |
| | | | methanesulfonylpyridin- | [M + H]⁺ = 435.1 | DMSO) δ 9.00 (s, 1H), 8.83 (d, |
| | | | 3-yl)-8-[3- | | J = 1.8 Hz, 1H), 8.73 (d, J = 1.8 Hz, |
| | | | (propan-2- | | 1H), 8.57 (d, J = 5.1 Hz, |
| | | | yloxy)phenyl]quinoxalin- | | 1H), 8.45 (s, 1H), 7.85 (dd, J = 5.1, |
| | | | 6-amine | | 0.5 Hz, 1H), 7.81 (d, J = 2.6 Hz, |
| | | | | | 1H), 7.58 (d, J = 2.6 Hz, |
| | | | | | 1H), 7.38 (t, J = 8.1 Hz, 1H), |
| | | | | | 7.19 (ddd, J = 5.2, 2.6, 1.2 Hz, |
| | | | | | 2H), 7.00 (ddd, J = 8.3, 2.5, 0.9 Hz, |
| | | | | | 1H), 4.67 (hept, J = 6.0 Hz, |
| | | | | | 1H), 3.37 (s, 3H), 1.31 (d, J = 6.0 Hz, |
| | | | | | 6H). |
| 131 | 131 | 391.45 | 8-(4- | 99.6% | ¹H NMR (400 MHz, |
| | | | aminophenyl)-N- | [M + H]⁺ = 392.1 | DMSO) δ 8.97 (s, 1H), 8.78 (d, |
| | | | (4- | | J = 1.8 Hz, 1H), 8.71 (d, J = 1.8 Hz, |
| | | | methanesulfonylpyridin- | | 1H), 8.55 (d, J = 5.1 Hz, |
| | | | 3- | | 1H), 8.38 (s, 1H), 7.84 (d, J = 5.1 Hz, |
| | | | yl)quinoxalin-6- | | 1H), 7.70 (d, J = 2.6 Hz, |
| | | | amine | | 1H), 7.48 (d, J = 2.6 Hz, 1H), |
| | | | | | 7.44-7.38 (m, 2H), |
| | | | | | 6.70-6.64 (m, 2H), 5.30 (s, 2H), |
| | | | | | 3.37 (s, 3H). |
| 132 | 132 | 391.45 | 8-(3- | 98.8% | ¹H NMR (400 MHz, |
| | | | aminophenyl)-N- | [M + H]⁺ = 392.1 | DMSO) δ 8.97 (s, 1H), 8.80 (d, |
| | | | (4- | | J = 1.8 Hz, 1H), 8.72 (d, J = 1.8 Hz, |
| | | | methanesulfonylpyridin- | | 1H), 8.57 (d, J = 5.1 Hz, |
| | | | 3- | | 1H), 8.45 (s, 1H), 7.85 (d, J = 5.1 Hz, |
| | | | yl)quinoxalin-6- | | 1H), 7.71 (d, J = 2.6 Hz, |
| | | | amine | | 1H), 7.53 (d, J = 2.6 Hz, 1H), |
| | | | | | 7.12 (t, J = 7.8 Hz, 1H), 6.84 (t, |
| | | | | | J = 1.9 Hz, 1H), 6.75 (dt, J = 7.8, |
| | | | | | 1.2 Hz, 1H), 6.63 (ddd, J = 7.9, |
| | | | | | 2.2, 0.9 Hz, 1H), 5.14 (s, |
| | | | | | 2H), 3.37 (s, 3H). |
| 135 | 135 | 493.57 | 3-{[8-(1-methyl- | 96.0% | ¹H NMR (400 MHz, |
| | | | 1H-indol-6- | [M + H]⁺ = 494.2 | DMSO) δ 9.26 (s, 1H), 8.87 (s, |
| | | | yl)quinoxalin-6- | | 1H), 8.78 (d, J = 1.8 Hz, 1H), |
| | | | yl]amino}-N- | | 8.73 (t, J = 5.9 Hz, 1H), 8.67 (d, |
| | | | [(morpholin-3- | | J = 1.8 Hz, 1H), 8.36 (d, J = 5.0 Hz, |
| | | | yl)methyl]pyridine- | | 1H), 7.71 (s, 1H), 7.67 (d, J = 2.6 Hz, |
| | | | 4-carboxamide | | 1H), 7.63 (d, J = 8.3 Hz, |
| | | | | | 1H), 7.61 (d, J = 5.1 Hz, |
| | | | | | 1H), 7.42 (d, J = 2.6 Hz, 1H), |
| | | | | | 7.41 (d, J = 3.1 Hz, 1H), |
| | | | | | 7.34 (dd, J = 8.2, 1.4 Hz, 1H), |
| | | | | | 6.49 (dd, J = 3.1, 0.8 Hz, 1H), |
| | | | | | 3.84 (s, 3H), 3.61 (dd, J = 10.9, 2.9 Hz, |
| | | | | | 1H), 3.57-3.49 (m, J = 13.5 Hz, |
| | | | | | 2H), 3.28-3.20 (m, |
| | | | | | 1H), 3.12 (t, J = 6.0 Hz, 2H), |
| | | | | | 3.02 (t, 1H), 2.77-2.70 (m, J = 6.2 Hz, |
| | | | | | 1H), 2.66-2.60 (m, J = 12.1 Hz, |
| | | | | | 1H), 2.58-2.53 (m, |
| | | | | | 1H). |
| 136 | 136 | 535.61 | N-[(4- | 96.3% | no NMR taken |
| | | | acetylmorpholin- | [M + H]⁺ = 536.2 | |
| | | | 3-yl)methyl]-3-{[8- | | |
| | | | (1-methyl-1H- | | |
| | | | indol-6- | | |
| | | | yl)quinoxalin-6- | | |
| | | | yl]amino}pyridine- | | |
| | | | 4-carboxamide | | |
| 137 | 137 | 507.60 | 3-{[8-(1-methyl- | 98.2% | ¹H NMR (400 MHz, |
| | | | 1H-indol-6- | [M + H]⁺ = 508.2 | DMSO) δ 9.28 (s, 1H), 8.88 (s, |
| | | | yl)quinoxalin-6- | | 1H), 8.84 (t, J = 5.9 Hz, 1H), |
| | | | yl]amino}-N-[(4- | | 8.78 (d, J = 1.8 Hz, 1H), |
| | | | methylmorpholin- | | 8.67 (d, J = 1.8 Hz, 1H), 8.37 (d, J = 4.8 Hz, |
| | | | 2- | | 1H), 7.71 (s, 1H), |
| | | | yl)methyl]pyridine- | | 7.68 (d, J = 2.6 Hz, 1H), 7.63 (d, J = 8.2 Hz, |
| | | | 4-carboxamide | | 1H), 7.60 (d, J = 5.0 Hz, |
| | | | | | 1H), 7.43 (d, J = 2.6 Hz, 1H), |
| | | | | | 7.40 (d, J = 3.1 Hz, 1H), |
| | | | | | 7.34 (dd, J = 8.2, 1.4 Hz, 1H), |
| | | | | | 6.49 (dd, J = 3.0, 0.8 Hz, 1H), |
| | | | | | 3.84 (s, 3H), 3.68 (ddd, J = 11.0, 2.8, |
| | | | | | 1.7 Hz, 2H), 3.53-3.44 (m, |
| | | | | | 1H), 3.41-3.35 (m, 1H), |
| | | | | | 3.30-3.20 (m, 2H), 2.59 (d, J = 11.2 Hz, |
| | | | | | 1H), 2.47 (d, J = 12.0 Hz, |
| | | | | | 1H), 2.02 (s, 3H), 1.85 (td, J = 11.2, |
| | | | | | 3.0 Hz, 1H), 1.63 (t, J = 10.5 Hz, |
| | | | | | 1H). |
| 140 | 140 | 505.58 | N-[(1- | 99.6% | ¹H NMR (400 MHz, |
| | | | acetylazetidin-3- | [M + H]⁺ = 506.2 | DMSO) δ 9.26 (s, 1H), 8.90 (t, J = 5.7 Hz, |
| | | | yl)methyl]-3-{[8- | | 1H), 8.87 (s, 1H), |
| | | | (1-methyl-1H- | | 8.78 (d, J = 1.8 Hz, 1H), 8.67 (d, J = 1.8 Hz, |
| | | | indol-6- | | 1H), 8.37 (d, J = 5.0 Hz, |
| | | | yl)quinoxalin-6- | | 1H), 7.71 (s, 1H), 7.68 (d, J = 2.6 Hz, |
| | | | yl]amino}pyridine- | | 1H), 7.63 (d, J = 8.2 Hz, |
| | | | 4-carboxamide | | 1H), 7.56 (d, J = 4.9 Hz, 1H), |
| | | | | | 7.42 (d, J = 2.6 Hz, 1H), |
| | | | | | 7.41 (d, J = 3.1 Hz, 1H), 7.34 (dd, J = 8.2, |
| | | | | | 1.4 Hz, 1H), 6.49 (d, J = 3.1 Hz, |
| | | | | | 1H), 3.95 (t, J = 8.4 Hz, 1H), |
| | | | | | 3.84 (s, 3H), 3.76-3.69 (m, |
| | | | | | 2H), 3.54-3.47 (m, 2H), |
| | | | | | 3.43-3.38 (m, 1H), 1.61 (s, 3H). |
| 141 | 141 | 535.61 | N-[(4- | 100.0% | ¹H NMR (400 MHz, DMSO-d₆) |
| | | | acetylmorpholin- | [M + H]⁺ = 536.2 | δ 9.29 (d, J = 42.4 Hz, 1H), |
| | | | 2-yl)methyl]-3-{[8- | | 8.98-8.84 (m, 2H), 8.78 (dd, J = 7.9, |
| | | | (1-methyl-1H- | | 1.9 Hz, 1H), 8.67 (dd, J = 6.6, |
| | | | indol-6- | | 1.9 Hz, 1H), 8.36 (dd, J = 9.9, |
| | | | yl)quinoxalin-6- | | 5.0 Hz, 1H), 7.71 (d, J = 8.6 Hz, |
| | | | yl]amino}pyridine- | | 1H), 7.69 (dd, J = 8.3, 2.5 Hz, |
| | | | 4-carboxamide | | 1H), 7.63 (dd, J = 8.2, 0.6 Hz, |
| | | | | | 1H), 7.60 (t, J = 4.4, 4.0 Hz, |
| | | | | | 1H), 7.46 (dd, J = 24.8, 2.6 Hz, |
| | | | | | 1H), 7.41 (d, J = 3.1 Hz, 1H), |
| | | | | | 7.33 (td, J = 8.2, 1.3 Hz, 2H), |
| | | | | | 6.49 (dd, J = 3.1, 0.8 Hz, 1H), |
| | | | | | 4.12 (dd, J = 93.2, 13.2 Hz, |
| | | | | | 1H), 3.84 (s, 3H), 3.75 (d, J = 11.7 Hz, |
| | | | | | 1H), 3.62 (dd, J = 39.8, |
| | | | | | 13.2 Hz, 1H), |
| | | | | | 3.28-2.95 (m, 5H), 1.90 (d, J = 34.8 Hz, |
| | | | | | 3H). |
| 142 | 142 | 491.60 | 3-{[8-(1-methyl- | 91.1% | ¹NMR (400 MHz, |
| | | | 1H-indol-6- | [M + H]⁺ = 492.3 | DMSO) δ 9.26 (s, 1H), 8.86 (s, |
| | | | yl)quinoxalin-6- | | 1H), 8.79 (t, J = 5.3 Hz, 2H), |
| | | | yl]amino}-N-[(1- | | 8.77 (d, J = 1.9 Hz, 1H), |
| | | | methylpyrrolidin- | | 8.66 (d, J = 1.8 Hz, 1H), 8.36 (d, J = 5.0 Hz, |
| | | | 3- | | 1H), 7.71-7.68 (m, 1H), |
| | | | yl)methyl]pyridine- | | 7.66 (d, J = 2.6 Hz, 1H), |
| | | | 4-carboxamide | | 7.62 (d, J = 8.2 Hz, 1H), 7.56 (d, J = 5.0 Hz, |
| | | | | | 1H), 7.41 (d, J = 2.6 Hz, |
| | | | | | 1H), 7.41 (d, J = 3.3 Hz, 2H), |
| | | | | | 7.32 (dd, J = 8.2, 1.4 Hz, 1H), |
| | | | | | 6.48 (dd, J = 3.0, 0.5 Hz, 1H), |
| | | | | | 3.83 (s, 3H), 3.14 (t, J = 6.0 Hz, |
| | | | | | 2H), 2.39 (td, J = 8.4, 5.1 Hz, |
| | | | | | 1H), 2.29-2.17 (m, 3H), |
| | | | | | 2.16-2.11 (m, 1H), 2.08 (s, 3H), |
| | | | | | 1.80-1.69 (m, 1H), |
| | | | | | 1.39-1.28 (m, 1H). |
| 143 | 143 | 488.56 | N-[(1-methyl-1H- | 95.1% | ¹H NMR (400 MHz, |
| | | | imidazol-5- | [M + H]⁺ = 489.2 | DMSO) δ 9.28 (s, 1H), 9.13 (t, J = 5.4 Hz, |
| | | | yl)methyl]-3-{[8- | | 1H), 8.88 (s, 1H), |
| | | | (1-methyl-1H- | | 8.77 (d, J = 1.8 Hz, 1H), 8.67 (d, J = 1.9 Hz, |
| | | | indol-6- | | 1H), 8.33 (d, J = 5.0 Hz, |
| | | | yl)quinoxalin-6- | | 1H), 7.72 (dt, J = 1.5, 0.8 Hz, |
| | | | yl]amino}pyridine- | | 1H), 7.68 (d, J = 2.6 Hz, 1H), |
| | | | 4-carboxamide | | 7.63 (dd, J = 8.2, 0.7 Hz, 1H), |
| | | | | | 7.59 (dd, J = 5.0, 0.6 Hz, 1H), |
| | | | | | 7.46 (d, J = 0.6 Hz, 1H), |
| | | | | | 7.46 (d, J = 2.6 Hz, 2H), 7.40 (d, J = 3.1 Hz, |
| | | | | | 1H), 7.34 (dd, J = 8.2, |
| | | | | | 1.4 Hz, 1H), 6.80 (d, J = 1.1 Hz, |
| | | | | | 1H), 6.49 (dd, J = 3.1, 0.6 Hz, |
| | | | | | 1H), 4.41 (d, J = 5.3 Hz, 2H), |
| | | | | | 3.84 (s, 3H), 3.52 (s, 3H). |
| 144 | 144 | 486.54 | 3-{[8-(1-methyl- | 97.0% | ¹H NMR (400 MHz, |
| | | | 1H-indol-6- | [M + H]⁺ = 487.2 | DMSO) δ 9.50 (t, J = 5.9 Hz, |
| | | | yl)quinoxalin-6- | | 1H), 9.35 (s, 1H), 9.06 (dd, J = 4.9, |
| | | | yl]amino}-N- | | 1.6 Hz, 1H), 8.91 (s, 1H), |
| | | | [(pyridazin-3- | | 8.77 (d, J = 1.8 Hz, 1H), |
| | | | yl)methyl]pyridine- | | 8.68 (d, J = 1.8 Hz, 1H), 8.37 (d, J = 5.0 Hz, |
| | | | 4-carboxamide | | 1H), 7.71 (d, J = 2.6 Hz, |
| | | | | | 1H), 7.67 (d + m, J = 5.2 Hz, 2H), |
| | | | | | 7.62 (d, J = 8.2 Hz, 1H), |
| | | | | | 7.54 (dd, J = 8.5, 1.6 Hz, 1H), |
| | | | | | 7.47 (d, J = 2.6 Hz, 1H), 7.43 (dd, J = 8.5, |
| | | | | | 4.9 Hz, 1H), 7.40 (d, J = 3.0 Hz, |
| | | | | | 1H), 7.32 (dd, J = 8.2, 1.4 Hz, |
| | | | | | 1H), 6.48 (dd, J = 3.0, 0.5 Hz, |
| | | | | | 1H), 4.73 (d, J = 5.9 Hz, |
| | | | | | 2H), 3.83 (s, 3H). |
| 145 | 145 | 376.42 | 4-{[8-(1-methyl- | 92.8% | ¹H NMR (400 MHz, |
| | | | 1H-indol-6- | [M + H]⁺ = 377.1 | DMSO) δ 9.78 (s, 1H), 8.92 (d, |
| | | | yl)quinoxalin-6- | | J = 1.8 Hz, 1H), 8.86 (d, J = 1.8 Hz, |
| | | | yl]amino}pyridine- | | 1H), 8.76 (s, 1H), 8.46 (d, J = 6.0 Hz, |
| | | | 3-carbonitrile | | 1H), 7.91 (d, J = 2.5 Hz, |
| | | | | | 1H), 7.87 (d, J = 2.5 Hz, |
| | | | | | 1H), 7.75 (s, 1H), 7.65 (d, J = 8.2 Hz, |
| | | | | | 1H), 7.41 (d, J = 9.5 Hz, |
| | | | | | 1H), 7.41 (s, 1H),, 7.38 (dd, J = 8.2, |
| | | | | | 1.3 Hz, 1H), 6.50 (dd, J = 3.1, |
| | | | | | 0.8 Hz, 1H), 3.84 (s, 3H). |
| 146 | 146 | 281.70 | 4-[(8- | 97.4% | ¹H NMR (400 MHz, |
| | | | chloroquinoxalin- | [M + H]⁺ = 281.9 | DMSO) δ 9.79 (s, 1H), 8.99 (d, |
| | | | 6- | | J = 1.8 Hz, 1H), 8.95 (d, J = 1.8 Hz, |
| | | | yl)amino]pyridine- | | 1H), 8.78 (s, 1H), 8.48 (d, J = 6.0 Hz, |
| | | | 3-carbonitrile | | 1H), 8.06 (d, J = 2.3 Hz, |
| | | | | | 1H), 7.85 (d, J = 2.3 Hz, |
| | | | | | 1H), 7.36 (d, J = 6.0 Hz, 1H). |
| 147 | 147 | 519.61 | N-(1- | 100.0% | ¹HNMR (400 MHz, |
| | | | acetylpiperidin-4- | [M + H]⁺ = 520.2 | DMSO) δ 9.25 (s, 1H), 8.85 (s, |
| | | | yl)-3-{[8-(1- | | 1H), 8.78 (d, J = 1.8 Hz, 1H), |
| | | | methyl-1H-indol- | | 8.67 (d, J = 1.9 Hz, 1H), |
| | | | 6-yl)quinoxalin-6- | | 8.64 (d, J = 7.6 Hz, 1H), 8.36 (d, J = 5.0 Hz, |
| | | | yl]amino}pyridine- | | 1H), 7.70 (dt, J = 1.6, |
| | | | 4-carboxamide | | 0.8 Hz, 1H), 7.66 (d, J = 2.6 Hz, |
| | | | | | 1H), 7.63 (dd, J = 8.2, 0.7 Hz, |
| | | | | | 1H), 7.57 (dd, J = 5.0, 0.6 Hz, |
| | | | | | 1H), 7.42 (d, J = 2.6 Hz, 1H), |
| | | | | | 7.41 (d, J = 3.1 Hz, 1H), |
| | | | | | 7.33 (dd, J = 8.2, 1.5 Hz, 1H), |
| | | | | | 6.49 (dd, J = 3.1, 0.8 Hz, 1H), |
| | | | | | 4.15 (m, 1H), 3.91 (m, 1H), 3.84 (s, |
| | | | | | 3H), 3.68 (m, 1H), 3.05 (ddd, J = 14.2, |
| | | | | | 11.8, 2.9 Hz, 1H), |
| | | | | | 2.66 (m, 1H), 1.95 (s, 3H), 1.68 (dd, |
| | | | | | J = 29.4, 10.0 Hz, 2H), |
| | | | | | 1.38-1.17 (m, 2H). |
| 148 | 148 | 519.61 | N-(1- | 99.9% | ¹H NMR (400 MHz, DMSO-d₆) |
| | | | acetylpiperidin-3- | [M + H]⁺ = 520.3 | δ 9.14 (d, J = 10.8 Hz, 1H), |
| | | | yl)-3-{[8-(1- | | 8.85 (d, J = 13.3 Hz, 1H), 8.77 (d, J = 1.8 Hz, |
| | | | methyl-1H-indol- | | 1H), 8.67 (dd, J = 38.1, |
| | | | 6-yl)quinoxalin-6- | | 7.5 Hz, 1H), 8.66 (d, J = 1.8 Hz, |
| | | | yl]amino}pyridine- | | 1H), 8.37 (dd, J = 8.3, 4.9 Hz, |
| | | | 4-carboxamide | | 1H), 7.71 (d, J = 6.2 Hz, 1H), |
| | | | | | 7.67 (dd, J = 14.5, 2.6 Hz, 1H), |
| | | | | | 7.63 (dd, J = 8.3, 0.7 Hz, 1H), |
| | | | | | 7.56 (d, J = 5.0 Hz, 1H), |
| | | | | | 7.42 (dd, J = 13.8, 2.5 Hz, 1H), |
| | | | | | 7.40 (t, J = 2.9, 1.3 Hz, 1H), |
| | | | | | 7.33 (ddd, J = 8.2, 2.7, 1.4 Hz, 1H), |
| | | | | | 6.49 (d, J = 3.1 Hz, 1H), |
| | | | | | 4.10 (dd, J = 12.6, 3.9 Hz, 1H), |
| | | | | | 3.84 (s, 3H), 3.87-3.65 (m, 2H), |
| | | | | | 3.62-3.49 (m, 1H), |
| | | | | | 3.07-2.98 (m, 1H), 2.96-2.87 (m, |
| | | | | | 1H), 2.83-2.68 (m, 2H), |
| | | | | | 1.98 (s, 1H), 1.76 (s, 1H), |
| | | | | | 1.72-1.35 (m, 4H). |
| 149 | 149 | 395.43 | 5-{[8-(1-methyl- | 99.8% | ¹H NMR (400 MHz, |
| | | | 1H-indol-6- | [M + H]⁺ = 396.1 | DMSO) δ 10.46 (s, 1H), 9.29 (s, |
| | | | yl)quinoxalin-6- | | 1H), 8.88 (d, J = 1.8 Hz, 1H), |
| | | | yl]amino}pyrimidine- | | 8.80 (s, 1H), 8.79 (d, J = 1.8 Hz, |
| | | | 4-carboxamide | | 1H), 8.57 (s, 1H), 8.11 (s, 1H), |
| | | | | | 7.86 (d, J = 2.6 Hz, 1H), |
| | | | | | 7.83 (d, J = 2.6 Hz, 1H), 7.76 (s, 1H), |
| | | | | | 7.64 (d, J = 8.2 Hz, 1H), |
| | | | | | 7.41 (d, J = 3.0 Hz, 1H), 7.38 (dd, J = 8.2, |
| | | | | | 1.4 Hz, 1H), 6.49 (dd, J = 3.1, |
| | | | | | 0.8 Hz, 1H), 3.85 (s, 3H). |
| 150 | 150 | 393.47 | 3-{[8-(3-methyl-1- | 97.6% | ¹H NMR (400 MHz, |
| | | | benzothiophen-5- | [M + H]⁺ = 394.1 | DMSO) δ 9.48 (s, 1H), 8.94 (d, |
| | | | yl)quinoxalin-6- | | J = 0.4 Hz, 1H), 8.84 (d, J = 1.8 Hz, |
| | | | yl]amino}pyridine- | | 1H), 8.73 (d, J = 1.8 Hz, |
| | | | 4-carbonitrile | | 1H), 8.42 (d, J = 5.0 Hz, 1H), |
| | | | | | 8.08 (dd, J = 8.3, 0.6 Hz, 1H), |
| | | | | | 8.02 (dd, J = 1.8, 0.6 Hz, 1H), |
| | | | | | 7.84 (dd, J = 5.0, 0.5 Hz, 1H), |
| | | | | | 7.79 (d, J = 2.6 Hz, 1H), |
| | | | | | 7.67 (dd, J = 8.3, 1.6 Hz, 1H), |
| | | | | | 7.54 (d, J = 2.6 Hz, 1H), 7.47 (d, J = 1.1 Hz, |
| | | | | | 1H), 2.44 (d, J = 1.0 Hz, |
| | | | | | 3H). |
| 151 | 151 | 494.62 | 3-{[8-(3-methyl-1- | 96.2% | ¹H NMR (400 MHz, DMSO) δ |
| | | | benzothiophen-5- | [M + H]⁺ = 495.2 | 9.27 (s, 1H), 8.84 (s, 1H), |
| | | | yl)quinoxalin-6- | | 8.80 (d, J = 7.2 Hz, 1H), 8.80 (d, J = 1.9 Hz, |
| | | | yl]amino}-N-(1- | | 1H), 8.66 (d, J = 1.8 Hz, |
| | | | methylpyrrolidin- | | 1H), 8.35 (d, J = 4.9 Hz, 1H), |
| | | | 3-yl)pyridine-4- | | 8.07 (dd, J = 8.3, 0.6 Hz, 1H), |
| | | | carboxamide | | 8.00 (d, J = 1.2 Hz, 1H), |
| | | | | | 7.70 (d, J = 2.6 Hz, 1H), 7.66 (dd, J = 8.3, |
| | | | | | 1.5 Hz, 1H), 7.58 (d, J = 5.0 Hz, |
| | | | | | 1H), 7.46 (d, J = 1.1 Hz, |
| | | | | | 1H), 7.44 (d, J = 2.6 Hz, 1H), |
| | | | | | 4.27 (dtt, J = 9.4, 7.1, 4.8 Hz, |
| | | | | | 1H), 2.58 (dd, J = 9.6, 7.2 Hz, |
| | | | | | 1H), 2.49-2.45 (m, 1H), |
| | | | | | 2.45 (d, J = 1.2 Hz, 3H), |
| | | | | | 2.38-2.30 (m, 1H), 2.25 (dd, J = 9.4, 4.8 Hz, |
| | | | | | 1H), 2.15 (s, 3H), |
| | | | | | 2.08-1.97 (m, 1H), 1.66-1.56 (m, |
| | | | | | 1H). |
| 152 | 152 | 406.46 | N-(4- | 98.9% | ¹H NMR (400 MHz, |
| | | | methanesulfonylpyridin- | [M + H]⁺ = 407.1 | DMSO) δ 8.99 (s, 1H), 8.82 (d, |
| | | | 3-yl)-8-(4- | | J = 1.9 Hz, 1H), 8.72 (d, J = 1.9 Hz, |
| | | | methoxyphenyl)quinoxalin- | | 1H), 8.57 (d, J = 5.1 Hz, |
| | | | 6-amine | | 1H), 8.44 (s, 1H), 7.85 (dd, J = 5.1, |
| | | | | | 0.5 Hz, 1H), 7.78 (d, J = 2.6 Hz, |
| | | | | | 1H), 7.66-7.60 (m, 2H), |
| | | | | | 7.55 (d, J = 2.6 Hz, 1H), |
| | | | | | 7.10-7.03 (m, 2H), 3.83 (s, 3H), |
| | | | | | 3.37 (s, 3H). |
| 153 | 153 | 420.49 | N-(4- | 98.6% | ¹H NMR (400 MHz, |
| | | | methanesulfonylpyridin- | [M + H]⁺ = 421.1 | DMSO) δ 8.97 (s, 1H), 8.81 (d, |
| | | | 3-yl)-8-(5- | | J = 1.9 Hz, 1H), 8.66 (d, J = 1.8 Hz, |
| | | | methoxy-2- | | 1H), 8.56 (d, J = 5.1 Hz, |
| | | | methylphenyl)quinoxalin- | | 1H), 8.46 (s, 1H), 7.84 (dd, J = 5.1, |
| | | | 6-amine | | 0.5 Hz, 1H), 7.63 (d, J = 2.6 Hz, |
| | | | | | 1H), 7.61 (d, J = 2.6 Hz, |
| | | | | | 1H), 7.22 (d, J = 8.7 Hz, 1H), |
| | | | | | 6.92 (dd, J = 8.4, 2.8 Hz, 1H), |
| | | | | | 6.84 (d, J = 2.8 Hz, 1H), 3.74 (s, |
| | | | | | 3H), 3.37 (s, 3H), 1.92 (s, 3H). |
| 154 | 154 | 465.48 | 8-[1- | 99.1% | ¹H NMR (400 MHz, |
| | | | (difluoromethyl)- | [M + H]⁺ = 466.1 | DMSO) δ 9.02 (s, 1H), 8.83 (d, |
| | | | 1H-indol-6-yl]-N- | | J = 1.8 Hz, 1H), 8.73 (d, J = 1.8 Hz, |
| | | | (4- | | 1H), 8.58 (d, J = 5.1 Hz, |
| | | | methanesulfonylpyridin- | | 1H), 8.49 (s, 1H), 8.06 (t, J = 59.5 Hz, |
| | | | 3- | | 1H), 7.99 (s, 1H), |
| | | | yl)quinoxalin-6- | | 7.86 (dd, J = 5.1, 0.6 Hz, 1H), |
| | | | amine | | 7.86 (d, J = 5.1 Hz, 1H), 7.76 (d, J = 8.2 Hz, |
| | | | | | 1H), 7.72 (d, J = 3.5 Hz, |
| | | | | | 1H), 7.56 (d, J = 2.5 Hz, 1H), |
| | | | | | 7.51 (dd, J = 8.2, 1.3 Hz, 1H), |
| | | | | | 6.78 (dd, J = 3.5, 0.8 Hz, 1H), |
| | | | | | 3.38 (s, 3H). |
| 157 | 157 | 455.33 | 8-(4- | 98.6% | ¹H NMR (400 MHz, |
| | | | bromophenyl)-N- | [M + H]⁺ = 455.1 | DMSO) δ 8.99 (s, 1H), 8.84 (d, |
| | | | (4- | | J = 1.8 Hz, 1H), 8.73 (d, J = 1.8 Hz, |
| | | | methanesulfonylpyridin- | | 1H), 8.58 (d, J = 5.1 Hz, |
| | | | 3- | | 1H), 8.48 (s, 1H), 7.85 (dd, J = 5.1, |
| | | | yl)quinoxalin-6- | | 0.4 Hz, 1H), 7.83 (d, J = 2.6 Hz, |
| | | | amine | | 1H), 7.73-7.69 (m, 2H), |
| | | | | | 7.67-7.61 (m, 2H), 7.60 (d, J = 2.6 Hz, |
| | | | | | 1H), 3.37 (s, 3H). |
| 158 | 158 | 455.33 | 8-(3- | 100.0% | ¹H NMR (400 MHz, |
| | | | bromophenyl)-N- | [M + H]⁺ = 455.1 | DMSO) δ 9.00 (s, 1H), 8.85 (d, |
| | | | (4- | | J = 1.8 Hz, 1H), 8.75 (dd, J = 1.9, |
| | | | methanesulfonylpyridin- | | 0.4 Hz, 1H), 8.58 (d, J = 5.1 Hz, |
| | | | 3- | | 1H), 8.47 (s, 1H), |
| | | | yl)quinoxalin-6- | | 7.88-7.83 (m, 3H), 7.71-7.63 (m, |
| | | | amine | | 2H), 7.62 (d, J = 2.6 Hz, 1H), |
| | | | | | 7.48 (t, J = 7.9 Hz, 1H), 3.37 (s, |
| | | | | | 3H). |
| 160 | 160 | 488.51 | 2-aminopyrimidin- | 91.3% | ¹H NMR (400 MHz, |
| | | | 4-yl 3-{[8-(1- | [M + H]⁺ = 489.2 | DMSO) δ 12.69 (s, 2H), |
| | | | methyl-1H-indol- | | 10.23 (s, 1H), 8.96 (s, 1H), 8.81 (d, J = 1.8 Hz, |
| | | | 6-yl)quinoxalin-6- | | 1H), 8.71 (d, J = 1.8 Hz, |
| | | | yl]amino}pyridine- | | 1H), 8.24 (d, J = 5.0 Hz, 1H), |
| | | | 4-carboxylate | | 7.93 (s, 1H), 7.85 (d, J = 4.3 Hz, |
| | | | | | 1H), 7.76 (s, 1H), 7.73 (s, 1H), |
| | | | | | 7.72 (d, J = 7.6 Hz, 1H), |
| | | | | | 7.64 (dd, J = 8.2, 0.7 Hz, 1H), |
| | | | | | 7.40 (d, J = 3.1 Hz, 1H), 7.34 (dd, J = 8.2, |
| | | | | | 1.3 Hz, 1H), 6.49 (dd, J = 3.1, |
| | | | | | 0.9 Hz, 1H), 6.00 (d, J = 7.5 Hz, |
| | | | | | 1H), 3.83 (s, 3H). |
| 161 | 161 | 433.50 | 8-(1,2- | 97.6% | ¹H NMR (400 MHz, |
| | | | benzothiazol-5-yl)- | [M + H]⁺ = 434.1 | DMSO) δ 9.23 (d, J = 0.9 Hz, |
| | | | N-(4- | | 1H), 9.02 (s, 1H), 8.86 (d, J = 1.9 Hz, |
| | | | methanesulfonylpyridin- | | 1H), 8.75 (d, J = 1.9 Hz, |
| | | | 3- | | 1H), 8.58 (d, J = 5.1 Hz, 1H), |
| | | | yl)quinoxalin-6- | | 8.51 (s, 1H), 8.49 (dd, J = 1.5, |
| | | | amine | | 0.7 Hz, 1H), 8.34 (dt, J = 8.5, |
| | | | | | 0.7 Hz, 1H), 7.94 (d, J = 2.6 Hz, |
| | | | | | 1H), 7.92 (dd, J = 8.5, 1.6 Hz, |
| | | | | | 1H), 7.86 (dd, J = 5.1, 0.5 Hz, |
| | | | | | 1H), 7.64 (d, J = 2.6 Hz, 1H), |
| | | | | | 3.39 (s, 3H). |
| 162 | 162 | 448.52 | 8-(2-amino-1,3- | 99.6% | ¹H NMR (400 MHz, |
| | | | benzothiazol-5-yl)- | [M + H]⁺ = 449.1 | DMSO) δ 9.00 (s, 1H), 8.83 (d, |
| | | | N-(4- | | J = 1.8 Hz, 1H), 8.73 (d, J = 1.9 Hz, |
| | | | methanesulfonylpyridin- | | 1H), 8.57 (d, J = 5.1 Hz, |
| | | | 3- | | 1H), 8.46 (s, 1H), 7.85 (d, J = 5.1 Hz, |
| | | | yl)quinoxalin-6- | | 1H), 7.83 (d, J = 2.6 Hz, |
| | | | amine | | 1H), 7.76 (d, J = 8.1 Hz, 1H), |
| | | | | | 7.62 (d, J = 1.6 Hz, 1H), |
| | | | | | 7.57 (d, J = 2.6 Hz, 1H), 7.53 (s, 2H), |
| | | | | | 7.31 (dd, J = 8.1, 1.7 Hz, 1H), |
| | | | | | 3.38 (s, 3H). |
| 163 | 163 | 460.43 | N-(4- | 99.9% | ¹H NMR (400 MHz, |
| | | | methanesulfonylpyridin- | [M + H]⁺ = 459.2 | DMSO) δ 9.01 (s, 1H), 8.85 (d, |
| | | | 3-yl)-8-[3- | | J = 1.9 Hz, 1H), 8.74 (d, J = 1.8 Hz, |
| | | | (trifluoromethoxy)phenyl]quinoxalin- | | 1H), 8.58 (d, J = 5.1 Hz, |
| | | | 6-amine | | 1H), 7.89 (d, J = 2.6 Hz, 1H), |
| | | | | | 7.85 (dd, J = 5.1, 0.6 Hz, 1H), |
| | | | | | 8.49 (s, 1H), 7.72 (dt, J = 7.9, |
| | | | | | 1.3 Hz, 1H), 7.69-7.67 (m, |
| | | | | | 1H), 7.66 (d, J = 8.2 Hz, 1H), |
| | | | | | 7.62 (d, J = 2.6 Hz, 1H), |
| | | | | | 7.46 (ddt, J = 8.1, 2.4, 1.0 Hz, 1H), |
| | | | | | 3.38 (s, 3H). |
| 164 | 164 | 488.57 | N-(4-{7-[(4- | 98.6% | ¹H NMR (400 MHz, |
| | | | methanesulfonylpyridin- | [M + H]⁺ = 489.2 | DMSO) δ 10.08 (s, 1H), 8.99 (s, |
| | | | 3- | | 1H), 8.82 (d, J = 1.8 Hz, 1H), |
| | | | yl)amino]quinoxalin- | | 8.73 (d, J = 1.8 Hz, 1H), |
| | | | 5- | | 8.57 (d, J = 5.1 Hz, 1H), 8.45 (s, 1H), |
| | | | yl}phenyl)pyrrolidine- | | 7.85 (d, J = 5.1 Hz, 1H), |
| | | | 2-carboxamide | | 7.80 (d, J = 1.7 Hz, 1H), 7.79 (d, J = 13.0 Hz, |
| | | | | | 2H), 7.66-7.61 (m, |
| | | | | | 1H), 7.55 (d, J = 2.5 Hz, 1H), |
| | | | | | 3.74 (dd, J = 8.8, 5.7 Hz, 1H), |
| | | | | | 3.38 (s, 3H), 3.34 (s, 1H), |
| | | | | | 2.92 (t, J = 6.6 Hz, 2H), |
| | | | | | 2.14-2.02 (m, 1H), 1.81 (ddd, J = 12.5, |
| | | | | | 9.9, 6.6 Hz, 1H), 1.68 (p, J = 6.7 Hz, |
| | | | | | 2H). |
| 165 | 165 | 488.57 | N-(3-{7-[(4- | 97.6% | ¹H NMR (400 MHz, |
| | | | methanesulfonylpyridin- | [M + H]⁺ = 489.2 | DMSO) δ 10.06 (s, 1H), 8.99 (s, |
| | | | 3- | | 1H), 8.83 (d, J = 1.6 Hz, 1H), |
| | | | yl)amino]quinoxalin- | | 8.73 (d, J = 1.7 Hz, 1H), |
| | | | 5- | | 8.58 (d, J = 5.0 Hz, 1H), 8.48 (s, 1H), |
| | | | yl}phenyl)pyrrolidine- | | 7.93 (s, 1H), 7.85 (d, J = 5.0 Hz, |
| | | | 2-carboxamide | | 1H), 7.80 (d, J = 2.3 Hz, 1H), |
| | | | | | 7.75 (d, J = 8.1 Hz, 1H), |
| | | | | | 7.56 (d, J = 2.5 Hz, 1H), 7.43 (t, J = 7.8 Hz, |
| | | | | | 1H), 7.33 (d, J = 7.7 Hz, |
| | | | | | 1H), 3.72 (dd, J = 8.7, 5.8 Hz, |
| | | | | | 1H), 3.38 (s, 3H), 3.33 (s, 1H), |
| | | | | | 2.90 (t, J = 6.6 Hz, 2H), |
| | | | | | 2.06 (ddd, J = 15.5, 12.9, 7.6 Hz, |
| | | | | | 1H), 1.79 (dt, J = 12.6, 6.7 Hz, |
| | | | | | 1H), 1.66 (p, J = 6.9 Hz, 1H). |
| 166 | 166 | 443.53 | 8-(1-ethyl-1H- | 92.7% | ¹H NMR (400 MHz, |
| | | | indol-6-yl)-N-(4- | [M + H]⁺ = 442.2 | DMSO) δ 9.02 (s, 1H), 8.81 (d, |
| | | | methanesulfonylpyridin- | | J = 1.8 Hz, 1H), 8.72 (d, J = 1.8 Hz, |
| | | | 3- | | 1H), 8.57 (d, J = 5.1 Hz, |
| | | | yl)quinoxalin-6- | | 1H), 8.46 (s, 1H), 7.87 (d, J = 2.6 Hz, |
| | | | amine | | 1H), 7.85 (d, J = 5.1 Hz, |
| | | | | | 1H), 7.76 (s, 1H), 7.64 (d, J = 8.2 Hz, |
| | | | | | 1H), 7.55 (d, J = 2.6 Hz, |
| | | | | | 1H), 7.47 (d, J = 3.1 Hz, 1H), |
| | | | | | 7.33 (dd, J = 8.2, 1.4 Hz, 1H), |
| | | | | | 6.50 (dd, J = 3.1, 0.8 Hz, 1H), |
| | | | | | 4.26 (q, J = 7.2 Hz, 2H), 3.39 (s, |
| | | | | | 3H), 1.40 (t, J = 7.2 Hz, 3H). |
| 167 | 167 | 431.47 | N-(4- | 99.5% | ¹H NMR (400 MHz, |
| | | | methanesulfonylpyridin- | [M + H]⁺ = 432.0 | DMSO) δ 9.02 (s, 1H), 8.85 (d, |
| | | | 3-yl)-8-(1- | | J = 1.8 Hz, 1H), 8.73 (d, J = 1.8 Hz, |
| | | | methyl-1H-1,2,3- | | 1H), 8.57 (d, J = 5.1 Hz, |
| | | | benzotriazol-5- | | 1H), 8.48 (s, 1H), 8.30 (dd, J = 1.5, |
| | | | yl)quinoxalin-6- | | 0.8 Hz, 1H), 7.95 (dd, J = 8.7, |
| | | | amine | | 0.8 Hz, 2H), 7.94 (d, J = 1.9 Hz, |
| | | | | | 1H), 7.88 (dd, J = 8.6, 1.5 Hz, |
| | | | | | 1H), 7.85 (dd, J = 5.1, 0.6 Hz, |
| | | | | | 1H), 7.63 (d, J = 2.5 Hz, |
| | | | | | 1H), 4.37 (s, 3H), 3.38 (s, 3H). |
| 168 | 168 | 526.66 | 2-{[8-(1-methyl- | 98.4% | ¹H NMR (400 MHz, |
| | | | 1H-indol-6- | [M + H]⁺ = 527.3 | DMSO) δ 8.80 (d, J = 1.8 Hz, |
| | | | yl)quinoxalin-6- | | 1H), 8.70 (d, J = 1.9 Hz, 1H), |
| | | | yl]amino}-N-[(1- | | 8.08 (s, 1H), 7.86 (dd, J = 8.0, |
| | | | methylpyrrolidin- | | 1.5 Hz, 1H), 7.77 (dd, J = 8.4, |
| | | | 3- | | 1.0 Hz, 2H), 7.76 (d, J = 2.8 Hz, |
| | | | yl)methyl]benzene- | | 1H), 7.72 (dt, J = 1.6, 0.8 Hz, |
| | | | 1-sulfonamide | | 1H), 7.66-7.64 (ddd, J = 8.3, |
| | | | | | 7.3, 1.6 Hz, 1H), 7.64 (dd, J = 8.2, |
| | | | | | 0.7 Hz, 1H), 7.58 (d, J = 2.6 Hz, |
| | | | | | 1H), 7.40 (d, J = 3.1 Hz, |
| | | | | | 1H), 7.34 (dd, J = 8.2, 1.4 Hz, |
| | | | | | 1H), 7.21 (ddd, J = 8.1, 7.3, 1.1 Hz, |
| | | | | | 1H), 6.49 (dd, J = 3.1, 0.7 Hz, |
| | | | | | 1H), 4.27-3.94 (m, 1H), |
| | | | | | 3.83 (s, 3H), 2.75 (d, J = 7.3 Hz, |
| | | | | | 2H), 2.30-2.05 (m, 4H), |
| | | | | | 2.01 (s, 3H), 2.01-1.95 (m, 1H), |
| | | | | | 1.72-1.60 (m, 1H), |
| | | | | | 1.25-1.13 (m, 1H). |
| 169 | 169 | 447.53 | N-(4- | 99.8% | ¹H NMR (400 MHz, |
| | | | methanesulfonylpyridin- | [M + H]⁺ = 448.2 | DMSO) δ 9.02 (s, 1H), 8.85 (d, |
| | | | 3-yl)-8-(2- | | J = 1.8 Hz, 1H), 8.74 (d, J = 1.8 Hz, |
| | | | methyl-1,3- | | 1H), 8.58 (d, J = 5.1 Hz, |
| | | | benzothiazol-5- | | 1H), 8.49 (s, 1H), 8.20 (dd, J = 1.7, |
| | | | yl)quinoxalin-6- | | 0.6 Hz, 1H), 8.14 (dd, J = 8.2, |
| | | | amine | | 0.6 Hz, 1H), 7.91 (d, J = 2.5 Hz, |
| | | | | | 1H), 7.85 (dd, J = 5.1, 0.6 Hz, |
| | | | | | 1H), 7.70 (dd, J = 8.3, 1.7 Hz, |
| | | | | | 1H), 7.62 (d, J = 2.5 Hz, |
| | | | | | 1H), 3.38 (s, 3H), 2.85 (s, 3H). |
| 170 | 170 | 431.47 | N-(4- | 100.0% | ¹H NMR (400 MHz, |
| | | | methanesulfonylpyridin- | [M + H]⁺ = 432.0 | DMSO) δ 9.01 (s, 1H), 8.86 (d, |
| | | | 3-yl)-8-(1- | | J = 1.8 Hz, 1H), 8.74 (d, J = 1.8 Hz, |
| | | | methyl-1H-1,2,3- | | 1H), 8.58 (d, J = 5.1 Hz, |
| | | | benzotriazol-6- | | 1H), 8.51 (s, 1H), 8.12 (dd, J = 10.1, |
| | | | yl)quinoxalin-6- | | 0.8 Hz, 1H), 8.11 (m, 1H), |
| | | | amine | | 7.92 (d, J = 2.5 Hz, 1H), |
| | | | | | 7.86 (d, J = 5.0 Hz, 1H), 7.69 (dd, J = 8.6, |
| | | | | | 1.5 Hz, 1H), 7.63 (d, J = 2.5 Hz, |
| | | | | | 1H), 4.36 (s, 3H), 3.39 (s, |
| | | | | | 3H). |
| 171 | 171 | 512.63 | 2-{[8-(1-methyl- | 93.8% | ¹H NMR (400 MHz, |
| | | | 1H-indol-6- | [M + H]⁺ = 513.2 | DMSO) δ 8.82 (d, J = 1.8 Hz, |
| | | | yl)quinoxalin-6- | | 1H), 8.72 (d, J = 1.8 Hz, 1H), |
| | | | yl]amino}-N-(1- | | 8.19 (d, J = 8.1 Hz, 1H), |
| | | | methylpyrrolidin- | | 7.88 (dd, J = 7.9, 1.5 Hz, 1H), |
| | | | 3-yl)benzene-1- | | 7.75 (m, 3H), 7.63 (m, 3H), 7.41 (d, |
| | | | sulfonamide | | J = 3.0 Hz, 1H), 7.35 (dd, J = 8.2, |
| | | | | | 1.4 Hz, 1H), 7.18 (ddd, J = 8.2, |
| | | | | | 7.3, 1.2 Hz, 1H), 6.49 (dd, |
| | | | | | J = 3.1, 0.9 Hz, 1H), 3.84 (s, |
| | | | | | 3H), 3.66 (m, 1H), 2.37 (m, |
| | | | | | 2H), 2.26-2.12 (m, 2H), |
| | | | | | 2.07 (s, 3H), 1.94-1.81 (m, 1H), |
| | | | | | 1.50-1.38 (m, 1H). |
| 172 | 172 | 527.64 | 2-{[8-(1-methyl- | 96.9% | ¹H NMR (400 MHz, DMSO-d₆) |
| | | | 1H-indol-6- | [M + H]⁺ = 528.2 | δ 8.81 (d, J = 1.9 Hz, 1H), |
| | | | yl)quinoxalin-6- | | 8.72 (d, J = 1.8 Hz, 1H), 8.11 (s, 1H), |
| | | | yl]amino}-N- | | 7.94 (td, J = 5.1, 0.6 Hz, 1H), |
| | | | [(oxan-4- | | 7.88 (dd, J = 7.9, 1.5 Hz, 1H), |
| | | | yl)methyl]benzene- | | 7.77 (dd, J = 8.2, 0.7 Hz, 1H), |
| | | | 1-sulfonamide | | 7.75 (d, J = 2.6 Hz, 1H), 7.72 (s, |
| | | | | | 1H), 7.64 (d, J = 8.2 Hz, 1H), |
| | | | | | 7.64 (ddd, J = 8.4, 7.5, 1.5 Hz, |
| | | | | | 1H), 7.58 (d, J = 2.6 Hz, 1H), |
| | | | | | 7.41 (d, J = 3.1 Hz, 1H), |
| | | | | | 7.35 (dd, J = 8.2, 1.5 Hz, 1H), |
| | | | | | 7.23-7.18 (m, 1H), 6.49 (dd, J = 3.0, |
| | | | | | 0.6 Hz, 1H), 3.84 (s, 3H), |
| | | | | | 3.66 (dd, J = 11.5, 2.9 Hz, 2H), |
| | | | | | 3.05 (td, J = 11.7, 1.7 Hz, 2H), |
| | | | | | 2.71 (t, J = 5.9 Hz, 2H), 1.42 (d, J = 13.3 Hz, |
| | | | | | 1H), 1.26 (d, J = 6.9 Hz, |
| | | | | | 1H), 1.14 (d, J = 6.7 Hz, |
| | | | | | 1H), 0.96 (dd, J = 11.8, 3.6 Hz, |
| | | | | | 1H), 0.87 (d, J = 6.7 Hz, 1H). |
| 173 | 173 | 523.62 | 2-{[8-(-methyl- | 97.7% | ¹H NMR (400 MHz, |
| | | | 1H-indol-6- | [M + H]⁺ = 524.2 | DMSO) δ 8.81 (d, J = 1.8 Hz, |
| | | | yl)quinoxalin-6- | | 1H), 8.71 (d, J = 1.8 Hz, 1H), |
| | | | yl]amino}-N-[(1- | | 8.14 (t, J = 5.8 Hz, 1H), 8.02 (s, |
| | | | methyl-1H- | | 1H), 7.86 (dd, J = 7.9, 1.4 Hz, |
| | | | pyrazol-4- | | 1H), 7.75-7.67 (m, 3H), |
| | | | yl)methyl]benzene- | | 7.64 (d, J = 8.3 Hz, 1H), |
| | | | 1-sulfonamide | | 7.63-7.58 (m, 1H), 7.51 (d, J = 2.5 Hz, |
| | | | | | 1H), 7.40 (d, J = 3.0 Hz, 1H), |
| | | | | | 7.35 (dd, J = 8.2, 1.3 Hz, 1H), |
| | | | | | 7.28 (s, 1H), 7.20-7.12 (m, |
| | | | | | 1H), 7.02 (s, 1H), 6.49 (dd, J = 3.1, |
| | | | | | 0.8 Hz, 1H), 3.92 (d, J = 5.7 Hz, |
| | | | | | 2H), 3.84 (s, 3H), 3.61 (s. |
| | | | | | 3H). |
| 174 | 174 | 448.52 | 8-(2-amino-1,3- | 100.0% | ¹H NMR (400 MHz, |
| | | | benzothiazol-6-yl)- | [M + H]⁺ = 449.2 | DMSO) δ 8.99 (s, 1H), 8.83 (d, |
| | | | N-(4- | | J = 1.8 Hz, 1H), 8.74 (d, J = 1.8 Hz, |
| | | | methanesulfonylpyridin- | | 1H), 8.57 (d, J = 5.1 Hz, |
| | | | 3- | | 1H), 8.43 (s, 1H), 7.97 (d, J = 1.7 Hz, |
| | | | yl)quinoxalin-6- | | 1H), 7.85 (d, J = 5.1 Hz, |
| | | | amine | | 1H), 7.83 (d, J = 2.6 Hz, 1H), |
| | | | | | 7.58 (s, 2H), 7.58-7.56 (m, |
| | | | | | 1H), 7.53 (dd, J = 8.3, 1.8 Hz, |
| | | | | | 1H), 7.44 (d, J = 8.3 Hz, 1H), |
| | | | | | 3.38 (s, 3H). |
| 175 | 175 | 408.51 | N-{4- | 97.1% | ¹H NMR (400 MHz, |
| | | | [(dimethylamino)methyl]pyridin- | [M + H]⁺ = 409.3 | DMSO) δ 8.82 (s, 1H), 8.74 (s, |
| | | | 3- | | 1H), 8.73 (d, J = 1.8 Hz, 1H), |
| | | | yl}-8-(1-methyl- | | 8.61 (d, J = 1.8 Hz, 1H), |
| | | | 1H-indol-6- | | 8.33 (d, J = 4.8 Hz, 1H), |
| | | | yl)quinoxalin-6- | | 7.70-7.69 (m, 1H), 7.63 (d, J = 8.2 Hz, |
| | | | amine | | 1H), 7.59 (d, J = 2.6 Hz, 1H), |
| | | | | | 7.46 (d, J = 4.8 Hz, 1H), |
| | | | | | 7.40 (d, J = 3.1 Hz, 1H), 7.31 (dd, J = 8.2, |
| | | | | | 1.3 Hz, 1H), 7.15 (d, J = 2.6 Hz, |
| | | | | | 1H), 6.49 (dd, J = 3.0, 0.8 Hz, |
| | | | | | 1H), 3.83 (s, 3H), 3.54 (s, |
| | | | | | 2H), 2.22 (s, 6H). |
| 176 | 176 | 407.52 | N-{2- | 99.9% | ¹H NMR (400 MHz, |
| | | | [(dimethylamino)methyl]phenyl}- | [M + H]⁺ = 408.4 | DMSO) δ 9.03 (s, 1H), 8.72 (d, |
| | | | 8- | | J = 1.9 Hz, 1H), 8.59 (d, J = 1.9 Hz, |
| | | | (1-methyl-1H- | | 1H), 7.70 (dd, J = 1.6, 0.8 Hz, |
| | | | indol-6- | | 1H), 7.63 (dd, J = 8.2, 0.7 Hz, |
| | | | yl)quinoxalin-6- | | 1H), 7.59-7.55 (m, 2H), |
| | | | amine | | 7.40 (d, J = 3.1 Hz, 1H), |
| | | | | | 7.37-7.34 (m, 2H), 7.32 (dd, J = 8.2, |
| | | | | | 1.5 Hz, 1H), 7.32 (dd, J = 8.2, |
| | | | | | 1.5 Hz, 1H), 7.07 (td, J = 7.5, |
| | | | | | 1.2 Hz, 1H), 6.49 (dd, J = 3.1, |
| | | | | | 0.9 Hz, 1H), 3.84 (s, 3H), |
| | | | | | 3.51 (s, 2H), 2.21 (s, 5H), 1.24 (s, |
| | | | | | 2H). |
| 178 | 178 | 394.43 | 2-{[8-(1-methyl- | 94.2% | ¹H NMR (400 MHz, DMSO-d₆) |
| | | | 1H-indol-6- | [M + H]⁺ = 395.2 | δ 13.02 (s, 1H), 8.75 (d, J = 1.9 Hz, |
| | | | yl)quinoxalin-6- | | 1H), 8.61 (d, J = 1.9 Hz, |
| | | | yl]amino}benzoic | | 1H), 8.01 (dd, J = 7.7, 1.5 Hz, |
| | | | acid | | 1H), 7.72 (s, 1H), 7.67 (d, J = 2.5 Hz, |
| | | | | | 1H), 7.62 (t, J = 8.2 Hz, |
| | | | | | 2H), 7.54 (d, J = 2.7 Hz, 1H), |
| | | | | | 7.40 (d, J = 3.1 Hz, 1H), |
| | | | | | 7.34 (dd, J = 8.1, 1.1 Hz, 1H), |
| | | | | | 7.32 (dd, J = 7.6, 1.8 Hz, 1H), |
| | | | | | 6.85 (td, J = 8.2, 7.7, 0.9 Hz, 1H), |
| | | | | | 6.49 (dd, J = 3.1, 0.8 Hz, 1H), |
| | | | | | 3.84 (s, 3H). |
| 179 | 179 | 395.42 | 3-{[8-(1-methyl- | 96.0% | ¹H NMR (400 MHz, |
| | | | 1H-indol-6- | [M + H]⁺ = 396.2 | DMSO) δ 12.53 (s, 1H), 8.92 (s, |
| | | | yl)quinoxalin-6- | | 1H), 8.78 (d, J = 1.8 Hz, 1H), |
| | | | yl]amino}pyridine- | | 8.65 (d, J = 1.9 Hz, 1H), |
| | | | 4-carboxylic acid | | 8.08 (d, J = 4.8 Hz, 1H), 7.77 (dd, J = 4.8, |
| | | | | | 0.6 Hz, 1H), 7.74 (dt, J = 1.5, |
| | | | | | 0.8 Hz, 1H), 7.67 (d, J = 2.6 Hz, |
| | | | | | 1H), 7.63 (dd, J = 8.2, 0.7 Hz, |
| | | | | | 1H), 7.58 (d, J = 2.6 Hz, |
| | | | | | 1H), 7.39 (d, J = 3.1 Hz, 1H), |
| | | | | | 7.34 (dd, J = 8.2, 1.5 Hz, 1H), |
| | | | | | 6.49 (dd, J = 3.0, 0.7 Hz, 1H), |
| | | | | | 3.84 (s, 3H). |
| 181 | 181 | 463.55 | 3-{[8-(1-methyl- | 84.6% | ¹H NMR (400 MHz, |
| | | | 1H-indol-6- | [M + H]⁺ = 464.3 | DMSO) δ 9.30 (s, 1H), 9.10 (d, |
| | | | yl)quinoxalin-6- | | J = 6.9 Hz, 1H), 8.86 (s, 1H), |
| | | | yl]amino}-N-(1- | | 8.78 (d, J = 1.8 Hz, 1H), |
| | | | methylazetidin-3- | | 8.67 (d, J = 1.8 Hz, 1H), 8.34 (d, J = 5.0 Hz, |
| | | | yl)pyridine-4- | | 1H), 8.23 (s, 1H), |
| | | | carboxamide | | 7.71 (s, 1H), 7.66 (d, J = 2.6 Hz, 1H), |
| | | | | | 7.62 (d, J = 8.1 Hz, 1H), |
| | | | | | 7.59 (d, J = 5.0 Hz, 1H), 7.45 (d, J = 2.6 Hz, |
| | | | | | 1H), 7.40 (d, J = 3.0 Hz, |
| | | | | | 1H), 7.33 (dd, J = 8.2, 1.3 Hz, |
| | | | | | 1H), 6.48 (d, J = 3.0 Hz, 1H), |
| | | | | | 4.32 (h, J = 7.2 Hz, 1H), 3.83 (s, |
| | | | | | 3H), 3.44 (dd, 2H), 2.82 (dd, J = 7.4 Hz, |
| | | | | | 2H), 2.14 (s, 3H). |
| 182 | 182 | 491.60 | N-methyl-3-{[8-(1- | 96.0% | ¹H NMR (400 MHz, DMSO-d₆) |
| | | | methyl-1H-indol- | [M + H]⁺ = 492.3 | δ 8.80 (d, J = 10.2 Hz, 1H), |
| | | | 6-yl)quinoxalin-6- | | 8.76-8.74 (m, 2H), 8.67 (d, J = 37.1 Hz, |
| | | | yl]amino}-N-(1- | | 1H), 8.63 (t, J = 1.9 Hz, |
| | | | methylpyrrolidin- | | 1H), 8.41 (t, J = 4.9 Hz, 1H), |
| | | | 3-yl)pyridine-4- | | 7.69 (d, J = 6.9 Hz, 1H), |
| | | | carboxamide | | 7.63 (d, J = 8.2 Hz, 1H), 7.62 (dd, J = 38.0, |
| | | | | | 2.6 Hz, 1H), 7.40 (d, J = 3.1 Hz, |
| | | | | | 1H), 7.36 (dd, J = 21.4, |
| | | | | | 5.0 Hz, 1H), 7.33-7.29 (m, |
| | | | | | 1H), 7.26 (dd, J = 37.3, 2.5 Hz, |
| | | | | | 1H), 6.49 (dd, J = 3.1, 0.8 Hz, |
| | | | | | 1H), 4.89 (tt, J = 9.6, 4.9 Hz, |
| | | | | | 0.5H), 4.18-4.09 (m, 0.5H), |
| | | | | | 3.83 (s, 3H), 2.88 (s, 1.5H), |
| | | | | | 2.76 (s, 1.5H), 2.65-2.56 (m, |
| | | | | | 1H), 2.09 (s, 1.5H), 2.07 (s, |
| | | | | | 1.5H), 2.04-1.94 (m, 2H), |
| | | | | | 1.89-1.64 (m, 1H), |
| | | | | | 1.57-1.44 (m, 1H). |
| 183 | 183 | 476.58 | 2-{[8-(1-methyl- | 92.8% | ¹H NMR (400 MHz, DMSO) δ |
| | | | 1H-indol-6- | [M + H]⁺ = 477.3 | 9.77 (s, 1H), 8.76 (d, J = 1.8 Hz, |
| | | | yl)quinoxalin-6- | | 1H), 8.65 (d, J = 1.9 Hz, 1H), |
| | | | yl]amino}-N-(1- | | 8.61 (d, J = 7.1 Hz, 1H), |
| | | | methylpyrrolidin- | | 7.73 (dd, J = 7.2, 2.0 Hz, 1H), |
| | | | 3-yl)benzamide | | 7.72 (s, 1H), 7.64 (t, J = 8.4 Hz, 2H), |
| | | | | | 7.65 (s, 1H), 7.52 (d, J = 2.6 Hz, |
| | | | | | 1H), 7.50 (td, J = 8.2, 7.3, 1.3 Hz, |
| | | | | | 1H), 7.40 (d, J = 3.1 Hz, 1H), |
| | | | | | 7.34 (dd, J = 8.2, 1.4 Hz, 1H), |
| | | | | | 7.08 (ddd, J = 8.0, 7.3, 1.1 Hz, |
| | | | | | 1H), 6.48 (d, J = 3.1 Hz, 1H), |
| | | | | | 4.39-4.30 (m, 1H), 3.84 (s, 3H), |
| | | | | | 2.65 (dd, J = 9.4, 7.1 Hz, 1H), |
| | | | | | 2.55-2.51 (m, 1H), |
| | | | | | 2.40-2.29 (m, 2H), 2.19 (s, 3H), |
| | | | | | 2.14-2.03 (m, 1H), 1.75-1.65 (m, J = 13.0, |
| | | | | | 5.5 Hz, 1H) |
| 184 | 184 | 457.55 | N-(4- | 90.2% | ¹H NMR (400 MHz, |
| | | | methanesulfonylpyridin- | [M + H]⁺ = 456.3 | DMSO) δ 9.01 (s, 1H), 8.80 (d, |
| | | | 3-yl)-8-(1- | | J = 1.8 Hz, 1H), 8.71 (d, J = 1.8 Hz, |
| | | | propyl-1H-indol-6- | | 1H), 8.57 (d, J = 5.1 Hz, |
| | | | yl)quinoxalin-6- | | 1H), 8.46 (s, 1H), 7.85 (s, 1H), |
| | | | amine | | 7.84 (d, J = 1.6 Hz, 1H), 7.75 (s, |
| | | | | | 1H), 7.63 (d, J = 8.2 Hz, 1H), |
| | | | | | 7.53 (d, J = 2.6 Hz, 1H), |
| | | | | | 7.45 (d, J = 3.1 Hz, 1H), 7.32 (dd, J = 8.2, |
| | | | | | 1.4 Hz, 1H), 6.49 (dd, J = 3.0, |
| | | | | | 0.5 Hz, 1H), 4.18 (t, J = 6.9 Hz, |
| | | | | | 2H), 3.38 (s, 3H), |
| | | | | | 1.87-1.74 (m, 2H), 0.85 (t, J = 7.4 Hz, |
| | | | | | 3H). |
| 185 | 185 | 444.43 | N-(4- | 99.9% | ¹H NMR (400 MHz, |
| | | | methanesulfonylpyridin- | [M + H]⁺ = 445.2 | DMSO) δ 9.00 (s, 1H), 8.86 (d, |
| | | | 3-yl)-8-[4- | | J = 1.9 Hz, 1H), 8.74 (d, J = 1.8 Hz, |
| | | | (trifluoromethyl)phenyl]quinoxalin- | | 1H), 8.59 (d, J = 5.1 Hz, |
| | | | 6-amine | | 1H), 8.51 (s, 1H), |
| | | | | | 7.92-7.84 (m, 6H), 7.64 (d, J = 2.6 Hz, |
| | | | | | 1H), 3.37 (s, 3H). |
| 186 | 186 | 409.44 | 8-(4-amino-3- | 99.9% | ¹H NMR (400 MHz, |
| | | | fluorophenyl)-N- | [M + H]⁺ = 410.1 | DMSO) δ 8.97 (s, 1H), 8.81 (d, |
| | | | (4- | | J = 1.8 Hz, 1H), 8.73 (d, J = 1.8 Hz, |
| | | | methanesulfonylpyridin- | | 1H), 8.56 (d, J = 5.1 Hz, |
| | | | 3- | | 1H), 8.56 (d, J = 5.1 Hz, |
| | | | yl)quinoxalin-6- | | 1H), 8.39 (s, 1H), 7.84 (d, J = 5.1 Hz, |
| | | | amine | | 1H), 7.77 (d, J = 2.6 Hz, |
| | | | | | 1H), 7.51 (d, J = 2.5 Hz, 1H), |
| | | | | | 7.43 (dd, J = 13.0, 1.9 Hz, 1H), |
| | | | | | 7.27 (dd, J = 8.2, 1.9 Hz, 1H), |
| | | | | | 6.87 (dd, J = 9.6, 8.3 Hz, 1H), |
| | | | | | 5.38 (s, 2H), 3.37 (s, 3H). |
| 187 | 187 | 535.63 | N-methyl-2-{[8-(1- | 99.5% | ¹H NMR (400 MHz, |
| | | | methyl-1H-indol- | [M + H]⁺ = 536.2 | DMSO) δ 9.04 (s, 1H), 8.80 (d, |
| | | | 6-yl)quinoxalin-6- | | J = 1.8 Hz, 1H), 8.70 (d, J = 1.8 Hz, |
| | | | yl]amino}-N- | | 1H), 8.64 (s, 2H), 8.32 (s, |
| | | | [(pyrimidin-5- | | 1H), 7.95 (dd, J = 8.0, 1.5 Hz, |
| | | | yl)methyl]benzene- | | 1H), 7.79 (d, J = 2.6 Hz, 1H), |
| | | | 1-sulfonamide | | 7.78 (dd, J = 8.3, 0.9 Hz, 2H), |
| | | | | | 7.74-7.68 (m, 2H), 7.63 (dd, J = 8.2, |
| | | | | | 0.4 Hz, 2H), 7.52 (d, J = 2.5 Hz, |
| | | | | | 1H), 7.41 (d, J = 3.0 Hz, |
| | | | | | 1H), 7.34 (dd, J = 8.2, 1.5 Hz, |
| | | | | | 1H), 7.32-7.28 (m, 1H), |
| | | | | | 6.49 (dd, J = 3.1, 0.6 Hz, 1H), |
| | | | | | 4.37 (s, 2H), 3.83 (s, 3H), 2.74 (s, |
| | | | | | 3H). |
| 188 | 188 | 394.42 | 8-(4- | 97.4% | ¹H NMR (400 MHz, |
| | | | fluorophenyl)-N- | [M + H]⁺ = 393.1 | DMSO) δ 8.98 (s, 1H), 8.83 (d, |
| | | | (4- | | J = 1.8 Hz, 1H), 8.73 (d, J = 1.8 Hz, |
| | | | methanesulfonylpyridin- | | 1H), 8.57 (d, J = 5.1 Hz, |
| | | | 3- | | 1H), 8.46 (s, 1H), 7.85 (dd, J = 5.1, |
| | | | yl)quinoxalin-6- | | 0.6 Hz, 1H), 7.81 (d, J = 2.6 Hz, |
| | | | amine | | 1H), 7.77-7.67 (m, 1H), |
| | | | | | 7.72 (d, J = 3.0 Hz, 1H), |
| | | | | | 7.59 (d, J = 2.5 Hz, 1H), 7.33 (m, |
| | | | | | 2H), 3.37 (s, 3H). |
| 189 | 189 | 443.53 | 8-(1,4-dimethyl- | 95.5% | ¹H NMR (400 MHz, |
| | | | 1H-indol-6-yl)-N- | [M + H]⁺ = 442.2 | DMSO) δ 8.99 (s, 1H), 8.80 (d, |
| | | | (4- | | J = 1.7 Hz, 1H), 8.72 (d, J = 1.8 Hz, |
| | | | methanesulfonylpyridin- | | 1H), 8.56 (d, J = 5.1 Hz, |
| | | | 3- | | 1H), 8.44 (s, 1H), 7.85 (d, J = 5.2 Hz, |
| | | | yl)quinoxalin-6- | | 1H), 7.83 (d, J = 2.6 Hz, |
| | | | amine | | 1H), 7.55-7.52 (m, 2H), |
| | | | | | 7.37 (d, J = 3.2 Hz, 1H), 7.12 (s, 1H), |
| | | | | | 6.50 (d, J = 3.3 Hz, 1H 3.82 (s, |
| | | | | | 3H), 3.39 (s, 3H), 2.54 (s, 3H). |
| 190 | 190 | 447.53 | 8-(2-amino-1,3- | 99.2% | ¹H NMR (400 MHz, DMSO- |
| | | | benzothiazol-5-yl)- | [M + H]⁺ = 448.3 | d6) δ 8.81 (d, J = 1.9 Hz, 1H), |
| | | | N-(2- | | 8.71 (d, J = 1.8 Hz, 1H), 8.37 (s, |
| | | | methanesulfonylphenyl)quinoxalin- | | 1H), 7.95 (dd, J = 7.6, 1.3 Hz, |
| | | | 6-amine | | 1H), 7.79 (d, J = 2.6 Hz, 1H), |
| | | | | | 7.77-7.70 (m, 1H), 7.62 (d, J = 1.6 Hz, |
| | | | | | 1H), 7.55 (d, J = 2.6 Hz, |
| | | | | | 1H), 7.53 (s, 2H), |
| | | | | | 7.36-7.32 (m, 1H), 7.31 (dd, J = 8.2, |
| | | | | | 1.7 Hz, 1H), 3.27 (s, 3H). |
| 191 | 191 | 445.56 | N-(2- | 96.1% | ¹H NMR (400 MHz, |
| | | | methanesulfonylphenyl)- | [M + H]⁺ = 446.3 | DMSO) δ 8.82 (d, J = 1.8 Hz, |
| | | | 8-(3- | | 1H), 8.71 (d, J = 1.8 Hz, 1H), |
| | | | methyl-1- | | 8.39 (s, 1H), 8.07 (d, J = 8.3 Hz, |
| | | | benzothiophen-5- | | 1H), 8.02 (d, J = 1.2 Hz, 1H), |
| | | | yl)quinoxalin-6- | | 7.96 (dd, J = 7.9, 1.4 Hz, 1H), |
| | | | amine | | 7.87 (d, J = 2.6 Hz, 1H), |
| | | | | | 7.77 (dd, J = 8.2, 1.2 Hz, 2H), |
| | | | | | 7.73 (ddd, J = 8.2, 6.8, 1.5 Hz, 1H), |
| | | | | | 7.68 (dd, J = 8.4, 1.6 Hz, 1H), |
| | | | | | 7.57 (d, J = 2.5 Hz, 1H), |
| | | | | | 7.47 (d, J = 1.0 Hz, 1H), 7.35 (ddd, J = 8.2, |
| | | | | | 6.9, 1.5 Hz, 1H), 3.28 (s, |
| | | | | | 3H), 2.45 (d, J = 1.0 Hz, 3H). |
| 192 | 192 | 432.54 | 8-(3,5- | 99.0% | ¹H NMR (400 MHz, |
| | | | diethylphenyl)-N- | [M + H]⁺ = 433.3 | DMSO) δ 8.98 (s, 1H), 8.80 (d, |
| | | | (4- | | J = 1.8 Hz, 1H), 8.71 (d, J = 1.8 Hz, |
| | | | methanesulfonylpyridin- | | 1H), 8.56 (d, J = 4.9 Hz, |
| | | | 3- | | 1H), 8.43 (s, 1H), 7.84 (d, J = 5.1 Hz, |
| | | | yl)quinoxalin-6- | | 1H), 7.77 (d, J = 2.6 Hz, |
| | | | amine | | 1H), 7.53 (d, J = 2.5 Hz, 1H), |
| | | | | | 7.28 (d, J = 1.4 Hz, 2H), 7.13 (s, |
| | | | | | 1H), 3.37 (s, 2H), 2.67 (q, J = 7.5 Hz, |
| | | | | | 4H), 1.23 (t, J = 7.6 Hz, |
| | | | | | 6H). |
| 193 | 193 | 494.62 | 3-{[8-(3-methyl-1- | 98.3% | ¹H NMR (400 MHz, DMSO-d6) |
| | | | benzothiophen-5- | [M + H]⁺ = 495.3 | 9.28 (s, 1H), 8.85 (s, 1H), |
| | | | yl)quinoxalin-6- | | 8.80 (d, J = 1.9 Hz, 1H), |
| | | | yl]amino}-N-[(3S)- | | 8.79 (d, J = 7.1 Hz, 1H), 8.67 (d, J = 1.8 Hz, |
| | | | 1- | | 1H), 8.36 (d, J = 5.0 Hz, |
| | | | methylpyrrolidin- | | 1H), 8.06 (dd, J = 8.3, 0.4 Hz, |
| | | | 3-yl]pyridine-4- | | 1H), 8.01 (d, J = 1.2 Hz, 1H), |
| | | | carboxamide | | 7.70 (d, J = 2.6 Hz, 1H), |
| | | | | | 7.66 (dd, J = 8.3, 1.5 Hz, 1H), |
| | | | | | 7.58 (d, J = 4.9 Hz, 1H), 7.47 (d, J = 1.0 Hz, |
| | | | | | 1H), 7.44 (d, J = 2.6 Hz, |
| | | | | | 1H), 4.26 (dtt, J = 9.4, 6.9, 4.9 Hz, |
| | | | | | 1H), 2.58-2.50 (m, 1H), |
| | | | | | 2.49-2.41 (m, 1H), 2.45 (d, J = 1.0 Hz, |
| | | | | | 3H), 2.30 (td, J = 8.3, |
| | | | | | 6.1 Hz, 1H), 2.22 (dd, J = 9.4, |
| | | | | | 4.8 Hz, 1H), 2.13 (s, 3H), |
| | | | | | 2.02 (dddd, J = 13.2, 9.1, 7.9, 5.7 Hz, |
| | | | | | 1H), 1.61 (ddt, J = 11.3, |
| | | | | | 8.1, 5.6 Hz, 1H). |

| Cpd. | Ex. | | | | |
|---|---|---|---|---|---|
| No. | No. | MW | IUPAC name | LC-MS | ¹H-NMR |
| 194 | 194 | 494.62 | 3-{[8-(3-methyl-1- | 97.4% | ¹H NMR (400 MHz, DMSO-d6) |
| | | | benzothiophen-5- | [M + H]⁺ = 493.4 | δ 9.28 (s, 1H), 8.85 (s, 1H), |
| | | | yl)quinoxalin-6- | | 8.80 (d, J = 1.5 Hz, 1H), 8.78 (s, |
| | | | yl]amino}-N-[(3R)- | | 1H), 8.67 (d, J = 1.2 Hz, 1H), |
| | | | 1- | | 8.36 (d, J = 4.8 Hz, 1H), |
| | | | methylpyrrolidin- | | 8.06 (d, J = 8.3 Hz, 1H), 8.01 (s, 1H), |
| | | | 3-yl]pyridine-4- | | 7.70 (d, J = 2.2 Hz, 1H), |
| | | | carboxamide | | 7.66 (d, J = 7.9 Hz, 1H), 7.58 (d, J = 4.8 Hz, |
| | | | | | 1H), 7.47 (s, 1H), |
| | | | | | 7.44 (d, J = 2.2 Hz, 1H), |
| | | | | | 4.32-4.21 (m, 1H), 2.58-2.53 (m, 1H), |
| | | | | | 2.45 (s, 3H), 2.44-2.41 (m, |
| | | | | | 1H), 2.30 (q, J = 7.8 Hz, 1H), |
| | | | | | 2.21 (dd, J = 9.2, 4.5 Hz, 1H), |
| | | | | | 2.12 (s, 3H), 2.08-1.93 (m, |
| | | | | | 1H), 1.60 (dq, J = 12.9, 7.8, 6.7 Hz, |
| | | | | | 1H). |
| 195 | 195 | 443.53 | 8-(1,5-dimethyl- | 99.6% | ¹H NMR (400 MHz, DMSO-d₆) |
| | | | 1H-indol-6-yl)-N- | [M + H]⁺ = 444.3 | δ 8.99 (s, 1H), 8.79 (d, J = 1.8 Hz, |
| | | | (4- | | 1H), 8.63 (d, J = 1.8 Hz, |
| | | | methanesulfonylpyridin- | | 1H), 8.56 (d, J = 5.1 Hz, 1H), |
| | | | 3- | | 8.47 (s, 1H), 7.84 (dd, J = 5.1, |
| | | | yl)quinoxalin-6- | | 0.5 Hz, 1H), 7.65 (d, J = 2.6 Hz, |
| | | | amine | | 1H), 7.60 (d, J = 2.6 Hz, 1H), |
| | | | | | 7.44 (s, 1H), 7.32 (s, 1H), |
| | | | | | 7.32 (d, J = 3.1 Hz, 1H), 6.40 (dd, J = 3.0, |
| | | | | | 0.8 Hz, 1H), 3.75 (s, 3H), |
| | | | | | 3.38 (s, 3H), 2.04 (s, 3H). |
| 197 | 197 | 495.56 | 3-{[8-(4-fluoro-1- | 96.9% | ¹H NMR (400 MHz, DMSO-d6) |
| | | | methyl-1H-indol- | [M + H]⁺ = 496.3 | δ 9.31 (s, 1H), 8.94 (d, J = 6.8 Hz, |
| | | | 6-yl)quinoxalin-6- | | 1H), 8.86 (s, 1H), 8.80 (d, J = 1.8 Hz, |
| | | | yl]amino}-N-(1- | | 1H), 8.70 (d, J = 1.8 Hz, |
| | | | methylpyrrolidin- | | 1H), 8.36 (d, J = 5.0 Hz, |
| | | | 3-yl)pyridine-4- | | 1H), 7.71 (d, J = 2.6 Hz, 1H), |
| | | | carboxamide | | 7.63 (d, J = 5.0 Hz, 1H), 7.60 (s, |
| | | | | | 1H), 7.47 (d, J = 3.2 Hz, 1H), |
| | | | | | 7.46 (d, J = 2.7 Hz, 1H), |
| | | | | | 7.17 (dd, J = 11.7, 1.0 Hz, 1H), |
| | | | | | 6.56 (dd, J = 3.1, 0.7 Hz, 1H), |
| | | | | | 4.35 (dtd, J = 13.6, 7.1, 3.9 Hz, 1H), |
| | | | | | 3.87 (s, 3H), 2.95-2.87 (m, |
| | | | | | 1H), 2.86-2.74 (m, 1H), |
| | | | | | 2.72-2.58 (m, 2H), 2.39 (s, 3H), |
| | | | | | 2.11 (dq, J = 14.4, 8.1 Hz, 1H), |
| | | | | | 1.75 (dq, J = 12.8, 6.5 Hz, 1H). |
| 198 | 198 | 396.41 | 3-{[8-(1-methyl- | 86.8% | ¹H NMR (400 MHz, DMSO-d₆): |
| | | | 1H-indol-6- | [M + H]⁺ = 396.3 | δ 11.66 (s, 1H), 8.93 (d, J = 1.8 Hz, |
| | | | yl)quinoxalin-6- | | 1H), 8.85 (d, J = 1.8 Hz, |
| | | | yl]oxy}pyridine-4- | | 1H), 8.67 (d, J = 2.4 Hz, 1H), |
| | | | carboxylic acid | | 8.34 (s, 1H), 8.10 (d, J = 2.5 Hz, |
| | | | | | 2H), 7.75 (d, J = 5.2 Hz, 1H), |
| | | | | | 7.73 (s, 1H), 7.66 (d, J = 8.2 Hz, |
| | | | | | 1H), 7.42 (d, J = 3.0 Hz, 1H), |
| | | | | | 7.35 (dd, J = 8.2, 1.5 Hz, 1H), |
| | | | | | 6.51 (dd, J = 3.1, 0.8 Hz, 1H), |
| | | | | | 3.85 (s, 3H). |
| 199 | 199 | 529.68 | 2-{[8-(3-methyl-1- | 95.1 | ¹H NMR (400 MHz, DMSO-d₆) |
| | | | benzothiophen-5- | [M + H]⁺ = 530.3 | δ 8.83 (d, J = 1.8 Hz, 1H), |
| | | | yl)quinoxalin-6- | | 8.72 (d, J = 1.8 Hz, 1H), 8.19 (d, J = 8.2 Hz, |
| | | | yl]amino}-N-(1- | | 1H), 8.08 (s, 1H), |
| | | | methylpyrrolidin- | | 8.08 (dd, J = 8.3, 0.5 Hz, 1H), |
| | | | 3-yl)benzene-1- | | 8.01 (dd, J = 1.6, 0.5 Hz, 1H), |
| | | | sulfonamide | | 7.88 (dd, J = 8.0, 1.5 Hz, 1H), |
| | | | | | 7.80 (d, J = 2.6 Hz, 1H), 7.77 (dd, J = 8.2, |
| | | | | | 0.9 Hz, 1H), 7.68 (dd, J = 8.3, |
| | | | | | 1.6 Hz, 1H), |
| | | | | | 7.66-7.60 (m, 2H), 7.48 (d, J = 1.1 Hz, |
| | | | | | 1H), 7.20 (ddd, J = 8.2, 7.4, 1.1 Hz, |
| | | | | | 1H), 3.71-3.62 (m, 1H), |
| | | | | | 2.45 (d, J = 1.2 Hz, 3H), |
| | | | | | 2.40-2.31 (m, 2H), 2.23-2.13 (m, |
| | | | | | 2H), 2.06 (s, 3H), |
| | | | | | 1.92-1.80 (m, 1H), 1.48-1.39 (m, 1H). |
| 200 | 200 | 489.57 | N-(5-{7-[(4- | 85.7 | ¹H NMR (400 MHz, DMSO-d₆) |
| | | | methanesulfonylpyridin- | [M + H]⁺ = 490.3 | δ 11.42 (s, 1H), 9.01 (s, 1H), |
| | | | 3- | | 8.83 (d, J = 1.8 Hz, 1H), |
| | | | yl)amino]quinoxalin- | | 8.73 (d, J = 1.9 Hz, 1H), 8.57 (d, J = 5.1 Hz, |
| | | | 5-yl}-1- | | 1H), 8.47 (s, 1H), |
| | | | benzothiophen-2- | | 7.93 (t, J = 0.6 Hz, 1H), 7.92 (dt, J = 10.8, |
| | | | yl)acetamide | | 0.6 Hz, 1H), 7.85 (dd, J = 2.5, |
| | | | | | 1.2 Hz, 1H), 7.85 (dd, J = 5.1, |
| | | | | | 0.5 Hz, 1H), 7.58 (d, J = 2.5 Hz, |
| | | | | | 1H), 7.49 (dd, J = 8.2, 1.7 Hz, |
| | | | | | 1H), 6.98 (d, J = 0.7 Hz, |
| | | | | | 1H), 3.38 (s, 3H), 2.15 (s, 3H). |
| 201 | 201 | 476.57 | 8-[2- | 97.8 | ¹H NMR (400 MHz, DMSO-d₆) |
| | | | (dimethylamino)- | [M + H]⁺ = 477.3 | δ 9.01 (s, 1H), 8.83 (d, J = 1.7 Hz, |
| | | | 1,3-benzothiazol- | | 1H), 8.73 (d, J = 1.8 Hz, |
| | | | 5-yl]-N-(4- | | 1H), 8.56 (d, J = 5.1 Hz, 1H), |
| | | | methanesulfonylpyridin- | | 8.45 (s, 1H), 7.89-7.81 (m, |
| | | | 3- | | 3H), 7.74 (d, J = 1.3 Hz, 1H), |
| | | | yl)quinoxalin-6- | | 7.60 (d, J = 2.5 Hz, 1H), |
| | | | amine | | 7.35 (dd, J = 8.2, 1.6 Hz, 1H), |
| | | | | | 3.37 (s, 3H), 3.17 (s, 6H). |
| 202 | 202 | 462.55 | N-(4- | 96.6 | ¹H NMR (400 MHz, DMSO-d₆) |
| | | | methanesulfonylpyridin- | [M + H]⁺ = 463.3 | δ 9.00 (s, 1H), 8.83 (d, J = 1.8 Hz, |
| | | | 3-yl)-8-[2- | | 1H), 8.73 (d, J = 1.8 Hz, |
| | | | (methylamino)- | | 1H), 8.56 (d, J = 5.1 Hz, 1H), |
| | | | 1,3-benzothiazol- | | 8.45 (s, 1H), 7.99 (q, J = 4.5 Hz, |
| | | | 5-yl]quinoxalin-6- | | 1H), 7.85-7.82 (m, 2H), |
| | | | amine | | 7.77 (dd, J = 8.1, 0.6 Hz, 1H), |
| | | | | | 7.70 (dd, J = 1.8, 0.5 Hz, 1H), |
| | | | | | 7.59 (d, J = 2.5 Hz, 1H), 7.32 (dd, J = 8.1, |
| | | | | | 1.7 Hz, 1H), 3.37 (s, 3H), |
| | | | | | 2.96 (d, J = 4.7 Hz, 3H). |
| 203 | 203 | 447.53 | 8-(2-amino-1- | 89.8 | ¹H NMR (400 MHz, DMSO-d₆) |
| | | | benzothiophen-5- | [M + H]⁺ = 448.1 | δ 9.00 (s, 1H), 8.81 (d, J = 1.8 Hz, |
| | | | yl)-N-(4- | | 1H), 8.72 (dd, J = 1.9, 0.7 Hz, |
| | | | methanesulfonylpyridin- | | 1H), 8.57 (dd, J = 5.1, 0.7 Hz, |
| | | | 3- | | 1H), 7.85 (dd, J = 5.1, 0.6 Hz, |
| | | | yl)quinoxalin-6- | | 1H), 7.81 (d, J = 2.6 Hz, |
| | | | amine | | 1H), 7.65 (dt, J = 8.2, 0.7 Hz, |
| | | | | | 1H), 7.56 (m, 2H), 7.22 (ddd, J = 8.1, |
| | | | | | 1.8, 0.5 Hz, 2H), 6.14 (s, |
| | | | | | 2H), 6.08 (s, 1H), 3.38 (s, 3H). |
| 204 | 204 | 431.30 | N-(5- | 95.0 | ¹H NMR (400 MHz, DMSO-d₆) |
| | | | bromopyrimidin- | [M + H]⁺ = 433.0 | δ 9.38 (s, 1H), 8.90 (d, J = 1.8 Hz, |
| | | | 4-yl)-8-(1-methyl- | | 1H), 8.84 (d, J = 1.8 Hz, |
| | | | 1H-indol-6- | | 1H), 8.70 (s, 2H), 8.58 (d, J = 2.4 Hz, |
| | | | yl)quinoxalin-6- | | 1H), 8.29 (d, J = 2.5 Hz, |
| | | | amine | | 1H), 7.74 (s, 1H), 7.64 (d, J = 8.2 Hz, |
| | | | | | 1H), 7.41 (d, J = 3.1 Hz, |
| | | | | | 1H), 7.38 (dd, J = 8.2, 1.4 Hz, |
| | | | | | 1H), 6.49 (dd, J = 3.0, 0.7 Hz, |
| | | | | | 1H), 3.84 (s, 3H). |
| 205 | 205 | 411.48 | 3-{[8-(3-methyl-1- | 96.0 | ¹H NMR (400 MHz, DMSO-d₆) |
| | | | benzothiophen-5- | [M + H]⁺ = 412.2 | δ 9.63 (s, 1H), 8.94 (s, 1H), |
| | | | yl)quinoxalin-6- | | 8.82 (d, J = 1.9 Hz, 1H), |
| | | | yl]amino}pyridine- | | 8.69 (d, J = 1.9 Hz, 1H), 8.32 (d, J = 4.9 Hz, |
| | | | 4-carboxamide | | 2H), 8.06 (d, J = 8.4 Hz, |
| | | | | | 1H), 8.02 (d, J = 1.6 Hz, 1H), |
| | | | | | 7.86 (s, 1H), 7.77 (d, J = 2.6 Hz, |
| | | | | | 1H), 7.68 (dd, J = 8.3, 1.7 Hz, |
| | | | | | 1H), 7.64 (d, J = 5.0 Hz, 1H), |
| | | | | | 7.59 (d, J = 2.6 Hz, 1H), |
| | | | | | 7.47 (d, J = 1.4 Hz, 1H), 2.45 (d, J = 1.2 Hz, |
| | | | | | 3H). |
| 206 | 206 | 508.60 | N-(1- | 93.8 | ¹H NMR (400 MHz, DMSO-d₆) |
| | | | acetylazetidin-3- | [M + H]⁺ = 509.7 | δ 9.30 (d, J = 7.4 Hz, 2H), |
| | | | yl)-3-{[8-(3- | | 8.88 (s, 1H), 8.80 (d, J = 1.9 Hz, 1H), |
| | | | methyl-1- | | 8.68 (d, J = 1.9 Hz, 1H), |
| | | | benzothiophen-5- | | 8.38 (d, J = 5.0 Hz, 1H), 8.06 (dd, J = 8.3, |
| | | | yl)quinoxalin-6- | | 0.6 Hz, 1H), 8.00 (dd, J = 1.6, |
| | | | yl]amino}pyridine- | | 0.7 Hz, 1H), 7.71 (d, J = 2.6 Hz, |
| | | | 4-carboxamide | | 1H), 7.66 (dd, J = 8.3, 1.7 Hz, |
| | | | | | 1H), 7.62 (dd, J = 5.0, 0.6 Hz, |
| | | | | | 1H), 7.47 (m, 2H), 4.26 (t, J = 8.3 Hz, |
| | | | | | 1H), 4.10 (q, J = 5.2 Hz, |
| | | | | | 1H), 3.99 (t, J = 8.9 Hz, 1H), |
| | | | | | 3.83 (dd, J = 8.6, 5.4 Hz, 1H), |
| | | | | | 3.72 (dd, J = 9.9, 5.3 Hz, 1H), |
| | | | | | 2.46 (s, 1.5H), 2.45 (s, 1.5H), |
| | | | | | 1.65 (s, 3H). |
| 207 | 207 | 471.52 | 3-{[8-(1-methyl- | 89.9% | ¹H NMR (400 MHz, DMSO-d₆): |
| | | | 1H-indol-6- | [M + H]⁺ = 472.1 | δ 10.80 (s, 1H), 9.06 (s, 1H), |
| | | | yl)quinoxalin-6- | | 8.90 (s, 1H), 8.75 (d, J = 2.0 Hz, |
| | | | yl]amino}-N- | | 2H), 8.64 (d, J = 1.9 Hz, 1H), |
| | | | (pyridin-3- | | 8.43 (d, J = 5.0 Hz, 1H), |
| | | | yl)pyridine-4- | | 8.27 (dd, J = 4.7, 1.5 Hz, 1H), |
| | | | carboxamide | | 7.99 (d, J = 8.5 Hz, 1H), |
| | | | | | 7.70-7.67 (m, 2H), 7.65-7.64 (m, 1H), |
| | | | | | 7.61 (dd, J = 8.2, 0.7 Hz, 1H), |
| | | | | | 7.46 (d, J = 2.6 Hz, 1H), |
| | | | | | 7.40 (d, J = 3.0 Hz, 1H), 7.31 (dd, J = 8.3, |
| | | | | | 4.7 Hz, 1H), 7.27 (dd, J = 8.2, |
| | | | | | 1.5 Hz, 1H), 6.48 (dd, J = 3.1, |
| | | | | | 0.9 Hz, 1H), 3.82 (s, 3H). |
| 208 | 208 | 526.66 | 2-{[8-(1-methyl- | 97.7% | ¹H NMR (400 MHz, DMSO-d₆): |
| | | | 1H-indol-6- | [M + H]⁺ = 527.3 | δ 8.81 (d, J = 1.9 Hz, 1H), |
| | | | yl)quinoxalin-6- | | 8.72 (d, J = 1.8 Hz, 1H), 8.03 (s, 1H), |
| | | | yl]amino}-N-(1- | | 8.00 (s, 1H)7.90 (dd, J = 8.0, |
| | | | methylpiperidin-3- | | 1.6 Hz, 1H), 7.81-7.69 (m, |
| | | | yl)benzene-1- | | 3H), 7.66-7.57 (m, 3H), |
| | | | sulfonamide | | 7.41 (d, J = 3.1 Hz, 1H), 7.35 (dd, J = 8.2, |
| | | | | | 1.5 Hz, 1H), 7.19 (ddd, J = 8.2, |
| | | | | | 7.3, 1.1 Hz, 1H), 6.49 (dd, |
| | | | | | J = 3.1, 0.9 Hz, 1H), 3.84 (s, |
| | | | | | 3H), 3.17-3.06 (m, 1H), |
| | | | | | 2.47-2.41 (m, 1H), 2.38-2.29 (m, |
| | | | | | 1H), 1.94 (s, 3H), |
| | | | | | 1.78-1.63 (m, 2H), 1.48 (d, J = 11.0 Hz, |
| | | | | | 2H), 1.27-1.13 (m, 1H), |
| | | | | | 1.08-0.96 (m, 1H). |
| 209 | 209 | 513.62 | 2-{[8-(1-methyl- | 86.6% | ¹H NMR (400 MHz, DMSO-d⁶) |
| | | | 1H-indol-6- | [M + H]⁺ = 514.3 | δ 8.81 (d, J = 1.9 Hz, 1H), |
| | | | yl)quinoxalin-6- | | 8.71 (d, J = 1.8 Hz, 1H), 8.10 (d, J = 6.1 Hz, |
| | | | yl]amino}-N- | | 1H), 8.03 (s, 1H), |
| | | | (oxan-3- | | 7.90 (dd, J = 8.0, 1.5 Hz, 1H), |
| | | | yl)benzene-1- | | 7.76-7.70 (m, 3H), 7.65-7.58 (m, |
| | | | sulfonamide | | 3H), 7.40 (d, J = 3.1 Hz, 1H), |
| | | | | | 7.35 (dd, J = 8.2, 1.5 Hz, 1H), |
| | | | | | 7.21-7.16 (m, 1H), 6.49 (dd, J = 3.1, |
| | | | | | 0.8 Hz, 1H), 3.83 (s, 3H), |
| | | | | | 3.55-3.49 (m, 2H), |
| | | | | | 3.20-3.13 (m, 1H), 3.08 (s, 1H), |
| | | | | | 1.68-1.45 (m, 2H), 1.31-1.21 (m, |
| | | | | | 3H). |
| 210 | 210 | 422.52 | N-(4- | 98.5% | ¹H NMR (400 MHz, DMSO-d₆): |
| | | | methanesulfonylpyridin- | [M + H]⁺ = 423.3 | δ 9.00 (s, 1H), 8.83 (d, J = 1.8 Hz, |
| | | | 3-yl)-8-[3- | | 1H), 8.73 (d, J = 1.8 Hz, |
| | | | (methylsulfanyl)phenyl]quinoxalin- | | 1H), 8.57 (d, J = 5.1 Hz, 1H), |
| | | | 6-amine | | 8.46 (s, 1H), 7.85 (dd, J = 5.1, |
| | | | | | 0.6 Hz, 1H), 7.83 (d, J = 2.6 Hz, |
| | | | | | 1H), 7.60 (d, J = 2.6 Hz, 1H), |
| | | | | | 7.53-7.52 (m, 1H), |
| | | | | | 7.47-7.42 (m, 2H), 7.36-7.33 (m, |
| | | | | | 1H), 3.38 (s, 3H), 2.52 (s, 3H). |
| 211 | 211 | 473.32 | 8-(4-bromo-3- | 97.4% | ¹H NMR (400 MHz, DMSO-d₆): |
| | | | fluorophenyl)-N- | [M − H]⁻ = 471.7 | δ 8.98 (s, 1H), 8.85 (d, J = 1.9 Hz, |
| | | | (4- | | 1H), 8.75 (d, J = 1.9 Hz, |
| | | | methanesulfonylpyridin- | | 1H), 8.58 (d, J = 5.1 Hz, 1H), |
| | | | 3- | | 8.48 (s, 1H), 7.88 (d, J = 2.6 Hz, |
| | | | yl)quinoxalin-6- | | 1H), 7.87-7.82 (m, 2H), |
| | | | amine | | 7.71 (dd, J = 10.2, 2.0 Hz, 1H), |
| | | | | | 7.63 (d, J = 2.5 Hz, 1H), 7.48 (dd, J = 8.1, |
| | | | | | 2.0 Hz, 1H), 3.37 (s, 3H). |
| 212 | 212 | 469.36 | 8-(4-bromo-2- | 87.8% | ¹H NMR (400 MHz, DMSO-d₆): |
| | | | methylphenyl)-N- | [M − H]⁻ = 467.6 | δ 8.95 (s, 1H), 8.81 (d, J = 1.9 Hz, |
| | | | (4- | | 1H), 8.64 (d, J = 1.9 Hz, |
| | | | methanesulfonylpyridin- | | 1H), 8.56 (d, J = 5.1 Hz, 1H), |
| | | | 3- | | 8.47 (s, 1H), 7.83 (d, 1H), |
| | | | yl)quinoxalin-6- | | 7.62 (q, J = 2.6 Hz, 2H), 7.56 (dd, |
| | | | amine | | 1H), 7.47 (ddd, J = 8.1, 2.2, 0.6 Hz, |
| | | | | | 1H), 7.22 (d, J = 8.2 Hz, |
| | | | | | 1H), 3.35 (s, 3H), 2.00 (s, 3H). |
| 213 | 213 | 502.48 | N-(4- | 98.9% | ¹H NMR (400 MHz, DMSO-d₆) |
| | | | methanesulfonylpyridin- | [M + H]⁺ = 503.0 | δ 9.00 (s, 1H), 8.87 (d, J = 1.9 Hz, |
| | | | 3-yl)-8-[4- | | 1H), 8.74 (d, J = 1.8 Hz, |
| | | | (pentafluoro-λ⁶- | | 1H), 8.59 (d, J = 5.1 Hz, 1H), |
| | | | sulfanyl)phenyl]quinoxalin- | | 8.52 (s, 1H), 8.07-8.02 (m, |
| | | | 6-amine | | 2H), 7.92-7.87 (m, 3H), |
| | | | | | 7.86 (d, J = 5.1 Hz, 1H), 7.65 (d, J = 2.6 Hz, |
| | | | | | 1H), 3.37 (s, 3H). |
| 214 | 214 | 496.59 | 3-{[8-(2-amino- | 99.4% | ¹H NMR (400 MHz, DMSO-d₆): |
| | | | 1,3-benzothiazol- | [M + H]⁺ = 497.4 | δ 9.21 (s, 1H), 8.82 (s, 1H), |
| | | | 5-yl)quinoxalin-6- | | 8.79 (d, 1H), 8.77 (d, J = 1.7 Hz, |
| | | | yl]amino}-N-(1- | | 1H), 8.65 (d, J = 1.8 Hz, |
| | | | methylpyrrolidin- | | 1H), 8.36 (d, J = 4.9 Hz, 1H), |
| | | | 3-yl)pyridine-4- | | 7.75 (d, J = 8.1 Hz, 1H), |
| | | | carboxamide | | 7.62 (d, J = 2.6 Hz, 1H), 7.59 (d, J = 1.4 Hz, |
| | | | | | 1H), 7.57 (d, J = 4.9 Hz, |
| | | | | | 1H), 7.53 (s, 2H), 7.39 (d, J = 2.5 Hz, |
| | | | | | 1H), 7.28 (dd, J = 8.1, |
| | | | | | 1.6 Hz, 1H), 4.31-4.21 (m, |
| | | | | | 1H), 2.62-2.58 (m, 1H), |
| | | | | | 2.39-2.35 (m, 1H), 2.30-2.27 (m, |
| | | | | | 1H), 2.17 (s, 3H), |
| | | | | | 2.05-1.97 (m, 1H), 1.68-1.45 (m, 2H). |
| 215 | 215 | 503.40 | 3-{[8-(4- | 98.3% | ¹H NMR (400 MHz, DMSO-d₆) |
| | | | bromophenyl)quinoxalin- | [M − H]⁻ = 502.0 | δ 9.25 (s, 1H), 8.84-8.75 (m, |
| | | | 6- | | 3H), 8.67 (d, J = 1.8 Hz, 1H), |
| | | | yl]amino}-N-(1- | | 8.36 (d, J = 5.0 Hz, 1H), |
| | | | methylpyrrolidin- | | 7.73-7.68 (m, 2H), 7.64-7.60 (m, |
| | | | 3-yl)pyridine-4- | | 3H), 7.57 (d, J = 5.0 Hz, 1H), |
| | | | carboxamide | | 7.44 (d, J = 2.6 Hz, 1H), |
| | | | | | 4.29-4.19 (m, 1H), 2.49-2.40 (m, |
| | | | | | 1H), 2.32-2.16 (m, 2H), |
| | | | | | 2.13 (s, 3H), 2.05-1.53 (m, 3H). |

### Biological Activity

Biological activity of the compounds of the present invention is determined utilizing the assays described herein below.

### PFKFB3 IC50 Determination Assay

In vitro kinase assay used to determine IC₅₀ values for tested inhibitors is based on a modified ADP-Glo™ system (Promega) and consists of two parts:

1. Kinase reaction—performed in optimized conditions. At this step PFKFB3 phosphorylates its substrate Fructose-6-phosphate using ATP as a source of phosphate to produce Fructose-2,6-bisphosphate and ADP. Reaction is performed at Km values for ATP and substrate using optimized buffer composition and time of the reaction. Human recombinant His-tagged PFKFB3 (PFKFB3 BATCH II SEC) with confirmed activity is produced and purified in-house.
2. Detection of ADP as a product of the reaction using ADP-Glo™ system. This part is conducted by using the commercially available kit ADP-Glo™ Kinase Assay (Promega, cat. No# V9103) according to manufacturer's instruction, modified by 5x dilutions of assay reagents (both ADP-Glo™ Reagent and Kinase Detection Solution). Reproducibility and reliability of this modification is confirmed in an optimization process.

Test compounds are dissolved in DMSO and then transferred to the V-bottom 96-well plate. For IC50 determination ten 10x serial dilutions starting from 100 μM are prepared.

Two mixes are prepared on ice: Mix 1—containing appropriate kinase amount in 2x reaction buffer (100 mM TRIS pH 8.0) and Mix 2—containing 2.31× concentrated substrate (Fructose-6-phosphate) and ATP in MilliQ water. 15 μL per well of Mix 1 is transferred to assay wells of 96-well white plate. Next, 2 μl of 15× concentrated test compound in DMSO is added to Mix 1 for 20 min pre-incubation, followed by addition of Mix 2 (13 μl/well). Total reaction volume is 30 μL per well. Samples are tested in duplicates. Final concentration of DMSO in the reaction is 6.7%. Conditions needed for performing PFKFB3 (PFKFB3 BATCH II SEC) in vitro kinase assay are given below:

| | Final concentration/ |
|---|---|
| Reagent/condition | final condition |
| Buffer | 100 mM Tris, pH 8.0 |
| MgCl₂ | 5 mM |
| KF | 20 mM |
| DTT | 1 mM |
| KH₂PO₄ | 5 mM |
| BSA | 0.02% |
| Tween-20 | 0.005% |
| ATP (Km) | 20 μM |
| (ultrapure, from | |
| ADP-Glo ™ kit) | |
| Substrate Fructose- | 2 μM |
| 6-phosphate (Km) | |
| Sigma cat no. | |
| F3627 | |
| In-house produced | 25 nM |
| human recombinant | |
| PFKFB3 (PFKFB3 | |
| BATCH II SEC) | |
| Time of reaction | 2 h |
| Temperature of | rt |
| reaction | |

This protocol is based on Technical Bulletin, ADP-Glo™ Kinase Assay (Promega) and is adapted to 96-well plate containing 30 μL reaction mixture:

30 μL of 5x diluted ADP-Glo™ Reagent is added to each well of 96-well plate containing 30 μL of reaction mixture. The plate is incubated for 90 minutes on a shaker at RT. 60 μL of 5x diluted Kinase Detection Solution is added to each well of 96-well plate containing 60 μL of the solution (ratio of kinase reaction volume to ADP-Glo™ Reagent volume to Kinase Detection Solution volume is maintained at 1:1:2). Plate is incubated for 40 minutes on a shaker at RT, protected from light. Luminescence is measured in the plate reader Synergy 2 (BioTek).

Luminescent readouts for compounds tested in 10 concentrations (routinely from 100 μM to 1 nM, 10-fold serial dilutions) in duplicates, as well as for positive control, are first normalized to no-substrate negative control by its subtraction. In the next step, % of normalized positive control is calculated for each data point and plotted against test compound concentration:

$$\% of control = 100 \% \times \frac{( {Lum}_{cpd} - {Lum}_{neg} )}{( {Lum}_{pos} - {Lum}_{neg} )}$$

% of control percent of positive control normalized to no-substrate negative control
Lum_{cpd} luminescence of test compound
Lum_{neg} luminescence of no-substrate negative control
Lumₚₒₛ luminescence of positive control

IC₅₀ parameter is determined by the GraphPad Prism 5.0 software [log(inhibitor) vs. response −Variable slope (four parameters)].

IC₅₀ values of compounds of the present invention are shown in Table 2 below.

Compounds are classified according to their IC₅₀ values in the assays described above in three groups:

Group A IC₅₀ is in the range of ≥1 nM to <1 μM
Group B IC₅₀ is in the range of ≥1 μM to <10 μM
Group C IC₅₀ is in the range of ≥10 μM to 100 μM

| Compound | Example | |
|---|---|---|
| No. | No. | PFKFB3 IC50 |
| 1 | 1 | B |
| 2 | 2 | A |
| 3 | 3 | A |
| 4 | 4 | A |
| 5 | 5 | B |
| 6 | 6 | A |
| 7 | 7 | A |
| 8 | 8 | A |
| 9 | 9 | A |
| 10 | 10 | A |
| 11 | 11 | A |
| 13 | 13 | A |
| 14 | 14 | A |
| 15 | 15 | B |
| 16 | 16 | A |
| 17 | 17 | B |
| 18 | 18 | A |
| 19 | 19 | A |
| 20 | 20 | A |
| 21 | 21 | A |
| 22 | 22 | A |
| 23 | 23 | A |
| 24 | 24 | A |
| 25 | 25 | A |
| 26 | 26 | A |
| 27 | 27 | B |
| 28 | 28 | C |
| 29 | 29 | B |
| 30 | 30 | C |
| 31 | 31 | C |
| 32 | 32 | B |
| 33 | 33 | C |
| 34 | 34 | C |
| 35 | 35 | A |
| 36 | 36 | A |
| 37 | 37 | A |
| 38 | 38 | C |
| 39 | 39 | A |
| 40 | 40 | A |
| 41 | 41 | B |
| 42 | 42 | A |
| 43 | 43 | A |
| 44 | 44 | A |
| 45 | 45 | B |
| 46 | 46 | B |
| 47 | 47 | A |
| 48 | 48 | C |
| 49 | 49 | B |
| 50 | 50 | A |
| 51 | 51 | B |
| 52 | 52 | A |
| 53 | 53 | A |
| 54 | 54 | A |
| 55 | 55 | A |
| 56 | 56 | A |
| 57 | 57 | A |
| 58 | 58 | C |
| 59 | 59 | A |
| 60 | 60 | A |
| 61 | 61 | C |
| 62 | 62 | A |
| 63 | 63 | B |
| 65 | 65 | B |
| 66 | 66 | B |
| 67 | 67 | B |
| 68 | 68 | A |
| 69 | 69 | A |
| 71 | 71 | A |
| 72 | 72 | A |
| 73 | 73 | A |
| 74 | 74 | B |
| 75 | 75 | A |
| 81 | 81 | A |
| 82 | 82 | A |
| 83 | 83 | A |
| 84 | 84 | A |
| 85 | 85 | A |
| 88 | 88 | A |
| 89 | 89 | A |
| 90 | 90 | B |
| 91 | 91 | B |
| 92 | 92 | A |
| 93 | 93 | B |
| 94 | 94 | A |
| 95 | 95 | B |
| 96 | 96 | A |
| 97 | 97 | A |
| 98 | 98 | A |
| 99 | 99 | A |
| 100 | 100 | B |
| 101 | 101 | A |
| 102 | 102 | A |
| 103 | 103 | B |
| 104 | 104 | B |
| 110 | 110 | A |
| 115 | 115 | B |
| 116 | 116 | A |
| 117 | 117 | A |
| 118 | 118 | B |
| 119 | 119 | A |
| 120 | 120 | B |
| 121 | 121 | A |
| 122 | 122 | A |
| 123 | 123 | A |
| 124 | 124 | A |
| 125 | 125 | B |
| 126 | 126 | B |
| 127 | 127 | A |
| 128 | 128 | C |
| 129 | 129 | C |
| 130 | 130 | C |
| 131 | 131 | B |
| 132 | 132 | B |
| 135 | 135 | A |
| 136 | 136 | A |
| 137 | 137 | A |
| 140 | 140 | A |
| 141 | 141 | A |
| 142 | 142 | A |
| 143 | 143 | A |
| 144 | 144 | A |
| 145 | 145 | B |
| 146 | 146 | C |
| 147 | 147 | A |
| 148 | 148 | A |
| 149 | 149 | A |
| 150 | 150 | A |
| 151 | 151 | A |
| 152 | 152 | B |
| 153 | 153 | B |
| 154 | 154 | A |
| 157 | 157 | A |
| 158 | 158 | B |
| 160 | 160 | A |
| 161 | 161 | B |
| 162 | 162 | A |
| 163 | 163 | B |
| 164 | 164 | C |
| 165 | 165 | C |
| 166 | 166 | A |
| 167 | 167 | C |
| 168 | 168 | A |
| 169 | 169 | C |
| 170 | 170 | C |
| 171 | 171 | A |
| 172 | 172 | A |
| 173 | 173 | A |
| 174 | 174 | A |
| 175 | 175 | A |
| 176 | 176 | A |
| 178 | 178 | A |
| 179 | 179 | A |
| 181 | 181 | A |
| 182 | 182 | A |
| 183 | 183 | A |
| 184 | 184 | B |
| 185 | 185 | B |
| 186 | 186 | A |
| 187 | 187 | A |
| 188 | 188 | A |
| 189 | 189 | A |
| 190 | 190 | A |
| 191 | 191 | A |
| 192 | 192 | B |
| 193 | 193 | A |
| 194 | 194 | A |
| 195 | 195 | A |
| 198 | 198 | C |
| 199 | 199 | A |
| 200 | 200 | B |
| 201 | 201 | C |
| 202 | 202 | B |
| 203 | 203 | A |
| 204 | 204 | C |
| 205 | 205 | A |
| 206 | 206 | A |
| 207 | 207 | A |
| 208 | 208 | A |
| 209 | 209 | A |
| 210 | 210 | A |
| 211 | 211 | A |
| 212 | 212 | A |
| 213 | 213 | C |
| 214 | 214 | A |
| 215 | 215 | A |

1. Compound A compound of formula (I) 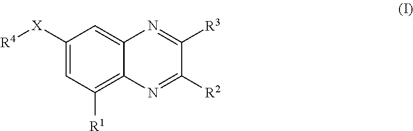
wherein
X denotes N—R⁵ or 0;
R¹ denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, or CA^{X};
R² and R³ denote independently from each other H, —OH, —SH, straight-chain or branched —C₁₋₆-alkyl, straight-chain or branched —C₂₋₆-alkenyl, straight-chain or branched —O—C₁₋₆-alkyl, straight-chain or branched —S—C₁₋₆-alkyl, HaI, —CN, —NH₂, —NH(C₁₋₄-alkyl), or —N(C₁₋₄-alkyl)₂, wherein C₁₋₄-alkyl substituents may be the same or different and may be straight-chain or branched;
R⁴ denotes Ar^{W} or Hetar^{W}, wherein Ar^{W} or Hetar^{W} bears in its ortho-position, relative to the attachment of R⁴ to X, one substituent R^{W1} and may or may not bear further substituents;
R⁵ denotes H, Ar^{X}, Hetar^{X}, Hetcyc^{X}, LA^{X}, or CA^{X};
Ar^{W} denotes a mono-, bi- or tricyclic aromatic ring system with 5, 6, 7, 8, 9, 10, 11, 12, 13, or 14 ring carbon atoms, wherein the ring system may bear, besides the ortho-substituent R^{W1}, no further substituent, one further substituent R^{W2}, or two further substituents R^{W2} and R^{W3}, that may be the same or different;
Ar^{X} denotes a mono-, bi- or tricyclic aromatic ring system with 5, 6, 7, 8, 9, 10, 11, 12, 13, or 14 ring carbon atoms, wherein the ring system may be unsubstituted or mono-, di- or trisubstituted with independently from each other R^{X1}, R^{X2}, or R^{X3};
Ar^{Y} denotes a mono-, bi- or tricyclic aromatic ring system with 5, 6, 7, 8, 9, 10, 11, 12, 13, or 14 ring carbon atoms, wherein the ring system may be unsubstituted or mono-, di- or trisubstituted with independently from each other R^{Y1}, R^{Y2}, or R^{Y3};
Hetar^{W} denotes a mono-, bi- or tricyclic aromatic ring system with 5, 6, 7, 8, 9, 10, 11, 12, 13, or 14 ring atoms wherein 1, 2, 3, 4, or 5 of said ring atoms is/are a hetero atom(s) selected from N, O and/or S and the remaining are carbon atoms, wherein that ring system may bear, besides the ortho-substituent R^{W1}, no further substituent, one further substituent R^{W2}, or two further substituents R^{W2} or R^{W3}, that may be the same or different;
Hetar^{X} denotes a mono-, bi- or tricyclic aromatic ring system with 5, 6, 7, 8, 9, 10, 11, 12, 13, or 14 ring atoms wherein 1, 2, 3, 4, or 5 of said ring atoms is/are a hetero atom(s) selected from N, O and/or S and the remaining are carbon atoms, wherein that aromatic ring system may be unsubstituted or mono-, di- or tri-substituted with independently from each other R^{X1}, R^{X2}, or R^{X3};
Hetar^{Y} denotes a mono-, bi- or tricyclic aromatic ring system with 5, 6, 7, 8, 9, 10, 11, 12, 13, or 14 ring atoms wherein 1, 2, 3, 4, or 5 of said ring atoms is/are a hetero atom(s) selected from N, O and/or S and the remaining are carbon atoms, wherein that aromatic ring system may be unsubstituted or mono-, di- or tri-substituted with independently from each other R^{Y1}, R^{Y2}, or R^{Y3};
Hetcyc^{X} denotes a saturated or partially unsaturated mono-, bi- or tricyclic heterocycle with 3, 4, 5, 6, 7, 8, 9, 10, 11, 12, 13, or 14 ring atoms wherein 1, 2, 3, 4, or 5 ring atom(s) is/are heteroatom(s) selected from N, O and/or S and the remaining ring atoms are carbon atoms, wherein that heterocycle may be unsubstituted or mono-, di- or trisubstituted with R^{X4}, R^{X5}, or R^{X6};
Hetcyc^{Y} denotes a saturated or partially unsaturated mono-, bi- or tricyclic heterocycle with 3, 4, 5, 6, 7, 8, 9, 10, 11, 12, 13, or 14 ring atoms wherein 1, 2, 3, 4, or 5 ring atom(s) is/are heteroatom(s) selected from N, O and/or S and the remaining ring atoms are carbon atoms, wherein that heterocycle may be unsubstituted or mono-, di- or trisubstituted with R^{Y4}, R^{Y5}, or R^{Y6};
R^{W1} denotes HaI, LA^{X}, CA^{X}, Ar^{X}, Ar^{X}Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, —CN, —NO₂, —SO₂NH₂, —SO₂NHR^{W4}, —SO₂NR^{W4}R^{W5}, —NH—SO₂—R^{W6}, —NR^{W4}—SO₂—R^{W6}, —S—R^{W6}, —S(═O)—R^{W6}, —SO₂—R^{W6}, —NH₂, —NHR^{W4}, —NR^{W4}R^{W5}, —OH, —O—R^{W6}, —CHO, —C(═O)—R^{W6}, —COOH, —C(═O)—O—R^{W6}, —C(═O)—NH₂, —C(═O)—NHR^{W4}, —C(═O)—NR^{W4}R^{W5}, —NH—C(═O)—R^{W6}, —NR^{W4}—C(═O)—R^{W6}, —NH—(C₁₋₃-alkylene)-C(═O)—NH₂, —NH—(C₁₋₃-alkylene)-C(═O)—NHR^{W4}, or —NH—(C₁₋₃-alkylene)-C(═O)—NR^{W4}R^{W5},
or
R^{W1} and R⁵ form together a divalent alkylene chain with 1, 2, 3, 4, or 5 chain carbon atoms wherein 2 adjacent CH₂ groups may together be replaced by a —CH═CH— moiety, wherein the divalent alkylene chain may be straight-chain or branched and may be unsubstituted or mono- or di-substituted with independently from each other straight-chain or branched —C₁₋₆-alkyl or ═O (oxo);
R^{W2} and R^{W3} denote independently from each other H, HaI, LA^{X}, CA^{X}, Ar^{X}, Ar^{X}_Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, —CN, —NO₂, —SO₂NH₂, —SO₂NHR^{W4}, —SO₂NR^{W4}R^{W5}, —NH—SO₂—R^{W6}, —NR^{W4}—SO₂—R^{W6}, —S—R^{W6}, —S(═O)—R^{W6}, —SO₂—R^{W6}, —NH₂, —NHR^{W4}, —NR^{W4}R^{W5}—NH—C(═O)—R^{W6}, —NR^{W4}—C(═O)—R^{W6}, —OH, —O—R^{W6}, —CHO, —C(═O)—R^{W6}, —COOH, —C(═O)—O—R^{W6}, —C(═O)—NH₂, —C(═O)—NHR^{W4}, —C(═O)—NR^{W4}R^{W5}, —C(═O)—NH—NH₂, —C(═O)—NH—NHR^{W4}, —NH—(C₁₋₃-alkylene)-C(═O)—NH₂, —NH—(C₁₋₃-alkylene)-C(═O)—NHR^{W4}, or —NH—(C₁₋₃-alkylene)-C(═O)—NR^{W4}R^{W5},
or
two of R^{W1}, R^{W2}, and R^{W3} form a divalent alkylene chain with 3, 4, or 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), or —O—, wherein the C₁₋₆-alkyl and C₁₋₄-alkyl radicals may be straight-chain or branched, and wherein 2 adjacent CH₂ groups may together be replaced by a —CH═CH— moiety, which divalent alkylene chain may be unsubstituted or mono- or di-substituted with independently from each other straight-chain or branched —C₁₋₆-alkyl or ═O (oxo);
R^{X1}, R^{X2}, and R^{X3} denote independently from each other H, HaI, LA^{X}, CA^{X}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, OH, O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, or —NH—(C₁₋₃-alkylene)-C(═O)—NR^{X7}R^{X8}
or
two of R^{X1}, R^{X2}, and R^{X3} form a divalent alkylene chain with 3, 4, or 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), or —O—, wherein the C₁₋₆-alkyl and C₁₋₄-alkyl radicals may be straight-chain or branched and wherein 2 adjacent CH₂ groups may together be replaced by a —CH═CH— moiety, which divalent alkylene chain may be unsubstituted or mono- or di-substituted with independently from each other straight-chain or branched —C₁₋₆-alkyl or ═O (oxo);
R^{X4}, R^{X5}, and R^{X6} denote independently from each other H, HaI, LA^{X}, CA^{X}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, or oxo (═O);
R^{Y1}, R^{Y2}, and R^{Y3} denote independently from each other H, HaI, LA^{Y}, CA^{Y}, —CN, —NO₂, —SF₅, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, —S(═O)—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, or —NH—(C₁₋₃-alkylene)-C(═O)—NR^{Y7}R^{Y8}
or
two of R^{Y1}, R^{Y2}, and R^{Y3} form a divalent alkylene chain with 3, 4, or 5 chain carbon atoms wherein 1 or 2 non-adjacent CH₂ groups of the divalent alkylene chain may be replaced independently from each other by —N(H)—, —N(C₁₋₆-alkyl)-, —N(—C(═O)—C₁₋₄-alkyl), or —O—, wherein the C₁₋₆-alkyl and C₁₋₄-alkyl radicals may be straight-chain or branched, and wherein 2 adjacent CH₂ groups may together be replaced by a —CH═CH— moiety, which divalent alkylene chain may be unsubstituted or mono- or di-substituted with independently from each other straight-chain or branched
—C₁₋₆-alkyl or ═O (oxo);
R^{Y4}, R^{Y5}, and R^{Y6} denote independently from each other H, HaI, LA^{Y}, CA^{Y}, —CN, —NO₂, —SF₅, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, —S(═O)—R^{Y9}, —SO₂—R^{Y9}, —NH₂, —NHR^{Y7}, —NR^{Y7}R^{Y8}, OH, O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, or oxo (═O);
LA^{X} denotes straight-chain or branched C₁₋₆-alkyl which may be unsubstituted or mono-, di- or trisubstituted with independently from each other HaI, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, —NH—C(═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, or oxo (═O), wherein 1 or 2 non-adjacent CH₂ groups of the C₁₋₆-alkyl radical may independently from each other be replaced by O, S, N(H) or N—R^{X7} and/or 1 or 2 non-adjacent CH groups of the C₁₋₆-alkyl radical may independently from each other be replaced by N;
LA^{Y} denotes straight-chain or branched C₁₋₆-alkyl which may be unsubstituted or mono-, di- or trisubstituted with independently from each other HaI, —CN, —NO₂, —SF₅, —SO₂NH₂, —SO₂NHR^{Y7}, —SO₂NR^{Y7}R^{Y8}, —NH—SO₂—R^{Y9}, —NR^{Y7}—SO₂—R^{Y9}, —S—R^{Y9}, —S(═O)—R^{Y9}, —SO₂—R^{Y9}, —NH₂, —NHR^{Y7}, —NR^{Y7}R^{Y8}, —OH, —O—R^{Y9}, —CHO, —C(═O)—R^{Y9}, —COOH, —C(═O)—O—R^{Y9}, —C(═O)—NH₂, —C(═O)—NHR^{Y7}, —C(═O)—NR^{Y7}R^{Y8}, —NH—C(═O)—R^{Y9}, —NR^{Y7}—C(═O)—R^{Y9}, —NH—(C₁₋₃-alkylene)-C(═O)—NH₂, —NH—(C₁₋₃-alkylene)-C(═O)—NHR^{Y7}, —NH—(C₁₋₃-alkylene)-C(═O)—NR^{Y7}R^{Y8}, or oxo (═O), wherein 1 or 2 non-adjacent CH₂ groups of the C₁₋₆-alkyl radical may independently from each other be replaced by O, S, N(H) or N—R⁷ and/or 1 or 2 non-adjacent CH groups of the C₁₋₆-alkyl radical may independently from each other be replaced by N;
LA^{Z} denotes a divalent straight-chain or branched C₁₋₆-alkylene radical which alkylene radical may be unsubstituted or mono-, di- or trisubstituted with independently from each other HaI, —CN, —NO₂, —SF₅, —SO₂NH₂, —SO₂NHR^{Z7}, —SO₂NR^{Z7}R^{Z8}, —NH—SO₂—R^{Z9}, —NR^{Z7}—SO₂—R^{Z9}, —S—R^{Z9}, —S(═O)—R^{Z9}, —SO₂—R^{Z9}, —NH₂, —NHR^{Z7}, —NR^{Z7}R^{Z8}, —OH, —O—R^{Z9}, —CHO, —C(═O)—R^{Z9}, —COOH, —C(═O)—O—R^{Z9}, —C(═O)—NH₂, —C(═O)—NHR^{Z7}, —C(═O)—NR^{Z7}R^{Z8}, —NH—C(═O)—R^{Z9}, —NR^{Z7}—C(═O)—R^{Z9}, —NH—(C₁₋₃-alkylene)-C(═O)—NH₂, —NH—(C₁₋₃-alkylene)-C(═O)—NHR^{Z7}, —NH—(C₁₋₃-alkylene)-C(═O)—NR^{Z7}R^{Z8}, or oxo (═O), wherein 1 or 2 non-adjacent CH₂ groups of that divalent alkylene radical may be replaced independently from each other by 0, S, —N(H) or N—R^{Z7} and/or 1 or 2 non-adjacent CH groups of that divalent alkylene radical may be replaced by N;
R^{W4}, R^{W5}, and R^{W6} denote Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, or CA^{X}
or
R^{W4} and R^{W5} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X7}, R^{X8}, R^{X9}, R^{Y7}, R^{Y8}, R^{Y9}, R^{Z7}, R^{Z8}, and R^{Z9} denote independently from each other straight-chain or branched C₁₋₆-alkyl, which may be unsubstituted or mono-, di- or trisubstituted with independently from each other HaI, —CN, —NO₂, —SF₅, —SO₂NH₂, —SO₂NHR^{X7v}, —SO₂NR^{X7v}R^{X8v}, —NH—SO₂—R^{X9v}, —NR^{X7v}—SO₂—R^{X9v}, —S—R^{X9v}, —S(═O)—R^{X9v}, —SO₂—R^{X9v}, —NH₂, —NHR^{X7v}, —NR^{X7v}R^{X8v}—OH, —O—R^{X9v}, —CHO, —C(═O)—R^{X9v}, —COOH, —C(═O)—O—R^{X9v}, —C(═O)—NH₂, —C(═O)—NHR^{X7v}, —C(═O)—NR^{X7v}R^{X8v}, —NH—C(═O)—R^{X9v}, —NR^{X7v}—C(═O)—R^{X9v}, —NH—(C₁₋₃-alkylene)-C(═O)—NH₂, —NH—(C₁₋₃-alkylene)-C(═O)—NHR^{X7v}, —NH—(C₁₋₃-alkylene)-C(═O)—NR^{X7v}R^{X8v}, or oxo (═O), wherein 1 or 2 non-adjacent CH₂ groups of the C₁₋₆-alkyl radical may independently from each other be replaced by O, S, N(H) or N—R^{X7v} and/or 1 or 2 non-adjacent CH groups of the C₁₋₆-alkyl radical may independently from each other be replaced by N, or a saturated monocyclic carbocycle with 3, 4, 5, 6, or 7 carbon atoms, which may be unsubstituted or mono- or disubstituted with independently from each other with HaI, Ar^{X}, Ar^{X}_Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, —CN, —NO₂, —SF₅, —SO₂NH₂, —SO₂NHR^{X7v}—SO₂NR^{X7v}R^{x8v}, —NH—SO₂—R^{X9v}, —NR^{X7v}—SO₂—R^{X9v}, —S—R^{X9v}, —S(═O)—R^{X9v}, —SO₂—R^{X9v}, —NH₂, —NHR^{X7v}—NR^{X7v}R^{X8v}—OH, —O—R^{X9v}, —CHO, —C(═O)—R^{X9v}, —COOH, —C(═O)—O—R^{X9v}, —C(═O)—NH₂, —C(═O)—NHR^{X7v}, —C(═O)—NR^{X7v}R^{X8v}, —NH—C(═O)—R^{X9v}, —NR^{X7v}—C (═O)—R^{X9v}, —NH—(C₁₋₃-alkylene)-C(═O)—NH₂, —NH—(C₁₋₃-alkylene)-C(═O)—NHR^{X7v}, —NH—(C₁₋₃-alkylene)-C(═O)—NR^{X7v}R^{X8v}, or oxo (═O), with the proviso that if any of the substituents of that monocyclic carbocycle is Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, or LA^{Z}-Hetcyc^{Y}, then any radical R^{X7}, R^{X8}, R^{X9}, R^{Y7}, R^{Y8}, R^{Y9}, R^{Z7}, R^{Z8} and R^{Z9} of any substituent of Ar^{X}, Ar^{Y}, Hetar^{X}, Hetar^{Y}, Hetcyc^{X}, Hetcyc^{Y}, LA^{X} and LA^{Z} may not denote a mono- or disubstituted monocyclic carbocycle, or a saturated monocyclic heterocycle with 3, 4, 5, 6, or 7 ring atoms wherein 1 or 2 ring atom(s) is/are heteroatom(s) selected from N, O and/or S and the remaining ring atoms are carbon atoms, wherein that heterocycle may be unsubstituted or substituted with straight-chain or branched C₁₋₆-alkyl, —C(═O)—C₁₋₆-alkyl (straight-chain or branched) and/or oxo (═O), or a phenyl, —CH₂-phenyl, -naphthyl, —CH₂-naphthyl, heteroaromatic ring system or —CH₂-heteroaromatic ring system with 5, 6, 7, 8, 9, 10, or 11 ring atoms, wherein 1, 2, 3, 4, or 5 of said ring atoms of said heteroaromic ring system is/are hetero atom(s) selected from N, O and/or S and the remaining are carbon atoms, wherein said phenyl, naphthyl or heteroaromatic ring system may be unsubstituted or mono-, di- or trisubstituted with independently from each other straight-chain or branched C₁₋₆-alkyl or —O—C₁₋₆-alkyl, HaI or —C(═O)—C₁₋₆-alkyl (straight-chain or branched);
or
each pair R^{X7} and R^{X8}; R^{Y7} and R^{Y8}; and R^{Z7} and R^{Z8} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X7v}, R^{X8v}, and R^{X9v} denote independently from each other straight-chain or branched C₁₋₆-alkyl, which may be unsubstituted or mono-, di- or trisubstituted with HaI, or a unsubstituted saturated monocyclic carbocycle with 3, 4, 5, 6, or 7 carbon atoms;
or
R^{X7v} and R^{X8}V form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
CA^{X} and CA^{Y} denote independently from each other a saturated monocyclic carbocycle with 3, 4, 5, 6, or 7 carbon atoms, wherein the carbocycle may be unsubstituted or mono- or disubstituted with independently from each other R^{CA1} or R^{CA2};
R^{CA1} and R^{CA2} denote independently from each other H, HaI, Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{X}, LA^{Z}-Ar^{Y}, LA^{Z}-Hetar^{Y}, LA^{Z}-Hetcyc^{Y}, —CN, —NO₂, —SF₅, —SO₂NH₂, —SO₂NHR^{X7}, —SO₂NR^{X7}R^{X8}, —NH—SO₂—R^{X9}, —NR^{X7}—SO₂—R^{X9}, —S—R^{X9}, —S(═O)—R^{X9}, —SO₂—R^{X9}, —NH₂, —NHR^{X7}, —NR^{X7}R^{X8}, —OH, —O—R^{X9}, —CHO, —C(═O)—R^{X9}, —COOH, —C(═O)—O—R^{X9}, —C(═O)—NH₂, —C(═O)—NHR^{X7}, —C(═O)—NR^{X7}R^{X8}, (═O)—R^{X9}, —NR^{X7}—C(═O)—R^{X9}, —NH—(C₁₋₃-alkylene)-C(═O)—NH₂, —NH—(C₁₋₃-alkylene)-C(═O)—NHR^{X7}, —NH—(C₁₋₃-alkylene)-C(═O)—NR^{X7}R^{X8}, or oxo (═O), with the proviso that if R^{CA1} or R^{CA2} denotes Ar^{X}, Ar^{X}—Ar^{Y}, Ar^{X}-Hetar^{Y}, Ar^{X}-Hetcyc^{Y}, Ar^{X}-LA^{Z}-Ar^{Y}, Ar^{X}-LA^{Z}-Hetar^{Y}, Ar^{X}-LA^{Z}-Hetcyc^{Y}, Hetar^{X}, Hetar^{X}-Ar^{Y}, Hetar^{X}-Hetar^{Y}, Hetar^{X}-Hetcyc^{Y}, Hetar^{X}-LA^{Z}-Ar^{Y}, Hetar^{X}-LA^{Z}-Hetar^{Y}, Hetar^{X}-LA^{Z}-Hetcyc^{Y}, Hetcyc^{X}, Hetcyc^{X}-Ar^{Y}, Hetcyc^{X}-Hetar^{Y}, Hetcyc^{X}-Hetcyc^{Y}, Hetcyc^{X}-LA^{Z}-Ar^{Y}, Hetcyc^{X}-LA^{Z}-Hetar^{Y}, Hetcyc^{X}-LA^{Z}-Hetcyc^{Y}, LA^{Z}-Ar^{Y}, LA^{Z}-Hetar^{Y}, or LA^{Z}-Hetcyc^{Y}, then Ar^{X}, Ar^{Y}, Hetar^{X}, Hetar^{Y}, Hetcyc^{X}, and Hetcyc^{Y} may not be substituted with CA^{X} or CA^{Y};
HaI denotes F, Cl, Br, or I;
or derivatives, N-oxides, prodrugs, solvates, tautomers or stereoisomers thereof as well as the physiologically acceptable salts of each of the foregoing, including mixtures thereof in all ratios.

2. The Compound according to claim 1, or derivatives, N-oxides, prodrugs, solvates, tautomers or stereoisomers thereof as well as the physiologically acceptable salts of each of the foregoing, including mixtures thereof in all ratios,
wherein
X denotes N—R⁵ or O;
R¹ denotes Ar^{X}, Hetar^{X}, Ar^{X}—Ar^{Y}, or Ar^{X}-Hetar^{Y};
R² and R³ both denote H;
R⁴ denotes Ar^{W} or Hetar^{W}, which Ar^{W} or Hetar^{W} has in its ortho-position, relative to the attachment of R⁴ to X, one substituent R^{W1} and may or may not bear further substituents;
R⁵ denotes H or LA^{X};
Ar^{W} denotes a monocyclic aromatic ring system with 6 ring carbon atoms which ring system may bear, besides the ortho-substituent R^{W1}, no further substituent or one further substituent R^{W2}, wherein R^{W1} and R^{W2} may be the same or different;
Ar^{X} denotes a monocyclic aromatic ring system with 6 ring carbon atoms which ring system may be unsubstituted or mono- or di-substituted with independently from each other R^{X1} or R^{X2};
Ar^{X} denotes a monocyclic aromatic ring system with 6 ring carbon atoms which ring system may be unsubstituted or mono- or di-substituted with independently from each other R^{Y1} or R^{X2},
Hetar^{W} denotes a monocyclic aromatic ring system with 5 or 6 ring atoms wherein 1, 2 or 3 of said ring atoms is/are nitrogen atom(s) and the remaining are carbon atoms, wherein ring system may bear, besides the ortho-substituent R^{W1}, further substituent or one further substituent R^{W2} wherein R^{W1} and R^{W2} may be the same or different;
Hetar^{X} denotes a mono- or bi-cyclic aromatic ring system with 5, 6, 9, or 10 ring atoms wherein 1, 2, 3 or 4 of said ring atoms is/are a hetero atom(s) selected from N, O and/or S and the remaining are carbon atoms, wherein aromatic ring system may be unsubstituted or mono- or di-substituted with independently from each other R^{X1} or R^{X2};
Hetar^{Y} denotes a monocyclic aromatic ring system with 5 or 6 ring atoms wherein 1, 2 or 3 of said ring atoms is/are a nitrogen atom(s) and the remaining are carbon atoms, wherein that aromatic ring system may be unsubstituted or mono-substituted with R^{Y1};
Hetcyc^{X} denotes a saturated mono-cyclic heterocycle with 4, 5, 6, or 7, ring atoms wherein 1 or 2 ring atom(s) is/are heteroatom(s) selected from N, O and/or S and the remaining ring atoms are carbon atoms, wherein that heterocycle may be unsubstituted or mono-, disubstituted or trisubstituted with independently from each other R^{X4}, R^{X5}, or R′;
Hetcyc^{Y} denotes a saturated monocyclic heterocycle with 4, 5, 6, or 7 ring atoms wherein 1 or 2 ring atom(s) is/are heteroatom(s) selected from N, O and/or S and the remaining ring atoms are carbon atoms, wherein that heterocycle may be unsubstituted or mono-, di- or trisubstituted with independently from each other R^{Y4}, R^{Y5}, or R^{Y6};
R^{W1} denotes LA Hetar^{X}, Hetcyc^{X}, HaI, —CN, —OH, —O—R^{W6}, —SO₂NH₂, —SO₂NHR^{W4}, —SO₂NR^{W4}R^{W5}, —NH—SO₂—R^{W6}, —NR^{W4}—SO₂—R^{W6}, —SO₂—R^{W6}, —NH₂, —NHR^{W4}, —NR^{W4}R^{W5}, —C(═O)—OH, —C(═O)—O—R^{W6}, —C(═O)—NH₂, —C(═O)—NHR^{W4}, —C(═O)—NR^{W4}R^{W5}, —NH—C(═O)—R^{W6}, or —NR^{W4}—C(═O)—R^{W6};
or R⁵ and R^{W1} form together a divalent alkylene chain with 1, 2, or 3 chain carbon atoms;
R^{W2} denotes H, Hetar^{X}, Hetcyc^{X}, HaI, LA^{X}, —CN, —OH, —O—R^{W6}, —NO₂—NH₂, —NHR^{W4}, —NR^{W4}R^{W5}, —COOH, —C(═O)—O—R^{W6}, —C(═O)—NH₂, —C(═O)—NHR^{W4}, —C(═O)—NR^{W4}R^{W5}, —C(═O)—NH—NH₂, —NH—C(═O)—R^{W6}, or —NR^{W4}—C(═O)—R^{W6};
or R^{W1} and R^{W2} form together a divalent alkylene chain with 3, 4, or 5 chain carbon atoms wherein 1 or 2 of non-adjacent CH₂ groups of the divalent alkylene chain may be replaced independently from each other by —N(H)—, —N(C₁₋₆-alkyl)-, —N(—C(═O)—C₁₋₄-alkyl), or —O—, wherein the C₁₋₆-alkyl and C₁₋₄-alkyl radicals may be straight-chain or branched — and wherein 2 adjacent CH₂ groups may together be replaced by a —CH═CH— moiety, which divalent alkylene chain may be unsubstituted or mono- or di-substituted with independently from each other straight-chain or branched —C₁₋₆-alkyl or ═O (oxo);
R^{X1} and R^{X2} denote independently from each other H, LA^{X}, —NH₂, —NHR^{X7}, —NR^{X7}R^{X8}, HaI, —OH, —OR^{X9}, —SR^{X9}, —SF₅, —C(═O)—NH₂, —C(═O)—NHR^{X7}, —C(═O)—NR^{X7}, —C(═O)—NR^{X7}R^{X8}, or —NH—C(═O)—R^{X9},
or form a divalent alkylene chain with 3, 4, or 5 chain carbon atoms wherein 1 or 2 non-adjacent CH₂ group(s) of the divalent alkylene chain may be replaced independently from each other by —O—, which divalent alkylene chain may be unsubstituted or mono- or di-substituted with independently from each other straight-chain or branched —C₁₋₆-alkyl;
R^{Y1} and R^{Y2} denote independently from each other LA^{Y};
LA^{X} denotes straight-chain or branched C₁₋₆-alkyl which may be unsubstituted or mono-, di- or trisubstituted with independently from each other HaI, —CN, —NH₂, —NHR^{X7}, or —NR^{X7}R^{X8};
LA^{Y} denotes straight-chain or branched C₁₋₆-alkyl;
LA^{Z} denotes a divalent straight-chain or branched C₁₋₆-alkylene radical;
R^{X4}, R^{X5}, and R^{X6} denote independently from each other H, HaI, LA^{X}, —C(═O)—R^{X9}, or oxo (═O);
R^{Y4}, R^{Y5}, and R^{Y6} denote independently from each other H, HaI, LA^{Y}, —C(═O)—R^{Y9}, or oxo (═O);
R^{W4} denotes straight-chain or branched C₁₋₆-alkyl, saturated monocyclic carbocycle with 3, 4, 5, 6, or 7 carbon atoms, Ar^{X}, Hetar^{X}, Hetcyc^{X}, LA^{Z}-Ar^{Y}, LA^{Z}-Hetar^{Y} or LA^{Z}-Hetcyc^{Y};
R^{W5} and R^{W6}, denote independently from each other straight-chain or branched C₁₋₆-alkyl, a saturated monocyclic carbocycle with 3, 4, 5, 6, or 7 carbon atoms, Ar^{X}, Hetar^{X}, Hetcyc^{X}, LAZ-ArY, LAZ-HetarY or LAZ-HetcycY
or
R^{W4} and R^{W5} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
R^{X7}, R^{X8}, R^{X9}, and R^{Y9} denote independently from each other straight-chain or branched C₁₋₆-alkyl, which may be unsubstituted or mono-, di- or trisubstituted with HaI or monosubstituted with NH₂, a saturated monocyclic carbocycle with 3, 4, 5, 6, or 7 carbon atoms, or a saturated monocyclic heterocycle with 3, 4, 5, 6, or 7 ring atoms wherein 1 or 2 ring atom(s) is/are heteroatom(s) selected from N, O and/or S and the remaining ring atoms are carbon atoms, wherein that heterocycle may be unsubstituted or substituted with straight-chain or branched C₁₋₆-alkyl, —C(═O)—C₁₋₆-alkyl (straight-chain or branched) and/or oxo (═O), or a phenyl, —CH₂-phenyl, -naphthyl, —CH₂-naphthyl, heteroaromatic ring system or —CH₂-heteroaromatic ring system with 5, 6, 7, 8, 9, 10, or 11 ring atoms, wherein 1, 2, 3, 4, or 5 of said ring atoms of said heteroaromic ring system is/are hetero atom(s) selected from N, O and/or S and the remaining are carbon atoms, wherein said phenyl, naphthyl or heteroaromatic ring system may be unsubstituted or mono-, di- or trisubstituted with independently from each other straight-chain or branched C₁₋₆-alkyl or O—C₁₋₆-alkyl, HaI or C(═O)—C₁₋₆-alkyl (straight-chain or branched)
or
R^{X7} and R^{X8} form together with the nitrogen atom to which they are attached to a 3, 4, 5, 6 or 7 membered heterocycle wherein heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
HaI denotes F, Cl, Br, I.

3. The compound according to claim 1, or derivatives, N-oxides, prodrugs, solvates, tautomers or stereoisomers thereof as well as the physiologically acceptable salts of each of the foregoing, including mixtures thereof in all ratios,
wherein
X denotes N—R⁵ or O;
R¹ denotes Ar^{X1} or Hetar^{X1};
R⁵ denotes H;
Ar^{X1} denotes phenyl which may be unsubstituted or mono-substituted with R^{X1a} or di-substituted with independently from each other R^{X1a} or R^{X2a};
Hetar^{X1} denotes a bicyclic aromatic ring system with 9 ring atoms wherein (i) 1 of said ring atoms is a nitrogen atom or an oxygen atom or a sulfur atom and the remaining are carbon atoms; or (ii) 1 of said ring atoms is a nitrogen atom and 1 further of said ring atoms is an oxygen atom or a sulfur atom, wherein that further hetero atom may be adjacent or not adjacent to the nitrogen atom, and the remaining are carbon atoms; or (iii) 2 of said ring atoms are nitrogen atoms and the remaining are carbon atoms; or (iv) 2 of said ring atoms are nitrogen atoms and another of said ring atoms is an oxygen atom or a sulfur atom and the remaining are carbon atoms; or (v) 3 of said ring atoms are nitrogen atoms and the remaining are carbon atoms; wherein that aromatic ring system may be unsubstituted or mono-substituted with R^{X1b} or di-substituted with independently from each other R^{X1b} or R^{X2b};
R^{X1a} and R^{X2b} denote independently from each other straight-chain or branched C₁₋₆-alkyl, which C₁₋₆-alkyl may be unsubstituted or mono-, di- or trisubstituted with F and/or Cl, straight-chain or branched O—C₁₋₆-alkyl, wherein —O—C₁₋₆-alkyl may be unsubstituted or mono-, di- or trisubstituted with F and/or Cl, —OH, —SR^{X9}, —SF₅, F, Cl, Br, —NH₂, —NHR^{X7}, —NR^{X7}R^{X8}, —C(═O)—NH₂, —C(═O)—NHR^{X7}, —C(═O)—NR^{X7}R^{X8} or form together a —CH₂—CH₂—O—, a —OCH₂—CH₂—O— or a —OCH₂—C(CH₃)₂— chain;
R^{X1b} and R^{X2b} denote independently from each other straight-chain or branched C₁₋₆-alkyl, which C₁₋₆-alkyl may be unsubstituted or mono-, di- or trisubstituted with F and/or Cl, Cl, Br, F, —OH, —NH₂, —NHR^{X7}, —NR^{X7}R^{X8}, —NH—C(═O)-methyl, —NH—C(═O)—CH₂—NH₂, or —NH—C(═O)-pyrrolidin-2-yl;
R^{X7}, R^{X8}, and R^{X9} denote independently from each other straight-chain or branched C₁₋₆-alkyl or a saturated monocyclic carbocycle with 3, 4, 5, 6, or 7 carbon atoms
or
R^{X7} and R^{X8} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl.

4. The Compound according to claim 1, or derivatives, N-oxides, prodrugs, solvates, tautomers or stereoisomers thereof as well as the physiologically acceptable salts of each of the foregoing, including mixtures thereof in all ratios, wherein R¹ denotes methylphenyl, 3-methylphenyl, ethylphenyl, 3-ethylphenyl, 4-ethylphenyl, trifluoromethylphenyl, 4-(trifluoromethyl)phenyl, dimethylphenyl, 2,5-dimethylphenyl, diethylphenyl, 3,5-diethylphenyl, methoxyphenyl, 3-methoxyphenyl, 4-methoxyphenyl, trifluoromethoxyphenyl, 3-trifluoromethoxyphenyl, methyl sulfanylphenyl, 3-methylsulfanylphenyl, pentafluorosulfanylphenyl, 4-pentafluoro-λ⁶-sulfanylphenyl, methoxy-methylphenyl (methoxy-tolyl), 2-methoxy-5-methylphenyl, 5-methoxy-2-methylphenyl, fluorophenyl, 4-fluorophenyl, bromophenyl, 3-bromophenyl, 4-bromophenyl, bromo-fluorophenyl, 4-bromo-3-fluorophenyl, bromo-methylphenyl, 4-bromo-2-methylphenyl, chloro-methoxyphenyl, 2-chloro-5-methoxy-phenyl, aminophenyl, 3-aminophenyl, 4-aminophenyl, amino-methylphenyl, 2-amino-5-methylphenyl, 3-amino-4-methylphenyl, amino-fluoro-phenyl, 4-amino-3-fluorophenyl, hydroxy-methylphenyl, 2-hydroxy-5-methylphenyl, dihydrobenzofuran-5-yl, indolyl, 1H-indol-6-yl, N-methyl-indol-6-yl, 1-ethyl-1H-indol-6-yl (N-ethyl-indol-6-yl), 1-n-propyl-indol-6-yl, N-isopropyl-indol-6-yl, difluoromethyl-indol-6-yl, 2-(difluoromethyl)-1H-indol-6-yl, dimethylindolyl, dimethylindol-6-yl, 1,4-dimethyl-1H-indol-6-yl, 1,5-dimethyl-1H-indol-6-yl, fluoro-methylindolyl, fluoro-1-methylindol-6-yl, 4-fluoro-1-methylindol-6-yl, 5-fluoro-1-methylindol-6-yl, 7-fluoro-1-methyl-indol-6-yl, dimethylaminophenyl, 3-N,N-dimethylaminophenyl, dimethylamino-methylphenyl, 2-dimethylamino-5-methylphenyl, benzothiazolyl, benzothiazol-6-yl, benzothiazol-5-yl, dimethyldihydrobenzofuranyl, 3,3-dim ethyl-2,3-dihydro-1-benzofuran-5-yl, methylbenzofuranyl, methyl-benzofuran-5-yl, 3-methyl-benzofuran-5-yl, benzothiophenyl, benzothiophen-5-yl, methylbenzothiophenyl, 3-methyl-1-benzothiophen-5-yl, trifluoromethyl-benzothiophenyl, 3-(trifluoromethyl)-1-benzothiophen-5-yl, aminobenzothiophenyl, 2-amino-1-benzothiophen-5-yl, 2-amino-1-benzothiophen-6-yl, 2-(acetylamino)-1-benzothiophen-5-yl, 2-(NH₂—CH₂—C(═O)NH-)-1-benzothiophen-5-yl, 2,3-dihydrobenzo[1,4]dioxin-6-yl, 1-methyl-1H-pyrrol o[2,3-b]pyrdin-6-yl, 1,2-benzothiazol-5-yl, 1,3-benzothiazol-5-yl, 1,3-benzothiazol-6-yl, 2-amino-1,3-benzothiazol-5-yl, 2-amino-1,3-benzothiazol-6-yl, 2-methylamino-1,3-benzothiazol-5-yl, 2-dimethylamino-1,3-benzothiazol-5-yl, 2-(acetylamino)-1,3-benzothiazol-5-yl, 2-(pyrrolidin-2-yl-C(═O)—NH-)-1,3-benzothiazol-5-yl, 2-(pyrrolidin-2-yl-C(═O)—NH-)-1,3-benzothiazol-6-yl, benzothiazololyl (hydroxybenzothiazolyl, dihydro-benzothiazolonyl), 1,3-benzothiazol-2-ol-5-yl (2-hydroxy-1,3-benzothiazol-5-yl, 2,3-dihydro-1,3-benzothiazol-2-on-5-yl), benzoxadiazolyl, 2,1,3-benzoxadiazol-5-yl, benzothiadiazolyl, 2,1,3-benzothiadiazol-5-yl, benzotriazolyl, or 1,2,3-benzotriazol-5-yl.

5. The compound according to claim 1, or derivatives, N-oxides, prodrugs, solvates, tautomers or stereoisomers thereof as well as the physiologically acceptable salts of each of the foregoing, including mixtures thereof in all ratios,
wherein
R⁴ denotes Ar^{W4} or Hetar^{W4};
Ar^{W4} denotes phenyl which is substituted with R^{W1}a in the ortho-position, relative to the attachment of Ar^{W4} to X, may bear no further substituent or one further substituent R^{W2}a;
Hetar^{W4} denotes a monocyclic aromatic ring system with 5 or 6 ring atoms wherein 1, 2 or 3 of said ring atoms is/are nitrogen atom(s) and the remaining are carbon atoms, wherein that ring system is substituted with R^{W1b} in the ortho-position, relative to the attachment of Hetar^{W4} to X, and may bear no further substituent or one further substituent R^{W2b};
R^{W1a} and R^{W1b} denote independently from each other LA^{X}a, Hetar^{X4}, Hetcyc^{X4}, HaI, —CN, —OH, —O—R^{W6a}, —SO₂NH₂, —SO₂NHR^{W4a}, —SO₂NR^{W4a}R^{W5a}, —SO₂—R^{W6a}, —NH₂, —NHR^{W4a}—NR^{W4a}R^{W5a}—C(═O)—OH, C(═O)—O—R^{W6a}, —C(═O)—NH₂, —C(═O)—NHR^{W4a}, or
—C(═O)—NR^{W4a}R^{W5a};
R^{W2a} and R^{W2b} denote independently from each other H, HaI, LA^{Xa}, —CN, —NO₂, —NH₂, —NHR^{W4b}—NR^{W4b}R^{W5b}—C(═O)—O—R^{W6b}, —C(═O)—NH₂, —C(═O)—NHR^{W4b}, —C(═O)—NR^{W4b}R^{W5b}, —C(═O)—NH—NH₂, —NH—C(═O)—R^{W6b}, Hetar^{X4}, or Hetcyc^{X4}; or
R^{W1a} and R^{W2a} or R^{W1b} and R^{W2b} form together a divalent alkylene chain with 3 or 4 chain carbon atoms wherein 1 or 2 of non-adjacent CH₂ groups of the divalent alkylene chain may be replaced independently from each other by N(H)—, —N(C₁₋₆-alkyl)-, —N(—C(═O)—C₁₋₄-alkyl), or —O—, wherein the C₁₋₆-alkyl and C₁₋₄-alkyl radicals may be straight-chain or branched —, which divalent alkylene chain may be unsubstituted or mono- or di-substituted with independently from each other straight-chain or branched —C₁₋₆-alkyl;
Ar^{X4} denotes a monocyclic aromatic ring system with 6 ring carbon atoms which ring system may be unsubstituted or monosubstituted with LA^{X4};
Hetar^{X4} denotes monocyclic aromatic ring system with 5 or 6 ring atoms wherein 1, 2, 3 or 4 of said ring atoms is/are a nitrogen atom(s) and the remaining are carbon atoms, wherein that aromatic ring system may be unsubstituted or mono-substituted with LA^{X4}, —NH₂, —NHR^{X7a}, or —NR^{X7a}R^{X8a},
Hetar^{Y4} denotes a monocyclic aromatic ring system with 5 or 6 ring atoms wherein 1, 2 or 3 of said ring atoms is/are a nitrogen atom(s) and the remaining are carbon atoms, wherein that aromatic ring system may be unsubstituted or mono-substituted with LA^{Y4};
Hetcyc^{X4} denotes a saturated mono-cyclic heterocycle with 4, 5 or 6 ring atoms wherein 1 or 2 ring atom(s) is/are heteroatom(s) selected from N, O and/or S and the remaining ring atoms are carbon atoms, wherein that heterocycle may be unsubstituted or monosubstituted with LA^{X4} or C(═O)-LA^{X4} or oxo (═O) or disubstituted with oxo (═O) and LA^{X4} or HaI and LA^{X4} or trisubstituted with one of two HaI and one or two LA^{X4};
Hetcyc^{Y4} denotes a saturated mono-cyclic heterocycle with 4, 5 or 6 ring atoms wherein 1 or 2 ring atom(s) is/are heteroatom(s) selected from N, O and/or S and the remaining ring atoms are carbon atoms, wherein that heterocycle may be unsubstituted or monosubstituted with LA^{Y4} or C(═O)-LA^{Y4} or oxo (═O) or disubstituted with oxo (═O) and LA^{Y4};
LA^{Xa} denotes straight-chain or branched C₁₋₆-alkyl which may be unsubstituted or mono-, di- or trisubstituted with independently from each other HaI, —CN, —NH₂, —NHR^{X7a}, or —NR^{X7a}R^{X8a};
LA^{X4} and LA^{Y4} denote independently from each other straight-chain or branched C₁₋₆-alkyl;
LA^{Z4} denotes a straight-chain or branched divalent C₁₋₆-alkylene radical;
R^{W4a}, R^{W5a}, R^{W6a}, R^{W4b}, R^{W5b}, and R^{W6b} denote independently from each other straight-chain or branched C₁₋₆-alkyl, a saturated monocyclic carbocycle with 3, 4, 5, 6, or 7 carbon atoms, Ar^{X4}, Hetar^{X4}, Hetcyc^{X4}, LA^{Z4}-Hetar^{Y4} or LA^{Z4}-Hetcyc^{Y4};
R^{X7a} and R^{X8a} denote independently from each other straight-chain or branched C₁₋₆-alkyl or a saturated monocyclic carbocycle with 3, 4, 5, 6, or 7 carbon atoms or a monocyclic aromatic ring system with 5 or 6 ring atoms wherein 1, 2, 3 or 4 of said ring atoms is/are a nitrogen atom(s) and the remaining are carbon atoms, wherein that aromatic ring system may be unsubstituted or mono-substituted with straight-chain or branched C₁₋₆-alkyl;
or
each pair R^{W4}a and R^{W5}a; R^{W4b} and R^{W5b}; R^{X7a} and R^{X8a} form together with the nitrogen atom to which they are attached to a 3, 4, 5, 6 or 7 membered heterocycle wherein that heterocycle may not contain any further heteroatom or may contain besides said nitrogen atom one further hetero ring atom selected from N, O and S, wherein, if that further hetero atom is N, that further N may be substituted with H or straight-chain or branched C₁₋₆-alkyl;
HaI denotes F, Cl, Br, I.

6. The compound according to claim 5, or
derivatives, N-oxides, prodrugs, solvates, tautomers or stereoisomers thereof as well as the physiologically acceptable salts of each of the foregoing, including mixtures thereof in all ratios,
wherein
Ar^{W4} denotes phenyl which is substituted with R^{W1a} in the ortho-position, relative to the attachment of Ar^{W4} to X, and bears no further substituent;
Hetar^{W4} denotes a monocyclic aromatic ring system with 6 ring atoms wherein 1 or 2 of said ring atoms is/are nitrogen atom(s) and the remaining are carbon atoms, wherein that ring system is substituted with R^{W1b} in the ortho-position, relative to the attachment of Hetar^{W4} to X, and bears no further substituent.

7. The compound according to claim 5, or
derivatives, N-oxides, prodrugs, solvates, tautomers or stereoisomers thereof as well as the physiologically acceptable salts of each of the foregoing, including mixtures thereof in all ratios,
wherein
Ar^{W4} denotes phenyl which is substituted with R^{W1a} in the ortho-position, relative to the attachment of Ar^{W4} to X, and bears one further substituent R^{W2a} in para-position relative to R^{W1a};
Hetar^{W4} denotes a monocyclic aromatic ring system with 6 ring atoms wherein 1 or 2 of said ring atoms is/are nitrogen atom(s) and the remaining are carbon atoms, wherein that ring system is substituted with R^{W1b} in the ortho-position, relative to the attachment of Hetar^{W4} to X, and bears one further substituent R^{W2b} in para-position relative to R^{W1b}.

8. The compound according to claim 5, or derivatives, N-oxides, prodrugs, solvates, tautomers or stereoisomers thereof as well as the physiologically acceptable salts of each of the foregoing, including mixtures thereof in all ratios,
wherein
R^{W1a} and R^{W1b} denote independently from each other methyl, methylaminomethyl, (dimethylamino)methyl, pyrazolyl, methylpyrazolyl, imidazolyl, methylimidazolyl, 1-methyl-1H-imidazol-4-yl, pyrimidinyl, tetrazolyl, 1H-1,2,3,4-tetrazol-5-yl, Cl, —CN, —SO₂NH₂, —SO₂NH(CH₃), —SO₂N(CH₃)₂, —SO₂—N-morpholinyl, —SO₂—N-piperazinyl, —SO₂—CH₃, —SO₂—NH-pyrrolidinyl, —SO₂—NH-pyrrolidin-3-yl, —SO₂—NH-methylpyrrolidinyl, —SO₂—NH-(1-methylpyrrolidin-3-yl), —SO₂—NH-(piperdinyl), —SO₂—NH-(piperdin-3-yl), —SO₂—NH-(methylpiperdinyl), —SO₂—NH-(1-methylpiperdin-3-yl), —SO₂—NH-oxanyl, —SO₂—NH-oxan-3-yl, —SO₂—NH—CH₂-(pyrrolidinyl), —SO₂—NH—CH₂-(pyrrolidin-3-yl), —SO₂—NH—CH₂-(methylpyrrolidinyl), —SO₂—NH—CH₂-(1-methylpyrrolidin-3-yl), —SO₂—NH—CH₂-oxanyl, —SO₂—NH—CH₂-oxan-4-yl, —SO₂—NH—CH₂-pyrazolyl, —SO₂—NH—CH₂-pyrazol-4-yl, —SO₂—NH—CH₂-(methylpyrazolyl), —SO₂—NH—CH₂-(1-methyl-1H-pyrazol-4-yl), —SO₂—NH-(pyrimidin-5-yl), —SO₂—NH—CH₂-(pyrimidin-5-yl), —SO₂—N(CH₃)—CH₂-(pyrimidin-5-yl), —NH₂, —N-piperazinyl, —N-4-methylpiperazinyl, 4-N-acetylpiperazin-1-yl, —OH, —OCH₃, —C(═O)—OH, —C(═O)—O-(n-C₄H₉), —C(═O)—O-pyrimidinyl, —C(═O)—O-pyrimidin-4-yl, —C(═O)—O-(aminopyrimidinyl), —C(═O)—O-(2-aminopyrimidin-4-yl), —C(═O)—NH₂, —C(═O)—NHCH₃, —C(═O)—N(CH₃)₂, —C(═O)—NH-cyclohexyl, —C(═O)—NH-phenyl, —C(═O)—NH-(azetidinyl), —C(═O)—NH-(methylazetidinyl), —C(═O)—NH-(1-methylazetidin-3-yl), —C(═O)—NH-(1-acetylazetidin-3-yl), —C(═O)—NH—CH₂-(azetidinyl), —C(═O)—NH—CH₂-(1-acetylazetidin-3-yl), —C(═O)—NH-(methylpyrrolidinyl), —C(═O)—NH-(1-methyl-pyrrolidin-3-yl), —C(═O)—NH-((3S)-1-methyl-pyrrolidin-3-yl), —C(═O)—NH-((3R)-1-methyl-pyrrolidin-3-yl), —C(═O)—N(CH₃)-(methylpyrrolidinyl), —C(═O)—N(CH₃)-(1-methyl-pyrrolidin-3-yl), —C(═O)—NH—CH₂-(methylpyrrolidinyl), —C(═O)—NH—CH₂-(1-methyl-pyrrolidin-3-yl), —C(═O)—NH-(1-acetylpyrrolidin-3-yl), —C(═O)—NH-(fluoro-methylpyrrolidinyl), —C(═O)—NH-(2-fluoro-1-methylpyrrolidin-3-yl), —C(═O)—NH-(5-fluoro-1-methylpyrrolidin-3-yl), —C(═O)—NH-(difluoro-methylpyrrolidinyl), —C(═O)—NH-(5,5-difluoro-1-methylpyrrolidin-3-yl), —C(═O)—NH-(3,3-difluoro-1-methylpyrrolidin-3-yl), —C(═O)—NH-oxanyl, —C(═O)—NH-oxan-4-yl, —C(═O)—NH-piperidinyl, —C(═O)—NH-piperidin-4-yl, —C(═O)—NH-piperidin-3-yl, —C(═O)—NH-methylpiperidinyl, —C(═O)—NH-(1-methylpiperidin-4-yl), —C(═O)—NH-(1-methylpiperidin-3-yl), —C(═O)—NH-(acetylpiperdinyl), —C(═O)—NH-(1-acetylpiperidin-3-yl), —C(═O)—NH-(1-acetylpiperidin-4-yl), —C(═O)—NH-(oxopyrrolidinyl), —C(═O)—NH—(N-methyl-oxopyrrolidinyl), —C(═O)—NH-(5-oxopyrrolidin-3-yl), —C(═O)—NH-(2-oxopyrrolidin-3-yl), —C(═O)—NH-(1-methyl-5-oxopyrrolidin-3-yl), —C(═O)—NH-(1-methyl-2-oxopyrrolidin-3-yl), —C(═O)—NH-morpholinyl, —C(═O)—NH—CH₂-morpholinyl, —C(═O)—NH—CH₂-morpholin-2-yl, —C(═O)—NH—CH₂-morpholin-3-yl, —C(═O)—NH—CH₂-(methylmorpholinyl), —C(═O)—NH—CH₂-(4-methylmorpholin-2-yl), —C(═O)—NH—CH₂-(acetylmorpholinyl), —C(═O)—NH—CH₂-(4-acetylmorpholin-2-yl), —C(═O)—NH—CH₂-(4-acetylmorpholin-3-yl), —C(═O)—NH-(oxopiperidinyl), —C(═O)—NH-(2-oxopiperidin-4-yl), —C(═O)—NH-(methyl-oxopiperidinyl), —C(═O)—NH-(1-methyl-2-oxopiperidin-4-yl), —C(═O)—NH-(1-methyl-6-oxopiperidin-3-yl), —C(═O)—NH(pyrimindin-4-yl), —C(═O)—NH(pyrimindin-5-yl), —C(═O)—NHCH₂(pyrimindin-5-yl), —C(═O)—NH-imidazolyl, —C(═O)—NH-imidazol-5-yl, —C(═O)—NH-methylimidazolyl, —C(═O)—NH-(1-methyl-imidazol-5-yl), —C(═O)—NH—CH₂-imidazolyl, —C(═O)—NH—CH₂-imidazol-5-yl, —C(═O)—NH—CH₂-(methylimidazolyl), —C(═O)—NH—CH₂-(1-methyl-1H-imidazol-5-yl), —C(═O)—NH(methylpyrazolyl), —C(═O)—NH(1-methyl-1H-pyrazol-4-yl), —C(═O)—NHCH₂(1-methylpyrazol-4-yl), —C(═O)—NH₂-pyridinyl, —C(═O)—NH₂-pyridin-3-yl, —C(═O)—NH-pyridazinyl, —C(═O)—NH-pyridazin-3-yl, —C(═O)—NH—CH₂-pyridazinyl, —C(═O)—NH—CH₂-pyridazin-3-yl, —C(═O)—NH-pyrimidinyl, —C(═O)—NH-pyrimidin-4-yl, —C(═O)—NH-pyrimidin-5-yl, or —CH₂—NH-(pyrimidin-5-yl);
R^{W2a} and R^{W2b} denote, if present, independently from each other H, Br, —CH₂NH₂, —CN, —NO₂, —NH₂, —NH—C(═O)—CH₃, —C(═O)—O-methyl, —C(═O)—NH₂,
—C(═O)—NH—NH₂, 4-methylpiperazin-1-yl, 4-acetylpiperazin-1-yl, methylpyrazolyl,
1-methyl-1H-pyrazol-5-yl, 1H-imidazol-1-yl, oxazolyl, 1,3-oxazol-2-yl, or 2H-1,2,3,4-tetrazol-5-yl; or
R^{W1b} and R^{W2b} form together a divalent —O—CH₂—CH₂—NH— chain it being understood that the the oxygen atom of that chain is attached to the Hetar^{W4} substituent at the position of R^{W1b} while the —NH— part of that chain is attached to the Hetar^{W4} substituent at the position of R^{W2b} and next to R^{W1b}.

9. The compound according to claim 5, or derivatives, N-oxides, prodrugs, solvates, tautomers or stereoisomers thereof as well as the physiologically acceptable salts of each of the foregoing, including mixtures thereof in all ratios,
wherein
Ar^{W4} denotes 2-((dimethylamino)methyl)phenyl, 2-(C(═O)OH)phenyl, 2-methylsulfonylphenyl (2-methanesulfonylphenyl), 2-(morpholine-4-sulfonyl)phenyl, 2-hydroxyphenyl, 2-methoxyphenyl, 2-cyanophenyl, 2-aminosulfonylphenyl, 2-(N-methylaminosulfonyl)phenyl, 2-((1-methylpyrrolidin-3-yl)-NH—SO₂-)phenyl, 2-((1-methylpiperidin-3-yl)-NH—SO₂-)phenyl, 2-((oxan-3-yl)-NH—SO₂-)phenyl, 2-((1-methylpyrrolidin-3-yl)-CH₂—NH—SO₂-)phenyl, 2-(oxan-4-yl-CH₂—NH—SO₂-)phenyl, 2-((1-methyl-1H-pyrazol-4-yl)-CH₂—NH—SO₂-)phenyl, 2-((pyrimidin-5-yl)-CH₂—NH—SO₂-)phenyl, 2-((pyrimidin-5-yl)-CH₂—N(CH₃)—SO₂-)phenyl, 2-(N,N-dimethylaminosulfonyl)phenyl, 2-(NH₂—C(═O)-)phenyl (2-carbamoylphenyl), 2-((1-methylpyrrolidin-3-yl)-NH—C(═O)-)phenyl, 5-bromo-2-methanesulfonylphenyl, 2-(piperazine-1-sulfonyl)phenyl, 5-cyano-2-methanesulfonylphenyl, 2-methanesulfonyl-5-amino-phenyl, 2-methanesulfonyl-5-nitro-phenyl, 2-methanesulfonyl-5-aminomethyl-phenyl, 2-methanesulfonyl-5-carbamoylphenyl (2-methanesulfonyl-5-(NH₂—C(═O)-)phenyl), (2-methanesulfonyl-5-(NH₂—NH—C(═O)-)phenyl), 2-methanesulfonyl-5-(CH₃C(═O)NH)-phenyl, 2-methanesulfonyl-5-(4-acetylpiperazin-1-yl)-phenyl, 2-methanesulfonyl-5-(4-methylpiperazin-1-yl)-phenyl, 2-methanesulfonyl-5-(1,3-oxazol-2-yl)phenyl, methanesulfonyl-5-(2H-1,2,3,4-tetrazol-5-yl)phenyl, or 5-(1H-imidazol-1-yl)-2-methanesulfonylphenyl;
Hetar^{W4} denotes 4-(methylamino)methylpyridin-3-yl, 4-((dimethylamino)methyl)pyridin-3-yl, 2-methylsulfonylpyrdin-3-yl, 4-methylsulfonylpyridin-3-yl, 2-aminopyridin-3-yl, 4-(NH₂—C(═O))-pyridin-3-yl, 4-chloropyridin-3-yl, 4-cyanopyridin-3-yl, 2-hydroxy-pyridin-3-yl, 2-methoxy-pyridin-3-yl, 3-methanesulfonyl-pyrazin-2-yl, 3-methanesulfonyl-pyridin-2-yl, 4-(C(═O)OH)pyridin-3-yl, 4-(1-methyl-1H-pyrazol-4-yl)-pyridin-3-yl, 4-(4-methylpiperazin-1-yl)-pyridin-3-yl, 4-(4-N-acetylpiperazin-1-yl)pyridin-3-yl, 4-(1-methyl-1H-imidazol-4-yl)pyridin-3-yl, 4-(pyrimidin-5-yl)-pyridin-3-yl, 4-methoxypyridin-3-yl, 4-(1H-1,2,3,4-tetrazol-5-yl)pyridin-3-yl, 4-((2-aminopyrimidin-4-yl)-O—C(═O))-pyridin-3-yl, 4-(CH₃NH—C(═O))-pyridin-3-yl, 4-((CH₃)₂N—C(═O))-pyridin-3-yl, 4-((-(1-methylazetidin-3-yl)-NH—C(═O)-)pyridin-3-yl, 4-((1-acetylazetidin-3-yl)-NH—C(═O)-)pyridin-3-yl, 4-((1-methylpyrrolidin-3-yl)-NH—C(═O)-)pyridin-3-yl (4-(1-methylpyrrolidin-3-ylcarbamoyl)pyridin-3-yl), 4-((1-methylpyrrolidin-3-yl)-N(CH₃)—C(═O)-)pyridin-3-yl, 4-(1-methyl-pyrrolidin-3-yl)-CH₂—NH—C(═O)-pyridin-3-yl (4-(1-methyl-pyrrolidin-3-ylmethylcarbamoyl)pyridin-3-yl), 4-(1-acetylpyrrolidin-3-yl)-NH—C(═O)-pyridin-3-yl, 4-(5-fluoro-1-methylpyrrolidin-3-yl)-NH—C(═O)-pyridin-3-yl, 4-(3-fluoro-1-methylpyrrolidin-3-yl)-NH—C(═O)-pyridin-3-yl, 4-(5,5-difluoro-1-methylpyrrolidin-3-yl)-NH—C(═O)-pyridin-3-yl, 4-(3,3-difluoro-1-methylpyrrolidin-3-yl)-NH—C(═O)-pyridin-3-yl, 4-(oxan-4-yl-NH—C(═O))pyridin-3-yl, 4-((1-methylpiperidin-4-yl)-NH—C(═O)-)pyridin-3-yl (4-(1-methylpiperidin-4-ylcarbamoyl)pyridin-3-yl), 4-((1-methylpiperidin-3-yl)-NH—C(═O)-)pyridin-3-yl (4-(1-methylpiperidin-3-ylcarbamoyl)pyridin-3-yl), 4-(((3S)-1-methyl-pyrrolidin-3-yl)-NH—C(═O)-)pyridin-3-yl, 4-(((3R)-1-methyl-pyrrolidin-3-yl)-NH—C(═O)-)pyridin-3-yl, 4-(1-acetylpiperidin-3-ylcarbamoyl)pyridin-3-yl, 4-(1-acetylpiperidin-4-ylcarbamoyl)pyridin-3-yl, 4-(1-acetylpiperidin-3-ylmethylcarbamoyl)pyridin-3-yl, 4-(1-acetylpiperidin-4-ylmethylcarbamoyl)pyridin-3-yl, 4-((1-acetylazetidin-3-yl)-CH₂—NH—C(═O)-)pyridin-3-yl (4-(1-acetylazetidin-3-ylmethylcarbamoyl)pyridin-3-yl), 4-(5-oxopyrrolidin-3-yl)-NH—C(═O)-pyridin-3-yl, 4-(2-oxopyrrolidin-3-yl)-NH—C(═O)-pyridin-3-yl, 4-(1-methyl-5-oxopyrrolidin-3-yl)-NH—C(═O)-pyridin-3-yl, 4-(1-methyl-2-oxopyrrolidin-3-yl)-NH—C(═O)-pyridin-3-yl, 4-(morpholin-3-yl)-CH₂—NH—C(═O)-pyridin-3-yl, 4-(4-methylmorpholin-2-yl)-CH₂—NH—CO-pyridin-3-yl, (4-acetylmorpholin-3-yl)-CH₂—NH—C(═O)-pyridin-3-yl, 4-acetylmorpholin-2-yl-CH₂—NH—C(═O)-pyridin-3-yl (4-acetylmorpholin-2-ylmethylcarbamoylpyridin-3-yl), 4-((2-oxopiperidin-4-yl)-NH—C(═O)-)pyridin-3-yl (4-(2-oxopiperidin-4-ylcarbamoyl)pyridin-3-yl), 4-((1-methyl-2-oxopiperidin-4-yl)-NH—C(═O)-)pyridin-3-yl (4-(1-methyl-2-oxopiperidin-4-ylcarbamoyl)pyridin-3-yl), 4-(1-methyl-6-oxopiperidin-3-yl)-NH—C(═O)-) pyridin-3-yl (4-(1-methyl-6-oxopiperidin-3-ylcarbamoyl)pyridin-3-yl, 4-(phenyl-NH—C(═O)-pyridin-3-yl (4-(phenylcarbamoyl)pyridin-3-yl), 4-((1-methyl-1H-pyrazol-4-yl)NH—C(═O))pyridin-3-yl, 4-((1-methylpyrazol-4-yl)-CH₂NH—C(═O))-pyridin-3-yl, 4-(pyridin-3-yl)-NH—C(═O)-pyridin-4-yl, 4-((1-methyl-imidazol-5-yl)-CH₂—NH—C(═O)-)pyridin-3-yl) (4-(1-methyl-imidazol-5-ylmethyl)carbamoylpyridin-3-yl), 4-((pyrimidin-4-yl)-NH—C(═O))pyridin-3-yl, 4-((pyrimidinyl-5-yl)-NHC(═O))-pyridin-3-yl, 4-((pyrimidinyl-5-yl)-CH₂NHC(═O))-pyridin-3-yl, 4-(pyridazin-3-ylmethylcarbamoyl)pyridin-3-yl, 4-methanesulfonyl-pyridin-1-ium-1-olate-3-yl, 2H,3H,4H-pyrido[4,3-b][1,4]oxazin-8-yl, 4-carbamoylpyrimidin-5-yl, 1-methyl-1H-1,2,3-triazol-5-yl, or 4-[(pyrimidin-5-yl)amino]methylpyridin-3-yl.

10. The compound according to claim 9, or derivatives, N-oxides, prodrugs, solvates, tautomers or stereoisomers thereof as well as the physiologically acceptable salts of each of the foregoing, including mixtures thereof in all ratios, wherein R¹ denotes 4-ethylphenyl, 2,5-dimethylphenyl, 3-methoxyphenyl, 4-fluorophenyl, 3-bromophenyl, 4-bromophenyl, 2-chloro-5-methoxy-phenyl, 3-amino-4-methylphenyl, 4-amino-3-fluorophenyl, dihydrobenzofuran-5-yl, N-methyl-indol-6-yl, 1-ethyl-1H-indol-6-yl, 2-(difluoromethyl)-1H-indol-6-yl, 1,4-dimethyl-1H-indol-6-yl, 1,5-dimethyl-1H-indol-6-yl, 4-fluoro-1-methylindol-6-yl, 5-fluoro-1-methylindol-6-yl, 7-fluoro-1-methyl-indol-6-yl, benzothiazol-6-yl, benzothiazol-5-yl, 3-methyl-1-benzofuran-5-yl, 3-methyl-1-benzothiophen-5-yl, 2,3-dihydrobenzo[1,4]dioxin-6-yl, 1-methyl-1H-pyrrolo[2,3-b]pyrdin-6-yl, 2-amino-1,3-benzothiazol-5-yl, 2-amino-1,3-benzothiazol-6-yl, 2-(pyrrolidin-2-yl-C(═O)—NH—)-1,3-benzothiazol-6-yl, or 2,1,3-benzothiadiazol-5-yl.

11. The compound according to claim 1, or derivatives, N-oxides and/or physiologically acceptable salts thereof, selected from the group consisting of:
8-(2,3-dihydro-1,4-benzodioxin-6-yl)-N-(4-methanesulfonylpyridin-3-yl)quinoxalin-6-amine,
5-(1-methyl-1H-indol-6-yl)-7-{1H,2H,3H-pyrrolo[2,3-c]pyridin-1-yl}quinoxaline,
N-(2-methanesulfonylphenyl)-8-(1-methyl-1H-indol-6-yl)quinoxalin-6-amine,
8-(1,3-benzothiazol-6-yl)-N-(4-methanesulfonylpyridin-3-yl)quinoxalin-6-amine,
8-(2-chloro-5-methoxyphenyl)-N-(4-methanesulfonylpyridin-3-yl)quinoxalin-6-amine,
N-(2-methanesulfonylpyridin-3-yl)-8-(1-methyl-1H-indol-6-yl)quinoxalin-6-amine,
8-(1-methyl-1H-indol-6-yl)-N-[2-(morpholine-4-sulfonyl)phenyl]quinoxalin-6-amine,
2-{[8-(1-methyl-1H-indol-6-yl)quinoxalin-6-yl]amino}benzene-1-sulfonamide,
8-(1,3-benzothiazol-5-yl)-N-(4-methanesulfonylpyridin-3-yl)quinoxalin-6-amine trifluoroacetate,
N-(5-bromo-2-methanesulfonylphenyl)-8-(1-methyl-1H-indol-6-yl)quinoxalin-6-amine,
N-(4-methanesulfonylpyridin-3-yl)-8-(1-methyl-1H-indol-6-yl)quinoxalin-6-amine,
N-(2-methoxypyridin-3-yl)-8-(1-methyl-1H-indol-6-yl)quinoxalin-6-amine,
3-{[8-(1-methyl-1H-indol-6-yl)quinoxalin-6-yl]amino}pyridin-2-ol,
8-(1-methyl-1H-indol-6-yl)-N-[2-(piperazine-1-sulfonyl)phenyl]quinoxalin-6-amine,
N-methyl-2-{[8-(1-methyl-1H-indol-6-yl)quinoxalin-6-yl]amino}benzene-1-sulfonamide,
3-N-[8-(1-methyl-1H-indol-6-yl)quinoxalin-6-yl]pyridine-2,3-diamine,
3-{[8-(1-methyl-1H-indol-6-yl)quinoxalin-6-yl]amino}pyridine-4-carbonitrile,
3-{[8-(1-methyl-1H-indol-6-yl)quinoxalin-6-yl]amino}pyridine-4-carboxamide,
N,N-dimethyl-2-{[8-(1-methyl-1H-indol-6-yl)quinoxalin-6-yl]amino}benzene-1-sulfonamide,
N-(2-methanesulfonylphenyl)-8-{1-methyl-1H-pyrrolo[2,3-b]pyridin-6-yl}quinoxalin-6-amine trifluoroacetate,
N-(4-methanesulfonylpyridin-3-yl)-8-(3-methyl-1-benzofuran-5-yl)quinoxalin-6-amine,
N-(4-methoxypyridin-3-yl)-8-(1-methyl-1H-indol-6-yl)quinoxalin-6-amine,
3-{[8-(3-methyl-1-benzofuran-5-yl)quinoxalin-6-yl]amino}pyridine-4-carbonitrile,
4-methanesulfonyl-3-{[8-(1-methyl-1H-indol-6-yl)quinoxalin-6-yl]amino}benzonitrile,
3-{[8-(3-methyl-1-benzofuran-5-yl)quinoxalin-6-yl]amino}pyridine-4-carboxamide,
N-(5-methanesulfonylpyrimidin-4-yl)-8-(1-methyl-1H-indol-6-yl)quinoxalin-6-amine,
3-{[8-(1-methyl-1H-indol-5-yl)quinoxalin-6-yl]amino}pyridine-4-carbonitrile,
3-{[8-(1-methyl-1H-indol-5-yl)quinoxalin-6-yl]amino}pyridine-4-carboxamide,
N-(4-chloropyridin-3-yl)-8-(1-methyl-1H-indol-5-yl)quinoxalin-6-amine,
8-(1-methyl-1H-indol-5-yl)-N-[4-(1-methyl-1H-pyrazol-4-yl)pyridin-3-yl]quinoxalin-6-amine,
8-(1-methyl-1H-indol-5-yl)-N-[4-(4-methylpiperazin-1-yl)pyridin-3-yl]quinoxalin-6-amine,
8-(1-methyl-1H-indol-5-yl)-N-[4-(pyrimidin-5-yl)pyridin-3-yl]quinoxalin-6-amine,
5-(1-methyl-1H-indol-5-yl)-7-{1H,2H,3H-pyrrolo[2,3-c]pyridin-1-yl}quinoxaline,
N-(2-methanesulfonyl-5-nitrophenyl)-8-(1-methylindol-6-yl)quinoxalin-6-amine,
6-methanesulfonyl-N1-[8-(1-methyl-1H-indol-6-yl)quinoxalin-6-yl]benzene-1,3-diamine,
8-(2,3-dihydro-1-benzofuran-5-yl)-N-(4-methanesulfonylpyridin-3-yl)quinoxalin-6-amine,
N-[5-(aminomethyl)-2-methanesulfonylphenyl]-8-(1-methyl-1H-indol-5-yl)quinoxalin-6-amine,
8-(2,5-dimethylphenyl)-N-(4-methanesulfonylpyridin-3-yl)quinoxalin-6-amine,
8-(1-methyl-1H-indol-6-yl)-N-[4-(4-methylpiperazin-1-yl)pyridin-3-yl]quinoxalin-6-amine,
N-(4-methanesulfonyl-3-{[8-(1-methyl-1H-indol-6-yl) quinoxalin-6-yl]amino}phenyl)acetamide,
N-[5-(1H-imidazol-1-yl)-2-methanesulfonylphenyl]-8-(1-methyl-1H-indol-6-yl)quinoxalin-6-amine,
N-[2-methanesulfonyl-5-(2H-1,2,3,4-tetrazol-5-yl)phenyl]-8-(1-methyl-1H-indol-6-yl)quinoxalin-6-amine,
4-methanesulfonyl-3-{[8-(1-methyl-1H-indol-6-yl)quinoxalin-6-yl]amino}pyridin-1-ium-1-olate,
N-[2-methanesulfonyl-5-(4-methylpiperazin-1-yl)phenyl]-8-(1-methyl-1H-indol-6-yl)quinoxalin-6-amine,
1-[4-(4-methanesulfonyl-3-{[8-(1-methyl-1H-indol-6-yl)quinoxalin-6-yl]amino}phenyl)piperazin-1-yl]ethan-1-one,
3-{[8-(1-methyl-1H-indol-6-yl)quinoxalin-6-yl]oxy}pyridine-4-carbonitrile,
N-(4-methanesulfonylpyridin-3-yl)-8-[3-(1H-1,2,3-triazol-4-yl)phenyl]quinoxalin-6-amine,
N-(4-methanesulfonylpyridin-3-yl)-8-[1-(propan-2-yl)-1H-indol-6-yl]quinoxalin-6-amine,
8-[3-(dimethylamino)phenyl]-N-(4-methanesulfonylpyridin-3-yl)quinoxalin-6-amine,
N-(4-methanesulfonylpyridin-3-yl)-8-(3-methylphenyl)quinoxalin-6-amine,
N-methyl-3-{[8-(1-methyl-1H-indol-6-yl)quinoxalin-6-yl]amino}pyridine-4-carboxamide,
N,N-dimethyl-3-{[8-(1-methyl-1H-indol-6-yl)quinoxalin-6-yl]amino}pyridine-4-carboxamide,
3-{[8-(1-methyl-1H-indol-6-yl)quinoxalin-6-yl]amino}-N-(pyrimidin-5-yl)pyridine-4-carboxamide,
3-{[8-(1-methyl-1H-indol-6-yl)quinoxalin-6-yl]amino}-N-(pyrimidin-5-ylmethyl)pyridine-4-carboxamide,
3-{[8-(1-methyl-1H-indol-6-yl)quinoxalin-6-yl]amino}-N-[(1-methyl-1H-pyrazol-4-yl)methyl]pyridine-4-carboxamide,
4-methanesulfonyl-N1-methyl-N3-[8-(1-methyl-1H-indol-6-yl)quinoxalin-6-yl]benzene-1,3-diamine, 8-[3-(chloromethyl)-1-benzofuran-5-yl]-N-(4-methanesulfonylpyridin-3-yl)quinoxalin-6-amine,
8-(7-fluoro-1-methyl-1H-indol-6-yl)-N-(4-methanesulfonylpyridin-3-yl)quinoxalin-6-amine,
8-(4-ethylphenyl)-N-(4-methanesulfonylpyridin-3-yl)quinoxalin-6-amine,
8-(1H-1,3-benzodiazol-5-yl)-N-(4-methanesulfonylpyridin-3-yl)quinoxalin-6-amine,
N-(4-methanesulfonylpyridin-3-yl)-8-(3-methoxyphenyl)quinoxalin-6-amine,
8-(3,3-dimethyl-2,3-dihydro-1-benzofuran-5-yl)-N-(4-methanesulfonylpyridin-3-yl)quinoxalin-6-amine,
8-(3-ethylphenyl)-N-(4-methanesulfonylpyridin-3-yl)quinoxalin-6-amine,
8-(2-amino-5-methylphenyl)-N-(4-methanesulfonylpyridin-3-yl)quinoxalin-6-amine,
2-{7-[(4-methanesulfonylpyridin-3-yl)amino]quinoxalin-5-yl}-4-methylphenol,
8-(1-methyl-1H-indol-6-yl)-N-[4-(1H-1,2,3,4-tetrazol-5-yl)pyridin-3-yl]quinoxalin-6-N-(4-chloropyridin-3-yl)-8-(1-methyl-1H-indol-6-yl)quinoxalin-6-amine,
8-(4-fluoro-1-methyl-1H-indol-6-yl)-N-(4-methanesulfonylpyridin-3-yl)quinoxalin-6-amine,
4-methanesulfonyl-3-{[8-(3-methyl-1-benzofuran-5-yl)quinoxalin-6-yl]amino}pyridin-1-ium-1-olate,
8-(5-fluoro-1-methyl-1H-indol-6-yl)-N-(4-methanesulfonylpyridin-3-yl)quinoxalin-6-amine,
N-(4-methanesulfonylpyridin-3-yl)-8-(2-methoxy-5-methylphenyl)quinoxalin-6-amine,
8-(3-amino-4-methylphenyl)-N-(4-methanesulfonylpyridin-3-yl)quinoxalin-6-amine,
N-(3-methanesulfonylpyridin-2-yl)-8-(1-methyl-1H-indol-6-yl)quinoxalin-6-amine,
1-[4-(3-{[8-(1-methyl-1H-indol-6-yl)quinoxalin-6-yl]amino}pyridin-4-yl)piperazin-1-yl]ethan-1-one,
N-[4-(1-methyl-1H-imidazol-4-yl)pyridin-3-yl]-8-(1-methyl-1H-indol-6-yl)quinoxalin-6-amine,
8-(1-methyl-1H-indol-6-yl)-N-{2H,3H,4H-pyrido[4,3-b][1,4]oxazin-8-yl}quinoxalin-6-amine,
2-{[8-(1-methyl-1H-indol-6-yl)quinoxalin-6-yl]amino}-N-[(pyrimidin-5-yl)methyl]benzene-1-sulfonamide,
2-{[8-(1-methyl-1H-indol-6-yl)quinoxalin-6-yl]amino}benzonitrile,
2-{[8-(1-methyl-1H-indol-6-yl)quinoxalin-6-yl]amino}benzamide, 4-cyano-3-{[8-(1-methyl-1H-indol-6-yl)quinoxalin-6-yl]amino}pyridin-1-ium-1-olate,
3-{methyl[8-(1-methyl-1H-indol-6-yl)quinoxalin-6-yl]amino}pyridine-4-carbonitrile,
3-{[8-(1-methyl-1H-indol-6-yl)quinoxalin-6-yl]amino}-N-(1-methyl-1H-pyrazol-4-yl)pyridine-4-carboxamide,
N-[2-methanesulfonyl-5-(1-methyl-1H-pyrazol-5-yl)phenyl]-8-(1-methyl-1H-indol-6-yl)quinoxalin-6-amine,
N-[2-methanesulfonyl-5-(1,3-oxazol-2-yl)phenyl]-8-(1-methyl-1H-indol-6-yl)quinoxalin-6-amine,
3-{methyl[8-(1-methyl-1H-indol-6-yl)quinoxalin-6-yl]amino}pyridine-4-carboxamide,
3-{[8-(1-methyl-1H-indol-6-yl)quinoxalin-6-yl]amino}-N-phenylpyridine-4-carboxamide,
3-{[8-(1-methyl-1H-indol-6-yl)quinoxalin-6-yl]amino}-N-(1-methyl-2-oxopiperidin-4-yl)pyridine-4-carboxamide,
N-(1-acetylazetidin-3-yl)-3-{[8-(1-methyl-1H-indol-6-yl)quinoxalin-6-yl]amino}pyridine-4-carboxamide,
3-{[8-(1-methyl-1H-indol-6-yl)quinoxalin-6-yl]amino}-N-(1-methylpyrrolidin-3-yl)pyridine-4-carboxamide,
2-{[8-(1-methyl-1H-indol-6-yl)quinoxalin-6-yl]amino}-N-(pyrimidin-5-yl)benzene-1-sulfonamide,
3-{[8-(1-methyl-1H-indol-6-yl)quinoxalin-6-yl]amino}-N-(oxan-4-yl)pyridine-4-carboxamide,
6-methanesulfonyl-N1-[8-(3-methyl-1-benzofuran-5-yl)quinoxalin-6-yl]benzene-1,3-diamine,
N-(2-methanesulfonyl-5-nitrophenyl)-8-(3-methyl-1-benzofuran-5-yl)quinoxalin-6-amine,
N-(4-methanesulfonylpyridin-3-yl)-N-methyl-8-(1-methyl-1H-indol-6-yl)quinoxalin-6-amine,
N-(4-methanesulfonylpyridin-3-yl)-8-(3-methyl-1-benzothiophen-5-yl)quinoxalin-6-amine,
Methyl 4-methanesulfonyl-3-{[8-(1-methyl-1H-indol-6-yl)quinoxalin-6-yl]amino}benzoate,
4-Methanesulfonyl-3-{[8-(1-methyl-1H-indol-6-yl)quinoxalin-6-yl]amino}benzamide,
8-(2,1,3-benzothiadiazol-5-yl)-N-(4-methanesulfonylpyridin-3-yl)quinoxalin-6-amine,
8-(1H-1,2,3-benzotriazol-5-yl)-N-(4-methanesulfonylpyridin-3-yl)quinoxalin-6-amine,
4-methanesulfonyl-3-{[8-(1-methyl-1H-indol-6-yl)quinoxalin-6-yl]amino}benzohydrazide,
8-(2,1,3-benzoxadiazol-5-yl)-N-(4-methanesulfonylpyridin-3-yl)quinoxalin-6-amine,
N-(1-acetylpyrrolidin-3-yl)-3-{[8-(1-methyl-1H-indol-6-yl)quinoxalin-6-yl]amino}pyridine-4-carboxamide,
3-{[8-(1-methyl-1H-indol-6-yl)quinoxalin-6-yl]amino}-N-(1-methyl-6-oxopiperidin-3-yl)pyridine-4-carboxamide,
3-{[8-(1-methyl-1H-indol-6-yl)quinoxalin-6-yl]amino}-N-(1-methylpiperidin-4-yl)pyridine-4-carboxamide,
3-{[8-(1-methyl-1H-indol-6-yl)quinoxalin-6-yl]amino}-N-(1-methylpiperidin-3-yl)pyridine-4-carboxamide,
3-{methyl[8-(1-methyl-1H-indol-6-yl)quinoxalin-6-yl]amino}-N-(pyrimidin-5-yl)pyridine-4-carboxamide,
N-cyclohexyl-3-{[8-(1-methyl-1H-indol-6-yl)quinoxalin-6-yl]amino}pyridine-4-carboxamide,
3-{[8-(1-methyl-1H-indol-6-yl)quinoxalin-6-yl]amino}-N-(2-oxopiperidin-4-yl)pyridine-4-carboxamide,
2-{7-[(4-methanesulfonylpyridin-3-yl)amino]quinoxalin-5-yl}-4-methylbenzamide,
8-(3-ethoxyphenyl)-N-(4-methanesulfonylpyridin-3-yl)quinoxalin-6-amine,
N-(4-methanesulfonylpyridin-3-yl)-8-[3-(propan-2-yloxy)phenyl]quinoxalin-6-amine,
8-(4-aminophenyl)-N-(4-methanesulfonylpyridin-3-yl)quinoxalin-6-amine,
8-(3-aminophenyl)-N-(4-methanesulfonylpyridin-3-yl)quinoxalin-6-amine,
butyl 3-{[8-(1-methyl-1H-indol-6-yl)quinoxalin-6-yl]amino}pyridine-4-carboxylate,
3-{[8-(1-methyl-1H-indol-6-yl)quinoxalin-6-yl]amino}-N-[(morpholin-3-yl)methyl]pyridine-4-carboxamide,
N-[(4-acetylmorpholin-3-yl)methyl]-3-{[8-(1-methyl-1H-indol-6-yl)quinoxalin-6-yl]amino}pyridine-4-carboxamide,
3-{[8-(1-methyl-1H-indol-6-yl)quinoxalin-6-yl]amino}-N-[(4-methylmorpholin-2-yl)methyl]pyridine-4-carboxamide,
N-[(1-acetylazetidin-3-yl)methyl]-3-{[8-(1-methyl-1H-indol-6-yl)quinoxalin-6-yl]amino}pyridine-4-carboxamide,
N-[(4-acetylmorpholin-2-yl)methyl]-3-{[8-(1-methyl-1H-indol-6-yl)quinoxalin-6-yl]amino}pyridine-4-carboxamide,
3-{[8-(1-methyl-1H-indol-6-yl)quinoxalin-6-yl]amino}-N-[(1-methylpyrrolidin-3-yl)methyl]pyridine-4-carboxamide,
N-[(1-methyl-1H-imidazol-5-yl)methyl]-3-{[8-(1-methyl-1H-indol-6-yl)quinoxalin-6-yl]amino}pyridine-4-carboxamide,
3-{[8-(1-methyl-1H-indol-6-yl)quinoxalin-6-yl]amino}-N-[(pyridazin-3-yl)methyl]pyridine-4-carboxamide,
4-{[8-(1-methyl-1H-indol-6-yl)quinoxalin-6-yl]amino}pyridine-3-carbonitrile,
N-(1-acetylpiperidin-4-yl)-3-{[8-(1-methyl-1H-indol-6-yl)quinoxalin-6-yl]amino}pyridine-4-carboxamide,
N-(1-acetylpiperidin-3-yl)-3-{[8-(1-methyl-1H-indol-6-yl)quinoxalin-6-yl]amino}pyridine-4-carboxamide,
5-{[8-(1-methyl-1H-indol-6-yl)quinoxalin-6-yl]amino}pyrimidine-4-carboxamide,
3-{amino}pyridine-4-pyridine-4-carb carbonitrile,
3-{[8-(3-methyl-1-benzothiophen-5-yl)quinoxalin-6-yl]amino}-N-(1-methylpyrrolidin-3-yl)pyridine-4-carboxamide,
N-(4-methanesulfonylpyridin-3-yl)-8-(4-methoxyphenyl)quinoxalin-6-amine,
N-(4-methanesulfonylpyridin-3-yl)-8-(5-methoxy-2-methylphenyl)quinoxalin-6-amine,
8-[1-(difluoromethyl)-1H-indol-6-yl]-N-(4-methanesulfonylpyridin-3-yl)quinoxalin-6-amine,
8-(4-bromophenyl)-N-(4-methanesulfonylpyridin-3-yl)quinoxalin-6-amine,
8-(3-bromophenyl)-N-(4-methanesulfonylpyridin-3-yl)quinoxalin-6-amine,
2-aminopyrimidin-4-yl 3-{[8-(1-methyl-1H-indol-6-yl)quinoxalin-6-yl]amino}pyridine-4-carboxylate,
8-(1,2-benzothiazol-5-yl)-N-(4-methanesulfonylpyridin-3-yl)quinoxalin-6-amine,
8-(2-amino-1,3-benzothiazol-5-yl)-N-(4-methanesulfonylpyridin-3-yl)quinoxalin-6-N-(4-methanesulfonylpyridin-3-yl)-8-[3-(trifluoromethoxy)phenyl]quinoxalin-6-amine,
N-(4-{7-[(4-methanesulfonylpyridin-3-yl)amino]quinoxalin-5-yl}phenyl)pyrrolidine-2-carboxamide,
N-(3-{7-[(4-methanesulfonylpyridin-3-yl)amino]quinoxalin-5-yl}phenyl)pyrrolidine-2-carboxamide,
8-(1-ethyl-1H-indol-6-yl)-N-(4-methanesulfonylpyridin-3-yl)quinoxalin-6-amine,
N-(4-methanesulfonylpyridin-3-yl)-8-(1-methyl-1H-1,2,3-benzotriazol-5-yl)quinoxalin-6-amine,
2-{[8-(1-methyl-1H-indol-6-yl)quinoxalin-6-yl]amino}-N-[(1-methylpyrrolidin-3-yl)methyl]benzene-1-sulfonamide,
N-(4-methanesulfonylpyridin-3-yl)-8-(2-methyl-1,3-benzothiazol-5-yl)quinoxalin-6-amine,
N-(4-methanesulfonylpyridin-3-yl)-8-(1-methyl-1H-1,2,3-benzotriazol-6-yl)quinoxalin-6-amine,
2-{[8-(1-methyl-1H-indol-6-yl)quinoxalin-6-yl]amino}-N-(1-methylpyrrolidin-3-yl)benzene-1-sulfonamide,
2-{[8-(1-methyl-1H-indol-6-yl)quinoxalin-6-yl]amino}-N-[(oxan-4-yl)methyl]benzene-1-sulfonamide,
2-{[8-(1-methyl-1H-indol-6-yl)quinoxalin-6-yl]amino}-N-[(1-methyl-1H-pyrazol-4-yl)methyl]benzene-1-sulfonamide,
8-(2-amino-1,3-benzothiazol-6-yl)-N-(4-methanesulfonylpyridin-3-yl)quinoxalin-6-amine,
N-{4-[(dimethylamino)methyl]pyridin-3-yl}-8-(1-methyl-1H-indol-6-yl)quinoxalin-6-amine,
N-{2-[(dimethylamino)methyl]phenyl}-8-(1-methyl-1H-indol-6-yl)quinoxalin-6-amine,
2-{[8-(1-methyl-1H-indol-6-yl)quinoxalin-6-yl]amino}benzoic,
3-{[8-(1-methyl-1H-indol-6-yl)quinoxalin-6-yl]amino}pyridine-4-carboxylic acid,
3-{[8-(1-methyl-1H-indol-6-yl)quinoxalin-6-yl]amino}-N-(1-methylazetidin-3-yl)pyridine-4-carboxamide,
N-methyl-3-{[8-(1-methyl-1H-indol-6-yl)quinoxalin-6-yl]amino}-N-(1-methylpyrrolidin-3-yl)pyridine-4-carboxamide,
2-{[8-(1-methyl-1H-indol-6-yl)quinoxalin-6-yl]amino}-N-(1-methylpyrrolidin-3-yl)benzamide,
N-(5-{7-[(4-methanesulfonylpyridin-3-yl)amino]quinoxalin-5-yl}-1,3-benzothiazol-2-yl)pyrrolidine-2-carboxamide,
N-(4-methanesulfonylpyridin-3-yl)-8-(1-propyl-1H-indol-6-yl)quinoxalin-6-amine,
N-(6-{7-[(4-methanesulfonylpyridin-3-yl)amino]quinoxalin-5-yl}-1,3-benzothiazol-2-yl)pyrrolidine-2-carboxamide,
N-(4-methanesulfonylpyridin-3-yl)-8-[4-(trifluoromethyl)phenyl]quinoxalin-6-amine,
8-(4-amino-3-fluorophenyl)-N-(4-methanesulfonylpyridin-3-yl)quinoxalin-6-amine,
N-methyl-2-{[8-(1-methyl-1H-indol-6-yl)quinoxalin-6-yl]amino}-N-[(pyrimidin-5-yl)methyl]benzene-1-sulfonamide,
8-(4-fluorophenyl)-N-(4-methanesulfonylpyridin-3-yl)quinoxalin-6-amine,
8-(1,4-dimethyl-1H-indol-6-yl)-N-(4-methanesulfonylpyridin-3-yl)quinoxalin-6-amine,
8-(2-amino-1,3-benzothiazol-5-yl)-N-(2-methanesulfonylphenyl)quinoxalin-6-amine,
N-(2-methanesulfonylphenyl)-8-(3-methyl-1-benzothiophen-5-yl)quinoxalin-6-amine,
8-(3,5-diethylphenyl)-N-(4-methanesulfonylpyridin-3-yl)quinoxalin-6-amine,
3-{[8-(3-methyl-1-benzothiophen-5-yl)quinoxalin-6-yl]amino}-N-[(3 S)-1-methylpyrrolidin-3-yl]pyridine-4-carboxamide,
3-{[8-(3-methyl-1-benzothiophen-5-yl)quinoxalin-6-yl]amino}-N-[(3R)-1-methylpyrrolidin-3-yl]pyridine-4-carboxamide,
8-[2-(dimethylamino)-5-methylphenyl]-N-(4-methanesulfonylpyridin-3-yl)quinoxalin-6-amine,
N-(1-methyl-1H-1,2,3-triazol-5-yl)-8-(1-methyl-1H-indol-6-yl)quinoxalin-6-amine,
8-(1,5-dimethyl-1H-indol-6-yl)-N-(4-methanesulfonylpyridin-3-yl)quinoxalin-6-amine,
3-{[8-(4-fluoro-1-methyl-1H-indol-6-yl)quinoxalin-6-yl]amino}-N-(1-methylpyrrolidin-3-yl)pyridine-4-carboxamide,
3-{[8-(1-methyl-1H-indol-6-yl)quinoxalin-6-yl]oxy}pyridine-4-carboxylic acid,
2-{[8-(3-methyl-1-benzothiophen-5-yl)quinoxalin-6-yl]amino}-N-(1-methylpyrrolidin-3-yl)benzene-1-sulfonamide,
N-(5-{7-[(4-methanesulfonylpyridin-3-yl)amino]quinoxalin-5-yl}-1-benzothiophen-2-yl)acetamide,
8-[2-(dimethylamino)-1,3-benzothiazol-5-yl]-N-(4-methanesulfonylpyridin-3-yl)quinoxalin-6-amine,
3-{[8-(1-methyl-1H-indol-6-yl)quinoxalin-6-yl]amino}-N-(pyrimidin-4-yl)pyridine-4-carboxamide,
N-(1-acetylazetidin-3-yl)-3-{[8-(3-methyl-1-benzothiophen-5-yl)quinoxalin-6-yl]amino}pyridine-4-carboxamide,
8-(1-methyl-1H-indol-6-yl)-N-(4-{[(pyrimidin-5-yl)amino]methyl}pyridin-3-yl)quinoxalin-6-amine,
2-{[8-(1-methyl-1H-indol-6-yl)quinoxalin-6-yl]amino}-N-(1-methylpiperidin-3-yl)-benzene-1-sulfonamide,
2-{[8-(1-methyl-1H-indol-6-yl)quinoxalin-6-yl]amino}-N-(oxan-3-yl)benzene-1-sulfonamide,
N-(4-methanesulfonylpyridin-3-yl)-8-[3-(methyl sulfanyl)phenyl]quinoxalin-6-amine,
N-(4-methanesulfonylpyridin-3-yl)-8-[3-(trifluoromethyl)-1-benzothiophen-5-yl]-quinoxalin-6-amine,
8-(4-bromo-3-fluorophenyl)-N-(4-methanesulfonylpyridin-3-yl)quinoxalin-6-amine,
8-(4-bromo-2-methylphenyl)-N-(4-methanesulfonylpyridin-3-yl)quinoxalin-6-amine,
N-(4-methanesulfonylpyridin-3-yl)-8-[4-(pentafluoro-λ⁶-sulfanyl)phenyl]quinoxalin-6-amine,
3-{[8-(2-amino-1,3-benzothiazol-5-yl)quinoxalin-6-yl]amino}-N-(1-methylpyrrolidin-3-yl)pyridine-4-carboxamide,
3-{[8-(4-bromophenyl)quinoxalin-6-yl]amino 1-N-(1-methylpyrrolidin-3-yl)pyridine-4-carboxamide,
N-(4-methanesulfonylpyridin-3-yl)-8-[2-(methyl amino)-1,3-benzothiazol-5-yl]quinoxalin-6-amine,
5-{7-[(4-methanesulfonylpyridin-3-yl)amino]quinoxalin-5-yl}-2,3-dihydro-1,3-benzothiazol-2-one (5-{7-[(4-methanesulfonylpyridin-3-yl)amino]quinoxalin-5-yl}-1,3-benzothiazol-2-ol),
8-(2-amino-1-benzothiophen-5-yl)-N-(4-methanesulfonylpyridin-3-yl)quinoxalin-6-amine,
8-(1-methyl-1H-indol-6-yl)-N-{4-[(methylamino)methyl]pyridin-3-yl}quinoxalin-6-amine,
8-(3-methyl-1-benzothiophen-5-yl)-N-{4-[(methylamino)methyl]pyridin-3-yl}quinoxalin-6-amine,
N-(5-bromopyrimidin-4-yl)-8-(1-methyl-1H-indol-6-yl)quinoxalin-6-amine,
3-{[8-(3-methyl-1-benzothiophen-5-yl)quinoxalin-6-yl]amino}pyridine-4-carboxamide,
3-{[8-(1-methyl-1H-indol-6-yl)quinoxalin-6-yl]amino}-N-(pyridin-3-yl)pyridine-4-carboxamide,
8-(2-amino-1-benzothiophen-6-yl)-N-(4-methanesulfonylpyridin-3-yl)quinoxalin-6-amine,
3-{[8-(1-methyl-1H-indol-6-yl)quinoxalin-6-yl]oxy}pyridine-4-carboxamide, 3-{[8-(3-methyl-1-benzothiophen-5-yl)quinoxalin-6-yl]amino}-N-(5-oxopyrrolidin-3-yl)pyridine-4-carboxamide,
3-{[8-(3-methyl-1-benzothiophen-5-yl)quinoxalin-6-yl]amino}-N-(2-oxopyrrolidin-3-yl)pyridine-4-carboxamide,
3-{[8-(3-methyl-1-benzothiophen-5-yl)quinoxalin-6-yl]amino}-N-(1-methyl-5-oxopyrrolidin-3-yl)pyri dine-4-carboxamide,
3-{[8-(3-methyl-1-benzothiophen-5-yl)quinoxalin-6-yl]amino}-N-(1-methyl-2-oxopyrrolidin-3-yl)pyri dine-4-carboxamide,
8-(1-methyl-1H-indol-6-yl)-N-{4-[(methylamino)methyl]pyridin-3-yl}quinoxalin-6-amine,
N-methyl-3-{[8-(3-methyl-1-benzothiophen-5-yl)quinoxalin-6-yl]amino}pyridine-4-carboxamide,
3-{[8-(4-bromophenyl)quinoxalin-6-yl]amino}-N-(1-methylpyrrolidin-3-yl)pyridine-4-carboxamide,
3-{[8-(2-amino-1,3-benzothiazol-5-yl)quinoxalin-6-yl]amino}-N-(1-methyl-pyrrolidin-3-yl)pyridine-4-carboxamide,
N-(4-methanesulfonylpyridin-3-yl)-8-[4-(pentafluoro-λ⁶-sulfanyl)phenyl]quinoxalin-6-amine,
3-{[8-(1-methyl-1H-indol-6-yl)quinoxalin-6-yl]amino}-N-(pyrimidin-4-yl)pyridine-4-carboxamide,
8-(1-methyl-1H-indol-6-yl)-N-(4-{[(pyrimidin-5-yl)amino]methyl}pyridin-3-yl)quinoxalin-6-amine,
2-{[8-(1-methyl-1H-indol-6-yl)quinoxalin-6-yl]amino}-N-(1-methylpiperidin-3-yl)benzene-1-sulfonamide,
2-{[8-(1-methyl-1H-indol-6-yl)quinoxalin-6-yl]amino}-N-(oxan-3-yl)benzene-1-sulfonamide,
N-(4-methanesulfonylpyridin-3-yl)-8-[3-(methylsulfanyl)phenyl]quinoxalin-6-amine,
8-(4-bromo-3-fluorophenyl)-N-(4-methanesulfonylpyridin-3-yl)quinoxalin-6-amine,
8-(4-bromo-2-methylphenyl)-N-(4-methanesulfonylpyridin-3-yl)quinoxalin-6-amine,
2-amino-N-(5-{7-[(4-methanesulfonylpyridin-3-yl)amino]quinoxalin-5-yl}-1-benzothiophen-2-yl)acetamide,
N-(5-fluoro-1-methylpyrrolidin-3-yl)-3-{[8-(3-methyl-1-benzothiophen-5-yl)quinoxalin-6-yl]amino}pyridine-4-carboxamide,
N-(3-fluoro-1-methylpyrrolidin-3-yl)-3-{[8-(3-methyl-1-benzothiophen-5-yl)quinoxalin-6-yl]amino}pyridine-4-carboxamide,
N-(5,5-difluoro-1-methylpyrrolidin-3-yl)-3-{[8-(3-methyl-1-benzothiophen-5-yl)quinoxalin-6-yl]amino}pyridine-4-carboxamide and,
N-(3,3-difluoro-1-methylpyrrolidin-3-yl)-3-{[8-(3-methyl-1-benzothiophen-5-yl)quinoxalin-6-yl]amino}pyridine-4-carboxamide.

12. A pharmaceutical composition comprising at least one compound of formula (I) as defined in claim 1, or its derivatives, N-oxides, prodrugs, solvates, tautomers or stereoisomers thereof as well as the physiologically acceptable salts of each of the foregoing, including mixtures thereof in all ratios, as active ingredient, together with a pharmaceutically acceptable carrier.

13. The pharmaceutical composition according to claim 12 that further comprises a second active ingredient or its derivatives, N-oxides, prodrugs, solvates, tautomers or stereoisomers thereof as well as the physiologically acceptable salts of each of the foregoing, including mixtures thereof in all ratios, wherein that second active ingredient is other than the compound of formula (I) as defined in claim 1.

14. A medicament comprising at least one compound of formula (I) as defined in claim 1, or its derivatives, N-oxides, prodrugs, solvates, tautomers or stereoisomers thereof as well as the physiologically acceptable salts of each of the foregoing, including mixtures thereof in all ratios.

15. A method for preventing and/or treating medical conditions that are affected by inhibiting 6-phosphofructo-2-kinase/fructose-2,6-bisphosphatase (PFKFB) comprising administering to a patient in need thereof the compound of formula (I) as defined in claim 1, or its derivatives, N-oxides, prodrugs, solvates, tautomers or stereoisomers thereof as well as the physiologically acceptable salts of each of the foregoing, including mixtures thereof.

16. A method for preventing and/or treating cancer comprising administering to a patient in need thereof the compound of formula (I) as defined in claim 1, or its derivatives, N-oxides, prodrugs, solvates, tautomers or stereoisomers thereof as well as the physiologically acceptable salts of each of the foregoing, including mixtures thereof.

17. A kit comprising separate packs of a) an effective amount of a compound of formula (I) as defined in claim 1, or its derivatives, N-oxides, prodrugs, solvates, tautomers or stereoisomers thereof as well as the physiologically acceptable salts of each of the foregoing, including mixtures thereof in all ratios; and b) an effective amount of a further active ingredient that further active ingredient not being a compound of formula (I) as defined in claim 1.

18. Process A process for manufacturing a compound according to claim 1, or derivatives, N-oxides, prodrugs, solvates, tautomers or stereoisomers thereof as well as the physiologically acceptable salts of each of the foregoing, the process being characterized in that
(a) a compound of formula (II) 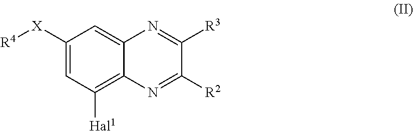
wherein HaI¹ denotes Cl, Br or I; and R², R³, R⁴, and X have the same meaning as
defined in claim 1 for compounds of formula (I);
is reacted under C—C coupling reaction conditions which conditions may utilize one or more suitable C—C coupling reaction reagents including catalysts with
a compound R¹-RG^{a} wherein R¹ has the same meaning as defined in claim 1 for compounds of formula (I); and RG^{a} denotes a chemical moiety being reactive under the particular C—C coupling reaction conditions utilized;
or
(b) a compound of formula (III) 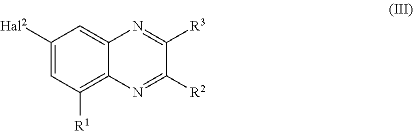
wherein HaI² denotes Cl, Br or I; and R¹, R², and R³ have the same meaning as defined in claim 1 for compounds of formula (I);
is reacted under C—N coupling reaction conditions which conditions may utilize one or more suitable C—N coupling reaction reagents including catalysts with
a compound R⁴—NHR⁵, wherein; R⁴ and R⁵ have the same meaning as defined in claim 1 for compounds of formula (I);
or
(c) a compound of formula (III) 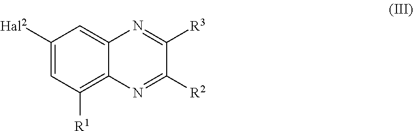
wherein HaI² denotes Cl, Br or I; and R¹, R², and R³ have the same meaning as defined in claim 1 for compounds of formula (I);
is reacted under C—O coupling reaction conditions which conditions may utilize one or more suitable C—O coupling reaction reagents including catalysts with
a compound R⁴—OH, wherein R⁴ has the same meaning as defined in claim 1 for compounds of formula (I).

19. A compound of formula (II) or (III) 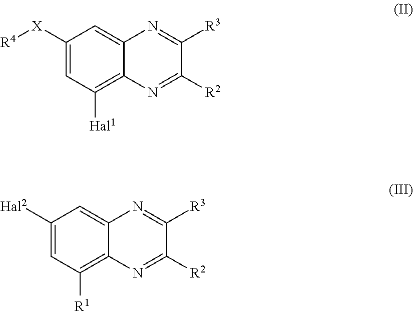
or salts thereof,
wherein
HaI¹ and HaI² denote independently from each other Cl, Br or I; and,
R¹, R², R³, R⁴, and X have the same meaning as defined in claim 1 for compounds of formula (I).

20. The method according to claim 16, wherein said cancer is selected from the group consisting of adipose cancer, anogenital cancer, astrocytoma cancer, bladder cancer, breast cancer, central nervous system cancer, cervical cancer, colon cancer, connective tissue cancer, glioblastoma, glioma, kidney cancer, leukemia, lung cancer, lymphoid cancer, ovarian cancer, pancreatic cancer, prostate cancer, retinal cancer, skin cancer, stomach cancer, thyroid cancer, and uterine cancer.
